SEQUENCE LISTING (1 sequence)

SEQ ID NO: 1 - protein, 353 aa, synthetic construct
GGPVRVGFYDIEGTLGKGNFAVVKLGRHRITKTEVAIKIIDKSQLDAVNLEKIYREVQIMKMLDHPHIIKLYQVMETKSMLYLVTEYAKNGEIFDYLANHGRLNESEARRKFWQILSAVDYCHGRKIVHRDLKAENLLLDNNMNIKIADFGFGNFFKSGELLATWCGSPPYAAPEVFEGQQYEGPQLDIWSMGVVLYVLVCGALPFDGPTLPILRQRVLEGRFRIPYFMSEDCEHLIRRMLVLDPSKRLTIAQIKEHKWMLIEVPVQRPVLYPQEQENEPSIGEFNEQVLRLMHSLGIDQQKTIESLQNKSYNHFAAIYFLLVERLKSHRSSFPVEQRLLVPRGSDYKDDDDK

(19) United States
(12) Patent Application Publication
(10) Pub. No.: US 2026/0274820 A1
(43) Pub. Date: Sep. 17, 2026

(54) SIK2 MODULATORS AND USES THEREOF

(71) Applicant: NIMBUS SALACIA, INC., Boston, MA (US)

(72) Inventors: Derun Li, Boston, MA (US); Leela Dodda, Boston, MA (US); Scott D. Edmondson, Boston, MA (US); Nathan E. Genung, Boston, MA (US); Alan Graves, Boston, MA (US); Neelu Kaila, Boston, MA (US); Daniel Joseph Price, Boston, MA (US); Ivan V. Sergeyev, Boston, MA (US); Md Munan Shaik, Boston, MA (US); Sekhar Surapaneni, Somerset, NJ (US); Sebastien Campos, Hoddesdon (GB); Russell George Dushin, Mystic, CT (US); Bruce Lefker, Mystic, CT (US); Steffen Gressies, Berlin (DE); Nikolay Sitnikov, Berlin (DE)

(21) Appl. No.: 19/559,446

(22) Filed: Mar. 6, 2026

Related U.S. Application Data
(60) Provisional application No. 63/768,684, filed on Mar. 7, 2025.
(60) Provisional application No. 63/848,808, filed on Jul. 22, 2025.
(60) Provisional application No. 63/979,850, filed on Feb. 10, 2026.

(51) Int. Cl. C07D 401/14; A61K 31/444; A61K 31/4545; A61K 31/4709; A61K 31/473; A61K 31/497; A61K 31/4985; A61K 31/501; A61K 31/506; A61K 31/5365; A61K 31/5383; C07D 213/75; C07D 213/85; C07D 405/14; C07D 471/04; C07D 471/08; C07D 471/22; C07D 487/04; C07D 491/052; C07D 493/08; C07D 498/04; C07D 498/18

(52) U.S. Cl. CPC C07D 401/14; A61K 31/444; A61K 31/4545; A61K 31/4709; A61K 31/473; A61K 31/497; A61K 31/4985; A61K 31/501; A61K 31/506; A61K 31/5365; A61K 31/5383; C07D 213/75; C07D 213/85; C07D 405/14; C07D 471/04; C07D 471/08; C07D 471/22; C07D 487/04; C07D 491/052; C07D 493/08; C07D 498/04; C07D 498/18

(57) ABSTRACT

The present disclosure provides compounds of formula I, compositions thereof, and methods of using the same for the inhibition of SIK2 and the treatment of SIK2-mediated disorders.

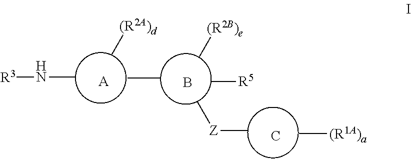

### SEQUENCE LISTING

This application contains a Sequence Listing which has been submitted in .xml format via EFS and is hereby incorporated by reference. The ST.26 copy, created on Feb. 5, 2026, is named 103-SK-P3.xml and is 1,992 bytes in size.

### TECHNICAL FIELD

The present disclosure relates to compounds and methods useful for inhibiting salt-inducible kinase 2 (SIK2). The disclosure also provides pharmaceutically acceptable compositions comprising compounds of the present disclosure and methods of using said compositions in the treatment of various disorders.

### BACKGROUND

A protein kinase is an enzyme that catalyzes the transfer of phosphate groups to proteins or other organic molecules. Consequently, defective control of protein phosphorylation leads to uncontrolled signaling is involved in a number of diseases, including for example, inflammation, allergies, cancer, autoimmune diseases, CNS disorders, and angiogenesis.

The salt-inducible kinases (SIKs) are Ser/Thr kinases members of the adenosine monophosphate-activated kinase (AMPK) subfamily of kinases and three isoforms have been described, i.e., SIK1, SIK2 (QIK), and SIK3 (QSK). The SIKs play a number of roles in different cell types have been found to phosphorylate a number of substrates including CREB-responsive transcriptional co-activator (CRTC) proteins, and also histone de-acetylase (HDAC) proteins, thereby regulating the transcription of a number of different genes.

For example, SIK2 is known to play a role in the secretion of high levels of anti-inflammatory cytokines such as interleukin-10 (IL-10), very low levels of pro-inflammatory cytokines such as TNF-α, and in the regulation of IFNγ and IL-12 signaling, suggesting SIK2 may be an interesting target for inflammatory diseases. See e.g., Darling, et al, Biochem. J. 2017, 474(4):521-537 and Yao, et al., Nat. comm. 2013, 4(1):1-15.

The lack of selectivity towards salt inducible kinase isoforms, and in particular towards SIK2 may be a possible limitation in the use of such compounds. The aim of the present disclosure is to provide new compounds which are potent SIK2 inhibitors, and which also show selectivity over SIK1 and SIK3. There is a need for selective small molecule kinase inhibitors that inhibit SIK2.

### SUMMARY

It has now been found that compounds as disclosed herein, and pharmaceutically acceptable compositions thereof, are effective as inhibitors of SIK2. In certain embodiments, provided are compounds of the formulae presented herein.

In one aspect, the disclosure provides a compound of formula I:

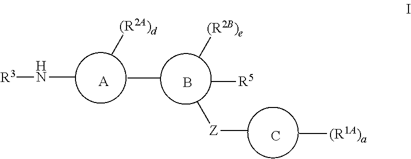

or a pharmaceutically acceptable salt thereof, wherein Z, R^{1A}, R^{2A}, R^{2B}, R³, R⁵, a, d, e, Ring A, Ring B, and Ring C, are as described and defined herein.

In some cases, the disclosure provides a compound of formula I-a-1 or I-b′:

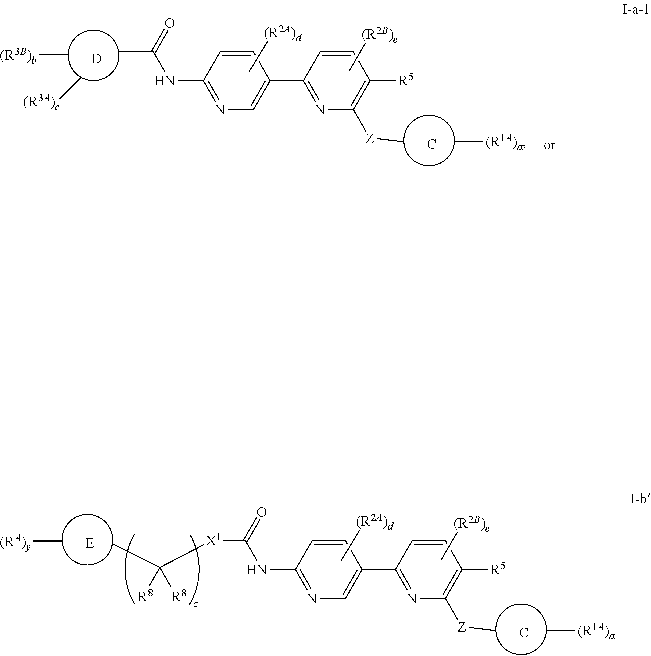

or a pharmaceutically acceptable salt thereof, wherein Z, R^{1A}, R^{2A}, R^{2B}, R^{3A}, R^{3B}, R⁵, R⁸, R^{A}, X¹, a, b, c, d, e, y, z, Ring C, Ring D, and Ring E, are as described and defined herein.

Compounds disclosed herein, and pharmaceutically acceptable compositions thereof, are useful for treating a variety of diseases, disorders or conditions, associated with modulating SIK2. Such diseases, disorders, or conditions include those described herein. Compounds provided are also useful for the study of SIK2 kinase in biological and pathological phenomena; the study of intracellular signal transduction pathways occurring in bodily tissues; and the comparative evaluation of new SIK2 inhibitors or other regulators of kinases, signaling pathways, and cytokine levels in vitro or in vivo.

### DETAILED DESCRIPTION

### 1. General Description

In certain aspects, provided herein is a compound of formula I:

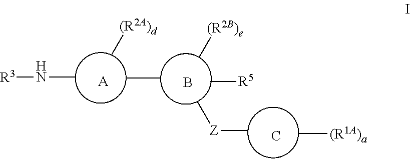

or a pharmaceutically acceptable salt thereof, wherein:
Ring A is a 6-membered heteroaryl ring having 1-3 nitrogen atoms;
Ring B is phenyl or a 5-6 membered heteroaryl ring having 1-3 heteroatoms independently selected from nitrogen, oxygen, and sulfur;
R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂ and halogen;
each R⁴ is independently selected from hydrogen and C₁₋₄ alkyl;
R³ is —C(O)-L₆, wherein L₆ is selected from the group consisting of:

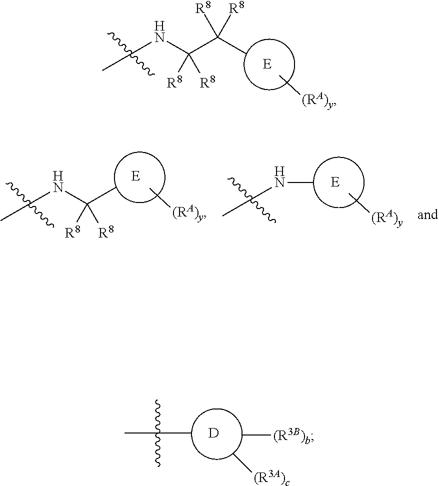

each R⁸ is independently selected from hydrogen, halogen, hydroxyl, C₁₋₃ haloalkyl, —CN, C₁₋₄ alkoxy, a 3-8 membered saturated or partially unsaturated monocyclic carbocyclic, and C₁₋₄ alkyl; or two R⁸ groups and the carbon atom to which they are attached form oxo, a spiro 3-8 membered saturated or partially unsaturated monocyclic carbocyclic ring, or a spiro 3-8 membered saturated or partially unsaturated monocyclic heterocyclic ring having 1-4 heteroatoms independently selected from nitrogen, oxygen, and sulfur, wherein said spiro 3-8 membered saturated or partially unsaturated monocyclic carbocyclic ring or spiro 3-8 membered saturated or partially unsaturated monocyclic heterocyclic ring is substituted with 0, 1 or 2 substituents independently selected from halogen, C₁₋₃ alkyl, C₁₋₃ alkoxy, and C₁₋₃ haloalkyl;
R⁵ is selected from hydrogen, —CN, OH, C₁₋₄alkyl, C₁₋₄alkoxy, C₁₋₄haloalkyl, C₁₋₄ hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄ hydroxyalkyl is optionally substituted with —CN —OH, or 1, 2, or 3 halogens, or
R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S} substituents;
each R^{S} is independently OH, C₁₋₃ alkyl, C₁₋₃ alkoxy, C₁₋₃ haloalkyl, C₁₋₃ hydroxyalkyl, —N(R⁴)₂, and halogen;
each R^{1A} is independently halogen, —CN, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or R^{1A} is a C₀₋₃alkylene-Cyc, and Cyc is selected from phenyl; 3-7 membered saturated or partially unsaturated monocyclic carbocyclic ring; 3-7 membered saturated or partially unsaturated monocyclic heterocyclic ring having 1-2 heteroatoms independently selected from nitrogen, oxygen, and sulfur; 4-12 membered saturated or partially unsaturated carbocyclic ring system that is fused, bridged, or spirocyclic, and 4-12 membered saturated or partially unsaturated heterocyclic ring system that is fused, bridged, or spirocyclic (having 1-4 heteroatoms independently selected from nitrogen, oxygen, and sulfur), wherein said Cyc is substituted with 0, 1, 2, or 3 substituents independently selected C₁₋₃ alkyl, C₁₋₃ alkoxy, C₁₋₃ haloalkyl, C₁₋₃ hydroxyalkyl, halogen, —CN, —NR₂, and a 5-6 membered heteroaryl ring having 1-4 heteroatoms independently selected from nitrogen, oxygen, and sulfur; or
   R^{1A} and R^{2A} combine to form a linker having a structure of —OC₂₋₆alkylene-NH— or —C₂₋₆alkylene-NH—, wherein the N forms a direct bond to Ring A;
   each R⁴ is independently selected from hydrogen and C₁₋₄alkyl;
Ring D is a carbon linked cyclic group selected from:
   a 3-8 membered saturated or partially unsaturated monocyclic carbocyclic ring;
   a 5-10 membered saturated or partially unsaturated spirocyclic ring having 0-3 heteroatoms independently selected from nitrogen, oxygen, and sulfur;
   a 3-8 membered saturated or partially unsaturated monocyclic heterocyclic ring having 1-2 heteroatoms independently selected from nitrogen, oxygen, and sulfur; and
   a 5-12 membered saturated or partially unsaturated bicyclic heterocyclic ring that is optionally bridged or spirocyclic having 1-4 heteroatoms independently selected from nitrogen, oxygen, and sulfur;
Ring E is a cyclic group selected from:
   a 5-6 membered monocyclic heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
y is 0, 1, 2, 3, or 4;
Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, provided that # is a direct bond to Ring B;
Ring C is a cyclic group selected from:
   phenyl;
   a 3-8 membered saturated or partially unsaturated carbocyclic ring;
   a 3-8 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 5-10 membered saturated or partially unsaturated spirocyclic ring having 0-3 heteroatoms independently selected from nitrogen, oxygen, and sulfur; and
   a 5-11 membered saturated or partially unsaturated bicyclic ring having 0-2 heteroatoms independently selected from nitrogen, oxygen, and sulfur; or
Ring C is absent and (i) a is 0 and R^{2A} and Z together form —OC₂₋₆alkylen-NH—, wherein O is the Z point of attachment to the pyridine ring; or (ii) Z is a bond to R^{1A}, and a is 1;
b is 0 or 1;
R^{3B} is halogen, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, or
R^{3B} is a 3-7 membered saturated or partially unsaturated monocyclic or bridged carbocyclic ring; a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents;
c is 0 or 1;
R^{3A} is a C₀₋₆alkylene-cyclic group; wherein the cyclic group is selected from:
   a 5-6 membered heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic or tricyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
wherein R^{3A} is substituted with 0, 1, 2, 3, or 4 R^{A};
y is 1, 2, 3 or 4;
each R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N(R)OR; —OC(O)R; —OC(O)NR₂; —N(R)CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; a fused, bridged, or spiro 4-12 membered saturated or partially unsaturated carbocyclic ring and a fused, bridged, or spiro 4-12 membered saturated or partially unsaturated heterocyclic ring system having 1-4 heteroatoms independently selected from nitrogen, oxygen, and sulfur, wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or
   two R^{A} on the same carbon atom together form oxo or spiro a cyclic group selected from:
      3-8 membered saturated or partially unsaturated monocyclic carbocyclic ring; and
      a 3-8 membered saturated or partially unsaturated monocyclic heterocyclic ring having 1-2 heteroatoms independently selected from nitrogen, oxygen, and sulfur;
wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR; halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂; —N(R)C(O)OR; —N(R)C(O)R; —N®C(O)NR₂; —N®C(NR)NR₂; —N®S(O)₂NR₂; and —N®S(O)₂R; or
   two R^{A} on adjacent atoms form a fused cyclic group selected from:
      3-8 membered saturated or partially unsaturated monocyclic carbocyclic ring;
      a 3-8 membered saturated or partially unsaturated monocyclic heterocyclic ring having 1-2 heteroatoms independently selected from nitrogen, oxygen, and sulfur;
      phenyl; and
      a 5-6 membered heteroaryl ring having 1-4 heteroatoms independently selected from nitrogen, oxygen, and sulfur;
wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR halogen; —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N®OR; —OC(O)R; —OC(O)NR₂; —N®C(O)OR; —N®C(O)R; —N®C(O)NR₂; —N®C(NR)NR₂; —N®S(O)₂NR₂; and —N®S(O)₂R;
each R⁶ is independently selected from hydrogen, halogen, C₁₋₄ alkyl, and C₁₋₄ haloalkyl, or two R⁶ combine to form oxo;
each R⁷ is independently selected from hydrogen, halogen, and C₁₋₄ alkyl, or two R⁷ combine to form oxo;
each R⁹ is independently selected from hydrogen and C₁₋₄ alkyl;
each R is independently hydrogen, C₁₋₃ alkyl, C₂₋₆ alkenyl, C₂₋₃ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, or an optionally substituted group selected from a C₁₋₆ aliphatic; cyclic group; and C₁₋₆ alkyl-cyclic group, wherein the cyclic group is selected from:
   phenyl;
   a 3-7 membered saturated or partially unsaturated carbocyclic ring;
   a 3-7 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-4 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 6-10 membered saturated or partially unsaturated spirocyclic ring having 0-3 heteroatoms independently selected from nitrogen, oxygen, and sulfur; and
   a 6-11 membered saturated or partially unsaturated bicyclic ring having 0-2 heteroatoms independently selected from nitrogen, oxygen, and sulfur;
wherein the cyclic group is substituted with 0, 1, 2, or 3 R^{S};
or
two R on the same atom together with the atom on which they are attached form a cyclic group selected from:
   a 4-7 membered saturated or partially unsaturated carbocycyl;
   a 4-7 membered saturated or partially unsaturated heterocyclyl having 1-3 heteroatoms independently selected from nitrogen, oxygen, and sulfur; and
   a 5-6 membered heteroaryl ring having 1-3 heteroatoms independently selected from nitrogen, oxygen, and sulfur,
and the cyclic group is substituted with 0, 1, 2, or 3 R^{S}.

In certain other aspects, the present disclosure provides a compound of formula I, wherein said compound is of formula I-a, I-b, I-c, or I-d:

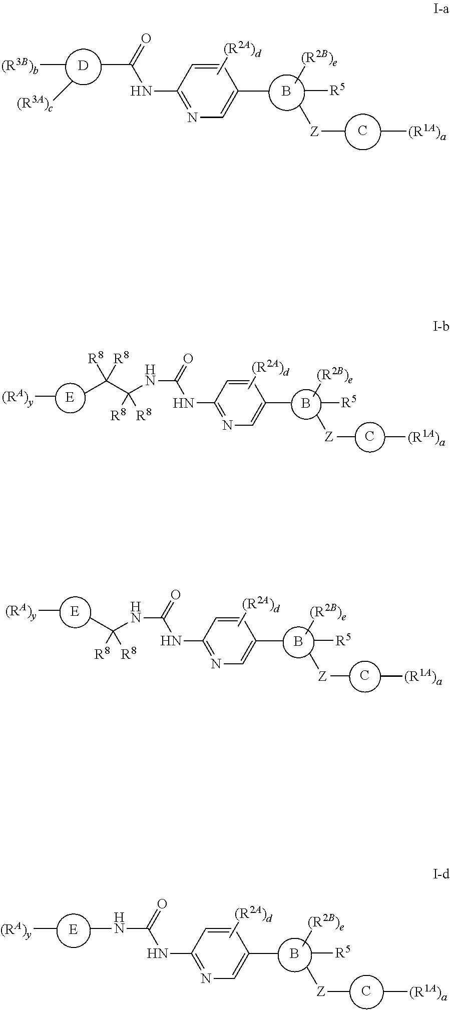

or a pharmaceutically acceptable salt thereof, wherein:
Ring B is phenyl or a 6-membered heteroaryl ring having 1-3 heteroatoms independently selected from nitrogen, oxygen, and sulfur;
R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂, and halogen;
each R⁴ is independently selected from hydrogen and C₁₋₄ alkyl;
z is 0, 1, or 2;
each R⁸ is independently selected from hydrogen, halogen, hydroxyl, C₁₋₃ haloalkyl, —CN, C₁₋₄ alkoxy, a 3-8 membered saturated or partially unsaturated monocyclic carbocyclic, and C₁₋₄ alkyl; or two R⁸ groups and the carbon atom to which they are attached form oxo, a spiro 3-8 membered saturated or partially unsaturated monocyclic carbocyclic ring, or a spiro 3-8 membered saturated or partially unsaturated monocyclic heterocyclic ring having 1-4 heteroatoms independently selected from nitrogen, oxygen, and sulfur, wherein said spiro 3-8 membered saturated or partially unsaturated monocyclic carbocyclic ring or spiro 3-8 membered saturated or partially unsaturated monocyclic heterocyclic ring is substituted with 0, 1 or 2 substituents independently selected from halogen, C₁₋₃ alkyl, C₁₋₃ alkoxy, and C₁₋₃ haloalkyl;
R⁵ is selected from hydrogen, —CN, OH, C₁₋₄alkyl, C₁₋₄alkoxy, C₁₋₄haloalkyl, C₁₋₄ hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄ hydroxyalkyl is optionally substituted with —CN —OH, or 1, 2, or 3 halogens, or
R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S} substituents;
each R^{S} is independently OH, C₁₋₃ alkyl, C₁₋₃ alkoxy, C₁₋₃ haloalkyl, C₁₋₃ hydroxyalkyl, —N(R⁴)₂, and halogen;
each R^{1A} is independently halogen, —CN, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N®OR, —OC(O)R, —OC(O)NR₂, —N®CO₂R, —N®C(O)R, —N®C(O)NR₂, —N®C(NR)R, —N®C(NR)NR₂, —N®NR₂, —N®SO₂NR₂, —N®SO₂R, —N═S(O)R₂, —S(NR)(O)R, —N®S(O)R, —N®CN, —P(O)(OR)₂, —P(O)R₂, or R^{1A} is a C₀₋₃alkylene-Cyc, and Cyc is selected from phenyl; 3-7 membered saturated or partially unsaturated monocyclic carbocyclic ring; 3-7 membered saturated or partially unsaturated monocyclic heterocyclic ring having 1-2 heteroatoms independently selected from nitrogen, oxygen, and sulfur; 4-12 membered saturated or partially unsaturated carbocyclic ring system that is fused, bridged, or spirocyclic, and 4-12 membered saturated or partially unsaturated heterocyclic ring system that is fused, bridged, or spirocyclic (having 1-4 heteroatoms independently selected from nitrogen, oxygen, and sulfur), wherein said Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —NR₂, and a 5-6 membered heteroaryl ring having 1-4 heteroatoms independently selected from nitrogen, oxygen, and sulfur; or
   R^{1A} and R^{2A} combine to form a linker having a structure of —OC₂₋₆alkylene-NH— or —C₂₋₆alkylene-NH—, wherein the N forms a direct bond to the pyridine ring;
each R is independently hydrogen, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, and C₀₋₆ alkylene-cyclic group, wherein the cyclic group is selected from:
   phenyl;
   a 3-7 membered saturated or partially unsaturated carbocyclic ring;
   a 3-7 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-4 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 6-10 membered saturated or partially unsaturated spirocyclic ring having 0-3 heteroatoms independently selected from nitrogen, oxygen, and sulfur;
   and 6-11 membered saturated or partially unsaturated bicyclic ring having 0-2 heteroatoms independently selected from nitrogen, oxygen, and sulfur;
wherein the cyclic group is substituted with 0, 1, 2, or 3 R^{S};
   or
two R on the same atom together with the atom on which they are attached form a cyclic group selected from:
   a 4-7 membered saturated or partially unsaturated carbocycyl;
   a 4-7 membered saturated or partially unsaturated heterocyclyl having 1-3 heteroatoms independently selected from nitrogen, oxygen, and sulfur; and
   a 5-6 membered heteroaryl ring having 1-3 heteroatoms independently selected from nitrogen, oxygen, and sulfur,
and the cyclic group is substituted with 0, 1, 2, or 3 R^{S};
Ring D is a carbon linked cyclic group selected from:
   a 3-8 membered saturated or partially unsaturated monocyclic carbocyclic ring;
   a 5-10 membered saturated or partially unsaturated spirocyclic ring having 0-3 heteroatoms independently selected from nitrogen, oxygen, and sulfur;
   a 3-8 membered saturated or partially unsaturated monocyclic heterocyclic ring having 1-2 heteroatoms independently selected from nitrogen, oxygen, and sulfur; and
   a 5-12 membered saturated or partially unsaturated bicyclic heterocyclic ring that is optionally bridged or spirocyclic having 1-4 heteroatoms independently selected from nitrogen, oxygen, and sulfur;
Ring E is a cyclic group selected from:
   a 5-6 membered monocyclic heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
y is 0, 1, 2, 3, or 4; Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, provided that # is a direct bond to Ring B;
Ring C is a cyclic group selected from:
   phenyl;
   a 3-8 membered saturated or partially unsaturated carbocyclic ring;
   a 3-8 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   a 8-10 membered bicyclic heteroaryl ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 5-10 membered saturated or partially unsaturated spirocyclic ring having 0-3 heteroatoms independently selected from nitrogen, oxygen, and sulfur; and
   a 5-11 membered saturated or partially unsaturated bicyclic ring having 0-2 heteroatoms independently selected from nitrogen, oxygen, and sulfur; or
Ring C is absent and (i) a is 0 and R^{2A} and Z together form —OC₂₋₆alkylen-NH—, wherein O is the Z point of attachment to the pyridine ring; or (ii) Z is a bond to R^{1A}, and a is 1;
b is 0 or 1;
R^{3B} is halogen, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N®OR, —OC(O)R, —OC(O)NR₂, —N®CO₂R, or
R^{3B} is a 3-7 membered saturated or partially unsaturated monocyclic or bridged carbocyclic ring; a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents;
c is 0 or 1;
R^{3A} is a C₀₋₆alkylene-cyclic group; wherein the cyclic group is selected from:
   a 5-6 membered heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic or tricyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
      wherein R^{3A} is substituted with 0, 1, 2, 3, or 4 R^{A};
y is 1, 2, 3 or 4;
each R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N®OR; —OC(O)R; —OC(O)NR₂; —N®CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; a fused, bridged, or spiro 4-12 membered saturated or partially unsaturated carbocyclic ring and a fused, bridged, or spiro 4-12 membered saturated or partially unsaturated heterocyclic ring system having 1-4 heteroatoms independently selected from nitrogen, oxygen, and sulfur, wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or
   two R^{A} on the same carbon atom together form oxo or spiro a cyclic group selected from:
      3-8 membered saturated or partially unsaturated monocyclic carbocyclic ring; and
      a 3-8 membered saturated or partially unsaturated monocyclic heterocyclic ring having 1-2 heteroatoms independently selected from nitrogen, oxygen, and sulfur;
wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR; halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
   two R^{A} on adjacent atoms form a fused cyclic group selected from:
   3-8 membered saturated or partially unsaturated monocyclic carbocyclic ring;
   a 3-8 membered saturated or partially unsaturated monocyclic heterocyclic ring having 1-2 heteroatoms independently selected from nitrogen, oxygen, and sulfur;
   phenyl; and
   a 5-6 membered heteroaryl ring having 1-4 heteroatoms independently selected from nitrogen, oxygen, and sulfur;
wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂;
   —N(R)C(O)OR; —N(R)C(O)R; —N(R)C(O)NR₂; —N(R)C(NR)NR₂; —N(R)S(O)₂NR₂; and —N(R)S(O)₂R;
each R⁶ is independently selected from hydrogen, halogen, C₁₋₄ alkyl, and C₁₋₄ haloalkyl, or two R⁶ combine to form oxo;
each R⁷ is independently selected from hydrogen, halogen, and C₁₋₄ alkyl, or two R⁷ combine to form oxo;
each R⁹ is independently selected from hydrogen and C₁₋₄ alkyl;

In certain other aspects, the present disclosure provides a compound of formula I, wherein said compound is of formula I-a-1, or I-b′, wherein specific subformulae of I-b′ are I-b-1, I-c-1, or I-d-1:

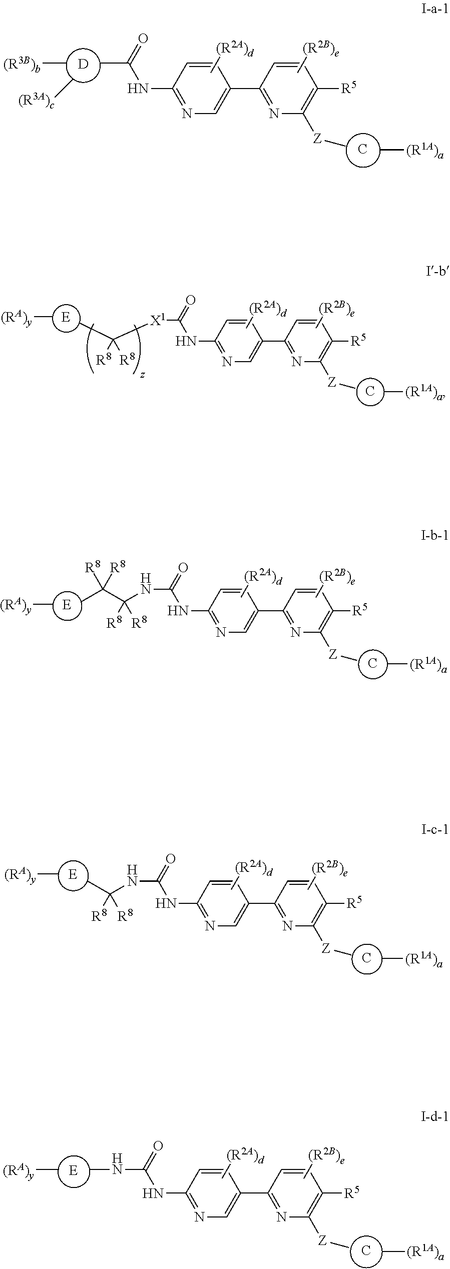

or a pharmaceutically acceptable salt thereof, wherein each of Z, X¹, R^{A}, R^{1A}, R^{2A}, R^{2B}, R⁵, a, d, e, y, z, Ring C, and Ring E, are as defined herein, both singly and in combination.

In some embodiments, the disclosure provides compounds of formula I-b′:

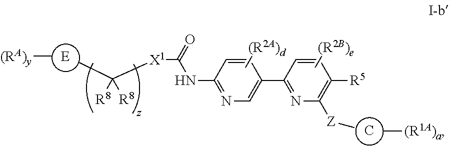

or a pharmaceutically acceptable salt thereof, wherein Z, R^{A}, R^{1A}, R^{2A}, R^{2B}, R⁵, X¹, R⁸, a, d, e, y, z, Ring C, and Ring E, are as defined herein, both singly and in combination.

In some embodiments, the present disclosure provides a pharmaceutical composition comprising a compound of formula I, formula I-a, formula I-b, formula I-b′, formula I-c, formula I-d, formula I-a-1, formula I-b-1, formula I-c-1, formula I-d-1, or a pharmaceutically acceptable salt thereof, or a pharmaceutically acceptable carrier, adjuvant, or diluent.

In some embodiments, the present disclosure provides a method of treating a SIK2-mediated disease, disorder, or condition comprising administering to a patient in need thereof, a compound of formula I, formula I-a, formula I-b, formula I-b′, formula I-c, formula I-d, formula I-a-1, formula I-b-1, formula I-c-1, formula I-d-1, or a pharmaceutically acceptable salt thereof, or a pharmaceutically acceptable carrier, adjuvant, or diluent.

In some embodiments, the disclosure provides a compound having a structure of formula I-a-1, or a pharmaceutically acceptable salt thereof. In some embodiments, the disclosure provides a compound having a structure of formula I-a-1, or a pharmaceutically acceptable salt thereof, wherein Ring C is a 5-membered heteroaryl having 1-3 heteroatoms independently selected from nitrogen, oxygen, and sulfur.

In some embodiments, the disclosure provides a compound having a structure of Formula I-b-1, I-c-1, or I-d-1, or a pharmaceutically acceptable salt thereof.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Compound structures shown throughout the present specification and in the examples or claims contain designations at certain stereocenters which indicate the following: Stereocenters marked with “abs” intend to cover material wherein the marked stereocenter is of the stereochemistry shown in the diagram. as in compound I-5.

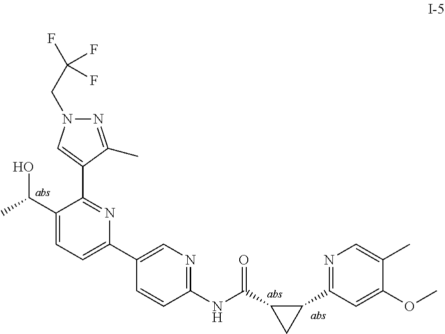 [SMILES: COc1cc([C@@H]2C[C@@H]2C(=O)Nc2ccc(-c3ccc([C@H](C)O)c(-c4cn(CC(F)(F)F)nc4C)n3)cn2)ncc1C]

Stereocenters marked with “or1” is intended to cover stereochemically pure compounds wherein the stereochemistry at the stereocenter marked with “or1” is either the stereochemistry shown in the diagram or wherein the marked stereocenter has a configuration opposite to what is shown in the diagram as in compound I-1. In structures with stereocenters with the same label such as “or1” the relative stereochemistry between two stereocenters with said label is as drawn.

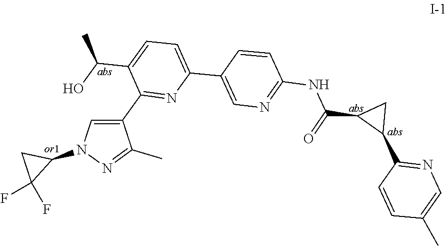 [SMILES: Cc1ccc([C@@H]2C[C@@H]2C(=O)Nc2ccc(-c3ccc([C@H](C)O)c(-c4cn([C@@H]5CC5(F)F)nc4C)n3)cn2)nc1]

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-12 carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur Unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl, ethyl, propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon-carbon double bond. In certain instances, alkenyl refers to a C₂₋₈ or a C₂₋₆ straight or branched partially unsaturated aliphatic group comprising at least one unsaturated carbon-carbon double bond. The term “lower alkenyl” refers to a C₂₋₄ straight or branched partially unsaturated aliphatic group comprising at least one unsaturated carbon-carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CHCH₂CH₃).

The term “alkynyl” refers to a C₂₋₁₂ straight or branched partially unsaturated aliphatic group comprising at least one unsaturated carbon-carbon triple bond. In certain instances, alkynyl refers to a C₂₋₈ or a C₂₋₆ straight or branched partially unsaturated aliphatic group comprising at least one unsaturated carbon-carbon triple bond. The term “lower alkynyl” refers to a C₂₋₄ straight or branched partially unsaturated aliphatic group comprising at least one unsaturated carbon-carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent straight or branched alkyl group. E.g., an “alkylene chain” can be a polymethylene group, e.g., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14π electrons shared in a cyclic array; and having, in addition to carbon atoms, from one to five ring heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3triazolyl), 1,2,4triazolyl, 1,2,5triazolyl, 1,3,4triazolyl, tetrazolyl, oxazolyl, isoxazolyl, oxadiazolyl, 1,2,3oxadiazolyl, 1,2,4oxadiazolyl, 1,2,5-oxadiazolyl, 1,3,4oxadiazolyl, thiazolyl, isothiazolyl, thiadiazolyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-only, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic.

The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent.

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; (CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(0)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄OP(O)R^{∘}₂; —(CH₂)₀₋₄OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-12-membered saturated, partially unsaturated, monocyclic, bicyclic, bridged bicyclic, or spirocyclic, or aryl mono- or bicyclic ring having 0-4 heteroatoms independently selected from nitrogen, oxygen, or sulfur, which may be substituted as defined below, or, notwithstanding the definition above, two independent occurrences of R^{∘}, taken together with their intervening atom(s), form a 3-12-membered saturated, partially unsaturated, monocyclic, bicyclic, bridged bicyclic, or spirocyclic, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)o ₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO², —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R^{●}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R^{●}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●}include halogen, —R^{●}, (haloR^{●}), OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{●}₂, or —NO², wherein each R^{●}is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In some embodiments, the compounds of the disclosure are provided in the form a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19.

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this disclosure. Such compounds are useful, for example, as analytical tools, as probes in biological assays, or as therapeutic agents in accordance with the present disclosure.

The structures as drawn represent absolute configurations unless indicated otherwise. The disclosure contemplates individual enantiomers and diastereomers, enantiomerically enriched enantiomers and diastereomers, and racemic mixtures of enantiomers and diastereomers.

As used herein, a “SIK2 inhibitor” is a molecule that reduces, inhibits, or otherwise diminishes one or more of the biological activities of SIK2 (e.g., protein phosphorylation). Inhibition using the SIK2 inhibitor does not necessarily indicate a total elimination of the SIK2 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of SIK2 compared to an appropriate control.

A compound of the present disclosure may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a SIK2 protein kinase activity between a sample comprising a compound of the present disclosure, or composition thereof, and a SIK2 protein kinase, and an equivalent sample comprising a SIK2 protein kinase, in the absence of said compound, or composition thereof.

### 3. Description of Exemplary Embodiments

As described above, in certain embodiments, the present disclosure provides a compound of formula I:

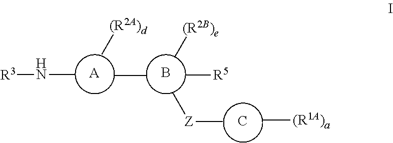

or a pharmaceutically acceptable salt thereof, wherein:
Ring A is a 6-membered heteroaryl ring having 1-3 nitrogen atoms;
Ring B is phenyl or a 5-6 membered heteroaryl ring having 1-3 heteroatoms independently selected from nitrogen, oxygen, and sulfur;
R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂ and halogen;
each R⁴ is independently selected from hydrogen and C₁₋₄ alkyl;
R³ is —C(O)-L₆, wherein L₆ is selected from the group consisting of:

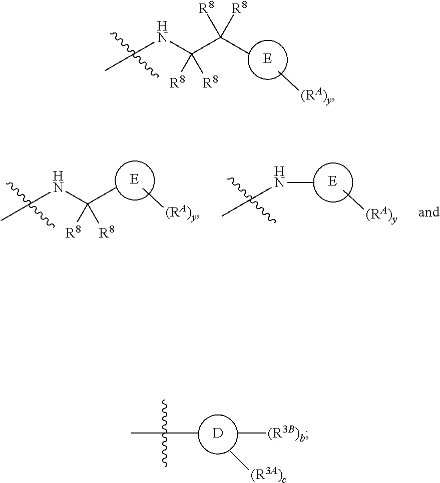

z is 0, 1, or 2;
each R⁸ is independently selected from hydrogen, halogen, hydroxyl, C₁₋₃ haloalkyl, —CN, C₁₋₄ alkoxy, a 3-8 membered saturated or partially unsaturated monocyclic carbocyclic, and C₁₋₄ alkyl; or two R⁸ groups and the carbon atom to which they are attached form oxo, a spiro 3-8 membered saturated or partially unsaturated monocyclic carbocyclic ring, or a spiro 3-8 membered saturated or partially unsaturated monocyclic heterocyclic ring having 1-4 heteroatoms independently selected from nitrogen, oxygen, and sulfur, wherein said spiro 3-8 membered saturated or partially unsaturated monocyclic carbocyclic ring or spiro 3-8 membered saturated or partially unsaturated monocyclic heterocyclic ring is substituted with 0, 1 or 2 substituents independently selected from halogen, C₁₋₃ alkyl, C₁₋₃ alkoxy, and C₁₋₃ haloalkyl;
R⁵ is selected from hydrogen, —CN, OH, C₁₋₄alkyl, C₁₋₄alkoxy, C₁₋₄haloalkyl, C₁₋₄hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄ hydroxyalkyl is optionally substituted with —CN —OH, or 1, 2, or 3 halogens, or
R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S} substituents;
each R^{S} is independently OH, C₁₋₃ alkyl, C₁₋₃ alkoxy, C₁₋₃ haloalkyl, C₁₋₃ hydroxyalkyl, —N(R⁴)₂, and halogen;
R^{1A} is C₀₋₃alkylene-Cyc and Cyc is phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 4-12 membered saturated or partially unsaturated fused, bridged, or spiro carbocyclic ring system, and a fused, bridged or spiro 4-12 membered saturated or partially unsaturated heterocyclic ring system having 1-4 heteroatoms independently selected from nitrogen, oxygen, and sulfur, and Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —NR₂; and a 5-6 membered heteroaryl ring having 1-4 heteroatoms independently selected from nitrogen, oxygen, and sulfur; or
R^{1A} and R^{2A} combine to form a linker having a structure of —OC₂₋₆alkylene-NH— or —C₂₋₆alkylene-NH—, wherein the N atom forms a direct bond with the pyridine ring;
Ring D is a carbon linked cyclic group selected from:
   a 3-8 membered saturated or partially unsaturated monocyclic carbocyclic ring;
   a 5-10 membered saturated or partially unsaturated spirocyclic ring having 0-3 heteroatoms independently selected from nitrogen, oxygen, and sulfur;
   a 3-8 membered saturated or partially unsaturated monocyclic heterocyclic ring having 1-2 heteroatoms independently selected from nitrogen, oxygen, and sulfur; and
   a 5-12 membered saturated or partially unsaturated bicyclic heterocyclic ring that is optionally bridged or spirocyclic having 1-4 heteroatoms independently selected from nitrogen, oxygen, and sulfur;
Ring E is a cyclic group selected from:
   a 5-6 membered monocyclic heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
y is 0, 1, 2, 3, or 4;
Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, provided that # is a direct bond to Ring B;
Ring C is a cyclic group selected from:
   phenyl;
   a 3-8 membered saturated or partially unsaturated carbocyclic ring;
   a 3-8 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 5-10 membered saturated or partially unsaturated spirocyclic ring having 0-3 heteroatoms independently selected from nitrogen, oxygen, and sulfur; and
   a 5-11 membered saturated or partially unsaturated bicyclic ring having 0-2 heteroatoms independently selected from nitrogen, oxygen, and sulfur; or
   Ring C is absent and (i) a is 0 and R^{2A} and Z together form —OC₂₋₆alkylen-NH—, wherein O is the Z point of attachment to the pyridine ring; or (ii) Z is a bond to R^{1A}, and a is 1;
   b is 0 or 1;
R^{3B} is halogen, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, or
R^{3B} is a 3-7 membered saturated or partially unsaturated monocyclic or bridged carbocyclic ring; a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents;
c is 0 or 1;
R^{3A} is a C₀₋₆alkylene-cyclic group; wherein the cyclic group is selected from:
   a 5-6 membered heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic or tricyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
      wherein R^{3A} is substituted with 0, 1, 2, 3, or 4 R^{A};
y is 1, 2, 3 or 4;
each R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N(R)OR; —OC(O)R; —OC(O)NR₂; —N(R)CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; a fused, bridged, or spiro 4-12 membered saturated or partially unsaturated carbocyclic ring and a fused, bridged, or spiro 4-12 membered saturated or partially unsaturated heterocyclic ring system having 1-4 heteroatoms independently selected from nitrogen, oxygen, and sulfur, wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or
   two R^{A} on the same carbon atom together form oxo or spiro a cyclic group selected from:
      3-8 membered saturated or partially unsaturated monocyclic carbocyclic ring; and
      a 3-8 membered saturated or partially unsaturated monocyclic heterocyclic ring having 1-2 heteroatoms independently selected from nitrogen, oxygen, and sulfur;
wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR; halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
   two R^{A} on adjacent atoms form a fused cyclic group selected from:
   3-8 membered saturated or partially unsaturated monocyclic carbocyclic ring;
   a 3-8 membered saturated or partially unsaturated monocyclic heterocyclic ring having 1-2 heteroatoms independently selected from nitrogen, oxygen, and sulfur;
   phenyl; and
   a 5-6 membered heteroaryl ring having 1-4 heteroatoms independently selected from nitrogen, oxygen, and sulfur;
wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR halogen; —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R⁶ is independently selected from hydrogen, halogen, C₁₋₄ alkyl, and C₁₋₄ haloalkyl, or two R⁶ combine to form oxo;
each R⁷ is independently selected from hydrogen, halogen, and C₁₋₄ alkyl, or two R⁷ combine to form oxo;
each R⁹ is independently selected from hydrogen and C₁₋₄ alkyl;
each R is independently hydrogen, C₁₋₃ alkyl, C₂₋₆ alkenyl, C₂₋₃ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, and C₀₋₆ alkylene-cyclic group, wherein the cyclic group is selected from:
   phenyl;
   a 3-7 membered saturated or partially unsaturated carbocyclic ring;
   a 3-7 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-4 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 6-10 membered saturated or partially unsaturated spirocyclic ring having 0-3 heteroatoms independently selected from nitrogen, oxygen, and sulfur; and
   a 6-11 membered saturated or partially unsaturated bicyclic ring having 0-2 heteroatoms independently selected from nitrogen, oxygen, and sulfur; and/or
wherein the cyclic group is substituted with 0, 1, 2, or 3R^{S};
or
two R on the same atom together with the atom on which they are attached form a cyclic group selected from:
   a 4-7 membered saturated or partially unsaturated carbocycyl;
   a 4-7 membered saturated or partially unsaturated heterocyclyl having 1-3 heteroatoms independently selected from nitrogen, oxygen, and sulfur; and
   a 5-6 membered heteroaryl ring having 1-3 heteroatoms independently selected from nitrogen, oxygen, and sulfur,
and the cyclic group is substituted with 0, 1, 2, or 3 R^{S}.

As described above, in certain embodiments, the present disclosure provides a compound of formula I as a compound of formula I-a, I-b, I-c, or I-d:

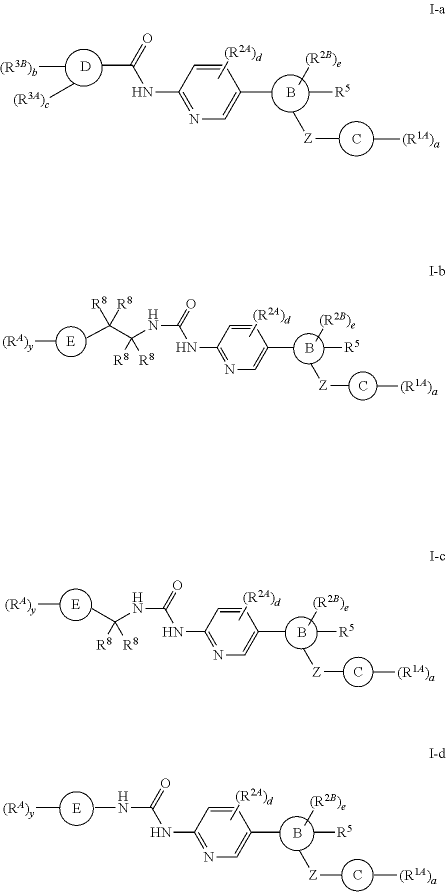

or a pharmaceutically acceptable salt thereof, wherein:
Ring B is phenyl or a 6-membered heteroaryl ring having 1-3 heteroatoms independently selected from nitrogen, oxygen, and sulfur;
R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂, and halogen;
each R⁴ is independently selected from hydrogen and C₁₋₄ alkyl;
each R⁸ is independently selected from hydrogen, halogen, hydroxyl, C₁₋₃ haloalkyl, —CN, C₁₋₄ alkoxy, a 3-8 membered saturated or partially unsaturated monocyclic carbocyclic, and C₁₋₄ alkyl; or two R⁸ groups and the carbon atom to which they are attached form oxo, a spiro 3-8 membered saturated or partially unsaturated monocyclic carbocyclic ring, or a spiro 3-8 membered saturated or partially unsaturated monocyclic heterocyclic ring having 1-4 heteroatoms independently selected from nitrogen, oxygen, and sulfur, wherein said spiro 3-8 membered saturated or partially unsaturated monocyclic carbocyclic ring or spiro 3-8 membered saturated or partially unsaturated monocyclic heterocyclic ring is substituted with 0, 1 or 2 substituents independently selected from halogen, C₁₋₃ alkyl, C₁₋₃ alkoxy, and C₁₋₃ haloalkyl;
R⁵ is selected from hydrogen, —CN, OH, C₁₋₄alkyl, C₁₋₄alkoxy, C₁₋₄haloalkyl, C₁₋₄hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄ hydroxyalkyl is optionally substituted with —CN —OH, or 1, 2, or 3 halogens, or
R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S} substituents;
each R^{S} is independently OH, C₁₋₃ alkyl, C₁₋₃ alkoxy, C₁₋₃ haloalkyl, C₁₋₃ hydroxyalkyl, —N(R⁴)₂, and halogen;
each R^{1A} is independently halogen, —CN, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N®OR, —OC(O)R, —OC(O)NR₂, —N®CO₂R, —N®C(O)R, —N®C(O)NR₂, —N®C(NR)R, —N®C(NR)NR₂, —N®NR₂, —N®SO₂NR₂, —N®SO₂R, —N═S(O)R₂, —S(NR)(O)R, —N®S(O)R, —N®CN, —P(O)(OR)₂, —P(O)R₂, or
R^{1A} is C₀₋₃alkylene-Cyc and Cyc is phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 4-12 membered saturated or partially unsaturated fused, bridged, or spiro carbocyclic ring system, and a fused, bridged or spiro 4-12 membered saturated or partially unsaturated heterocyclic ring system having 1-4 heteroatoms independently selected from nitrogen, oxygen, and sulfur, and Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —NR₂; and a 5-6 membered heteroaryl ring having 1-4 heteroatoms independently selected from nitrogen, oxygen, and sulfur; or
R^{1A} and R^{2A} combine to form a linker having a structure of —OC₂₋₆alkylene-NH— or —C₂₋₆alkylene-NH—, wherein the N atom forms a direct bond with the pyridine ring;
Ring D is a carbon linked cyclic group selected from:
   a 3-8 membered saturated or partially unsaturated monocyclic carbocyclic ring;
   a 5-10 membered saturated or partially unsaturated spirocyclic ring having 0-3 heteroatoms independently selected from nitrogen, oxygen, and sulfur;
   a 3-8 membered saturated or partially unsaturated monocyclic heterocyclic ring having 1-2 heteroatoms independently selected from nitrogen, oxygen, and sulfur; and
   a 5-12 membered saturated or partially unsaturated bicyclic heterocyclic ring that is optionally bridged or spirocyclic having 1-4 heteroatoms independently selected from nitrogen, oxygen, and sulfur;
Ring E is a cyclic group selected from:
   a 5-6 membered monocyclic heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
y is 0, 1, 2, 3, or 4;
Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, provided that # is a direct bond to Ring B;
Ring C is a cyclic group selected from:
   phenyl;
   a 3-8 membered saturated or partially unsaturated carbocyclic ring;
   a 3-8 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   a 8-10 membered bicyclic heteroaryl ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 5-10 membered saturated or partially unsaturated spirocyclic ring having 0-3 heteroatoms independently selected from nitrogen, oxygen, and sulfur; and
   a 5-11 membered saturated or partially unsaturated bicyclic ring having 0-2 heteroatoms independently selected from nitrogen, oxygen, and sulfur; or
Ring C is absent and (i) a is 0 and R^{2A} and Z together form —OC₂₋₆alkylen-NH—, wherein O is the Z point of attachment to the pyridine ring; or (ii) Z is a bond to R^{1A}, and a is 1;
b is 0 or 1;
R^{3B} is halogen, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N®OR, —OC(O)R, —OC(O)NR₂, —N®CO₂R, or
R^{3B} is a 3-7 membered saturated or partially unsaturated monocyclic or bridged carbocyclic ring; a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents;
c is 0 or 1;
   a 5-6 membered heteroaromatic ring having 1-4 heteroatoms independently selected from nitrogen, oxygen, and sulfur;
   phenyl;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur;
wherein R^{3A} is substituted with y instances of R^{A};
a is 1, 2, or 3; wherein when Z and Ring C are absent R^{1A} is not hydrogen;
b, c, d, and e are independently 0 or 1;
y is 1, 2, 3 or 4;
each R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N®OR; —OC(O)R; —OC(O)NR₂; —N®CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; a fused, bridged, or spiro 4-12 membered saturated or partially unsaturated carbocyclic ring and a fused, bridged, or spiro 4-12 membered saturated or partially unsaturated heterocyclic ring system having 1-4 heteroatoms independently selected from nitrogen, oxygen, and sulfur, wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or
   two R^{A} on the same carbon atom together form oxo or spiro a cyclic group selected from:
      3-8 membered saturated or partially unsaturated monocyclic carbocyclic ring; and
      a 3-8 membered saturated or partially unsaturated monocyclic heterocyclic ring having 1-2 heteroatoms independently selected from nitrogen, oxygen, and sulfur;
wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR; halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
   two R^{A} on adjacent atoms form a fused cyclic group selected from:
      3-8 membered saturated or partially unsaturated monocyclic carbocyclic ring;
      a 3-8 membered saturated or partially unsaturated monocyclic heterocyclic ring having 1-2 heteroatoms independently selected from nitrogen, oxygen, and sulfur;
      phenyl; and
      a 5-6 membered heteroaryl ring having 1-4 heteroatoms independently selected from nitrogen, oxygen, and sulfur;
wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR halogen; —CN; —NO²; —OR;
   —SR; —NR₂; —S(O)₂R; —S(O)₂NR₂; —S(O)R; —S(O)NR₂; —C(O)R; —C(O)OR;
   —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R⁶ is independently selected from hydrogen, halogen, C₁₋₄ alkyl, and C₁₋₄ haloalkyl, or two R⁶ combine to form oxo;
each R⁷ is independently selected from hydrogen, halogen, and C₁₋₄ alkyl, or two R⁷ combine to form oxo;
each R⁹ is independently selected from hydrogen and C₁₋₄ alkyl;
each R is independently hydrogen, C₁₋₃ alkyl, C₂₋₆ alkenyl, C₂₋₃ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, and a C₀₋₆ alkylene-cyclic group, wherein the cyclic group is selected from:
   phenyl;
   a 3-7 membered saturated or partially unsaturated carbocyclic ring;
   a 3-7 membered saturated or partially unsaturated heterocyclic ring having 1-2 heteroatoms independently selected from nitrogen, oxygen, and sulfur;
   a 5-6 membered heteroaryl ring having 1-4 heteroatoms independently selected from nitrogen, oxygen, and sulfur;
   an 8-10 membered bicyclic heteroaryl ring having 1-4 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-8 membered saturated or partially unsaturated bridged bicyclic ring having 0-3 heteroatoms independently selected from nitrogen, oxygen, and sulfur;
   a 6-10 membered saturated or partially unsaturated spirocyclic ring having 0-3 heteroatoms independently selected from nitrogen, oxygen, and sulfur;
   and 6-11 membered saturated or partially unsaturated bicyclic ring having 0-2 heteroatoms independently selected from nitrogen, oxygen, and sulfur;
wherein the cyclic group is substituted with 0, 1, 2, or 3 R^{S};
or
two R on the same atom together with the atom on which they are attached form a cyclic group selected from:
   a 4-7 membered saturated or partially unsaturated carbocycyl;
   a 4-7 membered saturated or partially unsaturated heterocyclyl having 1-3 heteroatoms independently selected from nitrogen, oxygen, and sulfur; and
   a 5-6 membered heteroaryl ring having 1-3 heteroatoms independently selected from nitrogen, oxygen, and sulfur.
and the cyclic group is substituted with 0, 1, 2, or 3 R^{S}.

In certain other aspects, the present disclosure provides a compound of formula I, wherein said compound is of formula I-a-1, I-b-1, I-c-1, or I-d-1:

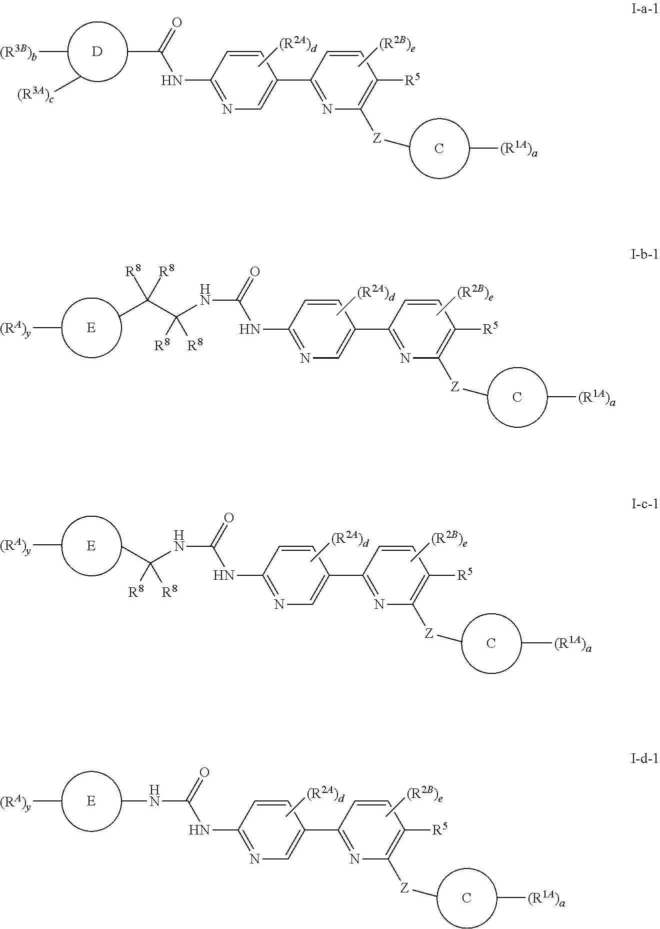

or a pharmaceutically acceptable salt thereof, wherein each of Z, R^{1A}, R^{2A}, R^{2B}, R^{3A}, R^{3B}, R^{A}, R⁵, R⁸, a, b, c, d, y, Ring C, Ring D and Ring E, are as defined herein, both singly and in combination.

In some embodiments, the present disclosure provides a compound of formula I-a-1:

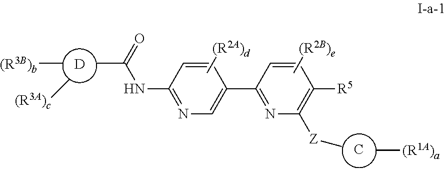

or a pharmaceutically acceptable salt thereof, wherein each of Z, R¹, R^{2A}, R^{2B}, R^{3A}, R^{3B}, R⁵, a, b, c, d, e, and Ring D, are as defined herein, both singly and in combination, and Ring C is a 5-membered heteroaryl having 1-3 heteroatoms independently selected from nitrogen, oxygen, and sulfur.

In some embodiments, the disclosure provides a compound of formula I-b′:

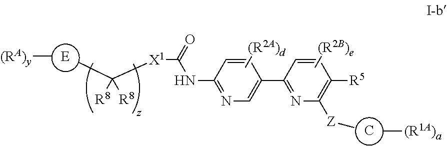

or a pharmaceutically acceptable salt thereof, wherein each of Z, R^{1A}, R^{2A}, R^{2B}, R^{A}, R⁵, R⁸, a, y, d, e, and Ring E, are as defined herein; X¹ is O or NH; z is 0, 1, or 2; both singly and in combination, and Ring C is a 5-membered heteroaryl having 1-3 heteroatoms independently selected from nitrogen, oxygen, and sulfur.

In some embodiments, X¹ is NH. In some embodiments X¹ is O.

In some embodiments, z is 0. In some embodiments, z is 1. In some embodiments, z is 2.

In some embodiments, the present disclosure provides a compound of formula I-b-1:

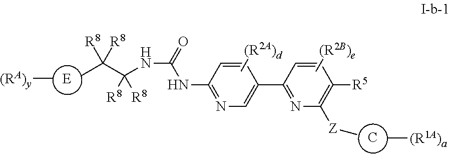

or a pharmaceutically acceptable salt thereof, wherein each of Z, R^{A}, R^{2A}, R^{2B}, R^{A}, R⁵, R⁸, a, y, d, e, and Ring E, are as defined herein, both singly and in combination, and Ring C is a 5-membered heteroaryl having 1-3 heteroatoms independently selected from nitrogen, oxygen, and sulfur.

In some embodiments, the present disclosure provides a compound of formula I-c-1:

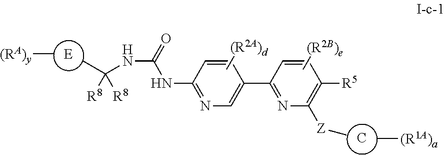

or a pharmaceutically acceptable salt thereof, wherein each of Z, R^{1A}, R^{2A}, R^{2B}, R^{A}, R⁵, R⁸, a, y, d, e, and Ring E, are as defined herein, both singly and in combination, and Ring C is a 5-membered heteroaryl having 1-3 heteroatoms independently selected from nitrogen, oxygen, and sulfur.

In some embodiments, the present disclosure provides a compound of formula I-d-1:

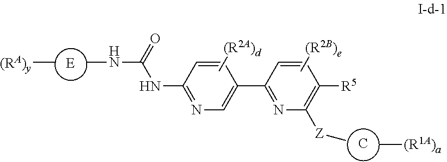

or a pharmaceutically acceptable salt thereof, wherein each of Z, R¹, R^{2A}, R^{2B}, R^{3A}, R^{3B}, R^{A}, R⁵, R⁸, a, y, d, e, and Ring E, are as defined herein, both singly and in combination, and Ring C is a 5-membered heteroaryl having 1-3 heteroatoms independently selected from nitrogen, oxygen, and sulfur.

In some embodiments, the present disclosure provides a compound of formula I-a-1:

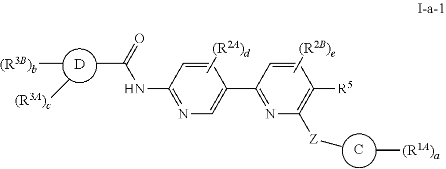

or a pharmaceutically acceptable salt thereof, wherein each of Z, R^{1A}, R^{2A}, R^{2B}, R^{3A}, R^{3B}, R⁵, a, b, c, d, e, and Ring D, are as defined herein, both singly and in combination, and Ring C is a 6 membered heteroaryl ring having 1-4 heteroatoms independently selected from nitrogen, oxygen, and sulfur. In some cases, ring D is

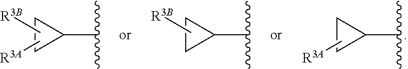

In some embodiments, the present disclosure provides a compound of formula I-b-1:

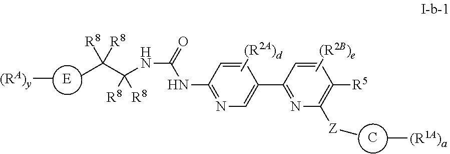

or a pharmaceutically acceptable salt thereof, wherein each of Z, R^{1A}, R^{2A}, R^{2B}, R^{A}, R⁵, R⁸, a, y, d, e, and Ring E, are as defined herein, both singly and in combination, and Ring C is a 6 membered heteroaryl ring having 1-4 heteroatoms independently selected from nitrogen, oxygen, and sulfur.

In some embodiments, the present disclosure provides a compound of formula I-a-1:

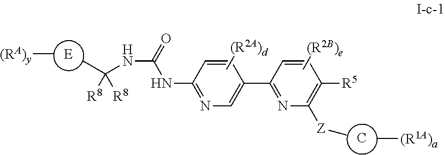

or a pharmaceutically acceptable salt thereof, wherein each of Z, R^{1A}, R^{2A}, R^{2B}, R^{A}, R⁵, R⁸, a, y, d, e, and Ring E, are as defined herein, both singly and in combination, and Ring C is a 6 membered heteroaryl ring having 1-4 heteroatoms independently selected from nitrogen, oxygen, and sulfur.

In some embodiments, the present disclosure provides a compound of formula I-a-1:

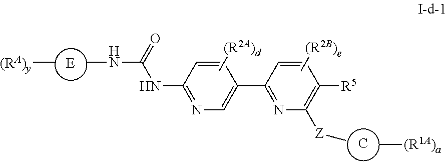

or a pharmaceutically acceptable salt thereof, wherein each of Z, R^{1A}, R^{2A}, R^{2B}, R^{3A}, R^{A}, R⁵, a, y, d, e, and Ring E, are as defined herein, both singly and in combination, and Ring C is a 6 membered heteroaryl ring having 1-4 heteroatoms independently selected from nitrogen, oxygen, and sulfur.

As defined generally above, R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂, and halogen.

In some embodiments, R^{2A} is hydrogen. In some embodiments, R^{2A} is OH. In some embodiments, R^{2A} is C₁₋₃ alkyl. In some embodiments, R^{2A} is C₁₋₃ alkoxy. In some embodiments, R^{2A} is C₁₋₃ haloalkyl. In some embodiments, R^{2A} is C₁₋₃ hydroxyalkyl. In some embodiments, R^{2A} is halogen. In some embodiments, R^{2A} is —N(R⁴)₂.

In some embodiments, R^{2A} is hydrogen, F, Cl, CH₃, OCH₃, or CH₂CH₃.

In some embodiments, R^{2A} is H. In some embodiments, R^{2A} is F. In some embodiments, R^{2A} is Cl. In some embodiments, R^{2A} is CH₃. In some embodiments, R^{2A} is OCH₃. In some embodiments, R^{2A} is CH₂CH₃. In some embodiments, R^{2A} is CH₂F. In some embodiments, R^{2A} is CHF₂. In some embodiments, R^{2A} is CF₃. In some embodiments, R^{2A} is NH₂. In some embodiments, R^{2A} is NHCH₃. In some embodiments, R^{2A} is N(CH₃)₂. In some embodiments, R^{2A}is CH₂OH. In some embodiments, R^{2A} is CH₂CH₂OH.

In some embodiments, R^{2A} is as depicted in the compounds of Table 1, below.

In some embodiments, R^{2B} is hydrogen. In some embodiments, R^{2B} is OH. In some embodiments, R^{2B} is C₁₋₃ alkyl. In some embodiments, R^{2B} is C₁₋₃ alkoxy. In some embodiments, R^{2B} is C₁₋₃ haloalkyl. In some embodiments, R^{2B} is C₁₋₃ hydroxyalkyl. In some embodiments, R^{2B} is halogen. In some embodiments, R^{2B} is —N(R⁴)₂.

In some embodiments, R^{2B} is hydrogen, F, Cl, CH₃, OCH₃, or CH₂CH₃.

In some embodiments, R^{2B} is H. In some embodiments, R^{2B} is F. In some embodiments, R^{2B} is Cl. In some embodiments, R^{2B} is CH₃. In some embodiments, R^{2B} is OCH₃. In some embodiments, R^{2B} is CH₂CH₃. In some embodiments, R^{2B} is CH₂F. In some embodiments, R^{2B} is CHF₂. In some embodiments, R^{2B} is CF₃. In some embodiments, R^{2B} is NH₂. In some embodiments, R^{2B} is NHCH₃. In some embodiments, R^{2B} is N(CH₃)₂. In some embodiments, R^{2B} is CH₂OH. In some embodiments, R^{2B} is CH₂CH₂OH.

In some embodiments, R^{2B} is as depicted in the compounds of Table 1, below.

As defined generally above, R⁵ is selected from hydrogen, —CN, OH, C₁₋₄ alkyl, C₁₋₄ alkoxy, C₁₋₄ haloalkyl, C₁₋₄hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄hydroxyalkyl is optionally substituted with —CN —OH, or 1, 2, or 3 halogens, or R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S}substituents, wherein each R^{S} is independently OH, C₁₋₃ alkyl, C₁₋₃ alkoxy, C₁₋₃ haloalkyl, C₁₋₃ hydroxyalkyl, —N(R⁴)₂, and halogen.

In some embodiments, R^{s} is hydrogen. In some embodiments, R⁵ is —CN. In some embodiments, R^{s} is OH. In some embodiments, R⁵ is C₁₋₄ alkyl. In some embodiments, R⁵ is C₁₋₄ alkoxy. In some embodiments, R⁵ is C₁₋₄haloalkyl. In some embodiments, R⁵ is C₁₋₄hydroxyalkyl.

In some embodiments, R⁵ is halogen. In some embodiments, R⁵ is —OR. In some embodiments, R⁵ is —SR. In some embodiments, R⁵ is —NR₂. In some embodiments, R⁵ is —SO₂R. In some embodiments, R⁵ is —SO₂NR₂. In some embodiments, R⁵ is —S(O)R. In some embodiments, R⁵ is —S(O)NR₂. In some embodiments, R⁵ is —C(O)R. In some embodiments, R⁵ is —CO₂R. In some embodiments, R⁵ is —C(O)NR₂. In some embodiments, R⁵ is —OC(O)R. In some embodiments, R⁵ is —OC(O)NR₂. In some embodiments, R⁵ is C₁₋₄ alkyl substituted with —CN or 1, 2, or 3 halogens.

In some embodiments, R⁵ is C₁₋₄ alkoxy substituted with —CN or 1, 2, or 3 halogens. In some embodiments, R⁵ is C₁₋₄ hydroxyalkyl substituted with —CN or 1, 2, or 3 halogens. In some embodiments, R⁵ is an optionally substituted C₁₋₆ aliphatic. In some embodiments, R⁵ is an optionally substituted 3-7 membered saturated or partially unsaturated monocyclic carbocyclic ring. In some embodiments, R⁵ is an optionally substituted 3-7 membered saturated or partially unsaturated monocyclic heterocyclic ring having 1-2 heteroatoms independently selected from nitrogen, oxygen, and sulfur. In some embodiments, R⁵ is C₁₋₄hydroxyalkyl. In some embodiments, R⁵ is CH(OH)CH₃.

In some embodiments, R⁵ is independently selected from —CN and C₁₋₄ hydroxyalkyl.

In some embodiments, R⁵ is —CN, —F, —CHF₂,

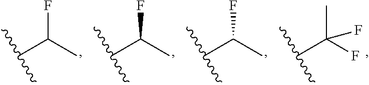

CH₂OH, —CH₂OMe, —COMe, —CO₂Me,

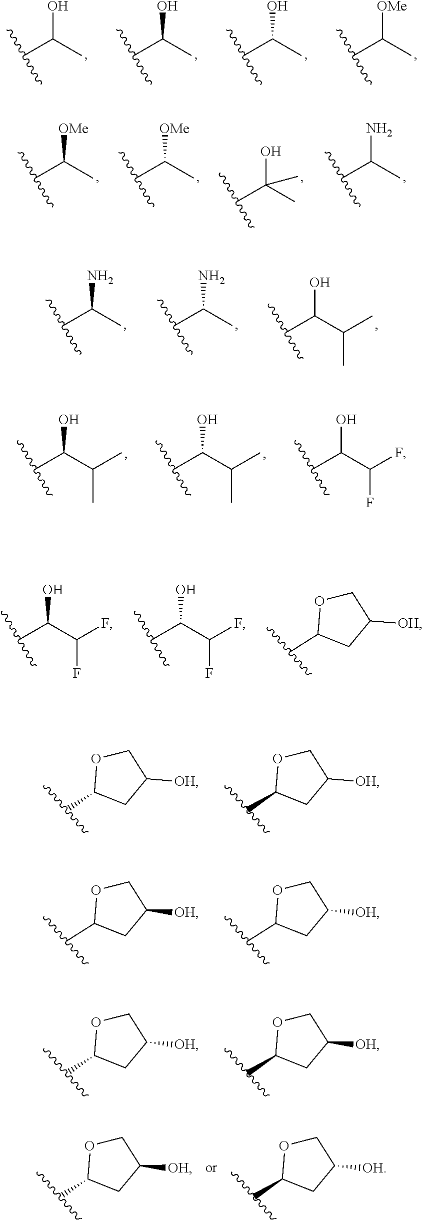

In some embodiments, R⁵ is —CN,

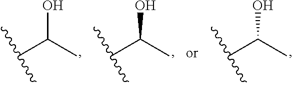

In some embodiments, R⁵ is as depicted in the compounds of Table 1, below.

As defined generally above, R⁶ is independently selected from hydrogen, halogen, C₁₋₄ alkyl, or C₁₋₄haloalkyl, or two R⁶ combine to form oxo.

In some embodiments, R⁶ is hydrogen. In some embodiments, R⁶ is C₁₋₄ alkyl. In some embodiments, R⁶ is halogen. In some embodiments, R⁶ is C₁₋₄ haloalkyl. In some embodiments, R⁶ is two R⁶ groups form oxo.

In some embodiments, R⁶ is hydrogen, F, Cl, CH₃, or two R⁶ form oxo.

In some embodiments, two R⁶ form oxo.

In some embodiments, R⁶ is F. In some embodiments, R⁶ is Cl. In some embodiments, R⁶ is CH₃.

In some embodiments, R⁶ is as depicted in the compounds of Table 1, below.

As defined generally above, Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, provided that # is a direct bond to Ring B. In some embodiments, Z is a bond, —CH₂—CH₂—, —CH₂—, —O—, #C(O)—CH₂—, #O—CH₂—, #O—CH₂—CH₂—, #CH₂—O—, #CH₂—CH₂—O—, #NH—CH₂—, #NH—CH₂—CH₂—, #N(CH₃)—CH₂—, or #N(CH₃)—CH₂—CH₂—, wherein # is a direct bond to Ring B.

In some embodiments, Z is a bond. In some embodiments, Z is #C(R⁶)₂—[C(R⁷)₂]₀₋₂—. In some embodiments, Z is #O—[C(R⁷)₂]₀₋₂—. In some embodiments, Z is #NR⁶—[C(R⁹)₂]₀₋₂—. In some embodiments, Z is #C(R⁶)₂—[C(R⁷)₂]₀₋₂—. In some embodiments, Z is #O—[C(R⁷)₂]₂—. In some embodiments, Z is #NR⁶—[C(R⁹)₂]₂—. In some embodiments, Z is #C(R⁶)₂—. In some embodiments, Z is #O—[C(R⁷)₂]—. In some embodiments, Z is #NR⁶—. In some embodiments, Z is #C(R⁶)₂—[C(R⁷)₂]—. In some embodiments, Z is #NR⁶—[C(R⁹)₂]—. In some embodiments, Z is #C(R⁶)₂—[C(R⁷)₂]₁₋₂—. In some embodiments, Z is #O—[C(R⁷)₂]₁₋₂—. In some embodiments, Z is #NR⁶—[C(R⁹)₂]₁₋₂—.

In some embodiments, Z is a bond, —CH₂—CH₂—, —CH₂—, —C(O)—CH₂—, —O—, —O—CH₂—, —O—CH₂—CH₂—, —NH—CH₂—, —NH—CH₂—CH₂—, —N(CH)₃—CH₂—, —CH₂—O—, —CH₂—CH₂—O—, —NH—, —N(CH)₃—, or —N(CH)₃—CH₂—CH₂—.

In some embodiments, Z is —CH₂—CH₂—. In some embodiments, Z is —CH₂—. In some embodiments, Z is —O—. In some embodiments, Z is —C(O)—CH₂—. In some embodiments, Z is —O—CH₂—. In some embodiments, Z is —O—CH₂—CH₂—. In some embodiments, Z is —CH₂—O—. In some embodiments, Z is —CH₂—CH₂—O—. In some embodiments, Z is —NH—CH₂—. In some embodiments, Z is —NH—. In some embodiments, Z is —NH—CH₂—CH₂—. In some embodiments, Z is —N(CH₃)—. In some embodiments, Z is —N(CH₃)—CH₂—. In some embodiments, Z is —N(CH₃)—CH₂—CH₂—.

In some embodiments, Z is as depicted in the compounds of Table 1, below.

As defined generally above, each R⁷ is independently selected from hydrogen, halogen, C₁₋₄ alkyl, or two R⁷ groups may combine to form oxo.

In some embodiments, R⁷ is hydrogen. In some embodiments, R⁷ is C₁₋₄ alkyl. In some embodiments, R⁷ is halogen. In some embodiments, two R⁷ groups form oxo.

In some embodiments, R⁷ is hydrogen, F, Cl, CH₃, or two R⁶ form oxo.

In some embodiments, two R⁷ groups form oxo.

In some embodiments, R⁷ is F. In some embodiments, R⁷ is Cl. In some embodiments, R⁷ is CH₃.

In some embodiments, R⁷ is as depicted in the compounds of Table 1, below.

As defined generally above, each R⁹ is independently selected from hydrogen and C₁₋₄ alkyl.

In some embodiments, R⁹ is hydrogen. In some embodiments, R⁹ is C₁₋₄ alkyl.

In some embodiments, R⁹ is methyl.

In some embodiments, R⁷ is as depicted in the compounds of Table 1, below.

As defined generally above, Ring A is a 6-membered heteroaryl ring having 1-3 nitrogen atoms;

In some embodiments, Ring A is

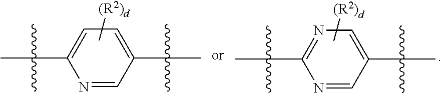

In some embodiments, Ring A is

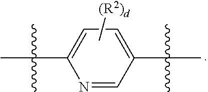

As defined generally above, Ring B is phenyl or a 5-6 membered heteroaryl ring having 1-3 heteroatoms independently selected from nitrogen, oxygen, and sulfur.

In some embodiments, Ring B and its substituents is

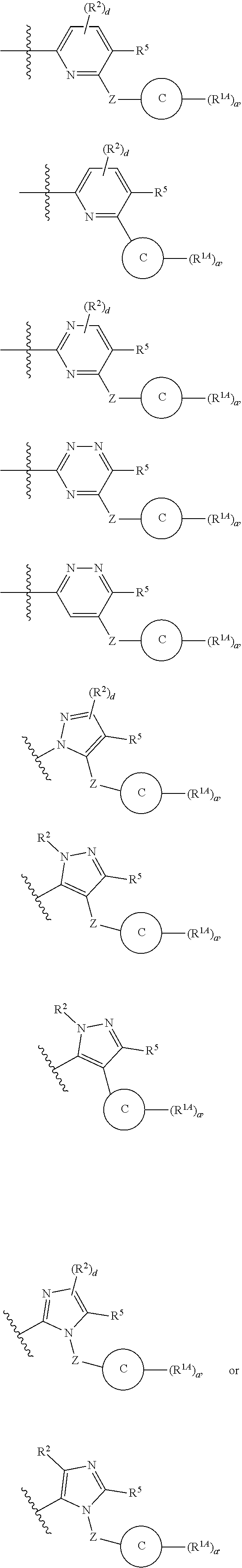

In some embodiments, Ring B and its substituents is or

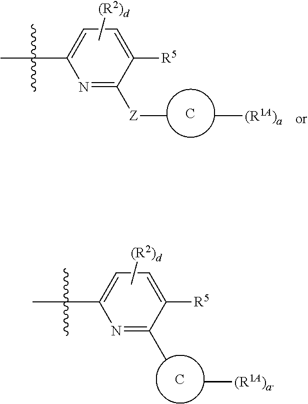

As defined generally above, Ring C is a cyclic group selected from: phenyl; a 3-8 membered saturated or partially unsaturated carbocyclic ring; a 3-8 membered saturated or partially unsaturated heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; and a 5-11 membered saturated or partially unsaturated bicyclic ring having 0-2 heteroatoms independently selected from nitrogen, oxygen, and sulfur; or Ring C is absent and (i) a is 0 and R^{2A} and Z together form —OC₂₋₆alkylen-NH—, wherein O is the Z point of attachment to the pyridine ring; or (ii) Z is a bond to R^{1A}, and a is 1; and b is 0 or 1.

In some embodiments, Ring C is a 5-6 membered heteroaryl ring having 1-3 heteroatoms independently selected from nitrogen, oxygen, and sulfur. In some embodiments, Ring C is 8-10 membered bicyclic heteroaryl ring having 1-4 heteroatoms independently selected from nitrogen, oxygen, and sulfur. In some embodiments, Ring C is a 7-12 membered saturated or partially unsaturated bicyclic heterocyclyl ring having 1-4 heteroatoms independently selected from nitrogen, oxygen, and sulfur.

In some embodiments, Ring C is

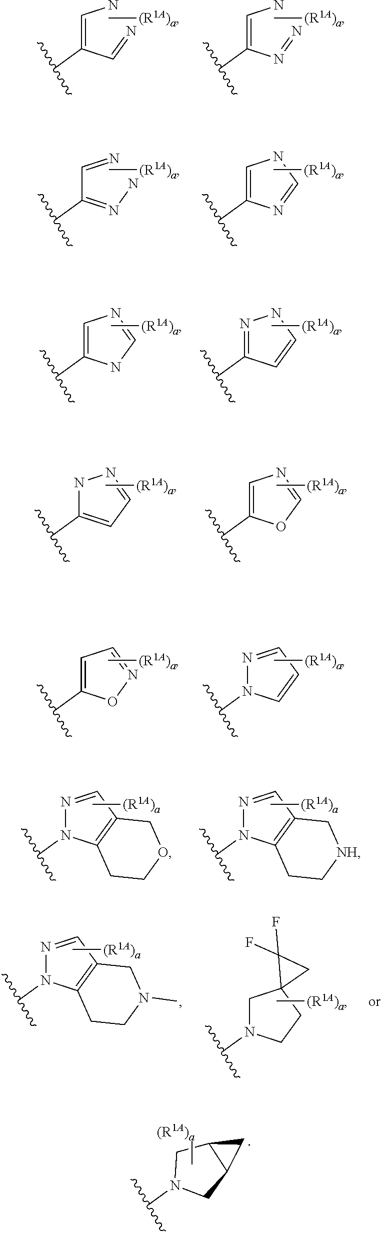

In some embodiments, Ring C is

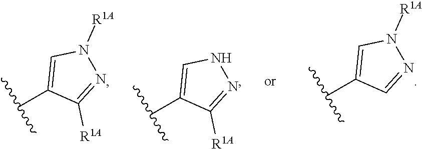

In some embodiments, Ring C is

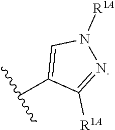

In some embodiments, Ring C is

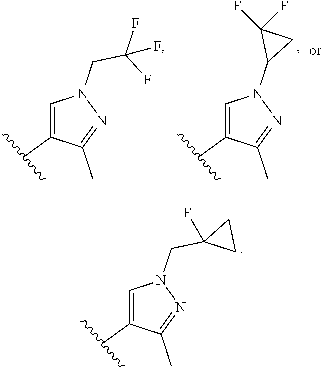 [SMILES: *c1cn(CC(F)(F)F)nc1C ; *c1cn(C2CC2(F)F)nc1C ; *c1cn(CC2(F)CC2)nc1C]

In some embodiments, Ring C is as depicted in the compounds of Table 1, below.

In some embodiments, Z-Ring C is

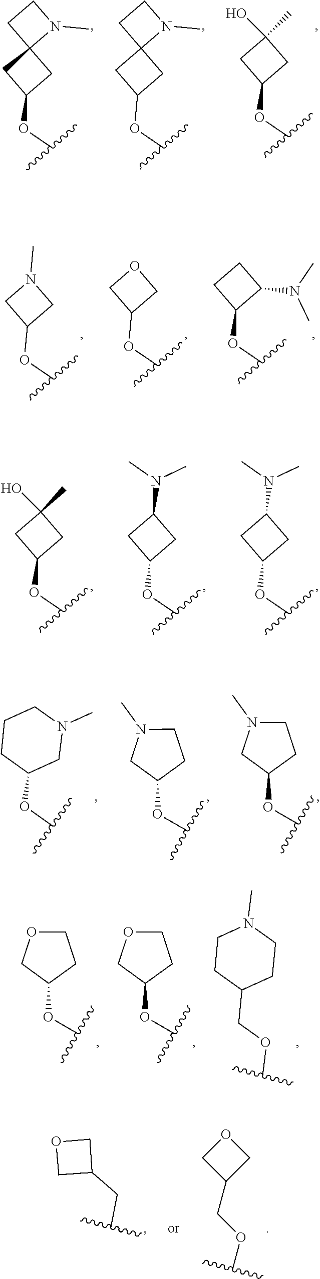

In some embodiments, Z-Ring C is as depicted in the compounds of Table 1, below.

In some embodiments, Ring C is absent and Z—R^{1A} is

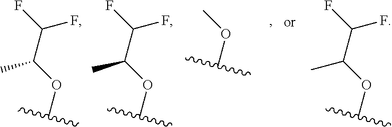

In some embodiments, Ring C is absent and R^{2A} and Z together form —OC₂₋₆alkylkene-NH—, wherein the oxygen is the Z point of attachment to the pyridine ring comprising R⁵.

In some embodiments, Z—R^{1A} is as depicted in the compounds of Table 1, below.

As defined generally above, Ring D is a carbon linked cyclic group selected from a 3-8 membered saturated or partially unsaturated monocyclic carbocyclic ring; an optionally substituted 5-10 membered saturated or partially unsaturated spirocyclic ring having 0-3 heteroatoms independently selected from nitrogen, oxygen, and sulfur; a 3-8 membered saturated or partially unsaturated monocyclic heterocyclic ring having 1-2 heteroatoms independently selected from nitrogen, oxygen, and sulfur; and a 5-12 membered saturated or partially unsaturated bicyclic heterocyclic ring that is optionally bridged or spirocyclic having 1-4 heteroatoms independently selected from nitrogen, oxygen, and sulfur.

In some embodiments, Ring D is a 3-8 membered saturated or partially unsaturated monocyclic carbocyclic. In some embodiments, Ring D is 3-8 membered saturated or partially unsaturated monocyclic heterocyclic ring having 1-2 heteroatoms independently selected from nitrogen, oxygen, and sulfur.

In some embodiments, Ring D is

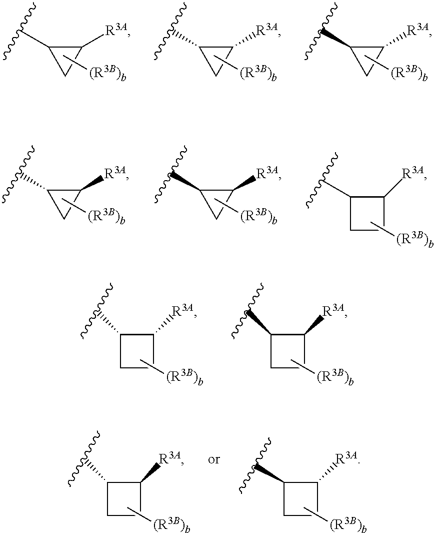

In some instances, Ring D is

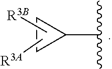

In some instances, Ring D is

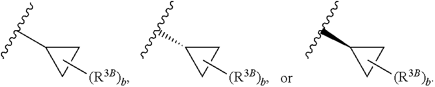

In some instances, Ring D-R^{3A} is

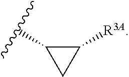

In some embodiments, Ring D is as depicted in the compounds of Table 1, below.

As defined generally above, R^{3A} is a C₀₋₆alkylene cyclic group; wherein the cyclic group is selected from phenyl; a 5-6 membered heteroaromatic ring having 1-4 heteroatoms independently selected from nitrogen, oxygen, and sulfur; a 5-6 membered heteroaromatic ring having 1-4 heteroatoms independently selected from nitrogen, oxygen, and sulfur; an 8-10 membered bicyclic heteroaromatic ring having 1-5 heteroatoms independently selected from nitrogen, oxygen, and sulfur; a 7-12 membered saturated or partially unsaturated bicyclic or tricyclic heterocyclic ring having 1-4 heteroatoms independently selected from nitrogen, oxygen, and sulfur; a 5-12 membered saturated or partially unsaturated bicyclic carbocyclic ring that is optionally bridged or spirocyclic; and a 5-12 membered saturated or partially unsaturated bicyclic heterocyclic ring that is optionally bridged or spirocyclic having 1-4 heteroatoms independently selected from nitrogen, oxygen, and sulfur; wherein R^{3A} is substituted with 0, 1, 2, 3, or 4 R^{A}.

In some embodiments, R^{3A} is a C₀₋₆alkylene cyclic group. In some embodiments, R^{3A} is phenyl. In some embodiments, R^{3A} is a 5-6 membered heteroaromatic ring having 1-4 heteroatoms independently selected from nitrogen, oxygen, and sulfur. In some embodiments, R^{3A} is an 8-10 membered bicyclic heteroaromatic ring having 1-5 heteroatoms independently selected from nitrogen, oxygen, and sulfur. In some embodiments, R^{3A} is a 7-12 membered saturated or partially unsaturated bicyclic or tricyclic heterocyclic ring having 1-4 heteroatoms independently selected from nitrogen, oxygen, and sulfur. In some embodiments, R^{3A} is a 5-12 membered saturated or partially unsaturated bicyclic carbocyclic ring that is optionally bridged or spirocyclic. In some embodiments, R^{3A} is a 5-12 membered saturated or partially unsaturated bicyclic heterocyclic ring that is optionally bridged or spirocyclic having 1-4 heteroatoms independently selected from nitrogen, oxygen, and sulfur.

In some embodiments, R^{3A} is substituted with 0, 1, 2, 3, or 4instances of R^{A}.

In some embodiments, R^{3A} is

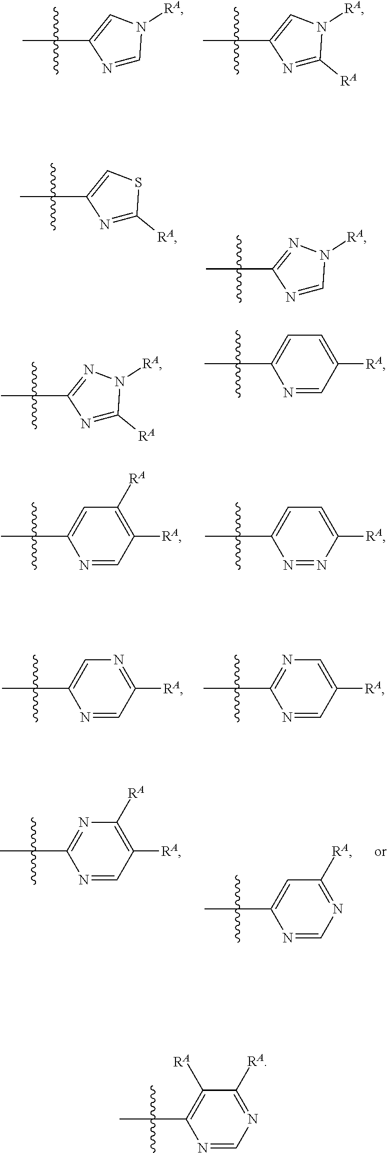

In some embodiments, R^{3A} and it's R^{A} substituents is

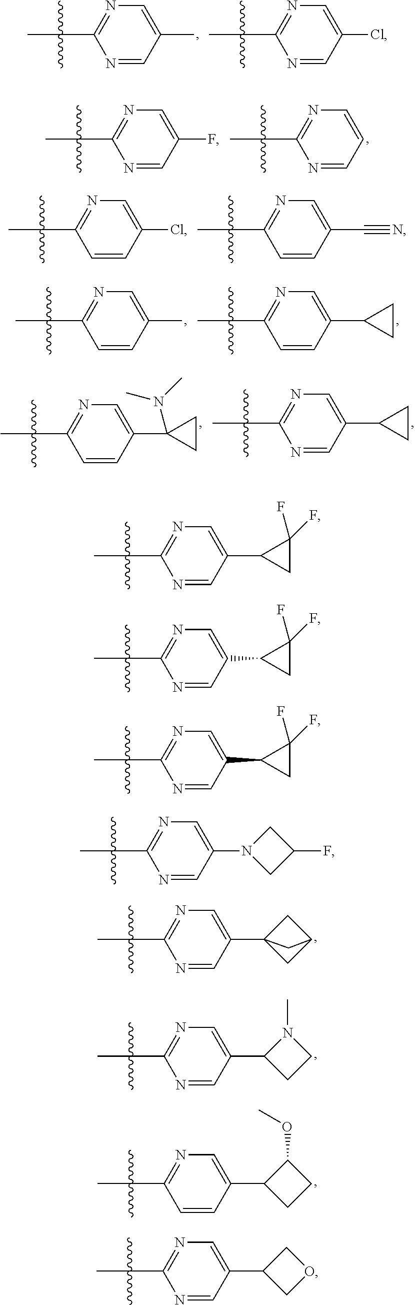

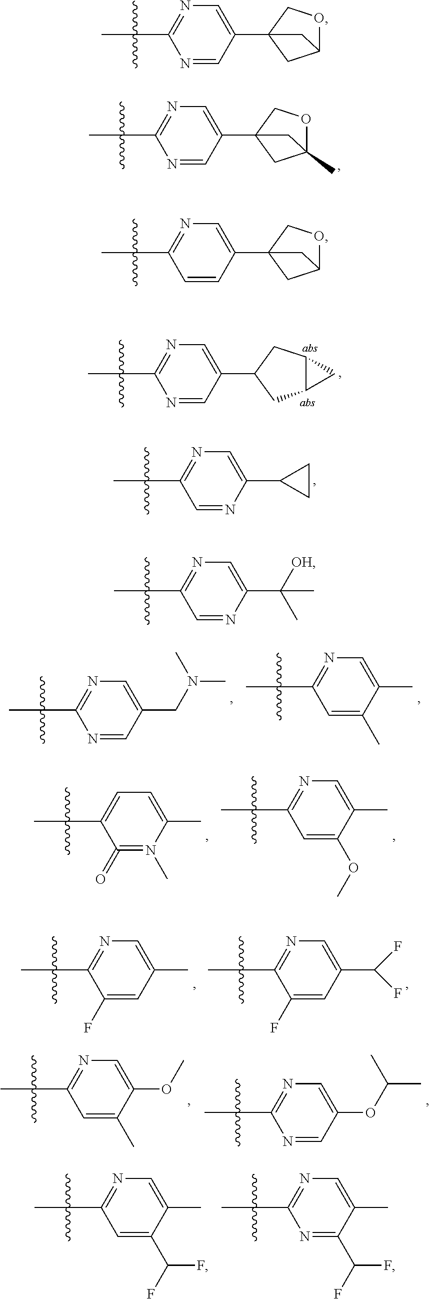

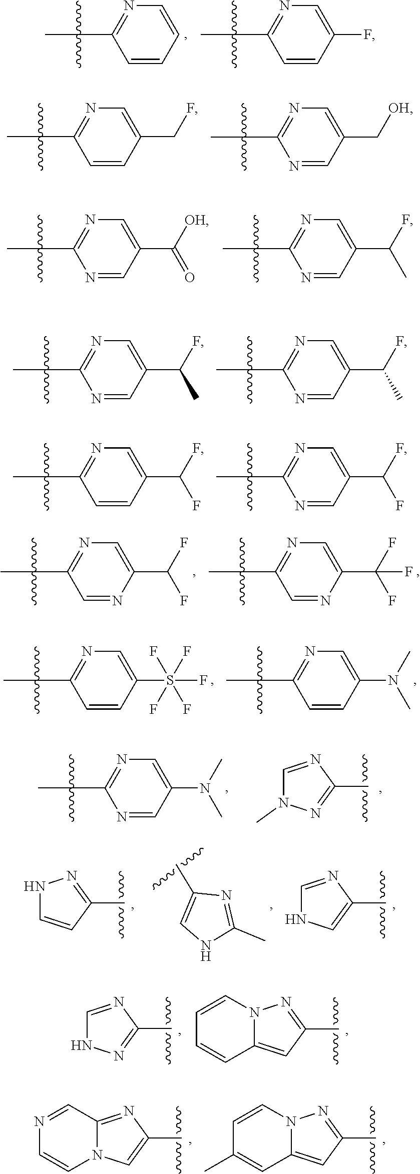

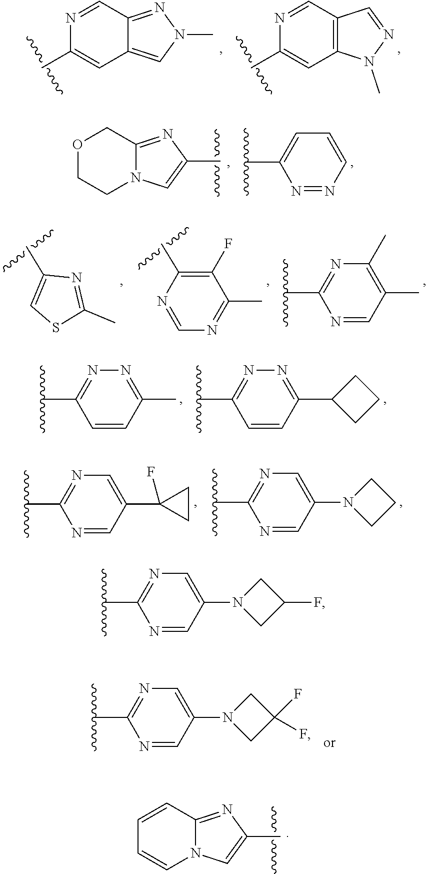

In some embodiments, R^{3A} and its R^{A} substituents have a structure of

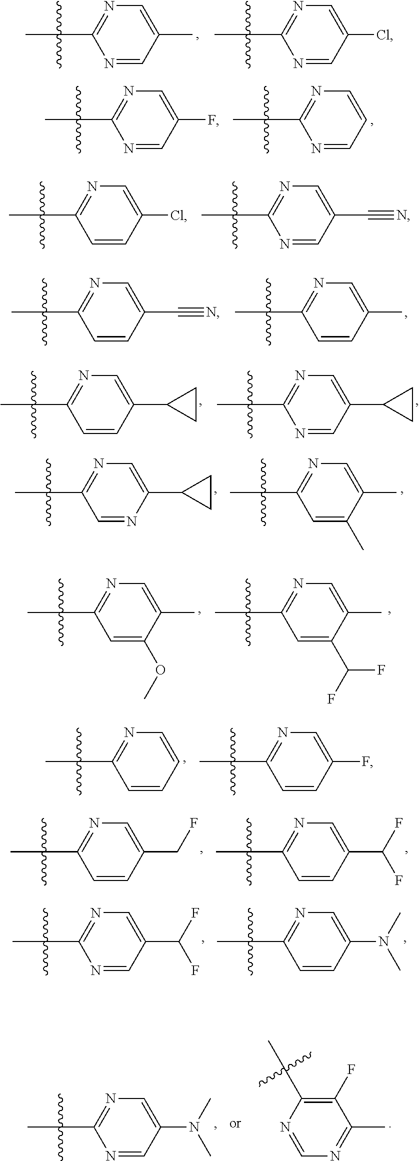

In some embodiments, R^{3A} is

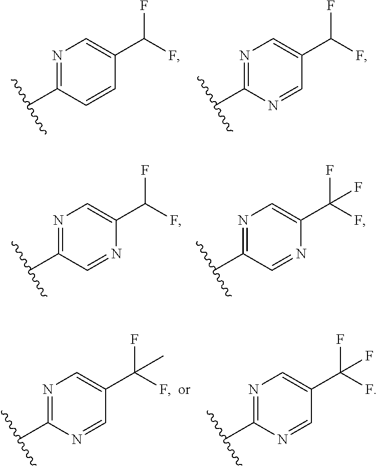

In some embodiments, R^{3A} is as depicted in the compounds of Table 1, below.

As defined generally above, Ring E is a cyclic group selected from phenyl; a 5-6 membered monocyclic heteroaromatic ring having 1-4 heteroatoms independently selected from nitrogen, oxygen, and sulfur; an 8-10 membered bicyclic heteroaromatic ring having 1-5 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-12 membered saturated or partially unsaturated bicyclic carbocyclic ring that is optionally bridged or spirocyclic; and a 5-12 membered saturated or partially unsaturated bicyclic heterocyclic ring that is optionally bridged or spirocyclic having 1-4 heteroatoms independently selected from nitrogen, oxygen, and sulfur; wherein Ring E is substituted with y instances of R^{A}.

In some embodiments, Ring E

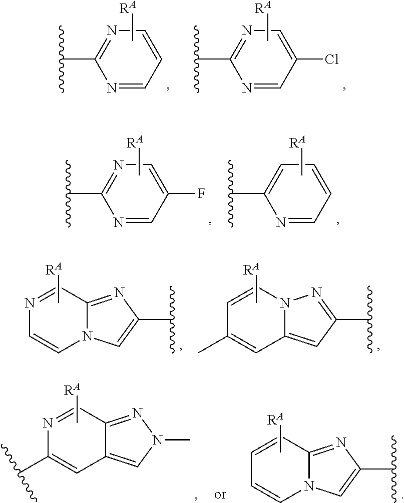

In some embodiments, Ring E is

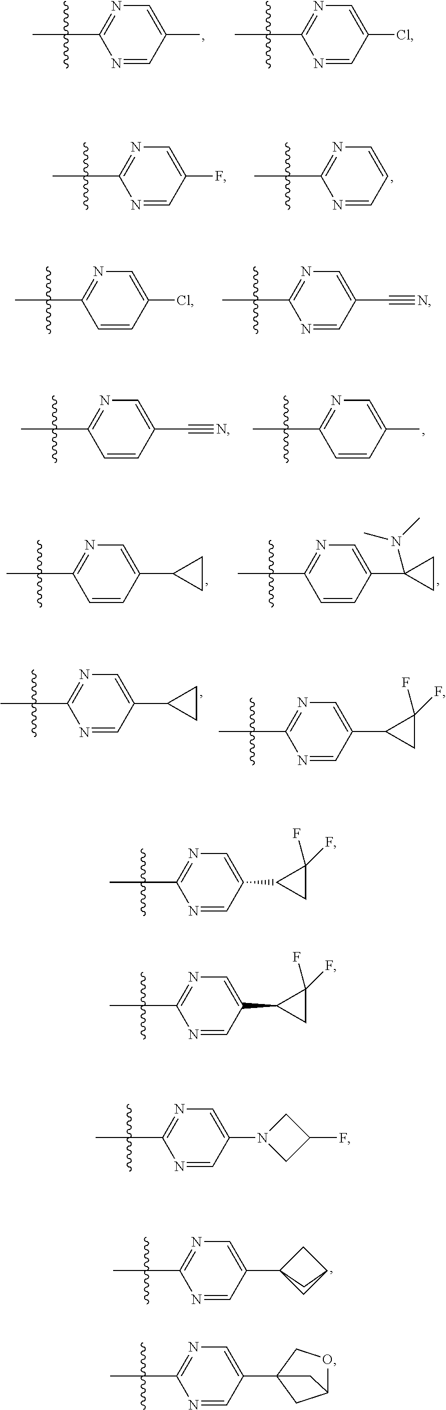

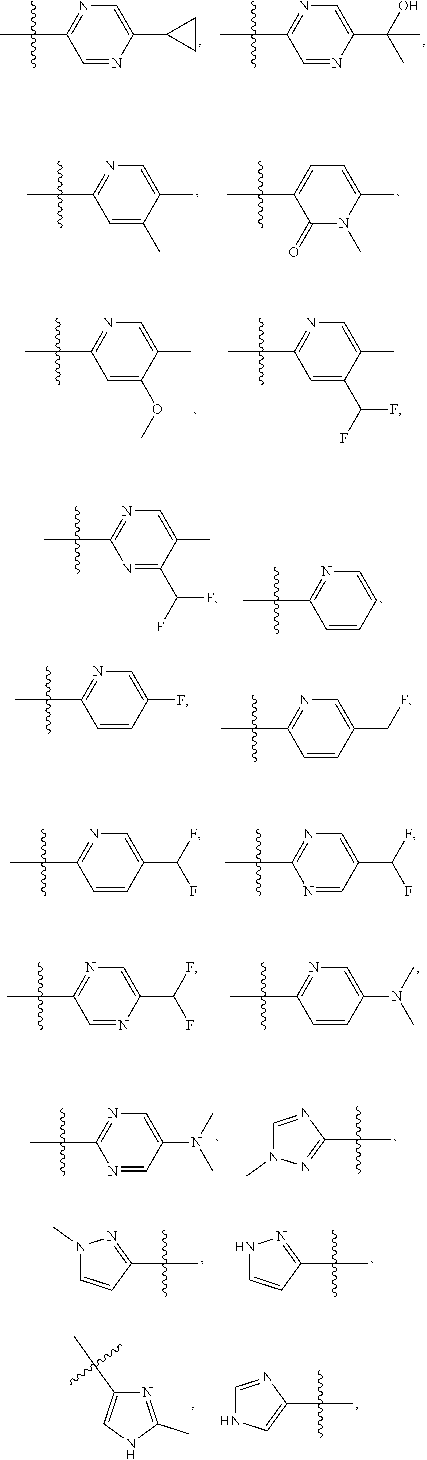

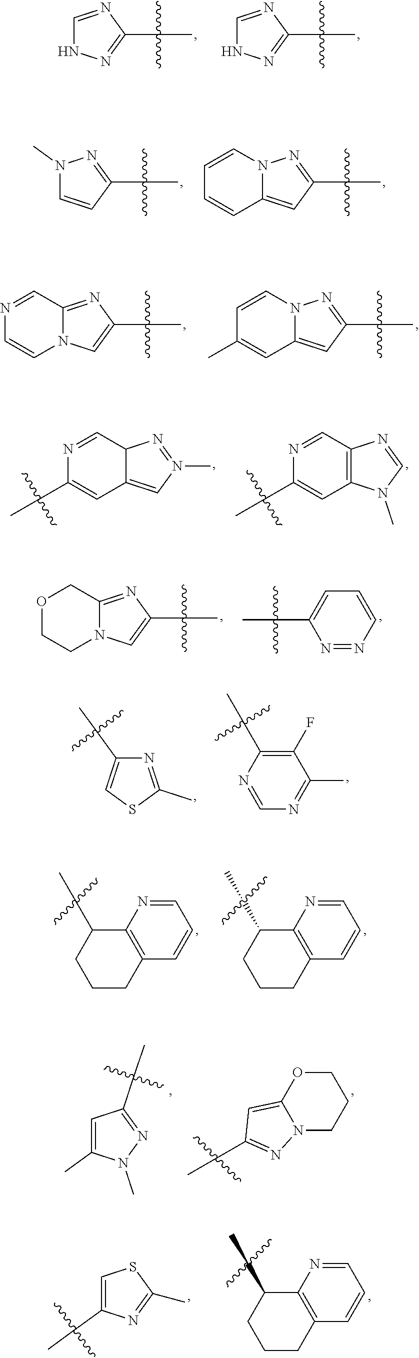

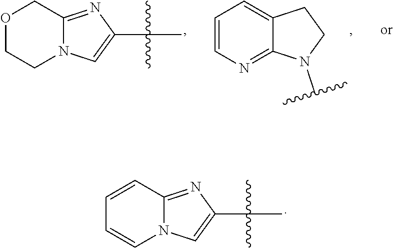 [SMILES: *c1cn2c(n1)COCC2 ; *N1CCc2cccnc21 ; *c1cn2ccccc2n1]

In some embodiments, Ring E is

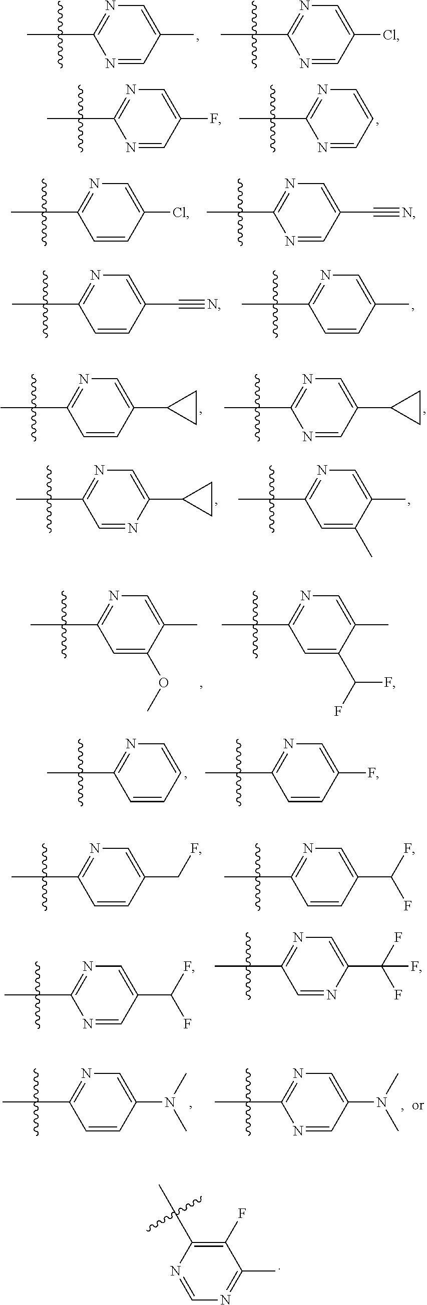

In some embodiments, Ring E is as depicted in the compounds of Table 1, below.

As defined generally above each R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N(R)OR; —OC(O)R; —OC(O)NR₂; —N(R)CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, a fused, bridged, or spiro 4-12 membered saturated or partially unsaturated carbocyclic ring and a fused, bridged, or spiro 4-12 membered saturated or partially unsaturated heterocyclic ring system (having 1-4 heteroatoms independently selected from nitrogen, oxygen, and sulfur), wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, and —NR₂; or two R^{A} on the same carbon atom together form oxo or a spiro cyclic group selected from a 3-8 membered saturated or partially unsaturated monocyclic carbocyclic ring and a 3-8 membered saturated or partially unsaturated monocyclic heterocyclic ring having 1-2 heteroatoms independently selected from nitrogen, oxygen, and sulfur; wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR, halogen, —CN, —NR₂, and —OR; halogen; —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or two R^{A} on adjacent atoms form a fused cyclic group selected from a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, and sulfur; phenyl; and a 5-6 membered heteroaryl ring having 1-4 heteroatoms independently selected from nitrogen, oxygen, and sulfur; wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, at least one R^{A} is

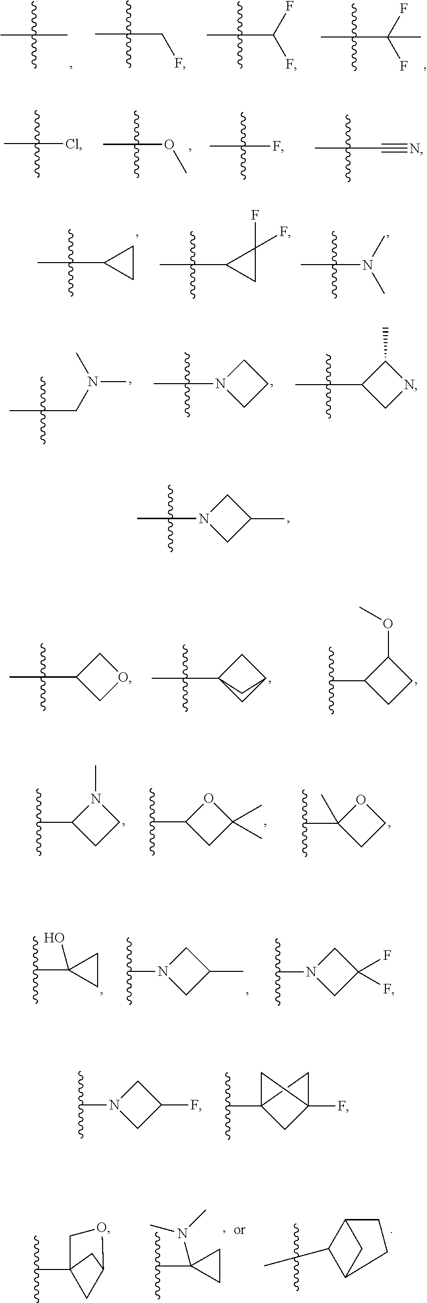

In some embodiments, at least one R^{A} is CHF₂, CF₃, or CF₂CH₃. In some embodiments, y is 1, and R^{A} is CHF₂, CF₃, or CF₂CH₃.

In some embodiments, R^{A} is as depicted in the compounds of Table 1, below.

As defined generally above, a is 1, 2, or 3, wherein when Z is a bond and Ring C is absent, R^{1A} is not hydrogen.

In some embodiments, a is 1. In some embodiments, a is 2. In some embodiments, a is 3.

In some embodiments, a is depicted in the compounds of Table 1, below.

As defined generally above, b is 0 or 1.

In some embodiments, b is 0. In some embodiments, b is 1.

In some embodiments, b is depicted in the compounds of Table 1, below.

As defined generally above, c is 0 or 1.

In some embodiments, c is 0. In some embodiments, c is 1.

In some embodiments, c is depicted in the compounds of Table 1, below.

As defined generally above, d is 0 or 1.

In some embodiments, d is 0. In some embodiments, d is 1.

In some embodiments, d is depicted in the compounds of Table 1, below.

As defined generally above, e is 0 or 1.

In some embodiments, e is 0. In some embodiments, e is 1.

In some embodiments, e is depicted in the compounds of Table 1, below.

In some embodiments, both d and e are 0.

As defined generally above, y is 0, 1, 2, 3, or 4.

In some embodiments, y is 0. In some embodiments, y is 1. In some embodiments, y is 2.

In some embodiments, y is 3. In some embodiments, y is 4.

In some embodiments, y is depicted in the compounds of Table 1, below.

As defined generally above, z is 0, 1, or 2.

In some embodiments, z is 0. In some embodiments, z is 1. In some embodiments, z is 2.

As defined generally above, R^{3B} is independently halogen, oxo, C₁₋₃ alkyl, C₂₋₆ alkenyl, C₂₋₃ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, or a 3-7 membered saturated or partially unsaturated monocyclic or bridged carbocyclic ring; a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents.

In some embodiments, R^{3B} is halogen. In some embodiments, R^{3B} is oxo. In some embodiments, R^{3B} is C₁₋₃ alkyl. In some embodiments, R^{3B} is C₂₋₆ alkenyl. In some embodiments, R^{3B} is C₂₋₃ alkynyl. In some embodiments, R^{3B} is C₁₋₃ alkoxy. In some embodiments, R^{3B} is C₁₋₃ haloalkyl. In some embodiments, R^{3B} is C₁₋₃ hydroxyalkyl. In some embodiments, R^{3B} is —OR. In some embodiments, R^{3B} is —SR. In some embodiments, R^{3B} is —NR₂. In some embodiments, R^{3B} is —SO₂R. In some embodiments, R^{3B} is —SO₂NR₂. In some embodiments, R^{3B} is —S(O)R. In some embodiments, R^{3B} is —S(O)NR₂. In some embodiments, R^{3B} is —C(O)R. In some embodiments, R^{3B} is —CO₂R. In some embodiments, R^{3B} is —C(O)NR₂. In some embodiments, R^{3B} is —C(O)N(R)OR. In some embodiments, R^{3B} is —OC(O)R. In some embodiments, R^{3B} is —OC(O)NR₂. In some embodiments, R^{3B} is —N(R)CO₂R. In some embodiments. In some embodiments, R^{3B} is —P(O)R₂. In some embodiments, R^{3B} is a 3-7 membered saturated or partially unsaturated monocyclic or bridged carbocyclic ring substituted with 0, 1, or 2 R⁸ substituents. In some embodiments, R^{3B} is a 3-7 membered saturated or partially unsaturated monocyclic heterocyclic ring having 1-2 heteroatoms independently selected from nitrogen, oxygen, and sulfur, substituted with 0, 1, or 2 R⁸ substituents. In some embodiments, R^{3B} is a 5-6 membered heteroaryl ring having 1-4 heteroatoms independently selected from nitrogen, oxygen, and sulfur, substituted with 0, 1, or 2 R⁸ substituents.

In some embodiments, R^{3B} is as depicted in the compounds of Table 1, below.

As defined generally above, each instance of R^{A} is independently selected from halogen; C₁₋₃ alkyl; C₂₋₆ alkenyl; C₂₋₃ alkynyl; C₁₋₃ alkoxy; C₁₋₃ haloalkyl; C₁₋₃ hydroxyalkyl; —CN; —OR; —SR; —SF₆; —C₀₋₃alkylene-NR²; —SO₂R; —SO₂NR₂; —S(O)R; —S(O)NR₂; —C(O)R; —CO₂R; —C(O)NR₂; —C(O)N(R)OR; —OC(O)R; —OC(O)NR₂; —N(R)CO₂R; or a 3-7 membered saturated or partially unsaturated monocyclic or bridged carbocyclic ring, substituted with 0, 1, 2, or 3 groups independently selected from halogen, C₁₋₃ alkyl, C₁₋₃ haloalkyl, —CN, —NR₂, and —OR; a 3-7 membered saturated or partially unsaturated monocyclic heterocyclic ring having 1-2 heteroatoms independently selected from nitrogen, oxygen, and sulfur, substituted with 0, 1, 2, or 3 groups independently selected from halogen, C₁₋₃ alkyl, C₁₋₃ haloalkyl, —CN, —NR₂, and —OR; or a 5-6 membered heteroaryl ring having 1-4 heteroatoms independently selected from nitrogen, oxygen, and sulfur, substituted with 0, 1, 2, or 3 groups independently selected from halogen, C₁₋₃ alkyl, C₁₋₃ haloalkyl, —CN, —NR₂, and —OR; a 4-12 membered saturated or partially unsaturated carbocyclic ring system that is fused, bridged, or spirocyclic, and a 4-12 membered saturated or partially unsaturated heterocyclic ring system that is fused, bridged, or spirocyclic (having 1-4 heteroatoms independently selected from nitrogen, oxygen, and sulfur), wherein said carbocyclic ring, heterocyclic ring, or heteroaryl ring is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or two R^{A} on the same carbon atom together form oxo or a spiro cyclic group selected from a 3-8 membered saturated or partially unsaturated monocyclic carbocyclic ring; and a 3-8 membered saturated or partially unsaturated monocyclic heterocyclic ring having 1-2 heteroatoms independently selected from nitrogen, oxygen, and sulfur; wherein the cyclic group formed by two R^{A} on the same carbon atom is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or two R^{A} on adjacent atoms form a fused cyclic group selected from a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, and sulfur; phenyl; and a 5-6 membered heteroaryl ring having 1-4 heteroatoms independently selected from nitrogen, oxygen, and sulfur; wherein the fused cyclic group formed by two R^{A} on adjacent atoms is substituted with 0, 1, 2, or 3 groups independently selected from a C₁₋₆ alkyl, C₁₋₆haloalkyl, C₁₋₆alkylene-CN, C₁₋₆alkylene-NR², C₁₋₆alkylene-OR —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, R^{A} is halogen. In some embodiments, R^{A} is C₁₋₃ alkyl. In some embodiments, R^{A} is C₂₋₆ alkenyl. In some embodiments, R^{A} is C₂₋₄ alkynyl. In some embodiments, R^{A} is C₁₋₃ alkoxy. In some embodiments, R^{A} is C₁₋₃ haloalkyl. In some embodiments, R^{A} is C₁₋₃ hydroxyalkyl. In some embodiments, R^{A} is —OR. In some embodiments, R^{A} is —SR. In some embodiments, R^{A} is —SF₆. In some embodiments, R^{A} is —NR₂. In some embodiments, R^{A} is —C₀₋₃alkylene-NR². In some embodiments, R^{A} is —SO₂R. In some embodiments, R^{A} is —SO₂NR₂. In some embodiments, R^{A} is —S(O)R. In some embodiments, R^{A} is —S(O)NR₂. In some embodiments, R^{A} is —C(O)R. In some embodiments, R^{A} is —CO₂R. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)CO₂R. In some embodiments, R^{A} is an optionally substituted C₁₋₆ aliphatic. In some embodiments, R^{A} is a 3-7 membered saturated or partially unsaturated monocyclic carbocyclic ring, optionally substituted with 1, 2, or 3 groups independently selected from halogen, C₁₋₃ alkyl, C₁₋₃ haloalkyl, —CN, —NR₂, and —OR. In some embodiments, R^{A} is a 3-8 membered saturated or partially unsaturated monocyclic heterocyclic ring having 1-2 heteroatoms independently selected from nitrogen, oxygen, and sulfur, optionally substituted with 1, 2, or 3 groups independently selected from halogen, C₁₋₃ alkyl, C₁₋₃ haloalkyl, —CN, —NR₂, and —OR. In some embodiments, R^{A} is a 5-6 membered heteroaryl ring having 1-4 heteroatoms independently selected from nitrogen, oxygen, and sulfur, optionally substituted with 1, 2, or 3 groups independently selected from halogen, C₁₋₃ alkyl, C₁₋₃ haloalkyl, —CN, —NR₂, and —OR. In some embodiments, R^{A} is a 4-12 membered saturated or partially unsaturated carbocyclic ring system that is fused, bridged, or spirocyclic or a 4-12 membered saturated or partially unsaturated heterocyclic ring system that is fused, bridged, or spirocyclic (having 1-4 heteroatoms independently selected from nitrogen, oxygen, and sulfur), wherein said carbocyclic or heterocyclic ring is substituted with 0-3 independently selected C₁₋₃ alkyl, C₁₋₃ alkoxy, C₁₋₃ haloalkyl, C₁₋₃ hydroxyalkyl, halogen, —CN, and —NR₂.

In some embodiments, R^{A} is a 3-8 membered saturated or partially unsaturated monocyclic heterocyclic ring having 1-2 heteroatoms independently selected from nitrogen, oxygen, and sulfur optionally substituted with 1, 2, or 3 groups independently selected from halogen, C₁₋₃ alkyl, C₁₋₃ haloalkyl, —CN, —NR₂, and —OR. In some embodiments, R^{A} is a 3-8 membered saturated or partially unsaturated monocyclic carbocyclic ring optionally substituted with 1, 2, or 3 groups independently selected from halogen, C₁₋₃ alkyl, C₁₋₃ haloalkyl, —CN, —NR₂, and —OR. In some embodiments, R^{A} is a 4-12 membered saturated or partially unsaturated carbocyclic ring system that is fused, bridged, or spirocyclic or a 4-12 membered saturated or partially unsaturated heterocyclic ring system that is fused, bridged, or spirocyclic (having 1-4 heteroatoms independently selected from nitrogen, oxygen, and sulfur), wherein said carbocyclic or heterocyclic ring is substituted with 0-3 independently selected C₁₋₃ alkyl, C₁₋₃ alkoxy, C₁₋₃ haloalkyl, C₁₋₃ hydroxyalkyl, halogen, —CN, and —NR₂. In some embodiments, R^{A} is halogen. In some embodiments, R^{A} is —CN. In some embodiments, R^{A} is —NO₂. In some embodiments, R^{A} is —OR. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)R. In some embodiments, R^{A} is —S(O)NR₂. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR; In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, two R^{A} on the same atom together form oxo.

In some embodiments, two R^{A} on the same atom together form a spiro cyclic group selected from 3-8 membered saturated or partially unsaturated monocyclic carbocyclic ring. In some embodiments, two R^{A} on the same atom together form a spiro cyclic group selected from a 3-8 membered saturated or partially unsaturated monocyclic heterocyclic ring having 1-2 heteroatoms independently selected from nitrogen, oxygen, and sulfur.

In some embodiments, the cyclic group formed by two R^{A} on the same atom is substituted with 1, 2, or 3 groups independently selected from a C₁₋₆alkyl, C₁₋₆haloalkyl, C₁₋₆alkylene-CN, C₁₋₆alkylene-NR₂, C₁₋₆alkylene-OR; halogen; —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, two R^{A} on adjacent atoms form a fused cyclic group selected from a 3-8 membered saturated or partially unsaturated monocyclic carbocyclic ring. In some embodiments, two R^{A} on adjacent atoms form a fused cyclic group selected from a 3-8 membered saturated or partially unsaturated monocyclic heterocyclic ring having 1-2 heteroatoms independently selected from nitrogen, oxygen, and sulfur, phenyl; and an optionally substituted 5-6 membered heteroaryl ring having 1-4 heteroatoms independently selected from nitrogen, oxygen, and sulfur.

In some embodiments, the fused cyclic group formed by two R^{A} on adjacent atoms is substituted with 1, 2, or 3 groups independently selected from C₁₋₆alkyl, C₁₋₆haloalkyl, C₁₋₆alkylene-CN, C₁₋₆alkylene-NR², C₁₋₆alkylene-OR; halogen; —CN; —NO²; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, Ring R^{A} is as depicted in the compounds of Table 1, below.

As defined generally above, each R^{1A} is independently halogen, —CN, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or R^{1A} is C₀₋₃alkylene-Cyc and Cyc is phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 4-12 membered saturated or partially unsaturated fused, bridged, or spiro carbocyclic ring system, and a fused, bridged or spiro 4-12 membered saturated or partially unsaturated heterocyclic ring system having 1-4 heteroatoms independently selected from nitrogen, oxygen, and sulfur, and Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —NR⁴₂; and a 5-6 membered heteroaryl ring having 1-4 heteroatoms independently selected from nitrogen, oxygen, and sulfur; or 1 R^{1A} and 1 R^{2A} combine to form a linker having a structure of —OC₂₋₆alkylene-NH— or —C₂₋₆alkylene-NH—, wherein the N forms a direct bond to the pyridine ring.

In some embodiments, R^{1A} is CH₂CH₃. In some embodiments, R^{1A} is CH₃. In some embodiments, R^{1A} is CF₃. In some embodiments, R^{1A} is CHF₂. In some embodiments, R^{1A} is CH₂CF₃. In some embodiments, R^{1A} is CH₂CHF₂.

In some embodiments, at least one R^{1A} is methyl,

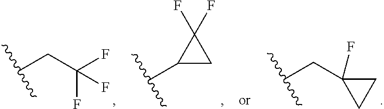

In some embodiments, Ring R^{1A} is as depicted in the compounds of Table 1, below.

In some embodiments, Ring C together with its R^{1A} substituents is as depicted in the compounds of Table 1, below.

In some embodiments, R^{1A} is halogen. In some embodiments, R^{1A} is —CN. In some embodiments, R^{1A} is oxo. In some embodiments, R^{1A} is —OR. In some embodiments, R^{1A} is —SR.

In some embodiments, R^{1A} is —NR₂. In some embodiments, R^{1A} is —SO₂R. In some embodiments, R^{1A} is —SO₂NR₂. In some embodiments, R^{1A} is —S(O)R. In some embodiments, R^{1A} is —S(O)NR₂. In some embodiments, R^{1A} is —C(O)R. In some embodiments, R^{1A} is —CO₂R. In some embodiments, R^{1A} is —C(O)NR₂. In some embodiments, R^{1A} is —C(O)N(R)OR. In some embodiments, R^{1A} is —OC(O)R. In some embodiments, R^{1A} is —OC(O)NR₂. In some embodiments, R^{1A} is —N(R)CO₂R. In some embodiments, R^{1A} is —N(R)C(O)R. In some embodiments, R^{1A} is —N(R)C(O)NR₂. In some embodiments, R^{1A} is —N(R)C(NR)R. In some embodiments, R^{1A} is —N(R)C(NR)NR₂. In some embodiments, R^{1A} is —N(R)NR₂. In some embodiments, R^{1A} is —N(R)SO₂NR₂. In some embodiments, R^{1A} is —N(R)SO₂R. In some embodiments, R^{1A} is —N═S(O)R₂. In some embodiments, R^{1A} is —S(NR)(O)R. In some embodiments, R^{1A} is —N(R)S(O)R. In some embodiments, R^{1A} is —N(R)CN. In some embodiments, R^{1A} is —P(O)(OR)₂. In some embodiments, R^{1A} is —P(O)R₂. In some embodiments, R^{1A} is C₀₋₃alkylene-Cyc and Cyc is phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 4-12 membered saturated or partially unsaturated fused, bridged, or spiro carbocyclic ring system, and a fused, bridged or spiro 4-12 membered saturated or partially unsaturated heterocyclic ring system having 1-4 heteroatoms independently selected from nitrogen, oxygen, and sulfur, and Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —N(R⁴)₂; and a 5-6 membered heteroaryl ring having 1-4 heteroatoms independently selected from nitrogen, oxygen, and sulfur. In some embodiments, R^{1A} is an optionally substituted phenyl. In some embodiments, R^{1A} is an optionally substituted 3-7 membered saturated or partially unsaturated monocyclic carbocyclic ring. In some embodiments, R^{1A} is an optionally substituted 3-7 membered saturated or partially unsaturated monocyclic heterocyclic ring having 1-2 heteroatoms independently selected from nitrogen, oxygen, and sulfur. In some embodiments, R^{1A} is a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), wherein said carbocyclyl or heterocyclyl is substituted with 0-3 independently selected C₁₋₃ alkyl, C₁₋₃ alkoxy, C₁₋₃ haloalkyl, C₁₋₃ hydroxyalkyl, halogen, —CN, and —NR₂. In some embodiments, R^{1A} is an optionally substituted 5-6 membered heteroaryl ring having 1-4 heteroatoms independently selected from nitrogen, oxygen, and sulfur.

In some embodiments, R^{1A} is halogen, —CN, oxo, —OR, —SO₂R, —C(O)NR₂, an optionally substituted C₁₋₆ alkyl, a 3-7 membered saturated monocyclic carbocyclic ring, or an optionally substituted 3-7 membered saturated monocyclic heterocyclic ring having 1-2 heteroatoms independently selected from nitrogen, oxygen, and sulfur.

In some embodiments, R^{1A} is halogen, —CN, —C(O)NR₂, an optionally substituted C₁₋₆ alkyl, a 3-7 membered saturated monocyclic carbocyclic ring, or an optionally substituted 3-7 membered saturated monocyclic heterocyclic ring having 1-2 heteroatoms independently selected from nitrogen, oxygen, and sulfur.

In some embodiments, R^{1A} is fluoro, chloro, —CN, oxo, —OMe, —O(CH₂)₂NMe₂, —O(CH₂)₂NEt₂, —O(CH₂)₂pyrrolidinyl, —O(CH₂)₂piperidinyl, —SO₂cyclopropyl, —SO₂Ph, —C(O)NH(CH₂)₂NMe₂, —C(O)NH(CH₂)₃NMe₂, methyl, ethyl, propyl, isopropyl, isobutyl, tert-butyl, isopentyl, cyclopentyl, cyclobutyl, methyloxycyclobutyl, —CHF₂, —CF₃, —CH₂CH₂F, —CH₂CHF₂, —CH₂CF₃, —CH₂NHMe, —CH₂NMeCO₂tBu, —CH₂SO₂Me, —CH₂CO₂H, —CH₂CO₂Me, —CH₂CONHMe, —CH₂CONMe₂, —CH₂CN, —CH(Me)CN, —CH(Me)₂CN, —CH₂cyclobutyl, —CH₂(methyloxycyclobutyl), —CH₂Oxetanyl, —CH₂(methyloxetanyl), —CH₂(fluorooxetanyl), —CH₂azetidinyl, —CH₂tetrahydrofuranyl, —CH₂pyrrolidinyl, —CH₂(N-methylpyrrolidinyl), —CH₂pyrazolyl, —CH₂tetrahydro-2H-pyranyl, —CH₂morpholinyl, —CH₂(N-methylmorpholinyl), —CH₂(N-methylpiperazinyl), —CH₂(N,N-dimethylpiperazinyl), —CH₂(N-acetylpiperazinyl), —CH₂pyridyl, benzyl, —CH₂(cyclopropylenyl)CN, —(CH₂)₂pyrrolidin-2-onyl, —(CH₂)₂CN, —(CH₂)₂NH₂, —(CH₂)₂oxetanyl, —(CH₂)₂azetidinyl, —(CH₂)₃NMe₂, piperazinyl, or N-methylpiperazinyl.

In some embodiments, R^{1A} is fluoro, chloro, —CN, —C(O)NH(CH₂)₂NMe₂, —C(O)NH(CH₂)₃NMe₂, methyl, ethyl, propyl, isopropyl, isobutyl, tert-butyl, isopentyl, cyclopentyl, cyclobutyl, —CHF₂, —CF₃, piperazinyl, or N-methylpiperazinyl.

In some embodiments, R^{1A} is

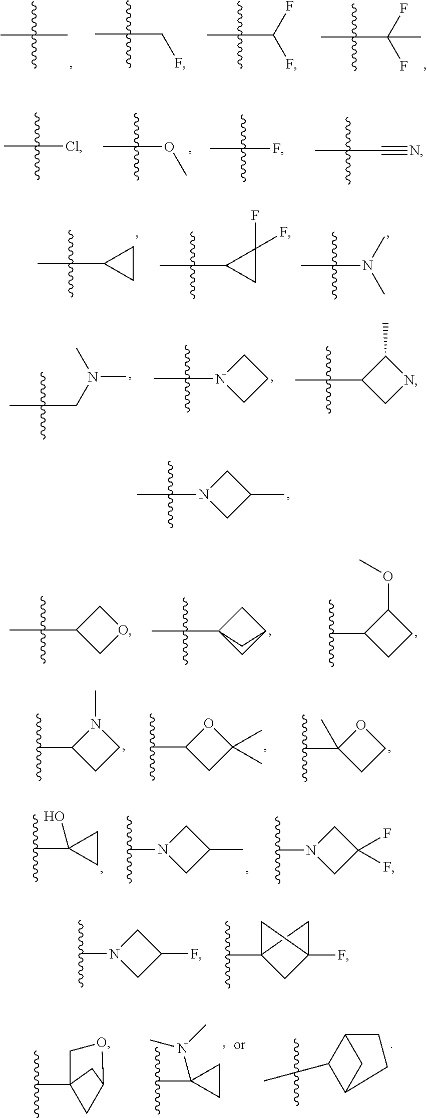

In some embodiments, R^{1A} are as depicted in the compounds of Table 1, below.

As defined generally above, each R is independently hydrogen, C₁₋₃ alkyl, C₂₋₆ alkenyl, C₂₋₃ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, a C₁₋₆ alkyl, cyclic group, and C₀₋₆ alkylene-cyclic group, wherein the cyclic group is selected from phenyl; a 3-7 membered saturated or partially unsaturated carbocyclic ring; a 3-7 membered saturated or partially unsaturated heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic ring having 0-2 heteroatoms independently selected from nitrogen, oxygen, and sulfur, wherein the cyclic group is substituted with 0, 1, 2, or 3 R^{S}; or two R groups on the same atom together with the atom on which they are attached form a cyclic group selected from a 4-7 membered saturated or partially unsaturated carbocycyl; a 4-7 membered saturated or partially unsaturated heterocyclyl having 1-3 heteroatoms independently selected from nitrogen, oxygen, and sulfur; and a 5-6 membered heteroaryl ring having 1-3 heteroatoms independently selected from nitrogen, oxygen, and sulfur, and the cyclic group is substituted with 0, 1, 2, or 3 R⁸.

In some embodiments, R is independently hydrogen. In some embodiments, R is independently C₁₋₃ alkyl. In some embodiments, R is independently C₂₋₆ alkenyl. In some embodiments, R is independently C₂₋₃ alkynyl. In some embodiments, R is independently C₁₋₃ alkoxy. In some embodiments, R is independently C₁₋₃ haloalkyl. In some embodiments, R is independently C₁₋₃ hydroxyalkyl. In some embodiments, R is independently —C(O)N(CH₃)₂. In some embodiments, R is independently —C(O)₂CH₃. In some embodiments, R is independently —C(O)₂C(CH₃)₃. In some embodiments, R is independently —C(O)₂CH(CH₃)₂. In some embodiments, R is independently —S(O)₂CH₃. In some embodiments, R is independently phenyl.

In some embodiments, R is a 3-7 membered saturated or partially unsaturated carbocyclic ring substituted with 0, 1, 2, or 3 R^{S}. In some embodiments, R is independently a 3-7 membered saturated or partially unsaturated heterocyclic ring having 1-2 heteroatoms independently selected from nitrogen, oxygen, and sulfur and substituted with 0, 1, 2, or 3 R^{S}. In some embodiments, R is independently a 5-6 membered heteroaryl ring having 1-4 heteroatoms independently selected from nitrogen, oxygen, and sulfur and substituted with 0, 1, 2, or 3 R^{S}. In some embodiments, R is independently a substituted 8-10 membered bicyclic heteroaryl ring having 1-4 heteroatoms independently selected from nitrogen, oxygen, and sulfur and substituted with 0, 1, 2, or 3 R^{S}. In some embodiments, R is independently a substituted 7-12 membered saturated or partially unsaturated bicyclic heterocyclic ring having 1-4 heteroatoms independently selected from nitrogen, oxygen, and sulfur and substituted with 0, 1, 2, or 3 R^{S}. In some embodiments, R is independently a substituted 5-8 membered saturated or partially unsaturated bridged bicyclic ring having 0-3 heteroatoms independently selected from nitrogen, oxygen, and sulfur and substituted with 0, 1, 2, or 3 R^{S}. In some embodiments, R is independently a substituted 6-10 membered saturated or partially unsaturated spirocyclic ring having 0-3 heteroatoms independently selected from nitrogen, oxygen, and sulfur and substituted with 0, 1, 2, or 3 R^{S}. In some embodiments, R is independently a substituted 6-11 membered saturated or partially unsaturated bicyclic ring having 0-2 heteroatoms independently selected from nitrogen, oxygen, and sulfur and substituted with 0, 1, 2, or 3 R^{S}. Each R^{S} is independently OH, C₁₋₃ alkyl, C₁₋₃ alkoxy, C₁₋₃ haloalkyl, C₁₋₃ hydroxyalkyl, —N(R⁴)₂, and halogen.

In some embodiments, two R groups on the same atom together with the atom on which they are attached form a substituted 4-7 membered saturated or partially unsaturated monocyclic carbocyclic ring substituted with 0, 1, 2, or 3 R^{S}. In some embodiments, two R groups on the same atom together with the atom on which they are attached form a substituted a 4-7 membered saturated or partially unsaturated monocyclic heterocyclic ring having 1-2 heteroatoms independently selected from nitrogen, oxygen, and sulfur and substituted with 0, 1, 2, or 3 R^{S}. In some embodiments, two R groups on the same atom together with the atom on which they are attached form a substituted 5-6 membered heteroaryl ring having 1-3 heteroatoms independently selected from nitrogen, oxygen, and sulfur and substituted with 0, 1, 2, or 3 R^{S}.

In some embodiments, R is as depicted in the compounds of Table 1, below.

In some embodiments, the compound of formula I is a compound of formula I-a-2 or I-a-2′ or I-a-2″:

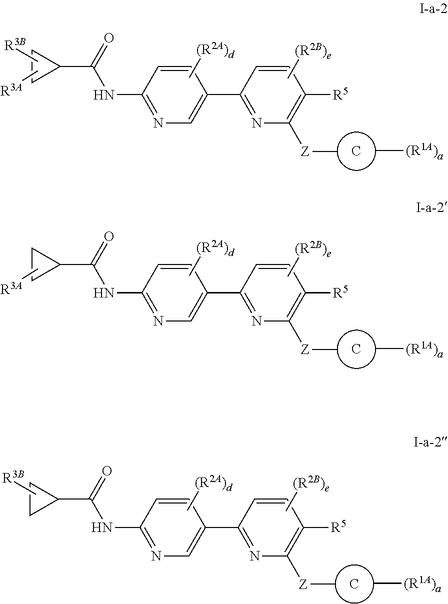

or a pharmaceutically acceptable salt thereof, wherein each of Z, R^{1A}, R^{3B}, R⁵, a, d, and e are as defined herein, both singly and in combination, and Ring C is a 5-membered heteroaryl having 1-3 heteroatoms independently selected from nitrogen, oxygen, and sulfur, R^{3A} is a 5-6 membered heteroaromatic ring having 1-4 heteroatoms independently selected from nitrogen, oxygen, and sulfur; and R^{2A} and R^{2B} are each independently selected from F, CH₃, and Cl.

In some embodiments, the compound of formula I is a compound of formula I-a-2, I-a-2′, or I-a-2″, wherein d and e are 0:

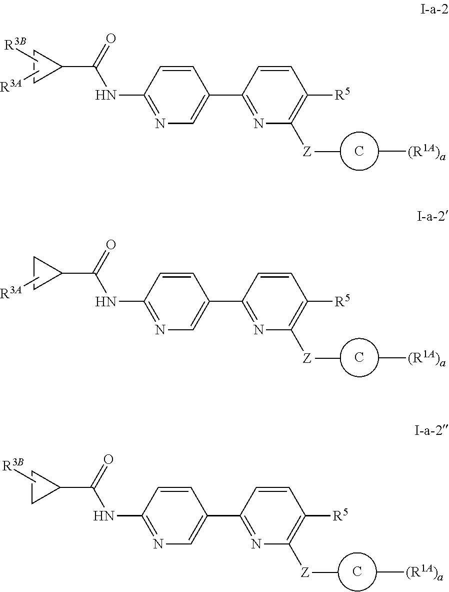

or a pharmaceutically acceptable salt thereof, wherein each of Z, R^{1A}, R^{3B} and a, are as defined herein, both singly and in combination, and Ring C is a 5-membered heteroaryl having 1-3 heteroatoms independently selected from nitrogen, oxygen, and sulfur, R^{3A} is a 5-6 membered heteroaromatic ring having 1-4 heteroatoms independently selected from nitrogen, oxygen, and sulfur; R⁵ is independently selected from —CN and C₁₋₄hydroxyalkyl.

In some embodiments, the compound of formula I is a compound of formula I-a-3, I-a-3′, or I-a-3″, wherein Z is a bond and d and e are 0:

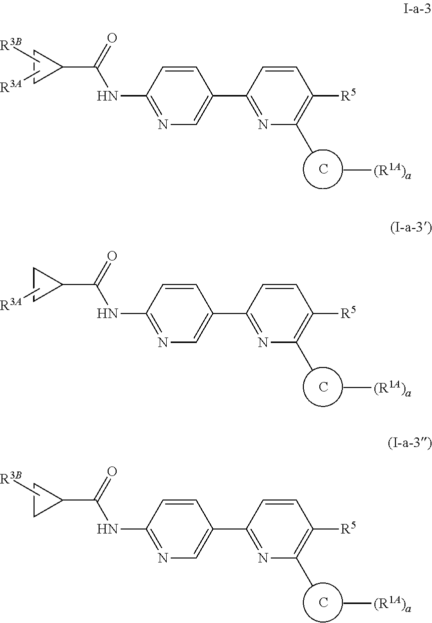

or a pharmaceutically acceptable salt thereof, wherein each of R^{1A}, R^{3B} and a, are as defined herein, both singly and in combination, and Ring C is a 5-membered heteroaryl having 1-3 heteroatoms independently selected from nitrogen, oxygen, and sulfur, R^{3A} is a 5-6 membered heteroaromatic ring having 1-4 heteroatoms independently selected from nitrogen, oxygen, and sulfur; R⁵ is independently selected from —CN and C₁₋₄hydroxyalkyl.

In some embodiments, the compound of formula I is a compound of formula I-a-2, I-a-2′, or I-a-2″ or a pharmaceutically acceptable salt thereof, wherein each of Z, R^{1A}, R^{3B}, R⁵, a, d, and e are as defined herein, both singly and in combination, and Ring C is a 5-10 membered saturated or partially unsaturated spirocyclic ring having 0-3 heteroatoms independently selected from nitrogen, oxygen, and sulfur, R^{3A} is a 5-6 membered heteroaromatic ring having 1-4 heteroatoms independently selected from nitrogen, oxygen, and sulfur; and R^{2A} and R^{2B} are each independently selected from F, CH₃, and Cl.

In some embodiments, the compound of formula I is a compound of formula I-a-2, I-a-2′, or I-a-2″ or a pharmaceutically acceptable salt thereof, wherein each of Z, R^{1A}, R^{3B} and a, are as defined herein, both singly and in combination, and Ring C is a substituted 5-10 membered saturated or partially unsaturated spirocyclic ring having 0-3 heteroatoms independently selected from nitrogen, oxygen, and sulfur, and sulfur, R^{3A} is a 5-6 membered heteroaromatic ring having 1-4 heteroatoms independently selected from nitrogen, oxygen, and sulfur; R⁵ is independently selected from —CN and C₁₋₄ hydroxyalkyl.

In some embodiments, Ring C of the compound of formula I-a-2, I-a-2′, or I-a-2″ is a 5-membered heteroaryl having 1-3 heteroatoms independently selected from nitrogen, oxygen, and sulfur.

In some embodiments, the compound of formula I is a compound of formula I-b′:

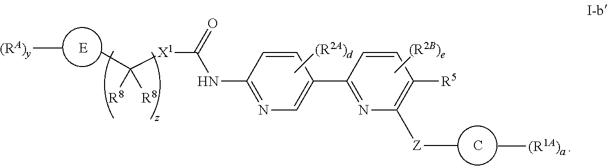

or a pharmaceutically acceptable salt thereof, wherein each of Z, R^{1A}, R^{A}, R⁵, R⁸, X¹, y, z, e, and d, are as defined herein, both singly and in combination, and Ring C is a 5-membered heteroaryl having 1-3 heteroatoms independently selected from nitrogen, oxygen, and sulfur, Ring E is a 5-6 membered heteroaromatic ring having 1-4 heteroatoms independently selected from nitrogen, oxygen, and sulfur; and R^{2A} and R^{2B} are each independently selected from F, CH₃, and Cl.

In some embodiments, the disclosure provides a compound of formula I-b′ or a pharmaceutically acceptable salt thereof, wherein each of Z, R^{1A}, R^{A}, R⁵, R⁸, X¹, y, z, e, and d, are as defined herein, both singly and in combination, and Ring C is a 5-membered heteroaryl having 1-3 heteroatoms independently selected from nitrogen, oxygen, and sulfur, Ring E is a is a 5-6 membered heteroaromatic ring having 1-4 heteroatoms independently selected from nitrogen, oxygen, and sulfur; R⁵ is independently selected from —CN and C₁₋₄hydroxyalkyl.

In some embodiments, the disclosure provides a compound of formula I-b′ or a pharmaceutically acceptable salt thereof, wherein each of Z, R^{1A}, R^{A}, R⁵, R⁸, X¹, y, z, e, and d, are as defined herein, both singly and in combination, and Ring C is a 5-10 membered saturated or partially unsaturated spirocyclic ring having 0-3 heteroatoms independently selected from nitrogen, oxygen, and sulfur, Ring E is a 5-6 membered heteroaromatic ring having 1-4 heteroatoms independently selected from nitrogen, oxygen, and sulfur.

In some embodiments, the disclosure provides a compound of formula I-b′ or a pharmaceutically acceptable salt thereof, wherein each of Z, R^{1A}, R^{A}, R⁵, R⁸, X¹, y, z, e, and d, are as defined herein, both singly and in combination, and Ring C is a substituted 5-10 membered saturated or partially unsaturated spirocyclic ring having 0-3 heteroatoms independently selected from nitrogen, oxygen, and sulfur, and sulfur, Ring E is a 5-6 membered heteroaromatic ring having 1-4 heteroatoms independently selected from nitrogen, oxygen, and sulfur; R⁵ is independently selected from —CN and C₁₋₄ hydroxyalkyl.

In some embodiments, the disclosure provides a compound of formula I-b′ or a pharmaceutically acceptable salt thereof, wherein each of Z, R^{1A}, R^{A}, R⁵, R⁸, X¹, y, z, e, and d, are as defined herein, both singly and in combination, and Ring C is a substituted 5-10 membered saturated or partially unsaturated spirocyclic ring having 0-3 heteroatoms independently selected from nitrogen, oxygen, and sulfur, and sulfur, Ring E is a 5-6 membered heteroaromatic ring having 1-4 heteroatoms independently selected from nitrogen, oxygen, and sulfur; R⁵ is independently selected from —CN and C₁₋₄ hydroxyalkyl.

In some embodiments, the compound of formula I is a compound of formula I-c-2:

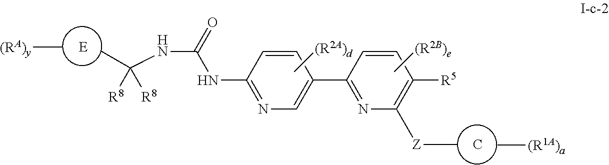

or a pharmaceutically acceptable salt thereof, wherein each of Z, R^{1A}, R^{A}, R⁵, R⁸, y, and d, are as defined herein, both singly and in combination, and Ring C is a 5-membered heteroaryl having 1-3 heteroatoms independently selected from nitrogen, oxygen, and sulfur, Ring E is a 5-6 membered heteroaromatic ring having 1-4 heteroatoms independently selected from nitrogen, oxygen, and sulfur; and R^{2A} and R^{2B} are each independently selected from F, CH₃, and Cl.

In some embodiments, the compound of formula I is a compound of formula I-c-2:

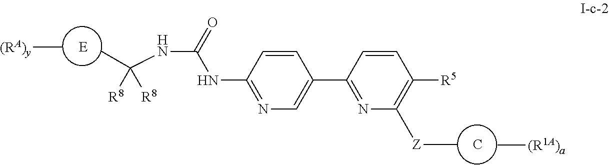

or a pharmaceutically acceptable salt thereof, wherein each of Z, R^{1A}, R^{A}, R⁸, y, and a, are as defined herein, both singly and in combination, and Ring C is a 5-membered heteroaryl having 1-3 heteroatoms independently selected from nitrogen, oxygen, and sulfur, Ring E is a is a 5-6 membered heteroaromatic ring having 1-4 heteroatoms independently selected from nitrogen, oxygen, and sulfur; R⁵ is independently selected from —CN and C₁₋₄hydroxyalkyl.

In some embodiments, the compound of formula I is a compound of formula I-c-2:

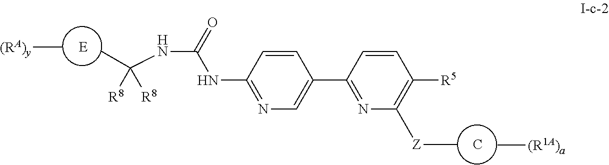

or a pharmaceutically acceptable salt thereof, wherein each of Z, R^{1A}, R^{A}, R⁵, R⁸, y, and a, are as defined herein, both singly and in combination, and Ring C is a 5-10 membered saturated or partially unsaturated spirocyclic ring having 0-3 heteroatoms independently selected from nitrogen, oxygen, and sulfur, Ring E is a 5-6 membered heteroaromatic ring having 1-4 heteroatoms independently selected from nitrogen, oxygen, and sulfur.

In some embodiments, the compound of formula I is a compound of formula I-c-2:

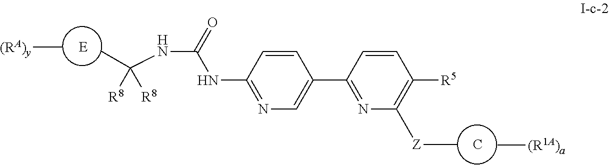

or a pharmaceutically acceptable salt thereof, wherein each of Z, R^{1A}, R^{A}, R⁸, y, and a, are as defined herein, both singly and in combination, and Ring C is a substituted 5-10 membered saturated or partially unsaturated spirocyclic ring having 0-3 heteroatoms independently selected from nitrogen, oxygen, and sulfur, and sulfur, Ring E is a 5-6 membered heteroaromatic ring having 1-4 heteroatoms independently selected from nitrogen, oxygen, and sulfur; R⁵ is independently selected from —CN and C₁₋₄ hydroxyalkyl.

Exemplary compounds of the disclosure are set forth in Table 1, or a pharmaceutically acceptable salt thereof. Table 1 identifies compounds by their IUPAC name and Table 2 lists the same compounds and shows their chemical structure. The chemical names in the present application are generated from the corresponding structures using either CHEMDRAW, or CHEMAXON. In some instances, chemical names generated from the structures may give a different structure when using the “Convert Name to Structure” function in CHEMDRAW. In the event of any discrepancy between Table 1's name for a compound and Table 2's structure for that same compound, Table 2's compound structures will dominate and identify the compound corresponding to each respective compound number (I-#) in Table 1.

TABLE 1

| I-# | IUPAC Name |
|---|---|
| I-1 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (5-methylpyridin-2-yl)cyclopropane-1-carboxamide |
| I-2 | (1S,2R)-2-(4,5-dimethylpyridin-2-yl)-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-3 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{2- |
| | methyl-2H-pyrazolo[3,4-c]pyridin-5-yl}cyclopropane-1- |
| | carboxamide |
| I-4 | (1S,2R)-2-[5-(fluoromethyl)pyridin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-5 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(4- |
| | methoxy-5-methylpyridin-2-yl)cyclopropane-1-carboxamide |
| I-6 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-7 | (1S,2R)-2-[5-(difluoromethyl)pyridin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-8 | (1S,2R)-N-{5-[(1S)-1-hydroxyethyl]-6-[5-methyl-1-(2,2,2- |
| | trifluoroethyl)-1H-1,2,3-triazol-4-yl]-[2,3′-bipyridin]-6′-yl}-2-(5- |
| | methylpyridin-2-yl)cyclopropane-1-carboxamide |
| I-9 | (1S,2R)-2-[5-(dimethylamino)pyridin-2-yl]-N-{5-[(1R*)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-10 | (1S,2R)-2-[5-(difluoromethyl)pyridin-2-yl]-N-{5-[(1S)-1- |
| | hydroxyethyl]-6-[5-methyl-1-(2,2,2-trifluoroethyl)-1H-1,2,3- |
| | triazol-4-yl]-[2,3′-bipyridin]-6′-yl}cyclopropane-1-carboxamide |
| I-11 | (1S,2R)-N-[6-(3-cyano-5-methyl-1H-pyrazol-1-yl)-5-[(1S)-1- |
| | hydroxyethyl]-[2,3′-bipyridin]-6′-yl]-2-[5- |
| | (difluoromethyl)pyridin-2-yl]cyclopropane-1-carboxamide |
| I-12 | (1S,2R)-N-{5-[(1R*)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methoxy-4-methylpyridin-2-yl)cyclopropane-1-carboxamide |
| I-13 | (1S,2R)-N-[6-(3-cyano-5-methyl-1H-pyrazol-1-yl)-5-[(1R)-1- |
| | hydroxyethyl]-[2,3′-bipyridin]-6′-yl]-2-(5-methylpyridin-2- |
| | yl)cyclopropane-1-carboxamide |
| I-14 | (1S,2R)-N-{6-[3-cyano-1-(2,2,2-trifluoroethyl)-1H-pyrazol-4- |
| | yl]-5-[(1S)-1-hydroxyethyl]-[2,3′-bipyridin]-6′-yl}-2-(5- |
| | methylpyridin-2-yl)cyclopropane-1-carboxamide |
| I-15 | (1S,2R)-N-{6-[1-(2,2-difluoroethyl)-3-methyl-1H-pyrazol-4-yl]- |
| | 5-[(1S)-1-hydroxyethyl]-[2,3′-bipyridin]-6′-yl}-2-(5- |
| | methylpyridin-2-yl)cyclopropane-1-carboxamide |
| I-16 | (1S,2R)-N-(6-{3-cyano-1H,4H,6H,7H-pyrano[4,3-c]pyrazol-1- |
| | yl}-5-[(1S)-1-hydroxyethyl]-[2,3′-bipyridin]-6′-yl)-2-(5- |
| | methylpyridin-2-yl)cyclopropane-1-carboxamide |
| I-17 | (1S,2R)-2-[4-(difluoromethyl)-5-methylpyridin-2-yl]-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide |
| I-18 | (1S,2R)-2-[5-(dimethylamino)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-19 | (1S,2R)-N-(6-{3-cyano-1H,4H,5H,7H-pyrano[3,4-c]pyrazol-1- |
| | yl}-5-[(1S)-1-hydroxyethyl]-[2,3′-bipyridin]-6′-yl)-2-(5- |
| | methylpyridin-2-yl)cyclopropane-1-carboxamide |
| I-20 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2- |
| | {5H,6H,8H-imidazo[2,1-c][1,4]oxazin-2-yl}cyclopropane-1- |
| | carboxamide |
| I-21 | (1S,2R)-N-(6-{3-cyano-1H,4H,5H,6H,7H-pyrazolo[4,3- |
| | c]pyridin-1-yl}-5-[(1R*)-1-hydroxyethyl]-[2,3′-bipyridin]-6′-yl)- |
| | 2-(5-methylpyridin-2-yl)cyclopropane-1-carboxamide |
| I-22 | (1S,2R)-N-{6-[1-(cyclopropylmethyl)-3-methyl-1H-pyrazol-4- |
| | yl]-5-[(1S)-1-hydroxyethyl]-[2,3′-bipyridin]-6′-yl}-2-(5- |
| | methylpyridin-2-yl)cyclopropane-1-carboxamide |
| I-23 | (1S,2R)-N-{6-[1-(2-fluoroethyl)-3-methyl-1H-pyrazol-4-yl]-5- |
| | [(1S)-1-hydroxyethyl]-[2,3′-bipyridin]-6′-yl}-2-(5-methylpyridin- |
| | 2-yl)cyclopropane-1-carboxamide |
| I-24 | (1S,2R)-N-[5-(1-hydroxyethyl)-6-[3-methyl-1-(2,2,2- |
| | trifluoroethyl)-1H-pyrazol-4-yl]-[2,3′-bipyridin]-6′-yl]-2-(5- |
| | methylpyridin-2-yl)cyclopropane-1-carboxamide |
| I-25 | (1S,2R)-N-(6-{3-cyano-1H,4H,5H,6H,7H-pyrazolo[4,3-c]pyridin- |
| | 1-yl}-5-(1-hydroxyethyl)-[2,3′-bipyridin]-6′-yl)-2-(5- |
| | methylpyridin-2-yl)cyclopropane-1-carboxamide |
| I-26 | (1S,2R)-N-{5-[(1R*)-2,2-difluoro-1-hydroxyethyl]-6-[3-methyl- |
| | 1-(2,2,2-trifluoroethyl)-1H-pyrazol-4-yl]-[2,3′-bipyridin]-6′-yl}- |
| | 2-(5-methylpyrimidin-2-yl)cyclopropane-1-carboxamide |
| I-27 | (1S,2R)-N-{5-[(1S)-1-hydroxyethyl]-6-[5-methyl-2-(2,2,2- |
| | trifluoroethyl)-2H-1,2,3-triazol-4-yl]-[2,3′-bipyridin]-6′-yl}-2-(5- |
| | methylpyridin-2-yl)cyclopropane-1-carboxamide |
| I-28 | (1S,2R)-2-(5-cyclopropylpyrimidin-2-yl)-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-29 | (1S,2R)-2-(4,5-dimethylpyrimidin-2-yl)-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-30 | (1S,2R)-N-[6-(3-cyano-5-methyl-1H-pyrazol-1-yl)-5-[(1R*)-1- |
| | hydroxyethyl]-[2,3′-bipyridin]-6′-yl]-2-(5-cyclopropylpyrimidin- |
| | 2-yl)cyclopropane-1-carboxamide |
| I-31 | (1S,2R)-N-{5-[(1S)-1-hydroxyethyl]-6-[5-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-32 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (5-methylpyridin-2-yl)cyclopropane-1-carboxamide |
| I-33 | (1S,2R)-2-(5-cyclopropylpyrazin-2-yl)-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-34 | (1S,2R)-N-[6-(1-cyclopropyl-3-methyl-1H-pyrazol-4-yl)-5-[(1S)- |
| | 1-hydroxyethyl]-[2,3′-bipyridin]-6′-yl]-2-(5-methylpyridin-2- |
| | yl)cyclopropane-1-carboxamide |
| I-35 | (1S,2R)-N-{5-[(1R*)-1-hydroxyethyl]-6-[3-methyl-1-(2,2,2- |
| | trifluoroethyl)-1H-pyrazol-4-yl]-[2,3′-bipyridin]-6′-yl}-2- |
| | {imidazo[1,2-a]pyrazin-2-yl}cyclopropane-1-carboxamide |
| I-36 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(6- |
| | methylpyridazin-3-yl)cyclopropane-1-carboxamide |
| I-37 | (1S,2R)-N-{6-[3-(difluoromethoxy)-5-methyl-1H-pyrazol-1-yl]- |
| | 5-[(1R*)-1-hydroxyethyl]-[2,3′-bipyridin]-6′-yl}-2-(5- |
| | methylpyridin-2-yl)cyclopropane-1-carboxamide |
| I-38 | (1S,2R)-2-(5-cyclopropylpyrimidin-2-yl)-N-{6-[1-(2,2- |
| | difluoroethyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-39 | (1S,2R)-2-(5-cyanopyridin-2-yl)-N-{5-[(1S)-1-hydroxyethyl]-6- |
| | [3-methyl-1-(2,2,2-trifluoroethyl)-1H-pyrazol-4-yl]-[2,3′- |
| | bipyridin]-6′-yl}cyclopropane-1-carboxamide |
| I-40 | 1-[5-(1-hydroxyethyl)-6′-[(1S,2R)-2-(5-methylpyridin-2- |
| | yl)cyclopropaneamido]-[2,3′-bipyridin]-6-yl]-1H,4H,5H,6H,7H- |
| | pyrazolo[4,3-c]pyridine-3-carboxamide |
| I-41 | (1S,2R)-N-(6-{3-cyano-5-methyl-1H,4H,5H,6H,7H- |
| | pyrazolo[4,3-c]pyridin-1-yl}-5-[(1R*)-1-hydroxyethyl]-[2,3′- |
| | bipyridin]-6′-yl)-2-(5-methylpyridin-2-yl)cyclopropane-1- |
| | carboxamide |
| I-42 | (1S,2R)-N-{6-[1-(3,3-difluoropropyl)-3-methyl-1H-pyrazol-4- |
| | yl]-5-[(1S)-1-hydroxyethyl]-[2,3′-bipyridin]-6′-yl}-2-(5- |
| | methylpyrimidin-2-yl)cyclopropane-1-carboxamide |
| I-43 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{1- |
| | methyl-1H-pyrazolo[4,3-c]pyridin-6-yl}cyclopropane-1- |
| | carboxamide |
| I-44 | (1S,2R)-N-{6-[1-(2,2-difluoroethyl)-5-methyl-1H-pyrazol-4-yl]- |
| | 5-[(1S)-1-hydroxyethyl]-[2,3′-bipyridin]-6′-yl}-2-(5- |
| | methylpyridin-2-yl)cyclopropane-1-carboxamide |
| I-45 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2- |
| | (pyridin-2-yl)cyclopropane-1-carboxamide |
| I-46 | (1S,2R)-N-[6-(3-cyano-5-methyl-1H-pyrazol-1-yl)-5-[(1R*)-1- |
| | hydroxyethyl]-[2,3′-bipyridin]-6′-yl]-2-(4,5-dimethylpyrimidin-2- |
| | yl)cyclopropane-1-carboxamide |
| I-47 | (1S,2R)-2-[5-(difluoromethyl)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-48 | (1S,2R)-N-{6-[1-(2,2-difluoroethyl)-3-methyl-1H-pyrazol-4-yl]- |
| | 5-[(1S)-1-hydroxyethyl]-[2,3′-bipyridin]-6′-yl}-2-(4,5- |
| | dimethylpyrimidin-2-yl)cyclopropane-1-carboxamide |
| I-49 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methylpyrimidin-2-yl)cyclopropane-1-carboxamide |
| I-50 | (1S,2R)-2-(5-fluoropyridin-2-yl)-N-{5-[(1S)-1-hydroxyethyl]-6- |
| | [3-methyl-1-(2,2,2-trifluoroethyl)-1H-pyrazol-4-yl]-[2,3′- |
| | bipyridin]-6′-yl}cyclopropane-1-carboxamide |
| I-51 | (1S,2R)-N-[6-(3-cyano-5-methyl-1H-pyrazol-1-yl)-5-[(1S)-1- |
| | hydroxyethyl]-[2,3′-bipyridin]-6′-yl]-2-[5- |
| | (difluoromethyl)pyrimidin-2-yl]cyclopropane-1-carboxamide |
| I-52 | (1S,2R)-N-{6-[1-(difluoromethyl)-4-methyl-1H-pyrazol-3-yl]-5- |
| | [(1R*)-1-hydroxyethyl]-[2,3′-bipyridin]-6′-yl}-2-(5- |
| | methylpyridin-2-yl)cyclopropane-1-carboxamide |
| I-53 | (1S,2R)-N-{5-[(1S)-1-hydroxyethyl]-6-{3-methyl-1-[(2R*)- |
| | 1,1,1-trifluoropropan-2-yl]-1H-pyrazol-4-yl}-[2,3′-bipyridin]-6′- |
| | yl}-2-(5-methylpyrimidin-2-yl)cyclopropane-1-carboxamide |
| I-54 | (1S,2R)-N-[6-(1-ethyl-3-methyl-1H-pyrazol-4-yl)-5-[(1R*)-1- |
| | hydroxyethyl]-[2,3′-bipyridin]-6′-yl]-2-(5-methylpyridin-2- |
| | yl)cyclopropane-1-carboxamide |
| I-55 | (1S,2R)-N-{6-[1-(2,2-difluoroethyl)-3-methyl-1H-pyrazol-4-yl]- |
| | 5-[(1S)-1-hydroxyethyl]-[2,3′-bipyridin]-6′-yl}-2-(5- |
| | methylpyrimidin-2-yl)cyclopropane-1-carboxamide |
| I-56 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methylpyrazin-2-yl)cyclopropane-1-carboxamide |
| I-57 | (1S,2R)-N-[6-(3-cyano-5-methyl-1H-pyrazol-1-yl)-5-[(1S)-1- |
| | hydroxyethyl]-[2,3′-bipyridin]-6′-yl]-2-(5-methylpyrimidin-2- |
| | yl)cyclopropane-1-carboxamide |
| I-58 | (1S,2R)-N-{6-[1-(2-fluoroethyl)-5-methyl-1H-pyrazol-4-yl]-5- |
| | [(1S)-1-hydroxyethyl]-[2,3′-bipyridin]-6′-yl}-2-(5-methylpyridin- |
| | 2-yl)cyclopropane-1-carboxamide |
| I-59 | (1S,2R)-N-{5-[(1S)-1-hydroxyethyl]-6-[3-methyl-1-(propan-2- |
| | yl)-1H-pyrazol-4-yl]-[2,3′-bipyridin]-6′-yl}-2-(5-methylpyridin- |
| | 2-yl)cyclopropane-1-carboxamide |
| I-60 | (1S,2R)-N-(6-{1,1-difluoro-5-azaspiro[2.4]heptan-5-yl}-5-[(1S)- |
| | 1-hydroxyethyl]-[2,3′-bipyridin]-6′-yl)-2-(5-methylpyridin-2- |
| | yl)cyclopropane-1-carboxamide |
| I-61 | (1S,2R)-N-{5-[(1S)-1-hydroxyethyl]-6-[4-methyl-1-(2,2,2- |
| | trifluoroethyl)-1H-pyrazol-3-yl]-[2,3′-bipyridin]-6′-yl}-2-(5- |
| | methylpyrimidin-2-yl)cyclopropane-1-carboxamide |
| I-62 | (1S,2R)-N-{6-[1-(cyclopropylmethyl)-5-methyl-1H-pyrazol-4- |
| | yl]-5-[(1S)-1-hydroxyethyl]-[2,3′-bipyridin]-6′-yl}-2-(5- |
| | methylpyridin-2-yl)cyclopropane-1-carboxamide |
| I-63 | (1S,2R)-N-{6-[3-(difluoromethyl)-5-methyl-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide |
| I-64 | (1S,2R)-N-{5-cyano-6-[(1-methylpiperidin-4-yl)oxy]-[2,3′- |
| | bipyridin]-6′-yl}-2-(5-methylpyridin-2-yl)cyclopropane-1- |
| | carboxamide |
| I-65 | (1S,2R)-N-(5-cyano-6-{[(4s)-1-methyl-1-azaspiro[3.3]heptan-6- |
| | yl]oxy}-[2,3′-bipyridin]-6′-yl)-2-(5-methylpyridin-2- |
| | yl)cyclopropane-1-carboxamide |
| I-66 | (1S,2R)-N-(5-cyano-6-{[(3R)-1-methylpiperidin-3-yl]oxy}-[2,3′- |
| | bipyridin]-6′-yl)-2-(5-methylpyridin-2-yl)cyclopropane-1- |
| | carboxamide |
| I-67 | (1S,2R)-N-(6-{3-cyano-1H,4H,5H,6H,7H-pyrazolo[4,3- |
| | c]pyridin-1-yl}-5-[(1R*)-1-hydroxyethyl]-[2,3′-bipyridin]-6′-yl)- |
| | 2-(5-methylpyridin-2-yl)cyclopropane-1-carboxamide |
| I-68 | (1S,2R)-N-(5-cyano-6-{[(2R*)-1,1-difluoropropan-2-yl]oxy}- |
| | [2,3′-bipyridin]-6′-yl)-2-(5-methylpyridin-2-yl)cyclopropane-1- |
| | carboxamide |
| I-69 | (1S,2R)-N-{6-[1-(difluoromethyl)-3-methyl-1H-pyrazol-4-yl]-5- |
| | [(1S)-1-hydroxyethyl]-[2,3′-bipyridin]-6′-yl}-2-(5-methylpyridin- |
| | 2-yl)cyclopropane-1-carboxamide |
| I-70 | (1S,2R)-N-{5-[(1S)-1-hydroxyethyl]-6-{[(3S)-1- |
| | methylpyrrolidin-3-yl]oxy}-[2,3′-bipyridin]-6′-yl}-2-(5- |
| | methylpyridin-2-yl)cyclopropane-1-carboxamide |
| I-71 | (1S,2R)-N-{5-cyano-6-[(1,1-difluoropropan-2-yl)oxy]-[2,3′- |
| | bipyridin]-6′-yl}-2-(5-methylpyridin-2-yl)cyclopropane-1- |
| | carboxamide |
| I-72 | (1S,2R)-N-{5-cyano-6-[(3s)-3-hydroxy-3-methylcyclobutoxy]- |
| | [2,3′-bipyridin]-6′-yl}-2-(5-methylpyridin-2-yl)cyclopropane-1- |
| | carboxamide |
| I-73 | (1S,2R)-N-{5-cyano-6-[(1s,3s)-3-(dimethylamino)cyclobutoxy]- |
| | [2,3′-bipyridin]-6′-yl}-2-(5-methylpyridin-2-yl)cyclopropane-1- |
| | carboxamide |
| I-74 | (1S,2R)-N-(5-cyano-6-{[(3S)-1-methylpyrrolidin-3-yl]oxy}- |
| | [2,3′-bipyridin]-6′-yl)-2-(5-methylpyridin-2-yl)cyclopropane-1- |
| | carboxamide |
| I-75 | (1S,2R)-N-{5-cyano-6-[(3r)-3-hydroxy-3-methylcyclobutoxy]- |
| | [2,3′-bipyridin]-6′-yl}-2-(5-methylpyridin-2-yl)cyclopropane-1- |
| | carboxamide |
| I-76 | (1S,2R)-N-{5-cyano-6-[(1-methylazetidin-3-yl)oxy]-[2,3′- |
| | bipyridin]-6′-yl}-2-(5-methylpyridin-2-yl)cyclopropane-1- |
| | carboxamide |
| I-77 | (1S,2R)-N-[5-cyano-6-({1-methyl-1-azaspiro[3.3]heptan-6- |
| | yl}oxy)-[2,3′-bipyridin]-6′-yl]-2-(5-methylpyridin-2- |
| | yl)cyclopropane-1-carboxamide |
| I-78 | (1S,2R)-N-{5-cyano-6-[(1r,3r)-3-(dimethylamino)cyclobutoxy]- |
| | [2,3′-bipyridin]-6′-yl}-2-(5-methylpyridin-2-yl)cyclopropane-1- |
| | carboxamide |
| I-79 | (1S,2R)-N-(5-cyano-6-{[(3R)-1-methylpyrrolidin-3-yl]oxy}- |
| | [2,3′-bipyridin]-6′-yl)-2-(5-methylpyridin-2-yl)cyclopropane-1- |
| | carboxamide |
| I-80 | (1S,2R)-N-(6-{[(2S)-1,1-difluoropropan-2-yl]oxy}-5-[(1S)-1- |
| | hydroxyethyl]-[2,3′-bipyridin]-6′-yl)-2-(5-methylpyridin-2- |
| | yl)cyclopropane-1-carboxamide |
| I-81 | (1S,2R)-N-{5-[(1S)-1-hydroxyethyl]-6-[5-methyl-1-(propan-2- |
| | yl)-1H-pyrazol-4-yl]-[2,3′-bipyridin]-6′-yl}-2-(5-methylpyridin- |
| | 2-yl)cyclopropane-1-carboxamide |
| I-82 | (1S,2R)-N-{5-cyano-6-[(1S,2S)-2-(dimethylamino)cyclobutoxy]- |
| | [2,3′-bipyridin]-6′-yl}-2-(5-methylpyridin-2-yl)cyclopropane-1- |
| | carboxamide |
| I-83 | (1S,2R)-N-{5-[(1S)-1-hydroxyethyl]-6-{3-methyl-1-[(2R*)- |
| | 1,1,1-trifluoropropan-2-yl]-1H-pyrazol-4-yl}-[2,3′-bipyridin]-6′- |
| | yl}-2-(5-methylpyrimidin-2-yl)cyclopropane-1-carboxamide |
| I-84 | (1S,2R)-N-{5-[(1S)-1-hydroxyethyl]-6-[5-methyl-3- |
| | (trifluoromethyl)-1H-pyrazol-1-yl]-[2,3′-bipyridin]-6′-yl}-2-(5- |
| | methylpyrimidin-2-yl)cyclopropane-1-carboxamide |
| I-85 | (1S,2R)-N-{6-[1-(difluoromethyl)-5-methyl-1H-pyrazol-4-yl]-5- |
| | [(1S)-1-hydroxyethyl]-[2,3′-bipyridin]-6′-yl}-2-(5-methylpyridin- |
| | 2-yl)cyclopropane-1-carboxamide |
| I-86 | (1S,2R)-N-[5-(difluoromethyl)-6-[3-methyl-1-(2,2,2- |
| | trifluoroethyl)-1H-pyrazol-4-yl]-[2,3′-bipyridin]-6′-yl]-2-(5- |
| | methylpyridin-2-yl)cyclopropane-1-carboxamide |
| I-87 | rel-N-{5-[(1R)-1-hydroxyethyl]-6-[3-methyl-1-(2,2,2- |
| | trifluoroethyl)-1H-pyrazol-4-yl]-[2,3′-bipyridin]-6′- |
| | yl}cyclopropanecarboxamide |
| I-88 | (1S,2R)-N-{5-[(1S)-1-hydroxyethyl]-6-(oxetan-3-yloxy)-[2,3′- |
| | bipyridin]-6′-yl}-2-(5-methylpyridin-2-yl)cyclopropane-1- |
| | carboxamide |
| I-89 | (1S,2R)-N-(5-cyano-6-{[(2R*)-1,1-difluoropropan-2-yl]oxy}- |
| | [2,3′-bipyridin]-6′-yl)-2-(5-methylpyridin-2-yl)cyclopropane-1- |
| | carboxamide |
| I-90 | (1S,2R)-N-[6-(1,3-dimethyl-1H-pyrazol-4-yl)-5-[(1R*)-1- |
| | hydroxyethyl]-[2,3′-bipyridin]-6′-yl]-2-(5-methylpyridin-2- |
| | yl)cyclopropane-1-carboxamide |
| I-91 | (1S,2R)-N-[5-(difluoromethyl)-6-{[(3S)-1-methylpyrrolidin-3- |
| | yl]oxy}-[2,3′-bipyridin]-6′-yl]-2-(5-methylpyridin-2- |
| | yl)cyclopropane-1-carboxamide |
| I-92 | (1S,2R)-N-[5-(hydroxymethyl)-6-[3-methyl-1-(2,2,2- |
| | trifluoroethyl)-1H-pyrazol-4-yl]-[2,3′-bipyridin]-6′-yl]-2-(5- |
| | methylpyridin-2-yl)cyclopropane-1-carboxamide |
| I-93 | (1S,2R)-N-{5-cyano-6-[3-methyl-1-(2,2,2-trifluoroethyl)-1H- |
| | pyrazol-4-yl]-[2,3′-bipyridin]-6′-yl}-2-(5-methylpyridin-2- |
| | yl)cyclopropane-1-carboxamide |
| I-94 | (1S,2R)-N-[6-(3-cyano-5-methyl-1H-pyrazol-1-yl)-5- |
| | (difluoromethyl)-[2,3′-bipyridin]-6′-yl]-2-(5-methylpyridin-2- |
| | yl)cyclopropane-1-carboxamide |
| I-95 | (1S,2R)-N-(6-{[(2R)-1,1-difluoropropan-2-yl]oxy}-5-[(1S)-1- |
| | hydroxyethyl]-[2,3′-bipyridin]-6′-yl)-2-(5-methylpyridin-2- |
| | yl)cyclopropane-1-carboxamide |
| I-96 | (1S,2R)-N-{5-cyano-6-[3-(difluoromethoxy)-5-methyl-1H- |
| | pyrazol-1-yl]-[2,3′-bipyridin]-6′-yl}-2-(5-methylpyridin-2- |
| | yl)cyclopropane-1-carboxamide |
| I-97 | (1S,2R)-N-{5-cyano-6-[3-(difluoromethyl)-5-methyl-1H- |
| | pyrazol-1-yl]-[2,3′-bipyridin]-6′-yl}-2-(5-methylpyridin-2- |
| | yl)cyclopropane-1-carboxamide |
| I-98 | (1S,2R)-N-{6-[3-(difluoromethyl)-5-methyl-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide |
| I-99 | (1S,2R)-N-{5-fluoro-6-[3-methyl-1-(2,2,2-trifluoroethyl)-1H- |
| | pyrazol-4-yl]-[2,3′-bipyridin]-6′-yl}-2-(5-methylpyridin-2- |
| | yl)cyclopropane-1-carboxamide |
| I-100 | (1S,2R)-N-{6-chloro-5-[(1S)-1-hydroxyethyl]-[2,3′-bipyridin]-6′- |
| | yl}-2-(5-methylpyridin-2-yl)cyclopropane-1-carboxamide |
| I-101 | (1S,2R)-N-{6-[3-(difluoromethoxy)-5-methyl-1H-pyrazol-1-yl]- |
| | 5-(difluoromethyl)-[2,3′-bipyridin]-6′-yl}-2-(5-methylpyridin-2- |
| | yl)cyclopropane-1-carboxamide |
| I-102 | (1S,2R)-N-[6-(3-cyano-5-methyl-1H-pyrazol-1-yl)-5-[(1S)-1- |
| | hydroxyethyl]-[2,3′-bipyridin]-6′-yl]-2-(5-methylpyridin-2- |
| | yl)cyclopropane-1-carboxamide |
| I-103 | (1S,2R)-N-{6-[1-(difluoromethyl)-4-methyl-1H-pyrazol-3-yl]-5- |
| | [(1R*)-1-hydroxyethyl]-[2,3′-bipyridin]-6′-yl}-2-(5- |
| | methylpyridin-2-yl)cyclopropane-1-carboxamide |
| I-104 | (1S,2R)-N-[6-(1-ethyl-3-methyl-1H-pyrazol-4-yl)-5-[(1R*)-1- |
| | hydroxyethyl]-[2,3′-bipyridin]-6′-yl]-2-(5-methylpyridin-2- |
| | yl)cyclopropane-1-carboxamide |
| I-105 | rel-N-{5-[(1R)-1-hydroxyethyl]-6-[3-methyl-1-(2,2,2- |
| | trifluoroethyl)-1H-pyrazol-4-yl]-[2,3′-bipyridin]-6′- |
| | yl}cyclopropanecarboxamide |
| I-106 | (1S,2R)-N-(6-{3-cyano-5-methyl-1H,4H,5H,6H,7H- |
| | pyrazolo[4,3-c]pyridin-1-yl}-5-[(1R*)-1-hydroxyethyl]-[2,3′- |
| | bipyridin]-6′-yl)-2-(5-methylpyridin-2-yl)cyclopropane-1- |
| | carboxamide |
| I-107 | (1S,2R)-N-{6-[3-(difluoromethoxy)-5-methyl-1H-pyrazol-1-yl]- |
| | 5-[(1R*)-1-hydroxyethyl]-[2,3′-bipyridin]-6′-yl}-2-(5- |
| | methylpyridin-2-yl)cyclopropane-1-carboxamide |
| I-108 | (1S,2R)-N-{6-[1-(2,2-difluoroethyl)-5-methyl-1H-pyrazol-4-yl]- |
| | 5-(2-hydroxypropan-2-yl)-[2,3′-bipyridin]-6′-yl}-2-(5- |
| | methylpyridin-2-yl)cyclopropane-1-carboxamide |
| I-109 | (1S,2R)-N-{5-[(1R*)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methoxy-4-methylpyridin-2-yl)cyclopropane-1-carboxamide |
| I-110 | (1S,2R)-2-[5-(dimethylamino)pyridin-2-yl]-N-{5-[(1R*)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-111 | (1S,2R)-N-{5-[(1R*)-1-hydroxyethyl]-6-[3-methyl-1-(2,2,2- |
| | trifluoroethyl)-1H-pyrazol-4-yl]-[2,3′-bipyridin]-6′-yl}-2- |
| | {imidazo[1,2-a]pyrazin-2-yl}cyclopropane-1-carboxamide |
| I-112 | (1S,2R)-N-{5-[(1S)-1-hydroxyethyl]-6-(prop-1-yn-1-yl)-[2,3′- |
| | bipyridin]-6′-yl}-2-(5-methylpyridin-2-yl)cyclopropane-1- |
| | carboxamide |
| I-113 | (1S,2R)-N-{5-[(1R*)-1-hydroxyethyl]-6-[3-methyl-1-(2,2,2- |
| | trifluoroethyl)-1H-pyrazol-4-yl]-[2,3′-bipyridin]-6′-yl}-2- |
| | {imidazo[1,2-a]pyridin-2-yl}cyclopropane-1-carboxamide |
| I-114 | (1S,2R)-N-{5-[(1R*)-1-methoxyethyl]-6-[3-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-115 | (1S,2R)-N-[6-(1,3-dimethyl-1H-pyrazol-4-yl)-5-[(1R*)-1- |
| | methoxyethyl]-[2,3′-bipyridin]-6′-yl]-2-(5-methylpyridin-2- |
| | yl)cyclopropane-1-carboxamide |
| I-116 | (1S,2R)-N-[6-(1,3-dimethyl-1H-pyrazol-4-yl)-5-[(1R*)-1- |
| | methoxyethyl]-[2,3′-bipyridin]-6′-yl]-2-(5-methylpyridin-2- |
| | yl)cyclopropane-1-carboxamide |
| I-117 | (1S,2R)-N-{5-[(1R*)-1-methoxyethyl]-6-[3-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-118 | (1S,2R)-N-{6-[(1,1-difluoropropan-2-yl)oxy]-5-(1- |
| | hydroxyethyl)-[2,3′-bipyridin]-6′-yl}-2-(5-methylpyridin-2- |
| | yl)cyclopropane-1-carboxamide |
| I-119 | (1S,2R)-N-{5-[(1S)-1-aminoethyl]-6-[3-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-120 | (1S,2R)-N-{6-[1-(2,2-difluoroethyl)-3-methyl-1H-pyrazol-4-yl]- |
| | 5-(methoxymethyl)-[2,3′-bipyridin]-6′-yl}-2-(5-methylpyridin-2- |
| | yl)cyclopropane-1-carboxamide |
| I-121 | (1S,2R)-N-[6-(3-cyano-5-methyl-1H-pyrazol-1-yl)-5-[(1R*)-1- |
| | hydroxyethyl]-[2,3′-bipyridin]-6′-yl]-2-(5-cyclopropylpyrimidin- |
| | 2-yl)cyclopropane-1-carboxamide |
| I-122 | (1S,2R)-N-[6-(3-cyano-5-methyl-1H-pyrazol-1-yl)-5-[(1R*)-1- |
| | hydroxyethyl]-[2,3′-bipyridin]-6′-yl]-2-(4,5-dimethylpyrimidin-2- |
| | yl)cyclopropane-1-carboxamide |
| I-123 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{1- |
| | methyl-1H-pyrazolo[3,4-c]pyridin-5-yl}cyclopropane-1- |
| | carboxamide |
| I-124 | (1S,2R)-N-(5-cyano-6-{[(3S)-1-methylpiperidin-3-yl]oxy}-[2,3′- |
| | bipyridin]-6′-yl)-2-(5-methylpyridin-2-yl)cyclopropane-1- |
| | carboxamide |
| I-125 | (1S,2R)-N-{5-[(1R*)-2,2-difluoro-1-hydroxyethyl]-6-[3-methyl- |
| | 1-(2,2,2-trifluoroethyl)-1H-pyrazol-4-yl]-[2,3′-bipyridin]-6′-yl}- |
| | 2-(5-methylpyrimidin-2-yl)cyclopropane-1-carboxamide |
| I-126 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{2- |
| | methyl-2H-pyrazolo[4,3-c]pyridin-6-yl}cyclopropane-1- |
| | carboxamide |
| I-127 | (1S,2R)-N-{5-[(1R)-1-hydroxyethyl]-6-[3-methyl-1-(2,2,2- |
| | trifluoroethyl)-1H-pyrazol-4-yl]-[2,3′-bipyridin]-6′-yl}-2-(4- |
| | methoxy-5-methylpyridin-2-yl)cyclopropane-1-carboxamide |
| I-128 | methyl 6-[3-methyl-1-(2,2,2-trifluoroethyl)-1H-pyrazol-4-yl]-6′- |
| | [(1S,2R)-2-(5-methylpyridin-2-yl)cyclopropaneamido]-[2,3′- |
| | bipyridine]-5-carboxylate |
| I-129 | (1S,2R)-N-{5-[(1S)-1-hydroxyethyl]-6-[(3S)-oxolan-3-yloxy]- |
| | [2,3′-bipyridin]-6′-yl}-2-(5-methylpyridin-2-yl)cyclopropane-1- |
| | carboxamide |
| I-130 | (1S,2R)-N-{5-[(1S)-1-hydroxyethyl]-6-methoxy-[2,3′-bipyridin]- |
| | 6′-yl}-2-(5-methylpyridin-2-yl)cyclopropane-1-carboxamide |
| I-131 | (1S,2R)-N-{6-[(1R,5S)-3-azabicyclo[3.1.0]hexan-3-yl]-5-[(1R*)- |
| | 1-hydroxyethyl]-[2,3′-bipyridin]-6′-yl}-2-(5-methylpyridin-2- |
| | yl)cyclopropane-1-carboxamide |
| I-132 | (1S,2R)-N-{5-[(1S)-1-hydroxyethyl]-6-[(3R)-oxolan-3-yloxy]- |
| | [2,3′-bipyridin]-6′-yl}-2-(5-methylpyridin-2-yl)cyclopropane-1- |
| | carboxamide |
| I-133 | (1S,2R)-N-(6-{5-azaspiro[2.4]heptan-5-yl}-5-[(1R*)-1- |
| | hydroxyethyl]-[2,3′-bipyridin]-6′-yl)-2-(5-methylpyridin-2- |
| | yl)cyclopropane-1-carboxamide |
| I-134 | (1S,2R)-N-{5-cyano-6-[(1-methylpiperidin-4-yl)methoxy]-[2,3′- |
| | bipyridin]-6′-yl}-2-(5-methylpyridin-2-yl)cyclopropane-1- |
| | carboxamide |
| I-135 | (1S,2R)-N-{5-[(1R)-1-hydroxyethyl]-6-[3-methyl-1-(2,2,2- |
| | trifluoroethyl)-1H-pyrazol-4-yl]-[2,3′-bipyridin]-6′-yl}-2-(5- |
| | methylpyridin-2-yl)cyclopropane-1-carboxamide |
| I-136 | (1S,2R)-N-{5-cyano-6-methoxy-[2,3′-bipyridin]-6′-yl}-2-(5- |
| | methylpyridin-2-yl)cyclopropane-1-carboxamide |
| I-137 | (1R,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{1- |
| | methyl-1H-pyrazolo[4,3-c]pyridin-6-yl}cyclopropane-1- |
| | carboxamide |
| I-138 | (1R,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(4- |
| | methoxy-5-methylpyridin-2-yl)cyclopropane-1-carboxamide |
| I-139 | (1S,2R)-N-{6-[1-(2,2-difluoroethyl)-3-methyl-1H-pyrazol-4-yl]- |
| | 5-(2-hydroxypropan-2-yl)-[2,3′-bipyridin]-6′-yl}-2-(5- |
| | methylpyridin-2-yl)cyclopropane-1-carboxamide |
| I-140 | (1S,2R)-N-{5-[(1S)-1-hydroxyethyl]-6-[(oxetan-3-yl)methyl]- |
| | [2,3′-bipyridin]-6′-yl}-2-(5-methylpyridin-2-yl)cyclopropane-1- |
| | carboxamide |
| I-141 | (1S,2R)-N-[6-(1,3-dimethyl-1H-pyrazol-4-yl)-5-[(1R*)-1- |
| | hydroxyethyl]-[2,3′-bipyridin]-6′-yl]-2-(5-methylpyridin-2- |
| | yl)cyclopropane-1-carboxamide |
| I-142 | (1S,2R)-N-{5-cyano-6-[(oxetan-3-yl)methyl]-[2,3′-bipyridin]-6′- |
| | yl}-2-(5-methylpyridin-2-yl)cyclopropane-1-carboxamide |
| I-143 | (1S,2R)-N-{5-[(1R)-1-hydroxyethyl]-6-[3-methyl-1-(propan-2- |
| | yl)-1H-pyrazol-4-yl]-[2,3′-bipyridin]-6′-yl}-2-(5-methylpyridin- |
| | 2-yl)cyclopropane-1-carboxamide |
| I-144 | (1S,2R)-N-[5-acetyl-6-(1,3-dimethyl-1H-pyrazol-4-yl)-[2,3′- |
| | bipyridin]-6′-yl]-2-(5-methylpyridin-2-yl)cyclopropane-1- |
| | carboxamide |
| I-145 | (1S,2R)-N-{6-[(1R,5S)-3-azabicyclo[3.1.0]hexan-3-yl]-5-[(1R*)- |
| | 1-hydroxyethyl]-[2,3′-bipyridin]-6′-yl}-2-(5-methylpyridin-2- |
| | yl)cyclopropane-1-carboxamide |
| I-146 | (1S,2R)-N-{6-[1-(2-fluoroethyl)-3-methyl-1H-pyrazol-4-yl]-5- |
| | [(1R)-1-hydroxyethyl]-[2,3′-bipyridin]-6′-yl}-2-(5-methylpyridin- |
| | 2-yl)cyclopropane-1-carboxamide |
| I-147 | (1S,2R)-N-(6-{5-azaspiro[2.4]heptan-5-yl}-5-[(1R*)-1- |
| | hydroxyethyl]-[2,3′-bipyridin]-6′-yl)-2-(5-methylpyridin-2- |
| | yl)cyclopropane-1-carboxamide |
| I-148 | (1S,2R)-N-(5-cyano-3-methyl-6-{[(3S)-1-methylpyrrolidin-3- |
| | yl]oxy}-[2,3′-bipyridin]-6′-yl)-2-(5-methylpyridin-2- |
| | yl)cyclopropane-1-carboxamide |
| I-149 | (1R,2R)-N-{5-[(1R)-1-hydroxyethyl]-6-[3-methyl-1-(2,2,2- |
| | trifluoroethyl)-1H-pyrazol-4-yl]-[2,3′-bipyridin]-6′-yl}-2-(4- |
| | methoxy-5-methylpyridin-2-yl)cyclopropane-1-carboxamide |
| I-150 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| I-151 | (1S,2R)-N-(6-{1-[(1S*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| I-152 | (1S,2R)-2-[5-(1-bicyclo[1.1.1]pentanyl)pyrimidin-2-yl]-N-[5-[5- |
| | [(1S)-1-hydroxyethyl]-6-[3-methyl-1-(2,2,2- |
| | trifluoroethyl)pyrazol-4-yl]-2-pyridyl]-2- |
| | pyridyl]cyclopropanecarboxamide |
| I-153 | (1S,2R)-N-(6-(3-(difluoromethyl)-1-(2,2,2-trifluoroethyl)-1H- |
| | pyrazol-4-yl)-5-((S)-1-hydroxyethyl)-[2,3′-bipyridin]-6′-yl)-2-(5- |
| | (difluoromethyl)pyrimidin-2-yl)cyclopropane-1-carboxamide |
| I-154 | N-[5-[5-[(1S)-1-hydroxyethyl]-6-[3-methyl-1-(2,2,2- |
| | trifluoroethyl)pyrazol-4-yl]-2-pyridyl]-2-pyridyl]-3-(5-methyl-2- |
| | pyridyl) propanamide |
| I-155 | (1S,2R)-2-(5-((1S,4s)-2-oxabicyclo[2.1.1]hexan-4-yl)pyrimidin- |
| | 2-yl)-N-(5-((S)-1-hydroxyethyl)-6-(3-methyl-1-(2,2,2- |
| | trifluoroethyl)-1H-pyrazol-4-yl)-[2,3′-bipyridin]-6′- |
| | yl)cyclopropane-1-carboxamide |
| I-156 | (1S,2R)-2-(5-(1-(dimethylamino)cyclopropyl)pyridin-2-yl)-N-(5- |
| | ((S)-1-hydroxyethyl)-6-(3-methyl-1-(2,2,2-trifluoroethyl)-1H- |
| | pyrazol-4-yl)-[2,3′-bipyridin]-6′-yl)cyclopropane-1-carboxamide |
| I-157 | (1S,2R)-2-(5-(difluoromethyl)pyrimidin-2-yl)-N-(5-((R*)-1- |
| | fluoroethyl)-6-(3-methyl-1-(2,2,2-trifluoroethyl)-1H-pyrazol-4- |
| | yl)-[2,3′-bipyridin]-6′-yl)cyclopropane-1-carboxamide (Isomer 1) |
| I-158 | (1S,2R)-2-(5-(difluoromethyl)pyrimidin-2-yl)-N-(5-((R*)-1- |
| | fluoroethyl)-6-(3-methyl-1-(2,2,2-trifluoroethyl)-1H-pyrazol-4- |
| | yl)-[2,3′-bipyridin]-6′-yl)cyclopropane-1-carboxamide (Isomer 2) |
| I-159 | (1S,2R)-2-(5-(difluoromethyl)pyrimidin-2-yl)-N-(5-((S)-1- |
| | hydroxyethyl)-6-(5-methyl-1-(2,2,2-trifluoroethyl)-1H-1,2,3- |
| | triazol-4-yl)-[2,3′-bipyridin]-6′-yl)cyclopropane-1-carboxamide |
| I-160 | (1S,2R)-2-(5-(difluoromethyl)pyrimidin-2-yl)-N-(5-((S)-1- |
| | hydroxyethyl)-6-(5-methyl-2-(2,2,2-trifluoroethyl)-2H-1,2,3- |
| | triazol-4-yl)-[2,3′-bipyridin]-6′-yl)cyclopropane-1-carboxamide |
| I-161 | (1S,2R)-N-(5-(1,1-difluoroethyl)-6-(3-methyl-1-(2,2,2- |
| | trifluoroethyl)-1H-pyrazol-4-yl)-[2,3′-bipyridin]-6′-yl)-2-(5- |
| | (difluoromethyl)pyrimidin-2-yl)cyclopropane-1-carboxamide |
| I-162 | (1S,2R)-2-(5-((R*)-2,2-difluorocyclopropyl)pyrimidin-2-yl)-N- |
| | (5-((S)-1-hydroxyethyl)-6-(3-methyl-1-(2,2,2-trifluoroethyl)-1H- |
| | pyrazol-4-yl)-[2,3′-bipyridin]-6′-yl)cyclopropane-1-carboxamide |
| | (Isomer 1) |
| I-163 | (1S,2R)-2-(5-((R*)-2,2-difluorocyclopropyl)pyrimidin-2-yl)-N- |
| | (5-((S)-1-hydroxyethyl)-6-(3-methyl-1-(2,2,2-trifluoroethyl)-1H- |
| | pyrazol-4-yl)-[2,3′-bipyridin]-6′-yl)cyclopropane-1-carboxamide |
| | (Isomer 2) |
| I-164 | (1S,2R)-N-(6-(1-((R*)-2,2-difluorocyclopropyl)-3-methyl-1H- |
| | pyrazol-4-yl)-5-((S)-1-hydroxyethyl)-[2,3′-bipyridin]-6′-yl)-2-(5- |
| | (3-fluoroazetidin-1-yl)pyridin-2-yl)cyclopropane-1-carboxamide |
| I-165 | (1S,2R)-N-[5-[5-[(1S)-1-hydroxyethyl]-6-[3-methyl-1-(2,2,2- |
| | trifluoroethyl)pyrazol-4-yl]-2-pyridyl]-2-pyridyl]-2-[5-(1- |
| | hydroxy-1-methyl-ethyl)pyrazin-2-yl]cyclopropanecarboxamide |
| I-166 | (1S,2R)-2-(4-(difluoromethyl)-5-methylpyrimidin-2-yl)-N-(5- |
| | ((S)-1-hydroxyethyl)-6-(3-methyl-1-(2,2,2-trifluoroethyl)-1H- |
| | pyrazol-4-yl)-[2,3′-bipyridin]-6′-yl)cyclopropane-1-carboxamide |
| I-167 | 1-(5-((S)-1-hydroxyethyl)-6-(3-methyl-1-(2,2,2-trifluoroethyl)- |
| | 1H-pyrazol-4-yl)-[2,3′-bipyridin]-6′-yl)-3-((R*)-1-(5- |
| | methylpyridin-2-yl)ethyl)urea (Isomer 1) |
| I-168 | 1-(5-((S)-1-hydroxyethyl)-6-(3-methyl-1-(2,2,2-trifluoroethyl)- |
| | 1H-pyrazol-4-yl)-[2,3′-bipyridin]-6′-yl)-3-((S*)-1-(5- |
| | methylpyridin-2-yl)ethyl)urea (Isomer 2) |
| I-169 | (1S,2R)-N-(5-((S)-1-hydroxyethyl)-6-(1-(2,2,2-trifluoroethyl)- |
| | 1,4,6,7-tetrahydropyrano[4,3-c]pyrazol-3-yl)-[2,3′-bipyridin]-6′- |
| | yl)-2-(5-methylpyrimidin-2-yl)cyclopropane-1-carboxamide |
| I-170 | (1S,2R)-2-(1,6-dimethyl-2-oxo-1,2-dihydropyridin-3-yl)-N-(5- |
| | ((S)-1-hydroxyethyl)-6-(3-methyl-1-(2,2,2-trifluoroethyl)-1H- |
| | pyrazol-4-yl)-[2,3′-bipyridin]-6′-yl) cyclopropane-1-carboxamide |
| I-171 | (1S,2R)-2-(1,6-dimethyl-2-oxo-1,2-dihydropyridin-3-yl)-N-(5- |
| | ((S)-1-hydroxyethyl)-6-(3-methyl-1-(2,2,2-trifluoroethyl)-1H- |
| | pyrazol-4-yl)-[2,3′-bipyridin]-6′-yl) cyclopropane-1-carboxamide |
| I-172 | N-(5-((S)-1-hydroxyethyl)-6-(3-methyl-1-(2,2,2-trifluoroethyl)- |
| | 1H-pyrazol-4-yl)-[2,3′-bipyridin]-6′-yl)-2-((R*)-5,6,7,8- |
| | tetrahydroquinolin-8-yl)acetamide (Isomer 1) |
| I-173 | N-(5-((S)-1-hydroxyethyl)-6-(3-methyl-1-(2,2,2-trifluoroethyl)- |
| | 1H-pyrazol-4-yl)-[2,3′-bipyridin]-6′-yl)-2-((R*)-5,6,7,8- |
| | tetrahydroquinolin-8-yl)acetamide (Isomer 2) |
| I-174 | (1S,2R)-2-(5-(difluoromethyl)pyridin-2-yl)-N-(5-((2R*,4R**)-4- |
| | hydroxytetrahydrofuran-2-yl)-6-(3-methyl-1-(2,2,2- |
| | trifluoroethyl)-1H-pyrazol-4-yl)-[2,3′-bipyridin]-6′- |
| | yl)cyclopropane-1-carboxamide (Isomer 1) |
| I-175 | (1S,2R)-2-(5-(difluoromethyl)pyridin-2-yl)-N-(5-((2R*,4S**)-4- |
| | hydroxytetrahydrofuran-2-yl)-6-(3-methyl-1-(2,2,2- |
| | trifluoroethyl)-1H-pyrazol-4-yl)-[2,3′-bipyridin]-6′- |
| | yl)cyclopropane-1-carboxamide (Isomer 2) |
| I-176 | (1S,2R)-2-(5-(difluoromethyl)pyridin-2-yl)-N-(5-((2R*,4S**)-4- |
| | hydroxytetrahydrofuran-2-yl)-6-(3-methyl-1-(2,2,2- |
| | trifluoroethyl)-1H-pyrazol-4-yl)-[2,3′-bipyridin]-6′- |
| | yl)cyclopropane-1-carboxamide (Isomer 3) |
| I-177 | (1S,2R)-2-(5-(difluoromethyl)pyridin-2-yl)-N-(5-((2R*,4R**)-4- |
| | hydroxytetrahydrofuran-2-yl)-6-(3-methyl-1-(2,2,2- |
| | trifluoroethyl)-1H-pyrazol-4-yl)-[2,3′-bipyridin]-6′- |
| | yl)cyclopropane-1-carboxamide (Isomer 4) |
| I-178 | (1S,2R)-N-(5′-fluoro-5-((S)-1-hydroxyethyl)-6-(3-methyl-1- |
| | (2,2,2-trifluoroethyl)-1H-pyrazol-4-yl)-[2,3′-bipyridin]-6′-yl)-2- |
| | (5-methylpyrimidin-2-yl)cyclopropane-1-carboxamide |
| I-179 | (1S,2R)-N-(5-((R*)-1-hydroxy-2-methylpropyl)-6-(3-methyl-1- |
| | (2,2,2-trifluoroethyl)-1H-pyrazol-4-yl)-[2,3′-bipyridin]-6′-yl)-2- |
| | (5-methylpyrimidin-2-yl)cyclopropane-1-carboxamide (Isomer 1) |
| I-180 | (1S,2R)-N-(5-((R*)-1-hydroxy-2-methylpropyl)-6-(3-methyl-1- |
| | (2,2,2-trifluoroethyl)-1H-pyrazol-4-yl)-[2,3′-bipyridin]-6′-yl)-2- |
| | (5-methylpyrimidin-2-yl)cyclopropane-1-carboxamide (Isomer 2) |
| I-181 | N-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-2-{1H,2H,3H- |
| | pyrrolo[2,3-b]pyridin-1-yl}acetamide |
| I-182 | (1S,2R)-2-[5-(difluoromethyl)pyrimidin-2-yl]-N-{6-[3-(1- |
| | fluorocyclopropyl)-5-methyl-1H-pyrazol-1-yl]-5-[(1R*)-1- |
| | hydroxyethyl]-[2,3′-bipyridin]-6′-yl}cyclopropane-1- |
| | carboxamide (Isomer 1) |
| I-183 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(trifluoromethyl)pyridin-2-yl]cyclopropane-1-carboxamide |
| | (Isomer 2) |
| I-184 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 1) |
| I-185 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 2) |
| I-186 | (1S,2R)-N-{6-[1-(2,2-difluorocyclopropyl)-4-methyl-1H- |
| | pyrazol-3-yl]-5-[(1S)-1-hydroxyethyl]-[2,3′-bipyridin]-6′-yl}-2- |
| | (5-methylpyrimidin-2-yl)cyclopropane-1-carboxamide |
| I-187 | (1S,2R)-2-[5-(difluoromethyl)pyridin-2-yl]-N-{5-[(1S)-1- |
| | hydroxyethyl]-6-{[(1R*,4R**,5S*)-2-methyl-2- |
| | azabicyclo[3.1.0]hexan-4-yl]oxy}-[2,3′-bipyridin]-6′- |
| | yl}cyclopropane-1-carboxamide (Isomer 1) |
| I-188 | (1S,2R)-2-[5-(difluoromethyl)pyridin-2-yl]-N-{5-[(1S)-1- |
| | hydroxyethyl]-6-{[(1R*,4R**,5S*)-2-methyl-2- |
| | azabicyclo[3.1.0]hexan-4-yl]oxy}-[2,3′-bipyridin]-6′- |
| | yl}cyclopropane-1-carboxamide (Isomer 2) |
| I-189 | (1S,2R)-2-[5-(difluoromethyl)pyridin-2-yl]-N-{5-[(1S)-1- |
| | hydroxyethyl]-6-{[(1R*,4R**,5S*)-2-methyl-2- |
| | azabicyclo[3.1.0]hexan-4-yl]oxy}-[2,3′-bipyridin]-6′- |
| | yl}cyclopropane-1-carboxamide (Isomer 3) |
| I-190 | (1S,2R)-2-[5-(difluoromethyl)pyridin-2-yl]-N-{5-[(1S)-1- |
| | hydroxyethyl]-6-{[(1R*,4R**,5S*)-2-methyl-2- |
| | azabicyclo[3.1.0]hexan-4-yl]oxy}-[2,3′-bipyridin]-6′- |
| | yl}cyclopropane-1-carboxamide (Isomer 4) |
| I-191 | (1S,2R)-2-(5-{bicyclo[1.1.1]pentan-1-yl}pyrimidin-2-yl)-N-{5- |
| | [(1S)-1-hydroxyethyl]-6-[4-methyl-1-(2,2,2-trifluoroethyl)-1H- |
| | pyrazol-3-yl]-[2,3′-bipyridin]-6′-yl}cyclopropane-1-carboxamide |
| I-192 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 1) |
| I-193 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 2) |
| I-194 | (1S,2R)-2-[5-(difluoromethyl)pyridin-2-yl]-N-[6-(1-ethyl-5- |
| | fluoro-3-methyl-1H-pyrazol-4-yl)-5-[(1S)-1-hydroxyethyl]-[2,3′- |
| | bipyridin]-6′-yl]cyclopropane-1-carboxamide |
| I-195 | (1S,2R)-2-[5-(difluoromethyl)pyrimidin-2-yl]-N-{5-[(1S)-1- |
| | hydroxyethyl]-6-(3-methyl-1-{spiro[2.2]pentan-1-yl}-1H- |
| | pyrazol-4-yl)-[2,3′-bipyridin]-6′-yl}cyclopropane-1-carboxamide |
| I-196 | (1S,2R)-N-{6-[(3R*,4R**)-1-azabicyclo[2.2.1]heptan-3-yloxy]- |
| | 5-[(1S)-1-hydroxyethyl]-[2,3′-bipyridin]-6′-yl}-2-[5- |
| | (difluoromethyl)pyridin-2-yl]cyclopropane-1-carboxamide |
| | (Isomer 3) |
| I-197 | (1S,2R)-N-{6-[(3R*,4R**)-1-azabicyclo[2.2.1]heptan-3-yloxy]- |
| | 5-[(1S)-1-hydroxyethyl]-[2,3′-bipyridin]-6′-yl}-2-[5- |
| | (difluoromethyl)pyridin-2-yl]cyclopropane-1-carboxamide |
| | (Isomer 1) |
| I-198 | (1S,2R)-N-{6-[(3R*,4R**)-1-azabicyclo[2.2.1]heptan-3-yloxy]- |
| | 5-[(1S)-1-hydroxyethyl]-[2,3′-bipyridin]-6′-yl}-2-[5- |
| | (difluoromethyl)pyridin-2-yl]cyclopropane-1-carboxamide |
| | (Isomer 2) |
| I-199 | (1S,2R)-2-(5-{bicyclo[1.1.1]pentan-1-yl}pyrimidin-2-yl)-N-{6- |
| | [1-(2,2-difluorocyclopropyl)-3-methyl-1H-pyrazol-4-yl]-5-[(1S)- |
| | 1-hydroxyethyl]-[2,3′-bipyridin]-6′-yl}cyclopropane-1- |
| | carboxamide |
| I-200 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | (5-methylpyrimidin-2-yl)cyclopropane-1-carboxamide (Isomer 1) |
| I-201 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | (5-methylpyrimidin-2-yl)cyclopropane-1-carboxamide (Isomer 2) |
| I-202 | (1S,2R)-2-(5-{bicyclo[1.1.1]pentan-1-yl}pyrimidin-2-yl)-N-(6- |
| | {1-[(1R*)-2,2-difluorocyclopropyl]-3-methyl-1H-pyrazol-4-yl}- |
| | 5-[(1S)-1-hydroxyethyl]-[2,3′-bipyridin]-6′-yl)cyclopropane-1- |
| | carboxamide (Isomer 1) |
| I-203 | (1S,2R)-2-(5-{bicyclo[1.1.1]pentan-1-yl}pyrimidin-2-yl)-N-(6- |
| | {1-[(1R*)-2,2-difluorocyclopropyl]-4-methyl-1H-pyrazol-3-yl}- |
| | 5-[(1S)-1-hydroxyethyl]-[2,3′-bipyridin]-6′-yl)cyclopropane-1- |
| | carboxamide (Isomer 2) |
| I-204 | (1S,2R)-2-(5-{bicyclo[1.1.1]pentan-1-yl}pyrimidin-2-yl)-N-(6- |
| | {1-[(1R*)-2,2-difluorocyclopropyl]-4-methyl-1H-pyrazol-3-yl}- |
| | 5-[(1S)-1-hydroxyethyl]-[2,3′-bipyridin]-6′-yl)cyclopropane-1- |
| | carboxamide (Isomer 1) |
| I-205 | (1S,2R)-2-(5-{bicyclo[1.1.1]pentan-1-yl}pyrimidin-2-yl)-N-(6- |
| | {1-[(1R*)-2,2-difluorocyclopropyl]-3-methyl-1H-pyrazol-4-yl}- |
| | 5-[(1S)-1-hydroxyethyl]-[2,3′-bipyridin]-6′-yl)cyclopropane-1- |
| | carboxamide (Isomer 2) |
| I-206 | (1S,2R)-2-[5-(difluoromethyl)pyrimidin-2-yl]-N-{5-[(1S)-1- |
| | hydroxyethyl]-6-(5-methyl-1-{spiro[2.2]pentan-1-yl}-1H- |
| | pyrazol-4-yl)-[2,3′-bipyridin]-6′-yl}cyclopropane-1-carboxamide |
| I-207 | (1S,2R)-2-[5-(difluoromethyl)pyridin-2-yl]-N-(6-{[(3S,5S)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 1) |
| I-208 | (1S,2R)-2-[5-(difluoromethyl)pyridin-2-yl]-N-(6-{[(3S,5S)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 2) |
| I-209 | (1S,2R)-2-[5-(difluoromethyl)pyridin-2-yl]-N-(6-{[(3R,5S)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 1) |
| I-210 | (1S,2R)-2-[5-(difluoromethyl)pyridin-2-yl]-N-(6-{[(3R,5S)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 2) |
| I-211 | (1S,2R)-2-[5-(difluoromethyl)pyridin-2-yl]-N-(6-{[(3S,5R)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 1) |
| I-212 | (1S,2R)-2-[5-(difluoromethyl)pyridin-2-yl]-N-(6-{[(3S,5R)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 2) |
| I-213 | (1S,2R)-2-[5-(difluoromethyl)pyridin-2-yl]-N-(6-{[(3R,5R)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 1) |
| I-214 | (1S,2R)-2-[5-(difluoromethyl)pyridin-2-yl]-N-(6-{[(3R,5R)-1,5- |
| | dimethylpyrrolidin-3-yl]oxy}-5-[(1R*)-1-hydroxyethyl]-[2,3′- |
| | bipyridin]-6′-yl)cyclopropane-1-carboxamide (Isomer 2) |
| I-215 | (1S,2R)-N-(6-{1-[(1R**)-2,2-difluorocyclopropyl]-3-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 1) |
| I-216 | (1S,2R)-N-(6-{1-[(1R**)-2,2-difluorocyclopropyl]-3-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 2) |
| I-217 | (1S,2R)-N-(6-{1-[(1R**)-2,2-difluorocyclopropyl]-3-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 3) |
| I-218 | (1S,2R)-N-(6-{1-[(1R**)-2,2-difluorocyclopropyl]-3-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 4) |
| I-219 | (1S,2R)-2-[5-(difluoromethyl)pyrimidin-2-yl]-N-{5-[(1S)-1- |
| | hydroxyethyl]-6-{3-methyl-1-[(1-methylcyclopropyl)methyl]- |
| | 1H-pyrazol-4-yl}-[2,3′-bipyridin]-6′-yl}cyclopropane-1- |
| | carboxamide |
| I-220 | (1S,2R)-2-[5-(difluoromethyl)pyrimidin-2-yl]-N-{5-[(1R)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-221 | (1S,2R)-2-[5-(difluoromethyl)pyrimidin-2-yl]-N-{5-[(1S)-1- |
| | hydroxyethyl]-6-{5-methyl-1-[(1-methylcyclopropyl)methyl]- |
| | 1H-pyrazol-4-yl}-[2,3′-bipyridin]-6′-yl}cyclopropane-1- |
| | carboxamide |
| I-222 | (1S,2R)-N-(6-{1-[(1R**)-2,2-difluorocyclopropyl]-5-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 1) |
| I-223 | (1S,2R)-N-(6-{1-[(1R**)-2,2-difluorocyclopropyl]-5-methyl-1H- |
| | pyrazol-4-yl}-4-fluoro-5-[(1R*)-1-hydroxyethyl]-[2,3′- |
| | bipyridin]-6′-yl)-2-(5-methylpyrimidin-2-yl)cyclopropane-1- |
| | carboxamide (Isomer 2) |
| I-224 | (1S,2R)-2-[5-(difluoromethyl)pyrimidin-2-yl]-N-{6-[1-(2-fluoro- |
| | 2-methylpropyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-225 | (1S,2R)-N-(6-{1-[(1S)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(trifluoromethyl)pyrimidin-2-yl]cyclopropane-1-carboxamide |
| | (Isomer 1) |
| I-226 | (1S,2R)-N-(6-{1-[(1R)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(trifluoromethyl)pyrimidin-2-yl]cyclopropane-1-carboxamide |
| | (Isomer 2) |
| I-227 | (1S,2R)-2-[5-(difluoromethyl)pyrimidin-2-yl]-N-{5- |
| | [(2R*,4R**)-4-hydroxyoxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 1) |
| I-228 | (1S,2R)-2-[5-(difluoromethyl)pyrimidin-2-yl]-N-{5- |
| | [(2R*,4R**)-4-hydroxyoxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 2) |
| I-229 | (1S,2R)-2-[5-(difluoromethyl)pyrimidin-2-yl]-N-{5- |
| | [(2R*,4R**)-4-hydroxyoxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 3) |
| I-230 | (1S,2R)-2-[5-(difluoromethyl)pyrimidin-2-yl]-N-{5- |
| | [(2R*,4R**)-4-hydroxyoxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 4) |
| I-231 | (1S,2R)-2-[5-(difluoromethyl)pyrimidin-2-yl]-N-{5-[(1S)-1- |
| | hydroxyethyl]-6-[1-(2,2,2-trifluoroethyl)-1H-pyrazol-4-yl]-[2,3′- |
| | bipyridin]-6′-yl}cyclopropane-1-carboxamide |
| I-232 | (1S,2R)-N-(6-{1-[(1R*)-2,2-difluorocyclopropyl]-1H-pyrazol-4- |
| | yl}-5-[(1S)-1-hydroxyethyl]-[2,3′-bipyridin]-6′-yl)-2-[5- |
| | (difluoromethyl)pyrimidin-2-yl]cyclopropane-1-carboxamide |
| | (Isomer 1) |
| I-233 | (1S,2R)-N-(6-{1-[(1R*)-2,2-difluorocyclopropyl]-1H-pyrazol-4- |
| | yl}-5-[(1S)-1-hydroxyethyl]-[2,3′-bipyridin]-6′-yl)-2-[5- |
| | (difluoromethyl)pyrimidin-2-yl]cyclopropane-1-carboxamide |
| | (Isomer 2) |
| I-234 | (1S,2R)-2-[5-(difluoromethyl)pyrimidin-2-yl]-N-{5-[(1S)-1- |
| | hydroxyethyl]-6-[3-methyl-1-(2-methylprop-2-en-1-yl)-1H- |
| | pyrazol-4-yl]-[2,3′-bipyridin]-6′-yl}cyclopropane-1-carboxamide |
| I-235 | (1S,2R)-2-[5-(difluoromethyl)pyridin-2-yl]-N-{5-[(2R*,4R**)-4- |
| | fluorooxolan-2-yl]-6-[3-methyl-1-(2,2,2-trifluoroethyl)-1H- |
| | pyrazol-4-yl]-[2,3′-bipyridin]-6′-yl}cyclopropane-1-carboxamide |
| | (Isomer 4) |
| I-236 | (1S,2R)-N-(6-{1-[(1R*)-2,2-difluorocyclopropyl]-5-methyl-1H- |
| | pyrazol-4-yl}-5-[(1S)-1-hydroxyethyl]-[2,3′-bipyridin]-6′-yl)-2- |
| | [5-(trifluoromethyl)pyrimidin-2-yl]cyclopropane-1-carboxamide |
| | (Isomer 3) |
| I-237 | (1S,2R)-N-(6-{1-[(1R*)-2,2-difluorocyclopropyl]-5-methyl-1H- |
| | pyrazol-4-yl}-5-[(1S)-1-hydroxyethyl]-[2,3′-bipyridin]-6′-yl)-2- |
| | [5-(trifluoromethyl)pyrimidin-2-yl]cyclopropane-1-carboxamide |
| | (Isomer 4) |
| I-238 | (1S,2R)-2-(6-cyclobutylpyridazin-3-yl)-N-(6-{1-[(1R*)-2,2- |
| | difluorocyclopropyl]-4-methyl-1H-pyrazol-3-yl}-5-[(1S)-1- |
| | hydroxyethyl]-[2,3′-bipyridin]-6′-yl)cyclopropane-1-carboxamide |
| | (Isomer 1) |
| I-239 | (1S,2R)-2-(6-cyclobutylpyridazin-3-yl)-N-(6-{1-[(1R*)-2,2- |
| | difluorocyclopropyl]-4-methyl-1H-pyrazol-3-yl}-5-[(1S)-1- |
| | hydroxyethyl]-[2,3′-bipyridin]-6′-yl)cyclopropane-1-carboxamide |
| | (Isomer 2) |
| I-240 | (1S,2R)-2-(5-{bicyclo[1.1.1]pentan-1-yl}pyrimidin-2-yl)-N-{5- |
| | [(1S)-1-hydroxyethyl]-6-{[(3S)-1-methylpyrrolidin-3-yl]oxy}- |
| | [2,3′-bipyridin]-6′-yl}cyclopropane-1-carboxamide |
| I-241 | (1S,2R)-2-(6-cyclobutylpyridazin-3-yl)-N-{6-[1-(2,2- |
| | difluorocyclopropyl)-4-methyl-1H-pyrazol-3-yl]-5-[(1S)-1- |
| | hydroxyethyl]-[2,3′-bipyridin]-6′-yl}cyclopropane-1- |
| | carboxamide |
| I-242 | (1S,2R)-2-(5-cyanopyridin-2-yl)-N-{6-[1-(2,2- |
| | difluorocyclopropyl)-4-methyl-1H-pyrazol-3-yl]-5-[(1S)-1- |
| | hydroxyethyl]-[2,3′-bipyridin]-6′-yl}cyclopropane-1- |
| | carboxamide |
| I-243 | (1S,2R)-2-[5-(difluoromethyl)pyrimidin-2-yl]-N-{5-[(1S)-1- |
| | hydroxyethyl]-6-[3-methyl-1-(3-methylbut-2-en-1-yl)-1H- |
| | pyrazol-4-yl]-[2,3′-bipyridin]-6′-yl}cyclopropane-1-carboxamide |
| I-244 | (1S,2R)-2-[5-(difluoromethyl)pyrimidin-2-yl]-N-{5-[(1S)-1- |
| | hydroxyethyl]-6-[5-methyl-1-(3-methylbut-2-en-1-yl)-1H- |
| | pyrazol-4-yl]-[2,3′-bipyridin]-6′-yl}cyclopropane-1-carboxamide |
| I-245 | (1S,2R)-N-{6-[3-(cyclopropyldifluoromethyl)-5-methyl-1H- |
| | pyrazol-1-yl]-5-[(1S)-1-hydroxyethyl]-[2,3′-bipyridin]-6′-yl}-2- |
| | [5-(difluoromethyl)pyrimidin-2-yl]cyclopropane-1-carboxamide |
| I-246 | (1S,2R)-2-[5-(difluoromethyl)pyrimidin-2-yl]-N-{5-[(1R*)-1- |
| | hydroxy-2-methoxyethyl]-6-[3-methyl-1-(2,2,2-trifluoroethyl)- |
| | 1H-pyrazol-4-yl]-[2,3′-bipyridin]-6′-yl}cyclopropane-1- |
| | carboxamide (Isomer 1) |
| I-247 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (3-fluoro-5-methylpyridin-2-yl)cyclopropane-1-carboxamide |
| | (Isomer 2) |
| I-248 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (3-fluoro-5-methylpyridin-2-yl)cyclopropane-1-carboxamide |
| | (Isomer 1) |
| I-249 | (1S,2R)-2-[5-(difluoromethyl)pyrimidin-2-yl]-N-{5-[(1R*)-1- |
| | hydroxy-2-methoxyethyl]-6-[3-methyl-1-(2,2,2-trifluoroethyl)- |
| | 1H-pyrazol-4-yl]-[2,3′-bipyridin]-6′-yl}cyclopropane-1- |
| | carboxamide |
| I-250 | (1S,2R)-N-{6-[5-(2,2-difluorocyclopropyl)-3-methyl-1H- |
| | pyrazol-1-yl]-5-[(1S)-1-hydroxyethyl]-[2,3′-bipyridin]-6′-yl}-2- |
| | [5-(difluoromethyl)pyrimidin-2-yl]cyclopropane-1-carboxamide |
| | (Isomer 1) |
| I-251 | (1S,2R)-N-{6-[3-(2,2-difluorocyclopropyl)-5-methyl-1H- |
| | pyrazol-1-yl]-5-[(1S)-1-hydroxyethyl]-[2,3′-bipyridin]-6′-yl}-2- |
| | [5-(difluoromethyl)pyrimidin-2-yl]cyclopropane-1-carboxamide |
| I-252 | (1S,2R)-2-[5-(1-fluoroethyl)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-253 | (1S,2R)-2-[5-(difluoromethyl)pyrimidin-2-yl]-N-{5-[(1S)-1- |
| | hydroxyethyl]-6-{3-methyl-1-[(1R*)-spiro[2.2]pentan-1-yl]-1H- |
| | pyrazol-4-yl}-[2,3′-bipyridin]-6′-yl}cyclopropane-1-carboxamide |
| | (Isomer 1) |
| I-254 | (1S,2R)-2-[5-(difluoromethyl)pyrimidin-2-yl]-N-{5-[(1S)-1- |
| | hydroxyethyl]-6-{3-methyl-1-[(1R*)-spiro[2.2]pentan-1-yl]-1H- |
| | pyrazol-4-yl}-[2,3′-bipyridin]-6′-yl}cyclopropane-1-carboxamide |
| | (Isomer 2) |
| I-255 | (1S,2R)-2-[5-(difluoromethyl)pyrimidin-2-yl]-N-{5- |
| | [(2R*,4R**)-4-fluorooxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 1) |
| I-256 | (1S,2R)-2-[5-(difluoromethyl)pyrimidin-2-yl]-N-{5- |
| | [(2R*,4R**)-4-fluorooxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 2) |
| I-257 | (1S,2R)-2-[5-(difluoromethyl)pyrimidin-2-yl]-N-{5- |
| | [(2R*,4R**)-4-fluorooxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 3) |
| I-258 | (1S,2R)-2-[5-(difluoromethyl)pyrimidin-2-yl]-N-{5- |
| | [(2R*,4R**)-4-fluorooxolan-2-yl]-6-[3-methyl-1-(2,2,2- |
| | trifluoroethyl)-1H-pyrazol-4-yl]-[2,3′-bipyridin]-6′- |
| | yl}cyclopropane-1-carboxamide (Isomer 4) |
| I-259 | (1S,2R)-N-(6-{3-[(1R*)-2,2-difluorocyclopropyl]-5-methyl-1H- |
| | pyrazol-1-yl}-5-[(1R**)-1-hydroxyethyl]-[2,3′-bipyridin]-6′-yl)- |
| | 2-[5-(difluoromethyl)pyrimidin-2-yl]cyclopropane-1- |
| | carboxamide (Isomer 1) |
| I-260 | (1S,2R)-N-(6-{3-[(1R*)-2,2-difluorocyclopropyl]-5-methyl-1H- |
| | pyrazol-1-yl}-5-[(1R**)-1-hydroxyethyl]-[2,3′-bipyridin]-6′-yl)- |
| | 2-[5-(difluoromethyl)pyrimidin-2-yl]cyclopropane-1- |
| | carboxamide (Isomer 2) |
| I-261 | (1S,2R)-N-(6-{3-[(1R*)-2,2-difluorocyclopropyl]-5-methyl-1H- |
| | pyrazol-1-yl}-5-[(1R**)-1-hydroxyethyl]-[2,3′-bipyridin]-6′-yl)- |
| | 2-[5-(difluoromethyl)pyrimidin-2-yl]cyclopropane-1- |
| | carboxamide (Isomer 3) |
| I-262 | (1S,2R)-N-(6-{3-[(1R*)-2,2-difluorocyclopropyl]-5-methyl-1H- |
| | pyrazol-1-yl}-5-[(1R**)-1-hydroxyethyl]-[2,3′-bipyridin]-6′-yl)- |
| | 2-[5-(difluoromethyl)pyrimidin-2-yl]cyclopropane-1- |
| | carboxamide (Isomer 4) |
| I-263 | (1S,2R)-2-{5-[(1R*)-1-fluoroethyl]pyrimidin-2-yl}-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide (Isomer |
| | 1) |
| I-264 | (1S,2R)-2-{5-[(1R*)-1-fluoroethyl]pyrimidin-2-yl}-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide (Isomer |
| | 2) |
| I-265 | (1S,2R)-2-[5-(1-fluorocyclopropyl)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-266 | (1S,2R)-2-[5-(difluoromethyl)pyrimidin-2-yl]-N-{6-[3-methyl-1- |
| | (2,2,2-trifluoroethyl)-1H-pyrazol-4-yl]-5-[(2R*)-oxolan-2-yl]- |
| | [2,3′-bipyridin]-6′-yl}cyclopropane-1-carboxamide (Isomer 1) |
| I-267 | (1S,2R)-2-[5-(difluoromethyl)pyrimidin-2-yl]-N-{6-[3-methyl-1- |
| | (2,2,2-trifluoroethyl)-1H-pyrazol-4-yl]-5-[(2R*)-oxolan-2-yl]- |
| | [2,3′-bipyridin]-6′-yl}cyclopropane-1-carboxamide (Isomer 2) |
| I-268 | (1S,2R)-2-[5-(difluoromethyl)pyrimidin-2-yl]-N-(6-{1-[(2R*)-2- |
| | fluoropropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-269 | (1S,2R)-2-[5-(difluoromethyl)pyrimidin-2-yl]-N-(6-{1-[(2R*)-2- |
| | fluoropropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-270 | (1S,2R)-N-(6-{1-[(1R*)-2,2-difluorocyclopropyl]-3-methyl-1H- |
| | pyrazol-4-yl}-5-[(2R**)-1,1-difluoropropan-2-yl]-[2,3′- |
| | bipyridin]-6′-yl)-2-[5-(difluoromethyl)pyrimidin-2- |
| | yl]cyclopropane-1-carboxamide (Isomer 1) |
| I-271 | (1S,2R)-N-(6-{1-[(1R*)-2,2-difluorocyclopropyl]-3-methyl-1H- |
| | pyrazol-4-yl}-5-[(2R**)-1,1-difluoropropan-2-yl]-[2,3′- |
| | bipyridin]-6′-yl)-2-[5-(difluoromethyl)pyrimidin-2- |
| | yl]cyclopropane-1-carboxamide (Isomer 3) |
| I-272 | (1S,2R)-N-(6-{1-[(1R*)-2,2-difluorocyclopropyl]-3-methyl-1H- |
| | pyrazol-4-yl}-5-[(2R**)-1,1-difluoropropan-2-yl]-[2,3′- |
| | bipyridin]-6′-yl)-2-[5-(difluoromethyl)pyrimidin-2- |
| | yl]cyclopropane-1-carboxamide (Isomer 4) |
| I-273 | (1S,2R)-N-(6-{1-[(1R*)-2,2-difluorocyclopropyl]-3-methyl-1H- |
| | pyrazol-4-yl}-5-[(2R**)-1,1-difluoropropan-2-yl]-[2,3′- |
| | bipyridin]-6′-yl)-2-[5-(difluoromethyl)pyrimidin-2- |
| | yl]cyclopropane-1-carboxamide (Isomer 2) |
| I-274 | (1S,2R)-2-[5-(difluoromethyl)pyridin-2-yl]-N-{6-[1-(2,2- |
| | difluoropropyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-275 | (1S,2R)-2-[5-(difluoromethyl)pyridin-2-yl]-N-{6-[1-(2,2- |
| | difluoropropyl)-5-methyl-1H-pyrazol-4-yl]-5-[(1S)-1- |
| | hydroxyethyl]-[2,3′-bipyridin]-6′-yl}cyclopropane-1- |
| | carboxamide |
| I-276 | (1S,2R)-2-[5-(difluoromethyl)pyrimidin-2-yl]-N-{6-[1-(2,2- |
| | difluoropropyl)-5-methyl-1H-pyrazol-4-yl]-5-[(1S)-1- |
| | hydroxyethyl]-[2,3′-bipyridin]-6′-yl}cyclopropane-1- |
| | carboxamide |
| I-277 | 3-[(1R)-1-[5-(difluoromethyl)pyridin-2-yl]ethyl]-1-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}urea |
| I-278 | (1S,2R)-2-[5-(difluoromethyl)pyridin-2-yl]-N-{5-[(1R*)-1- |
| | hydroxyethyl]-6-methoxy-4′-methyl-[2,3′-bipyridin]-6′- |
| | yl}cyclopropane-1-carboxamide (Isomer 1) |
| I-279 | (1S,2R)-N-[6-(1-cyclopropyl-3-fluoro-5-methyl-1H-pyrazol-4- |
| | yl)-5-[(1S)-1-hydroxyethyl]-[2,3′-bipyridin]-6′-yl]-2-[5- |
| | (difluoromethyl)pyrimidin-2-yl]cyclopropane-1-carboxamide |
| I-280 | N-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-2-(5-methylpyridin-2- |
| | yl)acetamide |
| I-281 | N-{6-[1-(2-fluoro-2-methylpropyl)-3-methyl-1H-pyrazol-4-yl]-5- |
| | [(1S)-1-hydroxyethyl]-[2,3′-bipyridin]-6′-yl}-2-(5-methylpyridin- |
| | 2-yl)acetamide |
| I-282 | N-{6-[1-(2-fluoro-2-methylpropyl)-5-methyl-1H-pyrazol-4-yl]-5- |
| | [(1S)-1-hydroxyethyl]-[2,3′-bipyridin]-6′-yl}-2-(5-methylpyridin- |
| | 2-yl)acetamide |
| I-283 | (1S,2R)-2-[5-(difluoromethyl)pyridin-2-yl]-N-{5-[(1R*)-1- |
| | hydroxyethyl]-6-methoxy-4′-methyl-[2,3′-bipyridin]-6′- |
| | yl}cyclopropane-1-carboxamide (Isomer 2) |
| I-284 | (1S,2R)-N-{6-[1-(2-fluoro-2-methylpropyl)-4-methyl-1H- |
| | pyrazol-3-yl]-5-[(1S)-1-hydroxyethyl]-[2,3′-bipyridin]-6′-yl}-2- |
| | (5-methylpyrimidin-2-yl)cyclopropane-1-carboxamide |
| I-285 | N-(6-{1-[(1R*)-2,2-difluorocyclopropyl]-3-methyl-1H-pyrazol- |
| | 4-yl}-5-[(1S)-1-hydroxyethyl]-[2,3′-bipyridin]-6′-yl)-2-(5- |
| | methylpyridin-2-yl)acetamide (Isomer 1) |
| I-286 | N-(6-{1-[(1R*)-2,2-difluorocyclopropyl]-3-methyl-1H-pyrazol- |
| | 4-yl}-5-[(1S)-1-hydroxyethyl]-[2,3′-bipyridin]-6′-yl)-2-(5- |
| | methylpyridin-2-yl)acetamide (Isomer 2) |
| I-287 | (1S,2R)-N-{6-[1-(2-fluoro-2-methylpropyl)-3-methyl-1H- |
| | pyrazol-4-yl]-5-[(1S)-1-hydroxyethyl]-[2,3′-bipyridin]-6′-yl}-2- |
| | [5-(trifluoromethyl)pyridin-2-yl]cyclopropane-1-carboxamide |
| I-288 | (1S,2R)-N-{6-[1-(2-fluoro-2-methylpropyl)-5-methyl-1H- |
| | pyrazol-4-yl]-5-[(1S)-1-hydroxyethyl]-[2,3′-bipyridin]-6′-yl}-2- |
| | [5-(trifluoromethyl)pyridin-2-yl]cyclopropane-1-carboxamide |
| I-289 | (1S,2R)-2-[5-(difluoromethyl)pyrimidin-2-yl]-N-{6-[1-(2,2- |
| | dimethylcyclopropyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-290 | (1S,2R)-2-[5-(difluoromethyl)pyrimidin-2-yl]-N-(6-{1-[(1R*)- |
| | 2,2-dimethylcyclopropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-291 | (1S,2R)-2-[5-(difluoromethyl)pyrimidin-2-yl]-N-(6-{1-[(1R*)- |
| | 2,2-dimethylcyclopropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-292 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyrazin-2-yl]cyclopropane-1-carboxamide |
| | (Isomer 1) |
| I-293 | (1S,2R)-N-(6-{1-[(1R*)-2,2-difluorocyclopropyl]-4-methyl-1H- |
| | pyrazol-3-yl}-5-[(1S)-1-hydroxyethyl]-[2,3′-bipyridin]-6′-yl)-2- |
| | [5-(difluoromethyl)pyrazin-2-yl]cyclopropane-1-carboxamide |
| | (Isomer 2) |
| I-294 | (1S,2R)-2-[5-(difluoromethyl)pyrimidin-2-yl]-N-(6-{1-[(1R*)- |
| | 2,2-dimethylcyclopropyl]-5-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-295 | (1S,2R)-2-[5-(difluoromethyl)pyrimidin-2-yl]-N-(6-{1-[(1R*)- |
| | 2,2-dimethylcyclopropyl]-5-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-296 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (difluoromethyl)pyridin-2-yl]ethyl]urea (Isomer 1) |
| I-297 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (difluoromethyl)pyridin-2-yl]ethyl]urea (Isomer 2) |
| I-298 | 3-(6-{1-[(1R*)-2,2-difluorocyclopropyl]-4-methyl-1H-pyrazol-3- |
| | yl}-5-[(1S)-1-hydroxyethyl]-[2,3′-bipyridin]-6′-yl)-1-[(1R)-1-[5- |
| | (difluoromethyl)pyridin-2-yl]ethyl]urea (Isomer 1) |
| I-299 | 3-(6-{1-[(1R*)-2,2-difluorocyclopropyl]-4-methyl-1H-pyrazol-3- |
| | yl}-5-[(1S)-1-hydroxyethyl]-[2,3′-bipyridin]-6′-yl)-1-[(1R)-1-[5- |
| | (difluoromethyl)pyridin-2-yl]ethyl]urea (Isomer 2) |
| I-300 | 3-(6-{1-[(1R*)-2,2-difluorocyclopropyl]-4-methyl-1H-pyrazol-3- |
| | yl}-5-[(1S)-1-hydroxyethyl]-[2,3′-bipyridin]-6′-yl)-1-[(1R)-1-(5- |
| | methylpyrimidin-2-yl)ethyl]urea (Isomer 1) |
| I-301 | 3-(6-{1-[(1R*)-2,2-difluorocyclopropyl]-4-methyl-1H-pyrazol-3- |
| | yl}-5-[(1S)-1-hydroxyethyl]-[2,3′-bipyridin]-6′-yl)-1-[(1R)-1-(5- |
| | methylpyrimidin-2-yl)ethyl]urea (Isomer 2) |
| I-302 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (trifluoromethyl)pyrazin-2-yl]ethyl]urea (Isomer 1) |
| I-303 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (trifluoromethyl)pyrazin-2-yl]ethyl]urea (Isomer 2) |
| I-304 | N-{6-[1-(2-fluoro-2-methylpropyl)-3-methyl-1H-pyrazol-4-yl]-5- |
| | [(1S)-1-hydroxyethyl]-[2,3′-bipyridin]-6′-yl}-2-{5H,6H,8H- |
| | imidazo[2,1-c][1,4]oxazin-2-yl}acetamide |
| I-305 | N-{6-[1-(2-fluoro-2-methylpropyl)-5-methyl-1H-pyrazol-4-yl]-5- |
| | [(1S)-1-hydroxyethyl]-[2,3′-bipyridin]-6′-yl}-2-{5H,6H,8H- |
| | imidazo[2,1-c][1,4]oxazin-2-yl}acetamide |
| I-306 | (1S,2R)-N-{6-[3-(difluoromethoxy)-5-methyl-1H-pyrazol-1-yl]- |
| | 5-[(1R*)-1-hydroxyethyl]-[2,3′-bipyridin]-6′-yl}-2-[5- |
| | (difluoromethyl)pyrimidin-2-yl]cyclopropane-1-carboxamide |
| | (Isomer 1) |
| I-307 | (1S,2R)-N-{6-[3-(difluoromethoxy)-5-methyl-1H-pyrazol-1-yl]- |
| | 5-[(1R*)-1-hydroxyethyl]-[2,3′-bipyridin]-6′-yl}-2-[5- |
| | (difluoromethyl)pyrimidin-2-yl]cyclopropane-1-carboxamide |
| | (Isomer 2) |
| I-308 | (1S,2R)-2-[5-(difluoromethyl)pyridin-2-yl]-N-[6-(hexahydro-1H- |
| | pyrrolizin-1-yloxy)-5-[(1S)-1-hydroxyethyl]-[2,3′-bipyridin]-6′- |
| | yl]cyclopropane-1-carboxamide |
| I-309 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5- |
| | (trifluoromethyl)pyridin-2-yl]cyclopropane-1-carboxamide |
| I-310 | (1S,2R)-2-[5-(difluoromethyl)pyrimidin-2-yl]-N-{6-[3-(1- |
| | fluorocyclopropyl)-5-methyl-1H-pyrazol-1-yl]-5-[(1R*)-1- |
| | hydroxyethyl]-[2,3′-bipyridin]-6′-yl}cyclopropane-1- |
| | carboxamide (Isomer 2) |
| I-311 | (1S,2R)-2-[5-(difluoromethyl)pyridin-2-yl]-N-{5-[(1R*)-1- |
| | hydroxyethyl]-6-methoxy-4′-(propan-2-yl)-[2,3′-bipyridin]-6′- |
| | yl}cyclopropane-1-carboxamide (Isomer 1) |
| I-312 | (1S,2R)-2-[5-(difluoromethyl)pyridin-2-yl]-N-{5-[(1R*)-1- |
| | hydroxyethyl]-6-methoxy-4′-(propan-2-yl)-[2,3′-bipyridin]-6′- |
| | yl}cyclopropane-1-carboxamide (Isomer 2) |
| I-313 | (1S,2R)-N-[6-(1-cyclopropyl-3-methyl-1H-pyrazol-4-yl)-5-[(1S)- |
| | 1-hydroxyethyl]-[2,3′-bipyridin]-6′-yl]-2-[5- |
| | (difluoromethyl)pyridin-2-yl]cyclopropane-1-carboxamide |
| I-314 | (1S,2R)-N-[6-(1-cyclopropyl-5-methyl-1H-pyrazol-4-yl)-5-[(1S)- |
| | 1-hydroxyethyl]-[2,3′-bipyridin]-6′-yl]-2-[5- |
| | (difluoromethyl)pyridin-2-yl]cyclopropane-1-carboxamide |
| I-315 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(trifluoromethyl)pyridin-2-yl]cyclopropane-1-carboxamide |
| | (Isomer 1) |
| I-316 | (1S,2R)-N-{6-[1-(2-fluoro-2-methylpropyl)-3-methyl-1H- |
| | pyrazol-4-yl]-5-[(1S)-1-hydroxyethyl]-[2,3′-bipyridin]-6′-yl}-2- |
| | [5-(trifluoromethyl)pyrimidin-2-yl]cyclopropane-1-carboxamide |
| I-317 | (1S,2R)-N-{6-[1-(2-fluoro-2-methylpropyl)-5-methyl-1H- |
| | pyrazol-4-yl]-5-[(1S)-1-hydroxyethyl]-[2,3′-bipyridin]-6′-yl}-2- |
| | [5-(trifluoromethyl)pyrimidin-2-yl]cyclopropane-1-carboxamide |
| I-318 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (difluoromethyl)pyrazin-2-yl]ethyl]urea (Isomer 1) |
| I-319 | 1-(6-{1-[(1R*)-2,2-difluorocyclopropyl]-3-methyl-1H-pyrazol-4- |
| | yl}-5-[(1S)-1-hydroxyethyl]-[2,3′-bipyridin]-6′-yl)-3-[(1R)-1-[5- |
| | (difluoromethyl)pyrazin-2-yl]ethyl]urea (Isomer 2) |
| I-320 | 3-(6-{1-[(1R**)-2,2-difluorocyclopropyl]-4-methyl-1H-pyrazol- |
| | 3-yl}-5-[(1S)-1-hydroxyethyl]-[2,3′-bipyridin]-6′-yl)-1-[(1R*)-1- |
| | [5-(difluoromethyl)pyrimidin-2-yl]ethyl]urea (Isomer 1) |
| I-321 | 3-(6-{1-[(1R**)-2,2-difluorocyclopropyl]-4-methyl-1H-pyrazol- |
| | 3-yl}-5-[(1S)-1-hydroxyethyl]-[2,3′-bipyridin]-6′-yl)-1-[(1R*)-1- |
| | [5-(difluoromethyl)pyrimidin-2-yl]ethyl]urea (Isomer 2) |
| I-322 | 3-(6-{1-[(1R**)-2,2-difluorocyclopropyl]-4-methyl-1H-pyrazol- |
| | 3-yl}-5-[(1S)-1-hydroxyethyl]-[2,3′-bipyridin]-6′-yl)-1-[(1R*)-1- |
| | [5-(difluoromethyl)pyrimidin-2-yl]ethyl]urea (Isomer 3) |
| I-323 | 3-(6-{1-[(1R**)-2,2-difluorocyclopropyl]-4-methyl-1H-pyrazol- |
| | 3-yl}-5-[(1S)-1-hydroxyethyl]-[2,3′-bipyridin]-6′-yl)-1-[(1R*)-1- |
| | [5-(difluoromethyl)pyrimidin-2-yl]ethyl]urea (Isomer 4) |
| I-324 | 2-(1,5-dimethyl-1H-pyrazol-3-yl)-N-{6-[1-(2-fluoro-2- |
| | methylpropyl)-3-methyl-1H-pyrazol-4-yl]-5-[(1S)-1- |
| | hydroxyethyl]-[2,3′-bipyridin]-6′-yl}acetamide |
| I-325 | 2-(1,5-dimethyl-1H-pyrazol-3-yl)-N-{6-[1-(2-fluoro-2- |
| | methylpropyl)-5-methyl-1H-pyrazol-4-yl]-5-[(1S)-1- |
| | hydroxyethyl]-[2,3′-bipyridin]-6′-yl}acetamide |
| I-326 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5-{2- |
| | oxabicyclo[2.1.1]hexan-4-yl}pyridin-2-yl)cyclopropane-1- |
| | carboxamide |
| I-327 | (1S,2R)-2-[5-(3,3-difluoroazetidin-1-yl)pyrazin-2-yl]-N-{5- |
| | [(1S)-1-hydroxyethyl]-6-[3-methyl-1-(2,2,2-trifluoroethyl)-1H- |
| | pyrazol-4-yl]-[2,3′-bipyridin]-6′-yl}cyclopropane-1-carboxamide |
| I-328 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)pyrazin-2-yl]cyclopropane-1-carboxamide |
| | (Isomer 2) |
| I-329 | 3-[6-(3-cyano-5-methyl-1H-pyrazol-1-yl)-5-[(1S)-1- |
| | hydroxyethyl]-[2,3′-bipyridin]-6′-yl]-1-[(1R)-1-[5- |
| | (difluoromethyl)pyrimidin-2-yl]ethyl]urea |
| I-330 | (1S,2R)-N-[6-(1-{[1-(difluoromethyl)cyclopropyl]methyl}-3- |
| | methyl-1H-pyrazol-4-yl)-5-[(1S)-1-hydroxyethyl]-[2,3′- |
| | bipyridin]-6′-yl]-2-[5-(difluoromethyl)pyrimidin-2- |
| | yl]cyclopropane-1-carboxamide |
| I-331 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5-{1- |
| | methyl-2-oxabicyclo[2.1.1]hexan-4-yl}pyrimidin-2- |
| | yl)cyclopropane-1-carboxamide |
| I-332 | (1S,2R)-2-(5-{3-fluorobicyclo[1.1.1]pentan-1-yl}pyrimidin-2- |
| | yl)-N-{5-[(1S)-1-hydroxyethyl]-6-[3-methyl-1-(2,2,2- |
| | trifluoroethyl)-1H-pyrazol-4-yl]-[2,3′-bipyridin]-6′- |
| | yl}cyclopropane-1-carboxamide |
| I-333 | (1S,2R)-N-{6-[3-(2,2-difluoroethyl)-1-(2,2,2-trifluoroethyl)-1H- |
| | pyrazol-4-yl]-5-[(1S)-1-hydroxyethyl]-[2,3′-bipyridin]-6′-yl}-2- |
| | [5-(difluoromethyl)pyrimidin-2-yl]cyclopropane-1-carboxamide |
| I-334 | (1S,2R)-N-[6-(1-{[(1R)-2,2-difluorocyclopropyl]methyl}-3- |
| | methyl-1H-pyrazol-4-yl)-5-[(1S)-1-hydroxyethyl]-[2,3′- |
| | bipyridin]-6′-yl]-2-[5-(difluoromethyl)pyrimidin-2- |
| | yl]cyclopropane-1-carboxamide |
| I-335 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5-(1- |
| | methylazetidin-2-yl)pyrimidin-2-yl]cyclopropane-1-carboxamide |
| I-336 | N-(6-{1-[(1R*)-2,2-difluorocyclopropyl]-3-methyl-1H-pyrazol- |
| | 4-yl}-5-[(1S)-1-hydroxyethyl]-[2,3′-bipyridin]-6′-yl)-2- |
| | {5H,6H,7H-pyrazolo[3,2-b][1,3]oxazin-2-yl}acetamide (Isomer |
| | 2) |
| I-337 | (1S,2R)-2-[5-(difluoromethyl)pyrimidin-2-yl]-N-[6-(1-{3- |
| | fluorobicyclo[1.1.1]pentan-1-yl}-3-methyl-1H-pyrazol-4-yl)-5- |
| | [(1S)-1-hydroxyethyl]-[2,3′-bipyridin]-6′-yl]cyclopropane-1- |
| | carboxamide |
| I-338 | (1S,2R)-2-[5-(difluoromethyl)pyridin-2-yl]-N-{20-[(1S)-1- |
| | hydroxyethyl]-4-(2,2,2-trifluoroethyl)-4,5,10,14,21- |
| | pentaazatetracyclo[15.3.1.0\hat{ }{2,6}.0\hat{ }{11,16}]henicosa- |
| | 1(21),2,5,11,13,15,17,19-octaen-13-yl}cyclopropane-1- |
| | carboxamide |
| I-339 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(1,1-difluoroethyl)pyrimidin-2-yl]cyclopropane-1- |
| | carboxamide (Isomer 2) |
| I-340 | N-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-2-(1-methyl-1H-pyrazol- |
| | 3-yl)acetamide |
| I-341 | N-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-2-{5H,6H,7H- |
| | pyrazolo[3,2-b][1,3]oxazin-2-yl}acetamide |
| I-342 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{5- |
| | [(2R)-2-methoxycyclobutyl]pyridin-2-yl}cyclopropane-1- |
| | carboxamide |
| I-343 | (1S,2R)-N-{5-[(1S)-1-hydroxyethyl]-6-[5-methyl-2- |
| | (trifluoromethyl)pyrimidin-4-yl]-[2,3′-bipyridin]-6′-yl}-2-(5- |
| | methylpyrimidin-2-yl)cyclopropane-1-carboxamide |
| I-344 | (1S,2R)-2-[5-(difluoromethyl)pyrimidin-2-yl]-N-(6-{1-[(1S,2S)- |
| | 2-fluorocyclopropyl]-3-methyl-1H-pyrazol-4-yl}-5-[(1S)-1- |
| | hydroxyethyl]-[2,3′-bipyridin]-6′-yl)cyclopropane-1-carboxamide |
| I-345 | (6aR*,7S*,7aS*)-N-{5-[(1S)-1-hydroxyethyl]-6-[3-methyl-1- |
| | (2,2,2-trifluoroethyl)-1H-pyrazol-4-yl]-[2,3′-bipyridin]-6′-yl}- |
| | 5H,6H,6aH,7H,7aH-cyclopropa[h]quinoline-7-carboxamide |
| | (Isomer 1) |
| I-346 | (6aR*,7S*,7aS*)-N-{5-[(1S)-1-hydroxyethyl]-6-[3-methyl-1- |
| | (2,2,2-trifluoroethyl)-1H-pyrazol-4-yl]-[2,3′-bipyridin]-6′-yl}- |
| | 5H,6H,6aH,7H,7aH-cyclopropa[h]quinoline-7-carboxamide |
| | (Isomer 2) |
| I-347 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(trifluoromethyl)pyrazin-2-yl]cyclopropane-1-carboxamide |
| | (Isomer 2) |
| I-348 | (1S,2R)-N-[5-(difluoromethoxy)-6-[3-methyl-1-(2,2,2- |
| | trifluoroethyl)-1H-pyrazol-4-yl]-[2,3′-bipyridin]-6′-yl]-2-[5- |
| | (difluoromethyl)pyrimidin-2-yl]cyclopropane-1-carboxamide |
| I-349 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5- |
| | (pentafluoro-lambda6-sulfanyl)pyridin-2-yl]cyclopropane-1- |
| | carboxamide |
| I-350 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)-3-fluoropyridin-2-yl]cyclopropane-1- |
| | carboxamide (Isomer 2) |
| I-351 | (1S,2R)-2-[5-(difluoromethyl)-3-fluoropyridin-2-yl]-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide |
| I-352 | (1S,2R)-2-[5-(difluoromethyl)pyridin-2-yl]-N-{5-[(1S)-1- |
| | hydroxyethyl]-6-methoxy-4′-(methylamino)-[2,3′-bipyridin]-6′- |
| | yl}cyclopropane-1-carboxamide |
| I-353 | N-(6-{1-[(1R*)-2,2-difluorocyclopropyl]-3-methyl-1H-pyrazol- |
| | 4-yl}-5-[(1S)-1-hydroxyethyl]-[2,3′-bipyridin]-6′-yl)-2-(2- |
| | methyl-1,3-thiazol-4-yl)acetamide (Isomer 2) |
| I-354 | (1S,2R)-2-{5-[1-(dimethylamino)cyclopropyl]pyrazin-2-yl}-N- |
| | {5-[(1S)-1-hydroxyethyl]-6-[3-methyl-1-(2,2,2-trifluoroethyl)- |
| | 1H-pyrazol-4-yl]-[2,3′-bipyridin]-6′-yl}cyclopropane-1- |
| | carboxamide |
| I-355 | (1S,2R)-2-{5-[1-(dimethylamino)cyclopropyl]pyrimidin-2-yl}-N- |
| | {5-[(1S)-1-hydroxyethyl]-6-[3-methyl-1-(2,2,2-trifluoroethyl)- |
| | 1H-pyrazol-4-yl]-[2,3′-bipyridin]-6′-yl}cyclopropane-1- |
| | carboxamide |
| I-356 | (1R*)-1-[5-(difluoromethyl)pyrimidin-2-yl]ethyl N-(6-{1- |
| | [(1R**)-2,2-difluorocyclopropyl]-3-methyl-1H-pyrazol-4-yl}-5- |
| | [(1S)-1-hydroxyethyl]-[2,3′-bipyridin]-6′-yl)carbamate (Isomer 1) |
| I-357 | (1R*)-1-[5-(difluoromethyl)pyrimidin-2-yl]ethyl N-(6-{1- |
| | [(1R**)-2,2-difluorocyclopropyl]-3-methyl-1H-pyrazol-4-yl}-5- |
| | [(1S)-1-hydroxyethyl]-[2,3′-bipyridin]-6′-yl)carbamate (Isomer 2) |
| I-358 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | {5-[1-(dimethylamino)cyclopropyl]pyrazin-2-yl}cyclopropane-1- |
| | carboxamide (Isomer 2) |
| I-359 | (1S,2R)-2-[5-(difluoromethyl)pyridin-2-yl]-N-{15-[(1S)-1- |
| | hydroxyethyl]-13-oxa-4,8,18- |
| | triazatricyclo[12.3.1.0\hat{ }{2,7}]octadeca-1(17),2,4,6,14(18),15- |
| | hexaen-5-yl}cyclopropane-1-carboxamide |
| I-360 | (1R*)-1-[5-(difluoromethyl)pyrimidin-2-yl]ethyl N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}carbamate (Isomer 1) |
| I-361 | (1R*)-1-[5-(difluoromethyl)pyrimidin-2-yl]ethyl N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}carbamate (Isomer 2) |
| I-362 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | {5-[1-(dimethylamino)cyclopropyl]pyrimidin-2- |
| | yl}cyclopropane-1-carboxamide |
| I-363 | (1S,2R)-N-{6-[3-(cyanomethyl)-5-methyl-1H-pyrazol-1-yl]-5- |
| | [(1S)-1-hydroxyethyl]-[2,3′-bipyridin]-6′-yl}-2-[5- |
| | (difluoromethyl)pyrimidin-2-yl]cyclopropane-1-carboxamide |
| I-364 | (1S,2R)-N-(6-{2-[(1R*)-2,2-difluorocyclopropyl]-5-methyl-2H- |
| | 1,2,3-triazol-4-yl}-5-[(1S)-1-hydroxyethyl]-[2,3′-bipyridin]-6′- |
| | yl)-2-[5-(difluoromethyl)pyrimidin-2-yl]cyclopropane-1- |
| | carboxamide (Isomer 1) |
| I-365 | (1S,2R)-N-(6-{2-[(1R*)-2,2-difluorocyclopropyl]-5-methyl-2H- |
| | 1,2,3-triazol-4-yl}-5-[(1S)-1-hydroxyethyl]-[2,3′-bipyridin]-6′- |
| | yl)-2-[5-(difluoromethyl)pyrimidin-2-yl]cyclopropane-1- |
| | carboxamide (Isomer 2) |
| I-366 | (1S,2R)-N-{6-[1-(2,2-difluorocyclopropyl)-5-methyl-1H-1,2,3- |
| | triazol-4-yl]-5-[(1S)-1-hydroxyethyl]-[2,3′-bipyridin]-6′-yl}-2-[5- |
| | (difluoromethyl)pyrimidin-2-yl]cyclopropane-1-carboxamide |
| I-367 | (1S,2R)-2-[5-(difluoromethyl)pyridin-2-yl]-N-[4′- |
| | (dimethylamino)-5-[(1S)-1-hydroxyethyl]-6-methoxy-[2,3′- |
| | bipyridin]-6′-yl]cyclopropane-1-carboxamide |
| I-368 | (1S,2R)-2-[5-(difluoromethyl)pyridin-2-yl]-N-(5-{4-[(1R*)-1- |
| | hydroxyethyl]-3-methoxyphenyl}-4-methoxypyridin-2- |
| | yl)cyclopropane-1-carboxamide (Isomer 1) |
| I-369 | (1S,2R)-2-[5-(difluoromethyl)pyridin-2-yl]-N-(5-{4-[(1R*)-1- |
| | hydroxyethyl]-3-methoxyphenyl}-4-methoxypyridin-2- |
| | yl)cyclopropane-1-carboxamide (Isomer 2) |
| I-370 | (1S,2R)-N-(6-{1-[(1R*)-2,2-difluorocyclobutyl]-3-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 1) |
| I-371 | (1S,2R)-N-(6-{1-[(1R*)-2,2-difluorocyclobutyl]-3-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 2) |
| I-372 | (1S,2R)-2-[5-(difluoromethyl)pyridin-2-yl]-N-{4′-acetamido-5- |
| | [(1S)-1-hydroxyethyl]-6-methoxy-[2,3′-bipyridin]-6′- |
| | yl}cyclopropane-1-carboxamide |
| I-373 | (1S,2R)-2-[5-(difluoromethyl)pyridin-2-yl]-N-{5-[(1S)-1- |
| | hydroxyethyl]-6-methoxy-4′-[(oxetan-3-yl)amino]-[2,3′- |
| | bipyridin]-6′-yl}cyclopropane-1-carboxamide |
| I-374 | (1S,2R)-2-[5-(difluoromethyl)pyridin-2-yl]-N-{5-[(1S)-1- |
| | hydroxyethyl]-6-methoxy-4′-{[(oxetan-3-yl)methyl]amino}-[2,3′- |
| | bipyridin]-6′-yl}cyclopropane-1-carboxamide |
| I-375 | (1S,2R)-N-[6-(difluoromethoxy)-5-[(1R*)-1-hydroxyethyl]-4′- |
| | (methylamino)-[2,3′-bipyridin]-6′-yl]-2-[5- |
| | (difluoromethyl)pyridin-2-yl]cyclopropane-1-carboxamide (peak |
| | 1 in SFC) |
| I-376 | (1S,2R)-N-[6-(difluoromethoxy)-5-[(1R*)-1-hydroxyethyl]-4′- |
| | (methylamino)-[2,3′-bipyridin]-6′-yl]-2-[5- |
| | (difluoromethyl)pyridin-2-yl]cyclopropane-1-carboxamide (peak |
| | 2 in SFC) |
| I-377 | (1S,2R)-N-[4′-(ethylamino)-5-[(1S)-1-hydroxyethyl]-6-methoxy- |
| | [2,3′-bipyridin]-6′-yl]-2-(5-{3-fluorobicyclo[1.1.1]pentan-1- |
| | yl}pyrimidin-2-yl)cyclopropane-1-carboxamide |
| I-378 | (1S,2R)-N-{5-acetyl-6-[3-methyl-1-(2,2,2-trifluoroethyl)-1H- |
| | pyrazol-4-yl]-[2,3′-bipyridin]-6′-yl}-2-[5- |
| | (difluoromethyl)pyrimidin-2-yl]cyclopropane-1-carboxamide |
| I-379 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5- |
| | (hydroxymethyl)pyrimidin-2-yl]cyclopropane-1-carboxamide |
| I-380 | 2-[(1R,2S)-2-({5-[(1S)-1-hydroxyethyl]-6-[3-methyl-1-(2,2,2- |
| | trifluoroethyl)-1H-pyrazol-4-yl]-[2,3′-bipyridin]-6′- |
| | yl}carbamoyl)cyclopropyl]pyrimidine-5-carboxylic acid |
| I-381 | (1S,2R)-2-[5-(3,3-difluoroazetidin-1-yl)pyridin-2-yl]-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide |
| I-382 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5-{2- |
| | oxabicyclo[2.1.1]hexan-1-yl}pyridin-2-yl)cyclopropane-1- |
| | carboxamide |
| I-383 | (1S,2R)-N-{5-[(1S)-1-hydroxyethyl]-6-[5-methyl-1-(2,2,2- |
| | trifluoroethyl)-1H-1,2,3-triazol-4-yl]-[2,3′-bipyridin]-6′-yl}-2-(5- |
| | {2-oxabicyclo[2.1.1]hexan-1-yl}pyridin-2-yl)cyclopropane-1- |
| | carboxamide |
| I-384 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-(5-{2- |
| | oxabicyclo[2.1.1]hexan-1-yl}pyrimidin-2-yl)cyclopropane-1- |
| | carboxamide |
| I-385 | (1S,2R)-2-[5-(3-fluoroazetidin-1-yl)pyridin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-386 | (1S,2R)-N-{5-[(1S)-1-hydroxyethyl]-6-[5-methyl-1-(2,2,2- |
| | trifluoroethyl)-1H-1,2,3-triazol-4-yl]-[2,3′-bipyridin]-6′-yl}-2-(5- |
| | {2-oxabicyclo[2.1.1]hexan-4-yl}pyridin-2-yl)cyclopropane-1- |
| | carboxamide |
| I-387 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5- |
| | (propan-2-yloxy)pyrimidin-2-yl]cyclopropane-1-carboxamide |
| I-388 | (1S,2R)-2-[5-(fluoromethyl)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-389 | (1S,2R)-N-(6-{1-[(1R*)-2,2-difluorocyclobutyl]-3-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 1) |
| I-390 | (1S,2R)-N-(6-{1-[(1R*)-2,2-difluorocyclobutyl]-5-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 2) |
| I-391 | (1S,2R)-2-[5-(difluoromethyl)pyrimidin-2-yl]-N-(6-{1-[(1S,2R)- |
| | 2-fluorocyclopropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-392 | (1S,2R)-2-[5-(difluoromethyl)pyrimidin-2-yl]-N-(6-{1-[(1S,2R)- |
| | 2-fluorocyclopropyl]-5-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-393 | (1S,2R)-N-(6-{1-[(1R*)-2,2-difluorocyclobutyl]-5-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 1) |
| I-394 | (1S,2R)-N-(6-{1-[(1R*)-2,2-difluorocyclobutyl]-3-methyl-1H- |
| | pyrazol-4-yl}-5-[(1S)-1-hydroxyethyl]-[2,3′-bipyridin]-6′-yl)-2- |
| | [5-(difluoromethyl)pyrimidin-2-yl]cyclopropane-1-carboxamide |
| | (Isomer 2) |
| I-395 | (1S,2R)-N-(6-{1-[(1R*)-2,2-difluorocyclopropyl]-3-methyl-1H- |
| | pyrazol-4-yl}-5-(difluoromethoxy)-[2,3′-bipyridin]-6′-yl)-2-(5- |
| | methylpyrimidin-2-yl)cyclopropane-1-carboxamide |
| I-396 | 1-(6-{1-[(1R**)-2,2-difluorocyclopropyl]-3-methyl-1H-pyrazol- |
| | 4-yl}-5-(difluoromethoxy)-[2,3′-bipyridin]-6′-yl)-3-[(1R*)-1-(5- |
| | methylpyrimidin-2-yl)ethyl]urea (Isomer 1) |
| I-397 | 1-(6-{1-[(1R**)-2,2-difluorocyclopropyl]-3-methyl-1H-pyrazol- |
| | 4-yl}-5-(difluoromethoxy)-[2,3′-bipyridin]-6′-yl)-3-[(1R*)-1-(5- |
| | methylpyrimidin-2-yl)ethyl]urea (Isomer 2) |
| I-398 | (1S,2R)-2-[5-(difluoromethyl)pyrimidin-2-yl]-N-{6-[3-methyl-1- |
| | (2,2,2-trifluoroethyl)-1H-pyrazol-4-yl]-5-[(2R*)-oxetan-2-yl]- |
| | [2,3′-bipyridin]-6′-yl}cyclopropane-1-carboxamide (Isomer 1) |
| I-399 | (1S,2R)-2-[5-(difluoromethyl)pyrimidin-2-yl]-N-{6-[3-methyl-1- |
| | (2,2,2-trifluoroethyl)-1H-pyrazol-4-yl]-5-[(2R*)-oxetan-2-yl]- |
| | [2,3′-bipyridin]-6′-yl}cyclopropane-1-carboxamide (Isomer 2) |
| I-400 | (1S,2R)-2-[5-(difluoromethyl)pyrimidin-2-yl]-N-(6-{1-[(1R,2S)- |
| | 2-fluorocyclopropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-401 | (1S,2R)-2-[5-(difluoromethyl)pyrimidin-2-yl]-N-(6-{1-[(1R,2S)- |
| | 2-fluorocyclopropyl]-5-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-402 | (1S,2R)-2-[5-(difluoromethyl)pyrimidin-2-yl]-N-(6-{1-[(1R,2R)- |
| | 2-fluorocyclopropyl]-3-methyl-1H-pyrazol-4-yl}-5-[(1S)-1- |
| | hydroxyethyl]-[2,3′-bipyridin]-6′-yl)cyclopropane-1-carboxamide |
| | (Isomer 1) |
| I-403 | (1S,2R)-2-[5-(difluoromethyl)pyrimidin-2-yl]-N-(6-{1-[(1R,2R)- |
| | 2-fluorocyclopropyl]-5-methyl-1H-pyrazol-4-yl}-5-[(1S)-1- |
| | hydroxyethyl]-[2,3′-bipyridin]-6′-yl)cyclopropane-1-carboxamide |
| | (Isomer 2) |
| I-404 | (1S,2R)-2-[5-(difluoromethyl)pyridin-2-yl]-N-{5-[(1S)-1- |
| | hydroxyethyl]-4′,6-dimethoxy-[2,3′-bipyridin]-6′- |
| | yl}cyclopropane-1-carboxamide |
| I-405 | (1S,2R)-N-{4′-[(2,2-difluoroethyl)amino]-5-[(1S)-1- |
| | hydroxyethyl]-6-methoxy-[2,3′-bipyridin]-6′-yl}-2-[5- |
| | (difluoromethyl)pyridin-2-yl]cyclopropane-1-carboxamide |
| I-406 | (1S,2R)-N-[4′-(cyclobutylamino)-5-[(1S)-1-hydroxyethyl]-6- |
| | methoxy-[2,3′-bipyridin]-6′-yl]-2-[5-(difluoromethyl)pyridin-2- |
| | yl]cyclopropane-1-carboxamide |
| I-407 | (1S,2R)-2-[5-(difluoromethyl)pyridin-2-yl]-N-[4′-(ethylamino)-5- |
| | [(1S)-1-hydroxyethyl]-6-methoxy-[2,3′-bipyridin]-6′- |
| | yl]cyclopropane-1-carboxamide |
| I-409 | (1S,2R)-2-[5-(difluoromethyl)pyrimidin-2-yl]-N-{5-[(1S)-1- |
| | hydroxyethyl]-6-[3-(methylsulfanyl)-1-(2,2,2-trifluoroethyl)-1H- |
| | pyrazol-4-yl]-[2,3′-bipyridin]-6′-yl}cyclopropane-1-carboxamide |
| I-410 | (1S,2R)-2-(5-cyclobutylpyrimidin-2-yl)-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-411 | (1S,2R)-2-{5-[(dimethylamino)methyl]pyrimidin-2-yl}-N-{5- |
| | [(1S)-1-hydroxyethyl]-6-[3-methyl-1-(2,2,2-trifluoroethyl)-1H- |
| | pyrazol-4-yl]-[2,3′-bipyridin]-6′-yl}cyclopropane-1-carboxamide |
| I-412 | (1S,2R)-N-{5-[(1S)-1-hydroxyethyl]-6-[5-methyl-1-(2,2,2- |
| | trifluoroethyl)-1H,4H,5H,6H,7H-pyrazolo[4,3-c]pyridin-3-yl]- |
| | [2,3′-bipyridin]-6′-yl}-2-(5-methylpyrimidin-2-yl)cyclopropane- |
| | 1-carboxamide |
| I-413 | (1S,2R)-2-[4-(fluoromethyl)-5-methylpyrimidin-2-yl]-N-{5- |
| | [(1S)-1-hydroxyethyl]-6-[3-methyl-1-(2,2,2-trifluoroethyl)-1H- |
| | pyrazol-4-yl]-[2,3′-bipyridin]-6′-yl}cyclopropane-1-carboxamide |
| I-414 | (1S,2R)-2-[5-(difluoromethyl)-4-methylpyrimidin-2-yl]-N-{5- |
| | [(1S)-1-hydroxyethyl]-6-[3-methyl-1-(2,2,2-trifluoroethyl)-1H- |
| | pyrazol-4-yl]-[2,3′-bipyridin]-6′-yl}cyclopropane-1-carboxamide |
| I-415 | 3-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-1-[(1R*)-1-(5- |
| | methylpyridin-2-yl)ethyl]urea (Isomer 2) |
| I-416 | (1S,2R)-N-{6-[1-(2,2-difluoroethyl)-3-methyl-1H-pyrazol-4-yl]- |
| | 5-[(1S)-1-hydroxyethyl]-[2,3′-bipyridin]-6′-yl}-2-[5- |
| | (difluoromethyl)pyrimidin-2-yl]cyclopropane-1-carboxamide |
| I-417 | (1S,2R)-2-[5-(difluoromethyl)pyrimidin-2-yl]-N-{5-[(1S)-1- |
| | hydroxyethyl]-6-[5-methyl-1-(2,2,2-trifluoroethyl)-1H-pyrazol-4- |
| | yl]-[2,3′-bipyridin]-6′-yl}cyclopropane-1-carboxamide |
| I-418 | (1S,2R)-2-[5-(difluoromethyl)pyrimidin-2-yl]-N-{6-[1-(2- |
| | fluoroethyl)-3-methyl-1H-pyrazol-4-yl]-5-[(1S)-1-hydroxyethyl]- |
| | [2,3′-bipyridin]-6′-yl}cyclopropane-1-carboxamide |
| I-419 | (1S,2R)-N-{6-[1-(2,2-difluoroethyl)-3-methyl-1H-pyrazol-4-yl]- |
| | 5-[(1S)-1-hydroxyethyl]-[2,3′-bipyridin]-6′-yl}-2-[5- |
| | (difluoromethyl)pyridin-2-yl]cyclopropane-1-carboxamide |
| I-420 | (1S,2R)-2-[5-(difluoromethyl)pyridin-2-yl]-N-{6-[1-(2- |
| | fluoroethyl)-3-methyl-1H-pyrazol-4-yl]-5-[(1S)-1-hydroxyethyl]- |
| | [2,3′-bipyridin]-6′-yl}cyclopropane-1-carboxamide |
| I-421 | (1S,2R)-2-(5-cyclopropylpyrimidin-2-yl)-N-{6-[1-(2- |
| | fluoroethyl)-3-methyl-1H-pyrazol-4-yl]-5-[(1S)-1-hydroxyethyl]- |
| | [2,3′-bipyridin]-6′-yl}cyclopropane-1-carboxamide |
| I-422 | (1S,2R)-N-{6-[5-cyano-3-(difluoromethyl)-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide (Isomer 1) |
| I-423 | (1S,2R)-N-{5-cyano-6-[(1S,2R)-2-(dimethylamino)cyclobutoxy]- |
| | [2,3′-bipyridin]-6′-yl}-2-(5-methylpyridin-2-yl)cyclopropane-1- |
| | carboxamide |
| I-424 | (1S,2R)-N-{5-[(1S)-1-hydroxyethyl]-6-[6-methyl-1-(2,2,2- |
| | trifluoroethyl)-1H,4H,5H,6H,7H-pyrazolo[3,4-c]pyridin-3-yl]- |
| | [2,3′-bipyridin]-6′-yl}-2-(5-methylpyrimidin-2-yl)cyclopropane- |
| | 1-carboxamide |
| I-425 | (1S,2R)-N-{6-[5-cyano-3-(difluoromethyl)-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide (Isomer 2) |
| I-426 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5- |
| | (oxetan-3-yl)pyrimidin-2-yl]cyclopropane-1-carboxamide |
| I-427 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5-(1- |
| | methylazetidin-3-yl)pyrimidin-2-yl]cyclopropane-1-carboxamide |
| I-428 | (1S,2R)-2-[5-(difluoromethyl)pyridin-2-yl]-N-{5-[(1S)-1- |
| | hydroxyethyl]-6-[4-methyl-1-(2,2,2-trifluoroethyl)-1H-pyrazol-3- |
| | yl]-[2,3′-bipyridin]-6′-yl}cyclopropane-1-carboxamide |
| I-429 | (1S,2R)-2-[5-(difluoromethyl)pyridin-2-yl]-N-{5-[(1S)-1- |
| | hydroxyethyl]-6-[4-methyl-1-(2,2,2-trifluoroethyl)-1H-pyrazol-5- |
| | yl]-[2,3′-bipyridin]-6′-yl}cyclopropane-1-carboxamide |
| I-430 | (1S,2R)-N-{6-[3-cyano-5-(difluoromethyl)-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide (Isomer 1) |
| I-431 | (1S,2R)-N-{6-[3-cyano-5-(difluoromethyl)-1H-pyrazol-1-yl]-5- |
| | [(1R*)-1-hydroxyethyl]-[2,3′-bipyridin]-6′-yl}-2-(5- |
| | methylpyridin-2-yl)cyclopropane-1-carboxamide (Isomer 2) |
| I-432 | (1S,2R)-N-(6-{1-[(2,2-difluorocyclopropyl)methyl]-3-methyl- |
| | 1H-pyrazol-4-yl}-5-[(1S)-1-hydroxyethyl]-[2,3′-bipyridin]-6′-yl)- |
| | 2-[5-(difluoromethyl)pyridin-2-yl]cyclopropane-1-carboxamide |
| I-433 | (1S,2R)-2-[5-(difluoromethyl)pyridin-2-yl]-N-(6-{ 1-[(1- |
| | fluorocyclopropyl)methyl]-3-methyl-1H-pyrazol-4-yl}-5-[(1S)-1- |
| | hydroxyethyl]-[2,3′-bipyridin]-6′-yl)cyclopropane-1-carboxamide |
| I-434 | (1S,2R)-N-(6-{1-[(1-fluorocyclopropyl)methyl]-3-methyl-1H- |
| | pyrazol-4-yl}-5-[(1S)-1-hydroxyethyl]-[2,3′-bipyridin]-6′-yl)-2- |
| | (5-methylpyrimidin-2-yl)cyclopropane-1-carboxamide |
| I-435 | (1S,2R)-N-[6-(1-{[(1R*)-2,2-difluorocyclopropyl]methyl}-3- |
| | methyl-1H-pyrazol-4-yl)-5-[(1S)-1-hydroxyethyl]-[2,3′- |
| | bipyridin]-6′-yl]-2-[5-(difluoromethyl)pyridin-2-yl]cyclopropane- |
| | 1-carboxamide (Isomer 1) |
| I-436 | (1S,2R)-N-[6-(1-{[(1R*)-2,2-difluorocyclopropyl]methyl}-3- |
| | methyl-1H-pyrazol-4-yl)-5-[(1S)-1-hydroxyethyl]-[2,3′- |
| | bipyridin]-6′-yl]-2-[5-(difluoromethyl)pyridin-2-yl]cyclopropane- |
| | 1-carboxamide (Isomer 2) |
| I-437 | (1S,2R)-N-[6-(1-{[(1R*)-2,2-difluorocyclopropyl]methyl}-5- |
| | methyl-1H-pyrazol-4-yl)-5-[(1S)-1-hydroxyethyl]-[2,3′- |
| | bipyridin]-6′-yl]-2-[5-(difluoromethyl)pyridin-2-yl]cyclopropane- |
| | 1-carboxamide (Isomer 1) |
| I-438 | (1S,2R)-N-[6-(1-{[(1R*)-2,2-difluorocyclopropyl]methyl}-5- |
| | methyl-1H-pyrazol-4-yl)-5-[(1S)-1-hydroxyethyl]-[2,3′- |
| | bipyridin]-6′-yl]-2-[5-(difluoromethyl)pyridin-2-yl]cyclopropane- |
| | 1-carboxamide (Isomer 2) |
| I-439 | (1S,2R)-2-[5-(difluoromethyl)pyrimidin-2-yl]-N-(6-{1-[(1- |
| | fluorocyclopropyl)methyl]-3-methyl-1H-pyrazol-4-yl}-5-[(1S)-1- |
| | hydroxyethyl]-[2,3′-bipyridin]-6′-yl)cyclopropane-1-carboxamide |
| I-440 | (1S,2R)-2-[5-(difluoromethyl)pyridin-2-yl]-N-(6-{1-[(1- |
| | fluorocyclopropyl)methyl]-5-methyl-1H-pyrazol-4-yl}-5-[(1S)-1- |
| | hydroxyethyl]-[2,3′-bipyridin]-6′-yl)cyclopropane-1-carboxamide |
| I-441 | (1S,2R)-N-(6-{1-[(1-fluorocyclopropyl)methyl]-5-methyl-1H- |
| | pyrazol-4-yl}-5-[(1S)-1-hydroxyethyl]-[2,3′-bipyridin]-6′-yl)-2- |
| | (5-methylpyrimidin-2-yl)cyclopropane-1-carboxamide |
| I-442 | (1S,2R)-2-[5-(difluoromethyl)pyrimidin-2-yl]-N-(6-{1-[(1- |
| | fluorocyclopropyl)methyl]-5-methyl-1H-pyrazol-4-yl}-5-[(1S)-1- |
| | hydroxyethyl]-[2,3′-bipyridin]-6′-yl)cyclopropane-1-carboxamide |
| I-443 | (1S,2R)-N-{5-[(1R*)-1-hydroxypropyl]-6-[3-methyl-1-(2,2,2- |
| | trifluoroethyl)-1H-pyrazol-4-yl]-[2,3′-bipyridin]-6′-yl}-2-(5- |
| | methylpyrimidin-2-yl)cyclopropane-1-carboxamide (Isomer 1) |
| I-444 | (1S,2R)-2-[5-(difluoromethyl)pyridin-2-yl]-N-{5-[(1R*)-1- |
| | hydroxy-2-methylpropyl]-6-[3-methyl-1-(2,2,2-trifluoroethyl)- |
| | 1H-pyrazol-4-yl]-[2,3′-bipyridin]-6′-yl}cyclopropane-1- |
| | carboxamide (Isomer 1) |
| I-445 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 1) |
| I-446 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 2) |
| I-447 | (1S,2R)-2-[5-(difluoromethyl)pyridin-2-yl]-N-{5-[(1S)-1- |
| | hydroxyethyl]-6-[1-(2,2,2-trifluoroethyl)-1H-pyrazol-4-yl]-[2,3′- |
| | bipyridin]-6′-yl}cyclopropane-1-carboxamide |
| I-448 | (1S,2R)-N-{5-[(1S)-1-hydroxyethyl]-6-{[(1R*,4R**,5S*)-2- |
| | methyl-2-azabicyclo[3.1.0]hexan-4-yl]oxy}-[2,3′-bipyridin]-6′- |
| | yl}-2-(5-methylpyridin-2-yl)cyclopropane-1-carboxamide |
| | (Isomer 1) |
| I-449 | (1S,2R)-N-{5-[(1S)-1-hydroxyethyl]-6-{[(1R*,4R**,5S*)-2- |
| | methyl-2-azabicyclo[3.1.0]hexan-4-yl]oxy}-[2,3′-bipyridin]-6′- |
| | yl}-2-(5-methylpyridin-2-yl)cyclopropane-1-carboxamide |
| | (Isomer 2) |
| I-450 | (1S,2R)-N-{5-[(1S)-1-hydroxyethyl]-6-{[(1R*,4R**,5S*)-2- |
| | methyl-2-azabicyclo[3.1.0]hexan-4-yl]oxy}-[2,3′-bipyridin]-6′- |
| | yl}-2-(5-methylpyridin-2-yl)cyclopropane-1-carboxamide |
| | (Isomer 3) |
| I-451 | (1S,2R)-N-{5-[(1S)-1-hydroxyethyl]-6-{[(1R*,4R**,5S*)-2- |
| | methyl-2-azabicyclo[3.1.0]hexan-4-yl]oxy}-[2,3′-bipyridin]-6′- |
| | yl}-2-(5-methylpyridin-2-yl)cyclopropane-1-carboxamide |
| | (Isomer 4) |
| I-452 | (1S,2R)-2-[5-(difluoromethyl)pyridin-2-yl]-N-{5-[(1R*)-1- |
| | hydroxy-2-methylpropyl]-6-[3-methyl-1-(2,2,2-trifluoroethyl)- |
| | 1H-pyrazol-4-yl]-[2,3′-bipyridin]-6′-yl}cyclopropane-1- |
| | carboxamide (Isomer 2) |
| I-453 | (1S,2R)-N-{5-[(1R*)-1-hydroxypropyl]-6-[3-methyl-1-(2,2,2- |
| | trifluoroethyl)-1H-pyrazol-4-yl]-[2,3′-bipyridin]-6′-yl}-2-(5- |
| | methylpyrimidin-2-yl)cyclopropane-1-carboxamide (Isomer 2) |
| I-454 | (1S,2R)-N-(6-{1-[(1R*)-2,2-difluorocyclopropyl]-5-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 1) |
| I-455 | (1S,2R)-N-(6-{1-[(1R*)-2,2-difluorocyclopropyl]-5-methyl-1H- |
| | pyrazol-4-yl}-5-[(1S)-1-hydroxyethyl]-[2,3′-bipyridin]-6′-yl)-2- |
| | [5-(difluoromethyl)pyridin-2-yl]cyclopropane-1-carboxamide |
| | (Isomer 2) |
| I-456 | (1S,2R)-2-[5-(difluoromethyl)pyridin-2-yl]-N-{5-[(1R*)-1- |
| | hydroxypropyl]-6-[3-methyl-1-(2,2,2-trifluoroethyl)-1H-pyrazol- |
| | 4-yl]-[2,3′-bipyridin]-6′-yl}cyclopropane-1-carboxamide (Isomer 1) |
| I-457 | (1S,2R)-2-[5-(difluoromethyl)pyridin-2-yl]-N-{5-[(1R*)-1- |
| | hydroxypropyl]-6-[3-methyl-1-(2,2,2-trifluoroethyl)-1H-pyrazol- |
| | 4-yl]-[2,3′-bipyridin]-6′-yl}cyclopropane-1-carboxamide (Isomer 2) |
| I-458 | (1S,2R)-N-(6-{1-azaspiro[3.3]heptan-6-yloxy}-5-cyano-[2,3′- |
| | bipyridin]-6′-yl)-2-(5-methylpyridin-2-yl)cyclopropane-1- |
| | carboxamide |
| I-459 | (1S,2R)-N-{5-cyano-6-[(1S,2R)-2-(methylamino)cyclobutoxy]- |
| | [2,3′-bipyridin]-6′-yl}-N-methyl-2-(5-methylpyridin-2- |
| | yl)cyclopropane-1-carboxamide |
| I-460 | (1S,2R)-2-[5-(difluoromethyl)pyrimidin-2-yl]-N-{6-[1-(3,3- |
| | difluoropropyl)-3-methyl-1H-pyrazol-4-yl]-5-[(1S)-1- |
| | hydroxyethyl]-[2,3′-bipyridin]-6′-yl} cyclopropane-1- |
| | carboxamide |
| I-461 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (5-methylpyrimidin-2-yl)cyclopropane-1-carboxamide (Isomer 1) |
| I-462 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (5-methylpyrimidin-2-yl)cyclopropane-1-carboxamide (Isomer 2) |
| I-463 | 3-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-1-[(1R*)-1-(5- |
| | methylpyrimidin-2-yl)ethyl]urea (Isomer 1) |
| I-464 | 3-{5-[(1S)-1-hydroxyethyl]-6-[3-methyl-1-(2,2,2-trifluoroethyl)- |
| | 1H-pyrazol-4-yl]-[2,3′-bipyridin]-6′-yl}-1-[(1R*)-1-(5- |
| | methylpyrimidin-2-yl)ethyl]urea (Isomer 2) |
| I-465 | (1S,2R)-2-(5-{bicyclo[1.1.1]pentan-1-yl}pyrimidin-2-yl)-N-{6- |
| | [1-(2,2-difluoroethyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-466 | (1S,2R)-N-{6-[3-(2,2-difluoroethyl)-5-methyl-1H-pyrazol-1-yl]- |
| | 5-[(1S)-1-hydroxyethyl]-[2,3′-bipyridin]-6′-yl}-2-[5- |
| | (difluoromethyl)pyrimidin-2-yl]cyclopropane-1-carboxamide |
| I-467 | (1S,2R)-2-[5-(1,1-difluoroethyl)pyrimidin-2-yl]-N-{5-[(1S)-1- |
| | hydroxyethyl]-6-[3-methyl-1-(2,2,2-trifluoroethyl)-1H-pyrazol-4- |
| | yl]-[2,3′-bipyridin]-6′-yl}cyclopropane-1-carboxamide |
| I-468 | (1S,2R)-2-[5-(difluoromethyl)pyrimidin-2-yl]-N-{5-[(1S)-1- |
| | hydroxyethyl]-6-[3-methyl-1-(oxetan-3-yl)-1H-pyrazol-4-yl]- |
| | [2,3′-bipyridin]-6′-yl}cyclopropane-1-carboxamide |
| I-469 | (1S,2R)-2-[5-(difluoromethyl)pyrimidin-2-yl]-N-{5-[(1S)-1- |
| | hydroxyethyl]-6-[5-methyl-3-(2,2,2-trifluoroethyl)-1H-pyrazol-1- |
| | yl]-[2,3′-bipyridin]-6′-yl}cyclopropane-1-carboxamide |
| I-470 | (1S,2R)-N-{5-[(1R*)-2,2-difluoro-1-hydroxyethyl]-6-[3-methyl- |
| | 1-(2,2,2-trifluoroethyl)-1H-pyrazol-4-yl]-[2,3′-bipyridin]-6′-yl}- |
| | 2-[5-(difluoromethyl)pyrimidin-2-yl]cyclopropane-1- |
| | carboxamide (Isomer 1) |
| I-471 | (1S,2R)-N-{5-[(1R*)-2,2-difluoro-1-hydroxyethyl]-6-[3-methyl- |
| | 1-(2,2,2-trifluoroethyl)-1H-pyrazol-4-yl]-[2,3′-bipyridin]-6′-yl}- |
| | 2-[5-(difluoromethyl)pyrimidin-2-yl]cyclopropane-1- |
| | carboxamide (Isomer 2) |
| I-472 | (3R*)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-3-(5- |
| | methylpyridin-2-yl)butanamide (Isomer 1) |
| I-473 | (3R*)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-3-(5- |
| | methylpyridin-2-yl)butanamide (Isomer 2) |
| I-474 | 3-[6-(3-cyano-5-methyl-1H-pyrazol-1-yl)-5-[(1S)-1- |
| | hydroxyethyl]-[2,3′-bipyridin]-6′-yl]-1-[(1R)-1-[5- |
| | (difluoromethyl)pyridin-2-yl]ethyl]urea |
| I-475 | (1S,2R)-2-[5-(difluoromethyl)pyrimidin-2-yl]-N-{6-[3-ethyl-1- |
| | (2,2,2-trifluoroethyl)-1H-pyrazol-4-yl]-5-[(1S)-1-hydroxyethyl]- |
| | [2,3′-bipyridin]-6′-yl}cyclopropane-1-carboxamide |
| I-476 | (1S,2R)-2-[5-(3-fluoroazetidin-1-yl)pyrimidin-2-yl]-N-{5-[(1S)- |
| | 1-hydroxyethyl]-6-[3-methyl-1-(2,2,2-trifluoroethyl)-1H-pyrazol- |
| | 4-yl]-[2,3′-bipyridin]-6′-yl}cyclopropane-1-carboxamide |
| I-477 | (1S,2R)-2-[5-(difluoromethyl)pyrimidin-2-yl]-N-{6-[1-(2,2- |
| | difluoropropyl)-3-methyl-1H-pyrazol-4-yl]-5-[(1S)-1- |
| | hydroxyethyl]-[2,3′-bipyridin]-6′-yl}cyclopropane-1- |
| | carboxamide |
| I-478 | (1S,2R)-N-{6-[1-(3,3-difluorocyclobutyl)-3-methyl-1H-pyrazol- |
| | 4-yl]-5-[(1S)-1-hydroxyethyl]-[2,3′-bipyridin]-6′-yl}-2-[5- |
| | (difluoromethyl)pyrimidin-2-yl]cyclopropane-1-carboxamide |
| I-479 | (1S,2R)-N-[6-(3-cyano-5-methyl-1H-pyrazol-1-yl)-5-[(1S)-1- |
| | hydroxyethyl]-[2,3′-bipyridin]-6′-yl]-2-[5-(3,3-difluoroazetidin-1- |
| | yl)pyrimidin-2-yl]cyclopropane-1-carboxamide |
| I-480 | (1S,2R)-N-(6-{1-[(1R*)-2,2-difluorocyclopropyl]-3-methyl-1H- |
| | pyrazol-4-yl}-5-[(1S)-1-hydroxyethyl]-[2,3′-bipyridin]-6′-yl)-2- |
| | (5-{2-oxabicyclo[2.1.1]hexan-4-yl}pyridin-2-yl)cyclopropane-1- |
| | carboxamide (Isomer 2) |
| I-481 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-[5-(2- |
| | hydroxypropan-2-yl)pyridin-2-yl]cyclopropane-1-carboxamide |
| I-482 | (1S,2R)-2-{5-[(1R,5S)-3-azabicyclo[3.1.0]hexan-3-yl]pyrimidin- |
| | 2-yl}-N-{5-[(1S)-1-hydroxyethyl]-6-[3-methyl-1-(2,2,2- |
| | trifluoroethyl)-1H-pyrazol-4-yl]-[2,3′-bipyridin]-6′- |
| | yl}cyclopropane-1-carboxamide |
| I-483 | (1S,2R)-2-[5-(difluoromethyl)pyridin-2-yl]-N-{5-[(1S)-1- |
| | hydroxyethyl]-4′-(methylamino)-6-(methylsulfanyl)-[2,3′- |
| | bipyridin]-6′-yl}cyclopropane-1-carboxamide |
| I-484 | (1S,2R)-N-{6-[3-(difluoromethyl)-1-(2,2,2-trifluoroethyl)-1H- |
| | pyrazol-4-yl]-5-[(1S)-1-hydroxyethyl]-[2,3′-bipyridin]-6′-yl}-2- |
| | (5-methylpyrimidin-2-yl)cyclopropane-1-carboxamide |
| I-485 | (1S,2R)-N-{5-[(1S)-1-hydroxyethyl]-6-[3-methyl-1-(2,2,2- |
| | trifluoroethyl)-1H-pyrazol-4-yl]-[2,3′-bipyridin]-6′-yl}-2-{5- |
| | [(2R)-2-methylazetidin-1-yl]pyrimidin-2-yl}cyclopropane-1- |
| | carboxamide |
| I-486 | (1S,2R)-N-[6-(1-{[(1S)-2,2-difluorocyclopropyl]methyl}-3- |
| | methyl-1H-pyrazol-4-yl)-5-[(1S)-1-hydroxyethyl]-[2,3′- |
| | bipyridin]-6′-yl]-2-[5-(difluoromethyl)pyrimidin-2- |
| | yl]cyclopropane-1-carboxamide |
| I-487 | (1S,2R)-2-[5-(difluoromethyl)pyrimidin-2-yl]-N-{5-[(1S)-1- |
| | hydroxyethyl]-6-{3-methyl-1-[(oxetan-3-yl)methyl]-1H-pyrazol- |
| | 4-yl}-[2,3′-bipyridin]-6′-yl}cyclopropane-1-carboxamide |
| I-488 | (1S,2R)-2-{5-[1-(difluoromethyl)cyclopropyl]pyrimidin-2-yl}-N- |
| | {5-[(1S)-1-hydroxyethyl]-6-[3-methyl-1-(2,2,2-trifluoroethyl)- |
| | 1H-pyrazol-4-yl]-[2,3′-bipyridin]-6′-yl}cyclopropane-1- |
| | carboxamide |
| I-489 | (1S,2R)-2-[5-(3,3-difluoroazetidin-1-yl)pyrimidin-2-yl]-N-{5- |
| | [(1S)-1-hydroxyethyl]-6-[3-methyl-1-(2,2,2-trifluoroethyl)-1H- |
| | pyrazol-4-yl]-[2,3′-bipyridin]-6′-yl}cyclopropane-1-carboxamide |
| I-490 | 3-{5-[(1S)-1-hydroxyethyl]-6-[5-methyl-2-(2,2,2-trifluoroethyl)- |
| | 2H-1,2,3-triazol-4-yl]-[2,3′-bipyridin]-6′-yl}-1-[(1R*)-1-(5- |
| | methylpyrimidin-2-yl)ethyl]urea (Isomer 1) |
| I-491 | 3-{5-[(1S)-1-hydroxyethyl]-6-[5-methyl-2-(2,2,2-trifluoroethyl)- |
| | 2H-1,2,3-triazol-4-yl]-[2,3′-bipyridin]-6′-yl}-1-[(1R*)-1-(5- |
| | methylpyrimidin-2-yl)ethyl]urea (Isomer 2) |
| I-492 | (1S,2R)-2-[5-(difluoromethyl)pyridin-2-yl]-N-(6-{3- |
| | [(dimethylamino)methyl]-1-(2,2,2-trifluoroethyl)-1H-pyrazol-4- |
| | yl}-5-[(1S)-1-hydroxyethyl]-[2,3′-bipyridin]-6′-yl)cyclopropane- |
| | 1-carboxamide |
| I-493 | (1S,2R)-2-[5-(3,3-difluoroazetidin-1-yl)pyridin-2-yl]-N-(6-{1- |
| | [(1R*)-2,2-difluorocyclopropyl]-3-methyl-1H-pyrazol-4-yl}-5- |
| | [(1S)-1-hydroxyethyl]-[2,3′-bipyridin]-6′-yl)cyclopropane-1- |
| | carboxamide (Isomer 2) |
| I-494 | (1S,2R)-N-[6-(3-cyano-4,5-dimethyl-1H-pyrazol-1-yl)-5-[(1S)-1- |
| | hydroxyethyl]-[2,3′-bipyridin]-6′-yl]-2-[5- |
| | (difluoromethyl)pyrimidin-2-yl]cyclopropane-1-carboxamide |

In some embodiments, the present disclosure provides a compound set forth in Table 1, above, or a pharmaceutically acceptable salt thereof. In some embodiments, the present disclosure provides a compound set forth in Table 1, above.

In some embodiments, the present disclosure provides a pharmaceutical composition comprising a compound disclosed herein (described in embodiments herein, both singly and in combination), or a pharmaceutically acceptable salt thereof, together with a pharmaceutically acceptable carrier, excipient, or diluent. For example, in some embodiments, the present disclosure provides a pharmaceutical composition comprising a compound of formula I as defined above, or a pharmaceutically acceptable salt thereof, together with a pharmaceutically acceptable carrier, excipient, or diluent. In some embodiments, the present disclosure provides a pharmaceutical composition comprising a compound of formula I as defined above, together with a pharmaceutically acceptable carrier, excipient, or diluent. In some embodiments, the present disclosure provides a pharmaceutical composition comprising a compound set forth in Table 1 above, or a pharmaceutically acceptable salt thereof, together with a pharmaceutically acceptable carrier, excipient, or diluent. In some embodiments, the present disclosure provides a pharmaceutical composition comprising a compound set forth in Table 1 above, together with a pharmaceutically acceptable carrier, excipient, or diluent.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. In certain embodiments, the amount of compound in compositions of this disclosure is such that is effective to measurably inhibit SIK2, or a mutant thereof, in a biological sample or in a patient. In certain embodiments, a composition of this disclosure is formulated for administration to a patient in need of such composition. In some embodiments, a composition of this disclosure is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitory active metabolite or residue thereof.

As used herein, the term “active metabolite or residue thereof” means that a metabolite or residue thereof is also an inhibitor of SIK2, or a mutant thereof.

The subject matter disclosed herein includes prodrugs, metabolites, derivatives, and pharmaceutically acceptable salts of compounds of the disclosure. Metabolites include compounds produced by a process comprising contacting a compound of the disclosure with a mammal for a period of time sufficient to yield a metabolic product thereof. If the compound of the disclosure is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the disclosure is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

A compound of the disclosure can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the disclosure are prodrugs of any of the formulae herein.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

The amount of compounds of the present disclosure that may be combined with the carrier materials to produce a composition in a single dosage form will vary depending upon the host treated, the particular mode of administration. Preferably, provided compositions should be formulated so that a dosage of between 0.01 and 100 mg/kg, 0.01 and 50 mg/kg, or 1 and 25 mg/kg, body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

Compounds of the disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment.

### 4. Uses of Compounds and Pharmaceutically Acceptable Compositions

Compounds and compositions described herein are generally useful for the inhibition of kinase activity of one or more enzymes. In some embodiments the kinase inhibited by the compounds and methods of the disclosure is SIK2

The presently disclosed compounds find use in inhibiting SIK2 protein kinase. In one embodiment, the subject matter disclosed herein is directed to a method of inhibiting SIK2, the method comprising contacting SIK2 with an effective amount of a compound of the disclosure or a pharmaceutical composition described herein.

The presently disclosed compounds can be used in a method for inhibiting SIK2. Such methods comprise contacting SIK2 with an effective amount of a presently disclosed compound.

By “contact” is intended bringing the compound within close enough proximity to an isolated SIK2 kinase or a cell expressing SIK2 such that the compound is able to bind to and inhibit the SIK2.

The compound can be contacted with SIK2 in vitro or in vivo via administration of the compound to a subject.

In one aspect, provided herein is a method of inhibiting SIK2 in a biological sample. The method comprises contacting the sample with a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Any method known in the art to measure the kinase activity of SIK2 may be used to determine if SIK2 has been inhibited, including in vitro assays, immunoblots with antibodies specific for phosphorylated targets of SIK2, or the measurement of a downstream biological effect of SIK2 kinase activity.

Provided herein are compounds and pharmaceutical compositions that inhibit SIK2 protein kinase, as well as methods of treatment using such compounds and pharmaceutical compositions. The compounds and compositions can be used in methods of modulating the immune system, for treatment of diseases, and for treatment of cells in vivo, in vitro, or ex vivo.

The present disclosure provides methods of inhibiting SIK2 in a patient. The method comprises administering to a patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

The presently disclosed compounds can be used to treat a SIK2-mediated disorder. As used herein, a “SIK2-mediated disorder” is a pathological condition in which SIK2 activity is necessary for the genesis or maintenance of the pathological condition.

Accordingly, in one aspect, provided herein is a method of treating a SIK2-mediated disorder, disease, or condition in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). In some embodiments, the present disclosure provided a compound disclosed herein (such as a compound of formula I), a pharmaceutically acceptable salt thereof, or a pharmaceutical composition disclosed herein for use in medicine.

In some embodiments, the SIK2-mediated disorder is selected from inflammatory diseases, autoinflammatory diseases, autoimmune diseases, proliferative diseases, fibrotic diseases, transplantation rejection, diseases involving impairment of cartilage turnover, congenital cartilage malformation, diseases involving impairment of bone turnover, diseases associated with hypersecretion of TNFα, interferons, IL-6, IL-12 and/or IL-23, respiratory diseases, endocrine and/or metabolic diseases, cardiovascular diseases, dermatological diseases, and abnormal angiogenesis associated diseases.

In some embodiments, the present disclosure provides a method of treating an inflammatory disease in a patient in need thereof, wherein the method comprises administering to the patient a compound disclosed herein (such as a compound of formula I) or a pharmaceutically acceptable salt thereof.

In some embodiments, the present disclosure provides a method of treating an autoinflammatory disease in a patient in need thereof, wherein the method comprises administering to the patient a compound disclosed herein (such as a compound of formula I) or a pharmaceutically acceptable salt thereof.

In some embodiments, the present disclosure provides a method of treating an autoimmune disease in a patient in need thereof, wherein the method comprises administering to the patient a compound disclosed herein (such as a compound of formula I) or a pharmaceutically acceptable salt thereof.

In some embodiments, the present disclosure provides a method of treating cancer in a patient in need thereof, wherein the method comprises administering to the patient a compound disclosed herein (such as a compound of formula I) or a pharmaceutically acceptable salt thereof. In some embodiments, the cancer is selected from solid tumors (e.g., bone cancer, skin cancer, prostate cancer, colon cancer, esophageal cancer, endometrial cancer, ovarian cancer, uterine cancer, renal cancer, kidney cancer, bladder cancer, hepatic cancer, pancreatic cancer, gastric cancer, breast cancer, lung cancer, cancers of the head and neck, thyroid cancer, glioblastoma, sarcoma, bile duct cancer, bladder cancer), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, the SIK2-mediated disorder is a cancer selected from ovarian cancer, breast cancer, acute myeloid leukemia, and multiple myeloma.

In some embodiments, the disease or disorder is cancer. In some embodiments, the cancer is a solid tumor cancer. In some embodiments, the cancer is lung cancer, non-small cell lung cancer (NSCLC), bone cancer, pancreatic cancer, skin cancer, cancer of the head and neck, cutaneous or intraocular melanoma, uterine cancer, ovarian cancer, colorectal cancer, anal cancer, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer, hormone-refractory prostate cancer, bladder cancer, kidney cancer, renal cell carcinoma, carcinoma of the renal pelvis, pediatric malignancy, neoplasms of the central nervous system, primary CNS lymphoma, spinal axis tumors, medulloblastoma, brain stem gliomas, or pituitary adenomas. In some embodiments, the cancer is a liquid tumor cancer. In some embodiments, the cancer is a leukemia. In some embodiments, the leukemia is acute lymphocytic leukemia (ALL), acute myelogenous leukemia (AML), chronic lymphocytic leukemia (CLL), or chronic myelogenous leukemia (CML). In some embodiments, the cancer is a lymphoma. In some embodiments, the lymphoma is small lymphocytic lymphoma (SLL), cutaneous B-cell lymphoma, cutaneous T-cell lymphoma, Hodgkin's lymphoma, non-Hodgkin's lymphoma, or Waldenstrom macroglobulinemia.

In yet another embodiment, provided herein are methods for treating diseases or disorders selected from the group consisting of inflammatory diseases, autoinflammatory diseases, autoimmune diseases, proliferative diseases, fibrotic diseases, transplantation rejection, diseases involving impairment of cartilage turnover, congenital cartilage malformation, diseases involving impairment of bone turnover, diseases associated with hypersecretion of IL-6, diseases associated with hypersecretion of TNFα, interferons, IL-12 and/or IL-23, respiratory diseases, endocrine and/or metabolic diseases, cardiovascular diseases, dermatological diseases, and abnormal angiogenesis associated diseases, in a subject in need thereof, the method comprising administering a therapeutically effective amount of a compound described herein, or a pharmaceutically acceptable salt, or stereoisomer thereof, or a pharmaceutical composition described herein.

In some embodiments, the disease or disorder is an inflammatory disease. In some embodiments, the inflammatory disease is rheumatoid arthritis, osteoarthritis, allergic airway disease (e.g., asthma), chronic obstructive pulmonary disease (COPD), endometriosis, or inflammatory bowel diseases (e.g., Crohn's disease, ulcerative colitis).

In some embodiments, the disease or disorder is an autoinflammatory disease. In some embodiments, the autoinflammatory disease is Cryopyrin-Associated Periodic Syndromes (CAPS), Familial Mediterranean Fever (FMF), tumor necrosis factor receptor-associated periodic syndrome (TRAPS), Behcet's disease, Systemic-onset Juvenile Idiopathic Arthritis (SJIA), Still's disease, hemophagocytic lymphohistiocytosis, or macrophage activation syndrome.

In other embodiments, the present disclosure also provides methods for improving, restoring or enhancing fertility and for increasing oocyte production in a female subject by administering to the female subject an effective amount of a compound described herein, or a pharmaceutically acceptable salt, or stereoisomer thereof, or a pharmaceutical composition described herein.

In some embodiments, the disease or disorder is an autoimmune disease. In some embodiments, the autoimmune disease is COPD, asthma, bronchitis, systemic lupus erythematosus (SLE), cutaneous lupus erythematosus (CLE), lupus nephritis, dermatomyositis, autoimmune hepatitis, primary sclerosing cholangitis, primary biliary cirrhosis, Sjogren's syndrome, multiple sclerosis, psoriasis, dry eye disease, type I diabetes mellitus, atopic dermatitis, thyroiditis, contact dermatitis, eczematous dermatitis, inflammatory bowel disease (e.g., Crohn's disease and ulcerative colitis), atherosclerosis, or amyotrophic lateral sclerosis.

In some embodiments, the disease or disorder is a proliferative disease. In some embodiments, the proliferative disease is cancer, myeloproliferative disorders, leukemia, multiple myeloma, psoriasis, restenosis, scleroderma, or fibrosis.

In some embodiments, the disease or disorder is a fibrotic disease. In some embodiments, the fibrotic disease is idiopathic pulmonary fibrosis (IPF), Dupuytren's disease, nonalcoholic steatohepatitis (NASH), systemic sclerosis, renal fibrosis, cutaneous fibrosis or fibrostenotic Crohn's disease.

In some embodiments, the disease or disorder involves impairment of bone turnover. In some embodiments, the term diseases involving impairment of bone turnover refers to osteoporosis, osteopenia, hormone deficiency, hormone excess, Paget's disease, osteoarthritis, renal bone disease, osteogenesis imperfecta, or hypophosphatasia.

In some embodiments, the disease or disorder is associated with hypersecretion of IL-6. In some embodiments, the disease associated with hypersecretion of IL-6 is Castleman's disease, multiple myeloma, psoriasis, Kaposi's sarcoma, or mesangial proliferative glomerulonephritis

In some embodiments, the disease or disorder is associated with hypersecretion of TNFα, interferons, IL-12 and/or IL-23. In some embodiments, the disease associated with hypersecretion of TNFα, interferons, IL-12 and/or IL-23 is systemic and cutaneous lupus erythematosus, lupus nephritis, dermatomyositis, Sjogren's syndrome, psoriasis, rheumatoid arthritis, polymyalgia rheumatica, giant cell arteritis psoriatic arthritis, multiple sclerosis, trisomy 21, ulcerative colitis, or Crohn's disease.

In some embodiments, the disease or disorder is an inflammatory disease, an autoinflammatory disease, an autoimmune disease, a proliferative disease, a fibrotic disease, transplantation rejection, a disease involving impairment of cartilage turnover, congenital cartilage malformation, a diseases involving impairment of bone turnover, a disease associated with hypersecretion of IL-6, a disease associated with hypersecretion of TNFα, interferons, IL-12 and/or IL-23, a respiratory disease, an endocrine and/or metabolic disease, a cardiovascular disease, a dermatological disease, or an abnormal angiogenesis associated disease.

In other aspects, the present disclosure also provides methods for treating conditions or disorders including: Arthritis, including rheumatoid arthritis, juvenile arthritis, and psoriatic arthritis;

Autoimmune or inflammatory diseases or disorders, including Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, autoimmune hepatitis, primary sclerosing cholangitis, chronic aggressive hepatitis, non-alcoholic fatty liver disease, non-alcoholic steatohepatitis ulcerative colitis and membranous glomerulopathy, systemic lupus erythematosus, rheumatoid arthritis, psoriatic arthritis, Sjogren's syndrome, Reiter's syndrome, polymyositis, dermatomyositis, type I interferonopathies including Aicardi-Goutieres syndrome and other mendelian diseases of overexpression of type I interferon systemic sclerosis, polyarteritis nodosa, multiple sclerosis, relapsing remitting multiple sclerosis, primary progressive multiple sclerosis, secondary progressive multiple sclerosis, and bullous pemphigoid, and additional autoimmune diseases, which can be O-cell (humoral) based or T-cell based, including Cogan's syndrome, ankylosing spondylitis, Wegener's granulomatosis, autoimmune alopecia, Type I or juvenile onset diabetes, gout, thyroiditis or bacterial sepsis;

Diabetes, including Type I diabetes or complications from diabetes;

Eye diseases, disorders or conditions including autoimmune diseases of the eye, keratoconjunctivitis, vernal conjunctivitis, uveitis including non-infectious uveitis associated with Behcet's disease, non-infectious uveitis, and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, or ocular neovascularization;

Intestinal inflammations, including Crohn's disease, ulcerative colitis, pouchitis, inflammatory bowel disease, celiac disease, proctitis, eosinophilic gastroenteritis, mastocytosis, gastrointestinal mucositis, oral mucositis or pouchitis;

Neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemia, renal ischemia, heart attacks, cardiac hypertrophy, atherosclerosis and arteriosclerosis, organ hypoxia, or platelet aggregation; Skin diseases, conditions or disorders including atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummular dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, Vogt-Koyanagi-Harada disease, Sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, hidradenitis suppurativa, psoriasis, plaque psoriasis, pustular psoriasis, nail psoriasis, flexural psoriasis, guttate psoriasis, psoriatic arthritis, erythrodermic psoriasis, or inverse psoriasis;

Allergic reactions including allergic dermatitis in mammal (including horse allergic diseases such as bite hypersensitivity), summer eczema, sweet itch in horses, heaves, inflammatory airway disease, recurrent airway obstruction, airway hyper-responsiveness, or chronic obstruction pulmonary disease;

Asthma and other obstructive airways diseases, including chronic or inveterate asthma, late asthma, bronchitis, bronchial asthma, allergic asthma, intrinsic asthma, extrinsic asthma, or dust asthma; and

Transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In some embodiments, the present disclosure provides a method of treating ovarian cancer in a patient in need thereof, wherein the method comprises administering to the patient a compound disclosed herein (such as a compound of formula I) or a pharmaceutically acceptable salt thereof.

In some embodiments, the present disclosure provides a method of treating breast cancer in a patient in need thereof, wherein the method comprises administering to the patient a compound disclosed herein (such as a compound of formula I) or a pharmaceutically acceptable salt thereof.

In some embodiments, the present disclosure provides a method of treating acute myeloid leukemia in a patient in need thereof, wherein the method comprises administering to the patient a compound disclosed herein (such as a compound of formula I) or a pharmaceutically acceptable salt thereof.

In some embodiments, the present disclosure provides a method of treating multiple myeloma in a patient in need thereof, wherein the method comprises administering to the patient a compound disclosed herein (such as a compound of formula I) or a pharmaceutically acceptable salt thereof.

In some embodiments, the present disclosure provides a method of treating fibrotic diseases in a patient in need thereof, wherein the method comprises administering to the patient a compound disclosed herein (such as a compound of formula I) or a pharmaceutically acceptable salt thereof.

In some embodiments, the present disclosure provides a method of treating transplantation rejection diseases involving impairment of cartilage turnover in a patient in need thereof, wherein the method comprises administering to the patient a compound disclosed herein (such as a compound of formula I) or a pharmaceutically acceptable salt thereof.

In some embodiments, the present disclosure provides a method of treating congenital cartilage malformation in a patient in need thereof, wherein the method comprises administering to the patient a compound disclosed herein (such as a compound of formula I) or a pharmaceutically acceptable salt thereof.

In some embodiments, the present disclosure provides a method of treating diseases involving impairment of bone turnover in a patient in need thereof, wherein the method comprises administering to the patient a compound disclosed herein (such as a compound of formula I) or a pharmaceutically acceptable salt thereof.

In some embodiments, the present disclosure provides a method of treating diseases associated with hypersecretion of TNFα, interferons, IL-6, IL-12 and/or IL-23 in a patient in need thereof, wherein the method comprises administering to the patient a compound disclosed herein (such as a compound of formula I) or a pharmaceutically acceptable salt thereof.

In some embodiments, the present disclosure provides a method of treating respiratory diseases in a patient in need thereof, wherein the method comprises administering to the patient a compound disclosed herein (such as a compound of formula I) or a pharmaceutically acceptable salt thereof.

In some embodiments, the present disclosure provides a method of treating endocrine and/or metabolic diseases in a patient in need thereof, wherein the method comprises administering to the patient a compound disclosed herein (such as a compound of formula I) or a pharmaceutically acceptable salt thereof.

In some embodiments, the present disclosure provides a method of treating cardiovascular diseases, including pulmonary arterial hypertension or complications from pulmonary arterial hypertension in a patient in need thereof, wherein the method comprises administering to the patient a compound disclosed herein (such as a compound of formula I) or a pharmaceutically acceptable salt thereof.

In some embodiments, the present disclosure provides a method of treating dermatological diseases in a patient in need thereof, wherein the method comprises administering to the patient a compound disclosed herein (such as a compound of formula I) or a pharmaceutically acceptable salt thereof.

In some embodiments, the present disclosure provides a method of treating abnormal angiogenesis associated diseases in a patient in need thereof, wherein the method comprises administering to the patient a compound disclosed herein (such as a compound of formula I) or a pharmaceutically acceptable salt thereof.

In some embodiments, the SIK2-mediated disorder is inflammatory bowel diseases such as Crohn's disease and ulcerative colitis, diabetes, a skin pigmentation disease, osteoporosis, osteoarthritis, lupus (systemic lupus erythematosus), a musculoskeletal disease, or rheumatoid arthritis.

In some embodiments, the SIK2-mediated disorder is Bone-mineral disorders, including chronic kidney disease. In some embodiments, the SIK2-mediated disorder is a mineral bone disorder. In some embodiments, the SIK2-mediated disorder is a mineral loss disorder, such as Malignancy-related and oncology drug-related bone loss, and osteoporosis (post-menopausal, glucocorticoid-induced, hypogonadal).

In some embodiments, the SIK2-mediated disorder is Ankylosing Spondylitis, Psoriatic Arthritis, Crohn's Disease (Active and Maintenance), Plaque Psoriasis, Juvenile Idiopathic Arthritis, Ulcerative Colitis, Hidradenitis Suppurativa, Uveitis, and non-radiographic axial spondyloarthritis.

In some embodiments, the present disclosure provides a method of treating inflammatory bowel disease in a patient in need thereof, wherein the method comprises administering to the patient a compound disclosed herein (such as a compound of formula I) or a pharmaceutically acceptable salt thereof.

In some embodiments, the present disclosure provides a method of treating diabetes in a patient in need thereof, wherein the method comprises administering to the patient a compound disclosed herein (such as a compound of formula I) or a pharmaceutically acceptable salt thereof.

In some embodiments, the present disclosure provides a method of treating a skin pigmentation disease in a patient in need thereof, wherein the method comprises administering to the patient a compound disclosed herein (such as a compound of formula I) or a pharmaceutically acceptable salt thereof.

In some embodiments, the present disclosure provides a method of treating osteoporosis in a patient in need thereof, wherein the method comprises administering to the patient a compound disclosed herein (such as a compound of formula I) or a pharmaceutically acceptable salt thereof.

In some embodiments, the present disclosure provides a method of treating osteoarthritis in a patient in need thereof, wherein the method comprises administering to the patient a compound disclosed herein (such as a compound of formula I) or a pharmaceutically acceptable salt thereof.

In some embodiments, the present disclosure provides a method of treating a musculoskeletal disease in a patient in need thereof, wherein the method comprises administering to the patient a compound disclosed herein (such as a compound of formula I) or a pharmaceutically acceptable salt thereof.

In some embodiments, the present disclosure provides a method of treating rheumatoid arthritis in a patient in need thereof, wherein the method comprises administering to the patient a compound disclosed herein (such as a compound of formula I) or a pharmaceutically acceptable salt thereof.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the disclosure or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the SIK2 inhibitor is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

In some embodiments, the SIK2 inhibitor is administered to the subject at a dose of between about 0.1 mg and about 10 g, including but not limited to about 1 mg, 5 mg, 10 mg, 25 mg, 50 mg, 100 mg, 200 mg, 250 mg, 500 mg, 750 mg, 1 g, 2 g, 5 g, and 10 g.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments, the compounds of the disclosure are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Additionally, provided are SIK2 inhibitors for use as therapeutic active substances. A SIK2 inhibitor for use in treating or preventing a SIK2-mediated disorder provided herein. Further provided is the use of a SIK2 inhibitor in the manufacture of a medicament for treating or preventing a SIK2-mediated disorder provided herein.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

### 5. Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.” In certain embodiments, a provided combination, or composition thereof, is administered in combination with another therapeutic agent.

Those additional agents may be administered separately from a provided combination therapy, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another.

As used herein, the term “combination,” “combined,” and related terms refers to the simultaneous or sequential administration of therapeutic agents in accordance with this disclosure.

For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present disclosure provides a composition comprising a compound of formula I and one or more additional therapeutic agents. The therapeutic agent may be administered together with a compound of formula I, or may be administered prior to or following administration of a compound of formula I. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can besides or in addition be administered especially for tumor therapy in combination with chemotherapy, radiotherapy, immunotherapy, phototherapy, surgical intervention, or a combination thereof. Long-term therapy is equally possible as is adjuvant therapy in the context of other treatment strategies, as described above.

Other possible treatments are therapy to maintain the patient's status after tumor regression, or even chemopreventive therapy, for example in patients at risk.

### EXAMPLES

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein. Additional compounds of the disclosure were prepared by methods substantially similar to those described herein in the Examples and methods known to one skilled in the art.

### List of Abbreviations

| | |
|---|---|
| Ac: acetyl | cataCXium ® A Pd G3: |
| AcOH: acetic acid | methanesulfonato[(di(1-adamantyl)-n- |
| ACN: acetonitrile | butylphos-phine)-2-(2′-amino-1,1′- |
| AlMe₃: trimethylaluminium | biphenyl)]palladium |
| Aq: aqueous | CDI: carbonyldiimidazole |
| B₂Pin₂: bis (pinacolato)diboron- | CH₂Cl₂: dichloromethane |
| 4,4,4′,4′,5,5,5′,5′-octamethyl-2,2′-bi(1,3,2- | CD₃OD: deuterated methanol |
| dioxaborolane) | CDCl₃: deuterated chloroform |
| Bn: benzyl | Cs₂CO₃: cesium carbonate |
| Boc: tert-butoxycarbonyl | d: days |
| Boc₂O: di-tert-butyl dicarbonate | DAST: diethylaminosulfur trifluoride |
| cataCXium: di-adamantylalkylphosphine | dba: dibenzylideneacetone |
| DCM: dichloromethane | DCE: 1,2-dichloroethane |
| DEA: diethylamine | HATU: N,N,N′,N′-tetramethyl-O-(7- |
| DIBAL-H: diisobutylaluminum hydride | azabenzotriazol-1-yl)uronium |
| DIPA: diisopropylamine | hexafluorophosphate |
| DIPEA or DIEA: N,N- | HCl: hydrochloric acid |
| diisopropylethylamine | HPLC: high performance liquid |
| DMA: N,N-dimethylacetamide | chromatography |
| DME: 1,2-dimethoxyethane | HOAc: acetic acid |
| DMAP: 4-dimethylaminopyridine | iPrOH: isopropyl alcohol |
| DMF: N,N-dimethylformamide | IPA: isopropyl alcohol |
| DMP: Dess-Martin periodinane | KOAc: potassium acetates |
| DMSO: dimethyl sulfoxide | K₂CO₃: potassium carbonate |
| DMSO-d6: deuterated dimethyl sulfoxide | K₂PO₄: potassium phosphate |
| DPPA: diphenylphosphoryl azide | LDA: lithium diisopropylamide |
| CataCXium A: bis (1-adamantyl)-butyl- | LIHMDS: lithium hexamethyldisilazide |
| phosphane | M: molar |
| dppf: 1,1′- | MeCN: acetonitrile |
| bis(diphenylphosphino)ferrocene | MeOH: methanol |
| EDC or EDCI: 1-(3- | MeSNa: sodium thiomethoxide |
| dimethylaminopropyl)-3- | min: minutes |
| ethylcarbodiimide hydrochloride | mL: milliliters |
| ee: enantiomeric excess | mM: millimolar |
| ESI: electrospray ionization | mmol: millimoles |
| EA: ethyl acetate | MPa: mega pascal |
| EtOAc: ethyl acetate | MTBE: methyl tert-butyl ether |
| EtOH: ethanol | NH₄OH: ammonium hydroxide |
| Et₃N•3HF: triethylamine trihydrofluoride | N₂: nitrogen |
| FA: formic acid | NAD: Nicotinamide adenine dinucleotide |
| GDH: glutamate dehydrogenase | NADP: Nicotinamide adenine |
| h or hrs: hours | dinucleotide phosphate |
| NaBH₄: sodium borohydride | NaOH: sodium hydroxide |
| NH₄HCO₃: ammonium bicarbonate | Na₂SO₄: sodium sulfate |
| NH₄Cl: ammonium chloride | PBS: phosphate buffered saline |
| NBS: N-bromosuccinimide | PE: petroleum ether |
| NiCl₂(dme): | PPh₃: triphenylphosphine |
| dichloro(dimethoxyethane)nickel | Rel: relative |
| NMP: N-methylpyrrolidine | RT or rt: room temperature |
| NMR: nuclear magnetic resonance | Rt: retention time |
| ° C.: degrees Celsius | sat: saturated |
| Cy₃P-Pd-G3: [(tricyclohexylphosphine)- | SFC: supercritical fluid chromatography |
| 2-(2′-aminobiphenyl)]palladium(II) | SOCl₂: sulfur dichloride |
| methanesulfonate | TBAB: tetrabutylammonium bromide |
| Pd/C: palladium on carbon | TBAI: tetrabutylammonium iodide |
| Pd(dba)₂: palladium | TEA: triethylamine |
| bis(dibenzylideneacetone) | TfOH: trifluoromethanesulfonic acid |
| Pd₂(dba)₃: tris(dibenzylideneacetone) | TFAA: trifluoroacetic acid anhydride |
| dipalladium dipalladium | TMS: trimethylsilyl |
| Pd(dppf)Cl₂•CH₂Cl₂: [1,1′- | TFA: trifluoracetic acid |
| Bis(diphenylphosphino)ferrocene]dichlor | THF: tetrahydrofuran |
| opalladium(II), complex with | TLC: thin layer chromatography |
| dichloromethane | wt: weight |
| PCy3-Pd-G3: | XPhos Pd G2: chloro(2- |
| methanesulfonato(tricyclohexylphosphine) | dicyclohexylphosphino-2′,4′,6′- |
| (2′-amino-1,1′-biphenyl-2- | triisopropyl-1,1′-biphenyl)[2-(2′-amino- |
| yl)palladium(II) | 1,1′-biphenyl)]palladium(II) |
| Pd(OAc)₂: palladium acetate | Xphos Pd G4: (SP-4-3)- |
| Sccm-standard cubic centimeters per | [Dicyclohexy][2′,4′,6′-tris(1- |
| minute | methylethyl)[1,1′-biphenyl]-2- |
| Pd(PPh₃)₄: palladium | yl]phosphine](methanesulfonato-κO)[2′- |
| tetrakis(triphenylphosphine) | (methylamino-κN)[1,1′-biphenyl]-2-yl- |
| | κC]palladium |

### General Synthetic Methods

All starting materials, building blocks, reagents, acids, bases, dehydrating agents, solvents, and catalysts utilized to synthesis the compounds of the present disclosure were either commercially available or can be produced by organic synthesis methods known to one of ordinary skill in the art (Houben-Weyl 4th Ed. 1952, Methods of Organic Synthesis, Thieme, Volume 21). Further, the compounds of the present disclosure can be produced by organic synthesis methods known to one of ordinary skill in the art as shown in the following examples.

Proton NMR (¹H NMR) was conducted in deuterated solvent. In certain compounds disclosed herein, one or more ¹H shifts overlap with residual protic solvent signals; these signals have not been reported in the experimental provided hereinafter.

LCMS was conduct using the following methods:

| | |
|---|---|
| Instrument | Shimadzu LCMS-2020 |
| Stationary Phase | Kinetex PS C18 3.0 × 2.1 mm, 2.6 μm |
| Mode | Binary Gradient |
| Mobile Phase A | 0.1% FA in water (v/v) |
| Mobile Phase B | 0.07% FA in Acetonitrile (v/v) |
| Gradient | 5% to 100% B in 2.29 min, hold 100% B in 0.5 |
| | min |
| Column Dimensions | 3.0 × 2.1 mm, 2.6 μm |
| Column Temperature | 40° C. |
| Flow Rate | 1.2 mL/min |

### LCMS 2

| | |
|---|---|
| Instrument | Shimadzu LCMS-2020 |
| Stationary Phase | Shim-pack Scepter 33 × 3.0 mm, 3 μm |
| Mode | Binary Gradient |
| Mobile Phase A | 5 mM NH₄HCO₃ in H₂O/Acetonitrile (95:5, V/V) |
| Mobile Phase B | Acetonitrile |
| Gradient | 20% to 90% B in 2.29 min, hold 90% B in 0.5 |
| | min |
| Column Dimensions | 33 × 3.0 mm, 3 μm |
| Column Temperature | 40° C. |
| Flow Rate | 1.5 mL/min |

### LCMS 3

| | |
|---|---|
| Instrument | Shimadzu LCMS-2020 |
| Stationary Phase | Halo C18, 30 × 3.0 mm, 2.0 um |
| Mode | Binary Gradient |
| Mobile Phase A | water/0.5% TFA |
| Mobile Phase B | Acetonitrile/0.5% TFA |
| Gradient | 5% to 100% B in 1.19 min, hold 100% B in 0.6 |
| | min |
| Column Dimensions | 30 × 3.0 mm, 2.0 um |
| Column Temperature | 40° C. |
| Flow Rate | 1.5 mL/min |

### LCMS 4

| | |
|---|---|
| Instrument | Shimadzu LCMS-2020 |
| Stationary Phase | Halo C18, 3.0 × 30 mm, 5.0 um |
| Mode | Binary Gradient |
| Mobile Phase A | water/0.0375% TFA |
| Mobile Phase B | Acetonitrile/0.01875% TFA |
| Gradient | 5% to 95% B in 0.5 min, hold 95% B in 0.3 min, |
| | 95% to 5% B in 0.25 min. |
| Column Dimensions | 3.0 × 30 mm, 5.0 um |
| Column Temperature | 50° C. |
| Flow Rate | 1.5 mL/min |

### LCMS 5

| | |
|---|---|
| Instrument | Shimadzu LCMS-2020 |
| Stationary Phase | Halo C18, 3.0 × 30 mm, 5.0 um |
| Mode | Binary Gradient |
| Mobile Phase A | 0.025% NH₄OH in water (v/v) |
| Mobile Phase B | Acetonitrile |
| Gradient | 5% to 95% B in 0.8 min, hold 95% B in 0.4 min, |
| | 95% to 5% B in 0.35 min. |
| Column Dimensions | 30 × 3.0 mm, 2.0 um |
| Column Temperature | 40° C. |
| Flow Rate | 1.5 mL/min |

### LCMS 6

| | |
|---|---|
| Instrument | Shimadzu LCMS-2020 |
| Mode | Binary Gradient |
| Stationary Phase | Shim-pack Scepter C18 |
| Mobile Phase A | 5 mM NH₄HCO₃ in H₂O/Acetonitrile(95:5, V/V) |
| Mobile Phase B | Acetonitrile |
| Column Dimensions | 33 × 3.0 mm, 3.0 um |
| Flow Rate | 1.0 mL/min |
| Column Temperature | 40° C. |
| Gradient | 30% to 90% B in 2.29 min, hold 90% B in 0.5 |
| | min |

### LCMS 7

| | |
|---|---|
| Instrument | ANL-MCL5-LCMS-034 |
| Mode | Binary Gradient |
| Stationary Phase | Acquity BEH C18 |
| Mobile Phase A | Water/5 mM FA |
| Mobile Phase B | ACN/5 mM FA |
| Column Dimensions | 50 mm × 2.1 mm, 1.7 um |
| Flow Rate | 0.6 mL/min |
| Column Temperature | 35° C. |
| Gradient | 3% to 98% B in 5 min, hold 98% B in 2 min |

### General Synthetic Methods of Producing Compounds of the Disclosure

Compounds of the present disclosure can generally be prepared by conventional techniques known to those skilled in the art or by processes analogous to those described in the accompanying synthetic schemes.

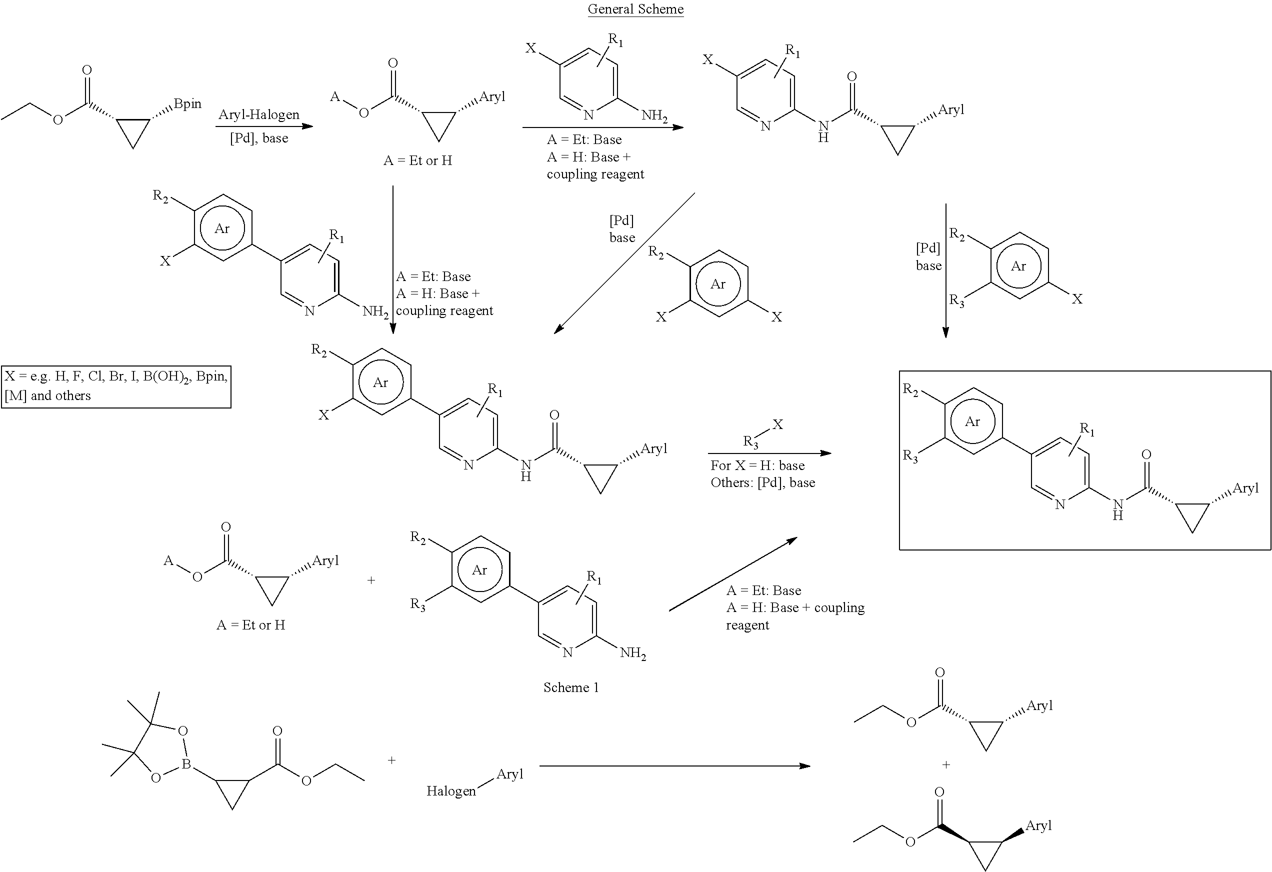

### EXAMPLES OF THE DISCLOSURE

### Example 1. Synthesis of (1S,2R)—N-{5-[(1S)-1-hydroxyethyl]-6-[3-methyl-1-(2,2,2-trifluoroethyl)-1H-pyrazol-4-yl]-[2,3′-bipyridin]-6′-yl}-2-(5-methylpyridin-2-yl)cyclopropane-1-carboxamide (I-6) and (1S,2R)—N-{5-[(1R)-1-hydroxyethyl]-6-[3-methyl-1-(2,2,2-trifluoroethyl)-1H-pyrazol-4-yl]-[2,3′-bipyridin]-6′-yl}-2-(5-methylpyridin-2-yl)cyclopropane-1-carboxamide (I-135)

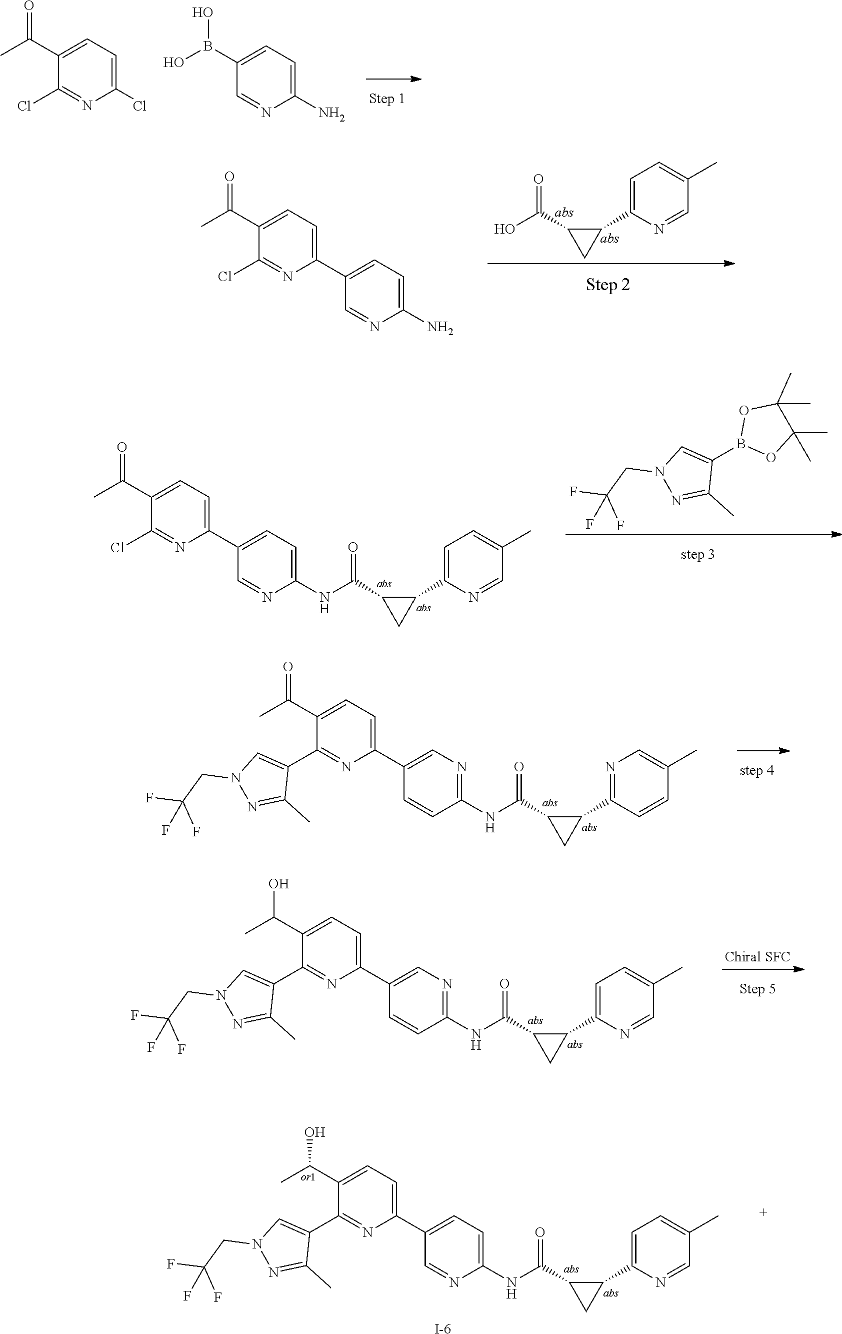

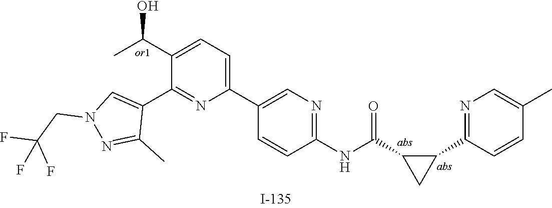 [SMILES: Cc1ccc([C@@H]2C[C@@H]2C(=O)Nc2ccc(-c3ccc([C@@H](C)O)c(-c4cn(CC(F)(F)F)nc4C)n3)cn2)nc1]

Step 1. Synthesis of 1-(6′-amino-6-chloro-[2,3′-bipyridin]-5-yl)ethan-1-one. A mixture of (6-amino-3-pyridyl)boronic acid (10.00 g, 72.50 mmol, 1 eq), 1-(2,6-dichloro-3-pyridyl)ethanone (20.67 g, 108.75 mmol, 1.5 eq), Pd(dppf)Cl₂·CH₂Cl₂ (5.92 g, 7.25 mmol, 0.1 eq), K₃PO₄ (38.47 g, 181.25 mmol, 2.5 eq) in dioxane (200 mL) and H₂O (40 mL) was stirred at 40° C. for 2 h under N₂ atmosphere. The reaction mixture was concentrated under reduced pressure to give a residue. The residue was purified by flash silica gel chromatography (Eluent of PE/EtOAc) to afford the title compound. ¹H NMR (400 MHz, DMSO-d6) δ (ppm): 8.73 (1H), 8.17 (1H), 8.12-8.08 (1H), 7.94 (1H), 6.58 (2H), 6.54 (1H), 2.62 (3H). LCMS: 248.2 [M+H]⁺.

Step 2. Synthesis of (1S,2R)—N-(5-acetyl-6-chloro-[2,3′-bipyridin]-6′-yl)-2-(5-methylpyridin-2-yl)cyclopropane-1-carboxamide. To a solution of (1S,2R)-2-(5-methyl-2-pyridyl)cyclopropanecarboxylic acid (950 mg, 5.36 mmol, 1 eq) and 1-(6′-amino-6-chloro-[2,3′-bipyridin]-5-yl)ethan-1-one (1.39 g, 5.63 mmol, 1.05 eq) in pyridine (10 mL) was added HATU (3.06 g, 8.04 mmol, 1.5 eq), then the mixture was stirred at 80° C. for 1 h. After cooling down to room temperature the mixture was poured into H₂O (100 mL) and filtered. The filter cake was dried to give a residue. The residue was purified by flash silica gel chromatography (Eluent of PE/EtOAc, then EtOAc/MeOH) to afford the title compound. ¹H NMR (400 MHz, DMSO-d6) δ (ppm): 11.06 (1H), 9.02 (1H), 8.39-8.34 (1H), 8.28-8.24 (2H), 8.13 (1H), 7.95 (1H), 7.53-7.48 (1H), 7.24 (1H), 2.68 (1H), 2.64 (3H), 2.48 (1H), 2.23 (3H), 1.77-1.71 (1H), 1.46-1.38 (1H). LCMS: 407.2 [M+H]⁺.

Step 3. Synthesis of (1S,2R)—N-(5-acetyl-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide. A mixture of (1S,2R)—N-(5-acetyl-6-chloro-[2,3′-bipyridin]-6′-yl)-2-(5-methylpyridin-2-yl)cyclopropane-1-carboxamide (2.10 g, 5.16 mmol, 1 eq), 3-methyl-4-(4,4,5,5-tetramethyl-1,3,2-dioxaborolan-2-yl)-1-(2,2,2-trifluoroethyl) pyrazole (1.72 g, 5.94 mmol, 1.15 eq), Pd(dppf)Cl₂·CH₂Cl₂ (422 mg, 516 mol, 0.1 eq), K₃PO₄ (2.74 g, 12.90 mmol, 2.5 eq) in dioxane (20 mL) and H₂O (5 mL) was stirred at 80° C. for 2 h under N₂ atmosphere. The mixture was concentrated under reduced pressure to give a residue. The residue was purified by flash silica gel chromatography (Eluent of PE/EtOAc, then EtOAc/MeOH) and to afford the title compound. ¹H NMR (400 MHz, DMSO-d6) δ (ppm): 10.95 (1H), 9.05 (1H), 8.43-8.37 (1H), 8.22 (1H), 8.06-8.02 (1H), 8.00-7.91 (3H), 7.48-7.43 (1H), 7.19 (1H), 5.11 (2H), 2.70-2.61 (1H), 2.49-2.47 (1H), 2.34 (3H), 2.32 (3H), 2.21 (3H), 1.76-1.69 (1H), 1.44-1.35 (1H). LCMS: 535.3 [M+H]⁺.

Step 4. Synthesis of (1S,2R)—N-(5-(1-hydroxyethyl)-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide. A solution of (1S,2R)—N-[5-[5-acetyl-6-[3-methyl-1-(2,2,2-trifluoroethyl) pyrazol-4-yl]-2-pyridyl]-2-pyridyl]-2-(5-methyl-2-pyridyl) cyclopropane carboxamide (3.00 g, 5.61 mmol, 1 eq) in MeOH (60 mL) was added NaBH₄ (425 mg, 11.22 mmol, 2 eq) at 0° C., then the mixture was warmed to 25° C. and stirred at 25° C. for 1 h. The mixture was poured into saturated NH₄Cl (100 mL). Then the mixture was extracted with EtOAc (3×50 mL). Then the organic phase was dried over anhydrous Na₂SO₄ and concentrated under reduced pressure to give a residue. The residue was purified by reverse phase preparative HPLC (MeCN/H₂O, 0.1% FA) to afford the title compound, which was directly subjected to chiral separation (step 5).

Step 5. Synthesis of (1S,2R)—N-(5-((R*)-1-hydroxyethyl)-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide Isomer 1 and Isomer 2. The (1S,2R)—N-(5-(1-hydroxyethyl)-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide was purified by chiral SFC (SFC Preparative Method: Instrument:Waters 150Q Preparative SFC system; Column: Daicel Chiralpak IH column, 250×50 mm I.D., 10 μm particle size; Mobile Phase A: CO₂, Mobile Phase B: IPA(0.1% NH₄OH); Isocratic elution: 45% Phase B in Supercritical CO₂; Flow rate: 150 g/min; Retention time: Peak 1: 4.12 min, Peak 2: 5.21 min; Back Pressure: 80bar to keep the CO₂ in Supercritical flow; Wave Length: 220 nm) to give Peak 1 & Peak 2. Peak 1 was dissolved in MeOH (5 mL) and purified by reverse phase preparative HPLC (MeCN/H₂O, 0.225% formic acid) to afford the title compound I-6. Peak 2 was dissolved in MeOH (5 mL) and purified by reverse phase preparative (MeCN/H₂O, 0.225% formic acid) to afford the title compound I-135.

I-6: (1S,2R)—N-(5-((R*)-1-hydroxyethyl)-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide (Isomer 1). ¹H NMR (400 MHz, CD₃OD) δ (ppm): 8.91 (1H), 8.28-8.23 (2H), 8.11 (1H), 7.93 (1H), 7.89 (1H), 7.83 (1H), 7.60-7.56 (1H), 7.33 (1H), 4.98-4.91 (3H), 2.79-2.68 (1H), 2.52-2.40 (1H), 2.32 (3H), 2.30 (3H), 1.93-1.83 (1H), 1.55-1.47 (1H), 1.41 (3H). LCMS: 537.3.3 [M+H]⁺. Chiral SFC: Rt=1.953 min (instrument: SHIMADZU LC-30ADsf; column: Chiralpak IH-3 50*4.6 mm I.D., particle size 3 mm; mobile phase: CO₂/EtOH (0.05% diethylamine); flow rate 3.0 mL/min, 35° C., 100 bar, 220 nm).

I-135: (1S,2R)—N-(5-((R*)-1-hydroxyethyl)-6-(3-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide (Isomer 2). ¹H NMR (400 MHz, CD₃OD) δ (ppm): 8.90 (1H), 8.28-8.21 (2H), 8.11 (1H), 7.93 (1H), 7.89 (1H), 7.83 (1H), 7.58-7.50 (1H), 7.29 (1H), 4.99-4.91 (3H), 2.77-2.68 (1H), 2.49-2.40 (1H), 2.32 (3H), 2.28 (3H), 1.91-1.84 (1H), 1.55-1.46 (1H), 1.41 (3H). LCMS: 537.3 [M+H]⁺. Chiral SFC: Rt=2.131 min (instrument: SHIMADZU LC-30ADsf; column: Chiralpak IH-3 50×4.6 mm I.D., particle size 3 mm; mobile phase: CO₂/EtOH (0.05% diethylamine); flow rate 3.0 mL/min, 35° C., 100 bar, 220 nm).

### Example 2. Synthesis of (1S,2R)—N-{6-chloro-5-[(1S)-1-hydroxyethyl]-[2,3′-bipyridin]-6′-yl}-2-(5-methylpyridin-2-yl)cyclopropane-1-carboxamide (I-100)

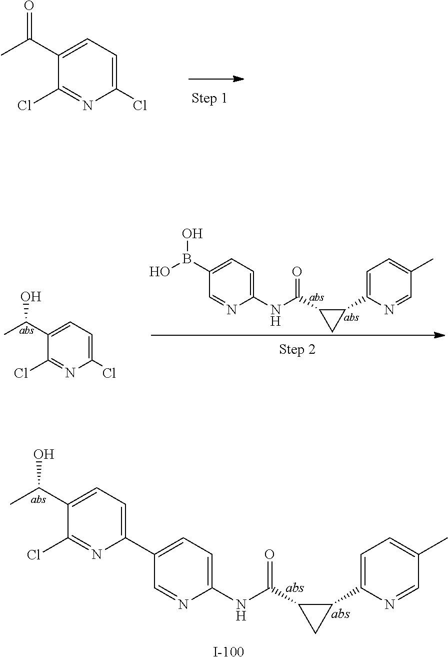 [SMILES: CC(=O)c1ccc(Cl)nc1Cl ; C[C@H](O)c1ccc(Cl)nc1Cl ; Cc1ccc([C@@H]2C[C@@H]2C(=O)Nc2ccc(B(O)O)cn2)nc1 ; Cc1ccc([C@@H]2C[C@@H]2C(=O)Nc2ccc(-c3ccc([C@H](C)O)c(Cl)n3)cn2)nc1]

Step 1. Synthesis of (S)-1-(2,6-dichloropyridin-3-yl)ethan-1-ol. To a solution of 1-(2,6-dichloro-3-pyridyl)ethanone (7.00 g, 36.84 mmol, 1 eq) in DMSO (175 mL) and H₂O (1.75 L) was added tris-buffer (1 M aqueous, 70 mL) and purple carrot cubes (see e.g., Bai, et al, ACS Med. Chem. Lett., 2023, 14, 846-852) (1.40 kg). The mixture was stirred at 25° C. for 48 h under N₂. The mixture was filtered and washed with EtOAc (1 L), the filtrate was extracted with EtOAc (2×1.5 L), the combined organic layers were concentrated to approx. 500 mL, then washed with brine (200 mL×2), dried over anhydrous Na₂SO₄, filtered and concentrated in vacuum to give a residue. The residue was purified by flash silica gel chromatography (Eluent of PE/EtOAc) to afford the title compound. ¹H NMR (400 MHz, DMSO-d6) δ (ppm): 8.04 (1H), 7.60 (1H), 5.64 (1H), 4.99-4.83 (1H), 1.32 (3H). LCMS: 192.1 [M+H]⁺. Chiral SFC: Rt=0.988, ee value=99.54% (instrument: Shimadzu LC-30AD; column: Chiralpak AD-3 50×4.6 mm I.D., particle size 3 m; mobile phase A: CO₂; mobile phase B: MeOH and 0.05% diethylamine; 3.0 mL/min; 35° C.; 10 MPa, 220 nm).

Step 2. Synthesis of (1S,2R)—N-(6-chloro-5-((S)-1-hydroxyethyl)-[2,3′-bipyridin]-6′-yl)-2-(5-methylpyridin-2-yl)cyclopropane-1-carboxamide. To a solution of [6-[[(1S,2R)-2-(5-methyl-2-pyridyl)cyclopropanecarbonyl]amino]-3-pyridyl]boronic acid (100 mg, 337 mol, 1 eq) and (1S)-1-(2,6-dichloro-3-pyridyl)ethanol (95 mg, 495 mol, 1.47 eq) in dioxane (1.5 mL) and water (0.3 mL) was added K₃PO₄ (179 mg, 841 mol, 2.5 eq) and Pd(dppf)Cl₂·CH₂Cl₂ (27 mg, 34 mol, 0.1 eq) at 25° C. under N₂ atmosphere. The mixture was stirred at 40° C. for 2 h. The mixture was cooled to room temperature and then concentrated under reduced pressure to give a residue. The residue was purified by flash silica gel chromatography (Eluent of DCM/MeOH) and reverse phase preparative HPLC (ACN/H₂O, 0.225% FA) to afford the title compound. ¹H NMR (400 MHz, CD₃OD) δ (ppm): 8.93-8.87 (1H), 8.29-8.22 (2H), 8.09-8.02 (1H), 7.91-7.87 (1H), 7.87-7.80 (1H), 7.56 (1H), 7.31 (1H), 5.20-5.10 (1H), 2.78-2.68 (1H), 2.51-2.40 (1H), 2.29 (3H), 1.91-1.85 (1H), 1.54-1.48 (1H), 1.45 (3H). LCMS: 409.2 [M+H]⁺.

### Example 3. Synthesis of (1S,2R)—N-(5-cyano-6-(((S)-1-methylpyrrolidin-3-yl)oxy)-[2,3′-bipyridin]-6′-yl)-2-(5-methylpyridin-2-yl)cyclopropane-1-carboxamide (I-74)

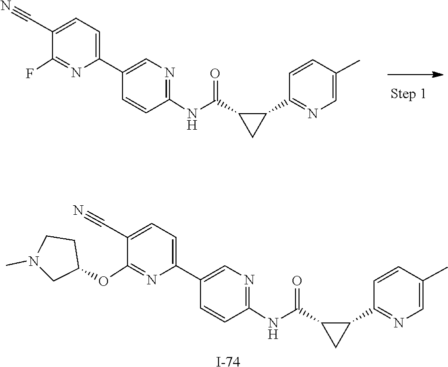

Step 1. To a solution of (3S)-1-methylpyrrolidin-3-ol (8 mg, 80 mol, 1.5 eq) in THF (0.5 mL) was added NaH (5 mg, 134 mol, 60% purity, 2.5 eq) at 0° C. Then (1S,2R)—N-[5-(5-cyano-6-fluoro-2-pyridyl)-2-pyridyl]-2-(5-methyl-2-yridyl)cyclopropanecarboxamide (20 mg, 53.56 mol, 1 eq) was added to the mixture at 0° C. The mixture was stirred at 25° C. for 1 h. The reaction mixture was added dropwise into saturated NH₄Cl (2 mL) and extracted with DCM (3 mL×2). The combined organic layers were washed with brine 10 mL, dried over anhydrous Na₂SO₄, filtered and concentrated under reduced pressure to give a residue. The residue was dissolved in DMF (2 mL) and purified by reverse phase preparative HPLC (ACN/H₂O, 0.05% NH₄OH) to afford the title compound. ¹H NMR (400 MHz, CD₃OD) δ (ppm): 9.01 (1H), 8.40-8.30 (1H), 8.23 (1H), 8.09 (1H), 7.94 (1H), 7.61 (1H), 7.58-7.51 (1H), 7.30 (1H), 5.72-5.62 (1H), 3.09-3.00 (1H), 2.99-2.87 (2H), 2.78-2.68 (1H), 2.63-2.43 (3H), 2.42 (3H), 2.29 (3H), 2.18-2.03 (1H), 1.92-1.83 (1H), 1.55-1.44 (1H). LCMS: 455.3 [M+H]⁺.

### Example 4. Synthesis of ((1S,2R)—N-[5-[6-[1-(2,2-difluoroethyl)-3-methyl-pyrazol-4-yl]-5-[(1S)-1-hydroxyethyl]-2-pyridyl]-2-pyridyl]-2-(5-methyl-2-pyridyl)cyclopropanecarboxamide (I-15) & ((1S,2R)—N-[5-[6-[1-(2,2-difluoroethyl)-5-methyl-pyrazol-4-yl]-5-[(1S)-1-hydroxyethyl]-2-pyridyl]-2-pyridyl]-2-(5-methyl-2-pyridyl)cyclopropanecarboxamide (I-44)

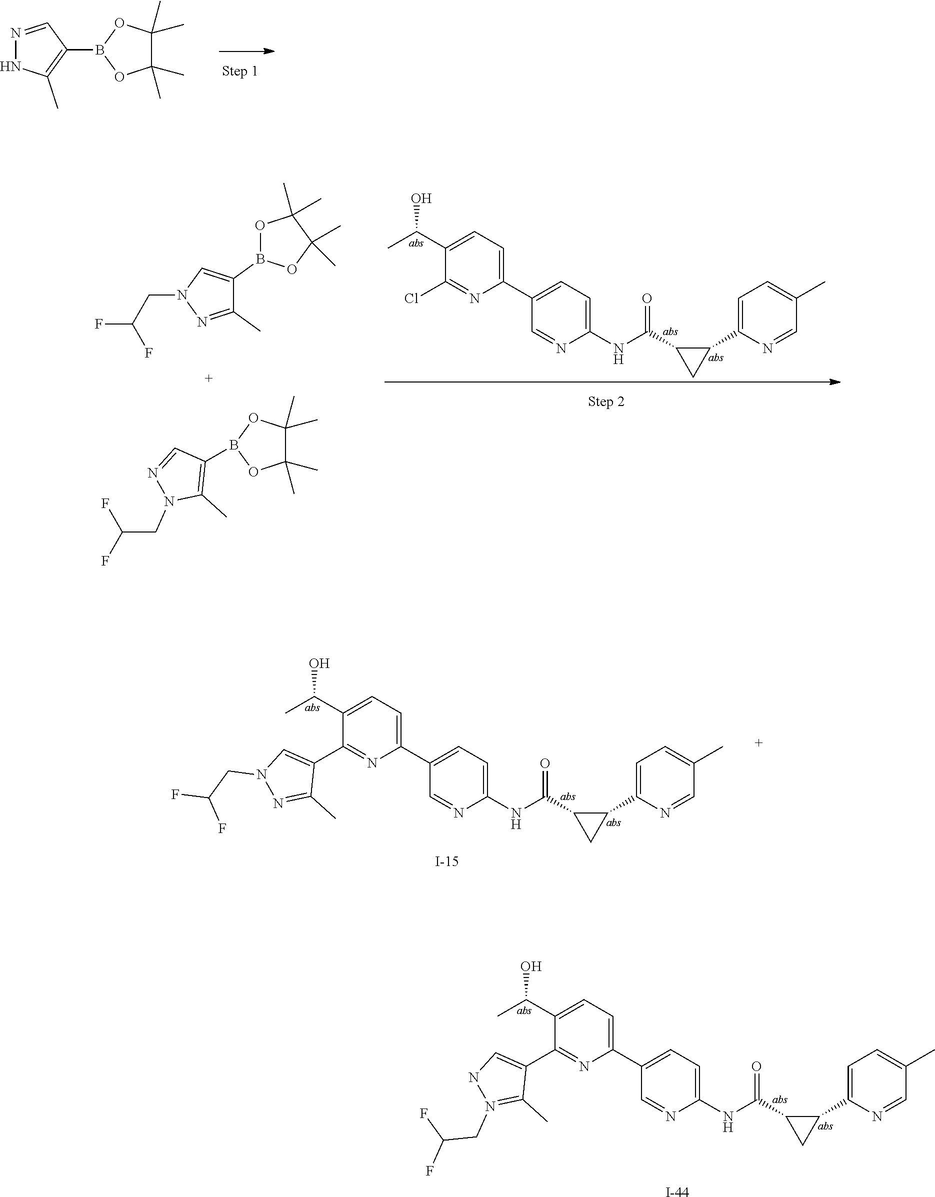

Step 1. Synthesis of 1-(2,2-difluoroethyl)-3-methyl-4-(4,4,5,5-tetramethyl-1,3,2-dioxaborolan-2-yl)-1H-pyrazole & 1-(2,2-difluoroethyl)-5-methyl-4-(4,4,5,5-tetramethyl-1,3,2-dioxaborolan-2-yl)-1H-pyrazole (mixture of two regioisomers). To a solution of 5-methyl-4-(4,4,5,5-tetramethyl-1,3,2-dioxaborolan-2-yl)-1H-pyrazole (500 mg, 2.40 mmol, 1 eq) in DMF (10 mL) was added Cs₂CO₃ (1.57 g, 4.81 mmol, 2 eq) and 2,2-difluoroethyl trifluoromethanesulfonate (1.03 g, 4.80 mmol, 2 eq). The mixture was stirred at 100° C. for 2 h. The reaction mixture was diluted into water (20 mL) and extracted with EtOAc (15 mL×3). The combined organic layers were washed with brine 20 mL (10 mL×2), dried over anhydrous Na₂SO₄, filtered and concentrated under reduced pressure to give a residue. The residue was purified by flash silica gel chromatography (Eluent of PE/EtOAc) to afford the title compound as a mixture of two regioisomers which was used without any further purification in the next step. LCMS: 273.1 [M+H]⁺.

Step 2. Synthesis of (1S,2R)—N-(6-(1-(2,2-difluoroethyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide & (1S,2R)—N-(6-(1-(2,2-difluoroethyl)-5-methyl-1H-pyrazol-4-yl)-5-((S)-1-hydroxyethyl)-[2,3′-bipyridin]-6′-yl)-2-(5-methylpyridin-2-yl)cyclopropane-1-carboxamide. To a solution of (1S,2R)—N-(6-chloro-5-((S)-1-hydroxyethyl)-[2,3′-bipyridin]-6′-yl)-2-(5-methylpyridin-2-yl)cyclopropane-1-carboxamide (50 mg, 122 mol, 1 eq) in dioxane (1 mL) and H₂O (0.2 mL) was added Pd(dppf)Cl₂·CH₂Cl₂ (10 mg, 12 mol, 0.1 eq), K₃PO₄ (65 mg, 306 mol, 2.5 eq) and 1-(2,2-difluoroethyl)-3-methyl-4-(4,4,5,5-tetramethyl-1,3,2-dioxaborolan-2-yl)-1H-pyrazole & 1-(2,2-difluoroethyl)-5-methyl-4-(4,4,5,5-tetramethyl-1,3,2-dioxaborolan-2-yl)-1H-pyrazole (mixture of two regioisomers) (50 mg, 183 mol, 1.5 eq). The mixture was stirred at 60° C. for 15 h. The reaction mixture was filtered, and the filtrate was concentrated under reduced pressure to give a residue. The residue was dissolved in MeOH (2 mL) and purified by reverse phase preparative HPLC (ACN/H₂O, 0.225% FA) to afford the title compound as a mixture of two regioisomers. The mixture of regioisomers was separated by chiral SFC (SFC Preparative Method: Instrument: Waters Waters 80 Preparative SFC system; Column: Daicel Chiralpak AS 250×25 mm I.D., 10 m particle size; Mobilephase A: CO₂, Mobile Phase B: EtOH(0.1% NH₄OH); Isocratic elution: 30% Phase B in Supercritical CO₂; Flow rate: 60 g/min; Retention time: Peak1: 3.77 min, Peak2: 5.15 min; Back Pressure: 100 bar to keep the CO₂ in Supercritical flow; Wave Length: 220 nm) to afford peak 1 (I-15) and peak 2 (I-44).

((1S,2R)—N-[5-[6-[1-(2,2-difluoroethyl)-3-methyl-pyrazol-4-yl]-5-[(1S)-1-hydroxyethyl]-2-pyridyl]-2-pyridyl]-2-(5-methyl-2-pyridyl)cyclopropanecarboxamide (Isomer 1). ¹H NMR (400 MHz, MeOD) δ (ppm): 8.92 (1H), 8.35-8.19 (2H), 8.12 (1H), 7.94-7.79 (3H), 7.56 (1H), 7.31 (1H), 6.49-6.01 (1H), 4.99 (1H), 4.61-4.51 (2H), 2.81-2.64 (1H), 2.55-2.41 (1H), 2.34 (3H), 2.30 (3H), 1.95-1.82 (1H), 1.58-1.47 (1H), 1.43 (3H). LCMS: 519.1 [M+H]⁺. Chiral SFC: Rt=1.194 min (instrument: Shimadzu LC-30AD; column: Chiralpak AS-3 50×4.6 mm I.D., particle size 3 μm; mobile phase A: CO₂; mobile phase B: EtOH with 0.05% diethylamine; 3.0 mL/min; 35° C.; 10 MPa, 220 nm).

((1S,2R)—N-[5-[6-[1-(2,2-difluoroethyl)-5-methyl-pyrazol-4-yl]-5-[(1S)-1-hydroxyethyl]-2-pyridyl]-2-pyridyl]-2-(5-methyl-2-pyridyl)cyclopropanecarboxamide (Isomer 2). ¹H NMR (400 MHz, MeOD) δ (ppm): 8.91 (1H), 8.30-8.22 (2H), 8.13 (1H), 7.96-7.82 (2H), 7.72 (1H), 7.56 (1H), 7.31 (1H), 6.52-6.02 (1H), 5.02 (1H), 4.66-4.59 (2H), 2.80-2.69 (1H), 2.52-2.44 (1H), 2.42 (3H), 2.30 (3H), 1.97-1.80 (1H), 1.59-1.46 (1H), 1.42 (3H). LCMS: 519.2 [M+H]⁺. Chiral SFC: Rt=1.352 min (instrument: Shimadzu LC-30AD; column: Chiralpak AS-3 50×4.6 mm I.D., particle size 3 μm; mobile phase A: CO₂; mobile phase B: EtOH with 0.05% diethylamine; 3.0 mL/min; 35° C.; 10 MPa, 220 nm).

### Example 5. Synthesis of (1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 1): (1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 2)

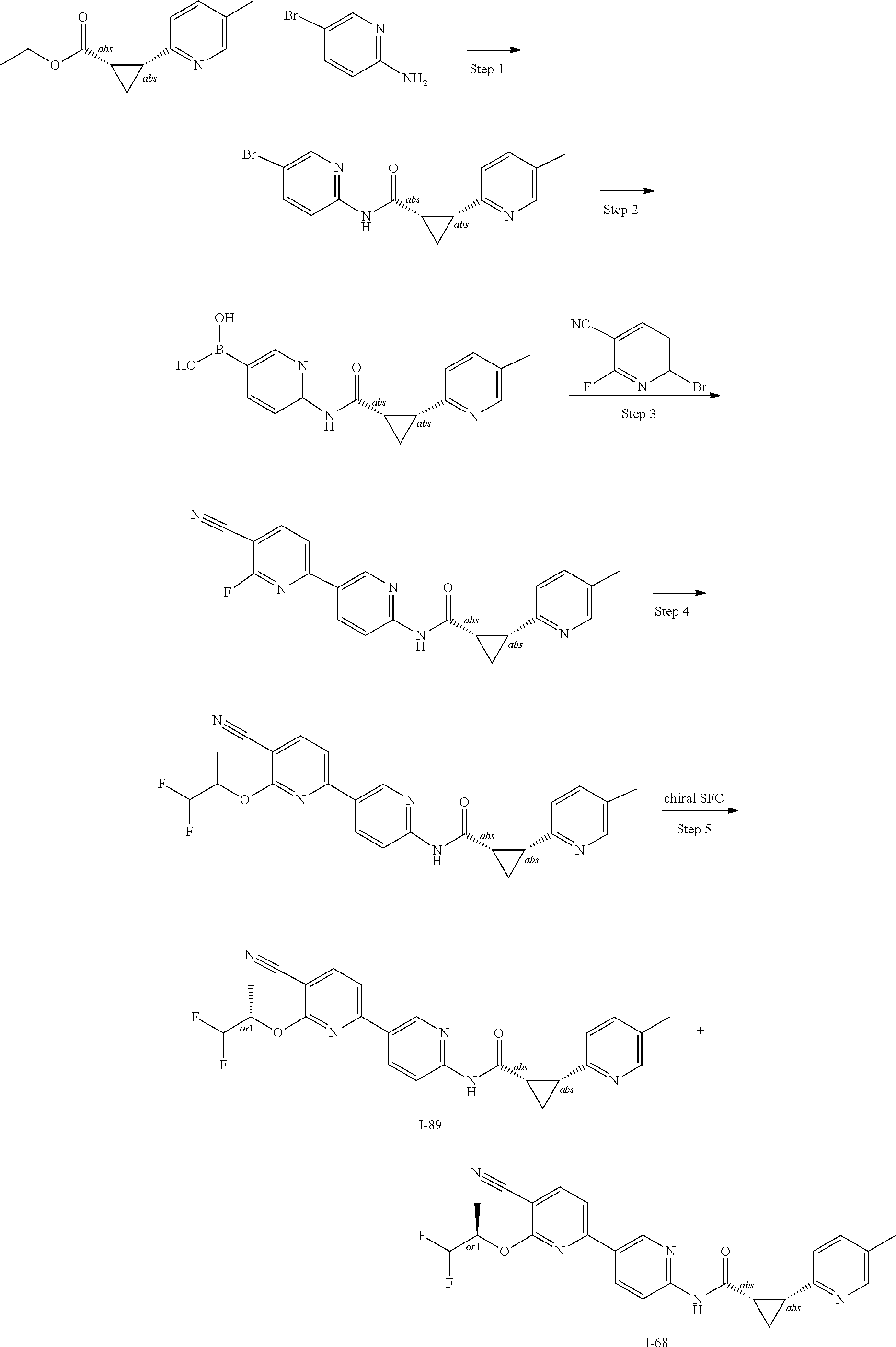

Step 1. Synthesis of (1S,2R)—N-(5-bromopyridin-2-yl)-2-(5-methylpyridin-2-yl) cyclopropane-1-carboxamide. A mixture of ethyl (1S,2R)-2-(5-methylpyridin-2-yl)cyclopropane-1-carboxylate (4.00 g, 19.49 mmol, 1 eq) and 5-bromopyridin-2-amine (3.37 g, 19.49 mmol, 1 eq) in toluene (50 mL) was degassed and purged with N₂ for 3 times and cooled to 0° C., and then LiHMDS (1 M in THF, 38.98 mL, 2 eq) was added to the solution at 0° C. under N₂ atmosphere. The mixture was warmed to 40° C. and stirred at 40° C. for 1 h under N₂ atmosphere. The reaction mixture was quenched by addition of saturated NH₄Cl (200 mL) and extracted with EtOAc (100 mL×3). The combined organic layers were washed with brine (100 mL×2), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 9.77 (1H), 8.38 (1H), 8.28 (1H), 7.97 (1H), 7.71-7.60 (1H), 7.48-7.40 (1H), 7.24 (1H), 2.73-2.58 (1H), 2.29 (3H), 2.26-2.18 (1H), 2.04-1.95 (1H), 1.64-1.50 (1H). LCMS: 332.0 [M+H]⁺.

Step 2. Synthesis of (6-((1S,2R)-2-(5-methylpyridin-2-yl)cyclopropane-1-carboxamido)pyridin-3-yl)boronic acid. A mixture of (1S,2R)—N-(5-bromopyridin-2-yl)-2-(5-methylpyridin-2-yl) cyclopropane-1-carboxamide (5.30 g, 15.95 mmol, 1 eq), 4,4,5,5-tetramethyl-2-(4,4,5,5-tetramethyl-1,3,2-dioxaborolan-2-yl)-1,3,2-dioxaborolane (6.08 g, 23.93 mmol, 1.5 eq), Pd(dppf)Cl₂·CH₂Cl₂ (1.30 g, 1.60 mmol, 0.1 eq) and KOAc (4.70 g, 47.86 mmol, 3 eq) in dioxane (70 mL) was degassed and purged with N₂ for 3 times, and then the mixture was stirred at 80° C. for 2 h under N₂ atmosphere. The reaction solution was filtered through diatomite and the filtrate was concentrated under reduced pressure to give a residue. The residue was purified by reverse phase preparative HPLC (ACN/H₂O, 0.1% FA) to afford the title compound. ¹H NMR (400 MHz, DMSO-d6) δ (ppm): 10.87 (1H), 8.60-8.43 (1H), 8.16 (1H), 7.98-7.69 (2H), 7.48-7.41 (1H), 7.17 (1H), 2.68-2.57 (1H), 2.48-2.40 (1H), 2.21 (3H), 1.74-1.64 (1H), 1.41-1.33 (1H). LCMS: 298.2 [M+H]⁺.

Step 3. Synthesis of (1S,2R)—N-(5-cyano-6-fluoro-[2,3′-bipyridin]-6′-yl)-2-(5-methylpyridin-2-yl)cyclopropane-1-carboxamide. A mixture of [6-[[(1S,2R)-2-(5-methyl-2-pyridyl)cyclopropanecarbonyl]amino]-3-pyridyl]boronic acid (500 mg, 1.68 mmol, 1 eq), 6-chloro-2-fluoro-pyridine-3-carbonitrile (263 mg, 1.68 mmol, 1 eq), K₃PO₄ (893 mg, 4.21 mmol, 2.5 eq) and Pd(dppf)Cl₂·CH₂Cl₂ (137 mg, 168.28 mol, 0.1 eq) in dioxane (5 mL) and H₂O (0.5 mL) was degassed and purged with N₂ for 3 times, and then the mixture was stirred at 40° C. for 1 h under N₂ atmosphere. The reaction mixture was diluted with water (10 mL) and extracted with EtOAc (10 mL×3). The combined organic layers were washed with brine (10 mL×2), dried over anhydrous sodium sulfate, filtered and concentrated under reduced pressure to give a residue. The residue was purified by reverse phase preparative HPLC (ACN/H₂O, 0.225% FA) to afford the title compound. ¹H NMR (400 MHz, CD₃OD) δ (ppm): 9.00 (1H), 8.40-8.28 (2H), 8.23 (1H), 8.02-7.89 (2H), 7.61-7.49 (1H), 7.31 (1H), 2.79-2.66 (1H), 2.50-2.41 (1H), 2.29 (3H), 1.92-1.84 (1H), 1.55-1.44 (1H). LCMS: 374.2 [M+H]⁺.

Step 4. Synthesis of (1S,2R)—N-(5-cyano-6-((1,1-difluoropropan-2-yl)oxy)-[2,3′-bipyridin]-6′-yl)-2-(5-methylpyridin-2-yl) cyclopropane-1-carboxamide. To a solution of 1,1-difluoropropan-2-ol (10 mg, 107 mol, 1 eq) in THF (0.5 mL) was added NaH (11 mg, 268 mol, 60% purity, 2.5 eq) at 0° C. Then (1S,2R)—N-[5-(5-cyano-6-fluoro-2-pyridyl)-2-pyridyl]-2-(5-methyl-2-pyridyl) cyclopropanecarboxamide (40 mg, 107 mol, 1 eq) was added to the mixture at 0° C. The mixture was stirred at 25° C. for 1 h. The reaction mixture was added dropwise into saturated NH₄Cl (2 mL) and extracted with DCM (3 mL×2). The combined organic layers were washed with brine (10 mL), dried over anhydrous Na₂SO₄, filtered and concentrated under reduced pressure to give a residue. The residue was purified by reverse phase preparative HPLC (ACN/H₂O, 0.05% NH₄OH) to afford the title compound. ¹H NMR (400 MHz, CD₃OD) δ (ppm): 9.00 (1H), 8.37-8.28 (1H), 8.22 (1H), 8.13 (1H), 7.94 (1H), 7.66 (1H), 7.58-7.48 (1H), 7.30 (1H), 6.28-5.92 (1H), 5.79-5.61 (1H), 2.78-2.67 (1H), 2.51-2.38 (1H), 2.29 (3H), 1.92-1.82 (1H), 1.55-1.44 (4H). LCMS: 450.2 [M+H]⁺. Chiral SFC: Rt=1.459 min & 1.673 min (49.4%: 50.5%) (instrument: SHIMADZU LC-30ADsf; column: Chiralpak AD-3 50×4.6 mm I.D., particle size 3 μm; mobile phase A: CO₂; mobile phase B: EtOH with 0.05% diethylamine; 3.0 mL/min; 35° C.; 10 MPa; 220 nm).

Step 5. Synthesis of (1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 1) & (1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 2). (1S,2R)—N-(5-cyano-6-((1,1-difluoropropan-2-yl)oxy)-[2,3′-bipyridin]-6′-yl)-2-(5-methylpyridin-2-yl) cyclopropane-1-carboxamide (mixture of two isomers) was purified by chiral SFC (SFC Preparative Method: Instrument: Waters 80Q Preparative SFC system; Column: DAICEL CHIRALPAK AD 250×30 mm I.D., 10 m particle size; Mobile phase A: CO₂, Mobile Phase B: EtOH (0.1% NH₄OH); Isocratic elution: 45% Phase B in Supercritical CO₂; Flow rate: 80 g/min; Retention time: Peak 1: 5.71 min, Peak 2: 8.04 min; Back Pressure: 100bar to keep the CO₂ in Supercritical flow; Wave Length: 220 nm) to give Peak 1 & Peak 2. Peak 1 was dissolved in DMF (1 mL) and purified by reverse phase preparative HPLC (ACN/H₂O, 0.05% NH₄OH) to afford the title compound (I-89, Isomer 1). Peak 2 was dissolved in DMF (1 mL) and purified by reverse phase preparative HPLC (ACN/H₂O, 0.05% NH₄OH) to afford the title compound (I-68, Isomer 2).

(1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 1). ¹H NMR (400 MHz, CD₃OD) δ (ppm): 9.00 (1H), 8.39-8.30 (1H), 8.23 (1H), 8.14 (1H), 7.94 (1H), 7.66 (1H), 7.59-7.50 (1H), 7.30 (1H), 6.30-5.92 (1H), 5.79-5.61 (1H), 2.79-2.67 (1H), 2.50-2.40 (1H), 2.29 (3H), 1.92-1.84 (1H), 1.53-1.47 (4H). LCMS: 450.2 [M+H]⁺. Chiral SFC: Rt=1.449 min (instrument: SHIMADZU LC-30ADsf; column: Chiralpak AD-3 50×4.6 mm I.D., particle size 3 μm; mobile phase A: CO₂; mobile phase B: EtOH with 0.05% diethylamine; 3.0 mL/min; 35° C.; 10 MPa; 220 nm).

(1S,2R)—N-(5-cyano-6-(((R*)-1,1-difluoropropan-2-yl)oxy)-[2,3′-bipyridin]-6′-yl)-2-(5-methylpyridin-2-yl)cyclopropane-1-carboxamide (Isomer 2). ¹H NMR (400 MHz, CD₃OD) δ (ppm): 9.00 (1H), 8.38-8.29 (1H), 8.23 (1H), 8.13 (1H), 7.94 (1H), 7.66 (1H), 7.58-7.50 (1H), 7.30 (1H), 6.28-5.92 (1H), 5.80-5.61 (1H), 2.78-2.68 (1H), 2.49-2.41 (1H), 2.29 (3H), 1.92-1.83 (1H), 1.54-1.47 (4H). LCMS: 450.3 [M+H]⁺. Chiral SFC: Rt=1.684 min (instrument: SHIMADZU LC-30ADsf; column: Chiralpak AD-3 50×4.6 mm I.D., particle size 3 μm; mobile phase A: CO₂; mobile phase B: EtOH with 0.05% diethylamine; 3.0 mL/min; 35° C.; 10 MPa; 220 nm).

### Example 6. Synthesis of (1S,2R)—N-(6-(3-cyano-5-methyl-1H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 1) & (1S,2R)—N-(6-(3-cyano-5-methyl-1H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 2)

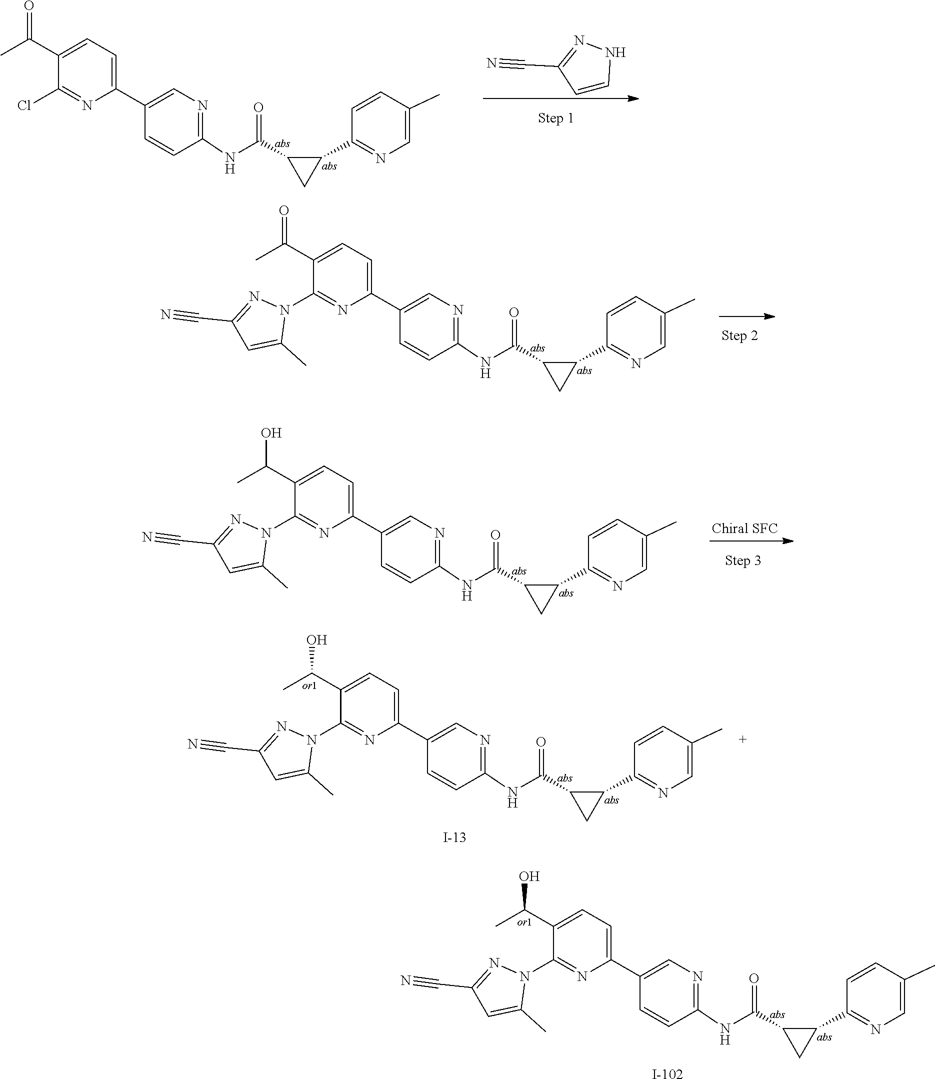

Step 1. Synthesis of (1S,2R)—N-(5-acetyl-6-(3-cyano-5-methyl-1H-pyrazol-1-yl)-[2,3′-bipyridin]-6′-yl)-2-(5-methylpyridin-2-yl) cyclopropane-1-carboxamide. To a solution of (1S,2R)—N-(5-acetyl-6-chloro-[2,3′-bipyridin]-6′-yl)-2-(5-methylpyridin-2-yl)cyclopropane-1-carboxamide (80 mg, 197 mol, 1 eq) in DMF (1 mL) was added Cs₂CO₃ (128 mg, 393 mol, 2 eq) and 5-methyl-1H-pyrazole-3-carbonitrile (63 mg, 590 mol, 3 eq). The mixture was stirred at 100° C. for 1 h. The reaction mixture was diluted with water (5 mL) and extracted with EtOAc (5 mL×3). The combined organic layers were washed with brine (10 mL×2), dried over anhydrous Na₂SO₄, filtered and concentrated under reduced pressure to give a residue. The residue was purified by flash silica gel chromatography (Eluent of PE/EtOAc & DCM/MeOH) to afford the title compound. LCMS: 478.3 [M+H]⁺.

Step 2. Synthesis of (1S,2R)—N-(6-(3-cyano-5-methyl-1H-pyrazol-1-yl)-5-(1-hydroxyethyl)-[2,3′-bipyridin]-6′-yl)-2-(5-methylpyridin-2-yl)cyclopropane-1-carboxamide. To a solution of (1S,2R)—N-(5-acetyl-6-(3-cyano-5-methyl-1H-pyrazol-1-yl)-[2,3′-bipyridin]-6′-yl)-2-(5-methylpyridin-2-yl) cyclopropane-1-carboxamide (80 mg, 168 mol, 1 eq) in MeOH (1 mL) was added NaBH₄ (6.34 mg, 167.53 mol, 1 eq) at 0° C., the mixture was warmed to 25° C. and stirred at 25° C. for 1 h. The reaction mixture was diluted with saturated NH₄Cl (10 mL) and extracted with EtOAc (5 mL×3). The combined organic layers were washed with brine (10 mL×2), dried over anhydrous Na₂SO₄, filtered and concentrated under reduced pressure to give a residue. The residue was purified by reverse phase preparative HPLC (ACN/H₂O, 0.05% NH₄OH) to afford the title compound. LCMS: 480.3 [M+H]⁺.

Step 3. Synthesis of (1S,2R)—N-(6-(3-cyano-5-methyl-1H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 1) & (1S,2R)—N-(6-(3-cyano-5-methyl-1H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 2). (1S,2R)—N-(6-(3-cyano-5-methyl-1H-pyrazol-1-yl)-5-(1-hydroxyethyl)-[2,3′-bipyridin]-6′-yl)-2-(5-methylpyridin-2-yl) cyclopropane-1-carboxamide (15 mg, 31.28 mol, 1 eq) was purified by chiral SFC (SFC Preparative Method: Instrument: Waters 80Q Preparative SFC system; Column: DAICEL Chiralpak AD 250×30 mm LD., 10 m particle size; Mobile phase A: CO₂, Mobile Phase B: IPA (0.1% NH₄OH); Isocratic elution: 45% Phase B in Supercritical CO₂; Flow rate: 70 g/min; Retention time: Peak 1: 3.31 min, Peak 2: 4.29 min; Back Pressure: 100 bar to keep the CO₂ in Supercritical flow; Wave Length: 220 nm) to give Peak 1 (I-13) and Peak 2 (I-102).

(1S,2R)—N-(6-(3-cyano-5-methyl-H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 1). ¹H NMR (400 MHz, CD₃OD) δ (ppm): 8.94 (1H), 8.36-8.27 (2H), 8.22 (1H), 8.11 (1H), 7.91 (1H), 7.58-7.50 (1H), 7.29 (1H), 6.81 (1H), 4.79-4.69 (1H), 2.78-2.67 (1H), 2.49-2.40 (1H), 2.36 (3H), 2.28 (3H), 1.91-1.83 (1H), 1.54-1.45 (1H), 1.36 (3H). LCMS: 480.3 [M+H]⁺. Chiral SFC: Rt=1.494 min (instrument: Waters 80Q Preparative SFC system; column: DAICEL Chiralpak AD 250×30 mm I.D., 10 μm particle size; mobile phase A: CO₂; mobile phase B: iPrOH with 0.1% NH₄OH; 70 g/min; 100 bar, 220 nm).

(1S,2R)—N-(6-(3-cyano-5-methyl-H-pyrazol-1-yl)-5-((R*)-1-hydroxyethyl)-[2,3′-bipyridin]-6′-yl)-2-(5-methylpyridin-2-yl)cyclopropane-1-carboxamide (Isomer 2). ¹H NMR (400 MHz, CD₃OD) δ (ppm): 8.94 (1H), 8.35-8.25 (2H), 8.22 (1H), 8.11 (1H), 7.91 (1H), 7.57-7.48 (1H), 7.29 (1H), 6.81 (1H), 4.78-4.68 (1H), 2.78-2.67 (1H), 2.50-2.40 (1H), 2.36 (3H), 2.28 (3H), 1.93-1.82 (1H), 1.54-1.46 (1H), 1.36 (3H). LCMS: 480.3 [M+H]⁺. Chiral SFC: Rt=1.639 min (instrument: Waters 80Q Preparative SFC system; column: DAICEL Chiralpak AD 250×30 mm I.D., 10 μm particle size; mobile phase A: CO₂; mobile phase B: iPrOH with 0.1% NH₄OH; 70 g/min; 100 bar, 220 nm).

### Example 7. Synthesis of (1S,2R)—N-(5-cyano-6-(3-(difluoromethoxy)-5-methyl-1H-pyrazol-1-yl)-[2,3′-bipyridin]-6′-yl)-2-(5-methylpyridin-2-yl)cyclopropane-1-carboxamide (I-96)

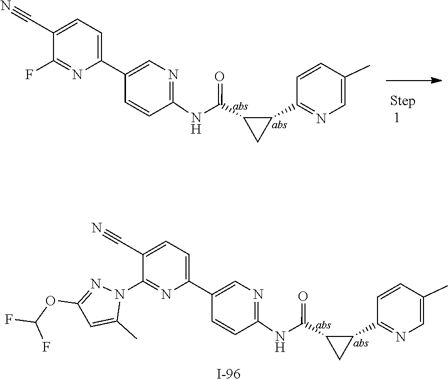

To a solution of (1S,2R)—N-(5-cyano-6-fluoro-[2,3′-bipyridin]-6′-yl)-2-(5-methylpyridin-2-yl)cyclopropane-1-carboxamide (35 mg, 94 mol, 1 eq) in DMF (0.5 mL) was added Cs₂CO₃ (61 mg, 187 mol, 2 eq) and 3-(difluoromethoxy)-5-methyl-1H-pyrazole (42 mg, 281 mol, 3 eq). The mixture was stirred at 100° C. for 1 h. The reaction solution was filtered through diatomite and the filtrate was concentrated under reduced pressure to give a residue. The residue was dissolved in DMF (2 mL) and purified by reverse phase preparative HPLC (ACN/H₂O, 0.2% FA) to afford the title compound. ¹H NMR (400 MHz, CD₃OD) δ (ppm): 9.03 (1H), 8.41-8.29 (2H), 8.23 (1H), 8.03-7.94 (2H), 7.58-7.52 (1H), 7.37-6.97 (2H), 6.09 (1H), 2.80-2.69 (1H), 2.67 (3H), 2.51-2.41 (1H), 2.29 (3H), 1.93-1.84 (1H), 1.55-1.45 (1H). LCMS: 502.3 [M+H]⁺.

### Example 8. Synthesis of (1S,2R)—N-(5-cyano-6-(3-(difluoromethoxy)-5-methyl-1H-pyrazol-1-yl)[2,3′-bipyridin]-6′-yl)-2-(5-methylpyridin-2-yl)cyclopropane-1-carboxamide (I-97)

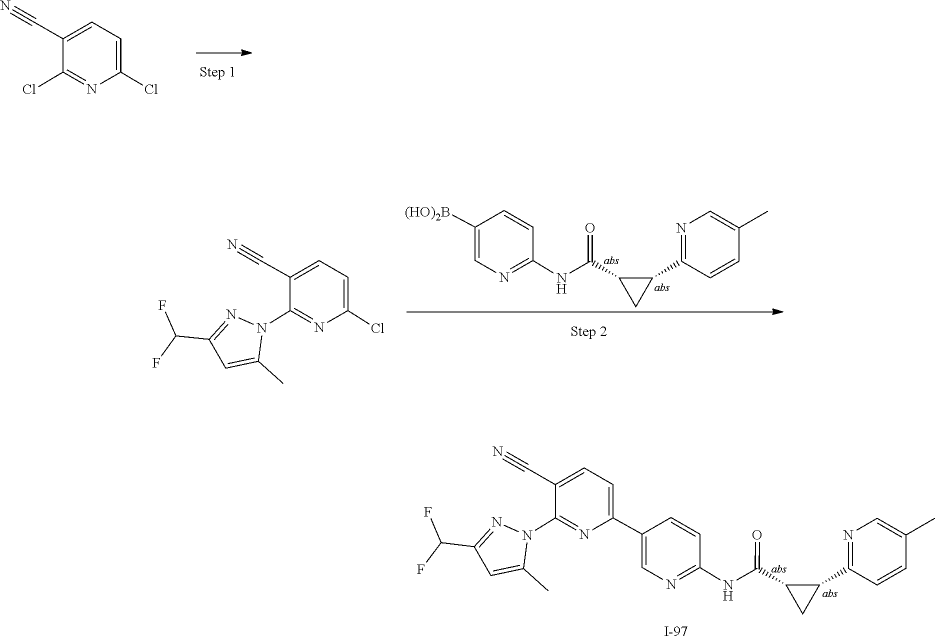

Step 1. Synthesis of 6-chloro-2-(3-(difluoromethyl)-5-methyl-1H-pyrazol-1-yl)nicotinonitrile. To a solution of 2,6-dichloronicotinonitrile (100 mg, 639 mol, 1 eq) in DMSO (1 mL) was added DIEA (248 mg, 1.92 mmol, 3 eq) and 3-(difluoromethyl)-5-methyl-1H-pyrazole (80 mg, 607 mol, 0.95 eq). The mixture was stirred at 80° C. for 4 h. The reaction mixture was added into water (10 mL) and extracted with EtOAc (3 mL×3). The combined organic layers were washed with brine (5 mL×2), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CD₃OD) δ (ppm): 8.38 (1H), 7.68 (1H), 6.95-6.53 (2H), 2.60 (3H). LCMS: 269.1 [M+H]⁺.

Step 2. Synthesis of (1S,2R)—N-(5-cyano-6-(3-(difluoromethyl)-5-methyl-1H-pyrazol-1-yl)-[2,3′-bipyridin]-6′-yl)-2-(5-methylpyridin-2-yl)cyclopropane-1-carboxamide (I-97). To a solution of (6-((1S,2R)-2-(5-methylpyridin-2-yl)cyclopropane-1-carboxamido)pyridin-3-yl)boronic acid (33 mg, 112 mol, 1 eq) in dioxane (0.5 mL) and H₂O (0.1 mL) was added Pd(dppf)Cl₂·CH₂Cl₂ (9 mg, 11 mol, 0.1 eq) and K₃PO₄ (59.26 mg, 279.17 mol, 2.5 eq) and 6-chloro-2-(3-(difluoromethyl)-5-methyl-1H-pyrazol-1-yl)nicotinonitrile (30 mg, 112 mol, 1 eq). The mixture was stirred at 60° C. for 15 h. The reaction mixture was concentrated under reduced pressure to give a residue. The residue was dissolved in DMF (1 mL) and purified by reverse phase preparative HPLC (ACN/H₂O, 0.2% FA) to afford the title compound. ¹H NMR (400 MHz, CD₃OD) δ (ppm): 9.04 (1H), 8.41 (1H), 8.39-8.35 (1H), 8.23 (1H), 8.09 (1H), 7.98 (1H), 7.59-7.52 (1H), 7.30 (1H), 6.80 (1H), 6.60 (1H), 2.78-2.69 (1H), 2.63 (3H), 2.50-2.41 (1H), 2.29 (3H), 1.92-1.84 (1H), 1.55-1.45 (1H). LCMS: 486.2 [M+H]⁺.

### Example 9. Synthesis of (1S,2R)—N-(5-cyano-6-((1s,3R)-3-hydroxy-3-methylcyclobutoxy)-[2,3′-bipyridin]-6′-yl)-2-(5-methylpyridin-2-yl) cyclopropane-1-carboxamide (I-72)

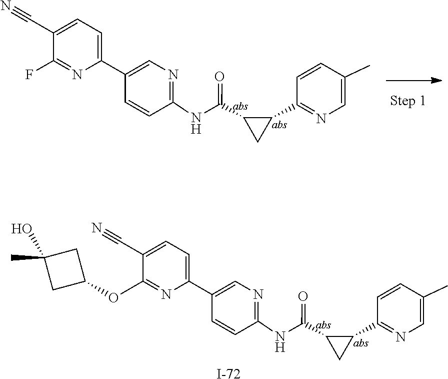

To a solution of 1-methylcyclobutane-1,3-diol (16 mg, 161 mol, 1.5 eq) in THF (0.5 mL) was added NaH (11 mg, 268 mol, 60% purity, 2.5 eq) at 0° C. Then (1S,2R)—N-(5-cyano-6-fluoro-[2,3′-bipyridin]-6′-yl)-2-(5-methylpyridin-2-yl)cyclopropane-1-carboxamide (40 mg, 107 mol, 1 eq) was added to the mixture at 0° C. The mixture was stirred at 25° C. for 1 h. The reaction mixture was added with saturated NH₄Cl solution (2 mL) and extracted with DCM (3 mL×2). The combined organic layers were washed with brine (10 mL), dried over anhydrous Na₂SO₄, filtered and concentrated under reduced pressure to give a residue. The residue was dissolved in DMF (2 mL) and purified by reverse phase preparative HPLC (ACN/H₂O, 0.05% NH₄OH) to afford the title compound. ¹H NMR (400 MHz, DMSO-d6) δ (ppm): 10.98 (1H), 9.03 (1H), 8.44-8.34 (1H), 8.29 (1H), 8.22 (1H), 7.95 (1H), 7.75 (1H), 7.52-7.39 (1H), 7.19 (1H), 5.34-5.10 (1H), 5.08-4.94 (1H), 2.72-2.64 (1H), 2.64-2.56 (2H), 2.46 (1H), 2.21 (5H), 1.77-1.68 (1H), 1.45-1.36 (1H), 1.30 (3H). LCMS: 456.3 [M+H]⁺.

### Example 10. Synthesis of (1S,2R)—N-(6-(3-cyano-5-methyl-1H-pyrazol-1-yl)-5-(difluoromethyl)-[2,3′-bipyridin]-6′-yl)-2-(5-methylpyridin-2-yl)cyclopropane-1-carboxamide (I-94)

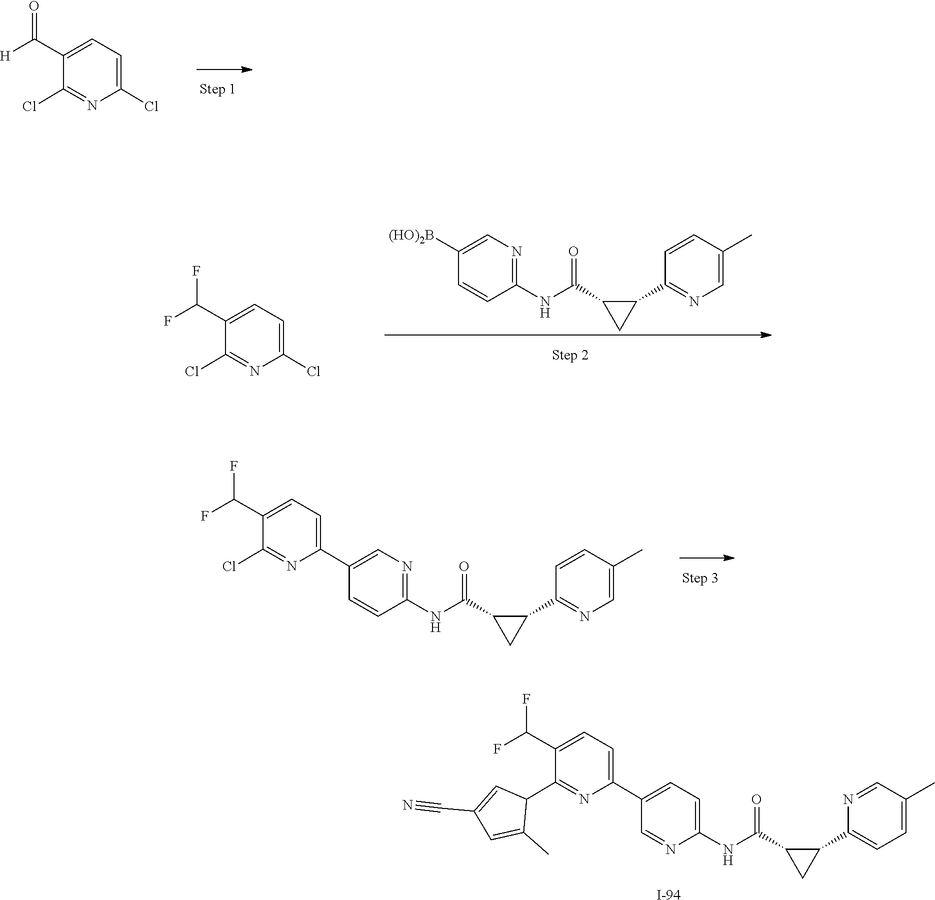

Step 1. Synthesis of 2,6-dichloro-3-(difluoromethyl)pyridine. To a solution of 2,6-dichloropyridine-3-carbaldehyde (5.00 g, 28.41 mmol, 1 eq) in DCM (25 mL) was added DAST (13.74 g, 85.23 mmol, 11.26 mL, 3 eq) at 0° C. The mixture was stirred at 0° C. for 1 h. Then the reaction mixture was added into saturated Na₂CO₃ solution (30 mL) at 0° C. and extracted with DCM (50 mL×3). The combined organic layers were washed with brine (30 mL), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 7.97 (1H), 7.43 (1H), 6.90 (1H).

Step 2. Synthesis of (1S,2R)—N-(6-chloro-5-(difluoromethyl)-[2,3′-bipyridin]-6′-yl)-2-(5-methylpyridin-2-yl)cyclopropane-1-carboxamide. A mixture of (6-((1S,2R)-2-(5-methylpyridin-2-yl)cyclopropane-1-carboxamido)pyridin-3-yl)boronic acid (500 mg, 1.68 mmol, 1 eq), 2,6-dichloro-3-(difluoromethyl)pyridine (500 mg, 2.52 mmol, 1.5 eq), Pd(dppf)Cl₂·CH₂Cl₂ (137 mg, 168 mol, 0.1 eq) and K₃PO₄ (893 mg, 4.21 mmol, 2.5 eq) in dioxane (5 mL) and H₂O (1 mL) was degassed and purged with N₂ for 3 times, and then the mixture was warmed to 40° C. and stirred at 40° C. for 2 h under N₂ atmosphere. The reaction mixture was concentrated under reduced pressure to give a residue. The residue was purified by flash silica gel chromatography (Eluent of PE/EtOAc) to afford the title compound. ¹H NMR (400 MHz, DMSO-d6) δ (ppm): 11.03 (1H), 9.01 (1H), 8.38-8.32 (1H), 8.21 (2H), 8.17-8.12 (1H), 7.95 (1H), 7.49-7.43 (1H), 7.39-7.05 (2H), 2.70-2.62 (1H), 2.49-2.43 (1H), 2.21 (3H), 1.77-1.69 (1H), 1.43-1.34 (1H). LCMS: 415.1 [M+H]⁺.

Step 3. Synthesis of (1S,2R)—N-(6-(3-cyano-5-methyl-1H-pyrazol-1-yl)-5-(difluoromethyl)-[2,3′-bipyridin]-6′-yl)-2-(5-methylpyridin-2-yl)cyclopropane-1-carboxamide (I-94). To a solution of (1S,2R)—N-[5-[6-chloro-5-(difluoromethyl)-2-pyridyl]-2-pyridyl]-2-(5-methyl-2-pyridyl)cyclopropanecarboxamide (50 mg, 121 mol, 1 eq) and 5-methyl-1H-pyrazole-3-carbonitrile (19 mg, 181 mol, 1.5 eq) in DMF (0.5 mL) was added Cs₂CO₃ (79 mg, 241 mol, 2 eq). The mixture was warmed to 100° C. and stirred at 100° C. for 12 h. The reaction mixture was filtered and washed by DMF (1 mL). The filtrate was purified by reverse phase preparative HPLC (MeCN/H₂O, 0.2% FA) to afford the title compound. ¹H NMR (400 MHz, CD₃OD) δ (ppm): 9.03 (1H), 8.43-8.31 (2H), 8.24 (1H), 8.22-8.15 (1H), 7.98 (1H), 7.57 (1H), 7.32 (1H), 7.06 (1H), 6.86 (1H), 2.79-2.71 (1H), 2.51 (3H), 2.49-2.43 (1H), 2.31 (3H), 1.93-1.86 (1H), 1.55-1.47 (1H). LCMS: 486.1 [M+H]⁺.

### Example 11. Synthesis of (1S,2R)—N-(5-((S)-1-hydroxyethyl)-6-(5-methyl-1-(2,2,2-trifluoroethyl)-1H-pyrazol-4-yl)-[2,3′-bipyridin]-6′-yl)-2-(5-methylpyridin-2-yl)cyclopropane-1-carboxamide (I-31)

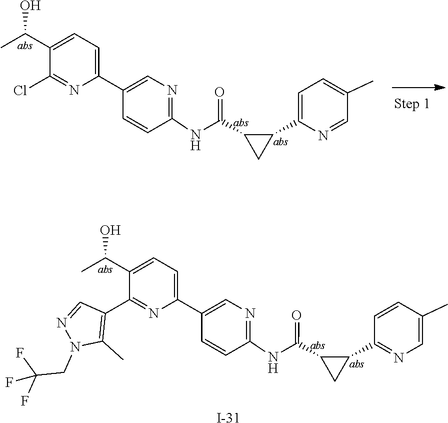

A mixture of (1S,2R)—N-(6-chloro-5-((S)-1-hydroxyethyl)-[2,3′-bipyridin]-6′-yl)-2-(5-methylpyridin-2-yl)cyclopropane-1-carboxamide (30 mg, 73 mol, 1 eq), 5-methyl-4-(4,4,5,5-tetramethyl-1,3,2-dioxaborolan-2-yl)-1-(2,2,2-trifluoroethyl)pyrazole (21 mg, 73 mol, 1 eq), Pd(dppf)Cl₂·CH₂Cl₂ (6 mg, 7 mol, 0.1 eq) and K₃PO₄ (39 mg, 183 mol, 2.5 eq) in dioxane (0.5 mL) and H₂O (0.1 mL) was degassed and purged with N₂ for 3 times, and then the mixture was warmed to 60° C. and stirred at 60° C. for 2 h under N₂ atmosphere. The reaction mixture was concentrated under reduced pressure to give a residue. The residue was dissolved in MeOH (1.5 mL) and purified by reverse phase preparative HPLC (ACN/H₂O, 0.05% NH₄OH) and preparative TLC (SiO₂, Eluent of PE/EA/MeOH=10:10:1) to afford the title compound. ¹H NMR (400 MHz, CD₃OD) δ (ppm): 8.89 (1H), 8.27-8.20 (2H), 8.11 (1H), 7.89 (1H), 7.83 (1H), 7.73 (1H), 7.57-7.51 (1H), 7.29 (1H), 5.05-4.95 (3H), 2.76-2.68 (1H), 2.48-2.42 (1H), 2.41 (3H), 2.28 (3H), 1.91-1.83 (1H), 1.52-1.45 (1H), 1.40 (3H). LCMS: 537.2 [M+H]⁺.

### Example 12. Synthesis of (1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 1) and (1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 2)

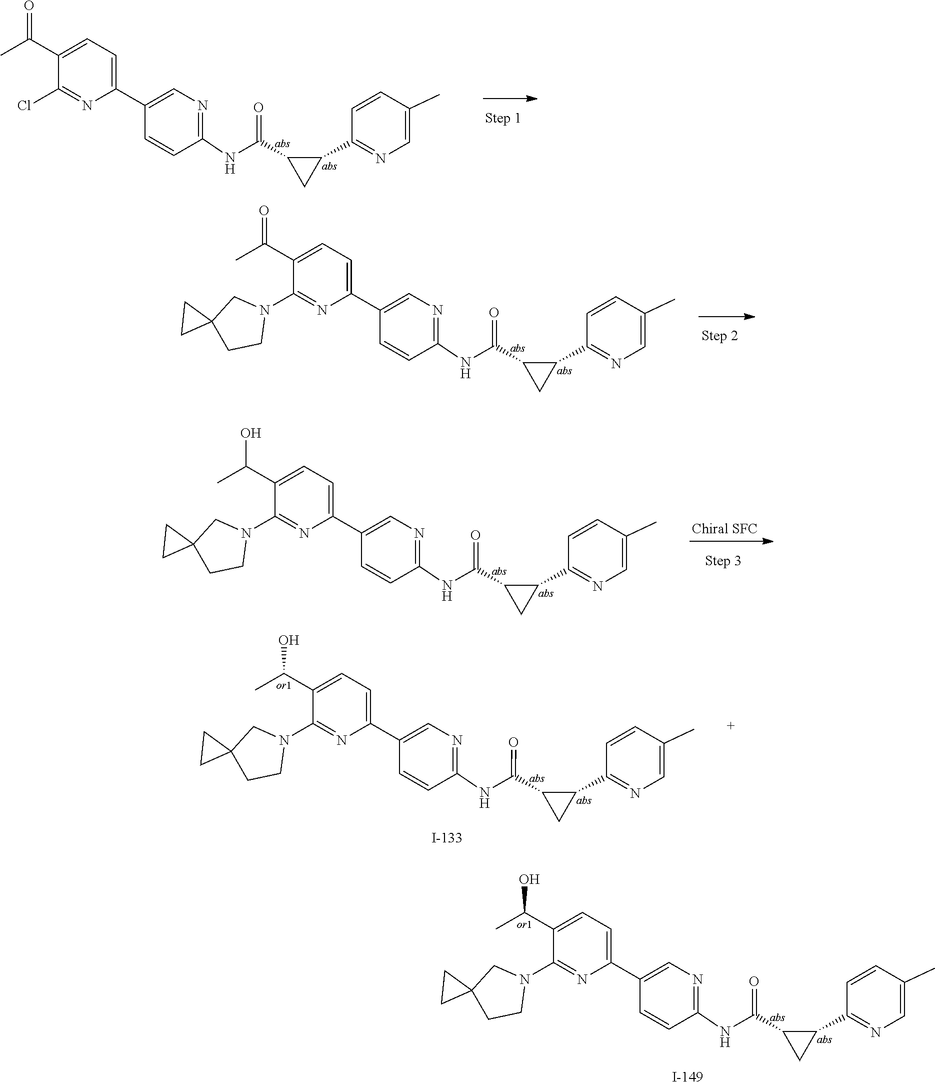

Step 1. Synthesis of (1S,2R)—N-(5-acetyl-6-(5-azaspiro[2.4]heptan-5-yl)-[2,3′-bipyridin]-6′-yl)-2-(5-methylpyridin-2-yl)cyclopropane-1-carboxamide. To a solution of (1S,2R)—N-(5-acetyl-6-chloro-[2,3′-bipyridin]-6′-yl)-2-(5-methylpyridin-2-yl)cyclopropane-1-carboxamide (100 mg, 246 mol, 1 eq) in DMF (1 mL) was added Cs₂CO₃ (160 mg, 492 mol, 2 eq) and 5-azaspiro[2.4]heptane (66 mg, 492 mol, 2 eq, HCl). The mixture was stirred at 100° C. for 1 h. The reaction mixture was diluted with water (2 mL) and extracted with EtOAc (3 mL×3). The combined organic layers were washed with brine (5 mL×2), dried over anhydrous Na₂SO₄, filtered and concentrated under reduced pressure to give a residue. The residue was purified by flash silica gel chromatography (Eluent of PE/EtOAc & DCM/MeOH) to afford the title compound. ¹H NMR (400 MHz, CDCl₃) δ (ppm): 9.64 (1H), 8.96 (1H), 8.39 (1H), 8.28-8.21 (1H), 8.10 (1H), 8.03 (1H), 7.88 (1H), 7.48-7.41 (1H), 7.03 (1H), 3.58 (2H), 2.76-2.66 (1H), 2.57 (3H), 2.30 (3H), 2.28-2.20 (1H), 2.08-2.00 (1H), 1.88 (2H), 1.78-1.68 (2H), 1.62-1.56 (1H), 0.62 (4H). LCMS: 468.2 [M+H]⁺.

Step 2. Synthesis of (1S,2R)—N-(5-(1-hydroxyethyl)-6-(5-azaspiro[2.4]heptan-5-yl)-[2,3′-bipyridin]-6′-yl)-2-(5-methylpyridin-2-yl)cyclopropane-1-carboxamide (mixture of two diastereomers). To a solution of (1S,2R)—N-(5-acetyl-6-(5-azaspiro[2.4]heptan-5-yl)-[2,3′-bipyridin]-6′-yl)-2-(5-methylpyridin-2-yl)cyclopropane-1-carboxamide (110 mg, 235 mol, 1 eq) in MeOH (1.5 mL) was added NaBH₄ (9 mg, 235 mol, 1 eq) at 0° C., the mixture was warmed to 25° C. and stirred at 25° C. for 1 h. The reaction mixture was quenched with saturated NH₄Cl solution (10 mL) and extracted with EtOAc (5 mL×3). The combined organic layers were washed with brine (10 mL×2), dried over anhydrous Na₂SO₄, filtered and concentrated under reduced pressure to give a residue. The residue was purified by reverse phase preparative HPLC (ACN/H₂O, 0.05% NH₄OH) to afford the title compound. LCMS: 470.3 [M+H]⁺.

Step 3. Synthesis of (1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 1) and (1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 2). (1S,2R)—N-(5-(1-hydroxyethyl)-6-(5-azaspiro[2.4]heptan-5-yl)-[2,3′-bipyridin]-6′-yl)-2-(5-methylpyridin-2-yl)cyclopropane-1-carboxamide (mixture of two diastereomers) was separated by chiral SFC (SFC Preparative Method: Instrument: Waters 150 Preparative SFC system; Column: Daicel Chiralpak WHELK-O1 250×30 mm I.D., 10 m particle size; Mobilephase A: CO₂, Mobile Phase B: MeOH (0.2% NH₄OH); Isocratic elution: 45% Phase B in Supercritical CO₂; Flow rate: 120 g/min; Retention time: Peak 1: 4.52 min, Peak 2: 6.21 min; Back Pressure: 80bar to keep the CO₂ in Supercritical flow; Wave Length: 220 nm) give the first (peak 1) & second (peak 2) eluting compound. The first (peak 1) eluting compound was dissolved in DMSO (1 mL) and purified by reverse phase preparative HPLC (ACN/H₂O, 0.05% NH₄OH) to afford the title compound (Isomer 1, I-133). The second (peak 2) eluting compound was dissolved in DMSO (1 mL) and purified by reverse phase preparative HPLC (ACN/H₂O, 0.05% NH₄OH) to afford the title compound (Isomer 2, I-149).

(1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 1). ¹H NMR (400 MHz, CD₃OD) δ (ppm): 8.92 (1H), 8.32-8.17 (2H), 7.89-7.77 (2H), 7.60-7.47 (1H), 7.34-7.20 (2H), 5.27-5.12 (1H), 3.88-3.77 (1H), 3.76-3.67 (1H), 3.62 (1H), 3.36 (1H), 2.79-2.65 (1H), 2.49-2.36 (1H), 2.29 (3H), 2.05-1.93 (1H), 1.90-1.74 (2H), 1.53-1.46 (1H), 1.43 (3H), 0.72-0.57 (4H). LCMS: 470.3 [M+H]⁺. Chiral SFC: Rt=1.381 min (instrument: SHIMADZU LC-30ADsf; column: (S,S)Whelk-O1 50×4.6 mm I.D., 3.5 μm; mobile phase A: CO₂; mobile phase B: MeOH (0.05% diethylamine); 3.0 mL/min; 35° C., 100 bar, 220 nm).

(1S,2R)—N-(5-((R*)-1-hydroxyethyl)-6-(5-azaspiro[2.4]heptan-5-yl)-[2,3′-bipyridin]-6′-yl)-2-(5-methylpyridin-2-yl)cyclopropane-1-carboxamide (Isomer 2). ¹H NMR (400 MHz, CD₃OD) δ (ppm): 8.92 (1H), 8.31-8.20 (2H), 7.87-7.78 (2H), 7.58-7.50 (1H), 7.34-7.22 (2H), 5.26-5.15 (1H), 3.87-3.77 (1H), 3.76-3.68 (1H), 3.62 (1H), 3.36 (1H), 2.77-2.66 (1H), 2.49-2.38 (1H), 2.29 (3H), 2.05-1.93 (1H), 1.91-1.75 (2H), 1.54-1.45 (1H), 1.43 (3H), 0.72-0.56 (4H). LCMS: 470.3 [M+H]⁺. Chiral SFC: Rt=1.699 min (instrument: SHIMADZU LC-30ADsf; column: (S,S)Whelk-O1 50×4.6 mm I.D., 3.5 μm; mobile phase A: CO₂; mobile phase B: MeOH (0.05% diethylamine); 3.0 mL/min; 35° C., 100 bar, 220 nm).

### Example 13. Synthesis of (1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 1) and (1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 2)

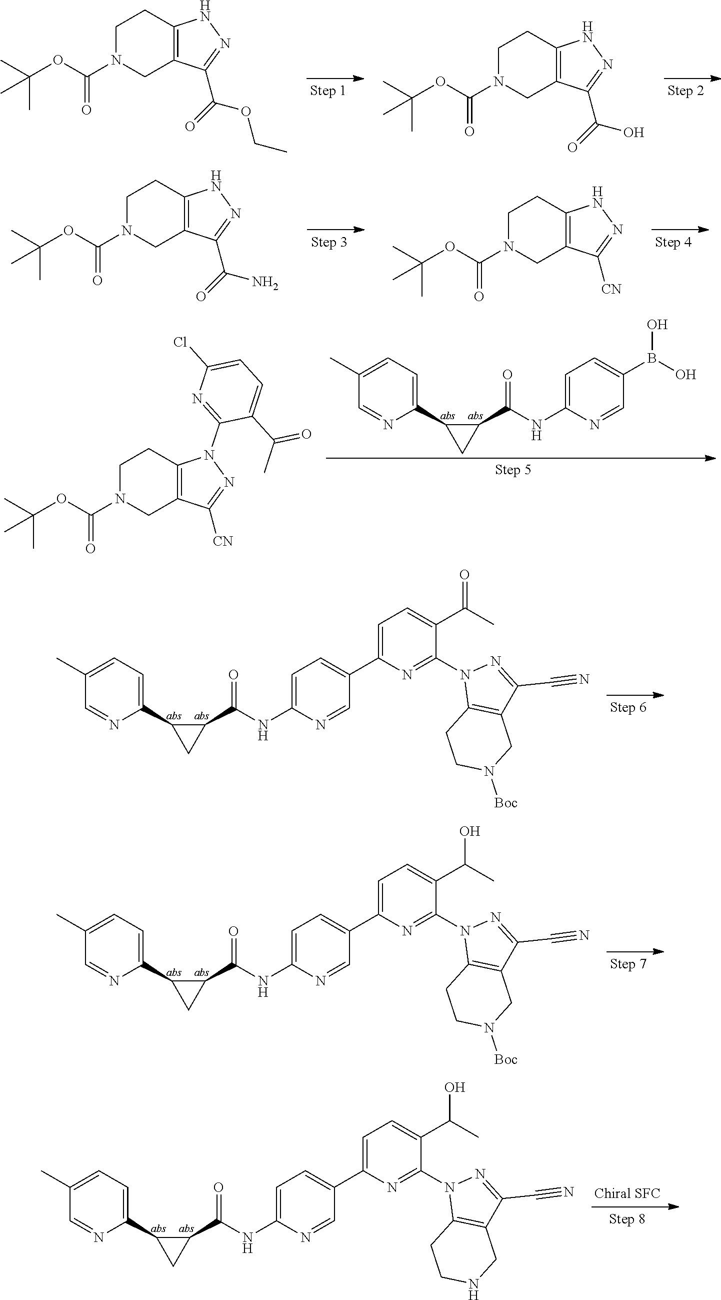

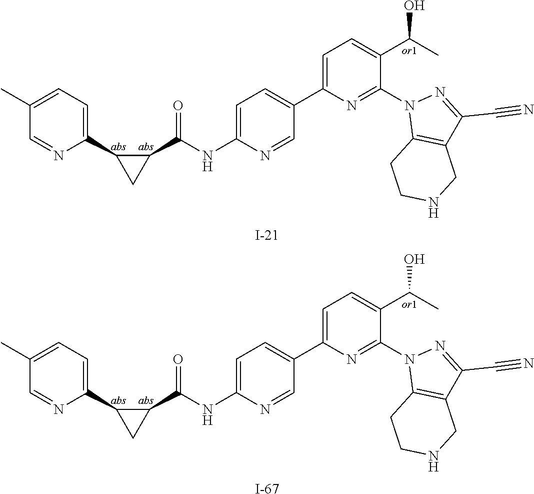

Step 1. Synthesis of 5-(tert-butoxycarbonyl)-4,5,6,7-tetrahydro-1H-pyrazolo[4,3-c]pyridine-3-carboxylic acid. To a stirred solution of 5-(tert-butyl) 3-ethyl 1,4,6,7-tetrahydro-5H-pyrazolo[4,3-c]pyridine-3,5-dicarboxylate (10 g, 33.9 mmol) in THF (50 mL) and MeH (25 mL, 617 mmol) was added Na H (100 mL, 100 mmol, 1.0 M in water) at RT. The resulting mixture was stirred at 60° C. overnight. The mixture was treated with HCl (12 mL, 1.0 M in water). The precipitated solids were collected by filtration, washed with water (2×20 ml) and dried under reduced pressure to provide the title product (8.8 g, 97%) as a yellow solid which was used in the next step without further purification. LCMS: 268 [M+H]⁺.

Step 2. Synthesis of tert-butyl 3-carbamoyl-1,4,6,7-tetrahydro-5H-pyrazolo[4,3-c]pyridine-5-carboxylate. To a stirred solution of 5-(tert-butoxycarbonyl)-4,5,6,7-tetrahydro-1H-pyrazolo[4,3-c]pyridine-3-carboxylic acid (5 g, 18.7 mmol) in DMF (10 mL) were added CDI (3.0 g, 18.7 mmol) at RT under nitrogen atmosphere. The resulting mixture was stirred for 2.0 h. Then NH₄OH (15 mL, 25% w/w in water) was added and stirred for additional 1 h. The reaction mixture was extracted with DCM (3×50 mL). The combined organic layers were dried over Na₂SO₄, filtered, and concentrated under reduced pressure. The residue was purified via reverse flash chromatography (C18 silica gel) using a 20% to 30% ACN in water (with 10 mmol/L NH₄HCO₃) gradient to provide the title compound (3.5 g, 65%) as a white solid. LCMS: 267 [M+H]⁺.

Step 3. Synthesis of tert-butyl 3-cyano-1,4,6,7-tetrahydro-5H-pyrazolo[4,3-c]pyridine-5-carboxylate. To a stirred solution of tert-butyl 3-carbamoyl-1,4,6,7-tetrahydro-5H-pyrazolo[4,3-c]pyridine-5-carboxylate (600 mg, 2.25 mmol) and DIEA (874 mg, 6.80 mmol) in DCM (20 mL) was added TFFA (1.79 g, 6.75 mmol) dropwise at 0° C. The resulting mixture was stirred at room temperature for 2.0 h. The reaction mixture was quenched with ice-water (50 mL) and extracted with EtOAc (3×50 mL). The combined organic layers were dried over Na₂SO₄, filtered, and concentrated under reduced pressure. The residue was purified via reverse flash chromatography (C18 silica gel) using a 0% to 50% ACN in water (with 10 mmol/L NH₄HCO₃) gradient to provide the title compound (180 mg, 32%) as a white solid. LCMS: 249 [M+H]⁺.

Step 4. Synthesis of tert-butyl 1-(3-acetyl-6-chloropyridin-2-yl)-3-cyano-1,4,6,7-tetrahydro-5H-pyrazolo[4,3-c]pyridine-5-carboxylate. To a stirred solution of 1-(6-chloro-2-fluoropyridin-3-yl) ethan-1-one (131 mg, 0.754 mmol) and DIEA (266 mg, 2.06 mmol) in DMSO (2.0 mL) were added tert-butyl 3-cyano-1,4,6,7-tetrahydro-5H-pyrazolo[4,3-c]pyridine-5-carboxylate (170 mg, 0.685 mmol) at RT under nitrogen atmosphere. The resulting mixture was stirred at 80° C. for 2.0 h. The reaction mixture was cooled, diluted with water (30 mL) and extracted with EtOAc (3×30 mL). The combined organic layers were dried over Na₂SO₄, filtered, and concentrated under reduced pressure. The residue was purified via reverse flash chromatography (C18 silica gel) using a 40% to 60% ACN in water (with 10 mmol/L NH₄HCO₃) gradient to provide the title compound (256 mg, 93%) as a yellow solid. LCMS: 402 [M+H]⁺.

Step 5. Synthesis of tert-butyl 1-(5-acetyl-6′-((1S,2R)-2-(5-methylpyridin-2-yl) cyclopropane-1-carboxamido)-[2,3′-bipyridin]-6-yl)-3-cyano-1,4,6,7-tetrahydro-5H-pyrazolo[4,3-c]pyridine-5-carboxylate. To a stirred solution of tert-butyl 1-(3-acetyl-6-chloropyridin-2-yl)-3-cyano-1,4,6,7-tetrahydro-5H-pyrazolo[4,3-c]pyridine-5-carboxylate (236 mg, 0.587 mmol) and (6-((1S,2R)-2-(5-methylpyridin-2-yl) cyclopropane-1-carboxamido) pyridin-3-yl) boronic acid (262 mg, 0.704 mmol) in dioxane (5 mL) and H₂O (0.5 mL) were added Pd(dppf)Cl₂·CH₂Cl₂ (48 mg, 0.059 mmol) at RT under nitrogen atmosphere. The resulting mixture was stirred at 80° C. for 1 h. The reaction mixture was cooled, diluted with water (30 mL) and extracted with EtOAc (3×30 mL). The combined organic layers were dried over Na₂SO₄, filtered, and concentrated under reduced pressure. The residue was purified via reverse flash chromatography (C18 silica gel) using a 40% to 60% ACN in water (with 10 mmol/L NH₄HCO₃) gradient to provide the title compound (220 mg, 60%) as a yellow solid. LCMS: 619 [M+H]⁺.

Step 6. Synthesis of tert-butyl 3-cyano-1-(5-(1-hydroxyethyl)-6′-((1S,2R)-2-(5-methylpyridin-2-yl) cyclopropane-1-carboxamido)-[2,3′-bipyridin]-6-yl)-1,4,6,7-tetrahydro-5H-pyrazolo[4,3-c]pyridine-5-carboxylate. To a stirred solution of tert-butyl 1-(5-acetyl-6′-((1S,2R)-2-(5-methylpyridin-2-yl) cyclopropane-1-carboxamido)-[2,3′-bipyridin]-6-yl)-3-cyano-1,4,6,7-tetrahydro-5H-pyrazolo[4,3-c]pyridine-5-carboxylate (200 mg, 0.323 mmol) in MeOH (1 mL) and THF (4 mL) was added NaBH₄ (62 mg, 1.62 mmol) at 0° C. under nitrogen atmosphere. The resulting mixture was stirred at room temperature for 1.0 h. The reaction was quenched by the addition of ice-water (10 mL) at 0° C. and extracted with EtOAc (2×20 mL). The combined organic layers were dried over Na₂SO₄, filtered, and concentrated under reduced pressure. The residue was purified via reverse flash chromatography (C18 silica gel) using a 0% to 40% ACN in water (with 10 mmol/L NH₄HCO₃) gradient to provide the title compound (150 mg, 75%) as a yellow solid. LCMS: 621 [M+H]⁺.

Step 7. Synthesis of (1S,2R)—N-(6-(3-cyano-4,5,6,7-tetrahydro-1H-pyrazolo[4,3-c]pyridin-1-yl)-5-(1-hydroxyethyl)-[2,3′-bipyridin]-6′-yl)-2-(5-methylpyridin-2-yl)cyclopropane-1-carboxamide (mixture of two diastereomers). To a stirred solution of HCl in 1,4-dioxane (1.2 mL, 4.0 M) and DCM (5.0 mL), tert-butyl 3-cyano-1-(5-(1-hydroxyethyl)-6′-((1S,2R)-2-(5-methylpyridin-2-yl) cyclopropane-1-carboxamido)-[2,3′-bipyridin]-6-yl)-1,4,6,7-tetrahydro-5H-pyrazolo[4,3-c]pyridine-5-carboxylate (150 mg, 0.242 mmol) was added at 0° C. under nitrogen atmosphere. The resulting mixture was stirred at for 1.0 h and concentrated under reduced pressure.

The residue was purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient (B %): 24% B to 44% B in 10 min; Wave Length: 254 nm/220 nm; RT1(min): 8.36) to afford the title compound (65 mg, 52%) as a white solid. LCMS: 521 [M+H]⁺.

Step 8. Synthesis of (1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 1) and (1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 2). (1S,2R)—N-(6-(3-cyano-4,5,6,7-tetrahydro-1H-pyrazolo[4,3-c]pyridin-1-yl)-5-(1-hydroxyethyl)-[2,3′-bipyridin]-6′-yl)-2-(5-methylpyridin-2-yl)cyclopropane-1-carboxamide (mixture of two diastereomers) (30 mg, 97.7% purity) was separated by Chiral-HPLC with the following conditions: Column: JW-CHIRALPAK IG, 20×250 mm, 5 μm; Mobile Phase A: EtOH:DCM=1:1-HPLC, Mobile Phase B: Hex (0.5% 2M NH₃-MeOH)—HPLC; Flow rate: 20 mL/min; Gradient (B %): 50% B to 50% B in 21 min; Wave Length: 220/254 nm; RT1 (min): 13.12; RT2 (min): 18.08; Sample Solvent: EtOH:DCM=1:1-HPLC. The desired fractions were combined, concentrated, and lyophilized to provide the title compound, isomer 1 (I-21, 10.9 mg) as a white solid and the title compound: isomer 2 (I-67, 11.1 mg) as a white solid.

(1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 1). LCMS: (Method E) Rt=1.154 mins (99.3% purity), ESI-MS m/z 521 (M+H). Chiral HPLC: Rt=2.824 mins (ee=100%) (Column: CHIRALPAK IG-3; Mobile Phase A: Hex (0.1% DEA): (EtOH:DCM=1:1)=50:50; Flow rate: 1 mL/min). ¹H NMR (400 MHz, DMSO-d6) δ (ppm): 10.94 (1H), 8.97 (1H), 8.33-8.30 (2H), 8.29-8.19 (2H), 7.93 (1H), 7.52-7.37 (1H), 7.19 (1H), 5.43 (1H), 4.90-4.74 (1H), 3.86 (2H), 3.03-2.86 (2H), 2.77-2.60 (3H), 2.48 (1H), 2.21 (3H), 1.77-1.70 (1H), 1.43-1.35 (1H), 1.25 (3H).

(1S,2R)—N-(6-(3-cyano-4,5,6,7-tetrahydro-1H-pyrazolo[4,3-c]pyridin-1-yl)-5-((R*)-1-hydroxyethyl)-[2,3′-bipyridin]-6′-yl)-2-(5-methylpyridin-2-yl) cyclopropane-1-carboxamide (Isomer 2). LCMS: (Method E) Rt=0.838 mins (99.6% purity), ESI-MS m/z 521 (M+H). Chiral HPLC: Rt=3.863 mins (ee=100%) (Column: CHIRALPAK IG-3; Mobile Phase A: Hex (0.1% DEA): (EtOH:DCM=1:1)=50:50; Flow rate: 1 mL/min). ¹H NMR (400 MHz, DMSO-d6) δ (ppm): 10.94 (1H), 8.97 (1H), 8.35-8.27 (2H), 8.24-8.18 (2H), 7.93 (1H), 7.49-7.42 (1H), 7.19 (1H), 5.42 (1H), 4.88-4.76 (1H), 3.85 (2H), 3.02-2.86 (2H), 2.76-2.60 (3H), 2.47 (1H), 2.22 (3H), 1.80-1.67 (1H), 1.46-1.36 (1H), 1.26 (3H).

### Example 15. Synthesis of (1S,2R)—N-(6-(3-cyano-5-methyl-1H-pyrazol-1-yl)-5-((S)-1-hydroxyethyl)-[2,3′-bipyridin]-6′-yl)-2-(5-(difluoromethyl)pyrimidin-2-yl)cyclopropane-1-carboxamide (I-51)

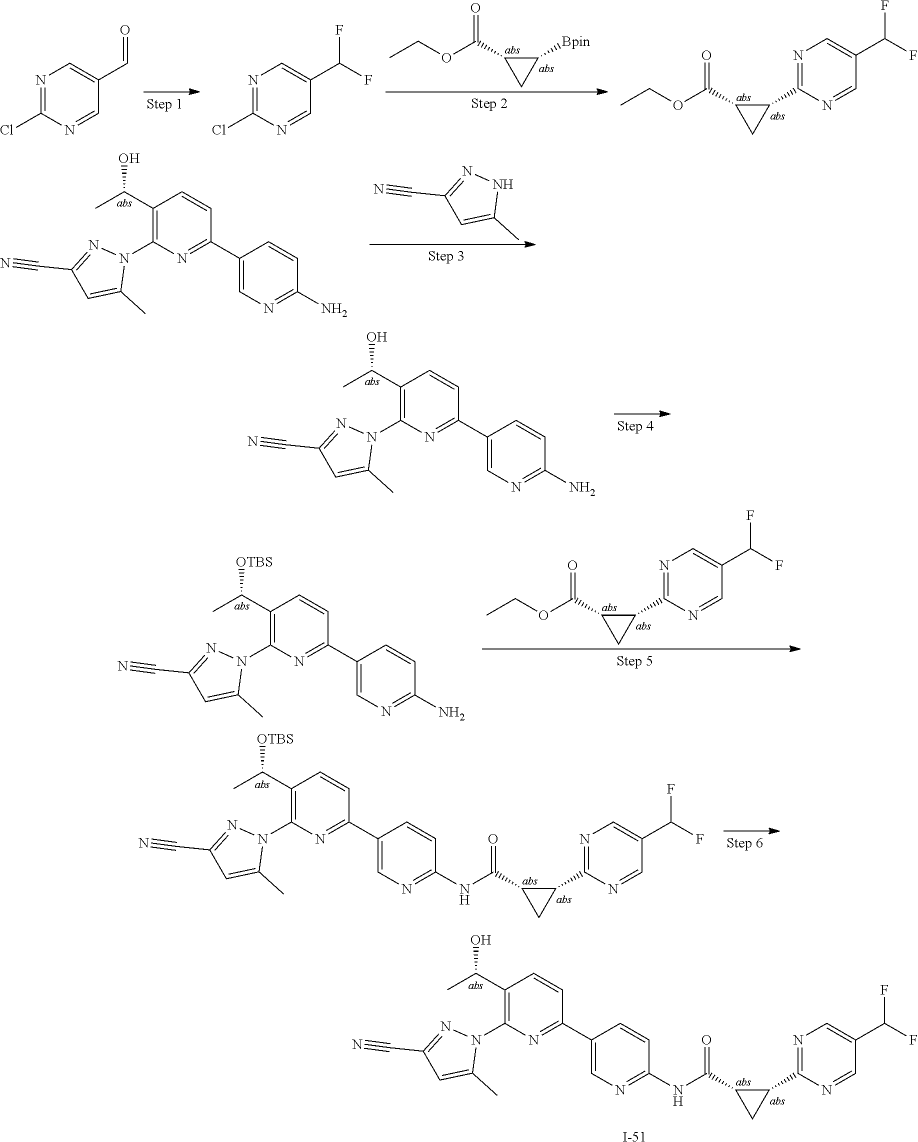

Step 1. Synthesis of 2-chloro-5-(difluoromethyl)pyrimidine. To a solution of 2-chloropyrimidine-5-carbaldehyde (10.00 g, 70.15 mmol, 1 eq) in DCM (200 mL) was added DAST (24.88 g, 154.34 mmol, 20.39 mL, 2.2 eq) at 0° C. under N₂ atmosphere. The mixture was stirred at 0° C. for 1 h then warmed to 25° C. and stirred for 1 h. The mixture was diluted with saturated aqueous NH₄Cl (1L) and extracted with DCM (3×200 mL), the combined organic layer was washed with brine (1L), dried over anhydrous Na₂SO₄, filtered and concentrated under reduced pressure. The residue was purified by flash silica gel chromatography (Eluent of PE/EtOAc) to afford the title compound. LCMS: 165.2 [M+H]⁺.

Step 2. Synthesis of ethyl (1S,2R)-2-(5-(difluoromethyl)pyrimidin-2-yl)cyclopropane-1-carboxylate. To a solution of 2-chloro-5-(difluoromethyl)pyrimidine (3.80 g, 23.09 mmol, 1 eq) and ethyl (1S,2R)-2-(4,4,5,5-tetramethyl-1,3,2-dioxaborolan-2-yl)cyclopropanecarboxylate (8.32 g, 34.64 mmol, 1.5 eq) in dioxane (100 mL) and H₂O (10 mL) was added cataCXium® A (1.66 g, 4.62 mmol, 0.2 eq), Cs₂CO₃ (15.05 g, 46.19 mmol, 2 eq) and Pd(OAc)₂ (1.04 g, 4.62 mmol, 0.2 eq) under N₂. The mixture was stirred at 100° C. for 16 h. The reaction mixture was filtered and washed with 40 mL EtOAc. The filtrate was concentrated under reduced pressure to give a crude product. The crude product was purified by reverse phase preparative HPLC (MeCN/H₂O, 0.1% FA) and flash silica gel chromatography (Eluent of DCM/MeOH) to afford the title compound. ¹H NMR (400 MHz, DMSO-d6) δ (ppm): 8.91 (2H), 7.18 (1H), 3.91-3.75 (2H), 2.87 (1H), 2.36-2.19 (1H), 1.85-1.70 (1H), 1.55-1.38 (1H), 0.93 (3H). LCMS: 243.0 [M+H]⁺.

Step 3. Synthesis of (S)-1-(6′-amino-5-(1-hydroxyethyl)-[2,3′-bipyridin]-6-yl)-5-methyl-1H-pyrazole-3-carbonitrile. To a solution of (1S)-1-[6-(6-amino-3-pyridyl)-2-fluoro-3-pyridyl]ethanol (2.50 g, 10.72 mmol, 1 eq) and 5-methyl-1H-pyrazole-3-carbonitrile (5.74 g, 53.59 mmol, 5 eq) in NMP (50 mL) was added Cs₂CO₃ (17.46 g, 53.59 mmol, 5 eq). The mixture was stirred at 140° C. for 3 h. The reaction mixture was filtered and concentrated under reduced pressure to give a residue. The residue was purified by reverse phase preparative HPLC (ACN/H₂O, 0.05% NH₄OH) to afford the title compound. ¹H NMR (400 MHz, DMSO-d6) δ (ppm): 8.66 (1H), 8.19 (1H), 8.11-7.98 (2H), 7.03 (1H), 6.52 (1H), 6.41 (2H), 5.57-5.26 (1H), 4.52 (1H), 2.29 (3H), 1.25-1.18 (3H). LCMS: 321.2 [M+H]⁺.

Step 4. Synthesis of (S)-1-(6′-amino-5-(1-((tert-butyldimethylsilyl)oxy)ethyl)-[2,3′-bipyridin]-6-yl)-5-methyl-1H-pyrazole-3-carbonitrile. To a solution of 1-[6-(6-amino-3-pyridyl)-3-[(1S)-1-hydroxyethyl]-2-pyridyl]-5-methyl-pyrazole-3-carbonitrile (520 mg, 1.62 mmol, 1 eq) in DMF (10 mL) was added TBSCl (734 mg, 4.87 mmol, 3 eq) and imidazole (442 mg, 6.49 mmol, 4 eq). The mixture was stirred at 25° C. for 1 h. The mixture was quenched by saturated aqueous NaHCO₃ (10 mL) and extracted with EtOAc (3×10 mL), the combined organic layer was washed with brine (10 ml), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, DMSO-d6) δ (ppm): 8.67 (1H), 8.13 (2H), 8.08-8.02 (1H), 7.07 (1H), 6.52 (1H), 6.42 (2H), 4.76 (1H), 2.32 (3H), 1.27 (3H), 0.81 (9H), 0.01-0.07 (3H), −0.13 (3H). LCMS: 435.3 [M+H]⁺.

Step 5. Synthesis of (1S,2R)—N-(5-((S)-1-((tert-butyldimethylsilyl)oxy)ethyl)-6-(3-cyano-5-methyl-1H-pyrazol-1-yl)-[2,3′-bipyridin]-6′-yl)-2-(5-(difluoromethyl)pyrimidin-2-yl) cyclopropane-1-carboxamide. A mixture of 1-[6-(6-amino-3-pyridyl)-3-[(1S)-1-[tert-butyl(dimethyl)silyl]oxyethyl]-2-pyridyl]-5-methyl-pyrazole-3-carbonitrile (150 mg, 0.34 mmol, 1 eq) and ethyl (1S,2R)-2-[5-(difluoromethyl) pyrimidin-2-yl]cyclopropanecarboxylate (125 mg, 0.52 mmol, 1.5 eq) in dioxane (3 mL) was degassed and purged with N₂ for 3 times, and then LiHMDS (1 M in THF, 0.69 mL, 2 eq) was added to the solution at 40° C. under N₂ atmosphere. The mixture was stirred at 40° C. for 1 h under N₂ atmosphere. The reaction mixture was added with saturated aqueous NH₄Cl (5 mL) and extracted with EtOAc (10 mL×3). The combined organic layer was washed with brine (10 mL×2), dried over anhydrous Na₂SO₄, filtered and concentrated under reduced pressure to give a residue. The residue was purified by flash silica gel chromatography (PE/EtOAc & DCM/MeOH) to afford the title compound. LCMS: 631.3 [M+H]⁺.

Step 6. Synthesis of (1S,2R)—N-(6-(3-cyano-5-methyl-1H-pyrazol-1-yl)-5-((S)-1-hydroxyethyl)-[2,3′-bipyridin]-6′-yl)-2-(5-(difluoromethyl)pyrimidin-2-yl)cyclopropane-1-carboxamide (I-51). To a solution of (1S,2R)—N-(5-((S)-1-((tert-butyldimethylsilyl) oxy) ethyl)-6-(3-cyano-5-methyl-1H-pyrazol-1-yl)-[2,3′-bipyridin]-6′-yl)-2-(5-(difluoromethyl)pyrimidin-2-yl) cyclopropane-1-carboxamide (100 mg, 0.16 mmol, 1 eq) in THF (4 mL) was added TBAF (1 M in THF, 0.16 mL, 1 eq). The mixture was stirred at 25° C. for 1 h. The reaction mixture was concentrated under reduced pressure to give the residue. The residue was purified by flash silica gel chromatography (Eluent of DCM/MeOH) and reverse phase preparative HPLC (ACN/H₂O, 0.225% FA) to afford the title compound. ¹H NMR (400 MHz, CD₃OD) δ (ppm): 8.95 (1H), 8.82 (2H), 8.36-8.27 (2H), 8.12 (1H), 7.95 (1H), 7.08-6.76 (2H), 4.78-4.68 (1H), 3.01-2.87 (1H), 2.62-2.52 (1H), 2.36 (3H), 2.13-2.04 (1H), 1.64-1.55 (1H), 1.36 (3H). LCMS: 517.1 [M+H]⁺.

### Example 16. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 1) & (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 2)

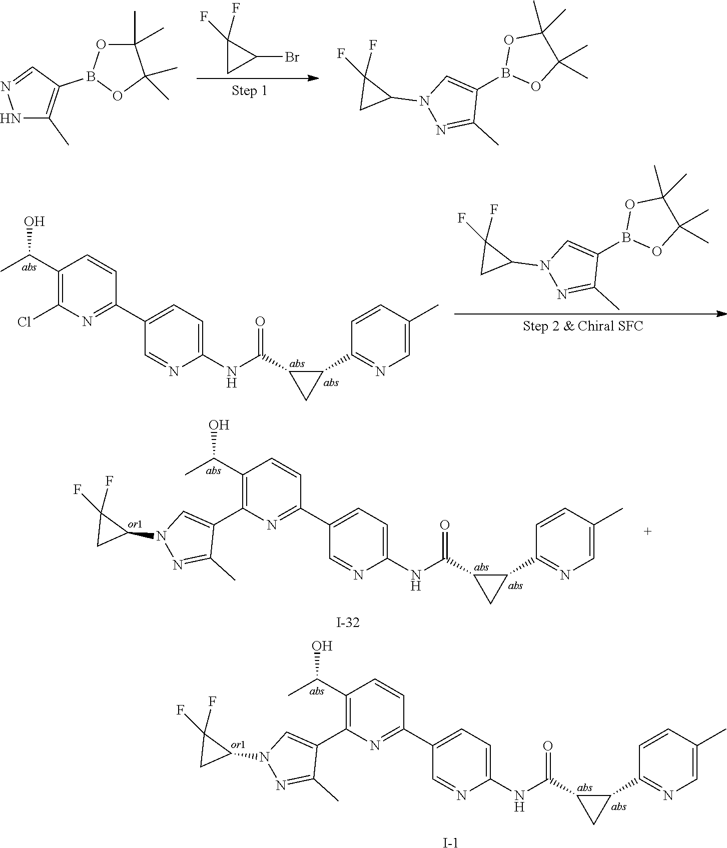

Step 1. Synthesis of 1-(2,2-difluorocyclopropyl)-3-methyl-4-(4,4,5,5-tetramethyl-1,3,2-dioxaborolan-2-yl)-1H-pyrazole. A mixture of 5-methyl-4-(4,4,5,5-tetramethyl-1,3,2-dioxaborolan-2-yl)-1H-pyrazole (600 mg, 2.88 mmol, 1 eq), 2-bromo-1,1-difluoro-cyclopropane (815 mg, 5.19 mmol, 1.8 eq) and Cs₂CO₃ (1.88 g, 5.77 mmol, 2 eq) in DMF (12 mL) was purged with N₂ and subsequently stirred at 120° C. for 16 h. The reaction mixture was diluted with H₂O (50 mL) at 20° C. and extracted with EtOAc (50 mL×3). The combined organic layer was washed with brine (50 mL), dried over anhydrous Na₂SO₄, filtered and concentrated under reduced pressure to give a residue. The residue was purified by flash silica gel chromatography (Eluent of EtOAC/PE) and reverse phase preparative HPLC (ACN/H₂O, 0.225% FA) to afford the title compound as a single regioisomer. ¹H NMR (400 MHz, CD₃OD) δ (ppm): 7.79 (1H), 4.29-4.15 (1H), 2.32 (3H), 2.25-2.13 (2H), 1.31 (12H). LCMS: 285.3 [M+H]⁺.

Step 2. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 1) & (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 2). To a solution of (1S,2R)—N-[5-[6-chloro-5-[(1S)-1-hydroxyethyl]-2-pyridyl]-2-pyridyl]-2-(5-methyl-2-pyridyl)cyclopropanecarboxamide (50 mg, 122 mol, 1 eq) in dioxane (1 mL) and H₂O (0.2 mL) was added 1-(2,2-difluorocyclopropyl)-3-methyl-4-(4,4,5,5-tetramethyl-1,3,2-dioxaborolan-2-yl)pyrazole (69 mg, 245 mol, 2 eq), K₃PO₄ (65 mg, 306 mol, 2.5 eq), and Pd(dppf)Cl₂·CH₂Cl₂ (10. mg, 12 mol, 0.1 eq). The mixture was stirred at 80° C. for 16 h under N₂. The solvent was evaporated. The residue was purified by flash silica gel chromatography (Eluent of MeOH/DCM) to give the mixture of two isomers. The two isomers were separated by chiral SFC (SFC Preparative Method: Instrument: Waters 150 Preparative SFC system; Column: Daicel Chiralpak IG 250×30 mm I.D., 10 m particle size; Mobilephase A: CO₂, Mobile Phase B: MeOH (0.1% NH₄OH); Isocratic elution: 45% Phase B in Supercritical CO₂; Flow rate: 150 g/min; Retention time: Peak1: 6.30 min, Peak2: 7.83 min; Back Pressure: 80 bar to keep the CO₂ in Supercritical flow; Wave Length: 220 nm) to afford isomer 1 (I-32) and isomer 2 (I-1).

(1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 1). ¹H NMR (400 MHz, CD₃OD) δ (ppm): 8.90 (1H), 8.27-8.24 (1H), 8.23 (1H), 8.10 (1H), 7.91-7.87 (2H), 7.82 (1H), 7.56-7.52 (1H), 7.29 (1H), 4.96-4.91 (1H), 4.37-4.29 (1H), 2.77-2.67 (1H), 2.49-2.40 (1H), 2.37-2.31 (1H), 2.30 (3H), 2.29 (3H), 2.27-2.19 (1H), 1.90-1.85 (1H), 1.52-1.46 (1H), 1.41 (3H). LCMS: 531.3 [M+H]⁺. Chiral SFC: Rt=1.955 min (instrument: Shimadzu LC-30AD; column: chiralpak IG-3 50×4.6 mm I.D., particle size 3 μM; mobile phase A: CO₂, mobile phase B: MeOH (0.05% DEA); 3.0 mL/min; 35° C.; 10 MPa, 220 nm)

(1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-methylpyridin-2-yl)cyclopropane-1-carboxamide (Isomer 2). ¹H NMR (400 MHz, CD₃OD) δ (ppm): 8.90 (1H), 8.27-8.24 (1H), 8.23 (1H), 8.10 (1H), 7.91-7.86 (2H), 7.82 (1H), 7.57-7.51 (1H), 7.29 (1H), 4.99-4.91 (1H), 4.39-4.26 (1H), 2.76-2.68 (1H), 2.47-2.41 (1H), 2.33 (1H), 2.30 (3H), 2.29 (3H), 2.27-2.19 (1H), 1.90-1.84 (1H), 1.52-1.45 (1H), 1.40 (3H). LCMS: 531.3 [M+H]⁺. SFC: Rt=2.368 min (instrument: Shimadzu LC-30AD; column: chiralpak IG-3 50×4.6 mm I.D., particle size 3 μM; mobile phase A: CO₂, mobile phase B: MeOH (0.05% DEA); 3.0 mL/min; 35° C.; 10 MPa, 220 nm).

### Example 17. Synthesis of (1S,2R)—N-(6-(3-cyano-5-methyl-1H-pyrazol-1-yl)-5-((S)-1-hydroxyethyl)-[2,3′-bipyridin]-6′-yl)-2-(5-(difluoromethyl)pyridin-2-yl)cyclopropane-1-carboxamide (I-11)

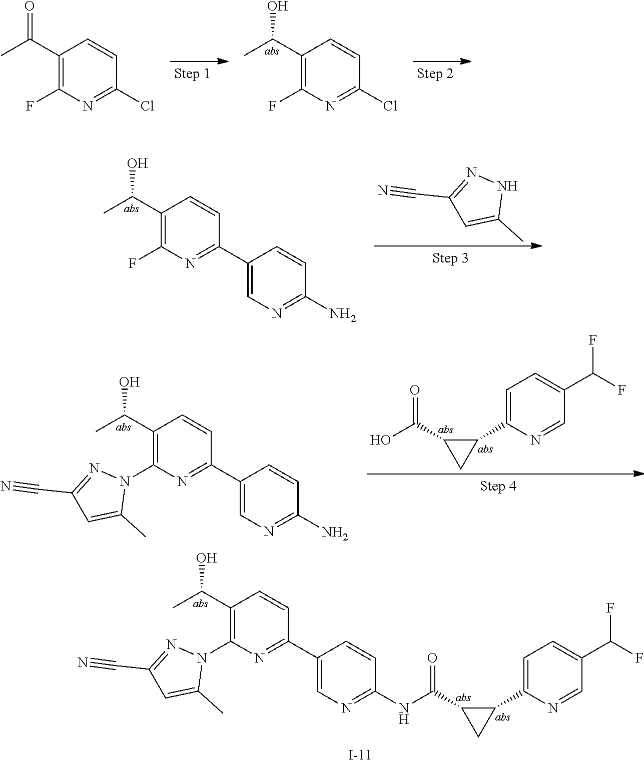

Step 1. Synthesis of (S)-1-(6-chloro-2-fluoropyridin-3-yl) ethan-1-ol. To a solution of 1-(6-chloro-2-fluoropyridin-3-yl)ethan-1-one (10.00 g, 57.6 mmol, 1 eq) in DMSO (250 mL) and H₂O (2.5 L) was added Tris-buffer (1 M, 100 mL, 1.74 eq) and purple carrot cubes (2.00 kg) and the mixture was stirred at 25° C. for 16 h. The reaction solution was filtered, and the filtrate was diluted with water (5 L) and extracted with EtOAc (1L×3). The combined organic layers were washed with brine (1 L×2), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, DMSO-d6) δ (ppm): 8.14-7.95 (1H), 7.57-7.44 (1H), 5.57 (1H), 4.94-4.80 (1H), 1.33 (3H). LCMS: 176.2 [M+H]⁺. Chiral-SFC: Rt=0.470, ee value=100%.

Step 2. Synthesis of (S)-1-(6′-amino-6-fluoro-[2, 3′-bipyridin]-5-yl) ethan-1-ol. To a solution of (S)-1-(6-chloro-2-fluoropyridin-3-yl)ethan-1-ol (56.00 g, 318.93 mmol, 1 eq) and 5-(4,4,5,5-tetramethyl-1,3,2-dioxaborolan-2-yl)pyridin-2-amine (84.00 g, 382.71 mmol, 1.2 eq) in dioxane (600 mL) and H₂O (120 mL) was added K₃PO₄ (169 g, 797.32 mmol, 2.5 eq) and Pd(dppf)Cl₂·CH₂Cl₂ (26.0 g, 31.89 mmol, 0.1 eq). The mixture was stirred at 80° C. for 16 h under N₂. The reaction solution was filtered, and the filtrate was concentrated under reduced pressure to give a residue. The residue was purified by flash silica gel chromatography (Eluent of PE/EtOAc) to afford the title compound. ¹H NMR (400 MHz, DMSO-d6) δ (ppm): 8.62 (1H), 8.05-7.89 (2H), 7.82-7.67 (1H), 6.51 (1H), 6.36 (2H), 5.43 (1H), 5.02-4.81 (1H), 1.35 (3H). LCMS: 234.3 [M+H]⁺.

Step 3. Synthesis of (S)-1-(6′-amino-5-(1-hydroxyethyl)-[2,3′-bipyridin]-6-yl)-5-methyl-1H-pyrazole-3-carbonitrile. To a solution of (1S)-1-[6-(6-amino-3-pyridyl)-2-fluoro-3-pyridyl]ethanol (2.50 g, 10.72 mmol, 1 eq) and 5-methyl-1H-pyrazole-3-carbonitrile (5.74 g, 53.59 mmol, 5 eq) in NMP (50 mL) was added Cs₂CO₃ (17.46 g, 53.59 mmol, 5 eq). The mixture was stirred at 140° C. for 3 h. The reaction mixture was filtered and concentrated under reduced pressure to give a residue. The residue was purified by reverse phase preparative HPLC (ACN/H₂O, 0.05% NH₄OH) to afford the title compound. ¹H NMR (400 MHz, DMSO-d6) δ (ppm): 8.66 (1H), 8.19 (1H), 8.11-7.98 (2H), 7.03 (1H), 6.52 (1H), 6.41 (2H), 5.57-5.26 (1H), 4.52 (1H), 2.29 (3H), 1.25-1.18 (3H). LCMS: 321.2 [M+H]⁺.

Step 4. Synthesis of (1S,2R)—N-(6-(3-cyano-5-methyl-1H-pyrazol-1-yl)-5-((S)-1-hydroxyethyl)-[2,3′-bipyridin]-6′-yl)-2-(5-(difluoromethyl)pyridin-2-yl)cyclopropane-1-carboxamide (I-11). To a solution of 1-[6-(6-amino-3-pyridyl)-3-[(1S)-1-hydroxyethyl]-2-pyridyl]-5-methyl-pyrazole-3-carbonitrile (100 mg, 312 mol, 1 eq) and (1S,2R)-2-[5-(difluoromethyl)-2-pyridyl]cyclopropanecarboxylic acid (133 mg, 624 mol, 2 eq) in Pyridine (1 mL) was added HATU (237 mg, 624 mol, 2 eq). The mixture was stirred at 25° C. for 1 h. The mixture was diluted with H₂O (10 mL) and extracted with EtOAc (3×20 mL), the combined organic layer was washed with brine (10 ml), dried over anhydrous Na₂SO₄, filtered and concentrated under reduced pressure to obtained a residue. The residue was purified by reverse phase preparative HPLC (ACN/H₂O, 0.05% NH₄OH) to afford the title compound. ¹H NMR (400 MHz, CDCl₃) δ (ppm): 9.42-9.24 (1H), 8.87 (1H), 8.68 (1H), 8.17 (2H), 8.11-8.05 (1H), 7.80 (2H), 7.47 (1H), 6.84-6.49 (2H), 4.87-4.76 (1H), 3.75-3.44 (1H), 2.86-2.73 (1H), 2.48 (3H), 2.39-2.30 (1H), 2.13-2.02 (1H), 1.66-1.61 (1H), 1.47 (3H). LCMS: 516.3 [M+H]⁺.

### Example 18. Synthesis of (1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1) & (1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2)

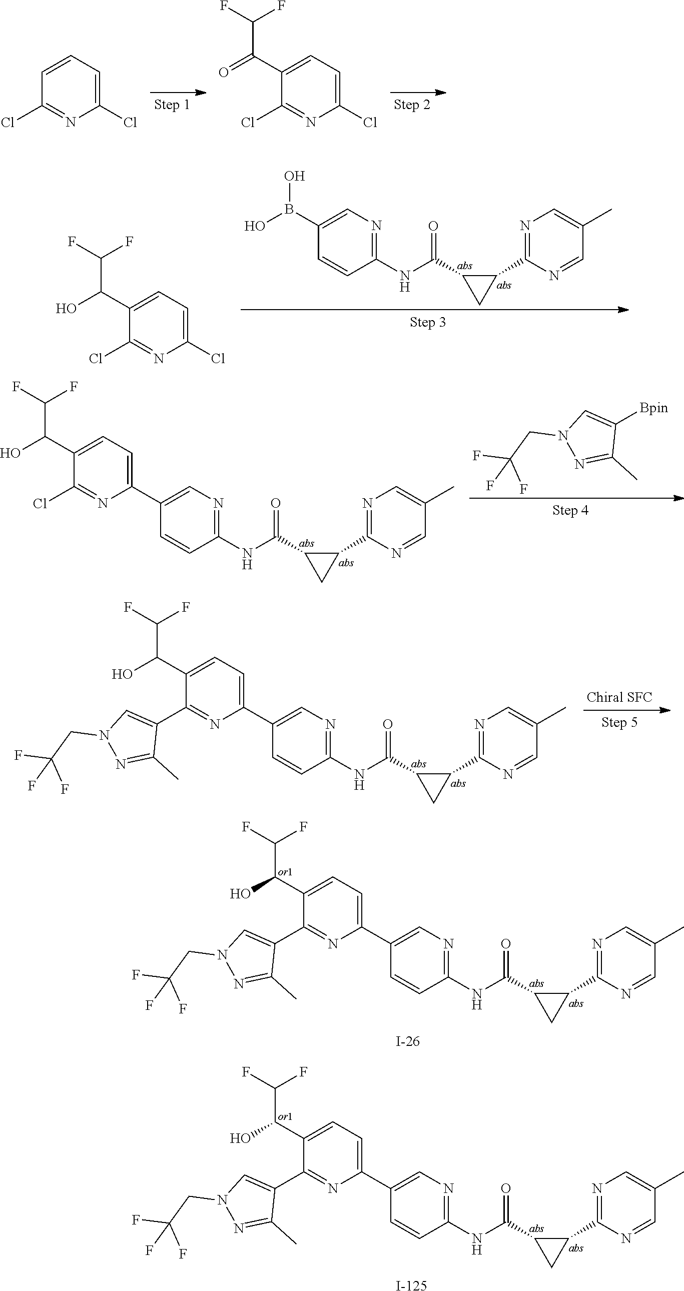

Step 1. Synthesis of 1-(2, 6-dichloropyridin-3-yl)-2, 2-difluoroethan-1-one. To a solution of 2,6-dichloropyridine (10.00 g, 67.6 mmol, 1 eq) in THF (100 mL) was added LDA (2 M in THF/hexane, 43.9 mL, 1.3 eq) at −78° C. under N₂ and the mixture was stirred for 1 h. 2, 2-difluoro-N-methoxy-N-methyl-acetamide (11.30 g, 81.1 mmol, 1.2 eq) was added at −78° C. and the mixture was stirred for 1 h, then the mixture was allowed to warm to 25° C. and stirred 16 h at RT. The mixture was quenched by saturated aqueous NH₄Cl (200 mL), and extracted with EtOAc (3×50 mL), the combined organic layer was washed with brine (100 ml), dried over anhydrous Na₂SO₄, filtered and concentrated under reduced pressure. The residue was purified by flash silica gel chromatography (Eluent of PE/EtOAc) to afford the title compound. ¹H NMR (400 MHz, CDCl₃) δ (ppm): 8.04 (1H), 7.45 (1H), 6.61-6.25 (1H).

Step 2. Synthesis of 1-(2, 6-dichloropyridin-3-yl)-2, 2-difluoroethan-1-ol. To a solution of 1-(2, 6-dichloropyridin-3-yl)-2, 2-difluoroethan-1-one (1.00 g, 4.42 mmol, 1 eq) in THF (10 mL) was added NaBH₄ (502 mg, 13.27 mmol, 3 eq) under N₂ at 0° C. The mixture was stirred at 0° C. for 1 h. The mixture was quenched by saturated aqueous NH₄Cl (10 mL) and extracted with EtOAc (3×20 mL), the combined organic layer was washed with brine (20 ml), dried over anhydrous Na₂SO₄, filtered and concentrated under reduced pressure to give a residue. The residue was purified by reverse phase preparative HPLC (ACN/H₂O, 0.225% FA) to afford the title compound. ¹H NMR (400 MHz, CDCl₃) δ (ppm): 7.98 (1H), 7.38 (1H), 6.13-5.75 (1H), 5.43-5.24 (1H), 3.27 (1H). LCMS: 228.1 [M+H]⁺.

Step 3. Synthesis of (1S, 2R)—N-(6-chloro-5-(2, 2-difluoro-1-hydroxyethyl)-[2, 3′-bipyridin]-6′-yl)-2-(5-methylpyrimidin-2-yl) cyclopropane-1-carboxamide. To a solution of 1-(2,6-dichloropyridin-3-yl)-2,2-difluoroethan-1-ol (150 mg, 658 mol, 1 eq) and (6-((1S,2R)-2-(5-methylpyrimidin-2-yl)cyclopropane-1-carboxamido)pyridin-3-yl)boronic acid (176 mg, 592 mol, 0.9 eq) in dioxane (1.5 mL) and H₂O (0.3 mL) was added Pd(dppf)Cl₂·CH₂Cl₂ (54 mg, 66 mol, 0.1 eq) and K₃PO₄ (279 mg, 1.32 mmol, 2 eq). The mixture was stirred at 40° C. for 1 h. The mixture was diluted with 5 mL dioxane and filtered through Celite. The filtrate was concentrated in vacuum to give a residue. The residue was purified by flash silica gel chromatography (Eluent of DCM/MeOH) to afford the title compound. ¹H NMR (400 MHz, CDCl₃) δ (ppm): 9.45 (1H), 8.95-8.80 (1H), 8.52 (2H), 8.24-8.16 (1H), 8.15-8.10 (1H), 8.05-8.00 (1H), 7.70-7.62 (1H), 6.00 (1H), 5.50-5.23 (1H), 3.27-3.08 (1H), 2.92-2.80 (1H), 2.37-2.30 (1H), 2.29-2.26 (3H), 2.14-2.08 (1H), 1.71-1.64 (1H). LCMS: 446.2 [M+H]⁺.

Step 4. Synthesis of (1S, 2R)—N-(5-(2, 2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide. To a solution of (1S,2R)—N-(6-chloro-5-(2,2-difluoro-1-hydroxyethyl)-[2,3′-bipyridin]-6′-yl)-2-(5-methylpyrimidin-2-yl)cyclopropane-1-carboxamide (240 mg, 538 mol, 1 eq) and 3-methyl-4-(4,4,5,5-tetramethyl-1,3,2-dioxaborolan-2-yl)-1-(2,2,2-trifluoroethyl)pyrazole (468 mg, 1.61 mmol, 3 eq) in dioxane (2.4 mL) and H₂O (0.6 mL) was added K₃PO₄ (343 mg, 1.61 mmol, 3 eq) and Pd(dppf)Cl₂·CH₂Cl₂ (88 mg, 108 mol, 0.2 eq) under N₂. The mixture was stirred at 80° C. for 1 h. The mixture was diluted with H₂O (10 mL), and extracted with EtOAc (3×20 mL), the combined organic layer was washed with brine (10 ml), dried over anhydrous Na₂SO₄, filtered and concentrated under reduced pressure to give a residue. The residue was purified flash silica gel chromatography (Eluent of DCM/MeOH) and reverse phase preparative HPLC (ACN/H₂O, 0.05% NH₄OH) to afford the title compound. ¹H NMR (400 MHz, CD₃OD) δ (ppm): 8.98-8.92 (1H), 8.51 (2H), 8.35-8.28 (1H), 8.15-8.08 (1H), 7.93 (1H), 7.87 (2H), 6.16-5.75 (1H), 4.99-4.91 (3H), 2.88-2.79 (1H), 2.53-2.44 (1H), 2.29 (6H), 2.06-1.98 (1H), 1.56-1.49 (1H). LCMS: 574.3 [M+H]⁺.

Step 5. Synthesis of (1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1) & (1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2). (1S, 2R)—N-(5-(2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide was separated by chiral SFC (SFC Preparative Method: Instrument: Waters 80Q Preparative SFC system; Column: DAICEL Chiralpak AD 250×25 mm I.D., 10 m particle size; Mobilephase A: CO₂, Mobile Phase B: EtOH (0.2% NH₄OH); Isocratic elution: 55% Phase B in Supercritical CO₂; Flow rate: 75g/min; Retention time: Peak1: 5.53 min, Peak2: 7.32 min; Back Pressure: 100 bar to keep the CO₂ in Supercritical flow; Wave Length: 220 nm) to give isomer 1 (I-26) and peak 2 (I-125).

(1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1). ¹H NMR (400 MHz, CDCl₃) δ (ppm): 9.48 (1H), 8.98-8.86 (1H), 8.50 (2H), 8.28-8.19 (1H), 8.17-8.08 (1H), 8.01 (1H), 7.69 (2H), 5.91 (1H), 5.11-4.99 (1H), 4.78-4.61 (2H), 3.70-3.13 (1H), 2.91-2.77 (1H), 2.39-2.34 (3H), 2.34-2.29 (1H), 2.27 (3H), 2.10 (1H), 1.65 (1H). LCMS: 574.3 [M+H]⁺. Chiral SFC: Rt=1.912 min (instrument: shimadzu LC-30ADsf; column: chiralpak AD-3 50×4.6 mm I.D. particle size 3 μm; mobile phase A: CO₂, mobile phase B: EtOH (0.05% DEA); 3.0 mL/min; 35° C.; 10 MPa; 220 nm).

(1S,2R)—N-(5-((R*)-2,2-difluoro-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2). ¹H NMR (400 MHz, CDCl₃) δ (ppm): 9.41 (1H), 8.90 (1H), 8.49 (2H), 8.28-8.18 (1H), 8.10 (1H), 8.02 (1H), 7.73-7.63 (2H), 6.07-5.73 (1H), 5.11-4.99 (1H), 4.81-4.62 (2H), 3.66-3.15 (1H), 2.86 (1H), 2.35 (3H), 2.33-2.29 (1H), 2.27 (3H), 2.13-2.06 (1H), 1.65 (1H). LCMS: 574.3 [M+H]⁺. Chiral SFC: Rt=2.079 min(instrument: shimadzu LC-30ADsf; column: chiralpak AD-3 50×4.6 mm I.D. particle size 3 μm; mobile phase A: CO₂, mobile phase B: EtOH (0.05% DEA); 3.0 mL/min; 35° C.; 10 MPa; 220 nm).

### Example 19. Synthesis of methyl 6-(3-methyl-1-(2,2,2-trifluoroethyl)-1H-pyrazol-4-yl)-6′-((1S,2R)-2-(5-methylpyridin-2-yl)cyclopropanecarboxamido)-2,3′-bipyridine-5-carboxylate (I-128)

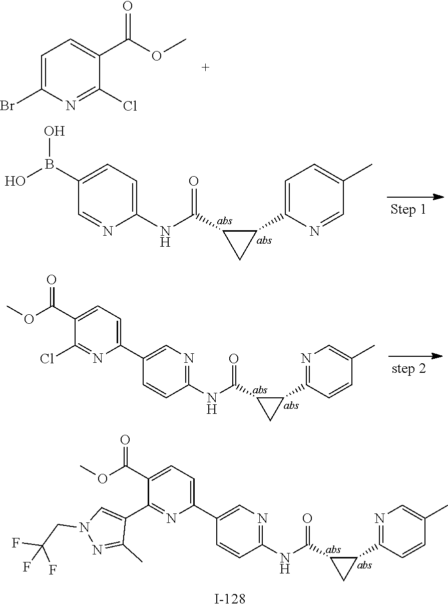

Step 1. Synthesis of methyl 6-chloro-6′-((1S,2R)-2-(5-methylpyridin-2-yl)cyclopropane-1-carboxamido)-[2,3′-bipyridine]-5-carboxylate. To a solution of methyl 6-bromo-2-chloronicotinate (300 mg, 1.2 mmol) and (6-((1S,2R)-2-(5-methylpyridin-2-yl) cyclopropane-1-carboxamido) pyridin-3-yl) boronic acid (427 mg, 1.45 mmol) in dioxane (4.0 mL) and H₂O (0.4 mL) was added Pd(dppf)Cl₂·CH₂Cl₂ (97.8 mg, 0.12 mmol) at RT under N₂ atmosphere. The resulting mixture was stirred at 80° C. overnight and allowed to cool down to RT. The reaction mixture was diluted with water (50 mL) and extracted with EtOAc (3×50 mL). The combined organic layers were dried over Na₂SO₄, filtered, and concentrated under reduced pressure. The residue was purified via reverse flash chromatography (C18 silica gel) using a 10% to 30% ACN in water (with 0.1% NH₄HCO₃) gradient to provide the title compound (480 mg, 95% yield) as a yellow oil. LCMS: 423 [M+H]⁺.

Step 2. Synthesis of methyl 6-(3-methyl-1-(2,2,2-trifluoroethyl)-1H-pyrazol-4-yl)-6′-((1S,2R)-2-(5-methylpyridin-2-yl)cyclopropanecarboxamido)-2,3′-bipyridine-5-carboxylate (I-128). To a solution of methyl 6-chloro-6′-[(1S,2R)-2-(5-methylpyridin-2-yl) cyclopropaneamido]-[2,3′-bipyridine]-5-carboxylate (30 mg, 0.071 mmol) and 3-methyl-4-(4,4,5,5-tetramethyl-1,3,2-dioxaborolan-2-yl)-1-(2,2,2-trifluoroethyl)-1H-pyrazole (31 mg, 0.11 mmol) in 1,4-dioxane (0.8 mL) and H₂O (0.1 mL) were added K₃PO₄ (45.2 mg, 0.213 mmol) and Pd(dppf)Cl₂·CH₂Cl₂ (11.6 mg, 0.014 mmol) at RT. The resulting mixture was stirred at 80° C. for 1 h under N₂ atmosphere.

The mixture was allowed to cool down to RT and concentrated. The residue was purified directly by Prep-HPLC with the following conditions (Column: XSelect CSH Fluoro Phenyl 30×150 mm, 5 m; Mobile Phase A: Water (0.1% FA), Mobile Phase B: ACN; Flow rate: 60 mL/min; Gradient (B %): 24% B to 34% B in 10 min; Wave Length: 254 nm/220 nm; RT1(min): 8.28) to afford the title compound (2.0 mg, 5% yield) as a white solid. LCMS: 551 [M+H]⁺. ¹H NMR (400 MHz, DMSO-d6) δ (ppm): 10.96 (1H), 9.06 (1H), 8.45-8.37 (1H), 8.23 (1H), 8.16 (1H), 8.02-7.91 (3H), 7.49-7.43 (1H), 7.20 (1H), 5.19-5.06 (2H), 3.77 (3H), 2.73-2.56 (2H), 2.32 (3H), 2.22 (3H), 1.78-1.68 (1H), 1.45-1.34 (1H).

### Example 20. Synthesis of (1S,2R)—N-(5-(hydroxymethyl)-6-(3-methyl-1-(2,2,2-trifluoroethyl)-1H-pyrazol-4-yl)-[2,3′-bipyridin]-6′-yl)-2-(5-methylpyridin-2-yl) cyclopropane-1-carboxamide (I-92)

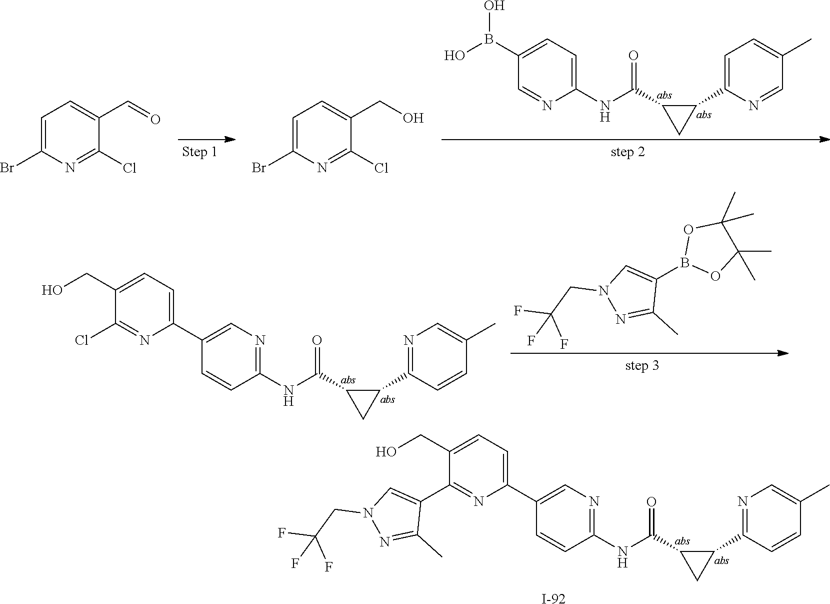

Step 1. Synthesis of (6-bromo-2-chloropyridin-3-yl)methanol. To a solution of 6-bromo-2-chloronicotinaldehyde (300 mg, 1.37 mmol) in THF (5.0 mL) was added DIBAL-H (4.1 mml, 4.1 mL, 1M in THF) at 0° C. under N₂ atmosphere. The resulting mixture was stirred at RT for 3 h and quenched with sat. NH₄Cl (aq.) at 0° C. The resulting mixture was filtered, washed and the filtrate was concentrated under reduced pressure. The residue was purified by Prep-TLC (CH₂Cl₂/MeOH 10:1) to afford the title product (220 mg, 72% yield) as a yellow oil. LCMS: 224 [M+H]⁺.

Step 2. Synthesis of (1S,2R)—N-(6-chloro-5-(hydroxymethyl)-[2,3′-bipyridin]-6′-yl)-2-(5-methylpyridin-2-yl)cyclopropane-1-carboxamide. To a solution of (6-bromo-2-chloropyridin-3-yl)methanol (220 mg, 1.0 mmol) and (6-((1S,2R)-2-(5-methylpyridin-2-yl) cyclopropane-1-carboxamido)pyridin-3-yl)boronic acid (355 mg, 1.19 mmol), K₃PO₄ (623 mg, 3 mmol) in dioxane (6 mL) and H₂O (0.6 mL) were added Pd(dppf)Cl₂·CH₂Cl₂ (81 mg, 0.2 mmol) at RT under N₂ atmosphere. The resulting mixture was stirred at 100° C. for 2 h and allowed to cool down to RT. The reaction mixture was diluted with water (50 mL) and extracted with EtOAc (3×50 mL). The combined organic layers were dried over Na₂SO₄, filtered, and concentrated under reduced pressure. The residue was purified via reverse flash chromatography (C18 silica gel) using a 10% to 30% ACN in water (with 0.1% NH₄HCO₃) gradient to provide the title compound (190 mg, 48% yield) as a yellow solid. LCMS: 395 [M+H]⁺.

Step 3. Synthesis of (1S,2R)—N-(5-(hydroxymethyl)-6-(3-methyl-1-(2,2,2-trifluoroethyl)-1H-pyrazol-4-yl)-[2,3′-bipyridin]-6′-yl)-2-(5-methylpyridin-2-yl) cyclopropane-1-carboxamide (I-92). To a solution of (1S,2R)—N-[6-chloro-5-(hydroxymethyl)-[2,3′-bipyridin]-6′-yl]-2-(5-methylpyridin-2-yl) cyclopropane-1-carboxamide (50 mg, 0.13 mmol), 3-methyl-4-(4,4,5,5-tetramethyl-1,3,2-dioxaborolan-2-yl)-1-(2,2,2trifluoroethyl)pyrazole (55.1 mg, 0.19 mmol) and K₃PO₄ (81 mg, 0.38 mmol) in H₂O (0.2 mL) and 1,4-dioxane (1.6 mL) were added Pd(dppf)Cl₂·CH₂Cl₂ (21 mg, 0.025 mmol) at RT. The resulting mixture was stirred at 80° C. for 1 h under N₂ atmosphere. The mixture was allowed to cool down to RT and concentrated. The residue was directly purified via reverse flash chromatography (C18 silica gel) using a 10% to 50% ACN in water (with 0.1% NH₄HCO₃) gradient to afford the title compound (20.1 mg, 30% yield) as a white solid. LCMS: 523 [M+H]⁺. ¹H NMR (400 MHz, DMSO-d6) δ (ppm): 10.88 (1H), 9.01 (1H), 8.42-8.31 (1H), 8.26-8.16 (2H), 8.01-7.83 (3H), 7.51-7.42 (1H), 7.20 (1H), 5.44 (1H), 5.22-5.08 (2H), 4.51 (2H), 2.73-2.61 (1H), 2.47 (1H), 2.38 (3H), 2.22 (3H), 1.76-1.71 (1H), 1.42-1.36 (1H).

### Example 21. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 1) (I-151) and (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 2) (I-150)

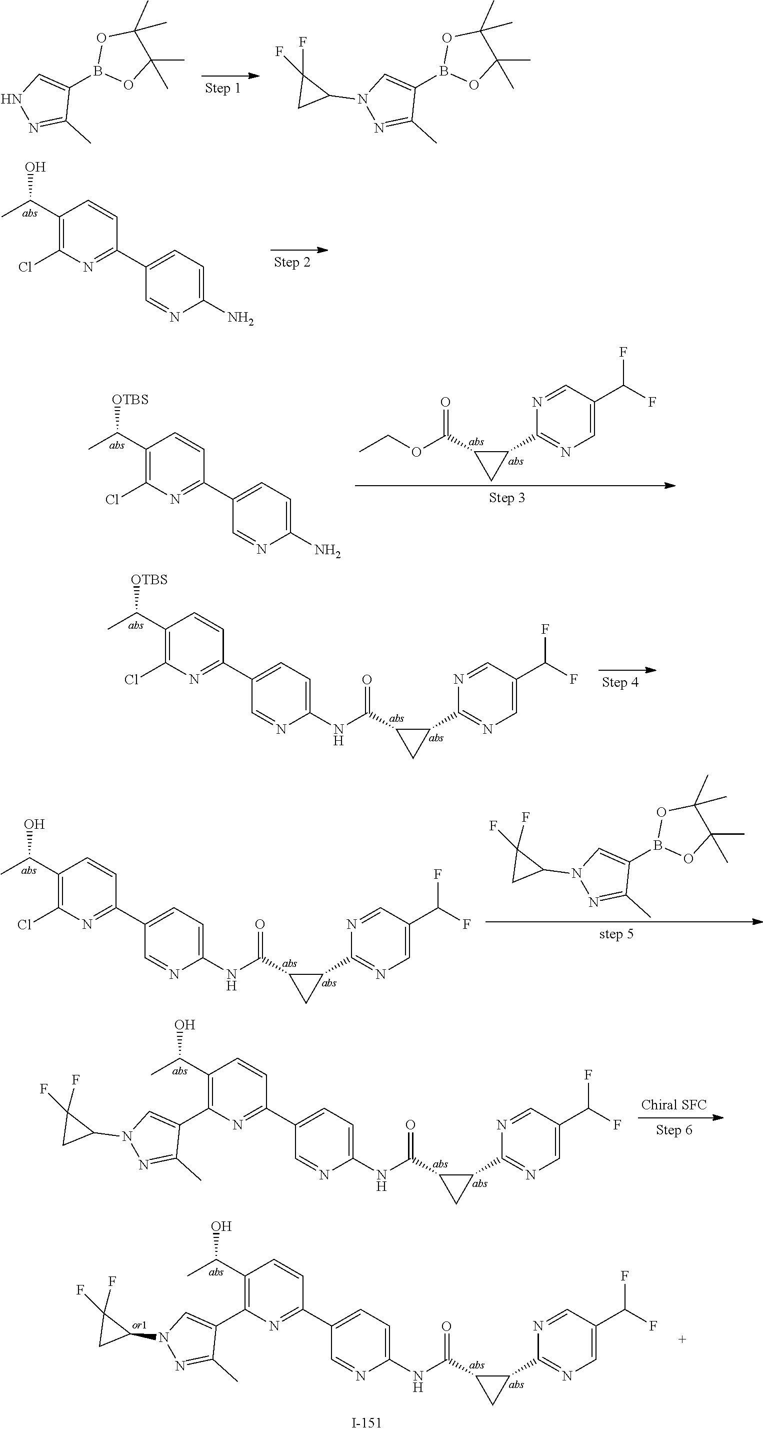

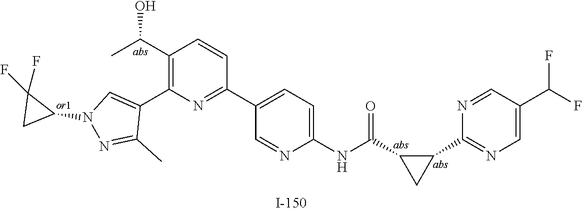 [SMILES: Cc1nn([C@@H]2CC2(F)F)cc1-c1nc(-c2ccc(NC(=O)[C@H]3C[C@H]3c3ncc(C(F)F)cn3)nc2)ccc1[C@H](C)O]

Step 1. Synthesis of 1-(2,2-difluorocyclopropyl)-3-methyl-4-(4,4,5,5-tetramethyl-1,3,2-dioxaborolan-2-yl)-1H-pyrazole. A mixture of 5-methyl-4-(4,4,5,5-tetramethyl-1,3,2-dioxaborolan-2-yl)-1H-pyrazole (600 mg, 2.88 mmol, 1 eq), 2-bromo-1,1-difluoro-cyclopropane (815 mg, 5.19 mmol, 1.8 eq) and Cs₂CO₃ (1.88 g, 5.77 mmol, 2 eq) in DMF (12 mL) was purged with N₂ and subsequently stirred at 120° C. for 16 h. The reaction mixture was diluted with H₂O (50 mL) at 20° C. and extracted with EtOAc (50 mL×3). The combined organic layers were washed with brine (50 mL), dried over anhydrous Na₂SO₄, filtered and concentrated under reduced pressure to give a residue. The residue was purified by flash silica gel chromatography (Eluent of EtOAC/PE) and reverse phase preparative HPLC (ACN/H₂O, 0.225% FA) to afford the title compound. ¹H NMR (400 MHz, CD₃OD) δ (ppm): 7.79 (1H), 4.29-4.15 (1H), 2.32 (3H), 2.25-2.13 (2H), 1.31 (12H). LCMS: 285.3 [M+H]⁺.

Step 2. Synthesis of (S)-5-(1-((tert-butyldimethylsilyl)oxy)ethyl)-6-chloro-[2,3′-bipyridin]-6′-amine. To a solution of (1S)-1-[6-(6-amino-3-pyridyl)-2-chloro-3-pyridyl]ethanol (50 g, 200 mmol, 1 eq) and imidazole (41 g, 600 mmol, 3 eq) in DCM (750 mL) was added a solution of TBSCl (60 g, 400 mmol, 2 eq) in DCM (250 mL) dropwise at 0° C. The mixture was stirred at 60° C. for 0.5 h. The reaction mixture was quenched by H₂O (1 L) at 25° C. and extracted with DCM (400 mL×2). The combined organic layers were washed with brine (500 mL), dried over anhydrous Na₂SO₄, filtered and concentrated under reduced pressure to give a residue. The residue was purified by flash silica gel chromatography (Eluent of EtOAc/PE) to afford the title compound. ¹H NMR (400 MHz, CD₃OD) δ (ppm): 8.58 (1H), 8.14-8.07 (1H), 7.99 (1H), 7.75 (1H), 6.66 (1H), 5.26-5.17 (1H), 1.42 (3H), 0.92 (9H), 0.11 (3H), 0.01 (3H). LCMS: 364.2 [M+H]⁺.

Step 3. Synthesis of (1S,2R)—N-(5-((S)-1-((tert-butyldimethylsilyl)oxy)ethyl)-6-chloro-[2,3′-bipyridin]-6′-yl)-2-(5-(difluoromethyl)pyrimidin-2-yl)cyclopropane-1-carboxamide. To a solution of 5-[5-[(1S)-1-[tert-butyl(dimethyl)silyl]oxyethyl]-6-chloro-2-pyridyl]pyridin-2-amine (48.00 g, 132.11 mmol, 1 eq) and ethyl (1S,2R)-2-[5-(difluoromethyl)pyrimidin-2-yl]cyclopropanecarboxylate (32.00 g, 132.11 mmol, 1 eq) in dioxane (500 mL) was added LiHMDS (1 M in THF, 264 mL, 2 eq) dropwise at 40° C. under N₂ atmosphere. The mixture was stirred at 40° C. for 1 h. The mixture was added into the saturated aqueous NH₄Cl (1 L) solution at 0° C. dropwise under N₂ atmosphere. Then the mixture was extracted with DCM (500 mL×2). The combined organic phase was washed with brine (500 mL), dried with anhydrous Na₂SO₄, filtered and concentrated under reduced pressure to give a residue. The residue was purified through flash silica gel chromatography (Eluent of EtOAc/PE) to afford the title compound. LCMS: 560.2 [M+H]⁺.

Step 4. Synthesis of (1S,2R)—N-(6-chloro-5-((S)-1-hydroxyethyl)-[2,3′-bipyridin]-6′-yl)-2-(5-(difluoromethyl)pyrimidin-2-yl)cyclopropane-1-carboxamide. To a solution of (1S, 2R)—N-[5-[5-[(1S)-1-[tert-butyl(dimethyl)silyl]oxyethyl]-6-chloro-2-pyridyl]-2-pyridyl]-2-[5-(difluoromethyl)pyrimidin-2-yl]cyclopropanecarboxamide (350 mg, 625 mol, 1 eq) in THF (2 mL) was added TBAF (1 M in THF, 1.25 mL, 2 eq). The mixture was stirred at 30° C. for 10 min. The reaction solution was concentrated in vacuum and the residue was purified through flash silica gel chromatography (Eluent of MeOH/DCM) to afford the title compound. ¹H NMR (400 MHz, CD₃OD) δ (ppm): 8.90 (1H), 8.82 (2H), 8.30-8.24 (1H), 8.05 (1H), 7.92 (1H), 7.85 (1H), 6.92 (1H), 5.19-5.11 (1H), 3.02-2.87 (1H), 2.65-2.50 (1H), 2.13-2.05 (1H), 1.63-1.55 (1H), 1.45 (3H). LCMS: 446.0 [M+H]⁺.

Step 5. Synthesis of (1S,2R)—N-(6-(1-(2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide. To a solution of (1S,2R)—N-[5-[6-chloro-5-[(1S)-1-hydroxyethyl]-2-pyridyl]-2-pyridyl]-2-[5-(difluoromethyl)pyrimidin-2-yl]cyclopropanecarboxamide (50 mg, 112 mol, 1 eq) in dioxane (1 mL) and H₂O (0.2 mL) was added 1-(2,2-difluorocyclopropyl)-3-methyl-4-(4,4,5,5-tetramethyl-1,3,2-dioxaborolan-2-yl)pyrazole (64 mg, 224 mol, 2 eq), K₃PO₄ (71 mg, 336 mol, 3 eq) and Pd(dppf)Cl₂·CH₂Cl₂ (9 mg, 11 mol, 0.1 eq). The mixture was stirred at 80° C. for 16 h under N₂ atmosphere. The mixture was diluted with water (5 mL) and extracted with EtOAc (5 mL×3). The combined organic layers were washed with brine (5 mL), dried over anhydrous Na₂SO₄, filtered and concentrated under reduced pressure to give a residue. The residue was purified by reverse phase preparative HPLC (ACN/H₂O, 0.05% NH₄OH) to afford the title compound. LCMS: 568.3 [M+H]⁺.

Step 6. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 1) & (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 2). (1S,2R)—N-(6-(1-(2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide was separated by chiral SFC (SFC Preparative Method: Instrument: Waters 80Q Preparative SFC system; Column: DAICEL Chiralpak AD 250×25 mm I.D., 10 m particle size; Mobilephase A: CO₂, Mobile Phase B: EtOH (0.2% NH₄OH); Isocratic elution: 48% Phase B in Supercritical CO₂; Flow rate: 70 g/min; Retention time: Peak1: 5.65 min, Peak2: 6.68 min; Back Pressure: 100 bar to keep the CO₂ in Supercritical flow; Wave Length: 220 nm) to give the title compound (Isomer 1, I-151) as peak 1 and the title compound (Isomer 2, I-150) as peak 2.

(1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 1). ¹H NMR (400 MHz, CD₃OD) δ (ppm): 8.90 (1H), 8.82 (2H), 8.30-8.26 (1H), 8.10 (1H), 7.92 (1H), 7.90 (1H), 7.83 (1H), 6.92 (1H), 4.96-4.91 (1H), 4.36-4.29 (1H), 2.98-2.90 (1H), 2.61-2.53 (1H), 2.36-2.20 (5H), 2.11-2.06 (1H), 1.62-1.56 (1H), 1.42 (3H). LCMS: 568.2 [M+H]⁺. Chiral SFC: Rt=1.799 min (instrument: SHIMADZU LC-30ADsf; column: Chiralpak AD-3 50×4.6 mm I.D., particle size 3 μm; mobile phase A: CO₂; mobile phase B: EtOH (0.05% DEA); 3.0 mL/min; 35° C.; 10 mPa, 220 nm).

(1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (Isomer 2). ¹H NMR (400 MHz, CD₃OD) δ (ppm): 8.90 (1H), 8.82 (2H), 8.31-8.25 (1H), 8.10 (1H), 7.92 (1H), 7.90 (1H), 7.83 (1H), 6.92 (1H), 4.98-4.93 (1H), 4.38-4.30 (1H), 2.97-2.90 (1H), 2.61-2.54 (1H), 2.34-2.20 (5H), 2.11-2.06 (1H), 1.62-1.56 (1H), 1.40 (3H). LCMS: 568.4 [M+H]⁺. Chiral SFC: Rt=1.954 min (instrument: SHIMADZU LC-30ADsf; column: Chiralpak AD-3 50×4.6 mm I.D., particle size 3 μm; mobile phase A: CO₂; mobile phase B: EtOH (0.05% DEA); 3.0 mL/min; 35° C.; 10 mPa, 220 nm).

### Example 22. Synthesis of (1S,2R)-2-(5-(difluoromethyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (I-47)

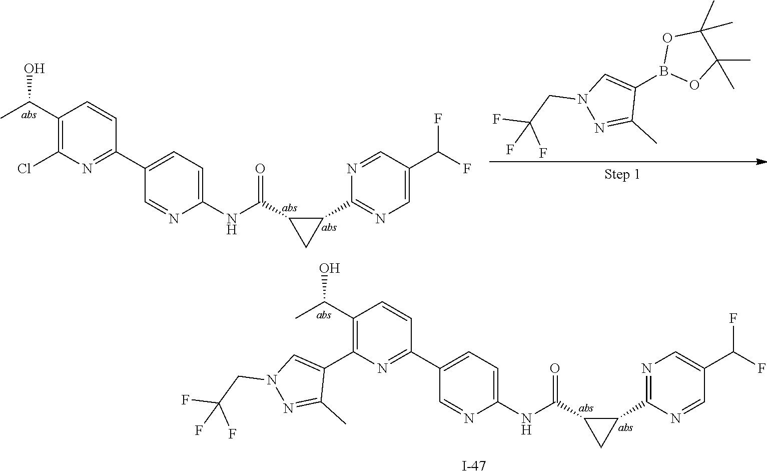

To a solution of (1S,2R)—N-[5-[6-chloro-5-[(1S)-1-hydroxyethyl]-2-pyridyl]-2-pyridyl]-2-[5-(difluoromethyl)pyrimidin-2-yl]cyclopropanecarboxamide (50 mg, 112 mol, 1 eq) and 3-methyl-4-(4,4,5,5-tetramethyl-1,3,2-dioxaborolan-2-yl)-1-(2,2,2-trifluoroethyl)-1H-pyrazole (75 mg, 258 mol, 2.3 eq) in dioxane (20 mL) and H₂O (4 mL) was added Pd(dppf)Cl₂·CH₂Cl₂ (18 mg, 22 mol, 0.2 eq) and K₃PO₄ (72 mg, 336 mol, 3 eq) under N₂ atmosphere. The mixture was stirred at 80° C. for 1 h. The resulting product was filtered and the filtrate was concentrated in vacuum to give a residue. The residue was purified through flash silica gel chromatography (Eluent of EtOAc/PE) then reverse phase preparative HPLC (ACN/H₂O, 0.225% FA) to afford the title compound. ¹H NMR (400 MHz, CDCl₃) δ (ppm): 8.94 (1H), 8.89 (1H), 8.80 (2H), 8.31-8.23 (1H), 8.12 (1H), 8.04 (1H), 7.74 (1H), 7.68 (1H), 6.89-6.56 (1H), 5.07 (1H), 4.82-4.67 (2H), 2.96 (1H), 2.48-2.38 (4H), 2.19 (1H), 2.01-1.79 (1H), 1.76-1.69 (1H), 1.54 (3H). LCMS: 574.3 [M+H]⁺.

### Example 23. Synthesis of (1S,2R)-2-[5-(1-bicyclo[1.1.1]pentanyl)pyrimidin-2-yl]-N-[5-[5-[(1S)-1-hydroxyethyl]-6-[3-methyl-1-(2,2,2-trifluoroethyl)pyrazol-4-yl]-2-pyridyl]-2-pyridyl]cyclopropanecarboxamide (I-152)

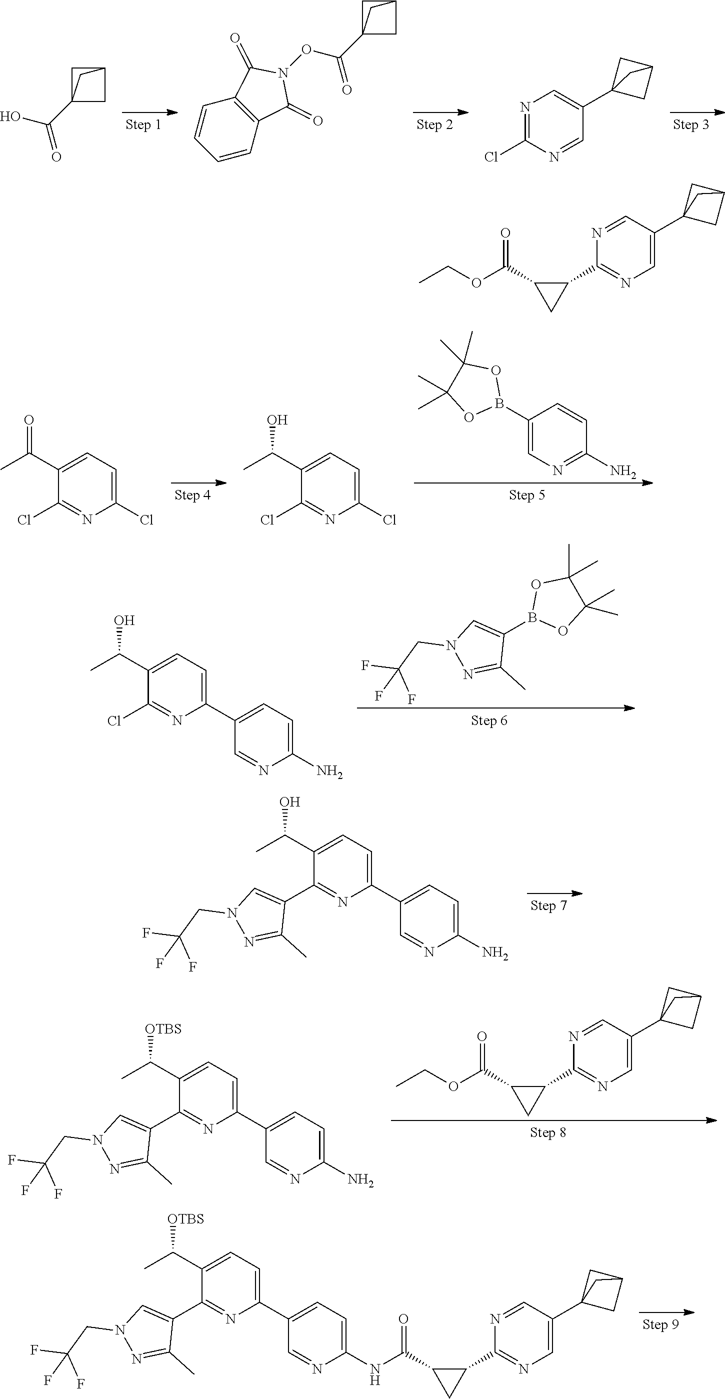

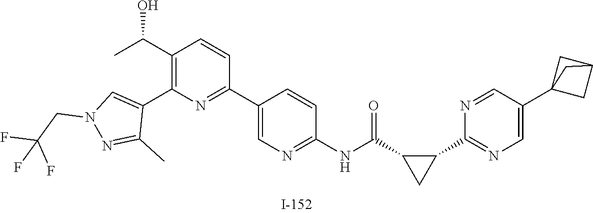 [SMILES: Cc1nn(CC(F)(F)F)cc1-c1nc(-c2ccc(NC(=O)[C@H]3C[C@H]3c3ncc(C45CC(C4)C5)cn3)nc2)ccc1[C@H](C)O]

Step 1. Synthesis of (1,3-dioxoisoindolin-2-yl) bicyclo[1.1.1]pentane-1-carboxylate. To a solution of bicyclo[1.1.1]pentane-1-carboxylic acid (5.00 g, 44.59 mmol, 1.0 eq) and 2-hydroxyisoindoline-1,3-dione (7.27 g, 44.59 mmol, 1.0 eq) in CH₂Cl₂ (100.00 mL) was added DMAP (545 mg, 4.46 mmol, 0.1 eq) and DIC (5.63 g, 44.59 mmol, 1.0 eq) and the mixture was stirred at 15° C. for 16 h. The mixture was concentrated under reduced pressure to give a residue. The residue was purified by flash silica gel chromatography (Eluent of EtOAc/PE) to afford the title compound. ¹H NMR (400 MHz, CDCl3) δ (ppm): 7.95-7.85 (2H), 7.84-7.74 (2H), 2.57 (1H), 2.34 (6H).

Step 2. Synthesis of 5-(1-bicyclo[1.1.1]pentanyl)-2-chloro-pyrimidine. To a solution of (1,3-dioxoisoindolin-2-yl) bicyclo[1.1.1]pentane-1-carboxylate (5.75 g, 22.35 mmol, 1.0 eq) and 5-bromo-2-chloro-pyrimidine (6.49 g, 33.53 mmol, 1.5 eq) in DMA (115 mL) were added NiCl₂(dme) (982.26 mg, 4.47 mmol, 0.2 eq), 5-methoxypyridine-2-carboxamidine hydrochloride (419.39 mg, 2.24 mmol, 0.1 eq) and TBAI (8.26 g, 22.35 mmol, 1.0 eq). Then zinc powder (7.51 g, 114.89 mmol, 5.14 eq) was added to the mixture, then TFA (255 mg, 2.24 mmol, 0.1 eq) was added to the mixture and the mixture was degassed with N₂ 3 times and then stirred at 20° C. for 16 h under N₂. The mixture was filtered and the filtrate was diluted with water (100 mL), extracted with EtOAc (100 mL×3), the organic layer was washed with brine (100 mL), dried over anhydrous Na₂SO₄, filtered and concentrated under reduced pressure to give a residue. The residue was purified by flash silica gel chromatography (Eluent of EtOAc/PE) to afford the title compound. ¹H NMR (400 MHz, CDCl3) δ (ppm): 8.44 (2H), 2.65 (1H), 2.18 (6H). LCMS: 181.2 [M+H]⁺.

Step 3. Synthesis of ethyl (1S,2R)-2-[5-(1-bicyclo[1.1.1]pentanyl)pyrimidin-2-yl]cyclopropanecarboxylate. To a solution of 5-(1-bicyclo[1.1.1]pentanyl)-2-chloro-pyrimidine (1.15 g, 6.37 mmol, 1.0 eq) and ethyl (1S,2R)-2-(4,4,5,5-tetramethyl-1,3,2-dioxaborolan-2-yl)cyclopropanecarboxylate (1.83 g, 7.64 mmol, 1.2 eq) in toluene (15 mL) were added Cs₂CO₃(6.22 g, 19.10 mmol, 3.0 eq), Catacxium A (160 mg, 446 mol, 0.07 eq) and Pd(OAc)₂ (71 mg, 318 mol, 0.05 eq), the mixture was stirred at 100° C. for 2 h under N₂. The mixture was filtered and concentrated under reduced pressure to give a residue which was purified by flash silica gel chromatography (Eluent of EtOAc/PE) to afford the title compound. ¹H NMR (400 MHz, CDCl3) δ (ppm): 8.46 (2H), 4.01-3.92 (2H), 2.85-2.72 (1H), 2.60 (1H), 2.23-2.17 (1H), 2.14 (6H), 2.01-1.95 (1H), 1.49-1.42 (1H), 1.09 (3H). LCMS: 259.2 [M+H]⁺.

Step 4. Synthesis of (S)-1-(2, 6-dichloropyridin-3-yl) ethan-1-ol. To a solution of ketoreductase ES-KRED-262 (30 g, supplier Syncozyme), GDH (10 g), NAD (10 g), NADP (10 g), and D-(+)-Glucose monohydrate (313 g, 1.58 mol, 3 eq) in phosphate buffer (2 L) (prepared by dissolving Na₂HPO₄·12H₂O (104.4 g) and NaH₂PO₄·2H₂O (11.84 g) in 2 L distilled water) was added 1-(2,6-dichloropyridin-3-yl)ethan-1-one (100 g, 526.24 mmol, 1 eq) in MeOH (200 mL). The reaction mixture was stirred at 30° C. for 15 h. The mixture was diluted with EtOAc (2 L) and filtered. The filtrate was adjusted to pH 8 by saturated NaHCO₃ aqueous solution. Then the mixture was extracted with EtOAc (1L×2), washed with brine (500 mL), the organic layer was dried over anhydrous Na₂SO₄, filtered and concentrated under reduced pressure to give a residue. The residue was purified by flash silica gel chromatography (Eluent of PE/EtOAc) to afford the title compound as a single stereoisomer. ¹H NMR (400 MHz, DMSO-d₆) δ (ppm): 8.04 (d, J=8.0 Hz, 1H), 7.60 (d, J=8.0 Hz, 1H), 5.64 (d, J=4.4 Hz, 1H), 5.02-4.80 (m, 1H), 1.32 (d, J=6.4 Hz, 3H). LCMS: 192.2 [M+H]⁺

Step 5. Synthesis of (S)-1-(6′-amino-6-chloro-[2, 3′-bipyridin]-5-yl) ethan-1-ol. To a solution of (1S)-1-(2,6-dichloro-3-pyridyl)ethanol (150 g, 781.08 mmol, 1 eq) in dioxane (1.5 L) and H₂O (300 mL) was added K₃PO₄ (415 g, 1.95 mol, 2.5 eq), Pd(dppf)Cl₂·CH₂Cl₂ (64 g, 78.11 mmol, 0.1 eq) and 5-(4,4,5,5-tetramethyl-1,3,2-dioxaborolan-2-yl)pyridin-2-amine (172 g, 781.08 mmol, 1 eq). The mixture was stirred at 40° C. for 15 h under N₂. The reaction solution was filtered and the filtrate was concentrated under reduced pressure to give a residue. The residue was purified by flash silica gel chromatography (Eluent of PE/EtOAc) to afford the title compound. ¹H NMR (400 MHz, DMSO-d₆) δ=8.62 (d, J=2.0 Hz, 1H), 8.05-7.99 (m, 1H), 7.95 (d, J=8.0 Hz, 1H), 7.85 (d, J=8.0 Hz, 1H), 6.52 (d, J=8.8 Hz, 1H), 6.38 (s, 2H), 5.50 (d, J=4.4 Hz, 1H), 5.01-4.87 (m, 1H), 1.34 (d, J=6.4 Hz, 3H). LCMS: 250.2 [M+H]⁺.

Step 6. Synthesis of (S)-1-(6′-amino-6-(3-methyl-1-(2,2,2-trifluoroethyl)-1H-pyrazol-4-yl)-[2,3′-bipyridin]-5-yl)ethan-1-ol. To a mixture of (1S)-1-[6-(6-amino-3-pyridyl)-2-chloro-3-pyridyl]ethanol (1.45 g, 5.81 mmol, 1 eq), 3-methyl-4-(4,4,5,5-tetramethyl-1,3,2-dioxaborolan-2-yl)-1-(2,2,2-trifluoroethyl)pyrazole (2.02 g, 6.97 mmol, 1.2 eq), K₃PO₄ (3.08 g, 14.52 mmol, 2.5 eq) in dioxane (30 mL) and H₂O (6 mL) was added Pd(dppf)Cl₂·CH₂Cl₂ (474 mg, 581 mol, 0.1 eq). The resulting mixture was degassed and purged with N₂ for 3 times and then was stirred at 80° C. for 1 h under N₂ atmosphere. The reaction mixture was diluted with water (120 mL) at 20° C. and extracted with EtOAc 360 mL (120 mL×3). The combined organic layers were washed with brine (120 mL), dried over anhydrous Na₂SO₄, filtered and concentrated under reduced pressure to give a residue. The residue was purified by flash silica gel chromatography (eluent of DCM/MeOH) to afford the title compound. ¹H NMR (400 MHz, CDCl₃) δ=8.64 (s, 1H), 8.21-8.12 (m, 1H), 7.97 (d, 1H), 7.72 (s, 1H), 7.59 (d, 1H), 6.58 (d, 1H), 5.08-4.97 (m, 1H), 4.76-4.59 (m, 4H), 3.49 (s, 1H), 2.41 (s, 3H), 1.52 (d, 3H). LCMS: 378.2 [M+H]⁺.

Step 7. Synthesis of (S)-5-(1-((tert-butyldimethylsilyl)oxy)ethyl)-6-(3-methyl-1-(2,2,2-trifluoroethyl)-1H-pyrazol-4-yl)-[2,3′-bipyridin]-6′-amine. To a solution of (S)-1-(6′-amino-6-(3-methyl-1-(2,2,2-trifluoroethyl)-1H-pyrazol-4-yl)-[2,3′-bipyridin]-5-yl)ethan-1-ol (1.90 g, 5.03 mmol, 1 eq) in DMF (38 mL) was added imidazole (1.37 g, 20.14 mmol, 4 eq) and TBSCl (2.28 g, 15.10 mmol, 1.86 mL, 3 eq) at 25° C. The mixture was stirred at 25° C. for 1 hr. The reaction mixture was poured into water (100 mL) and extracted with ethyl acetate (100 mL×3). The combined organic layer was washed with brine (100 mL), dried over anhydrous sodium sulfate, filtered and concentrated under reduced pressure to get a residue. The residue was purified by flash silica gel chromatography (eluent of petroleum ether/ethyl acetate) to afford the title compound. ¹H NMR (400 MHz, CDCl₃-d) 6=8.73 (d, 1H), 8.22-8.14 (m, 1H), 8.01 (d, 1H), 7.63 (d, 1H), 7.51 (s, 1H), 6.59 (d, 1H), 5.04-4.93 (m, 1H), 4.78-4.67 (m, 2H), 4.63 (s, 2H), 2.38 (s, 3H), 1.38 (d, 3H), 0.83 (s, 9H), −0.07 (s, 3H), −0.14 (s, 3H). LCMS: 492.1 [M+H]⁺.

Step 8. Synthesis of (1S,2R)-2-[5-(1-bicyclo[1.1.1]pentanyl)pyrimidin-2-yl]-N-[5-[5-[(1S)-1-[tert-butyl(dimethyl)silyl]oxyethyl]-6-[3-methyl-1-(2,2,2-trifluoroethyl)pyrazol-4-yl]-2-pyridyl]-2-pyridyl]cyclopropanecarboxamide. A solution of ethyl (1S,2R)-2-[5-(1-bicyclo[1.1.1]pentanyl)pyrimidin-2-yl]cyclopropanecarboxylate (79 mg, 305 mol, 1.5 eq) and 5-[5-[(1S)-1-[tert-butyl(dimethyl)silyl]oxyethyl]-6-[3-methyl-1-(2,2,2-trifluoroethyl)pyrazol-4-yl]-2-pyridyl]pyridin-2-amine (100 mg, 203 mol, 1.0 eq) in THF (5 mL) was added AlMe₃ (2 M in toluene, 610 μL, 6.0 eq) at 0° C. under N₂ atmosphere, then the mixture was stirred at 80° C. for 16 h. The reaction mixture was quenched by addition of saturated NH₄Cl solution (20 mL) at 20° C. and then diluted with water (20 mL) and extracted with EtOAc (15 mL×3). The combined organic layers were washed with brine (8 mL×2), dried over anhydrous Na₂SO₄, filtered and concentrated under reduced pressure to give a residue. The residue was purified by flash silica gel chromatography (EtOAc/PE, then MeOH/DCM) to afford the title compound. LCMS: 704.5 [M+H]⁺.

Step 9. Synthesis of (1S,2R)-2-[5-(1-bicyclo[1.1.1]pentanyl)pyrimidin-2-yl]-N-[5-[5-[(1S)-1-hydroxyethyl]-6-[3-methyl-1-(2,2,2-trifluoroethyl)pyrazol-4-yl]-2-pyridyl]-2-pyridyl]cyclopropanecarboxamide. To a solution of (1S,2R)-2-[5-(1-bicyclo[1.1.1]pentanyl)pyrimidin-2-yl]-N-[5-[5-[(1S)-1-[tert-butyl(dimethyl)silyl]oxyethyl]-6-[3-methyl-1-(2,2,2-trifluoroethyl)pyrazol-4-yl]-2-pyridyl]-2-pyridyl]cyclopropanecarboxamide (60 mg, 85 mol, 1.0 eq) in THF (2 mL) was added TBAF (85 μl, 1M in THF, 1.0 eq), the mixture was stirred at 25° C. for 16 h. The mixture was concentrated under reduced pressure to give a residue. The residue was purified by flash silica gel chromatography (Eluent of EtOAc/PE, then MeOH/DCM), and then further purified by preparative HPLC (ACN/H₂O, 0.05% NH₄HCO₃) to afford the title compound. ¹H NMR (400 MHz, CDCl3) δ (ppm): 9.35 (1H), 8.93 (1H), 8.49 (2H), 8.27-8.21 (1H), 8.17-8.13 (1H), 8.03 (1H), 7.73 (1H), 7.66 (1H), 5.09-5.00 (1H), 4.77-4.65 (2H), 2.91-2.79 (1H), 2.60 (1H), 2.42 (3H), 2.37-2.28 (1H), 2.13 (6H), 2.11-2.07 (1H), 1.96-1.84 (1H), 1.65-1.62 (1H), 1.53 (3H). LCMS: 590.3 [M+H]⁺.

### Example 24. Synthesis of (1S,2R)—N-(6-(3-(difluoromethyl)-1-(2,2,2-trifluoroethyl)-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide (I-153)

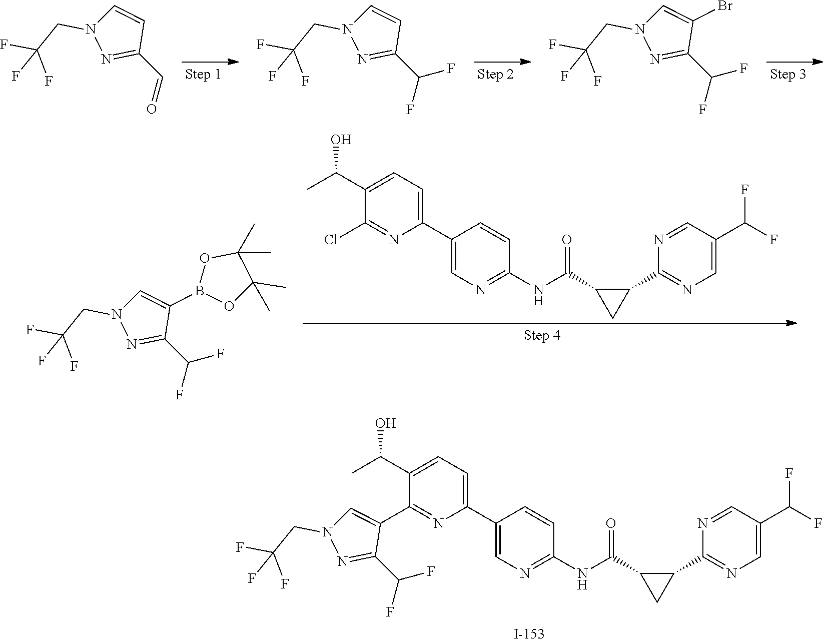 [SMILES: O=Cc1ccn(CC(F)(F)F)n1 ; FC(F)c1ccn(CC(F)(F)F)n1 ; FC(F)c1nn(CC(F)(F)F)cc1Br ; CC1(C)OB(c2cn(CC(F)(F)F)nc2C(F)F)OC1(C)C ; C[C@H](O)c1ccc(-c2ccc(NC(=O)[C@H]3C[C@H]3c3ncc(C(F)F)cn3)nc2)nc1Cl ; C[C@H](O)c1ccc(-c2ccc(NC(=O)[C@H]3C[C@H]3c3ncc(C(F)F)cn3)nc2)nc1-c1cn(CC(F)(F)F)nc1C(F)F]

Step 1. Synthesis of 3-(difluoromethyl)-1-(2,2,2-trifluoroethyl)-1H-pyrazole. To a solution of 1-(2,2,2-trifluoroethyl)pyrazole-3-carbaldehyde (850 mg, 4.77 mmol, 1 eq) in CH₂Cl₂ (20 mL) was added DAST (1.92 g, 11.93 mmol, 1.58 mL, 2.5 eq) at 0° C. under N₂ atmosphere. The mixture was stirred at 30° C. for 2 h. The mixture was quenched with water (20 ml) and then extracted with CH₂Cl₂ (15 ml×3). The combined organic layers were washed with water (10 ml). The organic layer was separated, dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 7.56 (1H), 6.88-6.51 (2H), 4.78-4.65 (2H). LCMS: 201.2 [M+H]⁺.

Step 2. Synthesis of 4-bromo-3-(difluoromethyl)-1-(2,2,2-trifluoroethyl)-1H-pyrazole. To a solution of 3-(difluoromethyl)-1-(2,2,2-trifluoroethyl)pyrazole (700 mg, 3.50 mmol, 1 eq) in DMF (20 mL) was added NBS (1.87 g, 10.49 mmol, 3 eq). The mixture was stirred at 60° C. for 2 h. The mixture was diluted with water (50 ml) and then extracted with EtOAc (15 ml×3). The combined organic layers were washed with water (15 ml). The organic layers were separated, dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 7.61 (1H), 6.88-6.51 (1H), 4.78-4.64 (2H). LCMS: 278.9 [M+H]⁺.

Step 3. Synthesis of 3-(difluoromethyl)-4-(4,4,5,5-tetramethyl-1,3,2-dioxaborolan-2-yl)-1-(2,2,2-trifluoroethyl)-1H-pyrazole. To a solution of 4-bromo-3-(difluoromethyl)-1-(2,2,2-trifluoroethyl)pyrazole (700 mg, 2.51 mmol, 1 eq) and 4,4,4′,4′,5,5,5′,5′-octamethyl-2,2′-bi(1,3,2-dioxaborolane) (956 mg, 3.76 mmol, 1.5 eq) in toluene (10 mL) was added Cy3P—Pd-G3 (184 mg, 251 mol, 0.1 eq) and KOAc (492 mg, 5 mmol, 2 eq) at 25° C. under N₂ atmosphere. The mixture was stirred at 90° C. for 16 h. The mixture was cooled to room temperature, diluted with water (10 ml) and then extracted with EtOAc (5 ml×3). The combined organic layers were washed with water (5 ml). The organic layer was dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 7.82 (1H), 7.11-6.77 (1H), 4.81-4.66 (2H), 1.33 (12H). LCMS: 327.2 [M+H]⁺.

Step 4. Synthesis of (1S,2R)—N-(6-(3-(difluoromethyl)-1-(2,2,2-trifluoroethyl)-1H-pyrazol-4-yl)-5-((S)-1-hydroxyethyl)-[2,3′-bipyridin]-6′-yl)-2-(5-(difluoromethyl)pyrimidin-2-yl)cyclopropane-1-carboxamide. To a solution of (1S,2R)—N-[5-[6-chloro-5-[(1S)-1-hydroxyethyl]-2-pyridyl]-2-pyridyl]-2-[5-(difluoromethyl)pyrimidin-2-yl]cyclopropanecarboxamide (80 mg, 180 mol, 1 eq) and 3-(difluoromethyl)-4-(4,4,5,5-tetramethyl-1,3,2-dioxaborolan-2-yl)-1-(2,2,2-trifluoroethyl)pyrazole (117 mg, 359 mol, 2 eq) in dioxane (1 mL) and H₂O (0.2 mL) was added K₃PO₄ (114 mg, 538 mol, 3 eq) and Pd(dppf)Cl₂·CH₂Cl₂ (15 mg, 18 mol, 0.1 eq) at 25° C. under N₂ atmosphere. The mixture was stirred at 80° C. for 3 h. The mixture was cooled to room temperature and then concentrated under reduced pressure to give a residue. The residue was purified by flash silica gel chromatography (Eluent of DCM/MeOH) and reverse phase preparative HPLC (ACN/H₂O, 0.05% NH₄HCO₃) to afford the title compound. ¹H NMR (400 MHz, CD₃OD) δ (ppm): 8.94 (1H), 8.82 (2H), 8.38-8.30 (1H), 8.15 (1H), 8.13 (1H), 7.94-7.86 (2H), 7.18-6.76 (2H), 5.15-5.06 (2H), 5.02-4.96 (1H), 3.00-2.89 (1H), 2.63-2.51 (1H), 2.13-2.05 (1H), 1.65-1.55 (1H), 1.44 (3H). LCMS: 610.2 [M+H]⁺.

### Example 25. Synthesis of N-[5-[5-[(1S)-1-hydroxyethyl]-6-[3-methyl-1-(2,2,2-trifluoroethyl) pyrazol-4-yl]-2-pyridyl]-2-pyridyl]-3-(5-methyl-2-pyridyl) propanamide (I-154)

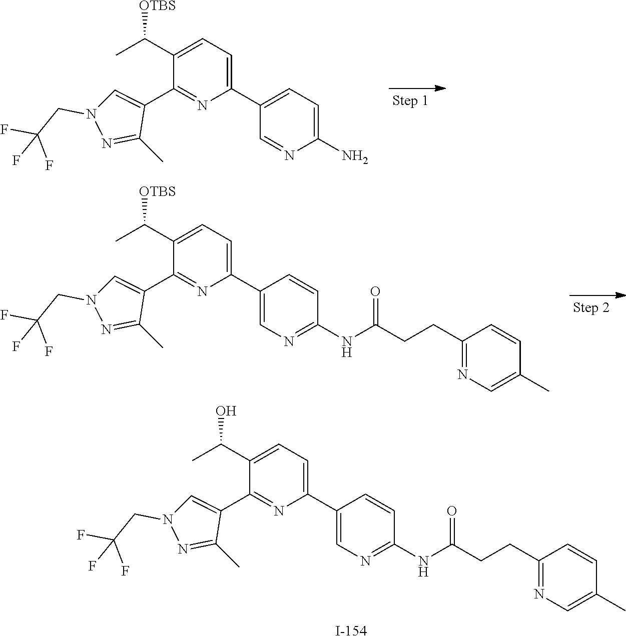

Step 1. Synthesis of N-[5-[5-[(1S)-1-[tert-butyl(dimethyl) silyl]oxyethyl]-6-[3-methyl-1-(2,2,2-trifluoroethyl) pyrazol-4-yl]-2-pyridyl]-2-pyridyl]-3-(5-methyl-2-pyridyl) propenamide. To a solution of 5-[5-[(1S)-1-[tert-butyl(dimethyl)silyl]oxyethyl]-6-[3-methyl-1-(2,2,2-trifluoroethyl) pyrazol-4-yl]-2-pyridyl]pyridin-2-amine (50 mg, 102 mol, 1 eq) in pyridine (1 mL) was added HATU (58 mg, 152 mol, 1.5 eq) and 3-(5-methyl-2-pyridyl) propanoic acid (25 mg, 152 mol, 1.5 eq). The mixture was stirred at 80° C. for 4 h. After cooling down to room temperature, H₂O (30 mL) was added into the reaction mixture and it was extracted with EtOAc (40 mL×2). The combined organic layers were washed with brine (20 mL), dried over anhydrous Na₂SO₄, filtered and concentrated under reduced pressure to give a residue. The residue was purified by flash silica gel chromatography (Eluent of PE/EtOAc) to afford the title compound. LCMS: 639.4 [M+H]⁺.

Step 2. Synthesis of N-[5-[5-[(1S)-1-hydroxyethyl]-6-[3-methyl-1-(2,2,2-trifluoroethyl) pyrazol-4-yl]-2-pyridyl]-2-pyridyl]-3-(5-methyl-2-pyridyl) propenamide. To a solution of N-[5-[5-[(1S)-1-[tert-butyl(dimethyl)silyl]oxyethyl]-6-[3-methyl-1-(2,2,2-trifluoroethyl) pyrazol-4-yl]-2-pyridyl]-2-pyridyl]-3-(5-methyl-2-pyridyl) propanamide (60 mg, 94 mol, 1 eq) in THF (1 mL) was added TBAF (1 M in THF, 188 μL, 2 eq). The mixture was stirred at 25° C. for 1 h. The reaction solution was filtered and the filtrate was concentrated under reduced pressure to give the crude product. The residue was purified by flash silica gel chromatography (Eluent of DCM/MeOH) and reverse phase preparative HPLC (ACN/H₂O, 0.05% NH₄OH) to afford the title compound. ¹H NMR (400 MHz, CD₃OD) δ (ppm): 8.97-8.90 (1H), 8.40-8.36 (1H), 8.30 (1H), 8.20 (1H), 8.13 (1H), 7.94 (1H), 7.87 (1H), 7.61-7.56 (1H), 7.27 (1H), 5.01-4.91 (3H), 3.17-3.11 (2H), 2.90-2.84 (2H), 2.34 (3H), 2.32 (3H), 1.42 (3H). LCMS: 525.3 [M+H]⁺.

### Example 26. Synthesis of (1S,2R)-2-(5-((1S,4s)-2-oxabicyclo[2.1.1]hexan-4-yl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (I-155)

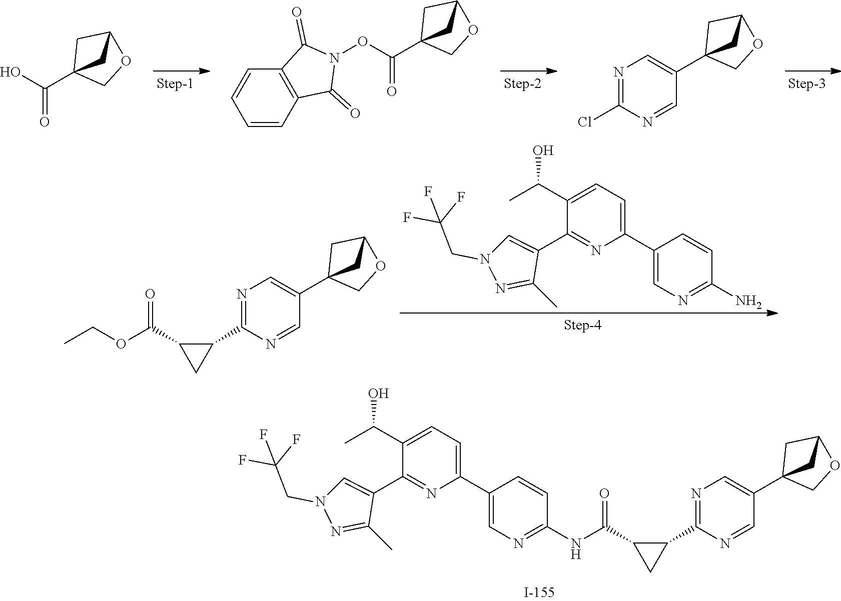 [SMILES: O=C(O)[C@]12CO[C@H](C1)C2 ; O=C1c2ccccc2C(=O)N1OC(=O)[C@]12CO[C@H](C1)C2 ; Clc1ncc([C@]23CO[C@H](C2)C3)cn1 ; CCOC(=O)[C@H]1C[C@H]1c1ncc([C@]23CO[C@H](C2)C3)cn1 ; Cc1nn(CC(F)(F)F)cc1-c1nc(-c2ccc(N)nc2)ccc1[C@H](C)O ; Cc1nn(CC(F)(F)F)cc1-c1nc(-c2ccc(NC(=O)[C@H]3C[C@H]3c3ncc([C@]45CO[C@H](C4)C5)cn3)nc2)ccc1[C@H](C)O]

Step 1. Synthesis of (1, 3-dioxoisoindolin-2-yl) 2-oxabicyclo[2.1.1]hexane-4-carboxylate. To a solution of 2-oxabicyclo[2.1.1]hexane-4-carboxylic acid (1.5 g, 11.71 mmol, 1 eq), DMAP (29 mg, 234 mol, 0.02 eq) and 2-hydroxyisoindoline-1, 3-dione (1.91 g, 11.71 mmol, 1 eq) in DCM (20 mL) was added dropwise a solution of DCC (2.66 g, 12.88 mmol, 2.60 mL, 1.1 eq) in DCM (20 mL). The mixture was stirred at 30° C. under N₂ atmosphere for 15 h. The mixture was filtered and concentrated under reduced pressure. The residue was purified by column chromatography (Eluent of PE/EtOAc) to afford the title compound. LCMS: 274.1 [M+H]⁺.

Step 2. Synthesis of 2-chloro-5-(2-oxabicyclo[2.1.1]hexan-4-yl) pyrimidine. To a solution of 5-bromo-2-chloro-pyrimidine (3.19 g, 16.47 mmol, 1.5 eq), (1, 3-dioxoisoindolin-2-yl) 2-oxabicyclo[2.1.1]hexane-4-carboxylate (3.00 g, 10.98 mmol, 1 eq), 5-methoxypyridine-2-carboxamidine hydrochloride (206 mg, 1.10 mmol, 0.1 eq), NiCl₂(dme) (482 mg, 2.20 mmol, 0.2 eq) and TBAI (4.06 g, 10.98 mmol, 1 eq) in DMA (30 mL) was added Zn (3.96 g, 60.56 mmol, 5.52 eq) and TFA (125 mg, 1.10 mmol, 82 μL, 0.1 eq) under N₂ atmosphere. The mixture was stirred at 20° C. under N₂ atmosphere for 15 h. The mixture was filtered and the filtrate was diluted with water (200 mL) and extracted with EtOAc (100 mL×3). The combined organic layers were washed with brine (200 mL), dried over anhydrous Na₂SO₄, filtered and concentrated under reduced pressure to give a residue. The residue was purified by column chromatography (Eluent of PE/EtOAc) to obtained the title compound. LCMS: 197.1 [M+H]⁺.

Step 3. Synthesis of Ethyl (1S, 2R) −2-[5-(2-oxabicyclo[2.1.1]hexan-4-yl) pyrimidin-2-yl]cyclopropanecarboxylate. To a solution of 2-chloro-5-(2-oxabicyclo[2.1.1]hexan-4-yl) pyrimidine (0.7 g, 3.56 mmol, 1 eq) and ethyl (1S, 2R) −2-(4, 4, 5, 5-tetramethyl-1, 3, 2-dioxaborolan-2-yl) cyclopropanecarboxylate (855 mg, 3.56 mmol, 1 eq) in dioxane (10 mL) and H₂O (2 mL) was added Pd(OAc)₂ (160 mg, 712 mol, 0.2 eq), Catacxium A (383 mg, 1.07 mmol, 0.3 eq) and Cs₂CO₃ (3.48 g, 10.68 mmol, 3 eq) under N₂ atmosphere. The mixture was stirred at 100° C. under N₂ atmosphere for 15 h. The mixture was filtered and concentrated under reduced pressure to give a residue. The residue was purified by column chromatography (Eluent of PE/EtOAc) to afford the title compound. ¹H NMR (400 MHz, CDCl₃) δ (ppm): 8.56 (2H), 4.72 (1H), 3.96-4.06 (2H), 3.91 (2H), 2.83-2.80 (1H), 2.20-2.32 (1H), 1.99-2.13 (5H), 1.51-1.48 (1H), 1.13 (3H). LCMS: 275.1 [M+H]⁺.

Step 4. Synthesis of (1S,2R)-2-(5-((1S,4s)-2-oxabicyclo[2.1.1]hexan-4-yl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a solution of (1S)-1-[6-(6-amino-3-pyridyl)-2-[3-methyl-1-(2,2,2-trifluoroethyl)pyrazol-4-yl]-3-pyridyl]ethanol (80 mg, 212.00 mol, 1 eq) in THF (0.7 mL) was added AlMe₃ (2 M in toluene, 530 μL, 5 eq) at 0° C. under N₂ atmosphere. The mixture was stirred at 40° C. for 1 h. Ethyl (1S,2R)-2-[5-(2-oxabicyclo[2.1.1]hexan-4-yl)pyrimidin-2-yl]cyclopropanecarboxylate (58 mg, 212 mol, 1 eq) in THF (0.7 mL) was added to the mixture at 20° C. under N₂ atmosphere. The resulting mixture was stirred at 90° C. for another 14 h. The mixture was cooled to room temperature, quenched with saturated NaHCO₃ aqueous solution (15 mL) under N₂ atmosphere. The resulting mixture was filtered. The filter cake was washed with DCM:MeOH=5:1 (2×5 mL). The combined filtrate was extracted with DCM:MeOH=5:1 (3×10 mL). The combined organic layer was washed with brine (10 mL), dried over anhydrous Na₂SO₄, filtered and concentrated under reduced pressure to give a residue. The residue was purified by reverse phase preparative HPLC (ACN/H₂O, 0.05% NH₄HCO₃) to afford the title compound. ¹H NMR (400 MHz, CD₃OD) δ (ppm): 8.91 (1H), 8.62 (2H), 8.31-8.24 (1H), 8.11 (1H), 7.93 (1H), 7.91 (1H), 7.83 (1H), 4.99-4.96 (1H), 4.95 (2H), 4.65-4.63 (1H), 3.88 (2H), 2.92-2.82 (1H), 2.58-2.47 (1H), 2.32 (3H), 2.17 (2H), 2.08-2.00 (1H), 1.92 (1H), 1.91 (1H), 1.59-1.52 (1H), 1.41 (3H).

LCMS: 606.2 [M+H]⁺.

### Example 27. Synthesis of (1S,2R)-2-(5-(1-(dimethylamino)cyclopropyl)pyridin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (I-156)

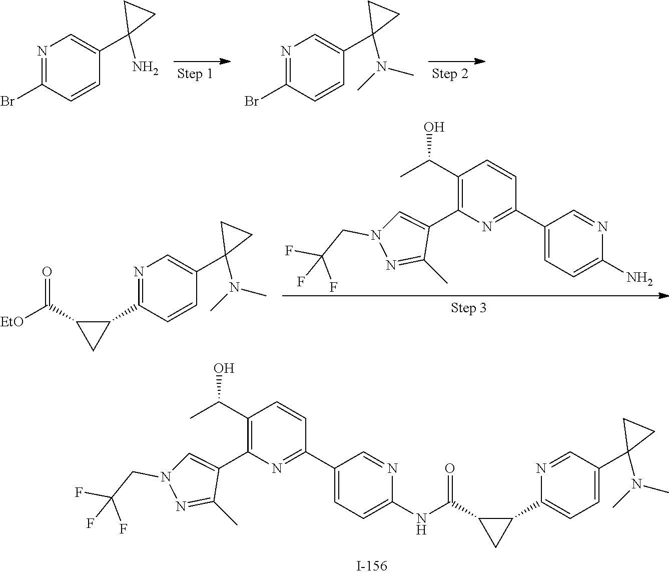

Step 1. Synthesis of 1-(6-bromopyridin-3-yl)-N, N-dimethylcyclopropan-1-amine. To a solution of 1-(6-bromo-3-pyridyl) cyclopropanamine (950 mg, 4.46 mmol, 1 eq) and HCHO (22.29 mmol, 1.66 mL, 37% aqeuous, 5 eq) in MeOH (10 mL) was added AcOH (268 mg, 4.46 mmol, 1 eq) and the resulting mixture was stirred at 25° C. for 0.5 h. Then NaBH₃CN (841 mg, 13.38 mmol, 3 eq) was added and the reaction mixture was stirred at 25° C. for 1 h. The mixture was poured into saturated NaHCO₃ aqueous solution (40 mL) and extracted with EtOAc (20 mL×3). The combined organic layers were washed with brine (10 mL), dried over anhydrous Na₂SO₄, filtered and concentrated under reduced pressure to give a residue. The residue was purified by silica gel column chromatography (eluent of PE/EtOAc) and preparative-HPLC (ACN/H₂O, 0.05% NH₄OH) to afford the title compound. ¹H NMR (400 MHz, CDCl₃) δ (ppm): 8.30 (1H), 7.47 (2H), 2.23 (6H), 1.06-0.98 (2H), 0.85-0.76 (2H).

Step 2. Synthesis of ethyl (1S,2R)-2-(5-(1-(dimethylamino)cyclopropyl)pyridin-2-yl)cyclopropane-1-carboxylate. To a solution of 1-(6-bromo-3-pyridyl)-N, N-dimethyl-cyclopropanamine (300 mg, 1.24 mmol, 1 eq) and ethyl (1S, 2R) −2-(4, 4, 5, 5-tetramethyl-1, 3, 2-dioxaborolan-2-yl) cyclopropanecarboxylate (329 mg, 1.37 mmol, 1.1 eq) in dioxane (6 mL) and H₂O (1.5 mL) was added Pd(OAc)₂ (112 mg, 498 mol, 0.4 eq), Cs₂CO₃ (1.22 g, 3.73 mmol, 3 eq), cataCXium A (268 mg, 746 mol, 0.6 eq), then the solution was stirred at 100° C. for 16 h under N₂ atmosphere. The mixture was poured into water (20 mL). The mixture was extracted with EtOAc (10 mL×2). The combined organic layers were washed with brine (10 mL), dried over anhydrous Na₂SO₄, filtered and concentrated under reduced pressure to give a residue. The residue was purified by preparative-HPLC (ACN/H₂O, 0.05% NH₄OH) to afford the title compound. LCMS: 275.2 [M+H]⁺.

Step 3. Synthesis of (1S,2R)-2-(5-(1-(dimethylamino)cyclopropyl)pyridin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a solution of AlMe₃ (2 M in toluene, 994 μL, 1.99 mmol, 5 eq) was added dropwise a solution of (S)-1-(6′-amino-6-(3-methyl-1-(2,2,2-trifluoroethyl)-1H-pyrazol-4-yl)-[2,3′-bipyridin]-5-yl)ethan-1-ol (150 mg, 398 mol, 1 eq) and ethyl (1S,2R)-2-[5-[1-(dimethylamino)cyclopropyl]-2-pyridyl]cyclopropanecarboxylate (109 mg, 398 mol, 1 eq) in dioxane (1.5 mL) at 90° C. under N₂ atmosphere. The resulting mixture was stirred at 90° C. for 1 h. The mixture was cooled to 0° C., quenched with saturated NH₄Cl aqueous solution (2 mL) under N₂ atmosphere and extracted with EtOAc (3×5 mL). The organic layer was washed with brine (10 ml), dried over anhydrous Na₂SO₄, filtered and concentrated under reduced pressure to give a residue. The residue was purified by preparative-HPLC (ACN/H₂O, 0.05% NH₄OH) to afford the title compound. ¹H NMR (400 MHz, CD₃OD) δ (ppm): 8.90 (1H), 8.35 (1H), 8.28-8.22 (1H), 8.11 (1H), 7.93 (1H), 7.87 (1H), 7.82 (1H), 7.72-7.65 (1H), 7.40 (1H), 4.99-4.90 (3H), 2.83-2.70 (1H), 2.53-2.45 (1H), 2.32 (3H), 2.15 (6H), 1.95-1.89 (1H), 1.56-1.48 (1H), 1.41 (3H), 0.98-0.92 (2H), 0.86-0.79 (2H). LCMS: 606.5 [M+H]⁺.

### Example 28. Synthesis of (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 2) (I-157 & I-158)

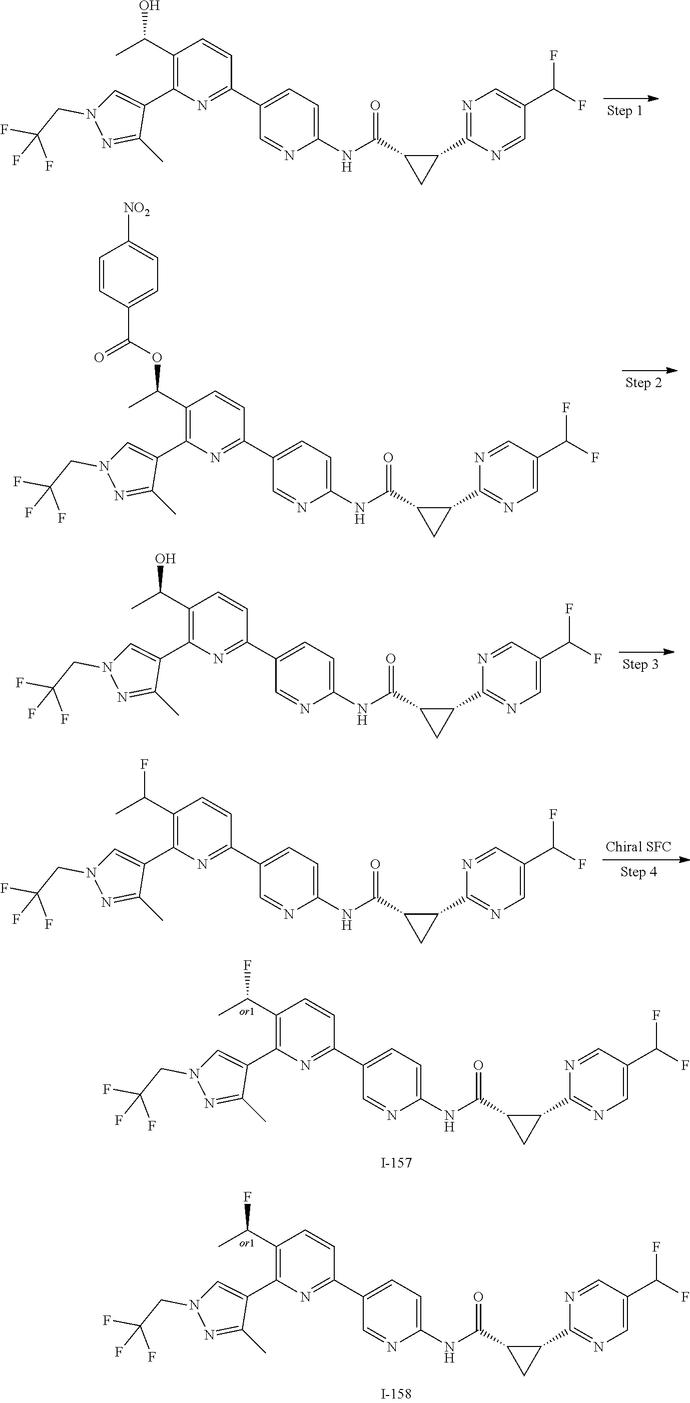

Step 1. Synthesis of (R)-1-(6′-((1S,2R)-2-(5-(difluoromethyl)pyrimidin-2-yl)cyclopropane-1-carboxamido)-6-(3-methyl-1-(2,2,2-trifluoroethyl)-1H-pyrazol-4-yl)-[2,3′-bipyridin]-5-yl)ethyl 4-nitrobenzoate. To a solution of (1S,2R)-2-[5-(difluoromethyl)pyrimidin-2-yl]-N-[5-[5-[(1S)-1-hydroxyethyl]-6-[3-methyl-1-(2,2,2-trifluoroethyl)pyrazol-4-yl]-2-pyridyl]-2-pyridyl]cyclopropanecarboxamide (200 mg, 348 μmol, 1 eq) and 4-nitrobenzoic acid (87 mg, 523 μmol, 1.5 eq) in THF (2 mL) was added PPh₃ (201 mg, 767 μmol, 2.2 eq) and DIAD (148 mg, 732 μmol, 141 μL, 2.1 eq) under N₂ atmosphere at 0° C. The mixture was stirred at 25° C. for 1 h. The mixture was diluted with H₂O (10 mL) and extracted with EtOAc (3×20 mL). The combined organic layer was washed with brine (10 ml), dried over anhydrous Na₂SO₄, filtered and concentrated under reduced pressure. The residue was purified by flash silica gel chromatography (Eluent of DCM/MeOH) to afford the title compound. LCMS: 723.3 [M+H]⁺.

Step 2. Synthesis of (1S,2R)-2-(5-(difluoromethyl)pyrimidin-2-yl)-N-(5-((R)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a solution of (R)-1-(6′-((1S,2R)-2-(5-(difluoromethyl)pyrimidin-2-yl)cyclopropane-1-carboxamido)-6-(3-methyl-1-(2,2,2-trifluoroethyl)-1H-pyrazol-4-yl)-[2,3′-bipyridin]-5-yl)ethyl 4-nitrobenzoate (380 mg, 279 μmol, 1 eq) in THF (4 mL) and H₂O (0.8 mL) was added LiOH·H₂O (14 mg, 334 μmol, 1.2 eq). The mixture was stirred at 0° C. for 1 h. The mixture was diluted with H₂O (10 mL) and extracted with EtOAc (3×20 mL). The combined organic layer was washed with brine (10 ml), dried over anhydrous Na₂SO₄, filtered and concentrated under reduced pressure. The residue was purified by flash silica gel chromatography (Eluent of 0-20% DCM/MeOH) to afford the title compound. LCMS: 574.3 [M+H]⁺

Step 3. Synthesis of (1S,2R)-2-(5-(difluoromethyl)pyrimidin-2-yl)-N-(5-(1-fluoroethyl)-6-(3-methyl-1-(2,2,2-trifluoroethyl)-1H-pyrazol-4-yl)-[2,3′-bipyridin]-6′-yl)cyclopropane-1-carboxamide. To a solution of (1S,2R)-2-[5-(difluoromethyl)pyrimidin-2-yl]-N-[5-[5-[(1R)-1-hydroxyethyl]-6-[3-methyl-1-(2,2,2-trifluoroethyl)pyrazol-4-yl]-2-pyridyl]-2-pyridyl]cyclopropanecarboxamide (50 mg, 87 mol, 1 eq) in DCM (0.5 mL) was added DAST (31 mg, 192 mol, 25 μL, 2.2 eq) under N₂ atmosphere at −20° C. The mixture was stirred at −20° C. for 0.5 h. The mixture was quenched with H₂O (10 mL) and extracted with DCM (3×2 mL), the organic layers was washed with brine (5 ml), dried over anhydrous Na₂SO₄, filtered and concentrated under reduced pressure. The residue was purified by preparative HPLC (ACN/H₂O, 0.225% FA) to afford the title compound. ¹H NMR (400 MHz, CD₃OD) δ (ppm): 8.95 (1H), 8.82 (2H), 8.35-8.30 (1H), 8.08 (1H), 7.97-7.91 (2H), 7.87 (1H), 6.92 (1H), 5.84-5.63 (1H), 5.00-4.91 (2H), 2.94 (1H), 2.63-2.53 (1H), 2.35 (3H), 2.14-2.04 (1H), 1.69-1.56 (4H). LCMS: 576.3 [M+H]⁺.

Step 4. Synthesis of (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 2). (1S,2R)-2-(5-(difluoromethyl)pyrimidin-2-yl)-N-(5-(1-fluoroethyl)-6-(3-methyl-1-(2,2,2-trifluoroethyl)-1H-pyrazol-4-yl)-[2,3′-bipyridin]-6′-yl)cyclopropane-1-carboxamide was separated by chiral SFC (SFC Preparative Method: Instrument: Waters 150 Preparative SFC system; Column: DAICEL Chiralpak Phenomenex-Cellulose-2 250×30 mm I.D., 10 m particle size; Mobilephase A: CO₂, Mobile Phase B: IPA:ACN (0.1% NH₄OH); Isocratic elution: 25% Phase B in Supercritical CO₂; Flow rate: 150 g/min; Retention time: Peak1: 2.77 min, Peak2: 3.82 min; Back Pressure: 80 bar to keep the CO₂ in Supercritical flow; Wave Length: 220 nm) to give the title compounds as peak 1 (Isomer 1) and peak 2 (Isomer 2).

Peak 1: (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 1) ¹H NMR (400 MHz, CD₃OD) δ (ppm): 8.95 (1H), 8.82 (2H), 8.37-8.29 (1H), 8.08 (1H), 7.93 (2H), 7.87 (1H), 6.92 (1H), 5.84-5.63 (1H), 5.01-4.91 (2H), 3.02-2.89 (1H), 2.65-2.52 (1H), 2.35 (3H), 2.15-2.03 (1H), 1.67-1.59 (3H), 1.59 (1H). LCMS: 576.3 [M+H]⁺. Chiral SFC: Rt=0.836 min (instrument: SHIMADZU LC-30ADsf; column: Cellulose-2 Lux@3 μm Cellulose-2, 50*4.6 mm; mobile phase A: CO₂; mobile phase B: iPrOH & MeCN (0.05% DEA); flow rate: 3 mL/min; 35° C.; 100 bar; 220 nm).

Peak 2: (1S,2R)-2-(5-(difluoromethyl)pyrimidin-2-yl)-N-(5-((R*)-1-fluoroethyl)-6-(3-methyl-1-(2,2,2-trifluoroethyl)-1H-pyrazol-4-yl)-[2,3′-bipyridin]-6′-yl)cyclopropane-1-carboxamide (Isomer 2) ¹H NMR (400 MHz, CD₃OD) δ (ppm): 8.95 (1H), 8.82 (2H), 8.37-8.28 (1H), 8.08 (1H), 7.97-7.90 (2H), 7.87 (1H), 7.08-6.76 (1H), 5.85-5.64 (1H), 5.00-4.91 (2H), 2.99-2.89 (1H), 2.58 (1H), 2.35 (3H), 2.14-2.05 (1H), 1.67-1.59 (3H), 1.57 (1H). LCMS: 576.3 [M+H]⁺. Chiral SFC: Rt=0.997 min (instrument: SHIMADZU LC-30ADsf; column: Cellulose-2 Lux@3 μm Cellulose-2, 50*4.6 mm; mobile phase A: CO₂; mobile phase B: iPrOH & MeCN (0.05% DEA); flow rate: 3 mL/min; 35° C.; 100 bar; 220 nm).

### Example 29. Synthesis of (1S,2R)-2-(5-(difluoromethyl)pyrimidin-2-yl)-N-(5-((S)-1-hydroxyethyl)-6-(5-methyl-1-(2,2,2-trifluoroethyl)-1H-1,2,3-triazol-4-yl)-[2,3′-bipyridin]-6′-yl)cyclopropane-1-carboxamide & (1S,2R)-2-(5-(difluoromethyl)pyrimidin-2-yl)-N-(5-((S)-1-hydroxyethyl)-6-(5-methyl-2-(2,2,2-trifluoroethyl)-2H-1,2,3-triazol-4-yl)-[2,3′-bipyridin]-6′-yl)cyclopropane-1-carboxamide (I-159 & I-160)

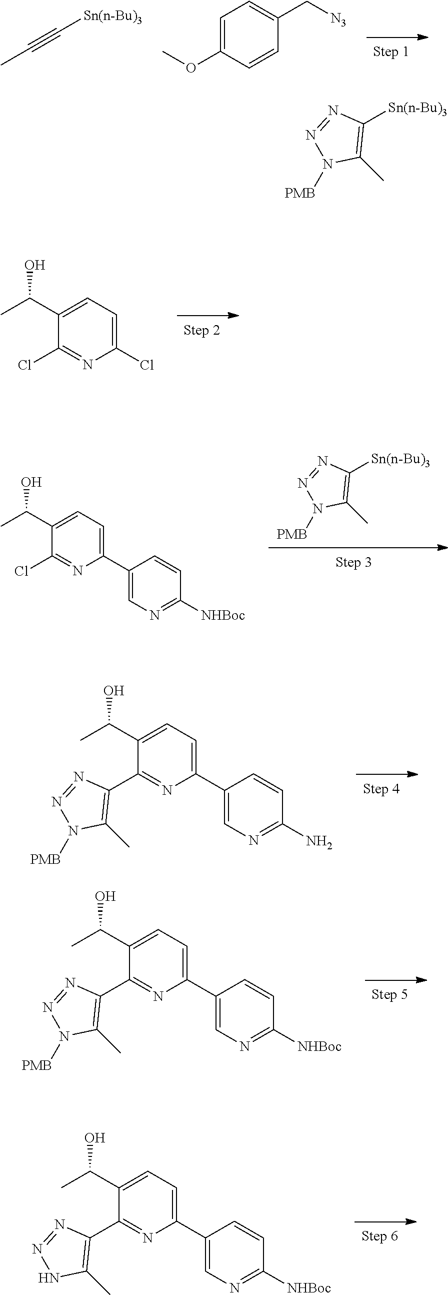

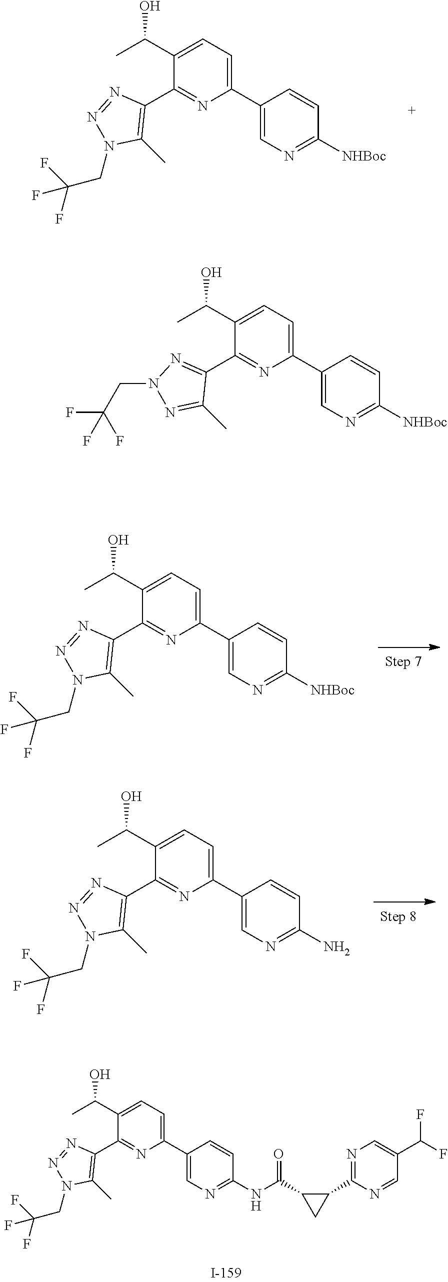 [SMILES: Cc1c(-c2nc(-c3ccc(NC(=O)OC(C)(C)C)nc3)ccc2[C@H](C)O)nnn1CC(F)(F)F ; Cc1nn(CC(F)(F)F)nc1-c1nc(-c2ccc(NC(=O)OC(C)(C)C)nc2)ccc1[C@H](C)O ; Cc1c(-c2nc(-c3ccc(NC(=O)OC(C)(C)C)nc3)ccc2[C@H](C)O)nnn1CC(F)(F)F ; Cc1c(-c2nc(-c3ccc(N)nc3)ccc2[C@H](C)O)nnn1CC(F)(F)F ; Cc1c(-c2nc(-c3ccc(NC(=O)[C@H]4C[C@H]4c4ncc(C(F)F)cn4)nc3)ccc2[C@H](C)O)nnn1CC(F)(F)F]

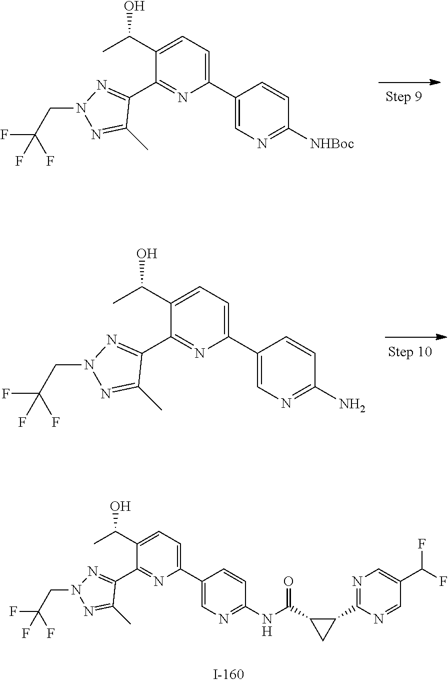 [SMILES: Cc1nn(CC(F)(F)F)nc1-c1nc(-c2ccc(NC(=O)OC(C)(C)C)nc2)ccc1[C@H](C)O ; Cc1nn(CC(F)(F)F)nc1-c1nc(-c2ccc(N)nc2)ccc1[C@H](C)O ; Cc1nn(CC(F)(F)F)nc1-c1nc(-c2ccc(NC(=O)[C@H]3C[C@H]3c3ncc(C(F)F)cn3)nc2)ccc1[C@H](C)O]

Step 1. Synthesis of 1-(4-methoxybenzyl)-5-methyl-4-(tributylstannyl)-1H-1,2,3-triazole. Tributyl(prop-1-ynyl)stannane (30.0 g, 91.16 mmol, 1 eq) and 1-(azidomethyl)-4-methoxy-benzene (14.80 g, 91.16 mmol, 1 eq) was stirred at 180 0° C. for 15 minutes (Flow chemistry). The mixture was concentrated under reduced pressure to give a residue. The residue was purified by flash silica gel chromatography (Eluent of PE/EtOAc) to afford title compound. ¹H NMR (400 MHz, CD₃OD) δ (ppm): 7.09 (2H), 6.89-6.80 (2H), 5.45 (2H), 3.82-3.76 (3H), 2.16 (3H), 1.61-1.42 (6H), 1.38-1.29 (6H), 1.23-1.01 (6H), 0.87 (9H). LCMS: 494.2 [M+H]⁺.

Step 2. Synthesis of tert-butyl (S)-(6-chloro-5-(1-hydroxyethyl)-[2,3′-bipyridin]-6′-yl)carbamate. To a solution of (S)-1-(2,6-dichloropyridin-3-yl)ethan-1-ol (7.50 g, 39.05 mmol, 1 eq) and tert-butyl N-[5-(4,4,5,5-tetramethyl-1,3,2-dioxaborolan-2-yl)-2-pyridyl]carbamate (12.50 g, 39.05 mmol, 1 eq) in dioxane (75 mL) and H₂O (7.5 mL) was added K₃PO₄ (20.72 g, 97.63 mmol, 2.5 eq) and Pd(dppf)Cl₂—CH₂Cl₂ (3.2 g, 3.91 mmol, 0.1 eq) under N₂ atmosphere. The mixture was stuffed at 40° C. for 1 h. The mixture was diluted with H₂O (10 mL) and extracted with EtOAc (3×20 mL), the organic layer was washed with brine (10 ml), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, DMSO-d6) δ (ppm): 10.09 (1H), 8.92 (1H), 8.40-8.33 (1H), 8.05 (2H), 7.94 (1H), 5.57 (1H), 5.02-4.93 (1H), 1.49 (9H), 1.36 (3H). LCMS: 350.2 [M+H]⁺.

Step 3. Synthesis of (S)-1-(6′-amino-6-(1-(4-methoxybenzyl)-5-methyl-1H-1,2,3-triazol-4-yl)-[2,3′-bipyridin]-5-yl)ethan-1-ol. To a solution of tert-butyl N-[5-[6-chloro-5-[(1S)-1-hydroxyethyl]-2-pyridyl]-2-pyridyl]carbamate (3.00 g, 8.58 mmol, 1 eq) and tributyl-[1-[(4-methoxyphenyl)methyl]-5-methyl-triazol-4-yl]stannane (12.67 g, 25.73 mmol, 3 eq) in toluene (50 mL) was added Pd₂(dba)₃ (785 mg, 858 mol, 0.1 eq) and tris-o-tolylphosphane (1.04 g, 3.43 mmol, 0.4 eq). The mixture was stirred at 120° C. for 16 h. The reaction mixture was filtered and concentrated under reduced pressure to give a residue. The residue was purified by flash silica gel chromatography (Eluent of DCM/MeOH) and reverse phase preparative HPLC (ACN/H₂O, 0.05% NH₄OH) to afford the title compound. ¹H NMR (400 MHz, CD₃OD) δ (ppm): 8.59 (1H), 8.17-8.05 (2H), 7.77 (1H), 7.25 (2H), 6.94 (2H), 6.65 (1H), 5.61 (2H), 5.29 (1H), 3.78 (3H), 2.47 (3H), 1.47 (3H).

Step 4. Synthesis of tert-butyl (S)-(5-(1-hydroxyethyl)-6-(1-(4-methoxybenzyl)-5-methyl-1H-1,2,3-triazol-4-yl)-[2,3′-bipyridin]-6′-yl)carbamate. To a solution of (1S)-1-[6-(6-amino-3-pyridyl)-2-[1-[(4-methoxyphenyl)methyl]-5-methyl-triazol-4-yl]-3-pyridyl]ethanol (1.10 g, 2.64 mmol, 1 eq) in t-BuOH (50 mL) was added Boc₂O (867 mg, 3.96 mmol, 910 μL, 1.5 eq). The mixture was stirred at 40° C. for 16 h. The mixture was diluted with H₂O (10 mL) and extracted with EtOAc (3×20 mL), the organic layers was washed with brine (10 ml), dried over anhydrous Na₂SO₄, filtered and concentrated under reduced pressure to give a residue. The residue was purified by flash silica gel chromatography (Eluent of DCM/MeOH) to afford the title compound. ¹H NMR (400 MHz, CD₃OD) δ (ppm): 8.89 (1H), 8.41-8.27 (1H), 8.16 (1H), 7.95 (1H), 7.87 (1H), 7.25 (2H), 6.93 (2H), 5.60 (3H), 3.78 (3H), 2.48 (3H), 1.53 (9H), 1.49 (3H). LCMS: 517.3 [M+H]⁺.

Step 5. Synthesis of tert-butyl (S)-(5-(1-hydroxyethyl)-6-(5-methyl-1H-1,2,3-triazol-4-yl)-[2,3′-bipyridin]-6′-yl)carbamate. To a solution of tert-butyl N-[5-[5-[(1S)-1-hydroxyethyl]-6-[1-[(4-methoxyphenyl)methyl]-5-methyl-triazol-4-yl]-2-pyridyl]-2-pyridyl]carbamate (580 mg, 1.12 mmol, 1 eq) in ACN (12 mL) was added CAN (1.54 g, 2.81 mmol, 1.40 mL, 2.5 eq) in H₂O (12 mL) at 0° C. The mixture was stirred at 30° C. for 1 h. Then another batch of CAN (923 mg, 1.68 mmol, 839 μL, 1.5 eq) in H₂O (2 mL) was added to the mixture and stirred at 30° C. for another 0.5 h. The mixture was diluted with saturated NaHCO₃ aqueous solution (10 ml). The resulting mixture was filtered. The filter cake was washed with DCM (5 mL×2). The combined filtrate was extracted with DCM:MeOH=5:1 (15 ml×3). The combined organic layers were washed with saturated NaHCO₃ aqueous solution (10 ml) and dried over anhydrous Na₂SO₄, filtered and concentrated under reduced pressure to give a residue. The residue was purified by flash silica gel chromatography (Eluent of DCM/MeOH) to afford the title compound. ¹H NMR (400 MHz, CDCl₃) δ (ppm): 12.50-11.90 (1H), 8.98-8.94 (1H), 8.38-8.32 (1H), 8.08 (1H), 8.02 (1H), 7.75-7.66 (2H), 5.31 (1H), 5.19-5.09 (1H), 2.73 (3H), 1.63 (3H), 1.56 (9H). LCMS: 397.1 [M+H]⁺.

Step 6. Synthesis of tert-butyl (S)-(5-(1-hydroxyethyl)-6-(5-methyl-2-(2,2,2-trifluoroethyl)-2H-1,2,3-triazol-4-yl)-[2,3′-bipyridin]-6′-yl)carbamate & tert-butyl (S)-(5-(1-hydroxyethyl)-6-(5-methyl-1-(2,2,2-trifluoroethyl)-1H-1,2,3-triazol-4-yl)-[2,3′-bipyridin]-6′-yl)carbamate. To a solution of tert-butyl N-[5-[5-[(1S)-1-hydroxyethyl]-6-(5-methyl-1H-triazol-4-yl)-2-pyridyl]-2-pyridyl]carbamate (320 mg, 807.18 mol, 1 eq) in DMF (4 mL) was added Cs₂CO₃ (789 mg, 2.42 mmol, 3 eq) and 2,2,2-trifluoroethyl trifluoromethanesulfonate (187 mg, 807 mol, 1 eq). The mixture was stirred at 30° C. for 0.5 h. Then it was concentrated under reduced pressure to give a residue. The residue was purified by reverse phase preparative HPLC (ACN/H₂O, 0.05% NH₄OH) to afford two regioisomers:

Isomer 1 (tert-butyl (S)-(5-(1-hydroxyethyl)-6-(5-methyl-1-(2,2,2-trifluoroethyl)-1H-1,2,3-triazol-4-yl)-[2,3′-bipyridin]-6′-yl)carbamate): ¹H NMR (400 MHz, CDCl₃) δ (ppm): 8.93 (1H), 8.34-8.26 (1H), 8.10-7.99 (2H), 7.72 (1H), 7.66 (1H), 5.26 (1H), 5.20-5.10 (1H), 5.06-4.96 (2H), 2.77 (3H), 1.64 (3H), 1.55 (9H). LCMS: 479.2 [M+H]⁺.

Isomer 2 (tert-butyl (S)-(5-(1-hydroxyethyl)-6-(5-methyl-2-(2,2,2-trifluoroethyl)-2H-1,2,3-triazol-4-yl)-[2,3′-bipyridin]-6′-yl)carbamate): ¹H NMR (400 MHz, CDCl₃) δ (ppm): 8.97 (1H), 8.39-8.27 (1H), 8.14-8.00 (3H), 7.75 (1H), 5.22-5.10 (1H), 5.07-4.95 (2H), 3.86-3.69 (1H), 2.66 (3H), 1.59 (3H), 1.54 (9H). LCMS: 479.2 [M+H]⁺.

Step 7. Synthesis of (S)-1-(6′-amino-6-(5-methyl-1-(2,2,2-trifluoroethyl)-1H-1,2,3-triazol-4-yl)-[2,3′-bipyridin]-5-yl)ethan-1-ol. A solution of tert-butyl (S)-(5-(1-hydroxyethyl)-6-(5-methyl-1-(2,2,2-trifluoroethyl)-1H-1,2,3-triazol-4-yl)-[2,3′-bipyridin]-6′-yl)carbamate (45 mg, 94 mol, 1 eq, isomer 1 from step 6) in HCl/dioxane (2 M, 2 mL, 43 eq) was stirred at 25° C. for 16 h. The mixture was concentrated in vacuo. The residue was diluted with MeOH (2 mL) and then basified with NaHCO₃ until pH=8. The resulting mixture was filtered. The filtrate was concentrated under reduced pressure to afford the title compound, which was used into next step directly, without any further purification. LCMS: 379.1 [M+H]⁺.

Step 8. Synthesis of (1S,2R)-2-(5-(difluoromethyl)pyrimidin-2-yl)-N-(5-((S)-1-hydroxyethyl)-6-(5-methyl-1-(2,2,2-trifluoroethyl)-1H-1,2,3-triazol-4-yl)-[2,3′-bipyridin]-6′-yl)cyclopropane-i-carboxamide. To a solution of (1S)-1-[6-(6-amino-3-pyridyl)-2-[5-methyl-1-(2,2,2-trifluoroethyl)triazol-4-yl]-3-pyridyl]ethanol (35 mg, 93 mol, 1 eq) in THF (0.5 mL) was added AlMe₃ (2 M in toluene, 231 μL, 5 eq) at 0° C. under N₂ atmosphere. The mixture was stirred at 40° C. for 1 h. Ethyl (1S,2R)-2-[5-(difluoromethyl)pyrimidin-2-yl]cyclopropanecarboxylate (27 mg, 111 mol, 1.2 eq) in THF (0.5 mL) was added to the mixture at 20° C. under N₂ atmosphere. The resulting mixture was stirred at 90° C. for another 14 h. The mixture was cooled to room temperature, quenched with saturated NaHCO₃ aqueous solution (15 mL) under N₂ atmosphere. The resulting mixture was filtered. The filter cake was washed with DCM:MeOH=5:1 (2×5 mL). The combined filtrate was extracted with DCM:MeOH=5:1 (3×10 mL). The combined organic layer was washed with brine (10 mL), dried over anhydrous Na₂SO₄, filtered and concentrated under reduced pressure to give a residue. The residue was purified by reverse phase preparative HPLC (ACN/H₂O, 0.05% NH₄HCO₃) to afford the title compound. ¹H NMR (400 MHz, CD₃OD) δ (ppm): 8.96 (1H), 8.82 (2H), 8.38-8.28 (1H), 8.19 (1H), 7.99-7.86 (2H), 7.13-6.71 (1H), 5.44-5.36 (2H), 5.36-5.27 (1H), 2.99-2.89 (1H), 2.62 (3H), 2.59-2.53 (1H), 2.14-2.04 (1H), 1.65-1.55 (1H), 1.49 (3H). LCMS: 575.2 [M+H]⁺.

Step 9. Synthesis of ((S)-1-(6′-amino-6-(5-methyl-2-(2,2,2-trifluoroethyl)-2H-1,2,3-triazol-4-yl)-[2,3′-bipyridin]-5-yl)ethan-1-ol. A solution of tert-butyl (S)-(5-(1-hydroxyethyl)-6-(5-methyl-2-(2,2,2-trifluoroethyl)-2H-1,2,3-triazol-4-yl)-[2,3′-bipyridin]-6′-yl)carbamate (50 mg, 105 mol, 1 eq, isomer 2 from step 6) in HCl/dioxane (2 M, 2 mL, 38 eq) was stirred at 25° C. for 16 h. The mixture was concentrated in vacuo. The residue was diluted with MeOH (2 mL) and then basified with NaHCO₃ until pH=8. The resulting mixture was filtered. The filtrate was concentrated in vacuo to afford the title compound, which was used into next step directly, without any further purification. LCMS: 379.1 [M+H]⁺.

Step 10. Synthesis of (1S,2R)-2-(5-(difluoromethyl)pyrimidin-2-yl)-N-(5-((S)-1-hydroxyethyl)-6-(5-methyl-2-(2,2,2-trifluoroethyl)-2H-1,2,3-triazol-4-yl)-[2,3′-bipyridin]-6′-yl)cyclopropane-i-carboxamide. To a solution of (S)-1-(6′-amino-6-(5-methyl-2-(2,2,2-trifluoroethyl)-2H-1,2,3-triazol-4-yl)-[2,3′-bipyridin]-5-yl)ethan-1-ol (39 mg, 103 mol, 1 eq) in THF (0.5 mL) was added AlMe₃ (2 M in toluene, 258 μL, 5 eq) at 0° C. under N₂ atmosphere. The mixture was stirred at 40° C. for 1 h. Ethyl (1S, 2R)-2-[5-(difluoromethyl)pyrimidin-2-yl]cyclopropanecarboxylate (30 mg, 124 mol, 1.2 eq) in THF (0.5 mL) was added to the mixture at 20° C. under N₂ atmosphere. The resulting mixture was stirred at 90° C. for another 14 h. The mixture was cooled to room temperature, quenched with saturated NaHCO₃ aqueous solution (15 mL) under N₂ atmosphere. The resulting mixture was filtered. The filter cake was washed with DCM:MeOH=5:1 (2×5 mL). The combined filtrate was extracted with DCM:MeOH=5:1 (3×10 mL). The combined organic layer was washed with brine (10 mL), dried over anhydrous Na₂SO₄, filtered and concentrated under reduced pressure to give a residue. The residue was purified by reverse phase preparative HPLC (ACN/H₂O, 0.05% NH₄HCO₃) to afford the title compound. ¹H NMR (400 MHz, CD₃OD) δ (ppm): 8.98 (1H), 8.83 (2H), 8.39-8.30 (1H), 8.20 (1H), 7.93 (2H), 7.12-6.74 (1H), 5.44-5.36 (1H), 5.31-5.20 (2H), 3.01-2.90 (1H), 2.62-2.55 (1H), 2.54 (3H), 2.14-2.04 (1H), 1.66-1.53 (1H), 1.47 (3H). LCMS: 575.2 [M+H]⁺.

### Example 30. Synthesis of (1S,2R)—N-(5-(1,1-difluoroethyl)-6-(3-methyl-1-(2,2,2-trifluoroethyl)-1H-pyrazol-4-yl)-[2,3′-bipyridin]-6′-yl)-2-(5-(difluoromethyl)pyrimidin-2-yl)cyclopropane-1-carboxamide (I-161)

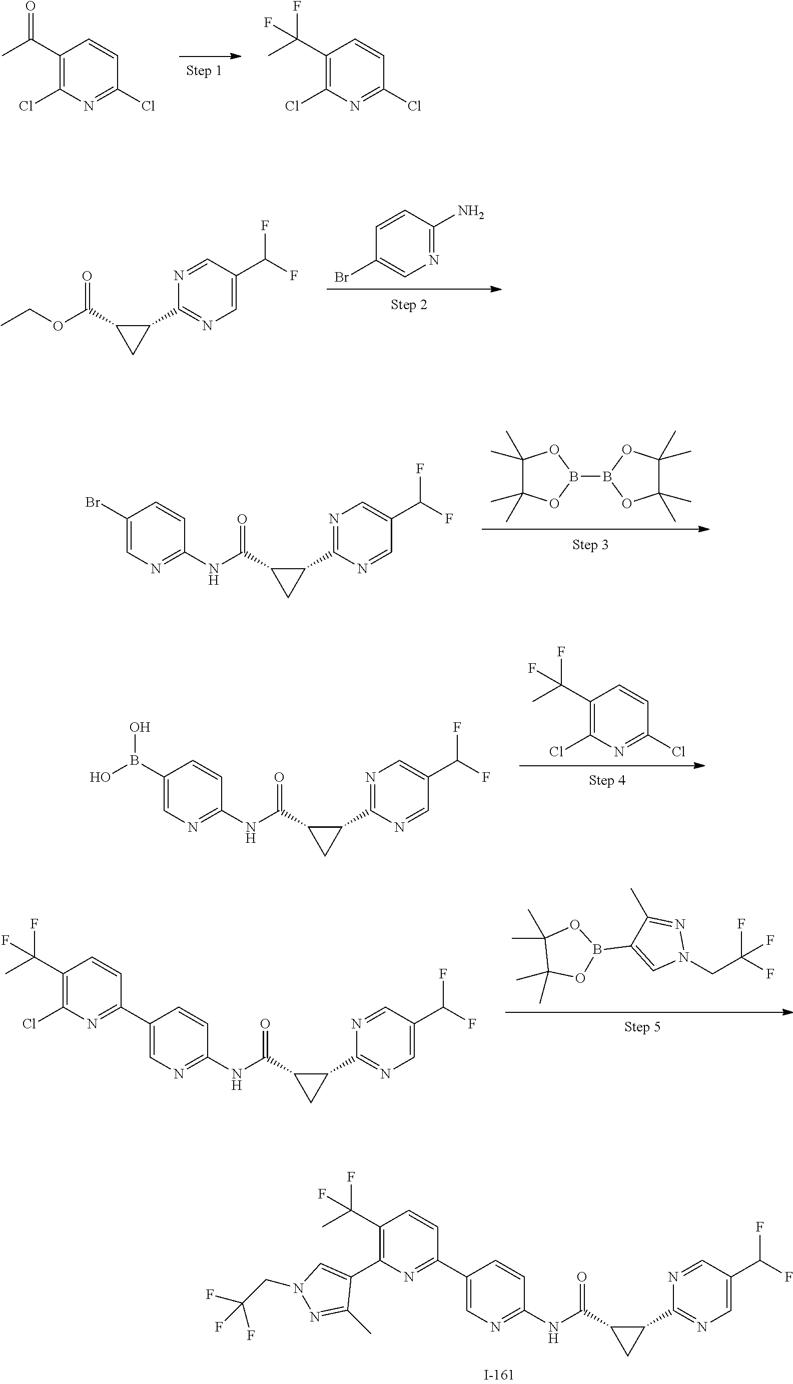

Step 1. Synthesis of 2,6-dichloro-3-(1,1-difluoroethyl)pyridine. To a solution of 1-(2,6-dichloropyridin-3-yl)ethan-1-one (500 mg, 2.63 mmol, 1 eq) was added DAST (5 mL). The mixture was stirred at 25° C. for 16 h. The mixture was quenched with saturated NaHCO₃ aqueous solution (10 mL) until pH=8. The resulting mixture was extracted with EtOAc (3×20 mL), the organic layer was washed with brine (10 ml), dried over anhydrous Na₂SO₄, filtered and concentrated under reduced pressure. The residue was purified by flash silica gel chromatography (Eluent of PE/EtOAc) to afford the title compound. ¹H NMR (400 MHz, CDCl₃) δ (ppm): 7.91 (1H), 7.37 (1H), 2.07 (3H). LCMS: 212.0 [M+H]⁺.

Step 2. Synthesis of (1S,2R)—N-(5-bromopyridin-2-yl)-2-(5-(difluoromethyl)pyrimidin-2-yl)cyclopropane-1-carboxamide. A mixture of ethyl (1S,2R)-2-[5-(difluoromethyl)pyrimidin-2-yl]cyclopropanecarboxylate (2.00 g, 8.26 mmol, 1 eq) and 5-bromopyridin-2-amine (1.43 g, 8.26 mmol, 1 eq) in toluene (30 mL) was degassed and purged with N₂ for 3 times and cooled to 0° C., then LiHMDS (1 M in THF, 16.51 mL, 2 eq) was added at 0° C. under N₂ atmosphere. The mixture was warmed to 40° C. and stirred at 40° C. for 1 h under N₂ atmosphere. The reaction mixture was quenched with saturated aqueous NH₄Cl (50 mL) and extracted with EtOAc (100 mL×3). The combined organic layers were washed with brine (100 mL×2), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, DMSO-d₆) δ (ppm): 10.94 (s, 1H), 8.87 (s, 2H), 8.38 (d, 1H), 7.91-7.81 (m, 1H), 7.73 (d, 1H), 7.15 (t, 1H), 2.94-2.82 (m, 1H), 2.59-2.53 (m, 1H), 1.91-1.81 (m, 1H), 1.52-1.42 (m, 1H). LCMS: 371.0 [M+H]⁺.

Step 3. Synthesis of (6-((1S,2R)-2-(5-(difluoromethyl)pyrimidin-2-yl)cyclopropane-1-carboxamido)pyridin-3-yl)boronic acid. A mixture of (1S,2R)—N-(5-bromo-2-pyridyl)-2-[5-(difluoromethyl)pyrimidin-2-yl]cyclopropanecarboxamide (480 mg, 1.30 mmol, 1 eq), 4,4,5,5-tetramethyl-2-(4,4,5,5-tetramethyl-1,3,2-dioxaborolan-2-yl)-1,3,2-dioxaborolane (495.27 mg, 1.95 mmol, 1.5 eq), Pd(dppf)Cl₂⋅DCM (106 mg, 130 mol, 0.1 eq) and KOAc (383 mg, 3.90 mmol, 3 eq) in dioxane (5 mL) was degassed and purged with N₂ for 3 times, and then the mixture was stirred at 80° C. for 2 h under N₂ atmosphere. The reaction solution was filtered through diatomite and the filtrate was concentrated under reduced pressure to give a crude product. The crude product was dissolved in DMF (2 mL) and purified by reverse phase preparative HPLC (H₂O (0.1% FA)-ACN) to afford the title compound. ¹H NMR (400 MHz, CD₃OD) δ (ppm): 8.81 (s, 2H), 8.54-8.34 (m, 1H), 8.05-7.94 (m, 1H), 7.76-7.62 (m, 1H), 6.91 (t, 1H), 3.03-2.86 (m, 1H), 2.62-2.49 (m, 1H), 2.13-2.03 (m, 1H), 1.64-1.52 (m, 1H). LCMS: 335.2 [M+H]⁺.

Step 4. Synthesis of (1S,2R)—N-(6-chloro-5-(1,1-difluoroethyl)-[2,3′-bipyridin]-6′-yl)-2-(5-(difluoromethyl)pyrimidin-2-yl)cyclopropane-1-carboxamide. To a solution of (6-((1S,2R)-2-(5-(difluoromethyl)pyrimidin-2-yl)cyclopropane-1-carboxamido)pyridin-3-yl)boronic acid (150 mg, 449 mol, 1 eq) and 2,6-dichloro-3-(1,1-difluoroethyl)pyridine (114 mg, 539 mol, 1.2 eq) in dioxane (2.5 mL) and H₂O (0.5 mL) was added K₃PO₄ (286 mg, 1.35 mmol, 3 eq), followed by Pd(dppf)Cl₂·CH₂Cl₂ (73 mg, 90 mol, 0.2 eq) at 25° C. under N₂ atmosphere. The mixture was stirred at 40° C. for 1 h. The mixture was cooled to room temperature and then concentrated under reduced pressure to give a residue. The residue was purified by flash silica gel chromatography (Eluent of PE/EtOAc) to afford the title compound. ¹H NMR (400 MHz, CDCl₃) δ (ppm): 9.04-8.88 (2H), 8.80 (2H), 8.31-8.21 (1H), 8.15 (1H), 7.99 (1H), 7.67 (1H), 6.93-6.54 (1H), 3.04-2.88 (1H), 2.49-2.34 (1H), 2.24-2.03 (4H), 1.76-1.66 (1H). LCMS: 466.1 [M+H]⁺.

Step 5. Synthesis of (1S,2R)—N-(5-(1,1-difluoroethyl)-6-(3-methyl-1-(2,2,2-trifluoroethyl)-1H-pyrazol-4-yl)-[2,3′-bipyridin]-6′-yl)-2-(5-(difluoromethyl)pyrimidin-2-yl)cyclopropane-1-carboxamide. To a solution of (1S,2R)—N-(6-chloro-5-(1,1-difluoroethyl)-[2,3′-bipyridin]-6′-yl)-2-(5-(difluoromethyl)pyrimidin-2-yl)cyclopropane-1-carboxamide (65 mg, 140 mol, 1 eq) and 3-methyl-4-(4,4,5,5-tetramethyl-1,3,2-dioxaborolan-2-yl)-1-(2,2,2-trifluoroethyl)-1H-pyrazole (121 mg, 419 mol, 3 eq) in dioxane (1 mL) and H₂O (0.2 mL) was added K₃PO₄ (89 mg, 419 mol, 3 eq), followed by Pd(dppf)Cl₂·CH₂Cl₂ (23 mg, 28 mol, 0.2 eq) at 25° C. under N₂ atmosphere. The mixture was stirred at 80° C. for 1 h. The mixture was cooled to room temperature and then concentrated under reduced pressure to give a residue. The residue was purified by flash silica gel chromatography (Eluent of PE/EtOAc) and reverse phase preparative HPLC (ACN/H₂O, 0.05% NH₄HCO₃) to afford the title compound. ¹H NMR (400 MHz, CD₃OD) δ (ppm): 8.96 (1H), 8.82 (2H), 8.36-8.31 (1H), 8.14 (1H), 7.94 (1H), 7.90 (1H), 7.86 (1H), 7.10-6.74 (1H), 4.98-4.90 (2H), 2.99-2.90 (1H), 2.62-2.53 (1H), 2.26 (3H), 2.13-2.05 (1H), 1.72 (3H), 1.63-1.55 (1H). LCMS: 594.2 [M+H]⁺.

### Example 31. Synthesis of (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 2) (I-162 & I-163)

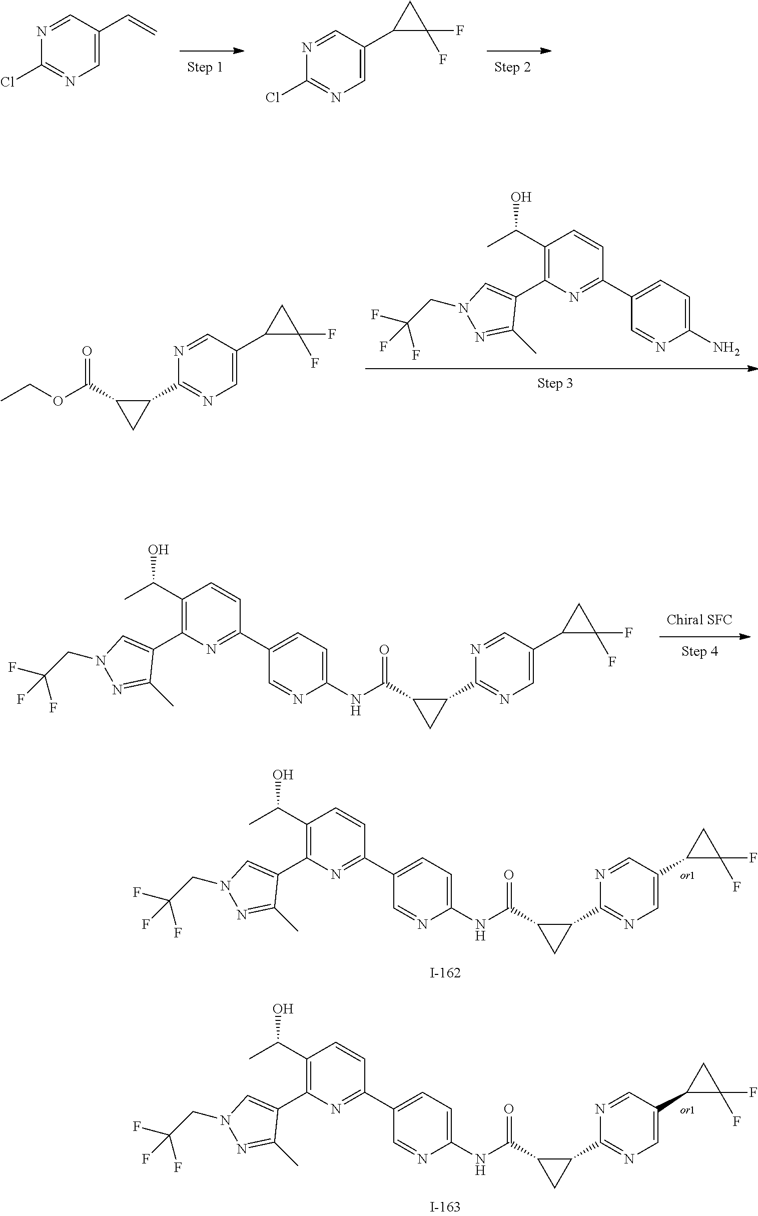

Step 1. Synthesis of 2-chloro-5-(2, 2-difluorocyclopropyl) pyrimidine. To a solution of 2-chloro-5-vinyl-pyrimidine (4.40 g, 31.30 mmol, 1 eq) and TBAB (605 mg, 1.88 mmol, 0.06 eq) in toluene (80 mL) was added TMSCF₂Br (19.00 g, 93.90 mmol, 3 eq). The mixture was stirred at 110° C. for 1 h. The reaction mixture was filtered and concentrated under reduced pressure to give a residue. The residue was purified by flash silica gel chromatography (Eluent of EtOAc/PE) to afford the title compound. ¹H NMR (400 MHz, CDCl₃) δ (ppm): 8.51 (1H), 8.44 (1H), 2.76-2.60 (1H), 2.10-1.96 (1H), 1.74-1.61 (1H).

Step 2. Synthesis of ethyl (1S,2R)-2-(5-(2,2-difluorocyclopropyl)pyrimidin-2-yl)cyclopropane-1-carboxylate. To a solution of 2-chloro-5-(2,2-difluorocyclopropyl)pyrimidine (3.00 g, 15.74 mmol, 1 eq) and ethyl (1S,2R)-2-(4,4,5,5-tetramethyl-1,3,2-dioxaborolan-2-yl)cyclopropanecarboxylate (4.20 g, 17.32 mmol, 1.1 eq) in dioxane (30 mL) and H₂O (7.5 mL) was added Cs₂CO₃ (15.40 g, 47.22 mmol, 3 eq), Pd(OAc)₂ (1.10 g, 4.72 mmol, 0.3 eq) and bis(1-adamantyl)-butyl-phosphane (CataCXium, 2.30 g, 6.30 mmol, 0.4 eq) under N₂ atmosphere. The mixture was stirred at 100° C. for 2 h. The reaction mixture was filtered and concentrated under reduced pressure to give a residue. The residue was purified by flash silica gel chromatography (Eluent of EtOAc/PE) to afford the title compound. ¹H NMR (400 MHz, CD₃OD) δ (ppm): 8.59 (2H), 3.90 (2H), 2.99-2.76 (2H), 2.32-2.20 (1H), 2.07-1.86 (3H), 1.53-1.44 (1H), 1.02 (3H). LCMS: 269.0 [M+H]⁺.

Step 3. Synthesis of (1S,2R)-2-(5-(2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a solution of AlMe₃ (2 M in toluene, 2.65 mL, 5.30 mmol, 5 eq) was added dropwise another solution of (S)-1-(6′-amino-6-(3-methyl-1-(2,2,2-trifluoroethyl)-1H-pyrazol-4-yl)-[2,3′-bipyridin]-5-yl)ethan-1-ol (400 mg, 1.06 mmol, 1 eq) and ethyl (1S,2R)-2-[5-(2,2-difluorocyclopropyl)pyrimidin-2-yl]cyclopropanecarboxylate (341 mg, 1.27 mmol, 1.2 eq) in dioxane (5 mL) at 90° C. under N₂ atmosphere. The resulting mixture was stirred at 90° C. for 1 h. The mixture was cooled to 0° C., quenched with saturated NH₄Cl aqueous solution (10 mL) under N₂ atmosphere and extracted with EtOAc (3×10 mL), the combined organic layer was washed with brine (10 ml), dried over anhydrous Na₂SO₄, filtered and concentrated under reduced pressure to give a residue. The residue was purified by reverse phase preparative HPLC (ACN/H₂O, 0.225% FA). Then the fraction was concentrated to remove acetonitrile and purified by reverse phase preparative HPLC (ACN/H₂O, 0.05% NH₄OH) to afford the title compound. LCMS: 600.2 [M+H]⁺.

Step 4. Synthesis of (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 2). (1S,2R)-2-(5-(2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide was separated by chiral SFC (SFC Preparative Method: Instrument: Thar 80Q Preparative SFC system; Column: DAICEL CHIRALPAK AD, 250×25 mm I.D., 10 m particle size; Mobilephase A: CO₂, Mobile Phase B: EtOH (0.1% NH₄OH); Isocratic elution: 55% Phase B in Supercritical CO₂; Flow rate: 75g/min; Retention time: Peak1: 3.13 min, Peak2: 4.18 min; Back Pressure: 100 bar to keep the CO₂ in Supercritical flow; Wave Length: 220 nm) to give the title compounds as peak 1 (Isomer 1) and peak 2 (Isomer 2).

Peak 1: (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 1) ¹H NMR (400 MHz, CD₃OD) δ (ppm): 8.91 (1H), 8.57 (2H), 8.32-8.24 (1H), 8.11 (1H), 7.93 (1H), 7.91 (1H), 7.83 (1H), 5.00-4.92 (3H), 2.95-2.82 (2H), 2.56-2.47 (1H), 2.33 (3H), 2.07-2.01 (1H), 2.00-1.91 (1H), 1.91-1.82 (1H), 1.60-1.50 (1H), 1.42 (3H). LCMS: 600.3 [M+H]⁺. Chiral SFC: Rt=1.782 min (instrument: SHIMADZU LC-30ADsf; column: Chiralpak AD-3 50×4.6 mm I.D., particle size 3 μm; mobile phase A: CO₂; mobile phase B: EtOH (0.05% DEA); 3 mL/min; 35° C., 10 MPa, 220 nm).

Peak 2: (1S,2R)-2-(5-((R*)-2,2-difluorocyclopropyl)pyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (Isomer 2) ¹H NMR (400 MHz, CD₃OD) δ (ppm): 8.91 (1H), 8.57 (2H), 8.30-8.25 (1H), 8.11 (1H), 7.93 (1H), 7.91 (1H), 7.83 (1H), 4.95-4.88 (3H), 2.94-2.82 (2H), 2.56-2.47 (1H), 2.33 (3H), 2.07-2.00 (1H), 2.00-1.91 (1H), 1.91-1.82 (1H), 1.59-1.51 (1H), 1.42 (3H). LCMS: 600.3 [M+H]⁺. Chiral SFC: Rt=2.003 min (instrument: SHIMADZU LC-30ADsf; column: Chiralpak AD-3 50×4.6 mm I.D., particle size 3 μm; mobile phase A: CO₂; mobile phase B: EtOH (0.05% DEA); 3 mL/min; 35° C., 10 MPa, 220 nm).

### Example 32. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(3-fluoroazetidin-1-yl)pyridin-2-yl)cyclopropane-1-carboxamide (I-164)

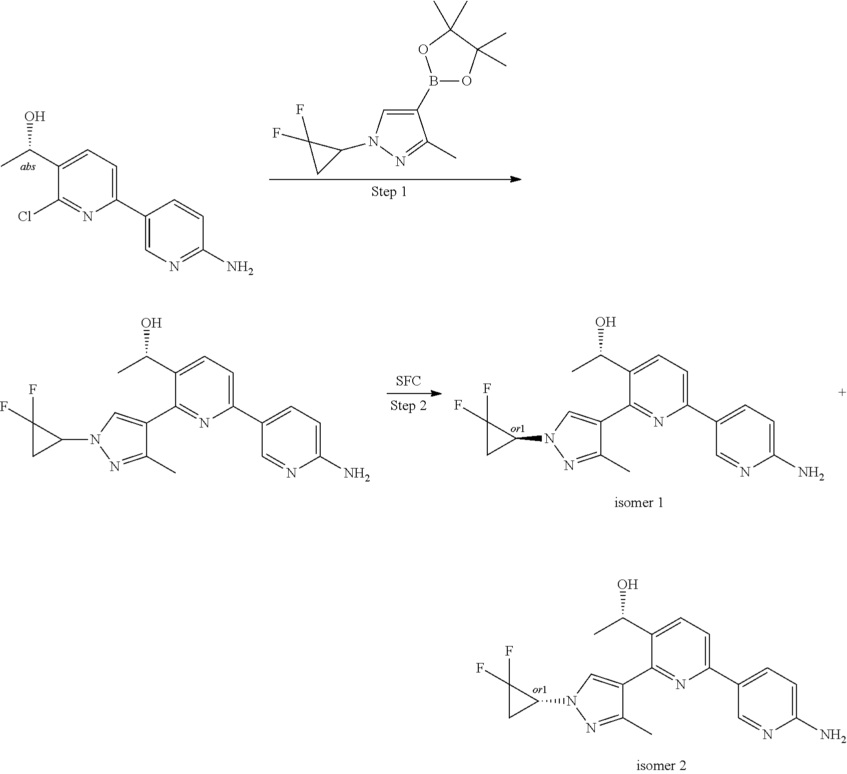 [SMILES: C[C@H](O)c1ccc(-c2ccc(N)nc2)nc1Cl ; Cc1nn(C2CC2(F)F)cc1B1OC(C)(C)C(C)(C)O1 ; Cc1nn(C2CC2(F)F)cc1-c1nc(-c2ccc(N)nc2)ccc1[C@H](C)O ; "SFC" ; Cc1nn([C@H]2CC2(F)F)cc1-c1nc(-c2ccc(N)nc2)ccc1[C@H](C)O ; Cc1nn([C@@H]2CC2(F)F)cc1-c1nc(-c2ccc(N)nc2)ccc1[C@H](C)O]

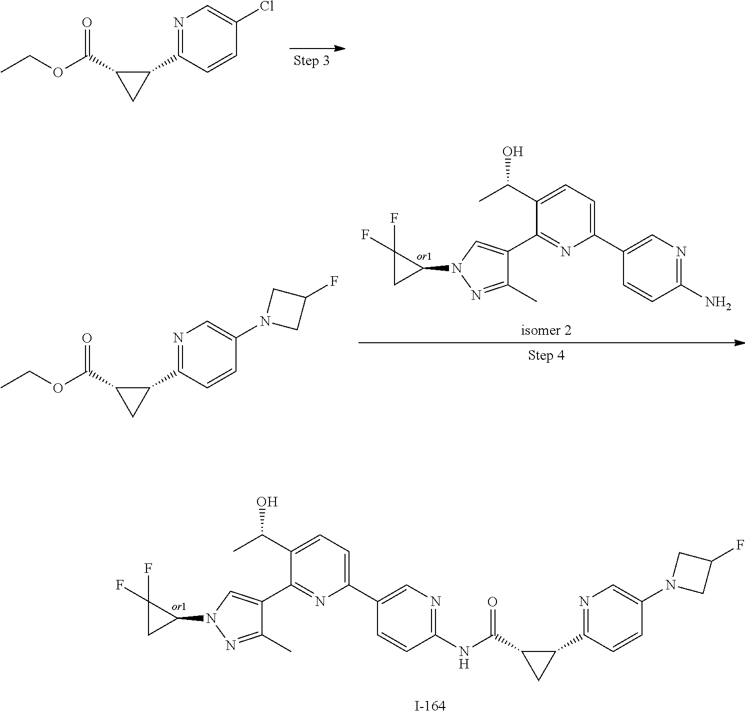 [SMILES: CCOC(=O)[C@H]1C[C@H]1c1ccc(Cl)cn1 ; CCOC(=O)[C@H]1C[C@H]1c1ccc(N2CC(F)C2)cn1 ; Cc1nn([C@H]2CC2(F)F)cc1-c1nc(-c2ccc(N)nc2)ccc1[C@H](C)O ; Cc1nn([C@H]2CC2(F)F)cc1-c1nc(-c2ccc(NC(=O)[C@H]3C[C@H]3c3ccc(N4CC(F)C4)cn3)nc2)ccc1[C@H](C)O]

Step 1. Synthesis of (1S)-1-(6′-amino-6-(1-(2,2-difluorocyclopropyl)-3-methyl-1H-pyrazol-4-yl)-[2,3′-bipyridin]-5-yl)ethan-1-ol. To a mixture of (S)-1-(6′-amino-6-chloro-[2,3′-bipyridin]-5-yl)ethan-1-ol (14.9 g, 59.60 mmol, 1 eq), 1-(2,2-difluorocyclopropyl)-3-methyl-4-(4,4,5,5-tetramethyl-1,3,2-dioxaborolan-2-yl)-1H-pyrazole (25.4 g, 89.40 mmol, 1.5 eq), K₃PO₄ (38.0 g, 178.80 mmol, 3 eq) in dioxane (300 mL) and H₂O (60 mL) was added Pd(dppf)Cl₂·CH₂Cl₂ (4.9 g, 5.96 mmol, 0.1 eq). The mixture was degassed and purged with N₂ for 3 times and then stirred at 80° C. for 1 h under N₂ atmosphere. The reaction mixture was diluted with water (500 mL) at 20° C. and extracted with EtOAc (300 mL×3). The combined organic layers were washed with brine (400 mL), dried over anhydrous Na₂SO₄, filtered and concentrated under reduced pressure to give a residue. The residue was purified by flash silica gel chromatography (Eluent of PE/EtOAc) to give the title compound. ¹H NMR (400 MHz, CD₃OD) δ (ppm): 8.56 (s, 1H), 8.14-8.07 (m, 1H), 8.05 (d, 1H), 7.88 (d, 1H), 7.72 (d, 1H), 6.66 (d, 1H), 4.97-4.89 (m, 1H), 4.37-4.27 (m, 1H), 2.37-2.16 (m, 5H), 1.41 (d, 3H). LCMS: 372.2 [M+H]⁺.

Step 2. Separation of (S)-1-(6′-amino-6-(1-(2,2-difluorocyclopropyl)-3-methyl-1H-pyrazol-4-yl)-[2,3′-bipyridin]-5-yl)ethan-1-ol (isomer 1) and (S)-1-(6′-amino-6-(1-(2,2-difluorocyclopropyl)-3-methyl-1H-pyrazol-4-yl)-[2,3′-bipyridin]-5-yl)ethan-1-ol (isomer 2). (1S)-1-(6′-amino-6-(1-(2,2-difluorocyclopropyl)-3-methyl-1H-pyrazol-4-yl)-[2,3′-bipyridin]-5-yl)ethan-1-ol was separated by chiral SFC (SFC Preparative Method: Instrument: Waters 350 Preparative SFC system; Column: DAICEL CHIRALPAK AD 250×50m, 10 m particle size; Mobilephase A: CO₂, Mobile Phase B: IPA:ACN (0.1% NH₄OH); Isocratic elution: 50% Phase B in Supercritical CO₂; Flow rate: 200 g/min; Retention time: Peak1: 5.32 min, Peak2: 8.77 min; Back Pressure: 80 bar to keep the CO₂ in Supercritical flow; Wave Length: 220 nm) to give Peak 1 as isomer 1 and Peak 2 as isomer 2.

Isomer 1 (Peak 1): ¹H NMR (400 MHz, CD₃OD) δ (ppm): 8.56 (s, 1H), 8.13-8.08 (m, 1H), 8.05 (d, 1H), 7.88 (s, 1H), 7.73 (d, 1H), 6.66 (d, 1H), 4.95-4.89 (m, 1H), 4.37-4.27 (m, 1H), 2.39-2.15 (m, 5H), 1.41 (d, 3H). LCMS: 372.2 [M+H]⁺

Isomer 2 (Peak 2): ¹H NMR (400 MHz, CD₃OD) δ=8.56 (s, 1H), 8.13-8.08 (m, 1H), 8.05 (d, 1H), 7.88 (s, 1H), 7.73 (d, 1H), 6.66 (d, 1H), 4.97-4.90 (m, 1H), 4.38-4.28 (m, 1H), 2.36-2.18 (m, 5H), 1.40 (d, 3H). LCMS: 372.2 [M+H]⁺

Step 3. Synthesis of ethyl (1S,2R)-2-(5-(3-fluoroazetidin-1-yl)pyridin-2-yl)cyclopropane-1-carboxylate. To a solution of ethyl (1S,2R)-2-(5-chloro-2-pyridyl)cyclopropanecarboxylate (1.00 g, 4.43 mmol, 1.0 eq) (prepared from 2-bromo-5-chloropyridine by analogy to step 1 of example 36) and 3-fluoroazetidine; hydrochloride (1.48 g, 13.29 mmol, 3.0 eq) in dioxane (20 mL) were added Cs₂CO₃ (4.33 g, 13.29 mmol, 3 eq) and Xphos Pd G4 (381 mg, 443 mol, 0.1 eq), the mixture was stirred at 100° C. for 1 h under N₂ atmosphere. The mixture was filtered and concentrated under treduced pressure. The residue was purified by flash silica gel chromatography (Eluent of EtOAc/PE) to afford the title compound. LCMS: 265.2 [M+H]⁺.

Step 4. Synthesis of (1S,2R)—N-(6-(1-((R*)-2,2-difluorocyclopropyl)-3-methyl-1H-pyrazol-4-yl)-5-((S)-1-hydroxyethyl)-[2,3′-bipyridin]-6′-yl)-2-(5-(3-fluoroazetidin-1-yl)pyridin-2-yl)cyclopropane-1-carboxamide. To a solution of AlMe₃ (2 M in toluene, 202 μL, 5.0 eq) was added dropwise another solution of (S)-1-(6′-amino-6-(1-((R*)-2,2-difluorocyclopropyl)-3-methyl-1H-pyrazol-4-yl)-[2,3′-bipyridin]-5-yl)ethan-1-ol (isomer 2 obtained in step 2) (30 mg, 81 mol, 1.0 eq) and ethyl (1S,2R)-2-(5-(3-fluoroazetidin-1-yl)pyridin-2-yl)cyclopropane-1-carboxylate (21 mg, 81 mol, 1.0 eq) in dioxane (1.0 mL) at 90° C. under N₂ atmosphere. The mixture was stirred at 90° C. for 12 h. The reaction mixture was diluted with saturated NH₄Cl solution (10 mL) and extracted with EtOAc (15 mL×3). The combined organic layers were washed with brine (15 mL), dried over anhydrous Na₂SO₄, filtered and concentrated under reduced pressure to give a residue. The residue was purified by reversed phase preparative HPLC (ACN/H₂O, 0.05% NH₄HCO₃) to afford the title compound. ¹H NMR (400 MHz, CD₃OD) δ (ppm): 8.90 (1H), 8.31-8.24 (1H), 8.11 (1H), 7.95-7.89 (2H), 7.83 (1H), 7.67 (1H), 7.23 (1H), 6.93-6.83 (1H), 5.54-5.29 (1H), 4.96 (1H), 4.39-4.29 (1H), 4.27-4.14 (2H), 4.02-3.87 (2H), 2.74-2.61 (1H), 2.43-2.18 (6H), 1.86-1.78 (1H), 1.50-1.44 (1H), 1.43-1.37 (3H). LCMS: 590.3 [M+H]⁺.

### Example 33. Synthesis of (1S,2R)—N-[5-[5-[(1S)-1-hydroxyethyl]-6-[3-methyl-1-(2,2,2-trifluoroethyl)pyrazol-4-yl]-2-pyridyl]-2-pyridyl]-2-[5-(1-hydroxy-1-methyl-ethyl)pyrazin-2-yl]cyclopropanecarboxamide (I-165)

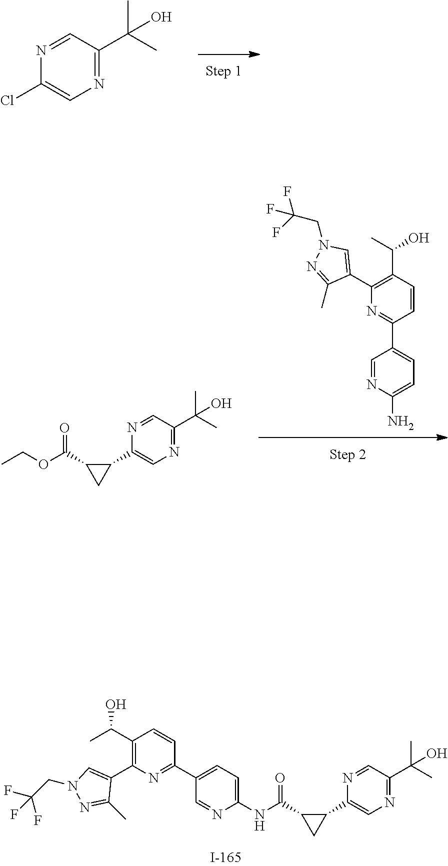 [SMILES: CC(C)(O)c1cnc(Cl)cn1 ; CCOC(=O)[C@H]1C[C@H]1c1cnc(C(C)(C)O)cn1 ; Cc1nn(CC(F)(F)F)cc1-c1nc(-c2ccc(N)nc2)ccc1[C@H](C)O ; Cc1nn(CC(F)(F)F)cc1-c1nc(-c2ccc(NC(=O)[C@H]3C[C@H]3c3cnc(C(C)(C)O)cn3)nc2)ccc1[C@H](C)O]

Step 1. Synthesis of ethyl (1S,2R)-2-(5-(2-hydroxypropan-2-yl)pyrazin-2-yl)cyclopropane-1-carboxylate. To a solution of 2-(5-chloropyrazin-2-yl)propan-2-ol (200 mg, 1.16 mmol, 1.0 eq) and ethyl (1S,2R)-2-(4,4,5,5-tetramethyl-1,3,2-dioxaborolan-2-yl)cyclopropanecarboxylate (334 mg, 1.39 mmol, 1.2 eq) in dioxane (4.00 mL) and H₂O (0.40 mL) was added Pd(OAc)₂ (52 mg, 232 mol, 0.2 eq), Catacxium A (125 mg, 347 mol, 0.3 eq) and Cs₂CO₃ (944 mg, 2.90 mmol, 2.5 eq). The mixture was stirred at 100° C. for 2 h. The reaction mixture was diluted with H₂O (10 mL) and extracted with EtOAc (10 mL×2). The combined organic layers were washed with brine (10 mL×2), dried over anhydrous Na₂SO₄, filtered and concentrated under reduced pressure to give a residue. The residue was purified by flash silica gel chromatography (Eluent of EtOAc/PE) to afford the title compound. LCMS: 251.1 [M+H]⁺.

Step 2. Synthesis of (1S,2R)—N-[5-[5-[(1S)-1-hydroxyethyl]-6-[3-methyl-1-(2,2,2-trifluoroethyl)pyrazol-4-yl]-2-pyridyl]-2-pyridyl]-2-[5-(1-hydroxy-1-methyl-ethyl)pyrazin-2-yl]cyclopropanecarboxamide. To a solution of AlMe₃ (2.0 M in toluene, 530 μL, 4.0 eq) in dioxane (4.00 mL) was added dropwise another solution of (1S)-1-[6-(6-amino-3-pyridyl)-2-[3-methyl-1-(2,2,2-trifluoroethyl)pyrazol-4-yl]-3-pyridyl]ethanol (100.00 mg, 265 mol, 1.0 eq) and ethyl (1S,2R)-2-[5-(1-hydroxy-1-methyl-ethyl)pyrazin-2-yl]cyclopropanecarboxylate (72.96 mg, 292 mol, 1.1 eq) in dioxane (4.00 mL) at 90° C. under N₂ atmosphere. The resulting mixture was stirred at 90° C. for 12 h. The reaction mixture was quenched by addition of saturated NH₄Cl aqueous solution (8 mL) at 25° C., and then diluted with H₂O (8 mL) and extracted with EtOAc (4 mL×3). The combined organic layers were washed with brine (5 mL×2), dried over anhydrous Na₂SO₄, filtered and concentrated under reduced pressure to give a residue. The residue was purified by reversed phase preparative HPLC (ACN/H₂O, 0.05% NH₄HCO₃) to afford the title compound. ¹H NMR (400 MHz, CD₃OD) δ (ppm): 8.92 (1H), 8.73 (1H), 8.55 (1H), 8.31-8.25 (1H), 8.12 (1H), 7.94 (1H), 7.89 (1H), 7.84 (1H), 4.99-4.95 (3H), 2.78 (1H), 2.53 (1H), 2.33 (3H), 1.98 (1H), 1.58 (1H), 1.53 (6H), 1.42 (3H). LCMS: 582.3 [M+H]⁺.

### Example 34. Synthesis of (1S,2R)-2-(4-(difluoromethyl)-5-methylpyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide (I-66)

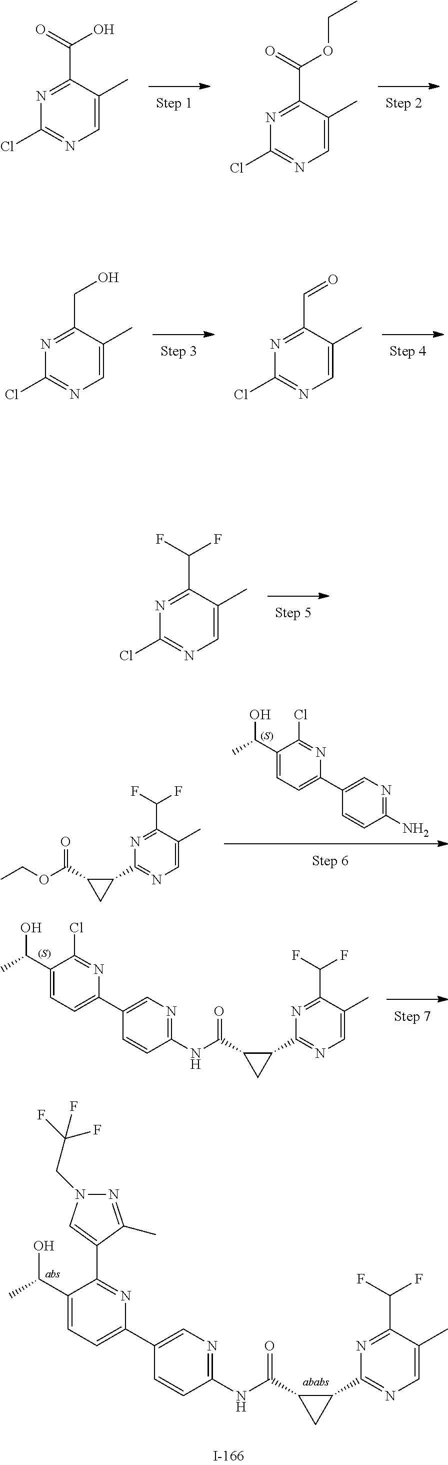

Step 1. Synthesis of Ethyl 2-chloro-5-methylpyrimidine-4-carboxylate. To a stirred solution of 2-chloro-5-methylpyrimidine-4-carboxylic acid (8 g, 46.3 mmol) in EtOH (200 mL) were added SOCl₂ (11 g, 92.7 mmol) dropwise at 0° C. under N₂ atmosphere. The resulting mixture was stirred at RT for 2 h under N₂ atmosphere. The reaction was quenched by the addition of water (600 mL) at 0° C. and extracted with CH₂Cl₂ (3×300 mL). The combined organic layers were dried over anhydrous Na₂SO₄, filtered, and concentrated under reduced pressure. The residue was purified by silica gel column chromatography (Petroleum ether/EtOAc 5:1) to afford the title product. LCMS: 201 [M+H]⁺.

Step 2. Synthesis of (2-chloro-5-methylpyrimidin-4-yl)methanol. To a stirred solution of ethyl 2-chloro-5-methylpyrimidine-4-carboxylate (7 g, 34.8 mmol) in CH₂Cl₂ (200 mL) were added diisobutylaluminum hydride (1.0 M in THF, 104 mL, 104 mmol) dropwise at 0° C. under N₂ atmosphere and the mixture was stirred at 0° C. for 1 h under N₂ atmosphere. The reaction mixture was concentrated under reduced pressure. The residue was purified by Prep-TLC (CH₂Cl₂/MeOH 10:1) to afford the title product. LCMS: 159 [M+H]⁺.

Step 3. Synthesis of 2-chloro-5-methylpyrimidine-4-carbaldehyde. To a stirred solution of 2-chloro-5-methylpyrimidin-4-yl)methanol (2 g, 12.6 mmol) in CH₂Cl₂ (20 mL) was added DMP (10.7 g, 25.2 mmol) in portions at 0° C. and the mixture was stirred at RT for 1 h. The resulting mixture was filtered and the filter cake was washed with CH₂Cl₂ (2×2 mL). The filtrate was concentrated under reduced pressure. The residue was purified by Prep-TLC (Petroleum ether/EtOAc 3:1) to afford the title product. LCMS: 157 [M+H]⁺.

Step 4. Synthesis of 2-chloro-4-(difluoromethyl)-5-methylpyrimidine. To a stirred solution of 2-chloro-5-methylpyrimidine-4-carbaldehyde (560 mg, 3.57 mmol) in CH₂Cl₂ (3 mL) were added a solution of DAST (1.7 g, 10.7 mmol) dropwise at 0° C. under N₂ atmosphere, stirred at RT for 30 min under N₂ atmosphere. The reaction was quenched with water at 0° C. The resulting mixture was concentrated under reduced pressure. The residue was purified by Prep-TLC (Petroleum ether/EtOAc 2:1) to afford the title product. LCMS: 179 [M+H]⁺.

Step 5. Synthesis of ethyl (1S,2R)-2-(4-(difluoromethyl)-5-methylpyrimidin-2-yl)cyclopropane-1-carboxylate. To a stirred solution of 2-chloro-4-(difluoromethyl)-5-methylpyrimidine (220 mg, 1.23 mmol) and ethyl (1S,2R)-2-(4,4,5,5-tetramethyl-1,3,2-dioxaborolan-2-yl)cyclopropane-1-carboxylate (591 mg, 2.46 mmol) in toluene (3 mL) were added Cs₂CO₃ (1.2 g, 3.69 mmol), CataCXiumA (88 mg, 0.246 mmol) and Pd(OAc)₂ (27 mg, 0.123 mmol) at RT under N₂ atmosphere and the mixture was stirred at 100° C. for 1 h under N₂ atmosphere. The resulting mixture was concentrated under reduced pressure. The residue was purified by Prep-TLC (Petroleum ether/EtOAc 2:1) to afford the title product. LCMS: 257 [M+H]⁺.

Step 6. Synthesis of (1S,2R)—N-(6-chloro-5-((S)-1-hydroxyethyl)-[2,3′-bipyridin]-6′-yl)-2-(4-(difluoromethyl)-5-methylpyrimidin-2-yl)cyclopropane-1-carboxamide. To a stirred solution of ethyl (1S,2R)-2-(4-(difluoromethyl)-5-methylpyrimidin-2-yl)cyclopropane-1-carboxylate (138 mg, 0.54 mmol) and (S)-1-(6′-amino-6-chloro-[2,3′-bipyridin]-5-yl)ethan-1-ol (45 mg, 0.18 mmol) in THF (6 mL) were added LiHMDS (1.0 M in THF, 0.6 mL, 0.60 mmol) dropwise at RT under N₂ atmosphere and the mixture was stirred at RT for 2 h under N₂ atmosphere. The resulting mixture was concentrated under reduced pressure. The residue was purified by Prep-TLC (CH₂Cl₂/MeOH 10:1) to afford the title product. LCMS: 460 [M+H]⁺.

Step 7. Synthesis of (1S,2R)-2-(4-(difluoromethyl)-5-methylpyrimidin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a stirred solution of (1S,2R)—N-(6-chloro-5-((S)-1-hydroxyethyl)-[2,3′-bipyridin]-6′-yl)-2-(4-(difluoromethyl)-5-methylpyrimidin-2-yl) cyclopropane-1-carboxamide (35 mg, 0.076 mmol) and 3-methyl-4-(4,4,5,5-tetramethyl-1,3,2-dioxaborolan-2-yl)-1-(2,2,2-trifluoroethyl)-1H-pyrazole (331 mg, 1.14 mmol) in Dioxane (1.6 mL) were added K₂CO₃ (31 mg, 0.22 mmol), water (0.8 mL) and Pd(PPh₃)₄(17 mg, 0.015 mmol) at RT under N₂ atmosphere and the mixture was stirred at 100° C. for 1 h under N₂ atmosphere. The resulting mixture was concentrated under vacuum. The residue was purified by Prep-TLC (CH₂Cl₂/MeOH 15:1) to afford the crude product. The crude product was purified by Chiral-HPLC (Column: JW-CHIRALPAK IK, 20×250 mm, 5 μm; Mobile Phase A: EtOH:DCM=1:1 HPLC, Mobile Phase B: Hex (0.5% 2 M NH₃-MeOH)—HPLC; Flow rate: 20 mL/min; Gradient (B %): 70% B to 70% B in 12 min; Wave Length: 220/254 nm; RT1(min): 6.53; Sample Solvent: EtOH:DCM=1:1-HPLC; Injection Volume: 1.3 mL; Number of Runs: 2). The desired fractions were concentrated under reduced pressure. The residue was dissolved in ACN and H₂O and lyophilized to provide the title compound. LCMS: 588 [M+H]⁺. ¹H NMR (400 MHz, DMSO-d6) δ (ppm): 10.94 (1H), 9.00 (1H), 8.71 (1H), 8.36-8.32 (1H), 8.06-8.03 (2H), 7.92 (1H), 7.86 (1H), 6.97 (1H), 5.40-5.28 (1H), 5.17-5.11 (2H), 4.89-4.82 (1H), 2.88-2.82 (1H), 2.59-2.53 (1H), 2.34 (3H), 2.29 (3H), 1.88-1.84 (1H), 1.48-1.43 (1H), 1.33 (3H).

### Example 35. Synthesis of 1-(5-((S)-1-hydroxyethyl)-6-(3-methyl-1-(2,2,2-trifluoroethyl)-1H-pyrazol-4-yl)-[2,3′-bipyridin]-6′-yl)-3-((R*)-1-(5-methylpyridin-2-yl)ethyl)urea (Isomer 1) & 1-(5-((S)-1-hydroxyethyl)-6-(3-methyl-1-(2,2,2-trifluoroethyl)-1H-pyrazol-4-yl)-[2,3′-bipyridin]-6′-yl)-3-((S*)-1-(5-methylpyridin-2-yl)ethyl)urea (Isomer 2) (I-167 & I-168)

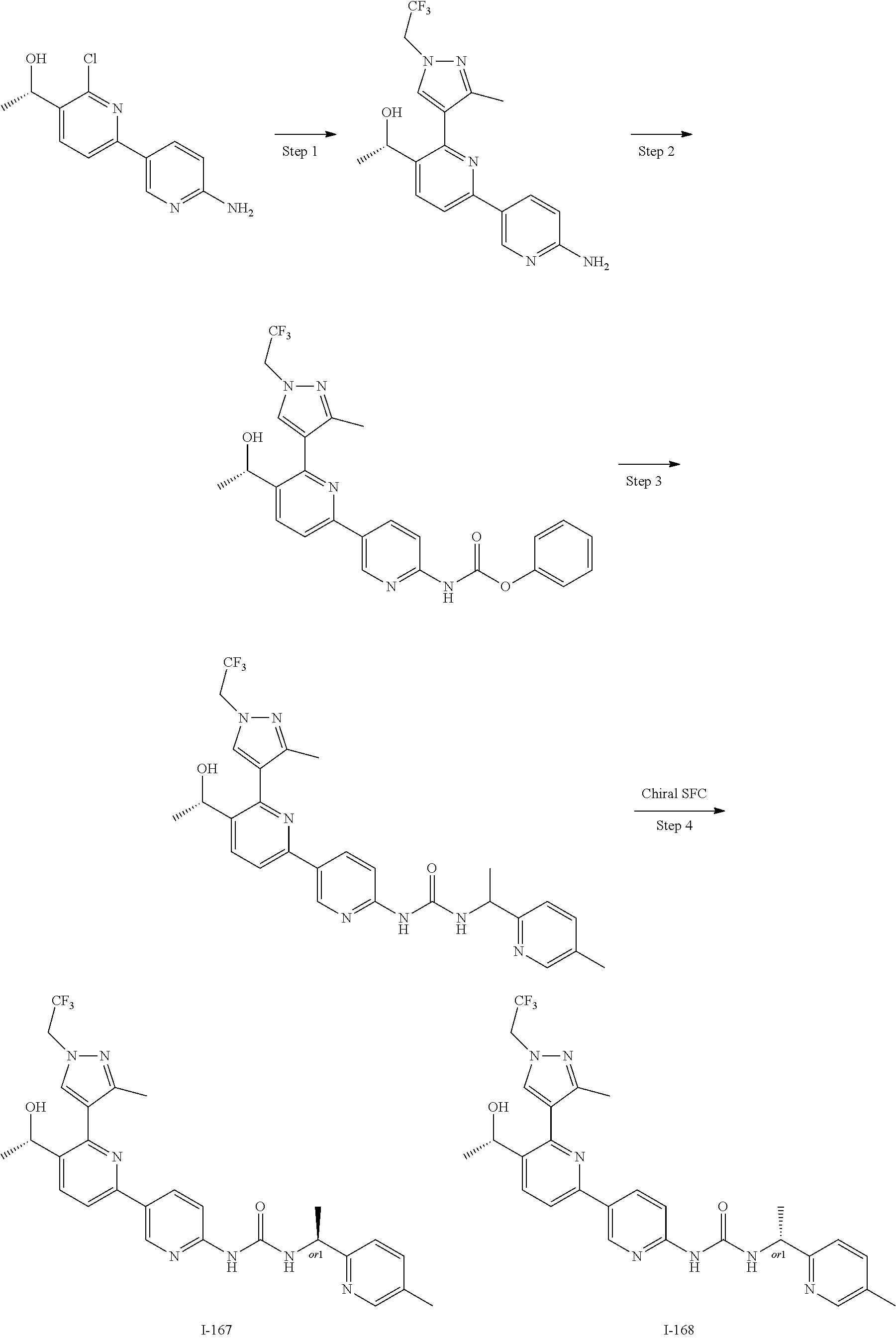

Step 1. Synthesis of (S)-1-(6′-amino-6-(3-methyl-1-(2,2,2-trifluoroethyl)-1H-pyrazol-4-yl)-[2,3′-bipyridin]-5-yl) ethan-1-ol. To a stirred solution of (S)-1-(6′-amino-6-chloro-[2,3′-bipyridin]-5-yl)ethan-1-ol (500 mg, 2 mmol) and 3-methyl-4-(4,4,5,5-tetramethyl-1,3,2-dioxaborolan-2-yl)-1-(2,2,2-trifluoroethyl)-1H-pyrazole (697 mg, 2.4 mmol), K₂CO₃ (830 mg, 6 mmol) in dioxane (8 mL) and H₂O (1.5 mL) was added Pd(PPh₃)₄(231 mg, 0.2 mmol) at RT under N₂ atmosphere. The resulting mixture was stirred at 100° C. for 1 h under N₂ atmosphere. The precipitated solids were collected by filtration and washed with MeOH (3×20 mL). The resulting mixture was concentrated under reduced pressure. The residue was purified by Prep-TLC (CH₂Cl₂/MeOH 10:1) to afford the title compound. LCMS: 378 [M+H]⁺.

Step 2. Synthesis of Phenyl (S)-(5-(1-hydroxyethyl)-6-(3-methyl-1-(2,2,2-trifluoroethyl)-1H-pyrazol-4-yl)-[2,3′-bipyridin]-6′-yl) carbamate. To a stirred mixture of (S)-1-(6′-amino-6-(3-methyl-1-(2,2,2-trifluoroethyl)-1H-pyrazol-4-yl)-[2,3′-bipyridin]-5-yl) ethan-1-ol (186 mg, 493 mol) in THF (6 mL) was added 2,4,6-trimethylpyridine (84 mg, 690 mol) and phenyl chloroformate (54 mg, 345 mol) at 0° C. under N₂ atmosphere. The resulting mixture was stirred at RT for 2 h under N₂ atmosphere. The solvent was evaporated under reduced pressure. The residue was purified by Prep-TLC (CH₂Cl₂/MeOH 10:1) to afford the title compound. LCMS: 498 [M+H]⁺.

Step 3. Synthesis of 1-(5-((S)-1-hydroxyethyl)-6-(3-methyl-1-(2,2,2-trifluoroethyl)-1H-pyrazol-4-yl)-[2,3′-bipyridin]-6′-yl)-3-(1-(5-methylpyridin-2-yl) ethyl) urea. To a stirred solution of phenyl (S)-(5-(1-hydroxyethyl)-6-(3-methyl-1-(2,2,2-trifluoroethyl)-1H-pyrazol-4-yl)-[2,3′-bipyridin]-6′-yl) carbamate (169 mg, 340 mol) and TEA (103 mg, 1.02 mmol) in CH₂Cl₂ (7 mL) were added 1-(5-methylpyridin-2-yl) ethan-1-amine (69 mg, 510 mol) at RT under N₂ atmosphere. The resulting mixture was stirred at RT for 2 h under N₂ atmosphere. The resulting mixture was extracted with EtOAc (2×100 mL), washed with brine (3×50 mL) and dried over anhydrous Na₂SO₄. After filtration, the filtrate was concentrated under reduced pressure. The residue was purified by Prep-TLC (CH₂Cl₂/MeOH 20:1) to afford the title compound. LCMS: 540 [M+H]⁺.

Step 4. Synthesis of Separation of 1-(5-((S)-1-hydroxyethyl)-6-(3-methyl-1-(2,2,2-trifluoroethyl)-1H-pyrazol-4-yl)-[2,3′-bipyridin]-6′-yl)-3-((R*)-1-(5-methylpyridin-2-yl)ethyl)urea (Isomer 1) & 1-(5-((S)-1-hydroxyethyl)-6-(3-methyl-1-(2,2,2-trifluoroethyl)-1H-pyrazol-4-yl)-[2,3′-bipyridin]-6′-yl)-3-((S*)-1-(5-methylpyridin-2-yl)ethyl)urea (Isomer 2) 1-(5-((S)-1-hydroxyethyl)-6-(3-methyl-1-(2,2,2-trifluoroethyl)-1H-pyrazol-4-yl)-[2,3′-bipyridin]-6′-yl)-3-(1-(5-methylpyridin-2-yl) ethyl) urea (120 mg, 222 mol) was separated by Chiral-HPLC (Column: JW-CHIRAL ART Cellulose-SA, 20×250 mm, 5 m; Mobile Phase A: EtOH:DCM=1:1-HPLC, Mobile Phase B: Hex (0.5% 2M NH₃-MeOH)—HPLC; Flow rate: 20 mL/min; Gradient (B %): 70% B to 70% B in 17 min; Wave Length: 220/254 nm; RT1 (min): 8.377; RT2 (min): 15.362; Sample Solvent: EtOH:DCM=1:1-HPLC; Injection Volume: 1.0 mL; Number of Runs: 4). The first eluting isomer fraction was collected and concentrated under reduced pressure, the residue was re-dissolved in CH₃CN and H₂O and then lyophilized to afford isomer 1. The second eluting isomer fraction was collected and concentrated under vacuum, the residue was re-dissolved in CH₃CN and H₂O and then lyophilized to afford isomer 2.

Isomer 1:1-(5-((S)-1-hydroxyethyl)-6-(3-methyl-1-(2,2,2-trifluoroethyl)-1H-pyrazol-4-yl)-[2,3′-bipyridin]-6′-yl)-3-((R*)-1-(5-methylpyridin-2-yl)ethyl)urea (Isomer 1) ¹H NMR (400 MHz, DMSO-d6) δ (ppm): 9.45 (1H), 8.93 (1H), 8.64 (1H), 8.41 (1H), 8.37 (1H), 8.04 (2H), 7.92 (1H), 7.59 (1H), 7.54 (1H), 7.30 (1H), 5.28 (1H), 5.21-5.08 (2H), 5.01-4.91 (1H), 4.86 (1H), 2.30 (6H), 1.43 (3H), 1.34 (3H). LCMS: 540 [M+H]⁺. Chiral HPLC: Rt=1.949 min (Column: CHIRALPAK IA-3; Mobile Phase A: Hex (0.1% DEA): (EtOH:DCM=1:1)=70:30; Flow rate: 1 mL/min.)

Isomer 2: 1-(5-((S)-1-hydroxyethyl)-6-(3-methyl-1-(2,2,2-trifluoroethyl)-1H-pyrazol-4-yl)-[2,3′-bipyridin]-6′-yl)-3-((S*)-1-(5-methylpyridin-2-yl)ethyl)urea (Isomer 2)LCMS (Method H) Rt=1.293 mins (99.3% purity) ¹H NMR (400 MHz, DMSO-d6) δ (ppm): 9.45 (1H), 8.93 (1H), 8.65 (1H), 8.41 (1H), 8.37 (1H), 8.07-8.00 (2H), 7.93 (1H), 7.59 (1H), 7.54 (1H), 7.30 (1H), 5.28 (1H), 5.20-5.09 (2H), 5.02-4.91 (1H), 4.91-4.81 (1H), 2.30 (6H), 1.43 (3H), 1.34 (3H). LCMS: 540 [M+H]⁺. Chiral HPLC: Rt=2.718 min (Column: CHIRALPAK IA-3; Mobile Phase A: Hex (0.1% DEA): (EtOH:DCM=1:1)=70:30; Flow rate: 1 mL/min.) Example 36. Synthesis of (1S,2R)—N-(5-((S)-1-hydroxyethyl)-6-(1-(2,2,2-trifluoroethyl)-1,4,6,7-tetrahydropyrano[4,3-c]pyrazol-3-yl)-[2,3′-bipyridin]-6′-yl)-2-(5-methylpyrimidin-2-yl)cyclopropane-1-carboxamide (I-169)

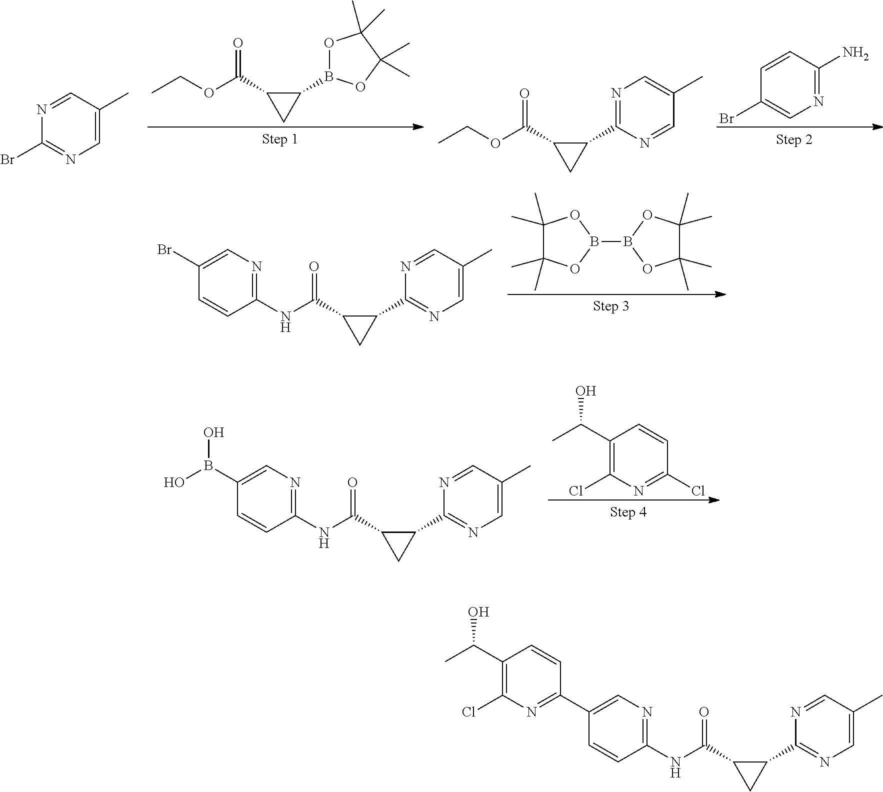

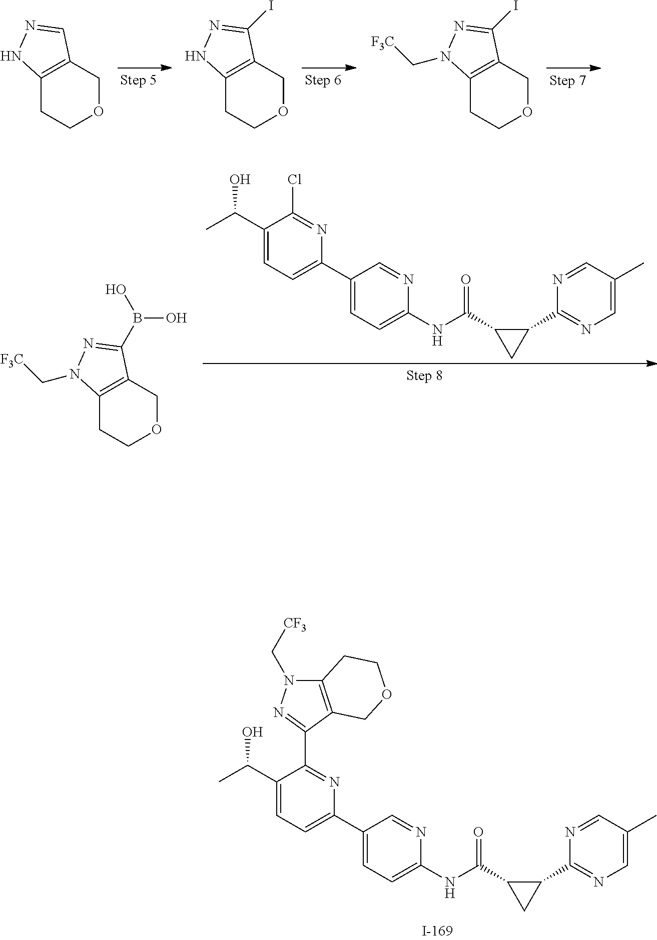

Step 1. Synthesis of ethyl (1S,2R)-2-(5-methylpyrimidin-2-yl)cyclopropane-1-carboxylate. To a solution of 2-bromo-5-methyl-pyrimidine (7.50 g, 43.35 mmol, 1 eq) and ethyl (1S,2R)-2-(4,4,5,5-tetramethyl-1,3,2-dioxaborolan-2-yl)cyclopropanecarboxylate (10.41 g, 43.35 mmol, 1 eq) in dioxane (100 mL) and H₂O (10 ml) was added bis(1-adamantyl)-butyl-phosphane (3.11 g, 8.67 mmol, 0.2 eq) Cs₂CO₃ (28.25 g, 86.70 mmol, 2 eq) and Pd(OAc)₂ (779 mg, 3.47 mmol, 0.08 eq). The mixture was stirred at 80° C. for 2 h. The reaction mixture was filtered and concentrated under reduced pressure to give a residue. The residue was purified by flash silica gel chromatography (Eluent of PE/EtOAc) to afford the title compound. ¹H NMR (400 MHz, CDCl₃) δ (ppm): 8.48 (s, 2H), 4.01-3.88 (m, 2H), 2.79 (d, 1H), 2.27 (s, 3H), 2.21-2.17 (m, 1H), 2.00-1.95 (m, 1H), 1.48-1.42 (m, 1H), 1.10 (t, 3H). LCMS: 207.3 [M+H]⁺.

Step 2. Synthesis of (1S,2R)—N-(5-bromopyridin-2-yl)-2-(5-methylpyrimidin-2-yl)cyclopropane-1-carboxamide. To a solution of ethyl (1S,2R)-2-(5-methylpyrimidin-2-yl)cyclopropanecarboxylate (1.80 g, 8.73 mmol, 1 eq) and 5-bromopyridin-2-amine (1.51 g, 8.73 mmol, 1 eq) in toluene (25 mL) was added LiHMDS (1 M in THF, 17.46 mL, 2 eq) at 0° C. under N₂ atmosphere. The mixture was warmed to 40° C. and stirred for 1 h under N₂ atmosphere. After cooling down to RT the reaction mixture was quenched with saturated aqueous NH₄Cl (20 mL) and extracted with EtOAc (25 mL×2). The combined organic phase was washed with brine (10 mL), dried with anhydrous Na₂SO₄, filtered and concentrated under reduced pressure to give a residue. The residue was purified by flash silica gel chromatography (Eluent of DCM/MeOH) to afford the title compound. ¹H NMR (400 MHz, DMSO-d₆) δ (ppm): 10.88 (s, 1H), 8.48 (s, 2H), 8.38 (d, 1H), 7.91-7.82 (m, 1H), 7.75 (d, 1H), 2.75 (d, 1H), 2.48-2.40 (m, 1H), 2.20 (s, 3H), 1.82 (d, 1H), 1.43-1.34 (m, 1H). LCMS: 332.9 [M+H]⁺.

Step 3. Synthesis of (6-((1S,2R)-2-(5-methylpyrimidin-2-yl) cyclopropane-1-carboxamido) pyridin-3-yl) boronic acid. A mixture of (1S,2R)—N-(5-bromopyridin-2-yl)-2-(5-methylpyrimidin-2-yl)cyclopropane-1-carboxamide (2.39 g, 7.17 mmol, 1 eq), 4,4,4′,4′,5,5,5′,5′-octamethyl-2,2′-bi(1,3,2-dioxaborolane) (2.73 g, 10.76 mmol, 1.5 eq), Pd(dppf)Cl₂·DCM (0.58 g, 0.72 mmol, 0.1 eq) and KOAc (2.11 g, 21.52 mmol, 3 eq) in dioxane (30 mL) was degassed and purged with N₂ for 3 times, and then the mixture was stirred at 80° C. for 2 h under N₂ atmosphere. After cooling down to RT, the reaction mixture was filtered through diatomite and the filtrate was concentrated under reduced pressure to give a crude product. The crude product was purified by reverse phase preparative HPLC (H₂O (0.1% FA)-ACN) to afford the title compound. ¹H NMR (400 MHz, DMSO-d₆) δ (ppm): 10.71 (s, 1H), 8.58 (d, 1H), 8.47 (s, 2H), 8.14 (s, 2H), 7.98-7.91 (m, 1H), 7.71 (d, 1H), 2.77-2.68 (m, 1H), 2.48-2.42 (m, 1H), 2.19 (s, 3H), 1.85-1.76 (m, 1H), 1.41-1.32 (m, 1H). LCMS: 299.0 [M+H]⁺.

Step 4. Synthesis of (1S,2R)—N-[5-[6-chloro-5-[(1S)-1-hydroxyethyl]-2-pyridyl]-2-pyridyl]-2-(5-methylpyrimidin-2-yl) cyclopropanecarboxamide. A mixture of (1S)-1-(2,6-dichloro-3-pyridyl) ethanol (500 mg, 2 mmol, 1.2 eq), Pd(dppf)Cl₂·DCM (177 mg, 216 mol, 0.1 eq), K₃PO₄ (1.15 g, 5 mmol, 2.5 eq) and [6-[[(1S,2R)-2-(5-methylpyrimidin-2-yl)cyclopropanecarbonyl]amino]-3-pyridyl]boronic acid (646 mg, 2 mmol, 1 eq) in dioxane (5 mL) and H₂O (1 mL) was degassed and purged with N₂ for 3 times. The reaction mixture was stirred at 40° C. for 2 h under N₂ atmosphere. The reaction mixture was filtered and the filtrate was concentrated under reduced pressure. The residue was purified by flash silica gel chromatography (Eluent of DCM/MeOH) to afford the title compound. ¹H NMR (400 MHz, DMSO-d₆) δ (ppm): 10.95 (s, 1H), 8.94 (d, 1H), 8.49 (s, 2H), 8.32-8.25 (m, 1H), 8.07-8.00 (m, 2H), 7.89 (d, 1H), 5.56 (d, 1H), 5.05-4.90 (m, 1H), 2.80-2.70 (m, 1H), 2.49-2.39 (m, 1H), 2.20 (s, 4H), 1.88-1.73 (m, 1H), 1.42-1.36 (m, 1H), 1.39-1.29 (m, 4H). LCMS: 410.3 [M+H]⁺.

Step 5. Synthesis of 3-iodo-1,4,6,7-tetrahydropyrano[4,3-c]pyrazole. To a stirred solution of 1,4,6,7-tetrahydropyrano[4,3-c]pyrazole (1 g, 8.05 mmol) in DMF (20 mL) were added KOH (903 mg, 16.11 mmol) at RT under N₂ atmosphere. The resulting mixture was stirred at RT for 10 min. Iodine (3.1 g, 12.0 mmol) was added to the mixture. The resulting mixture was stirred at RT overnight. The resulting mixture was extracted with EtOAc (3×80 mL). The combined organic layers were washed with water (2×40 mL), dried over anhydrous Na₂SO₄. After filtration, the filtrate was concentrated under reduced pressure. The residue was purified by Prep-TLC (CH₂Cl₂/MeOH 10:1) gradient to provide the title compound. LCMS: 251 [M+H]⁺.

Step 6. Synthesis of 3-iodo-1-(2,2,2-trifluoroethyl)-1,4,6,7-tetrahydropyrano[4,3-c]pyrazole. To a solution of 3-iodo-1,4,6,7-tetrahydropyrano[4,3-c]pyrazole (580 mg, 2.13 mmol) and 2,2,2-trifluoroethyl trifluoromethanesulfonate (1.2 g, 5.33 mmol) in MeCN (8 mL) were added Cs₂CO₃ (2.1 g, 5.33 mmol) at RT. The resulting mixture was stirred at RT overnight. The mixture was allowed to cool down to RT and concentrated under reduced pressure. The residue was purified via reverse phase flash chromatography (C18 silica gel) using a 30% to 50% ACN in water (with 0.1% FA) gradient to provide the title compound. LCMS: 333 [M+H]⁺.

Step 7. Synthesis of (1-(2,2,2-trifluoroethyl)-1,4,6,7-tetrahydropyrano[4,3-c]pyrazol-3-yl)boronic acid. To a solution of 3-iodo-1-(2,2,2-trifluoroethyl)-1,4,6,7-tetrahydropyrano[4,3-c]pyrazole (650 mg, 1.95 mmol) in THF (4 mL) was added i-PrMgBr (2.5 mL, 2.93 mmol, 1.3 M in THF) dropwise at −78° C. under N₂ atmosphere. The resulting mixture was stirred at −78° C. for 30 min under N₂ atmosphere. To the above mixture was added trimethyl borate (813 mg, 7.82 mmol) dropwise at −60° C. The resulting mixture was stirred at RT for additional 2 h. The reaction was poured into sat. NH₄Cl (aq.) at 0° C. and extracted with EtOAc (3×50 mL). The residue was directly purified via reverse phase flash chromatography (C18 silica gel) using 40% to 50% ACN in water (with 0.1% FA) gradient to provide the title compound. LCMS: 251 [M+H]⁺.

Step 8. Synthesis of (1S,2R)—N-(5-((S)-1-hydroxyethyl)-6-(1-(2,2,2-trifluoroethyl)-1,4,6,7-tetrahydropyrano[4,3-c]pyrazol-3-yl)-[2,3′-bipyridin]-6′-yl)-2-(5-methylpyrimidin-2-yl)cyclopropane-1-carboxamide. To a stirred solution of (1S,2R)—N-(6-chloro-5-((S)-1-hydroxyethyl)-[2,3′-bipyridin]-6′-yl)-2-(5-methylpyrimidin-2-yl)cyclopropane-1-carboxamide (100 mg, 0.244 mmol) and (1-(2,2,2-trifluoroethyl)-1,4,6,7-tetrahydropyrano[4,3-c]pyrazol-3-yl)boronic acid (243 mg, 0.98 mmol) in 1.4-dioxane (3.2 mL), H₂O (0.4 mL) were added Pd(PPh₃)₄ (56.3 mg, 0.05 mmol) and K₂CO₃ (101 mg, 0.73 mmol) at RT under N₂ atmosphere. The resulting mixture was stirred at 80° C. for 2 h under N₂ atmosphere. The mixture was allowed to cool down to RT. The reaction was extracted with EtOAc (3×10 mL), washed with brine (3×10 mL), dried over anhydrous Na₂SO₄ and concentrated under reduced pressure. The crude was purified by Prep TLC (CH₂Cl₂/MeOH 10:1) to afford the crude product. The crude product was purified by Prep HPLC (Column: X-Select Prep OBD C18 Column, 30×150 mm, 5 m; Mobile Phase A: water (10 mmol/L NH₄HCO₃), Mobile Phase B: ACN; Flow rate: 60 mL/min; Gradient (B %): 37% B to 47% B in 10 min; Wave Length: 254 nm/220 nm; RT1(min): 8.25) to afford the product (ee value <95%). The mixture was further purified by Prep-Chiral-HPLC with the following conditions (Column: JW—CHIRAL ART Cellulose-SB, 20×250 mm, 5 μm; Mobile Phase A: EtOH:DCM=1:1-HPLC, Mobile Phase B: Hex (0.1% 2M NH₃-MeOH)—HPLC; Flow rate: 20 mL/min; Gradient (B %): 50% B to 50% B in 7 min % B; Wave Length: 220/254 nm; RT1(min): 3.771; RT2(min): 5.725; Sample Solvent: EtOH:DCM=1:1-HPLC; Injection Volume: 1.0 mL; Number Of Runs: 2) to provide the title compound. LCMS: 580 [M+H]⁺. ¹H NMR (400 MHz, DMSO-d6) δ (ppm): 10.87 (1H), 8.99-8.94 (1H), 8.50 (2H), 8.36-8.28 (1H), 8.16 (1H), 7.98-7.88 (2H), 5.81-5.70 (1H), 5.25 (1H), 5.22-5.11 (2H), 4.96 (1H), 4.86 (1H), 3.99-3.84 (2H), 2.85-2.72 (3H), 2.54 (1H), 2.21 (3H), 1.90-1.81 (1H), 1.45-1.34 (4H).

### Example 37. Synthesis of (1S,2R)-2-(1,6-dimethyl-2-oxo-1,2-dihydropyridin-3-yl)-N-(5-((S)-1-hydroxyethyl)-6-(3-methyl-1-(2,2,2-trifluoroethyl)-1H-pyrazol-4-yl)-[2,3′-bipyridin]-6′-yl) cyclopropane-1-carboxamide (I-170)

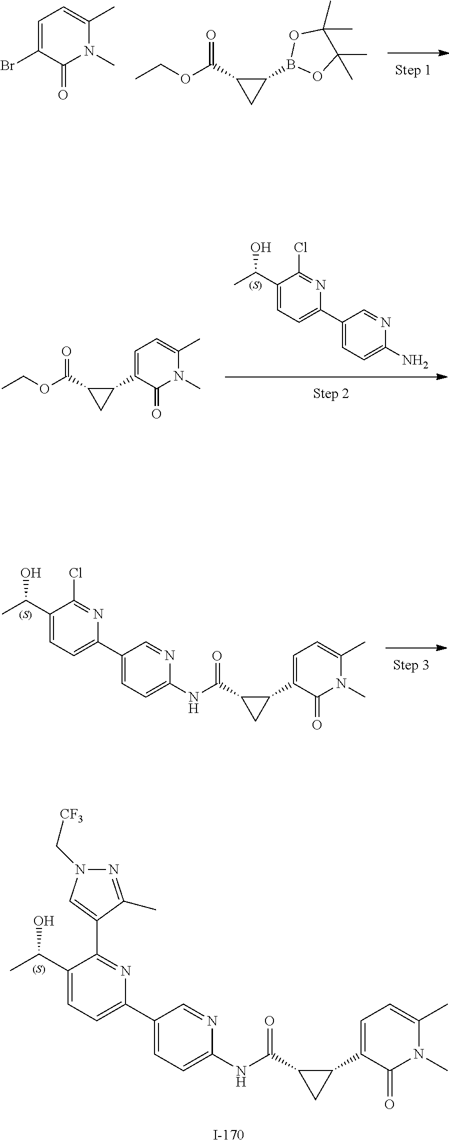

Step 1. Synthesis of ethyl (1S,2R)-2-(1,6-dimethyl-2-oxo-1,2-dihydropyridin-3-yl) cyclopropane-1-carboxylate. A mixture of 3-bromo-1,6-dimethylpyridin-2(1H)-one (500 mg, 2.47 mmol), ethyl (1S,2R)-2-(4,4,5,5-tetramethyl-1,3,2-dioxaborolan-2-yl) cyclopropane-1-carboxylate (1.20 g, 4.95 mmol), Pd(OAc)₂ (56 mg, 0.25 mmol), di(1-adamantyl)-N-butylphosphine (177 mg, 0.50 mmol) and Cs₂CO₃ (2.42 g, 7.42 mmol) in Dioxane (10 mL)/H₂O (1 mL) was stirred at 100° C. for 1 h under N₂ atmosphere. The mixture was allowed to cool down to RT. The resulting mixture was concentrated under reduced pressure. The residue was purified via reverse flash chromatography (C18 silica gel) using a 10% to 70% ACN in water (with 10 mmol/L NH₄HCO₃) gradient to provide the title compound. LCMS: 236 [M+H]⁺.

Step 2. Synthesis of (1S,2R)—N-(6-chloro-5-((S)-1-hydroxyethyl)-[2,3′-bipyridin]-6′-yl)-2-(1,6-dimethyl-2-oxo-1,2-dihydropyridin-3-yl) cyclopropane-1-carboxamide. To a stirred solution of ethyl (1S,2R)-2-(1,6-dimethyl-2-oxo-1,2-dihydropyridin-3-yl) cyclopropane-1-carboxylate (200 mg, 0.850 mmol) and (S)-1-(6′-amino-6-chloro-[2,3′-bipyridin]-5-yl) ethan-1-ol (106 mg, 0.43 mmol) in THF (10 mL) was added trimethylaluminium (2.0M in n-hexane) (0.43 mL, 0.85 mmol) dropwise at 0° C. under N₂ atmosphere. The resulting mixture was stirred at 80° C. for 2 h under N₂ atmosphere. The mixture was allowed to cool down to RT. The resulting mixture was concentrated under reduced pressure. The residue was purified via reverse flash chromatography (C18 silica gel) using a 10% to 50% ACN in water (with 10 mmol/L NH₄HCO₃) gradient to provide the title compound. LCMS: 439 [M+H]⁺.

Step 3. Synthesis of (1S,2R)-2-(1,6-dimethyl-2-oxo-1,2-dihydropyridin-3-yl)-N-(5-((S)-1-hydroxyethyl)-6-(3-methyl-1-(2,2,2-trifluoroethyl)-1H-pyrazol-4-yl)-[2,3′-bipyridin]-6′-yl) cyclopropane-1-carboxamide. A mixture of (1S,2R)—N-(6-chloro-5-((S)-1-hydroxyethyl)-[2,3′-bipyridin]-6′-yl)-2-(1,6-dimethyl-2-oxo-1,2-dihydropyridin-3-yl) cyclopropane-1-carboxamide (60 mg, 0.137 mmol), 3-methyl-4-(4,4,5,5-tetramethyl-1,3,2-dioxaborolan-2-yl)-1-(2,2,2-trifluoroethyl)-1H-pyrazole (198 mg, 0.69 mmol), Pd(dppf)Cl₂·CH₂Cl₂ (15.8 mg, 0.014 mmol) and Cs₂CO₃ (56.6 mg, 0.41 mmol) in dioxane (5 mL)/H₂O (0.5 mL) was stirred at 100° C. for 2 h under N₂ atmosphere. The mixture was allowed to cool down to RT. The resulting mixture was concentrated under reduced pressure. The residue was purified by Prep-TLC (CH₂Cl₂/MeOH 10:1) to afford the crude product, which was further purified by chiral-HPLC (Column: JW-CHIRAL ART Cellulose-SA, 20×250 mm, Sum; Mobile Phase A: IPA:DCM=1:1-HPLC, Mobile Phase B: Hex (0.5% 2M NH₃-MeOH)—HPLC; Flow rate: 20 mL/min; Gradient (B %): 40% B to 40% B in 11 min % B; Wave Length: 220/254 nm; RT1(min): 5.465; RT2(min): 6.462; Sample Solvent: EtOH:DCM=1:1-HPLC; Injection Volume: 1.0 mL; Number of Runs: 8) to provide the title compound. LCMS: 567.15 [M+H]⁺. ¹H NMR (400 MHz, DMSO-d6) δ (ppm): 10.76 (1H), 8.98 (1H), 8.35-8.33 (1H), 8.05-8.02 (2H), 7.97 (1H), 7.92 (1H), 7.15 (1H), 6.07 (1H), 5.28 (1H), 5.24-5.10 (2H), 4.85 (1H), 3.37 (3H), 2.46-2.35 (2H), 2.33-2.28 (6H), 1.49-1.45 (1H), 1.32 (3H), 1.29-1.24 (1H).

### Example 38. Synthesis of (1S,2R)-2-(1,6-dimethyl-2-oxo-1,2-dihydropyridin-3-yl)-N-(5-((S)-1-hydroxyethyl)-6-(3-methyl-1-(2,2,2-trifluoroethyl)-1H-pyrazol-4-yl)-[2,3′-bipyridin]-6′-yl) cyclopropane-1-carboxamide (I-171)

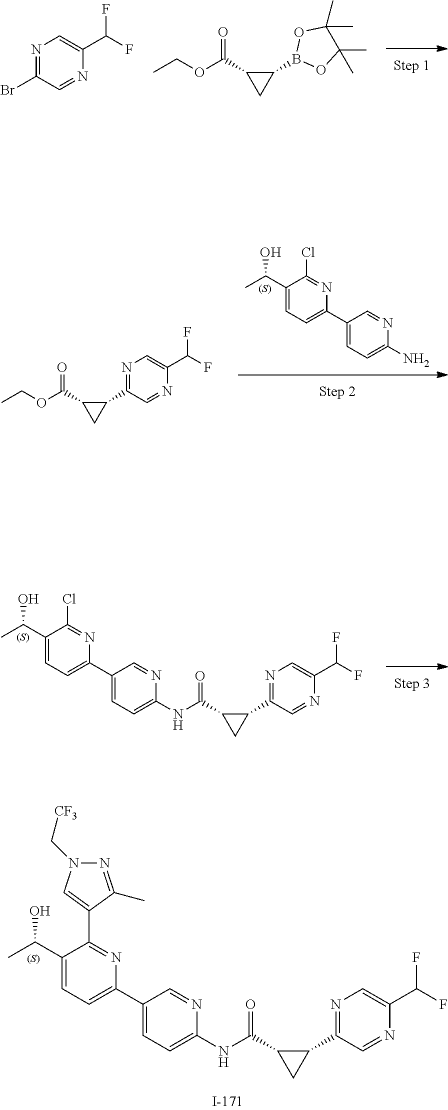

Step 1. Synthesis of ethyl (1S,2R)-2-(5-(difluoromethyl)pyrazin-2-yl)cyclopropane-1-carboxylate. To the solution of 2-bromo-5-(difluoromethyl)pyrazine (360 mg, 1.72 mmol) and ethyl (1S,2R)-2-(4,4,5,5-tetramethyl-1,3,2-dioxaborolan-2-yl)cyclopropane-1-carboxylate (1.03 g, 4.31 mmol) in toluene (5 mL) were added Cs₂CO₃ (1.68 g, 5.17 mmol), Pd(OAc)₂ (38.7 mg, 172 mol) and cataCXium A (124 mg, 345 mol) at RT. The mixture was stirred at 100° C. for 1.0 h under N₂ atmosphere. The reaction mixture was cooled to RT, filtered and concentrated under reduced pressure to give a residue. The residue was purified by Prep-TLC with petroleum ether/ethyl acetate (2/1) to afford the title product. LCMS: 243 [M+H]⁺.

Step 2. Synthesis of (1S,2R)—N-(6-chloro-5-((S)-1-hydroxyethyl)-[2,3′-bipyridin]-6′-yl)-2-(5-(difluoromethyl)pyrazin-2-yl)cyclopropane-1-carboxamide. A mixture of (S)-1-(6′-amino-6-chloro-[2,3′-bipyridin]-5-yl)ethan-1-ol (100 mg, 400 mol) and ethyl (1S,2R)-2-(5-(difluoromethyl)pyrazin-2-yl)cyclopropane-1-carboxylate (214 mg, 880 mol) in THF (4 mL) was degassed and purged with N₂ for 3 times and cooled to 0° C., and then LiHMDS (1.0 M in THF, 1.20 mL, 1.20 mmol) was added to the mixture at 0° C. under N₂ atmosphere. The mixture was stirred at RT for 3.0 h under N₂ atmosphere. The reaction mixture was quenched by saturated NH₄Cl aqueous solution (40 mL), then extracted with ethyl acetate (50 mL×3). The combined organic layers were washed with brine, dried over Na₂SO₄, filtered and concentrated under reduced pressure to give a residue. The residue was purified by Prep-TLC with C CH₂Cl₂/MeOH (8/1) to afford the title product. LCMS: 446 [M+H]⁺.

Step 3. Synthesis of (1S,2R)-2-(5-(difluoromethyl)pyrazin-2-yl)-N-(5-((S)-1-hydroxyethyl)-6-(3-methyl-1-(2,2,2-trifluoroethyl)-1H-pyrazol-4-yl)-[2,3′-bipyridin]-6′-yl)cyclopropane-1-carboxamide. To a solution of (1S,2R)—N-(6-chloro-5-((S)-1-hydroxyethyl)-[2,3′-bipyridin]-6′-yl)-2-(5-(difluoromethyl)pyrazin-2-yl)cyclopropane-1-carboxamide (70 mg, 157 mol) and 3-methyl-4-(4,4,5,5-tetramethyl-1,3,2-dioxaborolan-2-yl)-1-(2,2,2-trifluoroethyl)-1H-pyrazole (319 mg, 1.10 mmol) in dioxane (2 mL) and H₂O (0.2 mL) were added K₃PO₄ (100 mg, 471 mol) and Pd(dppf)Cl₂·CH₂Cl₂ (12.8 mg, 16.0 mol) at RT. The mixture was stirred at 80° C. for 1.0 h under N₂ atmosphere and cooled down to RT. The mixture was filtered and concentrated under reduced pressure. The residue was purified by Prep-HPLC (Column: X-Select Prep OBD C18 Column, 30×150 mm, 5 m; Mobile Phase A: Water (0.1% FA), Mobile Phase B: ACN; Flow rate: 60 mL/min; Gradient (B %): 41% B to 51% B in 10 min; Wave Length: 254 nm/220 nm; RT1(min): 7.83). The crude product was further purified by Perp-Chiral HPLC (Column: JW-CHIRALPAK IM, 20×250 mm, 5 m; Mobile Phase A: EtOH:DCM=1:1, Mobile Phase B: Hex (0.5% 2M NH₃-MeOH); Flow rate: 20 mL/min; Gradient (B %): 60% B to 60% B in 8 min; Wave Length: 220/254 nm; RT1(min): 5.512; RT2(min): 7.536) to afford the title compound. LCMS: 574 [M+H]⁺. ¹H NMR (400 MHz, DMSO-d6) δ (ppm): 11.05 (1H), 8.99 (1H), 8.78 (1H), 8.74 (1H), 8.35-8.31 (1H), 8.05-8.02 (2H), 7.92 (1H), 7.86 (1H), 7.08 (1H), 5.28 (1H), 5.16-5.09 (2H), 4.89-4.81 (1H), 2.85-2.80 (1H), 2.69-2.61 (1H), 2.28 (3H), 1.93-1.87 (1H), 1.58-1.52 (1H), 1.32 (3H).

### Example 39. Synthesis of N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 1) & N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 2) (I-172 & I-173)

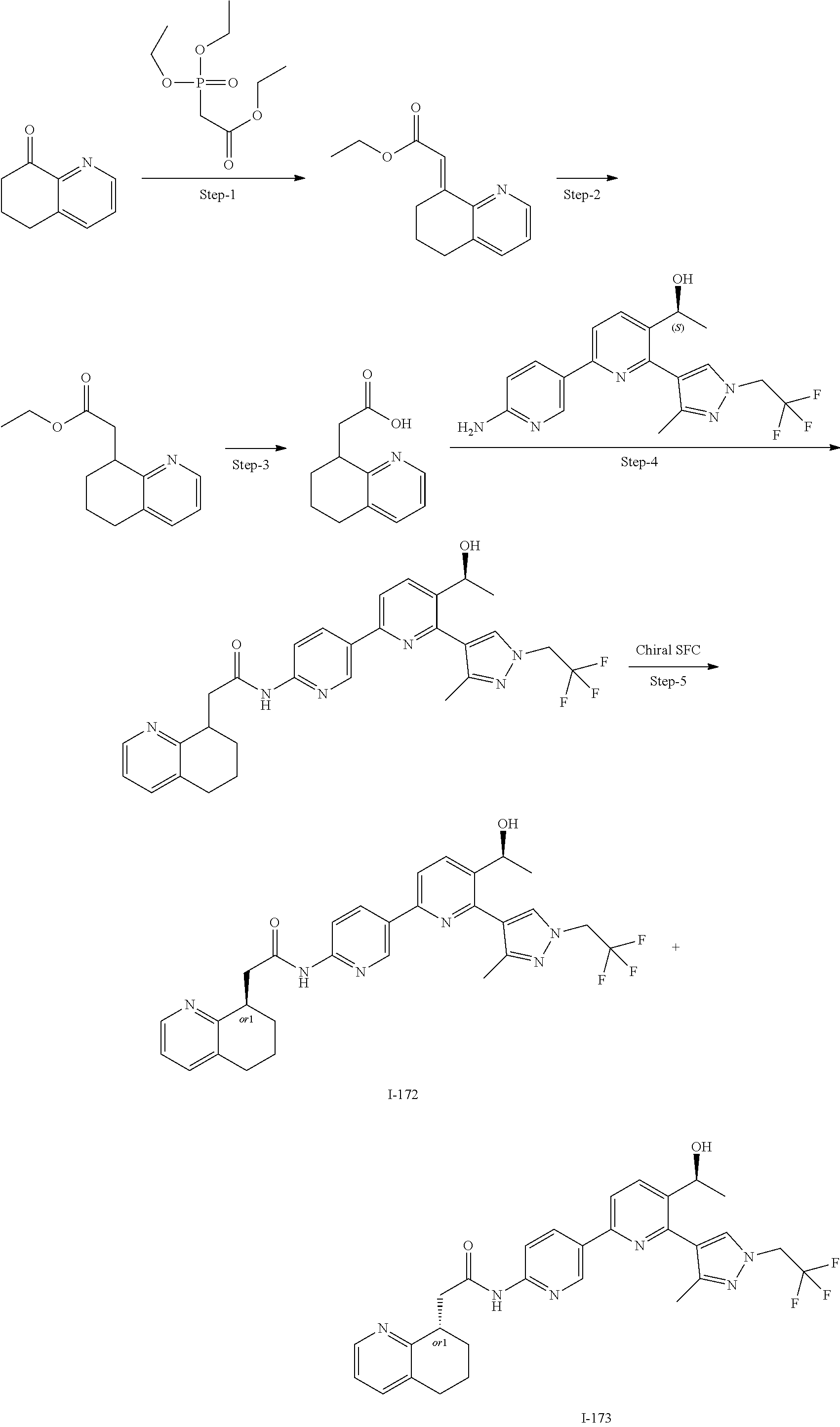

Step 1: Synthesis of ethyl (E)-2-(6,7-dihydroquinolin-8(5H)-xylidine) acetate. To a stirred suspension of sodium hydride, 57-63% oil dispersion (0.5 g, 20.38 mmol, 1.5 equiv.) in anhydrous tetrahydrofuran (20 mL) was added a solution of ethyl 2-(diethoxy phosphoryl) acetate (6 g, 27.17 mmol, 2 equiv.) at 0° C. and the resulting reaction mixture was stirred at RT for 30 min. 6,7-Dihydroquinolin-8(5H)-one (2 g, 13.58 mmol, 1 equiv.) in anhydrous tetrahydrofuran (20 mL) was added at RT and the resulting reaction mixture was stirred at 50° C. for 2 h. The reaction mixture quenched with saturated aqueous ammonium chloride and extracted with diethyl ether (2×150 ml). The combined organic layer was washed with brine and dried over anhydrous sodium sulphate, filtered and concentrated under reduced pressure to afford crude compound. The crude product was purified by silica gel chromatography (Eluent: 0-20% EtOAc in petroleum ether) to afford the title compound. LC-MS: 218.07 [M+H]⁺.

Step 2: Synthesis of ethyl 2-(5,6,7,8-tetrahydroquinolin-8-yl) acetate. To a stirred solution of ethyl (E)-2-(6,7-dihydroquinolin-8(5H)-xylidine) acetate (1.8 g, 8.28 mmol, 1 equiv.) in methanol (30 mL) was added 10% palladium on activated carbon (0.9 g, 0.82 mmol, 0.1 equiv.) and the resulting reaction mixture was stirred at room temperature under hydrogen atmosphere (~15 psi) for 16 h. After completion, the reaction mixture was filtered through the celite pad and washed with methanol (100 mL) and the combined filtrate was concentrated under reduced pressure to afford the title compound. ¹H NMR (400 MHz, CDCl₃) δ (ppm): 8.36 (d, J=3.7 Hz, 1H), 7.35 (d, J=7.6 Hz, 1H), 7.03-7.00 (m, 1H), 4.20-4.13 (m, 2H), 3.38 (brs, 1H), 3.15-3.10 (m, 1H), 2.79-2.75 (m, 2H), 2.54 (t, J=9.2 Hz, 1H), 2.09-2.08 (m, 1H), 1.88-1.78 (m, 1H), 1.77-1.70 (m, 2H), 1.26 (t, J=7.2 Hz, 3H). LC-MS: 220.08 [M+H]⁺.

Step 3: Synthesis of 2-(5,6,7,8-tetrahydroquinolin-8-yl) acetic acid. To a stirred solution of ethyl 2-(5,6,7,8-tetrahydroquinolin-8-yl) acetate (1.5 g, 6.84 mmol, 1 equiv.) in tetrahydrofuran (10 mL) was added lithium hydroxide monohydrate (0.5 g, 13.68 mmol, 2 equiv) in water (2 mL) and the resulting reaction mixture was stirred at RT for 16 h. The resulting mixture was concentrated under reduced pressure. The residue was diluted with water, acidified (pH=2) with saturated aqueous citric acid solution and extracted with ethyl acetate (2×100 mL). The combined organic layers were washed with brine, dried over anhydrous sodium sulphate, filtered and concentrated under reduced pressure to afford the title compound. ¹H NMR (400 MHz, DMSO-d₆): δ (ppm): 12.10 (s, 1H), 8.33-8.31 (m, 1H), 7.47-7.45 (m, 1H), 7.14-7.11 (m, 1H), 3.18-3.15 (m, 1H), 2.99 (d, J=4.4 Hz, 1H), 2.94-2.72 (m, 2H), 2.39-2.32 (m, 1H), 2.02-1.99 (m, 1H), 1.83-1.80 (m, 1H), 1.79-1.59 (m, 2H). LC-MS=192.07 [M+H]⁺.

Step 4. Synthesis of N-(5-((S)-1-hydroxyethyl)-6-(3-methyl-1-(2,2,2-trifluoroethyl)-1H-pyrazol-4-yl)-[2,3′-bipyridin]-6′-yl)-2-(5,6,7,8-tetrahydroquinolin-8-yl) acetamide. To a stirred solution of (S)-1-(6′-amino-6-(3-methyl-1-(2,2,2-trifluoroethyl)-1H-pyrazol-4-yl)-[2,3′-bipyridin]-5-yl) ethan-1-ol (200 mg, 0.53 mmol, 1 equiv.) in pyridine (5 mL) were added 1-Ethyl-3-(3-dimethylaminopropyl) carbodiimide hydrochloride (205 mg, 1.06 mmol, 2 equiv.) and 2-(5,6,7,8-tetrahydroquinolin-8-yl) acetic acid (152 mg, 0.75 mmol, 1.5 equiv.) at room temperature. The resulting reaction mixture was stirred at 100° C. for 16 h. The reaction mixture was concentrated underreduced pressure and the residue diluted with ice cold water. The obtained solid was filtered, washed with pentane, dried under vacuum to get crude compound. The crude compound was purified by the preparative HPLC (Mobile Phase A:—10 mM Ammonium Bicarbonate in Water Mobile Phase B: 100% Acetonitrile Diluent: Acetonitrile water Gradient program (Time/% Mobile Phase B)—0/40,2/40,12/85,14/100 Flow Rate: 15 mL/min INST ID: ANL-MCL3-apms-008 YMC Trait C18 (25×150 mm), 10u ANL-PREP-HW-011). Pure fractions were distilled and lyophilization to give the title compound. LCMS: 551.23 [M+H]⁺.

Step 5. Separation of N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 1) & N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 2). N-(5-((S)-1-hydroxyethyl)-6-(3-methyl-1-(2,2,2-trifluoroethyl)-1H-pyrazol-4-yl)-[2,3′-bipyridin]-6′-yl)-2-(5,6,7,8-tetrahydroquinolin-8-yl) acetamide was separated by preparative chiral SFC (Column/Dimensions: CHIRALPAK IK (250×30×5μ), % CO₂: 70%, % Co solvent: 30% (0.5% Methanolic ammonia in IPA: Methanol (9:1)), Total Flow: 100 mL/min, Back Pressure:100 Bar, Temperature: 30.0° Wavelength: 302 NM, Stack time: 13.8 mins/injection, Load ability: 7.5 mg/injection, No. Of Injections: 9) to give the title compounds as peak 1 (Isomer1) and peak 2 (Isomer 2).

Peak 1: N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 1) ¹H NMR (400 MHz, DMSO-d6) δ (ppm): 10.72 (1H), 9.02 (1H), 8.47 (1H), 8.34 (1H), 8.25 (1H), 8.07-8.03 (2H), 7.96 (1H), 7.47 (1H), 7.15-7.12 (1H), 5.29 (1H), 5.17 (2H), 4.88-4.85 (1H), 3.41 (1H), 3.17-3.12 (1H), 2.78-2.61 (3H), 2.30 (3H), 2.01-1.98 (1H), 1.88-1.84 (1H), 1.70-1.62 (2H), 1.34 (3H). LCMS: 551.38 [M+H]⁺. Chiral SFC: Rt=3.52 min (CHIRALPAK IK (4.6×150 mm) 5 m; co solvent: isopropanol; 3 m/min; % of CO₂: 70; % of co solvent: 30; 1500 psi; 30° C.).

Peak 2: N-(5-((S)-1-hydroxyethyl)-6-(3-methyl-1-(2,2,2-trifluoroethyl)-1H-pyrazol-4-yl)-[2,3′-bipyridin]-6′-yl)-2-((R*)-5,6,7,8-tetrahydroquinolin-8-yl)acetamide (Isomer 2) ¹H NMR (400 MHz, DMSO-d6) δ (ppm): 10.71 (1H), 9.02 (1H), 8.47 (1H), 8.34 (1H), 8.25 (1H), 8.07-8.03 (2H), 7.96 (1H), 7.47 (1H), 7.15-7.12 (1H), 5.29 (1H), 5.17 (2H), 4.88-4.85 (1H), 3.41 (1H), 3.17-3.12 (1H), 2.78-2.61 (3H), 2.30 (3H), 2.01-1.98 (1H), 1.88-1.84 (1H), 1.70-1.62 (2H), 1.34 (3H). LCMS: 551.38 [M+H]⁺. Chiral SFC: Rt=4.67 min (CHIRALPAK IK (4.6×150 mm) 5 μm; co solvent: isopropanol; 3 m/min; % of CO₂: 70; % of co solvent: 30; 1500 psi; 30° C.).

### Example 40. Synthesis of (1S,2R)-2-(5-(difluoromethyl)pyridin-2-yl)-N-(5-((2R*,4R**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-(difluoromethyl)pyridin-2-yl)-N-(5-((2R*,4S**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 2) & (1S,2R)-2-(5-(difluoromethyl)pyridin-2-yl)-N-(5-((2R*,4S**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 3) & (1S,2R)-2-(5-(difluoromethyl)pyridin-2-yl)-N-(5-((2R*,4R**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 4) (I-174 Isomer 1& I-175 Isomer 2 & I-176 Isomer 3 & I-177 Isomer 4)

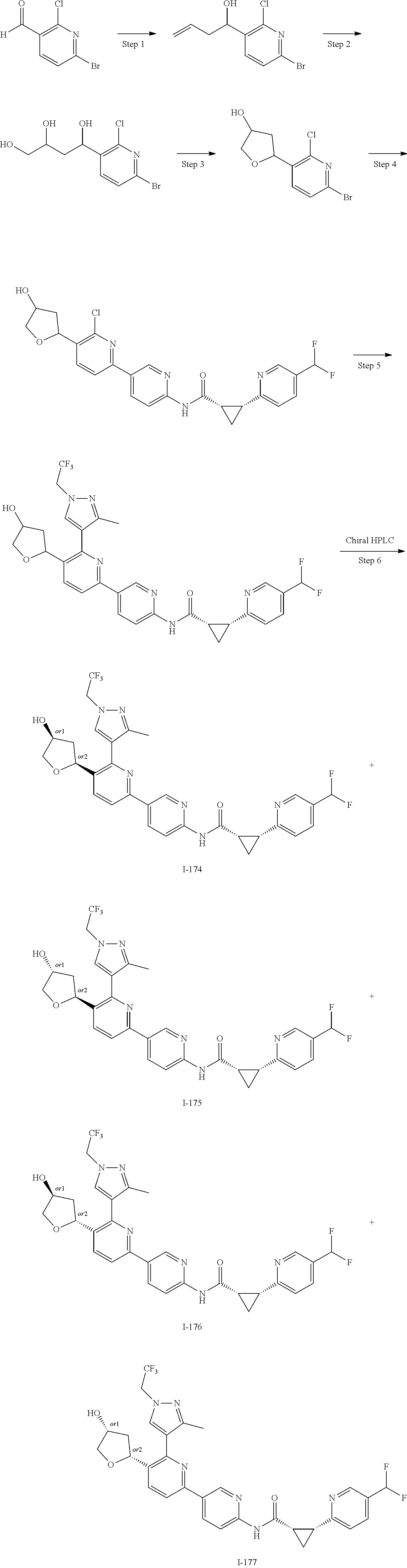

Step 1. Synthesis of 1-(6-bromo-2-chloropyridin-3-yl)but-3-en-1-ol. 6-bromo-2-chloronicotinaldehyde (1.0 g, 4.57 mmol) in CH₂Cl₂ (10 mL) were added 3-bromoprop-1-ene (910 mg, 5.44 mmol) at RT under N₂ atmosphere. The resulting mixture was stirred at 25° C. overnight under N₂ atmosphere. The resulting mixture was concentrated under reduced pressure. The residue was purified by Prep-TLC (CH₂Cl₂/MeOH 10:1) to afford the title compound. LCMS: 262 [M+H]⁺.

Step 2. Synthesis of 4-(6-bromo-2-chloropyridin-3-yl)butane-1,2,4-triol. To a stirred solution of 1-(6-bromo-2-chloropyridin-3-yl) but-3-en-1-ol (700 mg, 2.67 mmol) and K₂S₂O₈ (72 mg, 0.27 mmol), Citric Acid (666 mg, 3.47 mmol) in t-BuOH (6 mL) and H₂O (6 mL) was added 4-methylmorpholin-4-ium-4-olate hydrate (469 mg, 3.47 mmol) at RT under N₂ atmosphere. The resulting mixture was stirred at 40° C. for 2.0 h under N₂ atmosphere. The resulting mixture was concentrated under reduced pressure. The residue was purified via reverse phase flash chromatography (C18 silica gel) using a 10% to 30% ACN in water (with 0.1% NH₄HCO₃) gradient to provide the title compound. LCMS: 296 [M+H]⁺.

Step 3. Synthesis of 5-(6-bromo-2-chloropyridin-3-yl)tetrahydrofuran-3-ol. To a stirred solution of 4-(6-bromo-2-chloropyridin-3-yl) butane-1,2,4-triol (700 mg, 2.36 mmol) in 1,2-DCE (35 mL) was added TfOH (0.24 mL) dropwise at RT. The resulting mixture was stirred at 80° C. for 1.0 h. The residue was purified via reverse flash chromatography (C18 silica gel) using a 20% to 50% ACN in water (with 0.1% NH₄HCO₃) gradient to provide the title compound. LCMS: 278 [M+H]⁺.

Step 4. Synthesis of (1S,2R)—N-(6-chloro-5-(4-hydroxytetrahydrofuran-2-yl)-[2,3′-bipyridin]-6′-yl)-2-(5-(difluoromethyl)pyridin-2-yl)cyclopropane-1-carboxamide. To a stirred solution of 5-(6-bromo-2-chloropyridin-3-yl) tetrahydrofuran-3-ol (90 mg, 0.32 mmol) and (6-((1S,2R)-2-(5-(difluoromethyl)pyridin-2-yl)cyclopropane-1-carboxamido)pyridin-3-yl)boronic acid (129 mg, 0.39 mmol) in 1,4-dioxane (2 mL) and H₂O (0.2 mL) were added Pd(dtbpf)Cl₂ (21 mg, 0.032 mmol) at RT under N₂ atmosphere. The resulting mixture was stirred at 100° C. for 2.0 h under N₂ atmosphere. The mixture was allowed to cool down to RT and was concentrated under reduced pressure. The residue was purified by Prep-TLC (petroleum ether/EtOAc 3:1) to afford the title compound. LCMS: 487 [M+H]⁺.

Step 5. Synthesis of (1S,2R)-2-(5-(difluoromethyl)pyridin-2-yl)-N-(5-(4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide. To a stirred solution of (1S,2R)—N-(6-chloro-5-(4-hydroxytetrahydrofuran-2-yl)-[2,3′-bipyridin]-6′-yl)-2-(5-(difluoromethyl) pyridin-2-yl)cyclopropane-1-carboxamide (90 mg, 0.19 mmol) and 3-methyl-4-(4,4,5,5-tetramethyl-1,3,2-dioxaborolan-2-yl)-1-(2,2,2-trifluoroethyl)-1H-pyrazole (64 mg, 0.22 mmol) in 1,4-dioxane (1 mL) and H₂O (0.1 mL) was added K₂CO₃ (77 mg, 0.56 mmol) and Pd(dppf)Cl₂·CH₂Cl₂ (15 mg, 0.02 mmol) at RT under N₂ atmosphere. The resulting mixture was stirred at 80° C. for 1 h under N₂ atmosphere. The mixture was allowed to cool down to RT and was concentrated under reduced pressure. The residue was purified by reversed-phase flash chromatography with the following conditions: column, C18 silica gel; mobile phase, MeCN in Water (10 mmol/L NH₄HCO₃), 20% to 50% gradient in 30 min; detector, UV 254 nm to afford the title compound. LCMS: 615 [M+H]⁺.

Step 6. Separation of (1S,2R)-2-(5-(difluoromethyl)pyridin-2-yl)-N-(5-((2R*,4R**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 1) & (1S,2R)-2-(5-(difluoromethyl)pyridin-2-yl)-N-(5-((2R*,4S**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 2) & (1S,2R)-2-(5-(difluoromethyl)pyridin-2-yl)-N-(5-((2R*,4S**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 3) & (1S,2R)-2-(5-(difluoromethyl)pyridin-2-yl)-N-(5-((2R*,4R**)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 4). (1S,2R)-2-(5-(difluoromethyl)pyridin-2-yl)-N-(5-(4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide was purified by preparative chiral HPLC (Column: JW-CHIRALPAK IK, 20×250 mm, 5 μm; Mobile Phase A: EtOH:DCM=1:1-HPLC, Mobile Phase B: Hex (0.1% 2M NH₃-MeOH)—HPLC; Flow rate: 20 ml/min; Gradient (B %): 70% B to 70% B in 18 min; Wave Length: 220/254 nm; RT1(min): 6.72; RT2(min): 11.3; Sample Solvent: EtOH:DCM=1:1-HPLC; Injection Volume: 1.5 mL; Number Of Runs: 4) to afford the title compounds as peak 1 (Isomer 1) and peak 2 (Isomer 2) and peak 3 (Isomer 3) and peak 4 (Isomer 4).

Peak 1: (1S,2R)-2-(5-(difluoromethyl)pyridin-2-yl)-N-(5-((2R*,4R*)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 1) ¹H NMR (400 MHz, DMSO-d6) δ (ppm): 10.96 (1H), 9.01-8.97 (1H), 8.60 (1H), 8.37-8.31 (1H), 8.01 (1H), 7.96 (1H), 7.94-7.84 (3H), 7.47 (1H), 7.08 (1H), 5.18-5.04 (3H), 4.99 (1H), 4.44 (1H), 4.15-4.08 (1H), 3.68-3.60 (1H), 2.82-2.72 (1H), 2.62-2.55 (1H), 2.26 (3H), 2.12-2.04 (1H), 1.92-1.76 (2H), 1.51-1.42 (1H). LCMS: 615 [M+H]⁺. Chiral HPLC: Rt=2.062 min (CHIRALPAK IF-3; Mobile Phase A: Hex (0.1% DEA): (EtOH:DCM=1:1)=65: 35; Flow rate: 1 mL/min).

Peak 2: (1S,2R)-2-(5-(difluoromethyl)pyridin-2-yl)-N-(5-((2R*,4R*)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 2) ¹H NMR (400 MHz, DMSO-d6) δ (ppm): 10.96 (1H), 9.02-8.97 (1H), 8.60 (1H), 8.36-8.30 (1H), 8.00 (1H), 7.96 (1H), 7.93-7.83 (3H), 7.47 (1H), 7.08 (1H), 5.19-5.05 (3H), 4.99 (1H), 4.44 (1H), 4.15-4.03 (1H), 3.71-3.60 (1H), 2.77 (1H), 2.60 (1H), 2.26 (3H), 2.07 (1H), 1.93-1.76 (2H), 1.52-1.42 (1H). LCMS: 615 [M+H]⁺. Chiral HPLC: Rt=2.504 min (CHIRALPAK IF-3; Mobile Phase A: Hex (0.1% DEA): (EtOH:DCM=1:1)=65: 35; Flow rate: 1 mL/min).

Peak 3: (1S,2R)-2-(5-(difluoromethyl)pyridin-2-yl)-N-(5-((2R*,4R*)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 3) ¹H NMR (400 MHz, DMSO-d6) δ (ppm): 10.96 (1H), 9.00 (1H), 8.60 (1H), 8.38-8.30 (1H), 8.16 (1H), 7.98 (1H), 7.94-7.83 (3H), 7.47 (1H), 7.08 (1H), 5.19-5.05 (2H), 5.02 (1H), 4.89 (1H), 4.41 (1H), 3.87 (1H), 3.75-3.65 (1H), 2.83-2.73 (1H), 2.62-2.55 (1H), 2.38-2.32 (1H), 2.29 (3H), 1.86-1.76 (1H), 1.70 (1H), 1.54-1.43 (1H). LCMS: 615 [M+H]⁺. Chiral HPLC: Rt=2.707 min (CHIRALPAK IF-3; Mobile Phase A: Hex (0.1% DEA): (EtOH:DCM=1:1)=65: 35; Flow rate: 1 mL/min).

Peak 4: (1S,2R)-2-(5-(difluoromethyl)pyridin-2-yl)-N-(5-((2R*,4R*)-4-hydroxytetrahydrofuran-2-yl)-6-(3-methyl-1-(2,2,2-trifluoroethyl)-1H-pyrazol-4-yl)-[2,3′-bipyridin]-6′-yl)cyclopropane-1-carboxamide (Isomer 4) ¹H NMR (400 MHz, DMSO-d6) δ (ppm): 10.96 (1H), 9.00 (1H), 8.60 (1H), 8.37-8.32 (1H), 8.16 (1H), 7.98 (1H), 7.94-7.85 (3H), 7.47 (1H), 7.08 (1H), 5.20-5.06 (2H), 5.02 (1H), 4.89 (1H), 4.41 (1H), 3.87 (1H), 3.77-3.70 (1H), 2.84-2.71 (1H), 2.62-2.55 (1H), 2.38-2.33 (1H), 2.29 (3H), 1.86-1.77 (1H), 1.74-1.65 (1H), 1.52-1.42 (1H). LCMS: 615 [M+H]⁺. Chiral HPLC: Rt=3.909 min (CHIRALPAK IF-3; Mobile Phase A: Hex (0.1% DEA): (EtOH:DCM=1:1)=65: 35; Flow rate: 1 mL/min).

### Example 41. Synthesis of (1S,2R)—N-(5′-fluoro-5-((S)-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (I-178)

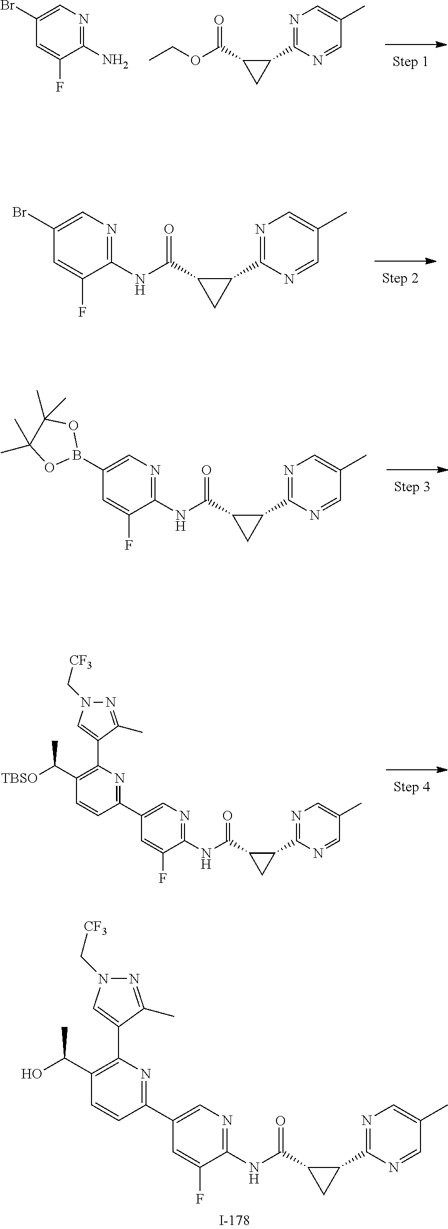

Step 1. Synthesis of (1S,2R)—N-(5-bromo-3-fluoropyridin-2-yl)-2-(5-methylpyrimidin-2-yl)cyclopropane-1-carboxamide. To a stirred solution of 5-bromo-3-fluoropyridin-2-amine (400 mg, 2.09 mmol) and ethyl (1S,2R)-2-(5-methylpyrimidin-2-yl)cyclopropane-1-carboxylate (475 mg, 2.30 mmol) in toluene (10 mL) was added LiHMDS (4.19 mL, 4.19 mmol, 1.0 M in THF) at 0° C. and the resulting mixture was stirred at 40° C. for 1 h. The reaction mixture was cooled, diluted with water (30 mL), extracted with EtOAc (3×30 mL), dried over Na₂SO₄, filtered, and concentrated the organics under reduced pressure. The residue was purified via reverse phase flash chromatography (C18 silica gel) using 20% to 25% ACN in water (with 10 mmol/L NH₄HCO₃) gradient to provide the title product. LCMS: 351 [M+H]⁺.

Step 2. Synthesis of (1S,2R)—N-(3-fluoro-5-(4,4,5,5-tetramethyl-1,3,2-dioxaborolan-2-yl)pyridin-2-yl)-2-(5-methylpyrimidin-2-yl)cyclopropane-1-carboxamide. To a stirred solution of (1S,2R)—N-(5-bromo-3-fluoropyridin-2-yl)-2-(5-methylpyrimidin-2-yl) cyclopropane-1-carboxamide (150 mg, 0.427 mmol) and 4,4,4′,4′,5,5,5′,5′-octamethyl-2,2′-bi(1,3,2-dioxaborolane) (434 mg, 1.71 mmol) in 1,4-dioxane (3 mL) were added AcOK (252 mg, 2.56 mmol) and Pd(dppf)Cl₂·CH₂Cl₂ (70 mg, 0.085 mmol) at RT and the mixture was stirred at 60° C. for 2 h under N₂ atmosphere. The reaction mixture was cooled, concentrated and purified via reverse phase flash chromatography (C18 silica gel) using 0% to 10% ACN in water (containing 0.1% FA) gradient to provide the title product. LCMS: 399 [M+H]⁺.

Step 3. Synthesis of (1S,2R)—N-(5-((S)-1-((tert-butyldimethylsilyl)oxy)ethyl)-5′-fluoro-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide. To a stirred solution of (1S,2R)—N-(3-fluoro-5-(4,4,5,5-tetramethyl-1,3,2-dioxaborolan-2-yl)pyridin-2-yl)-2-(5-methylpyrimidin-2-yl)cyclopropane-1-carboxamide (160 mg, 0.402 mmol) and (S)-3-(1-((tert-butyldimethylsilyl)oxy)ethyl)-6-chloro-2-(3-methyl-1-(2,2,2-trifluoroethyl)-1H-pyrazol-4-yl)pyridine (262 mg, 0.603 mmol) in 1,4-dioxane (4 mL) and H₂O (0.4 mL) were added K₃PO₄ (256 mg, 1.21 mmol) and Pd(dppf)Cl₂·CH₂Cl₂ (33 mg, 0.040 mmol) at RT and the mixture was stirred at 80° C. for 2 h under N₂ atmosphere. The reaction mixture was cooled, concentrated and purified via silica gel chromatography using a gradient of 0 to 10% MeOH in DCM to provide the title product. LCMS: 670 [M+H]⁺.

Step 4. Synthesis of (1S,2R)—N-(5′-fluoro-5-((S)-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide. A stirred solution of (1S,2R)—N-(5′-fluoro-5-((S)-1-hydroxyethyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (60 mg, 0.090 mmol) in THF (4 mL) was added Et₃N·3HF (144 mg, 0.900 mmol) at 0° C. and the mixture was stirred at 60° C. overnight. The reaction mixture was cooled, concentrated under reduced pressure and the residue was purified by Prep-HPLC (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient (B %): 19% B to 39% B in 10 min; Wave Length: 254 nm/220 nm; Rt (min): 8.36). The desired fractions were concentrated under reduced pressure. The residue was dissolved in ACN and H₂O and lyophilized to provide the title compound. ¹H NMR (400 MHz, DMSO-d6) δ (ppm): 10.56 (1H), 8.93-8.86 (1H), 8.48 (2H), 8.27-8.19 (1H), 8.11-7.98 (3H), 5.32 (1H), 5.19-5.09 (2H), 4.91-4.84 (1H), 2.81-2.73 (1H), 2.45-2.39 (1H), 2.29 (3H), 2.20 (3H), 1.91-1.83 (1H), 1.44-1.37 (1H), 1.34 (3H). LCMS: 556 [M+H]⁺.

### Example 42. Synthesis of (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1) & (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2) (I-179 & I-180)

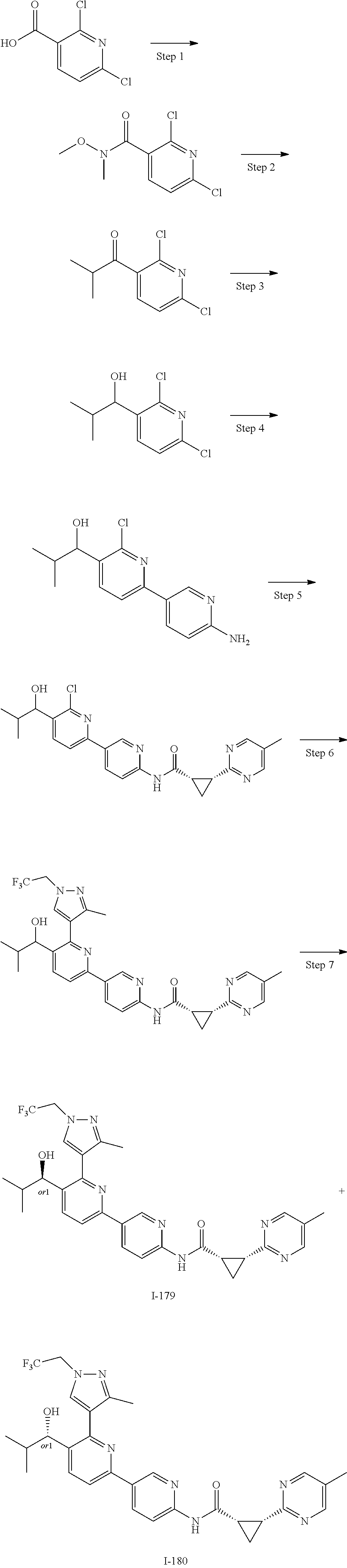

Step 1. Synthesis of 2,6-dichloro-N-methoxy-N-methylpyridine-3-carboxamide. To a stirred mixture of 2,6-dichloropyridine-3-carboxylic acid (30.0 g, 156 mmol) and N,O -dimethylhydroxylamine (14.3 g, 234 mmol) in THF (200 mL) were added HATU (59.4 g, 156 mmol) and DIEA (60.6 g, 468 mmol) at RT and the mixture was stirred at RT for 1 h. The resulting mixture was concentrated and purified via silica gel chromatography using a gradient of 0 to 50% EtOAc in petroleum ether to provide the title compound. LCMS: 235 [M+H]⁺.

Step 2. Synthesis of 1-(2,6-dichloropyridin-3-yl)-2-methylpropan-1-one. To a stirred mixture of 2,6-dichloro-N-methoxy-N-methylpyridine-3-carboxamide (15.6 g, 66.3 mmol) in THF (150 mL) was added bromo(isopropyl)magnesium (24.44 g, 165 mmol) at 0° C. and the mixture was stirred at RT for 1 h under N₂ atmosphere. The resulting mixture was concentrated and purified via silica gel chromatography using a gradient of 0 to 50% EtOAc in petroleum ether to provide the title compound. LCMS: 218 [M+H]⁺.

Step 3. Synthesis of (1S)-1-(2,6-dichloropyridin-3-yl)-2-methylpropan-1-ol. To a stirred mixture of 1-(2,6-dichloropyridin-3-yl)-2-methylpropan-1-one (2.1 g, 9.62 mmol) in methanol (20 mL) was added NaBH₄ (1.09 g, 28.8 mmol) at 0° C. and the mixture was stirred at RT for 1 h. The resulting mixture was concentrated and purified via silica gel chromatography using a gradient of 0 to 50% EtOAc in petroleum ether to provide the title compound. LCMS: 220 [M+H]⁺.

Step 4. Synthesis of 1-(6′-amino-6-chloro-[2,3′-bipyridin]-5-yl)-2-methylpropan-1-ol. To a stirred mixture of (1S)-1-(2,6-dichloropyridin-3-yl)-2-methylpropan-1-ol (800 mg, 3.63 mmol) and 6-aminopyridin-3-ylboronic acid (401 mg, 2.90 mmol) in Dioxane (10 mL) and H₂O (1 mL) were added Pd(dppf)Cl₂·CH₂Cl₂ (296 mg, 0.36 mmol) and K₂CO₃ (1.5 g, 10.9 mmol) at RT and the mixture was stirred at 80° C. for 1 h under N₂ atmosphere. The reaction mixture was cooled, concentrated and purified via silica gel chromatography using a gradient of 0 to 10% methanol in DCM to provide the title compound. LCMS: 278 [M+H]⁺.

Step 5. Synthesis of (1S,2R)—N-[6-chloro-5-(1-hydroxy-2-methylpropyl)-[2,3′-bipyridin]-6′-yl]-2-(5-methylpyrimidin-2-yl)cyclopropane-1-carboxamide. To a stirred mixture of 1-{6′-amino-6-chloro-[2,3′-bipyridin]-5-yl}-2-methylpropan-1-ol (150 mg, 0.54 mmol) and ethyl (1S,2R)-2-(5-methylpyrimidin-2-yl)cyclopropane-1-carboxylate (133 mg, 0.648 mmol) in THF (10 mL) was added LiHMDS (1.08 mL, 1.08 mmol, 1.0 M in THF) at 0° C. and the resulting mixture was stirred at 80° C. for 2 h under N₂ atmosphere. The reaction mixture was cooled, concentrated and purified via reverse phase flash chromatography (C18 silica gel) using 20% to 30% ACN in water (with 10 mmol/L NH₄HCO₃) gradient to provide the title compound. LCMS: 438 [M+H]⁺.

Step 6. Synthesis of (1S,2R)—N-[5-(1-hydroxy-2-methylpropyl)-6-[3-methyl-1-(2,2,2-trifluoroethyl)pyrazol-4-yl]-[2,3′-bipyridin]-6′-yl]-2-(5-methylpyrimidin-2-yl)cyclopropane-1-carboxamide. To a stirred mixture of (1S,2R)—N-[6-chloro-5-(1-hydroxy-2-methylpropyl)-[2,3′-bipyridin]-6′-yl]-2-(5-methylpyrimidin-2-yl)cyclopropane-1-carboxamide (70 mg, 0.16 mmol) and 3-methyl-4-(4,4,5,5-tetramethyl-1,3,2-dioxaborolan-2-yl)-1-(2,2,2-trifluoroethyl)pyrazolidine (56 mg, 0.19 mmol) in Dioxane (3 mL) and H₂O (0.3 mL) were added K₂CO₃ (44 mg, 0.32 mmol) and Pd(dppf)Cl₂·CH₂Cl₂ (13 mg, 0.01 mmol) at 0° C. and the resulting mixture was stirred at 80° C. for 2 h under N₂ atmosphere. The reaction mixture was cooled, concentrated and purified via reverse phase flash chromatography (C18 silica gel) using 20% to 30% ACN in water (with 10 mmol/L NH₄HCO₃) gradient to provide the title compound. LCMS: 566 [M+H]⁺.

Step 7. Separation of (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1) & (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2). (1S,2R)—N-(5-(1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (45 mg, 0.08 mmol) was purified by Chiral-HPLC (Column: JW-CHIRALPAK IK, 20×250 mm, 5 μm; Mobile Phase A: EtOH:DCM=1:1, Mobile Phase B: Hex (0.1% 2M NH₃-MeOH); Flow rate: 20 mL/min; Gradient (B %): 70% B to 70% B in 11 min % B; Wave Length: 220/254 nm; RT1(min): 7.33; RT2(min): 9.85, Sample Solvent: EtOH:DCM=1:1; Injection Volume: 0.9 mL; Number Of Runs: 6). The residue was dissolved in ACN and H₂O and lyophilized to provide the title compounds as peak 1 (Isomer 1) and peak 2 (Isomer 2).

Peak 1: (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 1) ¹H NMR (400 MHz, DMSO-d6) δ (ppm): 10.89 (1H), 8.99 (1H), 8.50 (2H), 8.37-8.30 (1H), 7.98-7.86 (4H), 5.23-5.09 (3H), 4.45 (1H), 2.82-2.71 (1H), 2.53-2.52 (1H), 2.21 (6H), 1.89-1.81 (1H), 1.80-1.73 (1H), 1.44-1.35 (1H), 0.85 (3H), 0.65 (3H). LCMS: 566 [M+H]⁺. Chiral HPLC: Rt=1.738 min (Column: CHIRALPAKIG IK-3; Mobile Phase A: Hexane (0.1% DEA): (EtOH:DCM=1:1)=70: 30; Flow rate: 1 mL/min).

Peak 2: (1S,2R)—N-(5-((R*)-1-hydroxy-2-methylpropyl)-6-(3-methyl-1-(2,2,2-trifluoroethyl)-1H-pyrazol-4-yl)-[2,3′-bipyridin]-6′-yl)-2-(5-methylpyrimidin-2-yl)cyclopropane-1-carboxamide (Isomer 2) ¹H NMR (400 MHz, DMSO-d6) δ (ppm): 10.88 (1H), 9.02-8.95 (1H), 8.49 (2H), 8.37-8.30 (1H), 8.02-7.82 (4H), 5.23 (1H), 5.21-5.09 (2H), 4.49-4.39 (1H), 2.82-2.71 (1H), 2.52-2.52 (1H), 2.21 (6H), 1.88-1.81 (1H), 1.79-1.73 (1H), 1.44-1.34 (1H), 0.85 (3H), 0.66 (3H). LCMS: 566 [M+H]⁺. Chiral HPLC: Rt=2.353 min (Column: CHIRALPAKIG IK-3; Mobile Phase A: Hexane (0.1% DEA): (EtOH:DCM=1:1)=70:30; Flow rate: 1 mL/min).

### Example 44. Synthesis of (1S,2R)-2-[5-(difluoromethyl)pyridin-2-yl]-N-{5-[(1S)-1-hydroxyethyl]-6-[3-methyl-1-(2,2,2-trifluoroethyl)-1H-pyrazol-4-yl]-[2,3′-bipyridin]-6′-yl}cyclopropane-1-carboxamide (I-7)

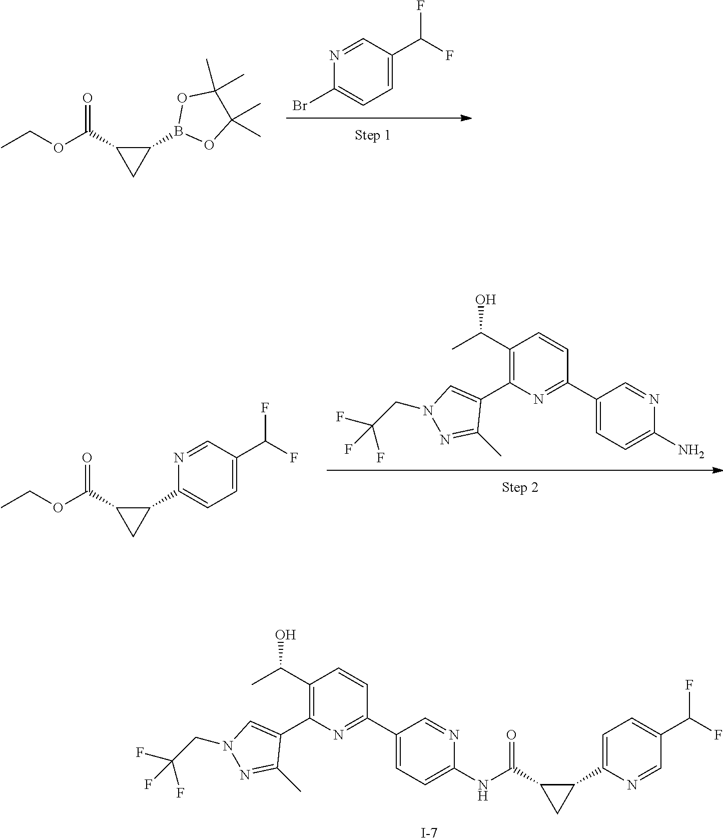

### Step 1. Synthesis of ethyl (1S,2R)-2-(5-(difluoromethyl)pyridin-2-yl)cyclopropane-1-carboxylate

To a solution of 2-bromo-5-(difluoromethyl)pyridine (100.00 g, 480.74 mmol, 1.00 eq), ethyl (1S,2R)-2-(4,4,5,5-tetramethyl-1,3,2-dioxaborolan-2-yl)cyclopropane-carboxylate (121.20 g, 504.78 mmol, 1.05 eq) in toluene (800 mL) and H₂O (80 mL) was added Cs₂CO₃ (469.18 g, 1.44 mol, 3.00 eq) and cataCXium® A Pd G3 (28.01 g, 38.46 mmol, 0.08 eq). The reaction mixture was stirred at 100° C. for 16 h under N₂ atmosphere. The reaction mixture was cooled down to room temperature, filtered and the filtrate was concentrated under reduced pressure to give a residue. To the residue was added H₂O (100 mL) and the resulting mixture was extracted with EtOAc (250 mL*2). The combined organic layers were washed with brine (300 mL), dried over anhydrous Na₂SO₄, filtered and the filtrate was concentrated under reduced pressure to give a residue. The residue was purified by flash silica gel chromatography (Eluent of EtOAc/PE) to afford the title compound. ¹H NMR (400 MHz, CDCl₃) δ=8.63 (s, 1H), 7.76 (d, 1H), 7.38 (d, 1H), 6.69 (t, 1H), 4.01-3.87 (m, 2H), 2.85-2.67 (m, 1H), 2.29-2.19 (m, 1H), 1.94-1.82 (m, 1H), 1.55-1.44 (m, 1H), 1.06 (t, 3H) LCMS: 242.0 [M+H]⁺

### Step 2. Synthesis of (1S,2R)-2-[5-(difluoromethyl)pyridin-2-yl]-N-{5-[(1S)-1-hydroxyethyl]-6-[3-methyl-1-(2,2,2-trifluoroethyl)-1H-pyrazol-4-yl]-[2,3′-bipyridin]-6′-yl}cyclopropane-1-carboxamide

To a solution of (1S)-1-[6-(6-amino-3-pyridyl)-2-[3-methyl-1-(2,2,2-trifluoroethyl)pyrazol-4-yl]-3-pyridyl]ethanol (prepared as described in step 6 of I-152 synthesis) (10.00 g, 26.50 mmol, 1.0 eq) in THF (50 mL) was added AlMe₃ (2 M in toluene, 66.25 mL, 5.0 eq) at 0° C. under N₂ atmosphere. The resulting mixture was stirred at 40° C. for 1 h. Then ethyl (1S,2R)-2-[5-(difluoromethyl)-2-pyridyl]cyclopropanecarboxylate (7.35 g, 30.47 mmol, 1.15 eq) in THF (50 mL) was added to the mixture at 20° C. under N₂ atmosphere. The resulting mixture was stirred at 90° C. for another 15 h. The reaction mixture was cooled to 0° C. and quenched with sat. aq. NH₄Cl (100 mL) under N₂ atmosphere. The resulting mixture was filtered and the filter cake was washed with DCM/MeOH (10/1, 200 mL). The combined filtrate was extracted with DCM (3*100 mL). The combined organic layers were washed with brine (100 mL), dried over anhydrous Na₂SO₄, filtered and the filtrate was concentrated under reduced pressure. The residue was purified by flash silica gel chromatography (Eluent of MeOH/DCM) to afford the title compound. ¹H NMR (400 MHz, CD₃OD) δ=8.91 (d, 1H), 8.56 (s, 1H), 8.29-8.24 (m, 1H), 8.11 (d, 1H), 7.93 (s, 1H), 7.89 (d, 2H), 7.85-7.80 (m, 1H), 7.55 (d, 1H), 7.03-6.68 (m, 1H), 4.99-4.92 (m, 3H), 2.89-2.72 (m, 1H), 2.52 (d, 1H), 2.32 (s, 3H), 2.01-1.91 (m, 1H), 1.62-1.50 (m, 1H), 1.41 (d, 3H). LCMS: 573.3 [M+H]⁺

### Example 45. Synthesis of (1S,2R)—N-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-2-[5-(trifluoromethyl)pyrazin-2-yl]cycopropane-1-carboxamide (I-347)

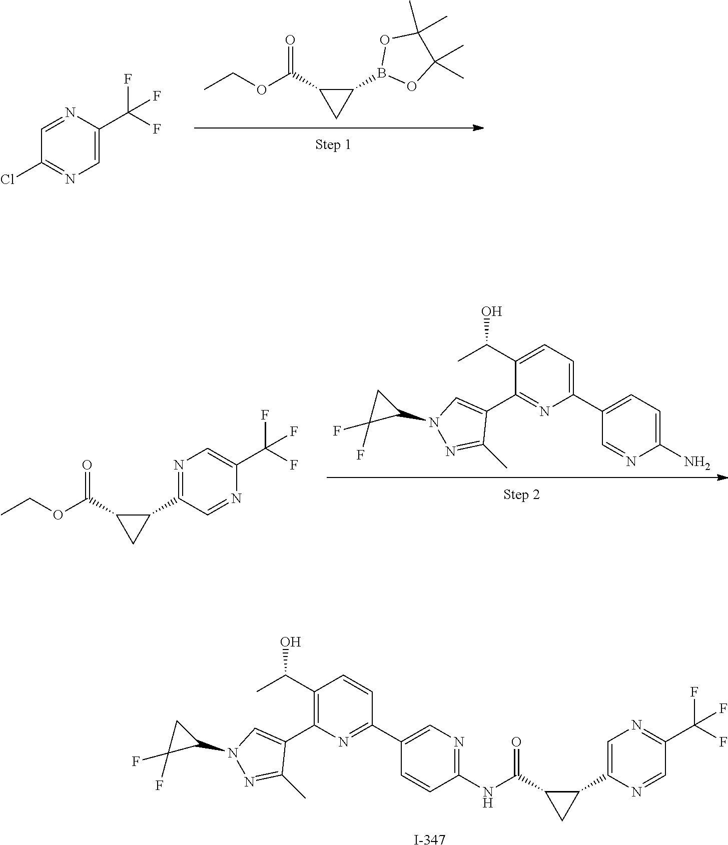

### Step 1. Synthesis of ethyl (1S,2R)-2-(5-(trifluoromethyl)pyrazin-2-yl)cyclopropane-1-carboxylate

A mixture of 2-chloro-5-(trifluoromethyl)pyrazine (1.00 g, 5.48 mmol, 1 eq), ethyl (1S,2R)-2-(4,4,5,5-tetramethyl-1,3,2-dioxaborolan-2-yl)cyclopropanecarboxylate (1.38 g, 5.75 mmol, 1.05 eq), cataCXium® A Pd G3, (319 mg, 438 mol, 0.08 eq), Cs₂CO₃ (5.36 g, 16.44 mmol, 3 eq) in toluene (15 mL) and H₂O (1.5 mL) was degassed and purged with N₂ for 3 times, and then the mixture was stirred at 100° C. for 2 h under N₂ atmosphere. The reaction mixture was filtered and the filtrate was concentrated under reduced pressure to give a residue. The residue was purified by flash silica gel chromatography (Eluent of EtOAc/PE) to afford the title compound. ¹H NMR (400 MHz, CDCl₃) δ=8.83 (s, 1H), 8.67 (s, 1H), 4.08-3.88 (m, 2H), 2.83-2.68 (m, 1H), 2.39-2.24 (m, 1H), 2.02-1.92 (m, 1H), 1.62-1.58 (m, 1H), 1.09 (t, 3H). LCMS: 261.1 [M+H]⁺

### Step 2. Synthesis of (1S,2R)—N-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-2-[5-(trifluoromethyl)pyrazin-2-yl]cyclopropane-1-carboxamide

To a solution of (S)-1-(6′-amino-6-(1-((R)-2,2-difluorocyclopropyl)-3-methyl-1H-pyrazol-4-yl)-[2,3′-bipyridin]-5-yl)ethan-1-ol (prepared as described in step 1 of I-164 synthesis) (71 mg, 192 μmol, 1 eq) and ethyl (1S,2R)-2-(5-(trifluoromethyl)pyrazin-2-yl)cyclopropane-1-carboxylate (50 mg, 192 mol, 1 eq) in THF (1.5 mL) was added AlMe₃ (2 M in toluene, 576 L, 6 eq) at 20° C. under N₂ atmosphere. The resulting mixture was stirred at 60° C. for 12 h under N₂ atmosphere. The reaction mixture was quenched by addition of sat. aq. NH₄Cl (5 mL) at 20° C., then diluted with water (4 mL) and extracted with DCM (10 mL*3). The combined organic layers were washed with brine (5 mL), dried over anhydrous Na₂SO₄, filtered and the filtrate was concentrated under reduced pressure. The residue was purified by reverse phase HPLC (C18 column, water (0.1% NH₄HCO₃)-ACN) to give the title compound. ¹H NMR (400 MHz, DMSO-d₆) δ=11.07 (s, 1H), 9.02 (s, 1H), 8.98 (d, 1H), 8.85 (s, 1H), 8.37-8.28 (m, 1H), 8.06-7.99 (m, 2H), 7.90 (d, 1H), 7.84 (d, 1H), 5.26 (d, 1H), 4.92-4.78 (m, 1H), 4.61-4.47 (m, 1H), 2.94-2.81 (m, 1H), 2.74-2.65 (m, 1H), 2.47-2.39 (m, 1H), 2.37-2.28 (m, 1H), 2.24 (s, 3H), 2.00-1.88 (m, 1H), 1.63-1.53 (m, 1H), 1.30 (d, 3H). LCMS: 586.2 [M+H]⁺

### Example 46. Synthesis of (1S,2R)—N-(6-{1-[(1R*)-2,2-difluorocyclopropyl]-4-methyl-1H-pyrazol-3-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 1) (I-192) and (1S,2R)—N-(6-{1-[(1R*)-2,2-difluorocyclopropyl]-4-methyl-1H-pyrazol-3-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 2) (I-193)

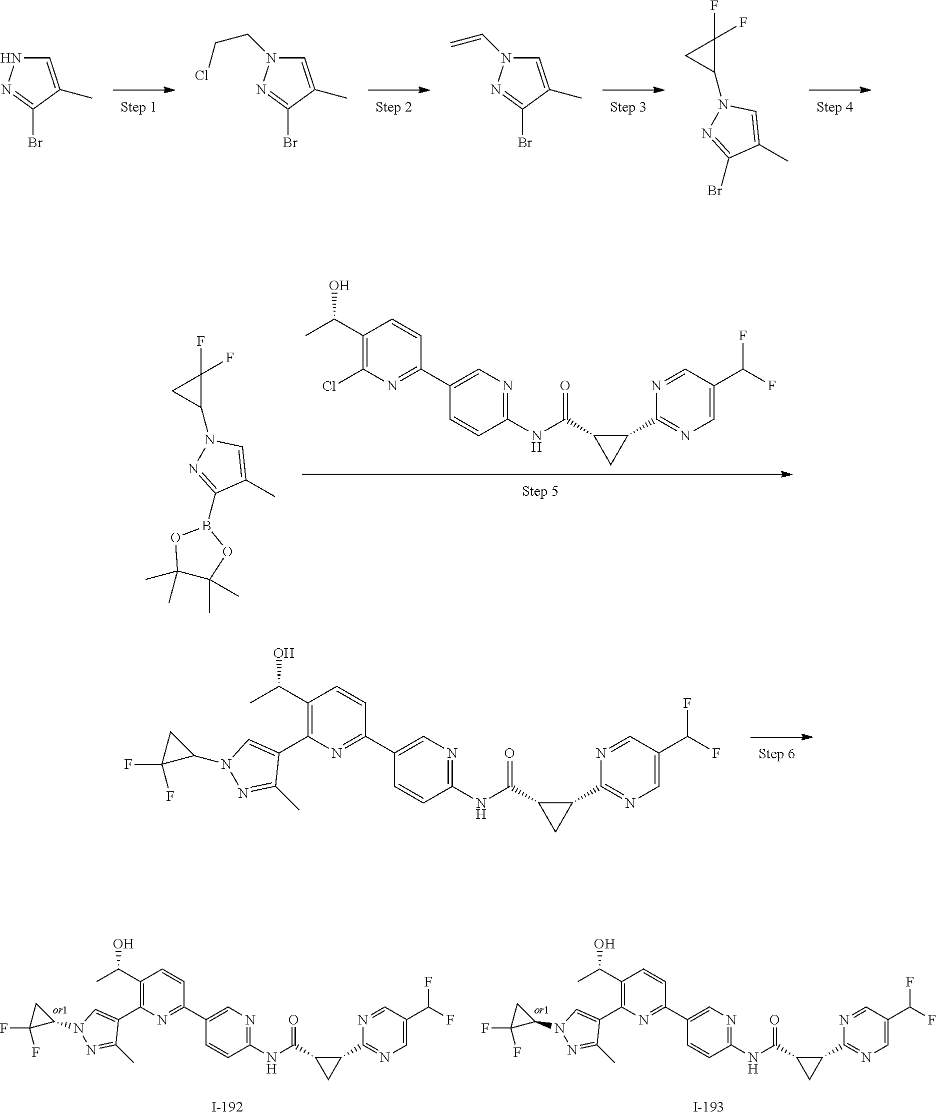

### Step 1. Synthesis of 2-chloro-5-(difluoromethyl)pyrimidine and 5-bromo-1-(2-chloroethyl)-4-methyl-1H-pyrazole

A mixture of 3-bromo-4-methyl-1H-pyrazole (5 g, 0.06 mmol), 1-bromo-2-chloroethane (6.7 g, 0.09 mmol) and Cs₂CO₃ (15.2 mg, 0.09 mmol) in 1,4-dioxane (50 mL) was stirred at 100° C. overnight. The resulting mixture was allowed to cool down to room temperature, concentrated under reduced pressure, diluted with water (200 mL) and extracted with EtOAc (3*200 mL). The combined organic layers were washed with brine (3*50 mL), dried over Na₂SO₄, filtered and the filtrate was concentrated under reduced pressure to afford the title compound as a ~5.1 mixture with undesired regioisomer. The resulting product was used in the next step directly without further purification. LCMS: 223 and 225 [M+H]⁺

### Step 2. Synthesis of 3-bromo-1-ethenyl-4-methylpyrazole

A mixture of 3-bromo-1-(2-chloroethyl)-4-methylpyrazole and its regioisomer obtained in step 1 (4 g, 9.396 mmol), benzyl triethyl ammonium chloride (220 mg, 0.483 mmol) and NaOH (1.12 g, 14.1 mmol) in EtOH (32 mL) and H₂O (80 mL) was stirred at 80° C. overnight. The reaction mixture was allowed to cool to room temperature, diluted with water (200 mL) and extracted with EtOAc (200 mL*3). The combined organic layers were washed with brine, dried over Na₂SO₄, filtered and the filtrate was concentrated under reduced pressure. The residue was purified by silica gel column chromatography (Eluent of PE/EtOAc) to afford the title compound. LCMS: 187 and 189 [M+H]⁺.

### Step 3. Synthesis of 3-bromo-1-(2,2-difluorocyclopropyl)-4-methylpyrazole

To a stirred solution of 3-bromo-1-ethenyl-4-methylpyrazole (2 g, 1.87 mmol) and NaI (1.12 g, 7.48 mmol) in MeCN (20 mL) was added trifluoromethyltrimethylsilane (1.33 g, 9.36 mmol) at room temperature under N₂ atmosphere. The resulting mixture was stirred at room temperature overnight under N₂ atmosphere and then concentrated under reduced pressure. The residue was purified by prep-TLC (Eluent of PE/EtOAc) to afford the title compound. LCMS: 237 and 239 [M+H]⁺.

### Step 4. Synthesis of 1-(2,2-difluorocyclopropyl)-4-methyl-3-(4,4,5,5-tetramethyl-1,3,2-dioxaborolan-2-yl)-1H-pyrazole

To a solution of 3-bromo-1-(2,2-difluorocyclopropyl)-4-methylpyrazole (1 g, 4.22 mmol) in toluene (20 mL) were added TEA (1.28 g, 12.6 mmol), XPhos Pd G2 (0.33 g, 0.422 mmol) and 4,4,5,5-tetramethyl-1,3,2-dioxaborolane (1.62 g, 12.6 mmol) at room temperature under N₂ atmosphere. The resulting mixture was stirred at 100° C. for 1 h under N₂ atmosphere. The reaction mixture was allowed to cool to room temperature. The resulting mixture was filtered and the filter cake was washed with EtOAc (3*50 mL). The combined filtrate was concentrated under reduced pressure to afford the title compound, which was used in the next step directly without further purification. LCMS: 285 [M+H]⁺.

### Step 5. Synthesis of (1S,2R)—N-(6-[1-(2,2-difluorocyclopropyl)-4-methylpyrazol-3-yl]-5-[(1S)-1-hydroxyethyl]-[2,3′-bipyridin]-6′-yl-2-[5-(difluoromethyl)pyrimidin-2-yl]cyclopropane-1-carboxamide

To a stirred mixture of (1S,2R)—N-(6-chloro-5-[(1S)-1-hydroxyethyl]-[2,3′-bipyridin]-6′-yl-2-[5-(difluoromethyl)pyrimidin-2-yl]cyclopropane-1-carboxamide (obtained as described in step 4 of I-150 synthesis) (140 mg, 0.94 mmol), 1-(2,2-difluorocyclopropyl)-4-methyl-3-(4,4,5,5-tetramethyl-1,3,2-dioxaborolan-2-yl)pyrazole (1.91 g, 2.83 mmol) and K₂CO₃ (130 mg, 2.83 mmol) in 1,4-dioxane (5 mL) and H₂O (0.5 mL) was added Pd(PPh₃)₄(73 mg, 0.18 mmol) at room temperature under N₂ atmosphere. The resulting mixture was stirred at 80° C. for 1 h under N₂ atmosphere. The reaction mixture was allowed to cool down to room temperature, diluted with water (30 mL) and extracted with EtOAc (3*50 mL). The combined organic layers were washed with brine, dried over Na₂SO₄, filtered and the filtrate was concentrated under reduced pressure. The residue was purified using prep-TLC (Eluent of CH₂Cl₂/MeOH) to afford the title compound. LCMS: 568 [M+H]⁺

### Step 6. Separation of (1S,2R)—N-(6-(1-(2,2-difluorocyclopropyl)-4-methyl-1H-pyrazol-3-yl)-5-((S)-1-hydroxyethyl)-[2,3′-bipyridin]-6′-yl)-2-(5-(difluoromethyl)pyrimidin-2-yl)cyclopropane-1-carboxamide

The mixture of stereoisomers of (1S,2R)—N-(6-[1-(2,2-difluorocyclopropyl)-4-methylpyrazol-3-yl]-5-[(1S)-1-hydroxyethyl]-[2,3′-bipyridin]-6′-yl-2-[5-(difluoromethyl)pyrimidin-2-yl]cyclopropane-1-carboxamide (78 mg, 0.123 mmol) was separated by Prep-Chiral-HPLC (Column: CHIRALPAK® IK, 5 μm, 20 mm×250 mm; Mobile Phase A: EtOH:CH₂Cl₂=1:1, Mobile Phase B: Hex (with 0.5% NH₃ (2 M in MeOH)); Flow rate: 20 mL/min; Gradient (B %) 70% B; Wave Length: 220/254 nm; Rt (peak1): 11.43 min; Rt (peak2): 13.91 min; sample solvent: EtOH:CH₂Cl₂=1:1) to afford the title compounds.

Peak 1 (first eluting): (1S,2R)—N-(6-{1-[(1R*)-2,2-difluorocyclopropyl]-4-methyl-1H-pyrazol-3-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 1) (I-192). ¹H NMR (400 MHz, DMSO-d₆) δ 10.96 (s, 1H), 9.00 (d, J=2.6 Hz, 1H), 8.90 (s, 2H), 8.37-8.28 (m, 1H), 8.11 (d, J=8.4 Hz, 1H), 8.03-7.91 (m, 1H), 7.89-7.76 (m, 2H), 7.33-6.97 (m, 1H), 5.60-5.10 (m, 2H), 4.87-4.52 (m, 1H), 3.00-2.82 (m, 1H), 2.64-2.57 (m, 1H), 2.43-2.26 (m, 2H), 2.19 (d, J=13.1 Hz, 3H), 1.90 (d, J=6.4 Hz, 1H), 1.55-1.42 (m, 1H), 1.38-1.27 (m, 3H). LCMS: 568 [M+H]⁺; Analytical chiral HPLC: Rt=2.921 min.

Peak 2 (second eluting): (1S,2R)—N-(6-{1-[(1R*)-2,2-difluorocyclopropyl]-4-methyl-1H-pyrazol-3-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 2) (I-193). ¹H NMR (400 MHz, DMSO-d₆) δ 10.96 (s, 1H), 9.00 (d, J=2.4 Hz, 1H), 8.90 (s, 2H), 8.39-8.30 (m, 1H), 8.12 (d, J=8.2 Hz, 1H), 7.96 (d, J=8.3 Hz, 1H), 7.92-7.78 (m, 2H), 7.33-7.01 (m, 1H), 5.21 (s, 2H), 4.59 (d, J=11.7 Hz, 1H), 2.91 (d, J=8.4 Hz, 1H), 2.61 (d, J=7.5 Hz, 1H), 2.44-2.31 (m, 2H), 2.18 (s, 3H), 1.91 (s, 1H), 1.49 (d, J=4.2 Hz, 1H), 1.32 (s, 3H). LCMS: 568 [M+H]⁺. Analytical chiral HPLC: Rt=3.707 min.

Analytical chiral HPLC method: Column: CHIRALPAKIG IK-3; Mobile Phase A: Hexa ne (0.1% DEA): (EtOH:DCM=1:1)=70:30; Flow rate: 1 mL/min.

### Example 47. Synthesis of (1S,2R)-2-[5-(difluoromethyl)pyrimidin-2-yl]-N-(6-{1-[(1-flu orocyclopropyl)methyl]-3-methyl-1H-pyrazol-4-yl}-5-[(1S)-1-hydroxyethyl]-[2,3′-bipyridin]-6′-yl)cyclopropane-1-carboxamide (I-439) and (1S,2R)-2-[5-(difluoromethyl)pyrimidin-2-yl]-N-(6-{1-[(1-fluorocyclopropyl)methyl]-5-methyl-1H-pyrazol-4-yl}-5-[(1S)-1-hydroxyethyl]-[2,3′-bipyridin]-6′-yl)cyclopropane-1-carboxamide (I-442)

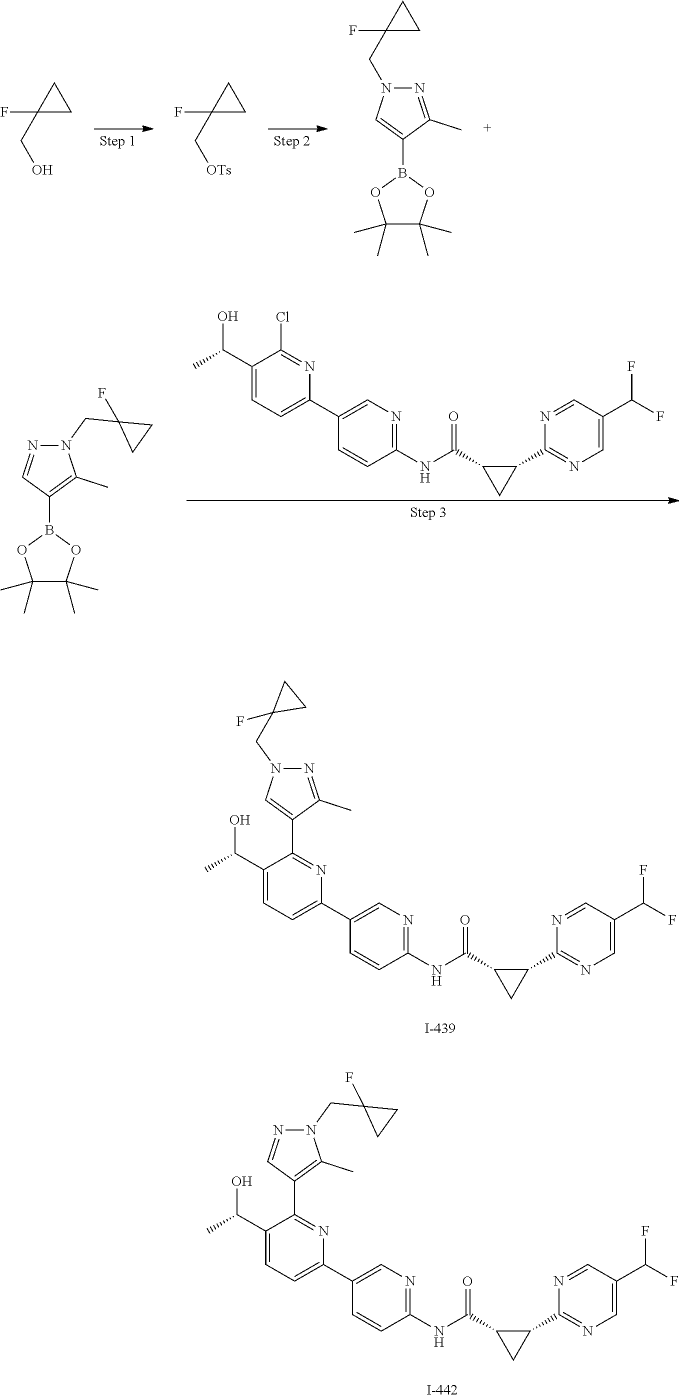

### Step 1. Synthesis of (1-fluorocyclopropyl)methyl 4-methylbenzenesulfonate

To a stirred solution of (1-fluorocyclopropyl)methanol (10 g, 110 mmol), TEA (56.1 g, 554 mmol) and DMAP (2.7 g, 22.1 mmol) in CH₂Cl₂ (80 mL) was added tosyl chloride (31.7 g, 166 mmol) at 0° C. The resulting mixture was stirred at room temperature for 2 h. The reaction mixture was poured into water and concentrated under reduced pressure. The residue was purified by silica gel column chromatography (Eluent of PE/EtOAc) to provide the title compound. No MS signal was observed and the compound was used as such in the next step.

### Step 2. Synthesis of 1-((1-fluorocyclopropyl)methyl)-3-methyl-4-(4,4,5,5-tetramethyl-1,3,2-dioxaborolan-2-yl)-1H-pyrazole and 1-((1-fluorocyclopropyl)methyl)-5-methyl-4-(4,4,5,5-tetramethyl-1,3,2-dioxaborolan-2-yl)-1H-pyrazole

A solution of 3-methyl-4-(4,4,5,5-tetramethyl-1,3,2-dioxaborolan-2-yl)-1H-pyrazole (6 g, 28.8 mmol) in MeCN (50 mL) was treated with (1-fluorocyclopropyl)methyl 4-methylbenzenesulfonate (21.1 g, 86.5 mmol) and Cs₂CO₃ (28.1 g, 86.5 mmol) at room temperature. The resulting mixture was stirred at room temperature overnight and then concentrated under reduced pressure. The residue was purified by silica gel column chromatography (Eluent of PE/EtOAc) to provide a mixture of the title isomers (~2:1). LCMS: 281 [M+H]⁺

### Step 3. Synthesis of (1S,2R)-2-[5-(difluoromethyl)pyrimidin-2-yl]-N-(6-{1-[(1-fluorocyclopropyl)methyl]-3-methyl-1H-pyrazol-4-yl}-5-[(1S)-1-hydroxyethyl]-[2,3′-bipyridin]-6′-yl)cyclopropane-1-carboxamide and (1S,2R)-2-[5-(difluoromethyl)pyrimidin-2-yl]-N-(6-{1-[(1-fluorocyclopropyl)methyl]-5-methyl-1H-pyrazol-4-yl}-5-[(1S)-1-hydroxyethyl]-[2,3′-bipyridin]-6′-yl)cyclopropane-1-carboxamide

To a stirred solution of (1S,2R)—N-(6-chloro-5-((S)-1-hydroxyethyl)-[2,3′-bipyridin]-6′-yl)-2-(5-(difluoromethyl)pyrimidin-2-yl)cyclopropane-1-carboxamide (obtained as described in step 4 of I-150 synthesis) (1 g, 2.24 mmol) and a mixture of 1-((1-fluorocyclopropyl)methyl)-3-methyl-4-(4,4,5,5-tetramethyl-1,3,2-dioxaborolan-2-yl)-1H-pyrazole and 1-((1-fluorocyclopropyl)methyl)-5-methyl-4-(4,4,5,5-tetramethyl-1,3,2-dioxaborolan-2-yl)-1H-pyrazole from Step 2 (1.3 g, 4.48 mmol) in dioxane (15 mL) and H₂O (1.5 mL) were added K₂CO₃ (929 mg, 6.72 mmol) and Pd(PPh₃)₄(518 mg, 0.449 mmol) at room temperature under N₂ atmosphere. The resulting mixture was stirred at 100° C. overnight under N₂ atmosphere. The reaction mixture was diluted with water (30 mL) and extracted with EtOAc (3*50 mL). The combined organic layers were washed with brine, dried over Na₂SO₄, filtered and the filtrate was concentrated under reduced pressure. The residue was purified by Prep-TLC (Eluent of CH₂Cl₂/MeOH) followed by preparative chiral HPLC (Column: CHIRALPAK® IM, 5 m, 20 mm*250 mm; Mobile Phase A: EtOH: CH₂Cl₂=1:1, Mobile Phase B: Hex (with 0.5% NH₃ (2 M in MeOH)); Flow rate: 20 mL/min; Gradient (B %) 65% B; Wave Length: 220/254 nm; R^{†}=6.71 and 9.46 min; Sample solvent: EtOH: CH₂Cl₂=1:1) to afford the title compounds. (1S,2R)-2-(5-(difluoromethyl)pyrimidin-2-yl)-N-(6-(1-((1-fluorocyclopropyl)methyl)-3-methyl-1H-pyrazol-4-yl)-5-((S)-1-hydroxyethyl)-[2,3′-bipyridin]-6′-yl)cyclopropane-1-carboxamide (I-x4) was further purified by Prep-HPLC with the following conditions (Column: XBridge Prep OBD C18 Column, 30*150 mm, 5 m; Mobile Phase A: Water (10 mmol/L-NH₄HCO₃), Mobile Phase B: ACN; Flow rate: 60 mL/min; Gradient (B %): 27% B to 39% B in 8 min; Wave Length: 254 nm/220 nm; Rt=8.07 min)) to provide the title compound.

(1S,2R)-2-[5-(difluoromethyl)pyrimidin-2-yl]-N-(6-{1-[(1-fluorocyclopropyl)methyl]-3-methyl-1H-pyrazol-4-yl}-5-[(1S)-1-hydroxyethyl]-[2,3′-bipyridin]-6′-yl)cyclopropane-1-carboxamide (I-439): ¹H NMR (400 MHz, DMSO-d₆) δ 10.97 (s, 1H), 9.03-8.97 (m, 1H), 8.90 (t, J=1.4 Hz, 2H), 8.37-8.30 (m, 1H), 8.03 (d, J=8.3 Hz, 1H), 7.94 (s, 1H), 7.92-7.83 (m, 2H), 7.17 (t, J=55.0 Hz, 1H), 5.26 (s, 1H), 4.91 (d, J=6.8 Hz, 1H), 4.52 (d, J=21.9 Hz, 2H), 2.96-2.86 (m, 1H), 2.66-2.52 (m, 1H), 2.28 (s, 3H), 1.95-1.86 (m, 1H), 1.53-1.44 (m, 1H), 1.34 (d, J=6.3 Hz, 3H), 1.16-1.04 (m, 2H), 1.01-0.90 (m, 2H). LCMS: 564 [M+H]⁺. Analytical chiral HPLC: Rt=2.816 min.

(1S,2R)-2-[5-(difluoromethyl)pyrimidin-2-yl]-N-(6-{1-[(1-fluorocyclopropyl)methyl]-5-methyl-1H-pyrazol-4-yl}-5-[(1S)-1-hydroxyethyl]-[2,3′-bipyridin]-6′-yl)cyclopropane-1-carboxamide (I-442): ¹H NMR (400 MHz, DMSO-d₆) δ 10.96 (s, 1H), 9.03-8.95 (m, 1H), 8.89 (d, J=1.3 Hz, 2H), 8.36-8.29 (m, 1H), 8.04 (d, J=8.2 Hz, 1H), 7.95-7.83 (m, 2H), 7.64 (s, 1H), 7.17 (t, J=55.0 Hz, 1H), 5.26 (d, J=4.3 Hz, 1H), 4.96-4.86 (m, 1H), 4.67-4.49 (m, 2H), 2.98-2.85 (m, 1H), 2.64-2.56 (m, 1H), 2.40 (s, 3H), 1.93-1.86 (m, 1H), 1.53-1.43 (m, 1H), 1.33 (d, J=6.3 Hz, 3H), 1.21-1.04 (m, 2H), 1.07-0.93 (m, 2H). LCMS: 564 [M+H]⁺. Analytical chiral HPLC: Rt=4.178 min.

Analytical chiral HPLC method: Column: CHIRALPAK IF-3; Mobile Phase A: Hex (0.1% DEA): (MeOH: CH₂Cl₂=1:1)=50:50; Flow rate: 1 mL/min.

### Example 48. Synthesis of (1S,2R)-2-[5-(difluoromethyl)pyridin-2-yl]-N-{5-[(1S)-1-hydroxyeth yl]-4′-(methylamino)-6-(methylsulfanyl)-[2,3′-bipyridin]-6′-yl}cyclopropane-1-carboxamide (I-4 83)

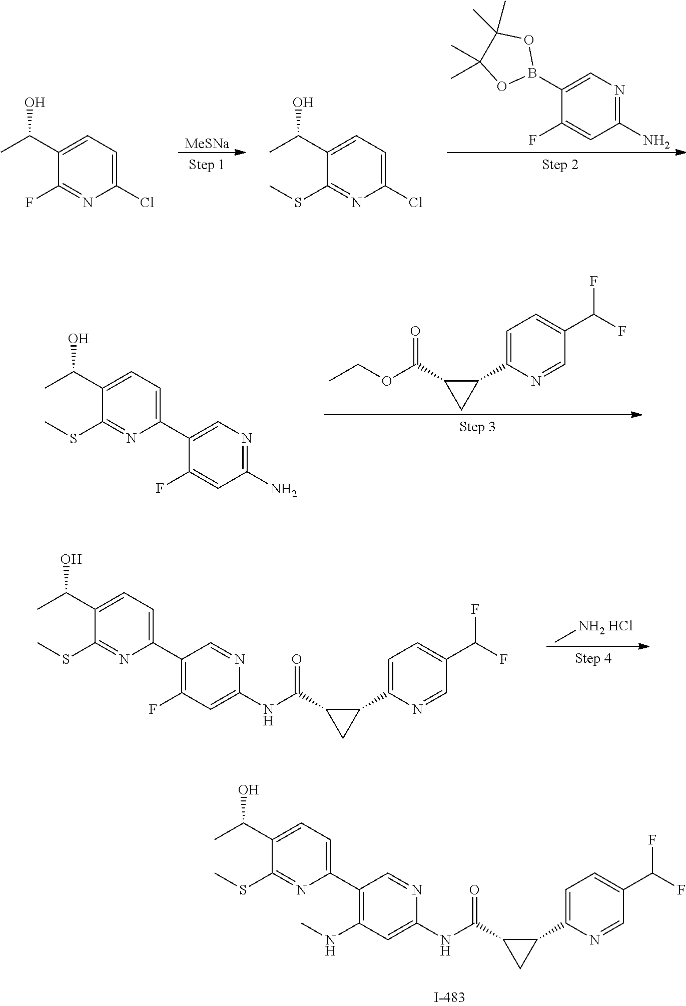

### Step 1. Synthesis of (S)-1-(6-chloro-2-(methylthio)pyridin-3-yl)ethan-1-ol

To a solution of (1S)-1-(6-chloro-2-fluoro-3-pyridyl)ethanol (prepared as described in step 1 of I-11 synthesis) (500 mg, 2.85 mmol, 1 eq) in DMF (10 mL) was added NaSMe (240 mg, 3.42 mmol, 1.2 eq) at 20° C. under N₂. The resulting mixture was stirred at 20° C. for 3 hours. The reaction mixture was diluted with water (50 ml) and then extracted with EtOAc (15 mL*3). The combined organic layers were washed with brine (10 mL*2), dried over anhydrous Na₂SO₄, filtered and the filtrate was concentrated under reduced pressure. The residue was purified by silica gel chromatography (Eluent of PE/EtOAc) to afford the title compound. LCMS: 204.0 [M+H]⁺

### Step 2. Synthesis of (S)-1-(6′-amino-4′-fluoro-6-(methylthio)-[2,3′-bipyridin]-5-yl)ethan-1-ol

To a solution of (1S)-1-(6-chloro-2-methylsulfanyl-3-pyridyl)ethanol (427 mg, 2.10 mmol, 1 eq) and 4-fluoro-5-(4,4,5,5-tetramethyl-1,3,2-dioxaborolan-2-yl)pyridin-2-amine (499 mg, 2.10 mmol, 1 eq) in dioxane (5 mL) and H₂O (1 mL) was added K₃PO₄ (667 mg, 3.14 mmol, 1.5 eq) and Pd(dppf)Cl₂·CH₂Cl₂ (171 mg, 210 mol, 0.1 eq) at 25° C. under N₂ atmosphere. The resulting mixture was stirred at 80° C. for 1 h under N₂ atmosphere. The reaction mixture was cooled down to room temperature and then concentrated under reduced pressure. The residue was purified by silica gel chromatography (Eluent of PE/EtOAc) to afford the title compound. LCMS: 280.0 [M+H]⁺

### Step 3. Synthesis of (1S,2R)-2-(5-(difluoromethyl)pyridin-2-yl)-N-(4′-fluoro-5-((S)-1-hydroxyethyl)-6-(methylthio)-[2,3′-bipyridin]-6′-yl)cyclopropane-1-carboxamide

To a solution of (1S)-1-[6-(6-amino-4-fluoro-3-pyridyl)-2-methylsulfanyl-3-pyridyl]ethanol (50 mg, 179 mol, 1 eq) in THF (1 mL) was added AlMe₃ (2 M in toluene, 5 eq) dropwise at 0° C. under N₂ atmosphere. The resulting mixture was stirred at 40° C. for 1 h. Then ethyl (1S,2R)-2-[5-(difluoromethyl)-2-pyridyl]cyclopropanecarboxylate (50 mg, 206 mol, 1.15 eq) in THF (1 mL) was added to the above solution at 25° C. under N₂ atmosphere. The resulting mixture was stirred at 90° C. for 15 hours. The reaction mixture was cooled to 0° C. and then quenched with sat. aq. potassium sodium tartrate solution (2 mL) at 0° C. under N₂. The resulting mixture was diluted with H₂O (8 mL) and then extracted with a mixture of DCM/MeOH (10/1 v/v, 10 mL*3). The combined organic layers were washed with brine (5 mL), dried over anhydrous Na₂SO₄, filtered and the filtrate was concentrated under reduced pressure. The residue was purified by silica gel chromatography (Eluent of PE/EtOAc) to afford the title compound. LCMS: 475.2 [M+H]⁺

### Step 4. Synthesis of (1S,2R)-2-[5-(difluoromethyl)pyridin-2-yl]-N-{5-[(1S)-1-hydroxyethyl]-4′-(methylamino)-6-(methylsulfanyl)-[2,3′-bipyridin]-6′-yl}cyclopropane-1-carboxamide

To a solution of (1S,2R)-2-[5-(difluoromethyl)-2-pyridyl]-N-[4-fluoro-5-[5-[(1S)-1-hydroxyethyl]-6-methylsulfanyl-2-pyridyl]-2-pyridyl]cyclopropanecarboxamide (19 mg, 40 mol, 1 eq) and methylamine hydrochloride (8 mg, 120 mol, 3 eq) in DMSO (1 mL) was added DIEA (31 mg, 240 mol, 6 eq). The reaction mixture was stirred at 100° C. for 16 h. The resulting mixture was cooled down to room temperature and then concentrated under reduced pressure. The residue was purified by reverse phase preparative HPLC (H₂O (NH₄HCO₃)-ACN) to afford the title compound. ¹H NMR (400 MHz, CD₃OD) δ=8.58 (d, 1H), 8.23 (s, 1H), 7.95-7.88 (m, 1H), 7.85 (d, 1H), 7.56 (d, 1H), 7.51 (d, 1H), 7.03-6.68 (m, 2H), 5.07 (q, 1H), 2.85 (s, 3H), 2.85-2.79 (m, 1H), 2.58 (s, 3H), 2.49 (d, 1H), 1.96 (d, 1H), 1.57 (d, 1H), 1.43 (d, 3H). LCMS: 486.2 [M+H]⁺

### Example 49. Synthesis of (1S,2R)-2-[5-(difluoromethyl)pyrimidin-2-yl]-N-{5-[(1S)-1-hydroxyethyl]-6-[3-(methylsulfanyl)-1-(2,2,2-trifluoroethyl)-1H-pyrazol-4-yl]-[2,3′-bipyridin]-6′-yl]cyclopropane-1-carboxamide (I-409)

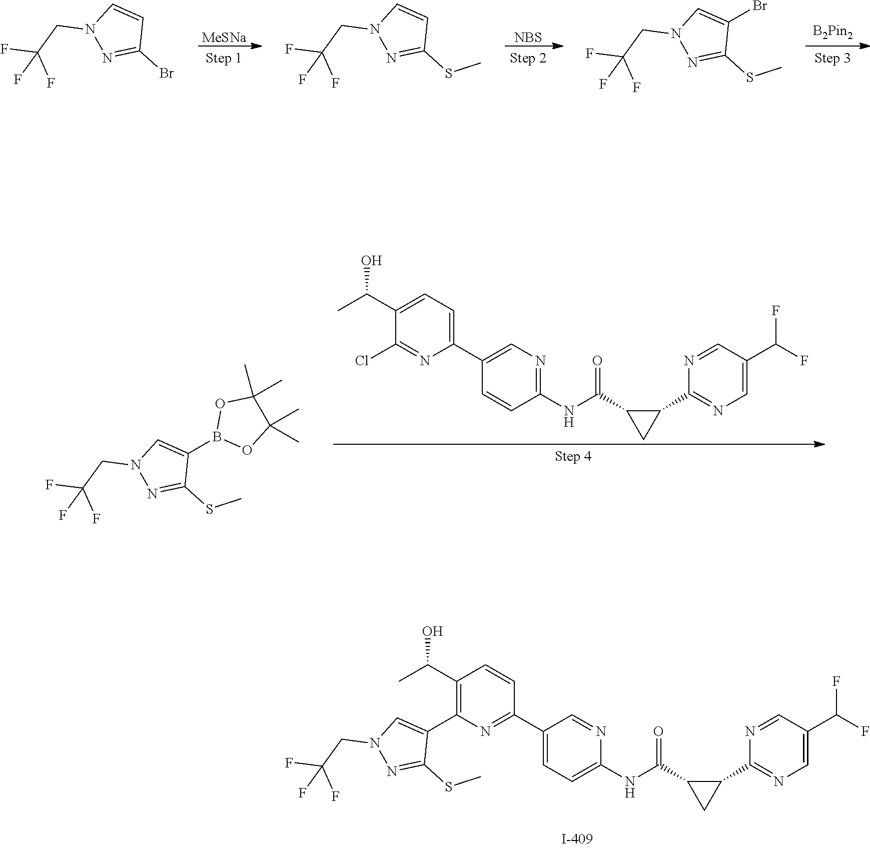

### Step 1. Synthesis of 3-(methylthio)-1-(2,2,2-trifluoroethyl)-1H-pyrazole

A mixture of 3-bromo-1-(2,2,2-trifluoroethyl)pyrazole (9.50 g, 41.49 mmol, 1 eq), sodium thiomethoxide (7.27 g, 103.71 mmol, 6.61 mL, 2.5 eq), DIEA (16.08 g, 124.46 mmol, 3 eq), Pd₂(dba)₃ (3.80 g, 4.15 mmol, 0.1 eq) and Xantphos (4.80 g, 8.30 mmol, 0.2 eq) in toluene (95 mL) was degassed and purged with N₂ for 3 times, and then the resulting mixture was stirred at 110° C. for 24 h under N₂ atmosphere. The reaction mixture was cooled down to room temperature, diluted with water (200 mL) and extracted with EtOAc (300 mL*3). The combined organic layers were washed with brine (200 mL), dried over anhydrous Na₂SO₄, filtered and the filtrate was concentrated under reduced pressure. The residue was purified by silica gel chromatography (Eluent of PE/EtOAc) to afford the title compound. LCMS: 197.1 [M+H]⁺

### Step 2. Synthesis of 4-bromo-3-(methylthio)-1-(2,2,2-trifluoroethyl)-1H-pyrazole

To a solution of 3-methylsulfanyl-1-(2,2,2-trifluoroethyl)pyrazole (1.25 g, 6.35 mmol, 1 eq) in ACN (12 mL) was added NBS (1.13 g, 6.35 mmol, 1 eq). The resulting mixture was stirred at 25° C. for 1 h. The reaction mixture was concentrated under reduced pressure and the residue was purified by silica gel chromatography (Eluent of PE/EtOAc) to afford the title compound. LCMS: 274.9 [M+H]⁺

### Step 3. Synthesis of 3-(methylthio)-4-(4,4,5,5-tetramethyl-1,3,2-dioxaborolan-2-yl)-1-(2,2,2-trifluoroethyl)-1H-pyrazole

A mixture of 4-bromo-3-methylsulfanyl-1-(2,2,2-trifluoroethyl)pyrazole (600 mg, 2.18 mmol, 1 eq), 4,4,5,5-tetramethyl-2-(4,4,5,5-tetramethyl-1,3,2-dioxaborolan-2-yl)-1,3,2-dioxaborolane (1.11 g, 4.36 mmol, 2 eq), KOAc (428 mg, 4.36 mmol, 2 eq) and PCy3-Pd-G3 (160 mg, 218 mol, 0.1 eq) in toluene (12 mL) was degassed and purged with N₂ for 3 times. The resulting mixture was stirred at 90° C. for 6 h under N₂ atmosphere. The reaction mixture was cooled down to room temperature, filtered and the filter cake was washed with EtOAc (100 mL). The combined filtrate was concentrated under reduced pressure. The residue was purified by silica gel chromatography (Eluent of PE/EtOAc) to afford the title compound. LCMS: 323.2 [M+H]⁺

### Step 4. Synthesis of (1S,2R)-2-[5-(difluoromethyl)pyrimidin-2-yl]-N-{5-[(1S)-1-hydroxyethyl]-6-[3-(methylsulfanyl)-1-(2,2,2-trifluoroethyl)-1H-pyrazol-4-yl]-[2,3′-bipyridin]-6′-yl}cyclopropane-1-carboxamide

A mixture of 3-methylsulfanyl-4-(4,4,5,5-tetramethyl-1,3,2-dioxaborolan-2-yl)-1-(2,2,2-trifluoroethyl)pyrazole (144 mg, 447 mol, 3 eq), (1S,2R)—N-[5-[6-chloro-5-[(1S)-1-hydroxyethyl]-2-pyridyl]-2-pyridyl]-2-[5-(difluoromethyl)pyrimidin-2-yl]cyclopropanecarboxamide (prepared as described in step 4 of I-150 synthesis) (66 mg, 149 μmol, 1 eq), Pd(dtbpf)Cl₂ (10 mg, 15 mol, 0.1 eq) and Et₃N (45 mg, 447 mol, 3 eq) in dioxane (2 mL) and H₂O (0.4 mL) was degassed and purged with N₂ for 3 times. The resulting mixture was stirred at 100° C. for 1 h under N₂ atmosphere. The reaction mixture was cooled down to room temperature and then concentrated under reduced pressure. The residue was purified by silica gel chromatography (Eluent of DCM/MeOH) followed by prep-HPLC (H₂O (0.05% NH₃·H₂O)-ACN) to afford the title compound. ¹H NMR (400 MHz, CD₃OD) δ=9.06 (d, 1H), 8.82 (s, 2H), 8.41 (dd, 1H), 8.10 (d, 1H), 8.07 (s, 1H), 7.92 (d, 1H), 7.82 (d, 1H), 6.92 (t, 1H), 5.08-4.95 (m, 3H), 3.00-2.88 (m, 1H), 2.63-2.53 (m, 1H), 2.50 (s, 3H), 2.09 (dt, 1H), 1.59 (dt, 1H), 1.47 (d, 3H). LCMS: 606.3 [M+H]⁺

### Example 50. Synthesis of (1S,2R)-2-[5-(difluoromethyl)pyridin-2-yl]-N-{15-[(1S)-1-hydroxyethyl]-13-oxa-4,8,18-triazatricyclo[12.3.1.0\hat{ }{2,7}]octadeca-1(17),2,4,6,14(18),15-hexaen-5-yl}cyclopropane-1-carboxamide (I-359

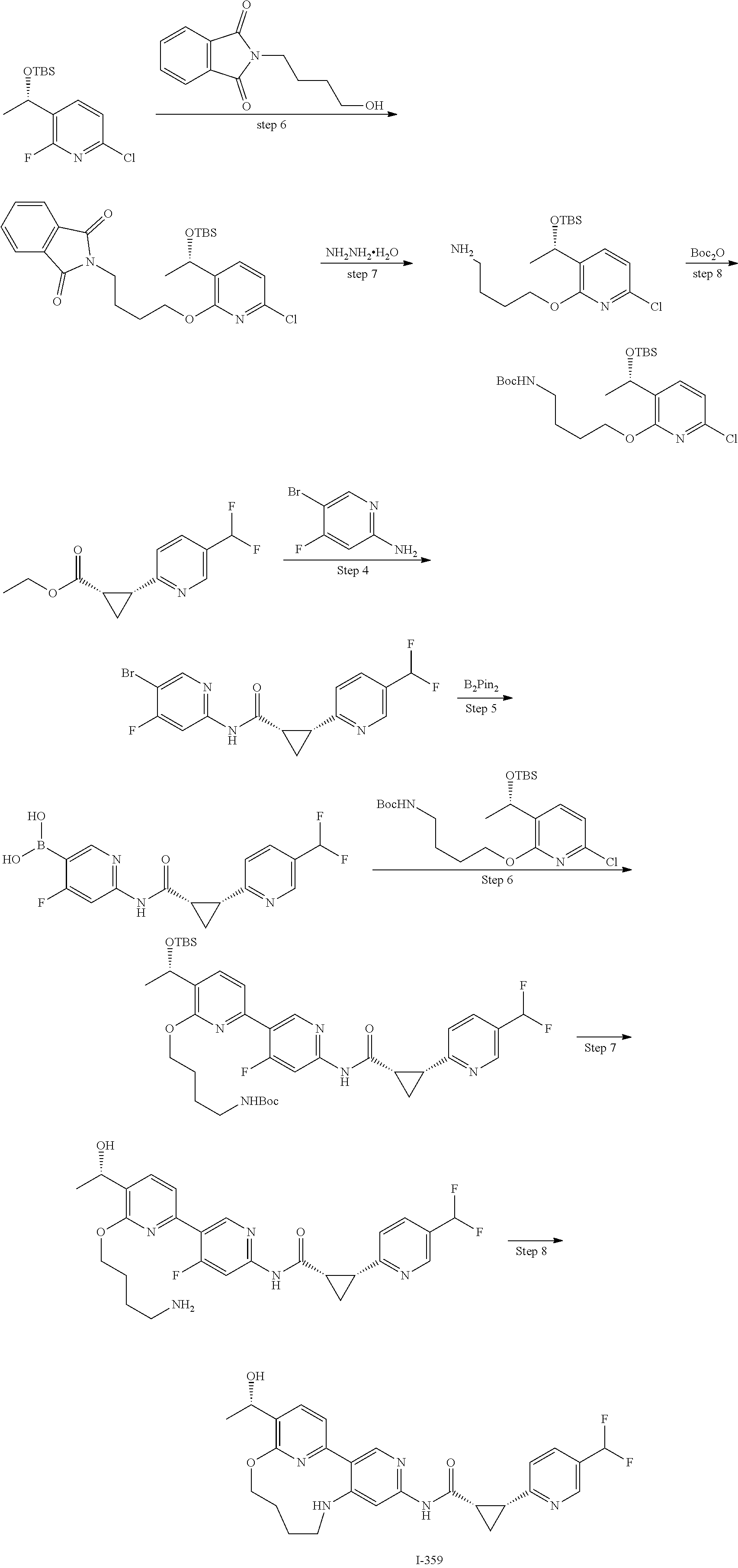

### Step 1. Synthesis of (S)-2-(4-((3-(1-((tert-butyldimethylsilyl)oxy)ethyl)-6-chloropyridin-2-yl)oxy)butyl)isoindoline-1,3-dione

To a solution of 2-(4-hydroxybutyl)isoindoline-1,3-dione (3.63 g, 16.56 mmol, 1.2 eq) in THF (40 mL) was added NaH (994 mg, 24.84 mmol, 60% in mineral oil, 1.8 eq) at 0° C. under N₂. The mixture was stirred at 0° C. under N₂ for 0.5 h. Then (S)-3-(1-((tert-butyldimethylsilyl)oxy)ethyl)-6-chloro-2-fluoropyridine (prepared from (S)-1-(6-chloro-2-fluoropyridin-3-yl)ethan-1-ol (step 1 of I-11) by analogy to step 2 of I-150 synthesis) (4.00 g, 13.80 mmol, 1.0 eq) was added and the resulting mixture was stirred at room temperature overnight under N₂ atmosphere. The reaction mixture was quenched with sat. aq. NH₄Cl (40 mL) and extracted with EtOAc (50 mL*2). The combined organic layers were washed with brine (40 mL), dried over anhydrous Na₂SO₄, filtered and the filtrate was concentrated under reduced pressure to afford the title compound, which was used in the next step without further purification. LCMS: 489.2 [M+H]⁺

### Step 2. Synthesis of (S)-4-((3-(1-((tert-butyldimethylsilyl)oxy)ethyl)-6-chloropyridin-2-yl)oxy)butan-1-amine

To a solution of (S)-2-(4-((3-(1-((tert-butyldimethylsilyl)oxy)ethyl)-6-chloropyridin-2-yl)oxy)butyl)isoindoline-1,3-dione (5.00 g, 10.22 mmol, 1.0 eq) in EtOH (20 mL) was added hydrazine hydrate (5.78 g, 115.46 mmol, 5.60 mL, 11.3 eq), and the resulting mixture was stirred at room temperature for 1 h. The reaction mixture was quenched with H₂O (30 mL) and then extracted with DCM (100 mL*2). The combined organic layers were washed with brine (60 mL), dried over anhydrous Na₂SO₄, filtered and the filtrate was concentrated under reduced pressure to afford the title compound, which was used in the next step without further purification. LCMS: 359.0 [M+H]⁺

### Step 3. Synthesis of tert-butyl (S)-(4-((3-(1-((tert-butyldimethylsilyl)oxy)ethyl)-6-chloropyridin-2-yl)oxy)butyl)carbamate

To a solution of (S)-4-((3-(1-((tert-butyldimethylsilyl)oxy)ethyl)-6-chloropyridin-2-yl)oxy)butan-1-amine (3.67 g, 10.22 mmol, 1.0 eq) in DCM (40 mL) was added DIEA (2.64 g, 20.45 mmol, 2.0 eq) and Boc₂O (3.35 g, 15.34 mmol, 1.5 eq) and the resulting mixture was stirred at room temperature for 0.5 h. The reaction mixture was diluted with H₂O (50 mL) and then extracted with DCM (50 mL*2). The combined organic layers were washed with brine (30 mL), dried over anhydrous Na₂SO₄, filtered and the filtrate was concentrated under reduced pressure. The residue was purified by silica gel chromatography (Eluent of EtOAc/PE) to afford the tittle compound. LCMS: 459.1 [M+H]⁺

### Step 4. Synthesis of (1S,2R)—N-(5-bromo-4-fluoropyridin-2-yl)-2-(5-(difluoromethyl)pyridin-2-yl)cyclopropane-1-carboxamide

To a solution of 5-bromo-4-fluoropyridin-2-amine (4.00 g, 20.94 mmol, 1.0 eq) and ethyl (1S,2R)-2-(5-(difluoromethyl)pyridin-2-yl)cyclopropane-1-carboxylate (6.06 g, 25.13 mmol, 1.2 eq) in THF (40 mL) was added LiHMDS (1 M in THF, 27.23 mL, 1.3 eq) at 0° C., and the resulting mixture was stirred at room temperature for 0.5 h under N₂ atmosphere. The reaction mixture was quenched with sat. aq. NH₄Cl (10 mL) and H₂O (50 mL) and the resulting mixture was extracted with EtOAc (50 mL*2). The combined organic layers were washed with brine (50 mL), dried over anhydrous Na₂SO₄, filtered and the filtrate was concentrated under reduced pressure. The residue was purified by silica gel chromatography (Eluent of EtOAc/PE) to afford the tittle compound. LCMS: 388.1 [M+H]⁺

### Step 5. Synthesis of (6-((1S,2R)-2-(5-(difluoromethyl)pyridin-2-yl)cyclopropane-1-carboxamido)-4-fluoropyridin-3-yl)boronic acid

To a solution of (1S,2R)—N-(5-bromo-4-fluoropyridin-2-yl)-2-(5-(difluoromethyl)pyridin-2-yl)cyclopropane-1-carboxamide (2.65 g, 6.86 mmol, 1.0 eq) and 4,4,4′,4′,5,5,5′,5′-octamethyl-2,2′-bi(1,3,2-dioxaborolane) (1.92 g, 7.55 mmol, 1.1 eq) in 1,4-dioxane (30 mL) was added KOAc (1.35 g, 13.72 mmol, 2.0 eq) and Pd(dppf)Cl₂ (502 mg, 686 mol, 0.1 eq) and the resulting mixture was stirred at 90° C. for 4 h under N₂ atmosphere. The reaction mixture was filtered and the filtrate was concentrated under reduced pressure to afford the title compound, which was used in the next step without further purification. LCMS: 352.0 [M+H]⁺

### Step 6. Synthesis of tert-butyl (4-((5-((S)-1-((tert-butyldimethylsilyl)oxy)ethyl)-6′-((1S,2R)-2-(5-(difluoromethyl)pyridin-2-yl)cyclopropane-1-carboxamido)-4′-fluoro-[2,3′-bipyridin]-6-yl)oxy)butyl)carbamate

To a solution of tert-butyl (S)-(4-((3-(1-((tert-butyldimethylsilyl)oxy)ethyl)-6-chloropyridin-2-yl)oxy)butyl)carbamate (from step 3, 2.00 g, 4.36 mmol, 1.0 eq) and (6-((1S,2R)-2-(5-(difluoromethyl)pyridin-2-yl)cyclopropane-1-carboxamido)-4-fluoropyridin-3-yl)boronic acid (2.83 g, 8.06 mmol, 1.8 eq) in 1,4-dioxane (40 mL) and H₂O (8 mL) was added K₃PO₄ (1.85 g, 8.71 mmol, 2.0 eq) and Pd(dppf)C12 (319 mg, 436 μmol, 0.1 eq), and the resulting mixture was stirred at 60° C. overnight under N₂ atmosphere. The reaction mixture was diluted with H₂O (100 mL) and extracted with EtOAc (100 mL*2). The combined organic layers were washed with brine (40 mL), dried over anhydrous Na₂SO₄, filtered and the filtrate was concentrated under reduced pressure. The residue was purified by silica gel chromatography (Eluent of EtOAc/PE) to afford the tittle compound. LCMS: 730.1 [M+H]⁺

### Step 7. Synthesis of (1S,2R)—N-(6-(4-aminobutoxy)-4′-fluoro-5-((S)-1-hydroxyethyl)-[2,3′-bipyridin]-6′-yl)-2-(5-(difluoromethyl)pyridin-2-yl)cyclopropane-1-carboxamide

To tert-butyl (4-((5-((S)-1-((tert-butyldimethylsilyl)oxy)ethyl)-6′-((1S,2R)-2-(5-(difluoromethyl)pyridin-2-yl)cyclopropane-1-carboxamido)-4′-fluoro-[2,3′-bipyridin]-6-yl)oxy)butyl)carbamate (750 mg, 1.03 mmol, 1.0 eq) was added a solution of HCl in 1,4-dioxane (2 M, 5 mL), and the resulting mixture was stirred at room temperature for 1 hour. The reaction mixture was concentrated under reduced pressure. The residue was purified by reverse phase HPLC (C18 column, water (0.1% NH₄HCO₃)-ACN) to afford the title compound. LCMS:516.1 [M+H]⁺

### Step 8. Synthesis of (1S,2R)-2-[5-(difluoromethyl)pyridin-2-yl]-N-{15-[(1S)-1-hydroxyethyl]-13-oxa-4,8,18-triazatricyclo[12.3.1.0\hat{ }{2,7}]octadeca-1(17),2,4,6,14(18),15-hexaen-5-yl}cyclopropane-1-carboxamide

The solution of (1S,2R)—N-(6-(4-aminobutoxy)-4′-fluoro-5-((S)-1-hydroxyethyl)-[2,3′-bipyridin]-6′-yl)-2-(5-(difluoromethyl)pyridin-2-yl)cyclopropane-1-carboxamide (100 mg, 194 mol, 1.0 eq) in DMSO (15 mL) was stirred at 140° C. for 1 h. The reaction mixture was diluted with H₂O (30 mL) and then extracted with EtOAc (10 mL*2). The combined organic layers were washed with brine (40 mL), dried over anhydrous Na₂SO₄, filtered and the filtrate was concentrated under reduced pressure. The residue was purified first by silica gel chromatography (Eluent of MeOH/DCM, 0.1% Et₃N) and then further purified by reverse phase HPLC (C18 column, water (0.1% NH₄HCO₃)-ACN) to afford the tittle compound. ¹H NMR (400 MHz, CDCl₃) δ 10.18 (br s, 1H), 8.86 (br s, 1H), 8.66 (s, 1H), 8.45 (s, 1H), 7.82-7.75 (m, 1H), 7.73 (d, 1H), 7.47-7.42 (m, 2H), 7.33 (d, 1H), 6.88-6.51 (m, 1H), 5.12-4.96 (m, 1H), 4.44 (t, 2H), 3.28 (br s, 2H), 2.86-2.70 (m, 1H), 2.36-2.24 (m, 1H), 2.11-2.01 (m, 1H), 1.93 (br d, 2H), 1.87-1.80 (m, 2H), 1.62-1.57 (m, 1H), 1.53 (d, 3H). LCMS:496.1 [M+H]⁺.

### Example 51. Synthesis of (1S,2R)-2-[5-(difluoromethyl)pyridin-2-yl]-N-{20-[(1S)-1-hydroxyethyl]-4-(2,2,2-trifluoroethyl)-4,5,10,14,21-pentaazatetracyclo[15.3.1.0\hat{ }{2,6}.0\hat{ }{11,16}]henicosa-1(21),2,5,11,13,15,17,19-octaen-13-yl}cyclopropane-1-carboxamide (I-338)

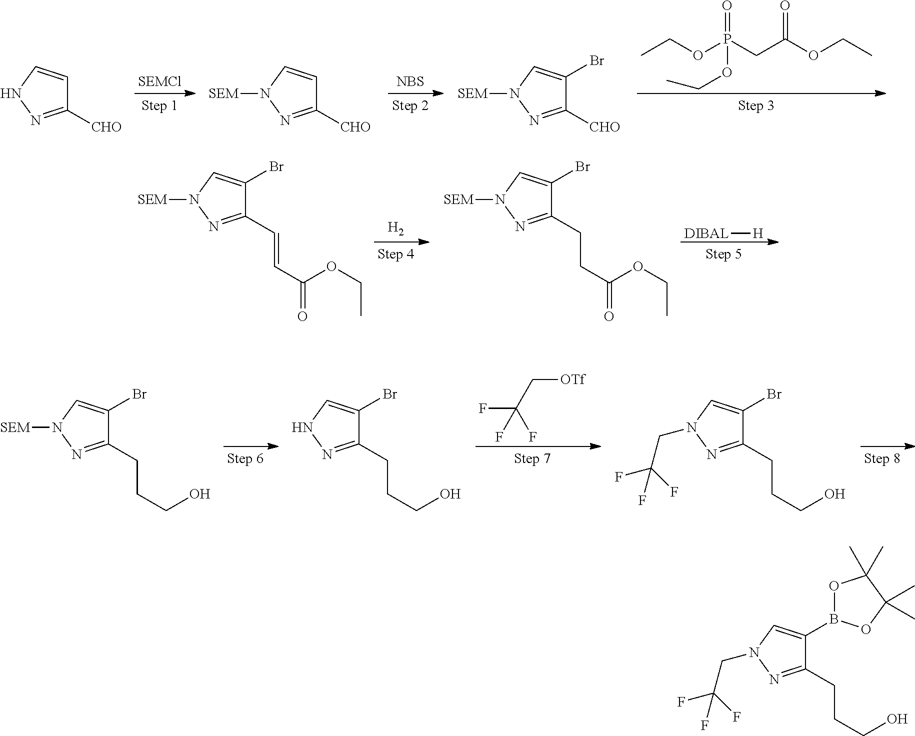

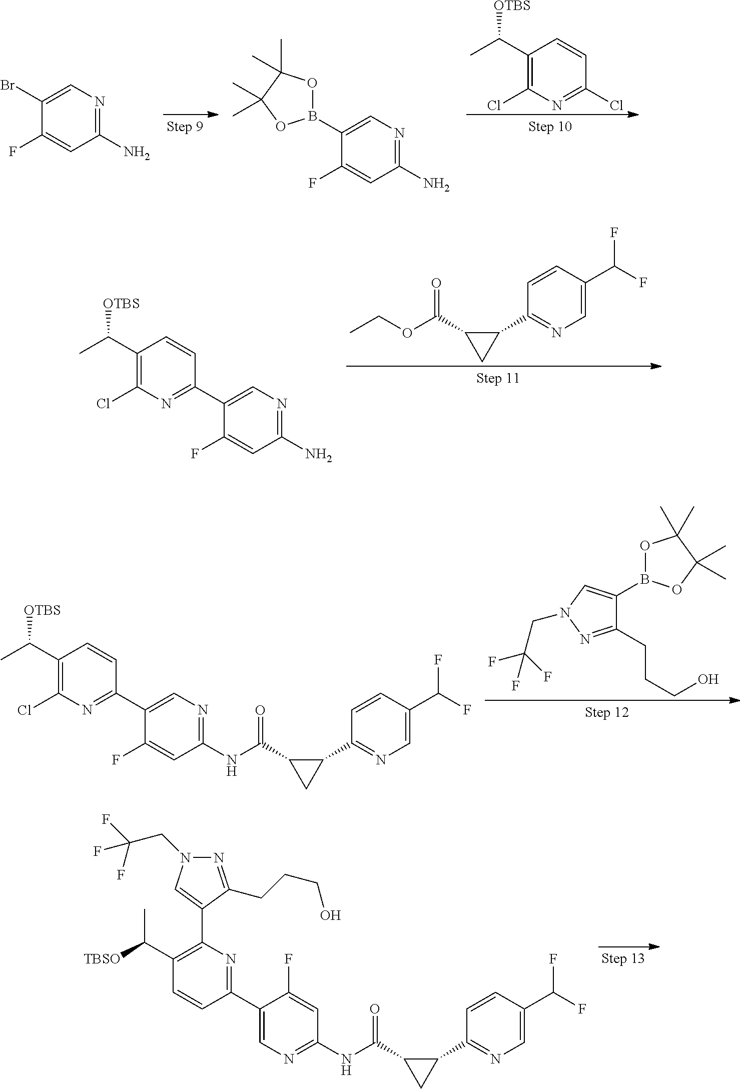

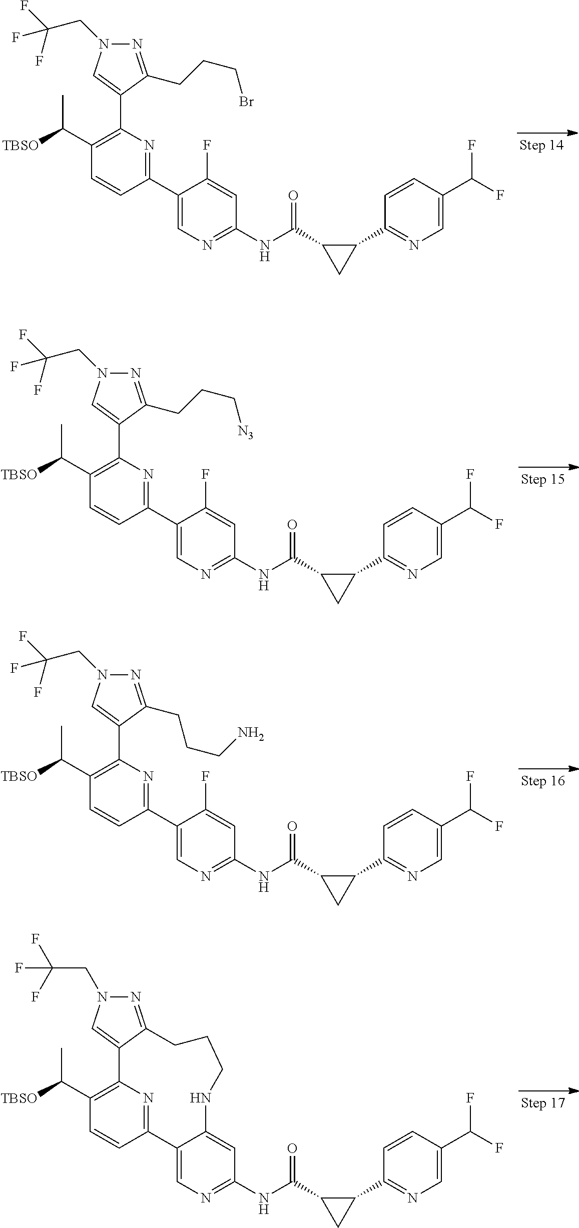 [SMILES: C[C@H](O[Si](C)(C)C(C)(C)C)c1ccc(-c2cnc(NC(=O)[C@H]3C[C@H]3c3ccc(C(F)F)cn3)cc2F)nc1-c1cn(CC(F)(F)F)nc1CCCBr ; C[C@H](O[Si](C)(C)C(C)(C)C)c1ccc(-c2cnc(NC(=O)[C@H]3C[C@H]3c3ccc(C(F)F)cn3)cc2F)nc1-c1cn(CC(F)(F)F)nc1CCCN=[N+]=[N-] ; C[C@H](O[Si](C)(C)C(C)(C)C)c1ccc(-c2cnc(NC(=O)[C@H]3C[C@H]3c3ccc(C(F)F)cn3)cc2F)nc1-c1cn(CC(F)(F)F)nc1CCCN ; C[C@H](O[Si](C)(C)C(C)(C)C)c1ccc2nc1-c1cn(CC(F)(F)F)nc1CCCNc1cc(NC(=O)[C@H]3C[C@H]3c3ccc(C(F)F)cn3)ncc1-2]

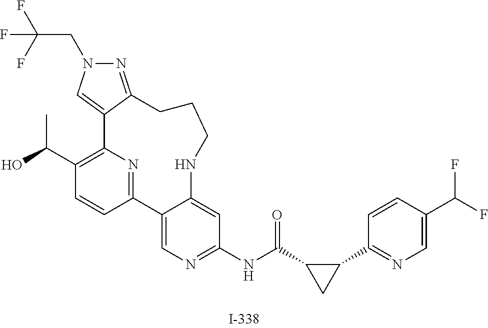 [SMILES: C[C@H](O)c1ccc2nc1-c1cn(CC(F)(F)F)nc1CCCNc1cc(NC(=O)[C@H]3C[C@H]3c3ccc(C(F)F)cn3)ncc1-2]

### Step 1. Synthesis of 1-((2-(trimethylsilyl)ethoxy)methyl)-1H-pyrazole-3-carbaldehyde

To a solution of 1H-pyrazole-3-carbaldehyde (20.00 g, 208.14 mmol, 1 eq) and DIEA (53.80 g, 416.29 mmol, 2 eq) in DCM (200 mL) was added SEM-C1 (52.05 g, 312.22 mmol, 1.5 eq) at 0° C. and the resulting mixture was stirred at 25° C. for 16 h. The reaction mixture was concentrated under reduced pressure and the residue was purified by silica gel chromatography (Eluent of PE/EtOAc) to afford the title compound. ¹H NMR (400 MHz, CDCl₃) δ=10.02 (s, 1H), 7.64 (d, 1H), 6.88 (d, 1H), 5.52 (s, 2H), 3.62-3.57 (m, 2H), 0.96-0.90 (m, 2H), −0.01 (s, 9H).

### Step 2. Synthesis of 4-bromo-1-((2-(trimethylsilyl)ethoxy)methyl)-1H-pyrazole-3-carbaldehyde

To a solution of 1-((2-(trimethylsilyl)ethoxy)methyl)-1H-pyrazole-3-carbaldehyde (30.90 g, 136.52 mmol, 1 eq) in ACN (310 mL) was added NBS (26.73 g, 150.17 mmol, 1.1 eq) and the resulting mixture was stirred at 25° C. for 16 h. The reaction mixture was concentrated under reduced pressure and the residue was purified by silica gel chromatography (Eluent of PE/EtOAc) to afford the title compound. ¹H NMR (400 MHz, CDCl₃) δ=10.00 (s, 1H), 7.71 (s, 1H), 5.48 (s, 2H), 3.62-3.57 (m, 2H), 0.92 (d, 2H), 0.00 (s, 9H).

### Step 3. Synthesis of ethyl (E)-3-(4-bromo-1-((2-(trimethylsilyl)ethoxy)methyl)-1H-pyrazol-3-yl)acrylate

To a suspension of NaH (3.93 g, 98.28 mmol, 60% purity, 1.2 eq) in THF (250 mL) was added ethyl 2-diethoxyphosphorylacetate (22.03 g, 98.28 mmol, 1.2 eq) at 0° C. and the resulting mixture was stirred at 0° C. for 0.5 hour. Then a solution of 4-bromo-1-((2-(trimethylsilyl)ethoxy)methyl)-1H-pyrazole-3-carbaldehyde (25.00 g, 81.90 mmol, 1 eq) in THF (250 mL) was added and the reaction mixture was stirred at 25° C. for 2 hours. The reaction mixture was quenched with sat. aq. NH₄Cl solution (100 mL), poured into water (1000 mL) and extracted with EtOAc (3*300 mL). The combined organic phase was washed with brine (3*300 mL), dried over anhydrous Na₂SO₄, filtered and the filtrate was concentrated under reduced pressure. The residue was purified by flash silica gel chromatography (Eluent of PE/EtOAc) to afford the title compound. LCMS: 375.2 [M+H]⁺

### Step 4. Synthesis of ethyl 3-(4-bromo-1-((2-(trimethylsilyl)ethoxy)methyl)-1H-pyrazol-3-yl)propanoate

This step was performed using flow chemistry. Solution 1: ethyl (E)-3-(4-bromo-1-((2-(trimethylsilyl)ethoxy)methyl)-1H-pyrazol-3-yl)acrylate (17.00 g, 1.0 eq) was dissolved in 200 mL THF. The solution 1 was delivered by a plunger pump at a flow rate of 1.20 mL/min. H₂ (from a high-pressure cylinder) was introduced via a mass flow controller (MFC) at 60 sccm (11 equiv) and the system was maintained at 1 MPa. The liquid and gas streams were mixed with a T-shaped mixer and the combined solution was passed into a stainless-steel fixed-bed reactor (outer diameter: 6.350 mm (½ inch); internal volume: 5.00 mL) packed with 20 g of WXSC1025 (5% Ru/Al₂O₃nanoparticle immobilized catalyst obtained from WuXi AppTech) and held at 60° C. Upon complete injection of the reaction solution, the solvent feed was switched to THF at 1.2 mL/min and the reactor was flushed for 120 min. The resulting mixture was concentrated under reduced pressure. The residue was purified by flash silica gel chromatography (Eluent of PE/EtOAc) to afford the title compound. LCMS: 377.2 [M+H]⁺

### Step 5. Synthesis of 3-(4-bromo-1-((2-(trimethylsilyl)ethoxy)methyl)-1H-pyrazol-3-yl)propan-1-ol

This step was performed using flow chemistry. Solution 1: ethyl 3-(4-bromo-1-((2-(trimethylsilyl)ethoxy)methyl)-1H-pyrazol-3-yl)propanoate (12.80 g, 1.0 eq) was dissolved in 128 mL DCM. Solution 2: DIBAL-H (87.13 g, 1.0 M, 102.51 mL, 3.0 equiv). Solution 1 and 2 were mixed with a T-shaped mixer at 25.0° C. with flow rates of 6.13 mL/min and 4.56 mL/min, respectively. The combined solution was passed into a perfluoroalkoxy (PFA) Coils reactor (outer diameter: 3.175 mm (⅛ inch); internal volume: 21.37 mL) maintained at 25.0° C., and then into stainless-steel dynamic mixer (outer diameter: 4.763 mm( 3/16 inch); internal volume: 0.93 mL) operated at 25.0° C. The residence time of flow reactor was 2.00 min. The resulting mixture was poured into sat. aq. potassium sodium tartrate solution (500 mL) and extracted with EtOAc (3*200 mL). The combined organic phase was washed with brine (3*200 mL), dried over anhydrous Na₂SO₄, filtered and the filtrate was concentrated under reduced pressure. The residue was purified by flash silica gel chromatography (Eluent of PE/EtOAc) to afford the title compound. LCMS: 335.3 [M+H]⁺

### Step 6. Synthesis of 3-(4-bromo-1H-pyrazol-3-yl)propan-1-ol

To a solution of 3-[4-bromo-1-(2-trimethylsilylethoxymethyl)pyrazol-3-yl]propan-1-ol (2.70 g, 8.05 mmol, 1 eq) in THF (7 mL) was added TBAF (1 M in THF, 20.13 mL, 2.5 eq, in THF). The resulting mixture was stirred at 80° C. for 6 h. The reaction mixture was filtered and the filtrate was concentrated under reduced pressure to afford the title compound which was used in the next step directly.

### Step 7. Synthesis of 3-(4-bromo-1-(2,2,2-trifluoroethyl)-1H-pyrazol-3-yl)propan-1-ol

To a solution of 3-(4-bromo-1H-pyrazol-3-yl)propan-1-ol (1.65 g, 8.05 mmol, 1 eq) and Cs₂CO₃ (7.87 g, 24.14 mmol, 3 eq) in DMF (16 mL) was added 2,2,2-trifluoroethyl trifluoromethanesulfonate (3.74 g, 16.09 mmol, 2 eq) at 0° C. The resulting mixture was stirred at 0° C. for 1 h. The reaction mixture was diluted with water (30 mL) and extracted with EtOAc (50 mL*3). The combined organic layers were washed with brine (20 mL*3), dried over anhydrous Na₂SO₄, filtered and the filtrate was concentrated under reduced pressure. The residue was purified by flash silica gel chromatography (Eluent of PE/EtOAc) to afford the title compound. LCMS: 287.0 [M+H]⁺

### Step 8. Synthesis of 3-(4-(4,4,5,5-tetramethyl-1,3,2-dioxaborolan-2-yl)-1-(2,2,2-trifluoroethyl)-1H-pyrazol-3-yl)propan-1-ol

A mixture of 3-(4-bromo-1-(2,2,2-trifluoroethyl)-1H-pyrazol-3-yl)propan-1-ol (1.86 g, 6.49 mmol, 1 eq), 4,4,5,5-tetramethyl-2-(4,4,5,5-tetramethyl-1,3,2-dioxaborolan-2-yl)-1,3,2-dioxaborolane (1.98 g, 7.79 mmol, 1.2 eq), PCy3-Pd-G3 (477 mg, 649 mol, 0.1 eq) and KOAc (1.27 g, 12.99 mmol, 2 eq) in toluene (18 mL) was degassed and purged with N₂ for 3 times. The resulting mixture was stirred at 100° C. for 1 h under N₂ atmosphere. The reaction mixture was cooled to room temperature, filtered and the filter cake was washed with EtOAc (30 mL). The combined filtrate was concentrated under reduced pressure. The residue was purified by flash silica gel chromatography (Eluent of PE/EtOAc) to afford the title compound. LCMS: 335.2 [M+H]⁺

### Step 9. Synthesis of 4-fluoro-5-(4,4,5,5-tetramethyl-1,3,2-dioxaborolan-2-yl)pyridin-2-amine

A mixture of 5-bromo-4-fluoro-pyridin-2-amine (20.00 g, 104.71 mmol, 1 eq), 4,4,5,5-tetramethyl-2-(4,4,5,5-tetramethyl-1,3,2-dioxaborolan-2-yl)-1,3,2-dioxaborolane (27.92 g, 109.95 mmol, 1.05 eq), Pd(dppf)Cl₂·CH₂Cl₂ (4.28 g, 5.24 mmol, 0.05 eq) and KOAc (20.55 g, 209.42 mmol, 2 eq) in dioxane (200 mL) was stirred at 110° C. for 2 h. The reaction mixture was filtered and the filtrate was concentrated under reduced pressure. The residue was purified by flash silica gel chromatography (Eluent of PE/EtOAc) to afford the title compound. LCMS: 239.0 [M+H]⁺

### Step 10. Synthesis of (S)-5-(1-((tert-butyldimethylsilyl)oxy)ethyl)-6-chloro-4′-fluoro-[2,3′-bipyridin]-6′-amine

A mixture of 4-fluoro-5-(4,4,5,5-tetramethyl-1,3,2-dioxaborolan-2-yl)pyridin-2-amine (11.50 g, 48.31 mmol, 1 eq), (S)-3-(1-((tert-butyldimethylsilyl)oxy)ethyl)-2,6-dichloropyridine (obtained from (S)-1-(2,6-dichloropyridin-3-yl)ethan-1-ol (step 1 of I-100) by analogy to step 2 of I-150 synthesis) (14.80 g, 48.31 mmol, 1 eq), Pd(dppf)Cl₂·CH₂Cl₂ (1.97 g, 2.42 mmol, 0.05 eq) and K₃PO₄ (25.63 g, 120.76 mmol, 2.5 eq) in dioxane (115 mL) and H₂O (23 mL) was stirred at 40° C. for 2 hours. The reaction mixture was filtered and the filtrate was concentrated under reduced pressure. The residue was purified by flash silica gel chromatography (Eluent of PE/EtOAc) to afford the title compound. LCMS: 382.2 [M+H]⁺

### Step 11. Synthesis of (1S,2R)—N-(5-((S)-1-((tert-butyldimethylsilyl)oxy)ethyl)-6-chloro-4′-fluoro-[2,3′-bipyridin]-6′-yl)-2-(5-(difluoromethyl)pyridin-2-yl)cyclopropane-1-carboxamide

To a solution of (S)-5-(1-((tert-butyldimethylsilyl)oxy)ethyl)-6-chloro-4′-fluoro-[2,3′-bipyridin]-6′-amine (3.00 g, 7.85 mmol, 1 eq) and ethyl (1S,2R)-2-(5-(difluoromethyl)pyridin-2-yl)cyclopropane-1-carboxylate (1.89 g, 7.85 mmol, 1 eq) in toluene (30 mL) was added LiHMDS (1 M in THF, 15.71 mL, 2 eq) at 25° C. The resulting mixture was stirred at 40° C. for 1 h, then poured into sat. aq. NH₄Cl solution (200 mL) and extracted with EtOAc (3*100 mL). The combined organic phase was washed with brine (200 mL), dried over anhydrous Na₂SO₄, filtered and the filtrate was concentrated under reduced pressure. The residue was purified by flash silica gel chromatography (Eluent of PE/EtOAc) to afford the title compound. LCMS: 577.3 [M+H]⁺

### Step 12. Synthesis of (1S,2R)—N-(5-((S)-1-((tert-butyldimethylsilyl)oxy)ethyl)-4′-fluoro-6-(3-(3-hydroxypropyl)-1-(2,2,2-trifluoroethyl)-1H-pyrazol-4-yl)-[2,3′-bipyridin]-6′-yl)-2-(5-(difluoromethyl)pyridin-2-yl)cyclopropane-1-carboxamide

A mixture of (1S,2R)—N-(5-((S)-1-((tert-butyldimethylsilyl)oxy)ethyl)-6-chloro-4′-fluoro-[2,3′-bipyridin]-6′-yl)-2-(5-(difluoromethyl)pyridin-2-yl)cyclopropane-1-carboxamide (474 mg, 821 mol, 1 eq), 3-(4-(4,4,5,5-tetramethyl-1,3,2-dioxaborolan-2-yl)-1-(2,2,2-trifluoroethyl)-1H-pyrazol-3-yl)propan-1-ol (823 mg, 2.46 mmol, 3 eq), Pd(dtbpf)Cl₂ (54 mg, 82 mol, 0.1 eq) and Et₃N (249 mg, 2.46 mmol, 3 eq) in dioxane (10 mL) and H₂O (2 mL) was stirred at 100° C. for 1 hour. The reaction mixture was cooled down to room temperature and concentrated under reduced pressure. The residue was purified by flash silica gel chromatography (Eluent of PE/EtOAc followed by EtOAc/MeOH) to afford the title compound. LCMS: 749.5 [M+H]⁺

### Step 13. Synthesis of (1S,2R)—N-(6-(3-(3-bromopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide

To a solution of (1S,2R)—N-(5-((S)-1-((tert-butyldimethylsilyl)oxy)ethyl)-4′-fluoro-6-(3-(3-hydroxypropyl)-1-(2,2,2-trifluoroethyl)-1H-pyrazol-4-yl)-[2,3′-bipyridin]-6′-yl)-2-(5-(difluoromethyl)pyridin-2-yl)cyclopropane-1-carboxamide (558 mg, 745 mol, 1 eq) and PPh₃ (293 mg, 1.12 mmol, 1.5 eq) in DCM (5.5 mL) was added CBr₄ (371 mg, 1.12 mmol, 1.5 eq) at 0° C. and the resulting mixture was stirred at 25° C. for 2 hours. The reaction mixture was concentrated under reduced pressure. The residue was purified by flash silica gel chromatography (Eluent of PE/EtOAc) to afford the title compound. LCMS: 811.4 [M+H]⁺

### Step 14. Synthesis of (1S,2R)—N-(6-(3-(3-azidopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide

To a solution of (1S,2R)—N-(6-(3-(3-bromopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide (611 mg, 752 mol, 1 eq) in DMF (6 mL) was added NaN₃ (59 mg, 903 mol, 1.2 eq), then the resulting mixture was stirred at 60° C. for 2 h. The reaction mixture was diluted with water (50 mL) and extracted with EtOAc (30 mL*3). The combined organic layers were washed with brine (40 mL*3), dried over anhydrous sodium sulfate, filtered and the filtrate was concentrated under reduced pressure to afford the title compound, which was used in the next step directly. LCMS: 774.4 [M+H]⁺

### Step 15. Synthesis of (1S,2R)—N-(6-(3-(3-aminopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide

To a solution of (1S,2R)—N-(6-(3-(3-azidopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide (580 mg, 749 mol, 1 eq) in THF (6 mL) and H₂O (0.6 mL) was added PPh₃ (295 mg, 1.12 mmol, 1.5 eq), the resulting mixture was stirred at 50° C. for 2 hours. The reaction mixture was concentrated under reduced pressure. The residue was purified by reversed phase preparative HPLC (H₂O (0.1% FA)/ACN) to afford the title compound. LCMS: 748.4 [M+H]⁺

### Step 16. Synthesis of (1S,2R)—N-(5-((S)-1-((tert-butyldimethylsilyl)oxy)ethyl)-2-(2,2,2-trifluoroethyl)-13,14,15,16-tetrahydro-2H-4,8-(azeno)pyrazolo[4,3-i]pyrido[4,3-b][1]azacyclotridecin-11-yl)-2-(5-(difluoromethyl)pyridin-2-yl)cyclopropane-1-carboxamide

The solution of (1S,2R)—N-(6-(3-(3-aminopropyl)-1-(2,2,2-trifluoroethyl)-1H-pyrazol-4-yl)-5-((S)-1-((tert-butyldimethylsilyl)oxy)ethyl)-4′-fluoro-[2,3′-bipyridin]-6′-yl)-2-(5-(difluoromethyl)pyridin-2-yl)cyclopropane-1-carboxamide (300 mg, 401 mol, 1 eq) and DIEA (104 mg, 802 mol, 2 eq) in DMSO (3 mL) was stirred at 100° C. for 2 hours. The resulting mixture was poured into water (40 mL) and extracted with EtOA (3×20 mL). The combined organic phase was washed with brine (3*20 mL), dried over anhydrous Na₂SO₄, filtered and the filtrate was concentrated under reduced pressure to afford the title compound. LCMS: 728.6 [M+H]⁺

### Step 17. Synthesis of (1S,2R)-2-[5-(difluoromethyl)pyridin-2-yl]-N-{20-[(1S)-1-hydroxyethyl]-4-(2,2,2-trifluoroethyl)-4,5,10,14,21-pentaazatetracyclo[15.3.1.0\hat{ }{2,6}.0\hat{ }{11,16}]henicosa-1(21),2,5,11,13,15,17,19-octaen-13-yl}cyclopropane-1-carboxamide

To a solution of (1S,2R)—N-(5-((S)-1-((tert-butyldimethylsilyl)oxy)ethyl)-2-(2,2,2-trifluoroethyl)-13,14,15,16-tetrahydro-2H-4,8-(azeno)pyrazolo[4,3-i]pyrido[4,3-b][1]azacyclotridecin-11-yl)-2-(5-(difluoromethyl)pyridin-2-yl)cyclopropane-1-carboxamide (290 mg, 398 mol, 1 eq) in THF (3 mL) was added TBAF (1 M, 597.66 μL, 1.5 eq, in THF) and the resulting mixture was stirred at 25° C. for 1 hour. The reaction mixture was concentrated under reduced pressure. The residue was purified by flash silica gel chromatography (Eluent of PE/EtOAc and EtOAc/MeOH), prep-HPLC (H₂O (0.05% NH₃·H₂O)-ACN) and SFC (SFC Preparative Method: Instrument: Waters 150 Preparative SFC system; Column: DAICEL CHIRALPAK® AD 250×40 mm I.D., 10 m; Mobile phase A: supercritical CO₂, Mobile Phase B: IPA:ACN 4:1 (0.1% NH₃*H₂O); Isocratic elution: 55% Phase B in Supercritical CO₂; Flow rate: 120 g/min; Retention time: 3.72 min. Back Pressure: 100 bar to keep the CO₂ in Supercritical flow; Wave Length: 220 nm) to afford the title compound. ¹H NMR (400 MHz, CD₃OD) δ=8.58 (s, 1H), 8.47 (s, 1H), 8.13-8.07 (m, 2H), 7.90 (d, 1H), 7.82 (d, 1H), 7.54 (d, 1H), 7.16 (s, 1H), 7.00-6.70 (m, 1H), 5.13-5.09 (m, 1H), 4.99-4.95 (m, 2H), 3.23-3.18 (m, 2H), 2.91-2.84 (m, 2H), 2.83-2.75 (m, 1H), 2.54-2.45 (m, 1H), 2.20-2.10 (m, 2H), 1.98-1.91 (m, 1H), 1.58-1.50 (m, 4H). LCMS: 614.3 [M+H]⁺. Analytical chiral SFC R^{†}: 0.911 min.

### Analytical Chiral SFC Method:

| Instrument | SHIMADZU LC-30ADsf | |
|---|---|---|
| Column | CHIRALPAK ® AD-3 50 × 4.6 mm I.D., particle | |
| | size 3 μm | |
| Mobile Phase | Phase A for CO₂ | Phase B for IPA + ACN 2:1 |
| | | (0.05% diethylamine) |

| Gradient | Time(min) | A(%) | B(%) |
|---|---|---|---|
| | 0.00 | 70 | 30 |
| | 1.20 | 40 | 60 |
| | 1.70 | 40 | 60 |
| | 1.71 | 70 | 30 |
| | 2.00 | 70 | 30 |

| | |
|---|---|
| Flow rate | 4.0 mL/min |
| Column Temperature | 35° C. |
| Back pressure | 100 Bar |
| UV wave length | 220 nm |

### Example 52. Synthesis of (1S,2R)—N-(6-{1-[(1R*)-2,2-difluorocyclobutyl]-3-methyl-1H-pyrazol-4-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 1) (I-389) and (1S,2R)—N-(6-{1-[(1R*)-2,2-difluorocyclobutyl]-3-methyl-1H-pyrazol-4-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 2) (I-394)

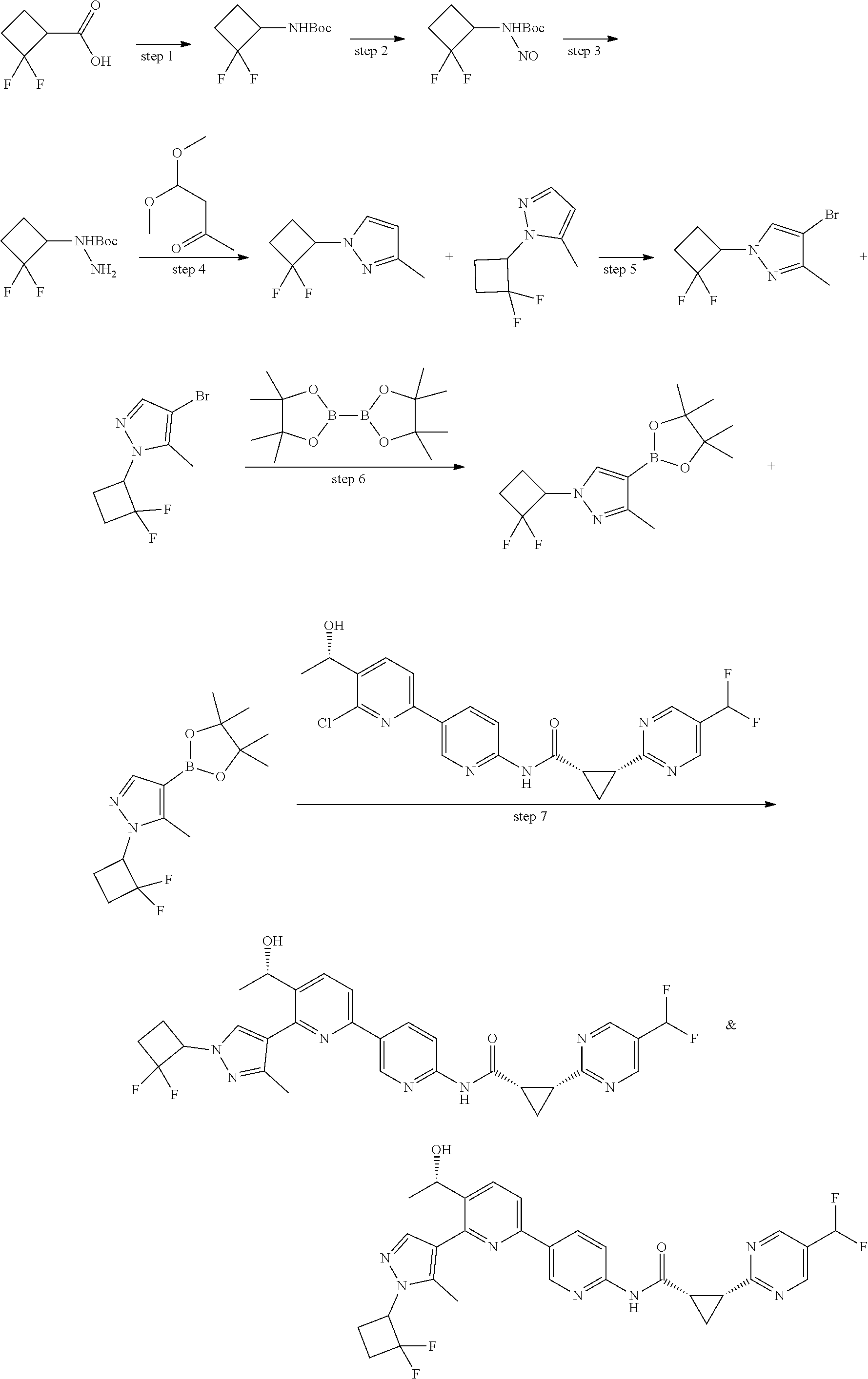

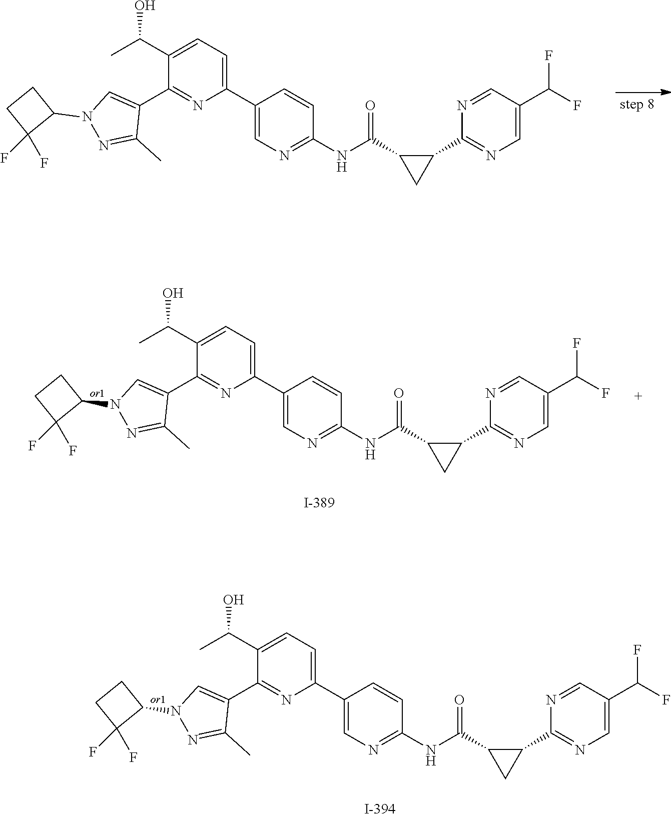 [SMILES: Cc1nn(C2CCC2(F)F)cc1-c1nc(-c2ccc(NC(=O)[C@H]3C[C@H]3c3ncc(C(F)F)cn3)nc2)ccc1[C@H](C)O ; Cc1nn([C@@H]2CCC2(F)F)cc1-c1nc(-c2ccc(NC(=O)[C@H]3C[C@H]3c3ncc(C(F)F)cn3)nc2)ccc1[C@H](C)O ; Cc1nn([C@H]2CCC2(F)F)cc1-c1nc(-c2ccc(NC(=O)[C@H]3C[C@H]3c3ncc(C(F)F)cn3)nc2)ccc1[C@H](C)O]

### Step 1. Synthesis of tert-butyl (2,2-difluorocyclobutyl) carbamate

A solution of 2,2-difluorocyclobutane-1-carboxylic acid (3.20 g, 23.51 mmol, 1 eq) and Et₃N (2.62 g, 25.86 mmol, 1.1 eq) in t-BuOH (50 mL) was stirred at 25° C. Then DPPA (7.12 g, 25.86 mmol, 1.1 eq) was added to the mixture. The resulting mixture was stirred at 80° C. for 16 h under N₂ atmosphere. The reaction mixture was concentrated under reduced pressure, diluted with water (200 mL) and extracted with EtOAc (150 mL*3). The combined organic layers were washed with brine (100 mL*2), dried over anhydrous Na₂SO₄, filtered and the filtrate was concentrated under reduced pressure. The residue was purified by flash silica gel chromatography (Eluent of PE/EtOAc) to afford the title compound. ¹H NMR (400 MHz, CDCl₃) δ=4.96 (d, 1H), 4.56 (d, 1H), 2.38-2.21 (m, 3H), 1.56 (t, 1H), 1.46 (s, 9H).

### Step 2. Synthesis of tert-butyl (2,2-difluorocyclobutyl)(nitroso)carbamate

To a solution of tert-butyl (2,2-difluorocyclobutyl) carbamate (3.20 g, 15.44 mmol, 1 eq) in pyridine (4 mL) and ACN (40 mL) was added nitrosonium tetrafluoroborate (2.71 g, 23.16 mmol, 1.5 eq) at −30° C. under N₂. The resulting mixture was stirred at −30° C. for 0.5 h, then warmed up to 0° C. and stirred at 0° C. for 10 minutes. The reaction mixture was diluted with water (100 mL) and then extracted with EtOAc (50 mL*3). The combined organic layers were washed with water (50 mL), dried over anhydrous Na₂SO₄, filtered and the filtrate was concentrated under reduced pressure. The residue was purified by flash silica gel chromatography (Eluent of PE/EtOAc) to afford the title compound. ¹H NMR (400 MHz, CDCl₃) δ=5.21-5.02 (m, 1H), 2.79-2.60 (m, 1H), 2.55-2.34 (m, 2H), 2.33-2.17 (m, 1H), 1.66 (s, 9H).

### Step 3. Synthesis of tert-butyl 1-(2,2-difluorocyclobutyl)hydrazine-1-carboxylate

To a solution of tert-butyl (2,2-difluorocyclobutyl)(nitroso)carbamate (2.80 g, 11.85 mmol, 1 eq) in DCM (30 mL) was added AcOH (14.24 g, 237.07 mmol, 20 eq). The resulting mixture was cooled to 0° C. Then zinc powder (7.75 g, 118.54 mmol, 10 eq) was added to the mixture in 5 batches at 0° C. under N₂. The reaction mixture was stirred at 0° C. for 0.5 h and then filtered through a celite pad. The filter cake was washed with DCM (30 mL*2). The combined filtrate was diluted with H₂O (100 mL) and then extracted with DCM (50 mL*3). The combined organic phase was washed with brine (50 mL), dried over anhydrous Na₂SO₄, filtered and the filtrate was concentrated under reduced pressure. The residue was purified by flash silica gel chromatography (Eluent of PE/EtOAc) to afford the title compound.

### Step 4. Synthesis of a mixture of 1-(2,2-difluorocyclobutyl)-3-methyl-1H-pyrazole and 1-(2,2-difluorocyclobutyl)-5-methyl-1H-pyrazole

To a solution of tert-butyl 1-(2,2-difluorocyclobutyl)hydrazine-1-carboxylate (2.12 g, 8.20 mmol, 1 eq) and 4,4-dimethoxybutan-2-one (1.08 g, 8.20 mmol, 1.09 mL, 1 eq) in EtOH (25 mL) was added aq. HCl (5 M, 1.97 mL, 1.2 eq). The resulting mixture was stirred at 90° C. for 1 hour. The reaction mixture was basified by sodium bicarbonate aqueous solution (50 mL) to pH-8 and extracted with EtOAc (50 mL*3). The combined organic layers were washed with brine (100 mL*2), dried over anhydrous Na₂SO₄, filtered and the filtrate was concentrated under reduced pressure to afford the title compounds. LCMS: 173.3 [M+H]⁺

### Step 5. Synthesis of a mixture of 4-bromo-1-(2,2-difluorocyclobutyl)-3-methyl-1H-pyrazole and 4-bromo-1-(2,2-difluorocyclobutyl)-5-methyl-1H-pyrazole

To a solution of a mixture of 1-(2,2-difluorocyclobutyl)-3-methyl-1H-pyrazole and 1-(2,2-difluorocyclobutyl)-5-methyl-1H-pyrazole from previous step (1.8 g, 10.45 mmol, 1 eq) in DCM (30 mL) was added NBS (2.05 g, 11.50 mmol, 1.1 eq). The resulting mixture was stirred at 40° C. for 1 hour. The reaction mixture was diluted with water (50 mL) and extracted with DCM (50 mL*3). The combined organic layers were washed with brine (10 mL*2), dried over anhydrous Na₂SO₄, filtered and the filtrate was concentrated under reduced pressure. The residue was purified by flash silica gel chromatography (Eluent of PE/EtOAc) to afford the title compounds. LCMS: 250.9 [M+H]⁺

### Step 6. Synthesis of a mixture of 1-(2,2-difluorocyclobutyl)-3-methyl-4-(4,4,5,5-tetramethyl-1,3,2-dioxaborolan-2-yl)-1H-pyrazole and 1-(2,2-difluorocyclobutyl)-5-methyl-4-(4,4,5,5-tetramethyl-1,3,2-dioxaborolan-2-yl)-1H-pyrazole

A mixture of 4-bromo-1-(2,2-difluorocyclobutyl)-3-methyl-1H-pyrazole and 4-bromo-1-(2,2-difluorocyclobutyl)-5-methyl-1H-pyrazole from previous step (630 mg, 2.51 mmol, 1 eq), 4,4,4′,4′,5,5,5′,5′-octamethyl-2,2′-bi(1,3,2-dioxaborolane) (765 mg, 3.01 mmol, 1.2 eq), PCy3-Pd-G3 (184 mg, 0.25 mmol, 0.1 eq) and KOAc (493 mg, 5.02 mmol, 2 eq) in toluene (10 mL) was degassed and purged with N₂ 3 times, and then the resulting mixture was stirred at 90° C. for 1 h under N₂ atmosphere. The reaction mixture was cooled to room temperature, diluted with water (20 mL) and extracted with EtOAc (10 mL*3). The combined organic layers were washed with brine (10 mL*2), dried over anhydrous Na₂SO₄, filtered and the filtrate was concentrated under reduced pressure to give a residue. The residue was purified by flash silica gel chromatography (Eluent of PE/EtOAc) and reverse phase preparative HPLC (ACN/H₂O, 0.05% NH₃·H₂O) to afford the title compounds. LCMS: 299.2 [M+H]⁺

### Step 7. Synthesis of (1S,2R)—N-(6-(1-(2,2-difluorocyclobutyl)-3-methyl-1H-pyrazol-4-yl)-5-((S)-1-hydroxyethyl)-[2,3′-bipyridin]-6′-yl)-2-(5-(difluoromethyl) pyrimidin-2-yl) cyclopropane-1-carboxamide & (1S,2R)—N-(6-(1-(2,2-difluorocyclobutyl)-5-methyl-1H-pyrazol-4-yl)-5-((S)-1-hydroxyethyl)-[2,3′-bipyridin]-6′-yl)-2-(5-(difluoromethyl) pyrimidin-2-yl) cyclopropane-1-carboxamide

To a solution of (1S,2R)—N-(6-chloro-5-((S)-1-hydroxyethyl)-[2,3′-bipyridin]-6′-yl)-2-(5-(difluoromethyl)pyrimidin-2-yl)cyclopropane-1-carboxamide (obtained as described in step 4 of I-150 synthesis) (100 mg, 0.22 mmol, 1 eq) in dioxane (2 mL) and H₂O (0.2 mL) was added a mixture of 1-(2,2-difluorocyclobutyl)-5-methyl-4-(4,4,5,5-tetramethyl-1,3,2-dioxaborolan-2-yl)-1H-pyrazole) and 1-(2,2-difluorocyclobutyl)-3-methyl-4-(4,4,5,5-tetramethyl-1,3,2-dioxaborolan-2-yl)-1H-pyrazole from previous step (160 mg, 0.27 mmol, 1.2 eq), Pd(dtbpf)Cl₂ (15 mg, 22 mol, 0.1 eq) and Et₃N (68 mg, 0.67 mmol, 3 eq). The resulting mixture was stirred at 100° C. for 0.5 hour. The reaction mixture was filtered through celite and the filtrate was concentrated under reduced pressure. The residue was purified by reverse phase preparative HPLC (ACN/H₂O, 0.05% NH₃·H₂O) to afford the title compound. LCMS: 582.2 [M+H]⁺

### Step 8. Separation of (1S,2R)—N-(6-(1-(2,2-difluorocyclobutyl)-3-methyl-1H-pyrazol-4-yl)-5-((S)-1-hydroxyethyl)-[2,3′-bipyridin]-6′-yl)-2-(5-(difluoromethyl) pyrimidin-2-yl) cyclopropane-1-carboxamide

The steroisomers of (1S,2R)—N-(6-(1-(2,2-difluorocyclobutyl)-3-methyl-1H-pyrazol-4-yl)-5-((S)-1-hydroxyethyl)-[2,3′-bipyridin]-6′-yl)-2-(5-(difluoromethyl) pyrimidin-2-yl) cyclopropane-1-carboxamide were separated by chiral SFC (SFC Preparative Method: Instrument: Waters 150 Preparative SFC system; column: DAICEL CHIRALPAK® AD (250 mm*30 mm, 10 m); Mobile phase A: supercritical CO₂, Mobile Phase B: EtOH:CAN=4:1 (0.1% NH₃—H₂O); Isocratic elution: 40% Phase B in Supercritical CO₂; Flow rate: 120 g/min; Retention time: 2.87 min, 3.98 min. Back Pressure: 80 bar to keep the CO₂ in Supercritical flow; Wave Length: 220 nm) to give two peaks. Peak 1 (Rt=2.87 min) is (1S,2R)—N-(6-{1-[(1R*)-2,2-difluorocyclobutyl]-3-methyl-1H-pyrazol-4-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 1) (I-389); Peak 2 (Rt=3.98 min) was further purified by reverse phase preparative HPLC (ACN/H₂O, 0.05% NH₄HCO₃) to afford (1S,2R)—N-(6-{1-[(1R*)-2,2-difluorocyclobutyl]-3-methyl-1H-pyrazol-4-yl}-5-[(1S)-1-hydroxyethyl]-[2,3′-bipyridin]-6′-yl)-2-[5-(difluoromethyl)pyrimidin-2-yl]cyclopropane-1-carboxamide (stereoisomer 2) (I-394).

Analytical data for stereoisomer 1 (I-389): ¹H NMR (400 MHz, CD₃OD) δ=8.91 (d, 1H), 8.82 (s, 2H), 8.33-8.24 (m, 1H), 8.11 (d, 1H), 7.92 (t, 2H), 7.83 (d, 1H), 6.92 (t, 1H), 5.32-5.18 (m, 1H), 4.98-4.93 (m, 1H), 2.99-2.88 (m, 1H), 2.65-2.43 (m, 5H), 2.32 (s, 3H), 2.13-2.02 (m, 1H), 1.64-1.55 (m, 1H), 1.42 (d, 3H). LCMS: 582.3 [M+H]⁺. Analytical SFC: R^{†}=1.570 min

Analytical data for stereoisomer 2 (I-394): ¹H NMR (400 MHz, CD₃OD) δ=8.91 (s, 1H), 8.82 (s, 2H), 8.35-8.25 (m, 1H), 8.11 (d, 1H), 7.97-7.89 (m, 2H), 7.83 (d, 1H), 6.92 (t, 1H), 5.34-5.17 (m, 1H), 4.98 (d, 1H), 2.99-2.88 (m, 1H), 2.61-2.43 (m, 5H), 2.32 (s, 3H), 2.13-2.04 (m, 1H), 1.64-1.54 (m, 1H), 1.41 (d, 3H). LCMS: 582.2 [M+H]⁺. Analytical SFC: Rt=1.641 min

Analytical SFC method:

| Instrument | SHIMADZU LC-30ADsf | |
|---|---|---|
| Column | CHIRALPAK ® IG-3 50 × 4.6 mm I.D., particle | |
| | size 3 μm | |
| Mobile Phase | Phase A for CO₂ | Phase B for EtOH:ACN 2:1 |
| | | (+0.05% diethylamine) |

| Gradient | Time(min) | A(%) | B(%) |
|---|---|---|---|
| | 0.00 | 80 | 20 |
| | 1.80 | 40 | 60 |
| | 2.70 | 40 | 60 |
| | 2.71 | 80 | 20 |
| | 3.00 | 80 | 20 |

| | |
|---|---|
| Flow rate | 3.0 mL/min |
| Column Temperature | 35° C. |
| Back pressure | 10 MPa |
| UV wave length | 220 nm |

### Example 53. Synthesis of (1S,2R)-2-(5-(difluoromethyl)pyridin-2-yl)-N-(5-((S)-1-hydroxyethyl)-6-methoxy-4′-(methylamino)-[2,3′-bipyridin]-6′-yl)cyclopropane-1-carboxamide (I-352)

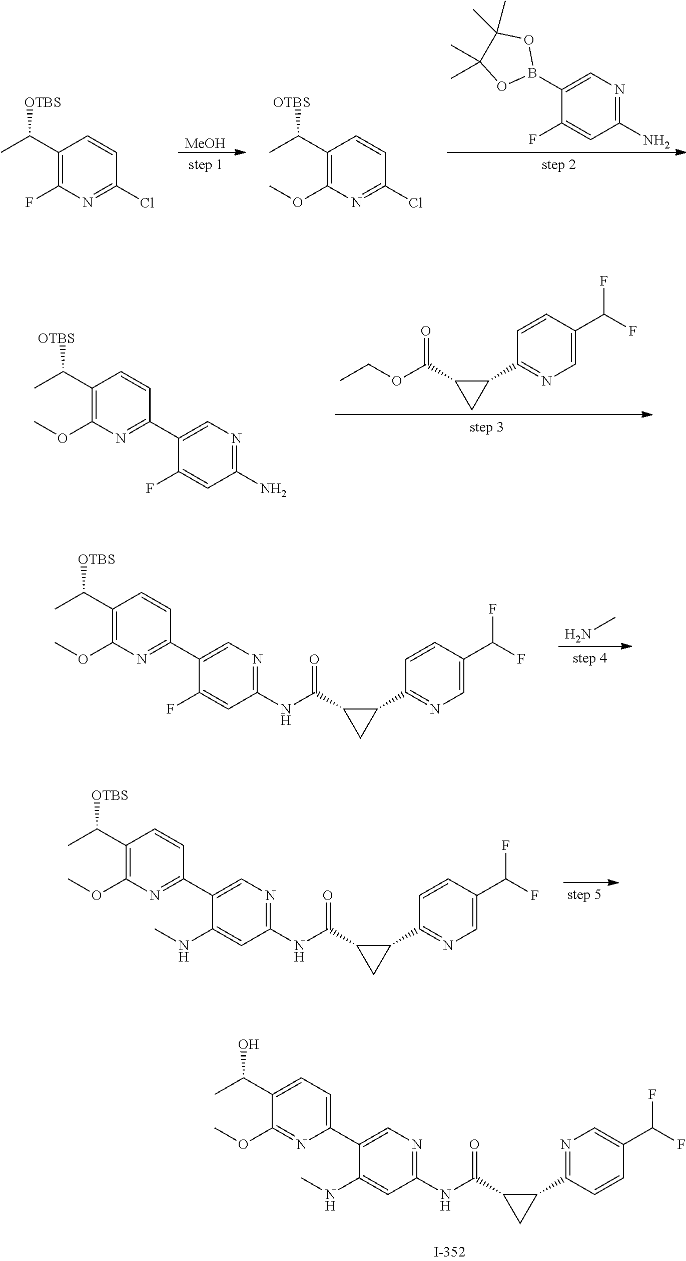

### Step 1. Synthesis of (S)-3-(1-((tert-butyldimethylsilyl)oxy)ethyl)-6-chloro-2-methoxypyridine

To a solution of MeOH (22 mg, 690 mol, 2.0 eq) in THF (2 mL) was added NaH (28 mg, 690 mol, 60% in mineral oil, 2.0 eq) at 0° C., then (S)-3-(1-((tert-butyldimethylsilyl)oxy)ethyl)-6-chloro-2-fluoropyridine (prepared from (S)-1-(6-chloro-2-fluoropyridin-3-yl)ethan-1-ol (step 1 of I-11) by analogy to step 2 of I-150 synthesis) (100 mg, 345 mol, 1.0 eq) was added, and the resulting mixture was stirred at 40° C. for 0.5 h under N₂ atmosphere. The reaction mixture was quenched with H₂O (10 mL) and extracted with EtOAc (10 mL*2). The combined organic phase was washed with brine (10 mL), dried over anhydrous Na₂SO₄, filtered and the filtrate was concentrated under reduced pressure to afford the tittle compound, which was used into the next step without further purification. LCMS: 302.2 [M+H]⁺

### Step 2. Synthesis of (S)-5-(1-((tert-butyldimethylsilyl)oxy)ethyl)-4′-fluoro-6-methoxy-[2,3′-bipyridin]-6′-amine

To a solution of (S)-3-(1-((tert-butyldimethylsilyl)oxy)ethyl)-6-chloro-2-methoxypyridine (104 mg, 344 mol, 1.0 eq) and 4-fluoro-5-(4,4,5,5-tetramethyl-1,3,2-dioxaborolan-2-yl)pyridin-2-amine (180 mg, 756 mol, 2.2 eq) in 1,4-dioxane (3 mL) and H₂O (0.6 mL) was added Pd(dppf)C12 (25 mg, 34 mol, 0.1 eq) and K₃PO₄ (146 mg, 689 mol, 2.0 eq), and the resulting mixture was stirred at 60° C. overnight under N₂ atmosphere. The reaction mixture was diluted H₂O (10 mL), extracted with EtOAc (10 mL*2), the combined organic phase was washed with brine (10 mL), dried over anhydrous Na₂SO₄, filtered and the filtrate was concentrated under reduced pressure. The residue was purified by flash silica gel chromatography (Eluent of EtOAc/PE) to afford the title compound. LCMS: 378.1 [M+H]⁺

### Step 3. Synthesis of (1S,2R)—N-(5-((S)-1-((tert-butyldimethylsilyl)oxy)ethyl)-4′-fluoro-6-methoxy-[2,3′-bipyridin]-6′-yl)-2-(5-(difluoromethyl)pyridin-2-yl)cyclopropane-1-carboxamide

To a solution of (S)-5-(1-((tert-butyldimethylsilyl)oxy)ethyl)-4′-fluoro-6-methoxy-[2,3′-bipyridin]-6′-amine (150 mg, 397 mol, 1.0 eq) and ethyl (1S,2R)-2-(5-(difluoromethyl)pyridin-2-yl)cyclopropane-1-carboxylate (192 mg, 795 mol, 2.0 eq) in THF (3 mL) was added AlMe₃ (2 M in THF, 993 μL, 5.0 eq), and the reaction mixture was stirred at 60° C. overnight under N₂ atmosphere. The reaction mixture was quenched with sat. aq. NH₄Cl (10 mL) and H₂O (10 mL), the resulting mixture was extracted with EtOAc (10 mL*2), the combined organic phase was washed with brine (10 mL), dried over anhydrous Na₂SO₄, filtered and the filtrate was concentrated under reduced pressure. The residue was purified by flash silica gel chromatography (Eluent of EtOAc/PE) to afford the title compound. LCMS: 573.1 [M+H]⁺

### Step 4. Synthesis of (1S,2R)—N-(5-((S)-1-((tert-butyldimethylsilyl)oxy)ethyl)-6-methoxy-4′-(methylamino)-[2,3′-bipyridin]-6′-yl)-2-(5-(difluoromethyl)pyridin-2-yl)cyclopropane-1-carboxamide

To a solution of (1S,2R)—N-(5-((S)-1-((tert-butyldimethylsilyl)oxy)ethyl)-4′-fluoro-6-methoxy-[2,3′-bipyridin]-6′-yl)-2-(5-(difluoromethyl)pyridin-2-yl)cyclopropane-1-carboxamide (70 mg, 122 mol, 1.0 eq) and methylamine hydrochloride (41 mg, 611 mol, 5.0 eq) in DMA (2 mL) was added DIEA (158 mg, 1.22 mmol, 10.0 eq), and the reaction mixture was stirred at 100° C. for 4 hours. The reaction mixture was used into the next step without work-up and purification. LCMS: 584.3 [M+H]⁺

### Step 5. Synthesis of (1S,2R)-2-(5-(difluoromethyl)pyridin-2-yl)-N-(5-((S)-1-hydroxyethyl)-6-methoxy-4′-(methylamino)-[2,3′-bipyridin]-6′-yl)cyclopropane-1-carboxamide

To the reaction mixture of step 4 was added CsF (370 mg, 2.43 mmol, 20.0 eq), and the resulting mixture was stirred at 100° C. for 0.5 hour. The reaction mixture was concentrated under reduced pressure. The residue was purified by reverse phase HPLC (C18 column, water (0.1% FA)-ACN) to afford the title compound (as formic acid salt). LCMS: 470.1 [M+H]⁺. ¹H NMR (400 MHz, DMSO-d6) δ 10.49 (s, 1H), 8.61 (s, 1H), 8.41-8.33 (m, 1H), 8.28 (s, 1H), 8.18 (s, 1H), 7.88 (br d, 1H), 7.83 (d, 1H), 7.46 (d, 1H), 7.42 (d, 1H), 7.24-6.93 (m, 2H), 5.26 (s, 1H), 4.92-4.81 (m, 1H), 3.90 (s, 3H), 2.72 (d, 3H), 2.59-2.52 (m, 1H), 1.84-1.72 (m, 1H), 1.47-1.38 (m, 1H), 1.29 (d, 3H)

### Example 54. Synthesis of (1R*)-1-[5-(difluoromethyl)pyrimidin-2-yl]ethyl N-{5-[(1S)-1-hydroxyethyl]-6-[3-methyl-1-(2,2,2-trifluoroethyl)-1H-pyrazol-4-yl]-[2,3′-bipyridin]-6′-yl}carbamate (stereoisomer 1) (I-360) and (1R*)-1-[5-(difluoromethyl)pyrimidin-2-yl]ethyl N-{5-[(1S)-1-hydroxyethyl]-6-[3-methyl-1-(2,2,2-trifluoroethyl)-1H-pyrazol-4-yl]-[2,3′-bipyridin]-6′-yl}carbamate (stereoisomer 2) (I-361)

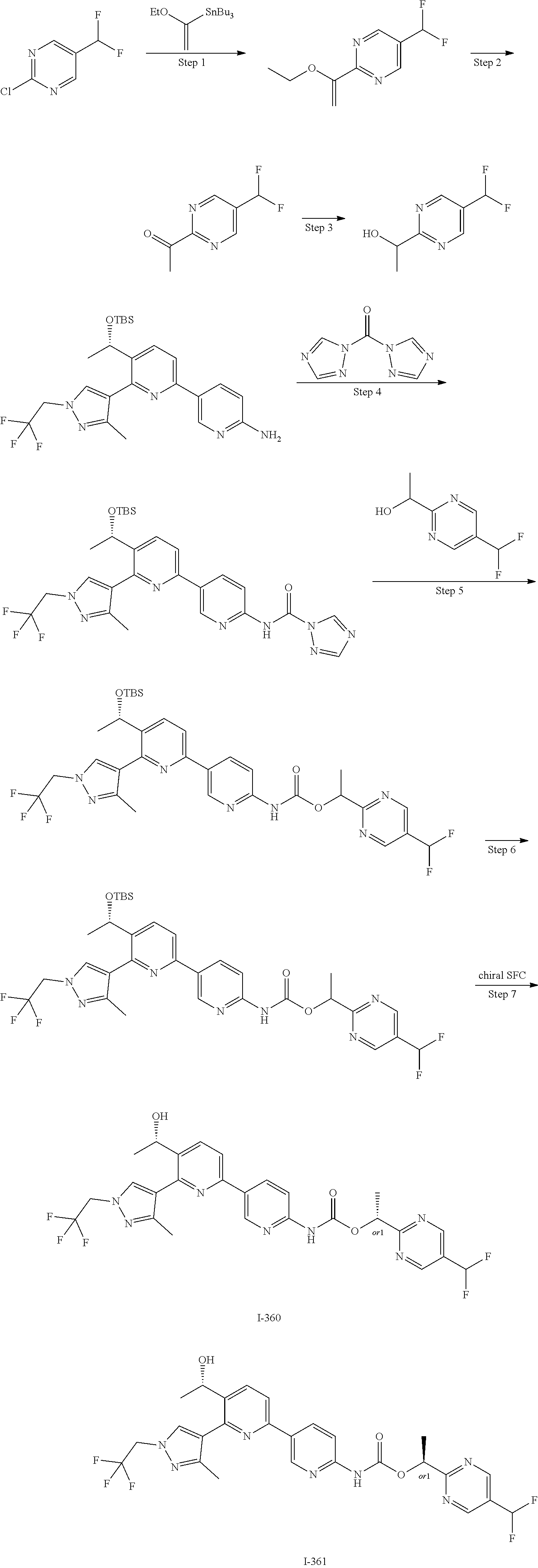

### Step 1. Synthesis of 5-(difluoromethyl)-2-(1-ethoxyvinyl)pyrimidine

To a solution of 2-chloro-5-(difluoromethyl) pyrimidine (1.70 g, 10.33 mmol, 1 eq) in DMF (20 mL) was added Pd(dppf)C12 (725 mg, 1.03 mmol, 0.1 eq) and tributyl(1-ethoxyvinyl)stannane (4.88 g, 13.51 mmol, 1.31 eq). The resulting mixture was stirred at 100° C. for 3 h under N₂ atmosphere. The reaction mixture was cooled to 25° C., poured into water (80 mL) and extracted with EtOAc (80 mL×3). The combined organic layers were washed with brine (100 mL×3), dried over anhydrous Na₂SO₄, filtered and the filtrate was concentrated under reduced pressure to give a residue. The residue was purified by flash silica gel chromatography (Eluent of EtOAc/PE) to afford the title compound. LCMS: 201.0 [M+H]⁺

### Step 2. Synthesis of 1-(5-(difluoromethyl)pyrimidin-2-yl)ethan-1-one

To a solution of 5-(difluoromethyl)-2-(1-ethoxyvinyl)pyrimidine (1.81 g, 9.04 mmol, 1 eq) in THF (20 mL) was added aq. HCl (2 M, 9.04 mL, 2 eq) at 20° C. The resulting mixture was stirred at 20° C. for 2 hours. The pH of the reaction mixture was then adjusted to pH=8 by addition of sat. aq. NaHCO₃ solution. The organic layer was separated and the aqueous layer was extracted with EtOAc (30 mL×3). The combined organic layers were dried over anhydrous Na₂SO₄, filtered and the filtrate was concentrated under reduced pressure to afford the title compound which was used in the next step without additional purification. LCMS: 173.0 [M+H]⁺

### Step 3. Synthesis of 1-(5-(difluoromethyl)pyrimidin-2-yl)ethan-1-ol

To a solution of 1-(5-(difluoromethyl)pyrimidin-2-yl)ethan-1-one (1.90 g, 11.04 mmol, 1 eq) in THF (20 mL) was added NaBH₄ (146 mg, 3.86 mmol, 0.35 eq) at 0° C. under N₂ atmosphere. The reaction mixture was stirred at 0° C. for 5 min, then poured into sat. aq. NH₄Cl (30 mL) and extracted with EtOAc (30 mL×3). The combined organic layers were washed with brine (100 mL), dried over anhydrous Na₂SO₄, filtered and the filtrate was concentrated under reduced pressure. The residue was purified by flash silica gel chromatography (Eluent of EtOAc/PE) to afford the title compound. LCMS: 157.2 [M−H₂O+H]⁺

### Step 4. Synthesis of N-[5-[5-[(1S)-1-[tert-butyl(dimethyl)silyl]oxyethyl]-6-[3-methyl-1-(2,2,2-trifluoroethyl)pyrazol-4-yl]-2-pyridyl]-2-pyridyl]-1,2,4-triazole-1-carboxamide

To a solution of 5-[5-[(1S)-1-[tert-butyl(dimethyl)silyl]oxyethyl]-6-[3-methyl-1-(2,2,2-trifluoroethyl)pyrazol-4-yl]-2-pyridyl]pyridin-2-amine (obtained as described in step 7 of I-152 synthesis) (90 mg, 183 mol, 1.0 eq) and TEA (56 mg, 549 mol, 3.0 eq) in DMA (3 mL) was added bis(1,2,4-triazol-1-yl)methanone (90 mg, 549 mol, 3.0 eq) at 0° C. The resulting mixture was stirred at 25° C. for 2 h. The reaction solution was used into the next step without further purification.

### Step 5. Synthesis of [1-[5-(difluoromethyl)pyrimidin-2-yl]ethyl]N-[5-[5-[(1S)-1-[tertbutyl(dimethyl)silyl]oxyethyl]-6-[3-methyl-1-(2,2,2-trifluoroethyl)pyrazol-4-yl]-2-pyridyl]-2-pyridyl]carbamate

To the solution obtained in step 4 was added 1-[5-(difluoromethyl)pyrimidin-2-yl]ethanol (obtained in step 3) (30 mg, 170 mol, 0.93 eq) in DMA (3 mL) and TEA (52 mg, 511 μmol, 2.8 eq). The resulting mixture was stirred at 0° C. for 3 hours. The reaction mixture was diluted with H₂O (8 mL) and extracted with EtOAc (4 mL*3). The combined organic layers were washed with brine (5 mL*2), dried over anhydrous Na₂SO₄, filtered and the filtrate was concentrated under reduced pressure. The residue was purified by flash silica gel chromatography (Eluent of EtOAc/PE) to afford the title compound. LCMS: 691.7 [M+H]⁺

### Step 6. Synthesis of 1-(5-(difluoromethyl)pyrimidin-2-yl)ethyl (5-((S)-1-hydroxyethyl)-6-(3-methyl-1-(2,2,2-trifluoroethyl)-1H-pyrazol-4-yl)-[2,3′-bipyridin]-6′-yl)carbamate

To a solution of [1-[5-(difluoromethyl)pyrimidin-2-yl]ethyl]N-[5-[5-[(1S)-1-[tert-butyl(dimethyl)silyl]oxyethyl]-6-[3-methyl-1-(2,2,2-trifluoroethyl)pyrazol-4-yl]-2-pyridyl]-2-pyridyl]carbamate (40 mg, 58 mol, 1 eq) in THF (1 mL) was added TBAF (1 M in THF, 116 μL, 2.0 eq) at 0° C. The resulting mixture was stirred at 0° C. for 2 h. The reaction mixture was concentrated under reduced pressure. The residue was purified by reverse phase preparative HPLC (H₂O (0.225% FA)-ACN) to afford the title compound. ¹H NMR (400 MHz, CD₃OD) δ=8.97 (s, 2H), 8.91 (d, 1H), 8.38-8.31 (m, 1H), 8.12 (d, 1H), 7.94-7.90 (m, 2H), 7.85 (d, 1H), 7.19-6.81 (m, 1H), 5.89 (d, 1H), 4.97-4.93 (m, 3H), 2.33 (s, 3H), 1.70 (d, 3H), 1.42 (d, 3H). LCMS: 578.2 [M+H]⁺

### Step 7. Chiral separation of [1-[5-(difluoromethyl)pyrimidin-2-yl]ethyl]N-[5-[5-[(1S)-1-hydroxyethyl]-6-[3-methyl-1-(2,2,2-trifluoroethyl)pyrazol-4-yl]-2-pyridyl]-2-pyridyl]carbamate

The mixture of the two stereoisomers was separated by chiral SFC (SFC Preparative Method: Instrument: Waters 80Q Preparative SFC system; Column: DAICEL CHIRALPAK® AD 250×25 mm I.D., 10 m; Mobile phase A: supercritical CO₂, Mobile Phase B: IPA:ACN 4:1 (0.1% NH₃*H₂O); Isocratic elution: 60% Phase B in Supercritical CO₂; Flow rate: 80 g/min; Retention time: Peak1: 2.03 min, Peak2: 2.58 min; Back Pressure: 100 bar to keep the CO₂ in Supercritical flow; Wave Length: 220 nm).

Peak 1 (first eluting): (1R*)-1-[5-(difluoromethyl)pyrimidin-2-yl]ethyl N-{5-[(1S)-1-hydroxyethyl]-6-[3-methyl-1-(2,2,2-trifluoroethyl)-1H-pyrazol-4-yl]-[2,3′-bipyridin]-6′-yl}carbamate (stereoisomer 1) (I-360); ¹H NMR (400 MHz, CD₃OD) δ=9.00-8.84 (m, 3H), 8.40-8.29 (m, 1H), 8.13 (d, 1H), 7.95-7.89 (m, 2H), 7.86 (d, 1H), 7.34-6.75 (m, 1H), 5.97-5.84 (m, 1H), 5.02-4.96 (m, 3H), 2.34 (s, 3H), 1.70 (d, 3H), 1.42 (d, 3H). LCMS: 578.3 [M+H]⁺. Analytical chiral SFC: Rt=1.042 min.

Peak 2 (second eluting): (1R*)-1-[5-(difluoromethyl)pyrimidin-2-yl]ethyl N-{5-[(1S)-1-hydroxyethyl]-6-[3-methyl-1-(2,2,2-trifluoroethyl)-1H-pyrazol-4-yl]-[2,3′-bipyridin]-6′-yl}carbamate (stereoisomer 2) (I-361); ¹H NMR (400 MHz, CD₃OD) δ=8.99-8.84 (m, 3H), 8.45-8.33 (m, 1H), 8.14 (d, 1H), 7.97-7.91 (m, 2H), 7.88 (d, 1H), 7.20-6.81 (m, 1H), 5.98-5.85 (m, 1H), 5.02-4.96 (m, 3H), 2.36 (s, 3H), 1.70 (d, 3H), 1.44 (d, 3H). LCMS: 578.3 [M+H]⁺. Analytical chiral SFC: Rt=1.223 min.

### Analytical Chiral SFC Method:

| Instrument | SHIMADZU LC-30ADsf | |
|---|---|---|
| column | CHIRALPAK ® AD-3 50 × 4.6 mm I.D., particle | |
| | size 3 μm | |
| Mobile Phase | Phase A for CO₂ | Phase B for 2:1 IPA + ACN |
| | | (0.05% DEA) |

| Gradient | Time(min) | A(%) | B(%) |
|---|---|---|---|
| | 0.00 | 70 | 30 |
| | 1.20 | 40 | 60 |
| | 1.70 | 40 | 60 |
| | 1.71 | 70 | 30 |
| | 2.00 | 70 | 30 |

| | |
|---|---|
| Flow rate | 4.0 mL/min |
| Column Temp | 35° C. |
| Back pressure | 100 Bar |
| UV | 220 nm |

### Example 55. Synthesis of 1-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-3-[(1R)-1-[5-(difluoromethyl)pyrazin-2-yl]ethyl]urea (stereoisomer 1) (I-318) and 1-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-3-[(1R)-1-[5-(difluoromethyl)pyrazin-2-yl]ethyl]urea (stereoisomer 2) (I-319)

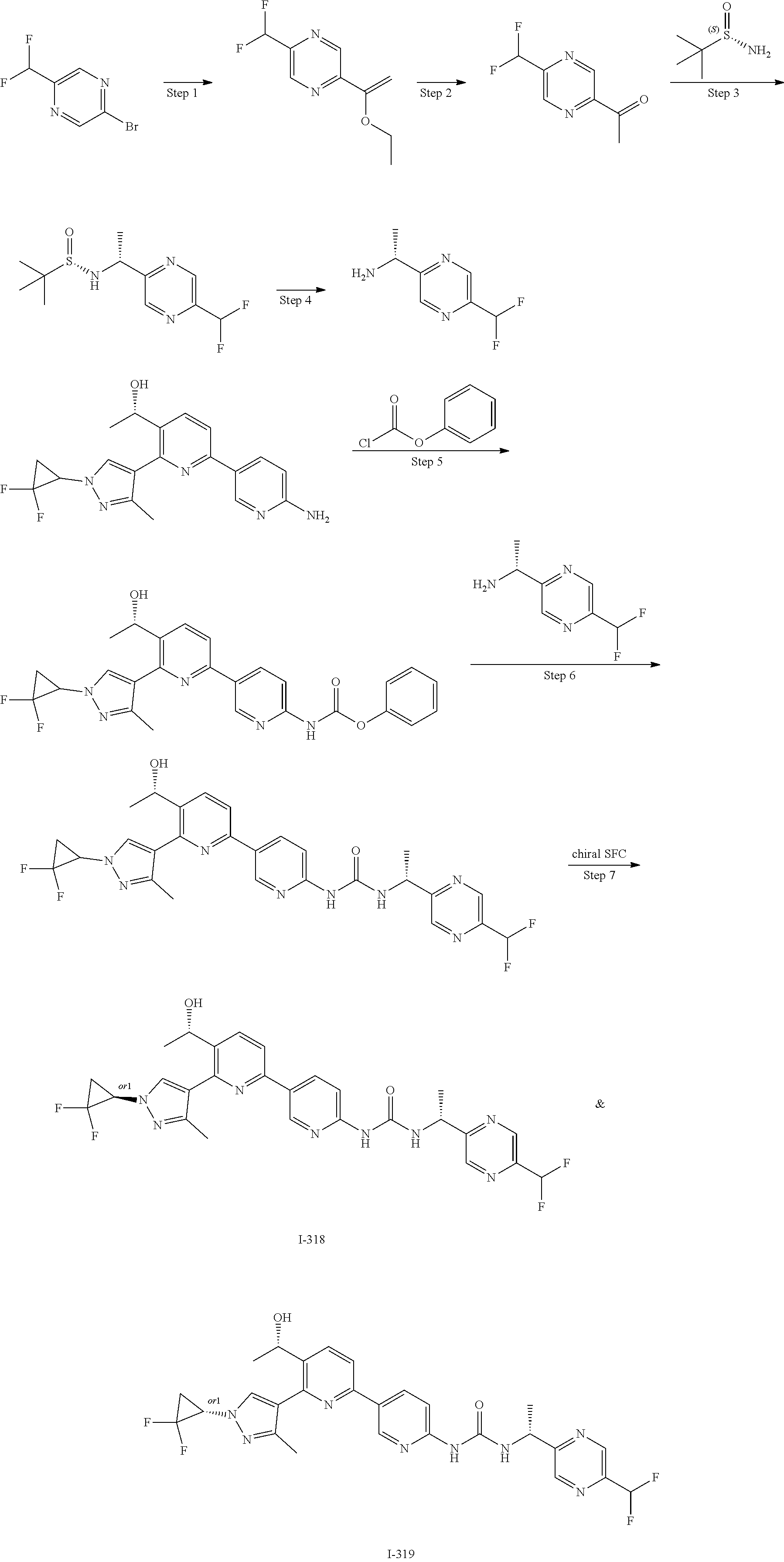

### Step 1. Synthesis of 2-(difluoromethyl)-5-(1-ethoxyvinyl)pyrazine

To a stirred solution of 2-bromo-5-(difluoromethyl) pyrazine (400 mg, 1.91 mmol) and tributyl(1-ethoxyvinyl) stannane (1.38 g, 3.83 mmol) in toluene (10 mL) was added Pd(PPh₃)₂Cl₂ (0.13 g, 0.191 mmol) at room temperature under N₂ atmosphere. The resulting mixture was stirred at 100° C. for 2 h under N₂ atmosphere. The reaction mixture was allowed to cool down to room temperature and then concentrated under reduced pressure. The crude product was used in the next step directly without further purification. LCMS: 201 [M+H]⁺

### Step 2. Synthesis of 1-(5-(difluoromethyl)pyrazin-2-yl) ethan-1-one

To a stirred solution of 2-(difluoromethyl)-5-(1-ethoxyvinyl) pyrazine (400 mg, 2.00 mmol) in toluene (10 mL) was added aq. HCl (37 wt %, 0.32 mL, 37% Wt) at 0° C. under N₂ atmosphere. The resulting mixture was stirred at room temperature overnight under N₂ atmosphere. The reaction mixture was diluted with water (30 mL) and extracted with CH₂Cl₂ (3×30 mL). The combined organic phase was dried over Na₂SO₄, filtered and the filtrate was concentrated under reduced pressure. The residue was purified by Prep-TLC (petroleum ether/EtOAc) to afford the title compound. LCMS: 173 [M+H]⁺

### Step 3. Synthesis of (S)—N—((R)-1-(5-(difluoromethyl)pyrazin-2-yl)ethyl)-2-methylpropane-2-sulfinamide

To a stirred solution of 1-(5-(difluoromethyl)pyrazin-2-yl)ethan-1-one (320 mg, 1.86 mmol) and (S)-2-methylpropane-2-sulfinamide (455 mg, 3.76 mmol) in diethyl ether (15 mL) was added Ti(i-PrO)₄ (1.07 g, 3.76 mmol) at room temperature. The reaction mixture was stirred at 40° C. overnight, then lithium tri-sec-butylhydroborate (1M in THF, 0.28 mL, 0.268 mmol) was added at −78° C. under N₂ atmosphere and the reaction mixture was stirred at −78° C. for 2 h. The reaction mixture was warmed up to room temperature and stirred for 1 h, and then concentrated under reduced pressure. The residue was purified by Prep-TLC (EtOAc) to afford the title compound. LCMS: 278 [M+H]⁺

### Step 4. Synthesis of (R)-1-(5-(difluoromethyl)pyrazin-2-yl)ethan-1-amine

To a stirred solution of (S)—N—((R)-1-(5-(difluoromethyl)pyrazin-2-yl)ethyl)-2-methylpropane-2-sulfinamide (200 mg, 0.721 mmol) in dioxane (5 mL) was added HCl solution in 1,4-dioxane (4 M, 0.5 mL, 0.023 mmol) dropwise at 0° C., and the resulting mixture was stirred at room temperature for 1 h under N₂ atmosphere. The reaction mixture was concentrated under reduce pressure to provide the title compound which was used in the next step without further purification. LCMS: 174 [M+H]⁺

### Step 5. Synthesis of phenyl (6-(1-(2,2-difluorocyclopropyl)-3-methyl-1H-pyrazol-4-yl)-5-((S)-1-hydroxyethyl)-[2,3′-bipyridin]-6′-yl)carbamate

To a stirred solution of (1S)-1-(6′-amino-6-(1-(2,2-difluorocyclopropyl)-3-methyl-1H-pyrazol-4-yl)-[2,3′-bipyridin]-5-yl)ethan-1-ol (obtained as described in step 1 of I-164 synthesis) (300 mg, 0.808 mmol) and 2,4,6-trimethylpyridine (147 mg, 1.21 mmol) in THF (5 mL) was added phenyl carbonochloridate (164 mg, 1.05 mmol) dropwise at 0° C. under N₂ atmosphere. The resulting mixture was stirred at room temperature for 3 h under N₂ atmosphere and then concentrated under reduced pressure. The residue was purified by Prep-TLC (EtOAc) to afford the title compound. LCMS: 492 [M+H]⁺.

### Step 6. Synthesis of 1-(6-(1-(2,2-difluorocyclopropyl)-3-methyl-1H-pyrazol-4-yl)-5-((S)-1-hydroxyethyl)-[2,3′-bipyridin]-6′-yl)-3-((R)-1-(5-(difluoromethyl)pyrazin-2-yl)ethyl)urea

To a stirred solution of phenyl (6-(1-(2,2-difluorocyclopropyl)-3-methyl-1H-pyrazol-4-yl)-5-((S)-1-hydroxyethyl)-[2,3′-bipyridin]-6′-yl)carbamate (120 mg, 0.244 mmol) and (R)-1-(5-(difluoromethyl)pyrazin-2-yl)ethan-1-amine (127 mg, 0.732 mmol) in CH₂Cl₂ (5 mL) was added Et₃N (1 mL) at 0° C. under N₂ atmosphere. The resulting mixture was stirred at 40° C. for 2 hours. The reaction mixture was cooled down to room temperature and then concentrated under reduced pressure. The residue was purified by Prep-TLC (CH₂Cl₂/MeOH) followed by reverse phase prep-HPLC (H₂O (10 mmol/L NH₄HCO₃)-ACN) to provide the title compound. LCMS: 571 [M+H]⁺.

### Step 7. Chiral separation of 1-(6-(1-(2,2-difluorocyclopropyl)-3-methyl-1H-pyrazol-4-yl)-5-((S)-1-hydroxyethyl)-[2,3′-bipyridin]-6′-yl)-3-((R)-1-(5-(difluoromethyl)pyrazin-2-yl)ethyl)urea

The mixture of stereoisomers of 1-(6-(1-(2,2-difluorocyclopropyl)-3-methyl-1H-pyrazol-4-yl)-5-((S)-1-hydroxyethyl)-[2,3′-bipyridin]-6′-yl)-3-((R)-1-(5-(difluoromethyl)pyrazin-2-yl)ethyl)urea was separated by Chiral-HPLC with the following conditions (Column: (R, R)-Whelk-O1, 5 m, 21.1 mm×250 mm; Mobile Phase A: MeOH: CH₂Cl₂=1:1, Mobile Phase B: Hex (with 0.5% NH₃ (2 M in MeOH)); Flow rate: 20 mL/min; Gradient (B %) 70% B; Wave Length: 220/254 nm; Retention time: Peak1: 13.05 min, Peak2: 15.65 min) to afford the title compounds.

Peak 1 (first eluting): 1-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-3-[(1R)-1-[5-(difluoromethyl)pyrazin-2-yl]ethyl]urea (stereoisomer 1) (I-318). ¹H NMR (400 MHz, DMSO-d₆) δ 9.50 (s, 1H), 8.95 (s, 2H), 8.86 (s, 1H), 8.79 (s, 1H), 8.38-8.36 (m, 1H), 8.05-8.01 (m, 2H), 7.93-7.91 (m, 1H), 7.50 (d, J=8.8 Hz, 1H), 7.13 (t, J=54.2 Hz, 1H), 5.26 (d, J=4.4 Hz, 1H), 5.19-5.12 (m, 1H), 4.89-4.85 (m, 1H), 4.57-4.51 (m, 1H), 2.48-2.42 (m, 1H), 2.38-2.30 (m, 1H), 2.28 (s, 3H), 1.52 (d, J=7.0 Hz, 3H), 1.34 (d, J=6.4 Hz, 3H). LCMS: 571.2 [M+H]⁺. Analytical chiral HPLC: Rt=3.403 min.

Peak 2 (second eluting): 1-(6-{1-[(1R*)-2,2-difluorocyclopropyl]-3-methyl-1H-pyrazol-4-yl}-5-[(1S)-1-hydroxyethyl]-[2,3′-bipyridin]-6′-yl)-3-[(1R)-1-[5-(difluoromethyl)pyrazin-2-yl]ethyl]urea (stereoisomer 2) (I-319). ¹H NMR (400 MHz, DMSO-d₆) δ 9.50 (s, 1H), 8.95 (s, 2H), 8.86 (s, 1H), 8.79 (s, 1H), 8.38-8.36 (m, 1H), 8.05-8.01 (m, 2H), 7.93-7.91 (m, 1H), 7.50 (d, J=8.8 Hz, 1H), 7.13 (t, J=54.2 Hz, 1H), 5.26 (d, J=4.4 Hz, 1H), 5.19-5.12 (m, 1H), 4.89-4.85 (m, 1H), 4.57-4.51 (m, 1H), 2.48-2.42 (m, 1H), 2.38-2.30 (m, 1H), 2.28 (s, 3H), 1.52 (d, J=7.0 Hz, 3H), 1.34 (d, J=6.4 Hz, 3H). LCMS: 571.2 [M+H]⁺. Analytical chiral HPLC: Rt=3.899 min.

Analytical chiral HPLC method: Column: CHIRALPAK IA-3; Mobile Phase A: Hexane (0.1% DEA): (EtOH:CH₂Cl₂=1:1)=75:25; Flow rate: 1 mL/min.

### Example 56. Synthesis of (1S,2R)-2-[5-(difluoromethyl)pyrimidin-2-yl]-N-{6-[1-(2-fluoro-2-methylpropyl)-3-methyl-1H-pyrazol-4-yl]-5-[(1S)-1-hydroxyethyl]-[2,3′-bipyridin]-6′-yl}cyclopropane-1-carboxamide (I-224)

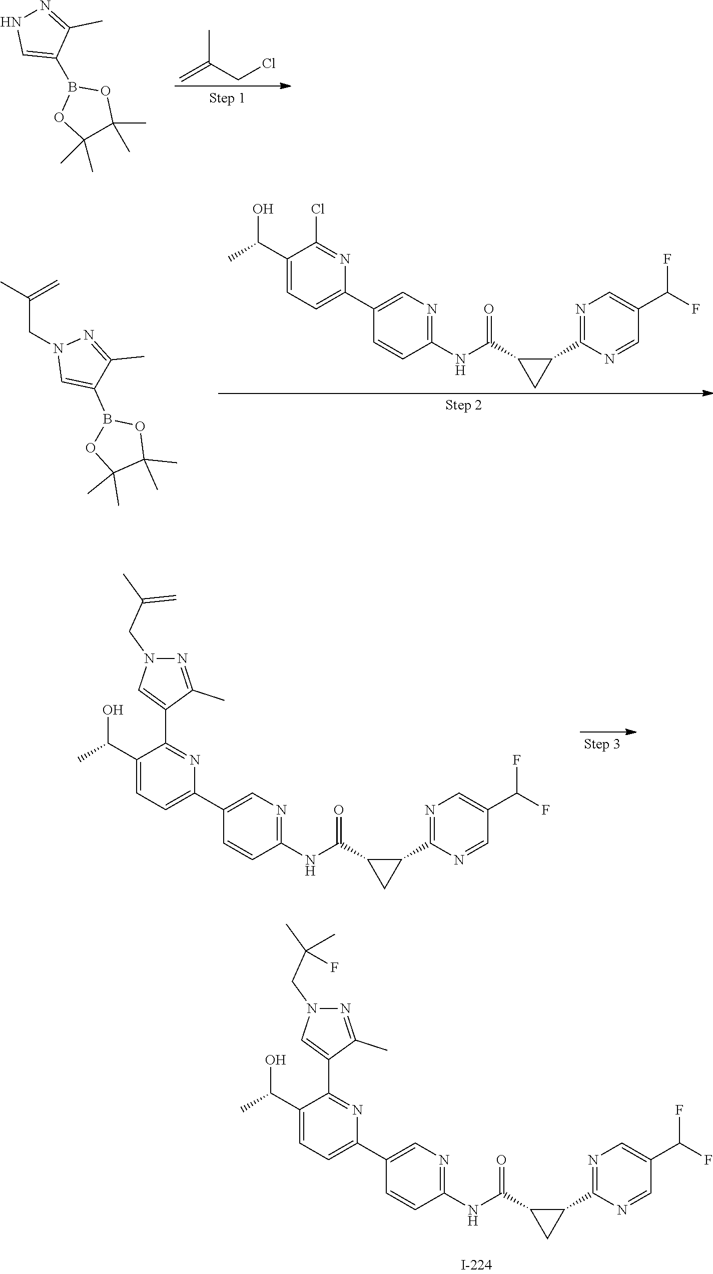

### Step 1. Synthesis of 3-methyl-1-(2-methylallyl)-4-(4,4,5,5-tetramethyl-1,3,2-dioxaborolan-2-yl)-1H-pyrazole

To a solution of 3-methyl-4-(4,4,5,5-tetramethyl-1,3,2-dioxaborolan-2-yl)-1H-pyrazole (1.00 g, 4.81 mmol, 1.0 eq) in ACN (20 mL) were added K₂CO₃ (1.99 g, 14.43 mmol, 3.0 eq) and 3-chloro-2-methylprop-1-ene (870 mg, 9.61 mmol, 2.0 eq) at room temperature. The resulting mixture was stirred at 100° C. for 16 h under N₂ atmosphere. The reaction mixture was filtered and the filter cake was washed with ACN (5 mL*3). The combined filtrate was concentrated under reduced pressure and the residue was purified by chiral SFC (SFC Preparative Method: Instrument: Waters 150 Preparative SFC system; Column: CHIRALART Cellulose-SC, 250 mm×30 mm, 5 m; Mobile Phase A: CO₂, Mobile Phase B: IPA (with 0.3% NH₃ (7 M in MeOH)); Isocratic elution: 12% Phase B in Supercritical CO₂; Flow rate: 70 mL/min; Retention time: Peak 1: 3.03 min; Peak 2: 4.23 min; Back Pressure: 100 bar to keep the CO₂ in Supercritical flow; Wave Length: 220 nm) to afford the title compound as first eluting peak (peak 1) along with its regioisomer (peak 2). LCMS: 263.2 [M+H]⁺.

### Step 2. Synthesis of (1S,2R)-2-(5-(difluoromethyl)pyrimidin-2-yl)-N-(5-((S)-1-hydroxyethyl)-6-(3-methyl-1-(2-methylallyl)-1H-pyrazol-4-yl)-[2,3′-bipyridin]-6′-yl)cyclopropane-1-carboxamide

To a stirred solution of (1S,2R)—N-(6-chloro-5-((S)-1-hydroxyethyl)-[2,3′-bipyridin]-6′-yl)-2-(5-(difluoromethyl)pyrimidin-2-yl)cyclopropane-1-carboxamide (obtained as described in step 4 of I-150 synthesis) (100 mg, 0.22 mmol, 1.0 eq), K₂CO₃ (46 mg, 0.34 mmol, 1.5 eq) and 3-methyl-1-(2-methylallyl)-4-(4,4,5,5-tetramethyl-1,3,2-dioxaborolan-2-yl)-1H-pyrazole (70 mg, 0.27 mmol, 1.2 eq) in dioxane (3 mL) and H₂O (0.30 mL) was added Pd(PPh₃)₄(26 mg, 0.02 mmol, 0.1 eq) at room temperature under N₂ atmosphere. The resulting mixture was stirred at 80° C. for 16 h under N₂ atmosphere. The reaction mixture was cooled down to room temperature and concentrated under reduced pressure. The residue was purified by flash silica gel chromatography (Eluent of MeOH/DCM) to afford the title compound. LCMS: 546.2 [M+H]⁺.

### Step 3. Synthesis of (1S,2R)-2-[5-(difluoromethyl)pyrimidin-2-yl]-N-{6-[1-(2-fluoro-2-methylpropyl)-3-methyl-1H-pyrazol-4-yl]-5-[(1S)-1-hydroxyethyl]-[2,3′-bipyridin]-6′-yl}cyclopropane-1-carboxamide

To a stirred solution of ferric nitrate nonahydrate (178 mg, 1.08 mmol, 4.0 eq) in H₂O (6.00 mL) were added ACN (6 mL) and SelectFlour (156 mg, 1.08 mmol, 4.0 eq) at 0° C. under N₂ atmosphere. To the above mixture were added (1S,2R)-2-(5-(difluoromethyl)pyrimidin-2-yl)-N-(5-((S)-1-hydroxyethyl)-6-(3-methyl-1-(2-methylallyl)-1H-pyrazol-4-yl)-[2,3′-bipyridin]-6′-yl)cyclopropane-1-carboxamide (60 mg, 0.27 mmol, 1.0 eq) and NaBH₄ (27 mg, 1.76 mmol, 6.5 eq). The reaction mixture was stirred at 0° C. for 2 minutes. Another portion of NaBH₄ (27 mg, 1.76 mmol, 6.5 eq) was added and the reaction mixture was stirred at 0° C. for 30 minutes. The reaction mixture was quenched with ice-water (1 mL) at 0° C., diluted with water (20 mL) and extracted with DCM:MeOH (10:1 v/v) (10 mL*3). The combined organic layers were dried over Na₂SO₄, filtered and the filtrate was concentrated under reduced pressure. The residue was dissolved in DMF and purified by reverse-phase prep-HPLC (H₂O (0.1% FA-ACN)) to afford the title compound. ¹H NMR (400 MHz, DMSO-d₆) δ=10.96 (s, 1H), 9.08-8.96 (m, 1H), 8.96-8.80 (m, 2H), 8.40-8.24 (m, 11H), 8.02 (d, 11H), 7.97-7.77 (m, 3H), 7.40-6.89 (m, 11H), 5.26 (s, 1H), 5.01-4.71 (m, 1H), 4.31 (d, 2H), 2.98-2.80 (m, 1H), 2.68-2.52 (m, 1H), 2.28 (s, 3H), 1.98-1.85 (m, 1H), 1.55-1.42 (m, 1H), 1.42-1.21 (m, 9H). LCMS: 566.2 [M+H]⁺.

### Example 57. Synthesis of (1S,2R)-2-(5-{bicyclo[1.1.1]pentan-1-yl}pyrimidin-2-yl)-N-{5-[(1S)-1-hydroxyethyl]-6-{[(3S)-1-methylpyrrolidin-3-yl]oxy}-[2,3′-bipyridin]-6′-yl}cyclopropane-1-carboxamide (I-240)

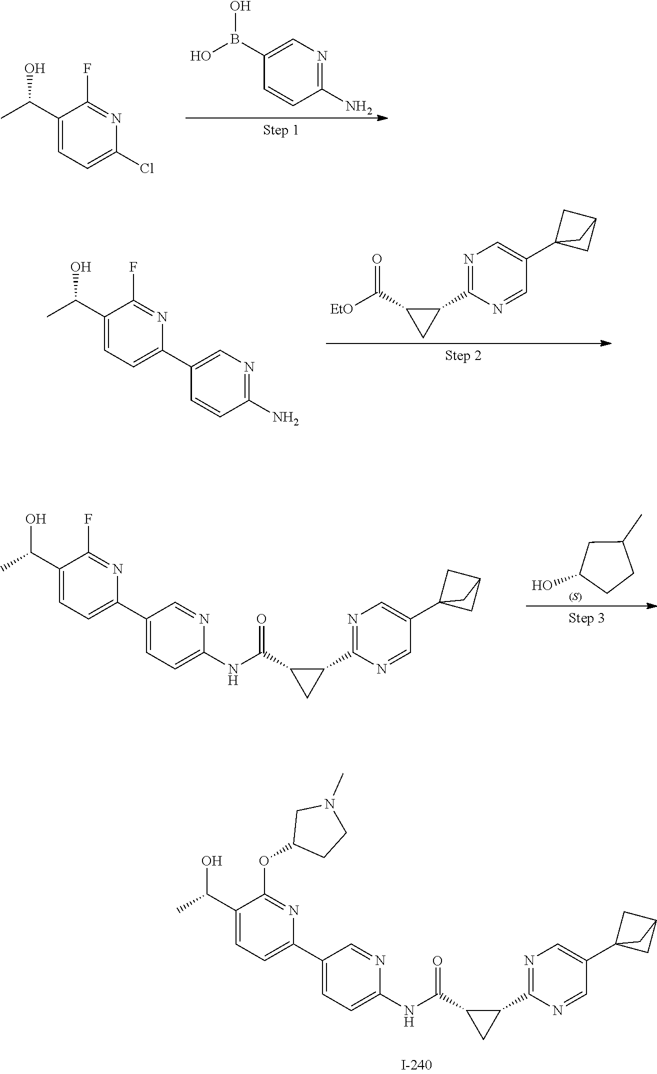

### Step 1. Synthesis of (S)-1-(6′-amino-6-fluoro-[2,3′-bipyridin]-5-yl)ethan-1-ol

To a stirred solution of (S)-1-(6-chloro-2-fluoropyridin-3-yl)ethan-1-ol (1.05 g, 5.98 mmol, 1.0 eq), (6-aminopyridin-3-yl)boronic acid (0.91 g, 6.58 mmol, 1.1 eq.) and K₃PO₄ (5.85 g, 17.94 mmol, 3.0 eq) in dioxane (20 mL) and H₂O (2 mL) was added Pd(dppf)Cl₂*CH₂Cl₂ (134 mg, 0.59 mmol, 0.1 eq) at room temperature under N₂ atmosphere. The resulting mixture was stirred at 80° C. for 1 h under N₂ atmosphere. The reaction mixture was cooled down to room temperature and concentrated under reduced pressure. The residue was purified by flash silica gel chromatography (Eluent of DCM/MeOH) to afford the title compound. LCMS: 234.1 [M+H]⁺.

### Step 2. Synthesis of (1S,2R)-2-(5-(bicyclo[1.1.1]pentan-1-yl)pyrimidin-2-yl)-N-(6-fluoro-5-((S)-1-hydroxyethyl)-[2,3′-bipyridin]-6′-yl)cyclopropane-1-carboxamide

To a stirred solution of (S)-1-(6′-amino-6-fluoro-[2,3′-bipyridin]-5-yl)ethan-1-ol (100 mg, 0.43 mmol, 1.0 eq) and ethyl (1S,2R)-2-(5-(bicyclo[1.1.1]pentan-1-yl)pyrimidin-2-yl)cyclopropane-1-carboxylate (obtained as described in step 3 of I-152 synthesis) (133 mg, 0.52 mmol, 1.2 eq) in THF (1 mL) was added trimethylaluminium (0.65 mL, 2.0 M in toluene, 3.0 eq) in portions at 0° C. under N₂ atmosphere. The resulting mixture was stirred at 80° C. for 1 h, cooled to room temperature and concentrated under reduced pressure. The residue was purified by Prep-TLC (Eluent of EtOAc/Petroleum ether) to afford the title compound. LCMS: 446.2 [M+H]⁺.

### Step 3 Synthesis of (1S,2R)-2-(5-{bicyclo[1.1.1]pentan-1-yl}pyrimidin-2-yl)-N-{5-[(1S)-1-hydroxyethyl]-6-{[(3S)-1-methylpyrrolidin-3-yl]oxy}-[2,3′-bipyridin]-6′-yl}cyclopropane-1-carboxamide

To a solution of (1S,2R)-2-(5-(bicyclo[1.1.1]pentan-1-yl)pyrimidin-2-yl)-N-(6-fluoro-5-((S)-1-hydroxyethyl)-[2,3′-bipyridin]-6′-yl)cyclopropane-1-carboxamide (80 mg, 0.18 mmol, 1.0 eq) and Cs₂CO₃ (176 mg, 0.54 mmol, 3.0 eq) in DMF (7.00 mL) was added (S)-1-methylpyrrolidin-3-ol (36 mg, 0.36 mmol, 2.0 eq) at room temperature. The resulting mixture was stirred at 60° C. for 12 h. The reaction mixture was diluted with H₂O (15.0 mL) and extracted with EtOAc (3 mL*3). The combined organic layers were dried over Na₂SO₄, filtered, and the filtrate was concentrated under reduced pressure. The residue was purified by Prep-HPLC (H₂O (10 mmol/L NH₄HCO₃+0.05% NH₃H₂O)-ACN) to afford the title compound. ¹H NMR (400 MHz, DMSO-d₆) δ 10.89 (s, 1H), 8.95 (s, 1H), 8.52 (s, 2H), 8.30-8.23 (m, 1H), 7.87-7.81 (m, 2H), 7.56-7.54 (d, 1H), 5.51-5.49 (m, 1H), 5.18 (d, 1H), 4.89-4.87 (m, 1H), 2.94-2.90 (m, 1H), 2.79-2.77 (m, 1H), 2.68-2.62 (m, 1H), 2.58 (s, 3H), 2.56-2.55 (m, 1H), 2.51-2.50 (m, 1H), 2.43 (s, 3H), 2.33 (s, 6H), 1.87-1.83 (m, 2H), 1.42-1.39 (m, 1H), 1.31 (d, 3H). LCMS: 527.3 [M+H]⁺.

### Example 58. Synthesis of N-{6-[1-(2-fluoro-2-methylpropyl)-3-methyl-1H-pyrazol-4-yl]-5-[(1S)-1-hydroxyethyl]-[2,3′-bipyridin]-6′-yl}-2-{5H,6H,8H-imidazo [2,1-c][1,4]oxazin-2-yl}acetamide (I-304) and N-{6-[1-(2-fluoro-2-methylpropyl)-5-methyl-1H-pyrazol-4-yl]-5-[(1S)-1-hydroxyethyl]-[2,3′-bipyridin]-6′-yl}-2-{5H,6H,8H-imidazo [2,1-c][1,4]oxazin-2-yl}acetamide (I-305)

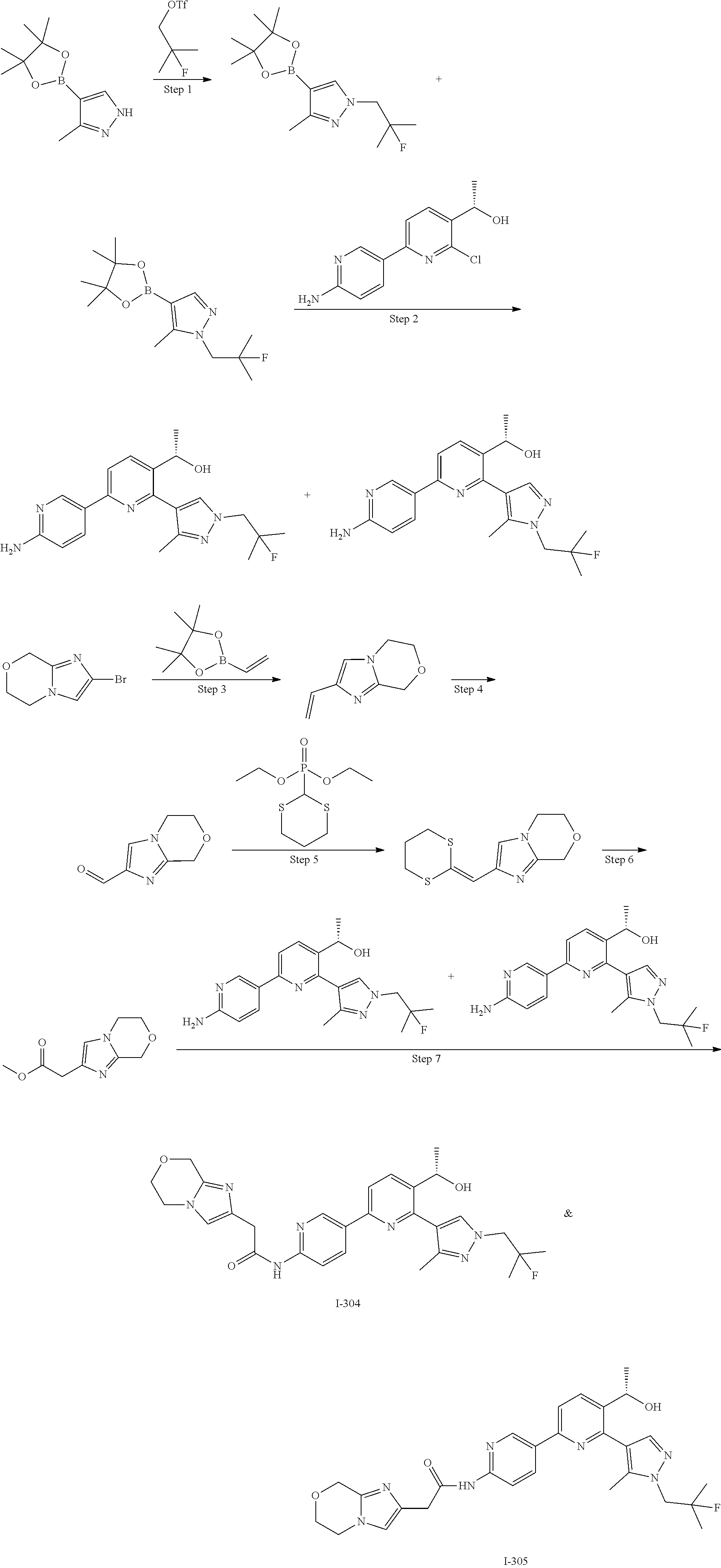

### Step 1. Synthesis of 1-(2-fluoro-2-methylpropyl)-3-methyl-4-(4,4,5,5-tetramethyl-1,3,2-dioxaborolan-2-yl)-1H-pyrazole and 1-(2-fluoro-2-methylpropyl)-5-methyl-4-(4,4,5,5-tetramethyl-1,3,2-dioxaborolan-2-yl)-1H-pyrazole

To a stirred solution of 3-methyl-4-(4,4,5,5-tetramethyl-1,3,2-dioxaborolan-2-yl)-1H-pyrazole (4.00 g, 19.23 mmol, 1.0 eq) and Cs₂CO₃ (18.7 g, 57.69 mmol, 3.0 eq) in MeCN (40.00 mL) was added 2-fluoro-2-methylpropyl trifluoromethanesulfonate (12.9 g, 57.69 mmol, 3.0 eq) at room temperature. The resulting mixture was stirred at room temperature overnight. The reaction mixture was concentrated under reduced pressure. The residue was purified by flash silica gel chromatography (Eluent of PE/EtOAc) to afford a mixture of the title compounds (ratio ~2:1). LCMS: 283.2 [M+H]⁺

### Step 2. Synthesis of (S)-1-(6′-amino-6-(1-(2-fluoro-2-methylpropyl)-3-methyl-1H-pyrazol-4-yl)-[2,3′-bipyridin]-5-yl)ethan-1-ol and (S)-1-(6′-amino-6-(1-(2-fluoro-2-methylpropyl)-5-methyl-1H-pyrazol-4-yl)-[2,3′-bipyridin]-5-yl)ethan-1-ol

A solution of (S)-1-(6′-amino-6-chloro-[2,3′-bipyridin]-5-yl)ethan-1-ol (prepared as described in step 5 of I-152 synthesis) (1.10 g, 4.42 mmol, 1.0 eq), a mixture of 1-(2-fluoro-2-methylpropyl)-3-methyl-4-(4,4,5,5-tetramethyl-1,3,2-dioxaborolan-2-yl)-1H-pyrazole and 1-(2-fluoro-2-methylpropyl)-5-methyl-4-(4,4,5,5-tetramethyl-1,3,2-dioxaborolan-2-yl)-1H-pyrazole from step 1 (2.11 g, 7.51 mmol, 1.7 eq), Pd(PPh₃)₄(510 mg, 0.44 mmol, 0.1 eq) and K₂CO₃(1.83 g, 13.26 mmol, 3.0 eq) in 1,4-dioxane (16 mL) and H₂O (2 mL) was stirred at 100° C. for 1 h under N₂ atmosphere. The reaction mixture was allowed to cool down to room temperature, diluted with water (80 mL) and extracted with EtOAc (3*60 mL). The combined organic phase was dried over Na₂SO₄, filtered, and the filtrate was concentrated under reduced pressure. The residue was purified by flash silica gel chromatography (eluent of DCM/MeOH) to afford a mixture of the title compounds (ratio of ~1.5:1). LCMS: 370.2 [M+H]⁺.

### Step 3. Synthesis of 2-vinyl-5,6-dihydro-8H-imidazo[2,1-c][1,4]oxazine

To a solution of 2-bromo-5,6-dihydro-8H-imidazo[2,1-c][1,4]oxazine (2 g, 9.85 mmol) in 1,4-dioxane (30 mL) and H₂O (3 mL) were added 4,4,5,5-tetramethyl-2-vinyl-1,3,2-dioxaborolane (6.83 g, 44.3 mmol), K₃PO₄ (7.32 g, 34.4 mmol) and Pd(dppf)Cl₂*CH₂Cl₂ (1.21 g, 1.48 mmol) and the resulting mixture was stirred overnight at 80° C. under N₂ atmosphere. The reaction mixture was cooled down to room temperature, diluted with water (60 mL) and extracted with CH₂Cl₂ (60 mL*3). The combined organic phases were dried over anhydrous Na₂SO₄, filtered, and the filtrate was concentrated under reduced pressure. The residue was purified using prep-TLC (Eluent of CH₂Cl₂/MeOH) to afford the title compound. LCMS: 151 [M+H]⁺.

### Step 4. Synthesis of 5,6-dihydro-8H-imidazo[2,1-c][1,4]oxazine-2-carbaldehyde

To a solution of potassium osmate(VI) dihydrate (288 mg, 0.78 mmol) and NaIO₄ (6.68 g, 31.2 mmol) in THF (12 mL) and H₂O (12 mL) was added 2-vinyl-5,6-dihydro-8H-imidazo[2,1-c][1,4]oxazine (1.26 g, 7.80 mmol) in THF (12 mL) at 0° C. The resulting mixture was stirred at room temperature for 1 h. The reaction mixture was poured into sat. aq. Na₂S₂O₃ (80 mL) and extracted with CH₂Cl₂/MeOH (10:1 v/v) (50 mL*5). The combined organic layers were dried over anhydrous Na₂SO₄, filtered, and the filtrate was concentrated under reduced pressure. The residue was purified by prep-TLC (Eluent of CH₂Cl₂/MeOH) to afford the title compound. LCMS: 153 [M+H]⁺.

### Step 5. Synthesis of 2-((1,3-dithian-2-ylidene)methyl)-5,6-dihydro-8H-imidazo[2,1-c][1,4]oxazine

To a stirred solution of diethyl (1,3-dithian-2-yl)phosphonate (1.62 g, 6.31 mmol) in THF (7 mL) was added n-BuLi (3.37 mL, 8.41 mmol, 2.5 M in n-hexane) at 0° C. under N₂ atmosphere. The resulting mixture was stirred at 0° C. for 10 minutes. To the above reaction mixture was slowly added 5,6-dihydro-8H-imidazo[2,1-c][1,4]oxazine-2-carbaldehyde (800 mg, 5.26 mmol) in THF (3 mL) dropwise at −78° C. and the resulting mixture was stirred at room temperature for 1 h. The reaction mixture was quenched with sat. aq. NH₄Cl (30 mL) and extracted with EtOAc (35 mL*3). The combined organic layers were washed with brine, dried over anhydrous Na₂SO₄, filtered and the filtrate was concentrated under reduced pressure to provide the title compound which was used in the next step directly without further purification. LCMS: 255 [M+H]⁺.

### Step 6. Synthesis of methyl 2-(5,6-dihydro-8H-imidazo[2,1-c][1,4]oxazin-2-yl)acetate

To a stirred solution of 2-((1,3-dithian-2-ylidene)methyl)-5,6-dihydro-8H-imidazo[2,1-c][1,4]oxazine (1 g, 3.93 mmol) in MeOH (10 mL) was added a solution of HCl in MeOH (4.0 M 5.9 mL, 23.6 mmol) at room temperature and the resulting mixture was stirred overnight at 60° C. The reaction mixture was cooled down to room temperature and concentrated under reduced pressure. The residue was poured into sat. aq. NaHCO₃ (60 mL) and extracted with CH₂Cl₂/MeOH (10:1 v/v) (50 mL*3). The combined organic layers were dried over anhydrous Na₂SO₄, filtered and the filtrate was concentrated under reduced pressure. The residue was purified using reverse phase flash chromatography (C18 silica gel, 15% to 20% MeCN in water (10 mmol/L NH₄HCO₃)) to provide the title compound. LCMS: 197 [M+H]⁺.

### Step 7. Synthesis of N-{6-[1-(2-fluoro-2-methylpropyl)-3-methyl-1H-pyrazol-4-yl]-5-[(1S)-1-hydroxyethyl]-[2,3′-bipyridin]-6′-yl}-2-{5H,6H,8H-imidazo[2,1-c][1,4]oxazin-2-yl}acetamide and N-{6-[1-(2-fluoro-2-methylpropyl)-5-methyl-1H-pyrazol-4-yl]-5-[(1S)-1-hydroxyethyl]-[2,3′-bipyridin]-6′-yl}-2-{5H,6H,8H-imidazo[2,1-c][1,4]oxazin-2-yl}acetamide

To a stirred solution of a mixture of (S)-1-(6′-amino-6-(1-(2-fluoro-2-methylpropyl)-3-methyl-1H-pyrazol-4-yl)-[2,3′-bipyridin]-5-yl)ethan-1-ol and (S)-1-(6′-amino-6-(1-(2-fluoro-2-methylpropyl)-5-methyl-1H-pyrazol-4-yl)-[2,3′-bipyridin]-5-yl)ethan-1-ol (prepared in step 2) (240 mg, 0.65 mmol) and methyl 2-(5,6-dihydro-8H-imidazo[2,1-c][1,4]oxazin-2-yl)acetate (357 mg, 1.82 mmol) in toluene (12 mL) was added LiHMDS (2.6 mL, 2.60 mmol, 1.0 M in toluene) at 0° C. under N₂ atmosphere. The resulting mixture was stirred at 60° C. for 3 h under N₂ atmosphere. The reaction mixture was cooled down to room temperature, quenched with ice-water and concentrated under reduced pressure. The residue was purified using prep-TLC (CH₂Cl₂/MeOH) to afford the mixture of title isomers which was separated by chiral-HPLC (Column: CHIRALPAK IM, 5 m, 20 mm*250 mm; Mobile Phase A: EtOH:CH₂Cl₂=1:1, Mobile Phase B: Hexane (with 0.5% NH₃(2 M in MeOH)); Flow rate: 20 mL/min; Gradient (B %): 40% B; Wave Length: 220/254 nm; Rt (peak1)=4.91 min; Rt (peak2)=6.18 min) to afford the title compounds.

N-{6-[1-(2-fluoro-2-methylpropyl)-3-methyl-1H-pyrazol-4-yl]-5-[(1S)-1-hydroxyethyl]-[2,3′-bipyridin]-6′-yl}-2-{5H,6H,8H-imidazo[2,1-c][1,4]oxazin-2-yl}acetamide (I-304): ¹H NMR (400 MHz, DMSO-d6) δ 10.65 (s, 1H), 9.03 (d, J=2.4 Hz, 1H), 8.47-8.43 (m, 1H), 8.19 (d, J=8.7 Hz, 1H), 8.05 (d, J=8.3 Hz, 1H), 7.93 (d, J=8.2 Hz, 1H), 7.84 (s, 1H), 7.02 (s, 1H), 5.27 (d, J=4.4 Hz, 1H), 4.95-4.88 (m, 1H), 4.70 (s, 2H), 4.35 (s, 1H), 4.30 (s, 1H), 3.99 (s, 4H), 3.63 (s, 2H), 2.31 (s, 3H), 1.39-1.29 (m, 9H). LCMS: 534 [M+H]⁺. Analytical chiral HPLC: Rt=1.197 min.

N-{6-[1-(2-fluoro-2-methylpropyl)-5-methyl-IH-pyrazol-4-yl]-5-[(1S)-1-hydroxyethyl]-[2,3′-bipyridin]-6′-yl}-2-{5H,6H,8H-imidazo[2,1-c][1,4]oxazin-2-yl}acetamide (I-305): ¹H NMR (400 MHz, DMSO-d₆) δ 10.65 (s, 1H), 9.01 (d, J=2.5 Hz, 1H), 8.45-8.41 (m, 1H), 8.19 (d, J=8.8 Hz, 1H), 8.06 (d, J=8.1 Hz, 1H), 7.94 (d, J=8.2 Hz, 1H), 7.64 (s, 1H), 7.02 (s, 1H), 5.27 (d, J=4.3 Hz, 1H), 4.96-4.89 (m, 1H), 4.70 (s, 2H), 4.39 (s, 1H), 4.34 (s, 1H), 3.98 (s, 4H), 3.62 (s, 2H), 2.37 (s, 3H), 1.42-1.30 (m, 9H). LCMS: 534 [M+H]⁺. Analytical chiral HPLC: Rt=1.558 min.

Analytical chiral HPLC method: Column: CHIRALPAK IM-3; Mobile Phase A: Hexane (0.1% DEA): (EtOH:CH₂Cl₂=1:1)=40:60; Flow rate: 1 mL/min.

### Example 59. Synthesis of N-{6-[1-(2-fluoro-2-methylpropyl)-3-methyl-1H-pyrazol-4-yl]-5-[(1S)-1-hydroxyethyl]-[2,3′-bipyridin]-6′-yl}-2-(5-methylpyridin-2-yl)acetamide (I-281) and N—{6-[1-(2-fluoro-2-methylpropyl)-5-methyl-1H-pyrazol-4-yl]-5-[(1S)-1-hydroxyethyl]-[2,3′-bipyridin]-6′-yl}-2-(5-methylpyridin-2-yl)acetamide (I-282)

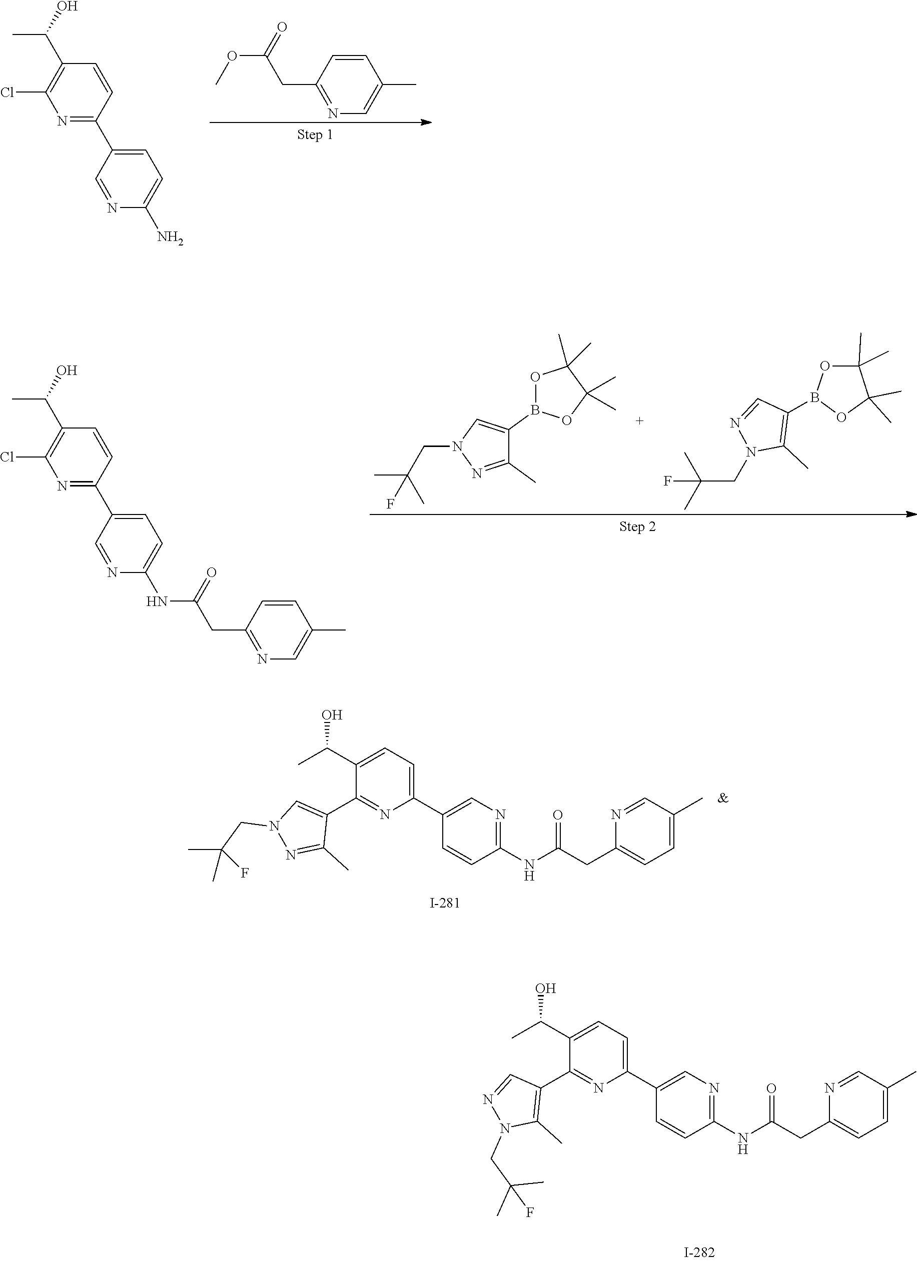

### Step 1. Synthesis of (S)—N-(6-chloro-5-(1-hydroxyethyl)-[2,3′-bipyridin]-6′-yl)-2-(5-methylpyridin-2-yl)acetamide

To a solution of (S)-1-(6′-amino-6-chloro-[2,3′-bipyridin]-5-yl)ethan-1-ol (prepared as described in step 5 of I-152 synthesis) (500 mg, 2.00 mmol, 1.0 eq) and methyl 2-(5-methylpyridin-2-yl)acetate (496 mg, 3.00 mmol, 1.5 eq) in toluene (8.00 mL) was added LiHMDS (6.00 mL, 1.0 M in THF, 3.0 eq) at 0° C. under N₂ atmosphere. The resulting mixture was stirred at room temperature for 3 h under N₂ atmosphere. The reaction mixture was diluted with ice-water (50 mL) and extracted with EtOAc (3*30 mL). The combined organic phase was dried over Na₂SO₄, filtered and the filtrate was concentrated under reduced pressure. The residue was purified by Prep-TLC (Eluent of MeOH/DCM) to provide the title compound. LCMS: 383.1 [M+H]⁺.

### Step 2. Synthesis of N-{6-[1-(2-fluoro-2-methylpropyl)-3-methyl-1H-pyrazol-4-yl]-5-[(1S)-1-hydroxyethyl]-[2,3′-bipyridin]-6′-yl}-2-(5-methylpyridin-2-yl)acetamide and N-{6-[1-(2-fluoro-2-methylpropyl)-5-methyl-1H-pyrazol-4-yl]-5-[(1S)-1-hydroxyethyl]-[2,3′-bipyridin]-6′-yl}-2-(5-methylpyridin-2-yl)acetamide

To a stirred solution of (S)—N-(6-chloro-5-(1-hydroxyethyl)-[2,3′-bipyridin]-6′-yl)-2-(5-methylpyridin-2-yl)acetamide (100 mg, 0.261 mmol), a mixture of 1-(2-fluoro-2-methylpropyl)-3-methyl-4-(4,4,5,5-tetramethyl-1,3,2-dioxaborolan-2-yl)-1H-pyrazole and 1-(2-fluoro-2-methylpropyl)-5-methyl-4-(4,4,5,5-tetramethyl-1,3,2-dioxaborolan-2-yl)-1H-pyrazole (prepared as described in step 1 of I-304 synthesis) (221 mg, 0.783 mmol) and K₂CO₃ (108 mg, 0.783 mmol) in dioxane (2 mL) and H₂O (0.2 mL) was added Pd(PPh₃)₄(30.1 mg, 0.026 mmol) at room temperature under N₂ atmosphere. The resulting mixture was stirred at 100° C. for 1 h under N₂ atmosphere. The reaction mixture was allowed to cool down to room temperature, diluted with water (30 mL) and extracted with EtOAc (50 mL*3). The combined organic layers were washed with brine, dried over anhydrous Na₂SO₄, filtered and the filtrate was concentrated under reduced pressure. The residue was purified using prep-TLC (Eluent of CH₂Cl₂/MeOH) to afford the mixture of title isomers which was separated by preparative chiral HPLC (Column: CHIRALPAK® IM, 5 m, 20 mm 250 mm; Mobile Phase A: EtOH: CH₂Cl₂=1:1, Mobile Phase B: Hex (with 0.5% NH₃ (2 M in MeOH)); Flow rate: 20 mL/min; Gradient (B %) 55% B; Wave Length: 220/254 nm; R^{†}(peak1)=5.58 min; R^{†}(peak2)=8.28; Sample solvent: EtOH: CH₂Cl₂=1:1) to provide the title compounds.

N-{6-[1-(2-fluoro-2-methylpropyl)-3-methyl-1H-pyrazol-4-yl]-5-[(1S)-1-hydroxyethyl]-[2,3′-bipyridin]-6′-yl}-2-(5-methylpyridin-2-yl)acetamide (I-281): ¹H NMR (400 MHz, DMSO-d₆) δ 10.88 (s, 1H), 9.07-9.02 (m, 1H), 8.49-8.42 (m, 1H), 8.36 (d, J=2.3 Hz, 1H), 8.17 (d, J=8.8 Hz, 1H), 8.05 (d, J=8.2 Hz, 1H), 7.93 (d, J=8.2 Hz, 1H), 7.85 (s, 1H), 7.63-7.56 (m, 1H), 7.31 (d, J=7.9 Hz, 1H), 5.28 (d, J=4.4 Hz, 1H), 4.96-4.86 (m, 1H), 4.35 (s, 1H), 4.30 (s, 1H), 3.93 (s, 2H), 2.31 (s, 3H), 2.29 (s, 3H), 1.37 (d, J=6.4 Hz, 3H), 1.32 (t, J=6.4 Hz, 6H). LCMS: 503 [M+H]⁺. Analytical chiral HPLC: Rt=1.66 min.

N-{6-[1-(2-fluoro-2-methylpropyl)-5-methyl-1H-pyrazol-4-yl]-5-[(1S)-1-hydroxyethyl]-[2,3′-bipyridin]-6′-yl}-2-(5-methylpyridin-2-yl)acetamide (I-282): ¹H NMR (400 MHz, DMSO-d₆) δ 10.88 (s, 1H), 9.03 (d, J=2.4 Hz, 1H), 8.47-8.40 (m, 1H), 8.35 (d, J=2.3 Hz, 1H), 8.17 (d, J=8.7 Hz, 1H), 8.06 (d, J=8.2 Hz, 1H), 7.94 (d, J=8.2 Hz, 1H), 7.64 (s, 1H), 7.62-7.55 (m, 1H), 7.31 (d, J=7.9 Hz, 1H), 5.27 (d, J=4.3 Hz, 1H), 4.97-4.86 (m, 1H), 4.39 (s, 1H), 4.34 (s, 1H), 3.92 (s, 2H), 2.37 (s, 3H), 2.28 (s, 3H), 1.41 (d, J=1.9 Hz, 3H), 1.36 (d, J=1.9 Hz, 3H), 1.32 (d, J=6.3 Hz, 3H). LCMS: 503 [M+H]⁺. Analytical chiral HPLC: Rt=3.07 min.

Analytical chiral HPLC method: Column: CHIRALPAK IM3; Mobile Phase A: Hexane (0.1% DEA): (EtOH:CH₂Cl₂=1:1)=60:40; Flow rate: 1 mL/min.

### Example 60. Synthesis of (1S,2R)—N-(6-{1-[(1R)-2,2-difluorocyclopropyl]-3-methyl-1H-pyrazol-4-yl}-5-[(1S)-1-hydroxyethyl]-[2,3′-bipyridin]-6′-yl)-2-[5-(trifluoromethyl)pyrimidin-2-yl]cyclopropane-1-carboxamide (I-226)

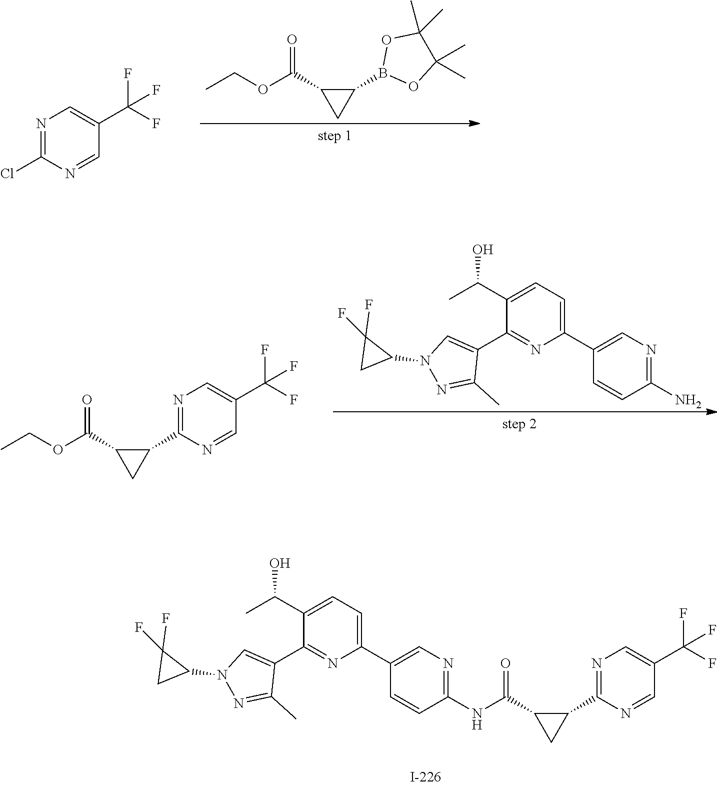

### Step 1. Synthesis of ethyl (1S,2R)-2-(5-(trifluoromethyl)pyrimidin-2-yl)cyclopropane-1-carboxylate

To a solution of 2-chloro-5-(trifluoromethyl)pyrimidine (1.00 g, 5.48 mmol, 1.0 eq) and ethyl (1S,2R)-2-(4,4,5,5-tetramethyl-1,3,2-dioxaborolan-2-yl)cyclopropane-1-carboxylate (1.45 g, 6.03 mmol, 1.1 eq) in 1,4-dioxane (6 mL) and H₂O (1.2 mL) was added Pd(OAc)₂ (246 mg, 1.10 mmol, 0.2 eq), di(adamantan-1-yl)(butyl)phosphane (589 mg, 1.64 mmol, 0.3 eq) and Cs₂CO₃ (5.36 g, 16.44 mmol, 3.0 eq), then the resulting mixture was stirred at 100° C. overnight under N₂ atmosphere. The reaction mixture was poured into water (50 mL), extracted with EtOAc (30 mL*2). The combined organic layers were washed with brine (30 mL), dried over anhydrous Na₂SO₄, filtered and concentrated under reduced pressure. The residue was purified by flash silica gel chromatography (Eluent of EtOAc/PE) and then preparative normal phase HPLC (Eluent of Heptane-EtOH) to afford the title compound.

LCMS: 261.1 [M+H]⁺

### Step 2. Synthesis of (1S,2R)—N-(6-{1-[(1R)-2,2-difluorocyclopropyl]-3-methyl-1H-pyrazol-4-yl}-5-[(1S)-1-hydroxyethyl]-[2,3′-bipyridin]-6′-yl)-2-[5-(trifluoromethyl)pyrimidin-2-yl]cyclopropane-1-carboxamide

To AlMe₃ (2.0 M in toluene, 1.01 mL, 5.0 eq) was added dropwise a solution of ethyl (1S,2R)-2-(5-(trifluoromethyl)pyrimidin-2-yl)cyclopropane-1-carboxylate (126 mg, 484 mol, 1.2 eq) and (S)-1-(6′-amino-6-(1-((R)-2,2-difluorocyclopropyl)-3-methyl-1H-pyrazol-4-yl)-[2,3′-bipyridin]-5-yl)ethan-1-ol (150 mg, 404 mol, 1.0 eq) in 1,4-dioxane (2 mL) at 90° C. under N₂ atmosphere and the resulting mixture was stirred at 90° C. for 3 hours. The reaction mixture was cooled to 0° C., quenched with sat. aq. potassium sodium tartrate solution (10 mL) under N₂, then diluted with H₂O (10 mL) and extracted with EtOAc (3*20 mL). The combined organic layers were washed with brine (30 mL), dried over anhydrous Na₂SO₄, filtered and the filtrate was concentrated under reduced pressure. The residue was purified by flash silica gel chromatography (Eluent of EtOAc/PE) to afford the title compound. LCMS: 586.2 [M+H]⁺. ¹H NMR (400 MHz, DMSO-d₆) δ 10.98 (s, 1H), 9.13 (s, 2H), 8.98 (d, 1H), 8.37-8.28 (m, 1H), 8.08-7.99 (m, 2H), 7.94-7.81 (m, 2H), 5.25 (d, 1H), 4.92-4.78 (m, 1H), 4.60-4.46 (m, 1H), 3.00-2.90 (m, 1H), 2.69-2.60 (m, 1H), 2.46-2.38 (m, 1H), 2.36-2.28 (m, 1H), 2.24 (s, 3H), 1.95-1.87 (m, 1H), 1.56-1.47 (m, 1H), 1.30 (d, 3H).

TABLE 2
Selected Compounds

| | | | Mass | Re- |
|---|---|---|---|---|
| | | | Ob- | tention |
| | | LCMS | served | time |
| I-# | Structure | Method | [g/mol] | [min] |
| I-1 | 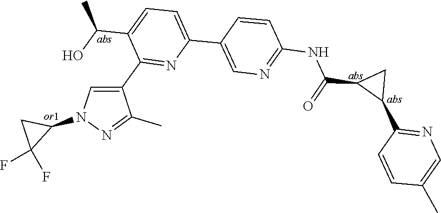 [SMILES: Cc1ccc([C@@H]2C[C@@H]2C(=O)Nc2ccc(-c3ccc([C@H](C)O)c(-c4cn([C@@H]5CC5(F)F)nc4C)n3)cn2)nc1] | LCMS 4 | 531.3 | 0.404 |
| I-2 | 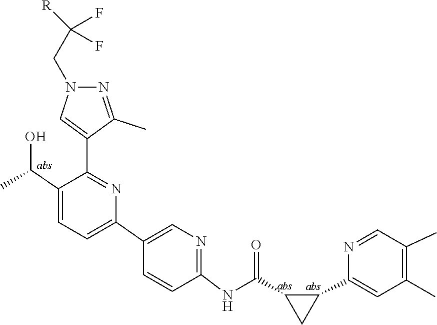 | LCMS 2 | 551.25 | 1.36 |
| I-3 | 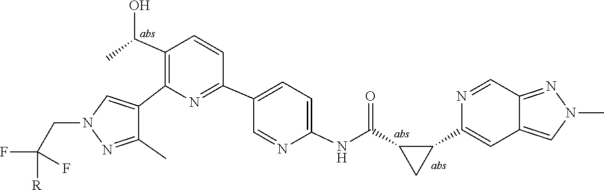 | LCMS 4 | 577.3 | 0.412 |
| I-4 | 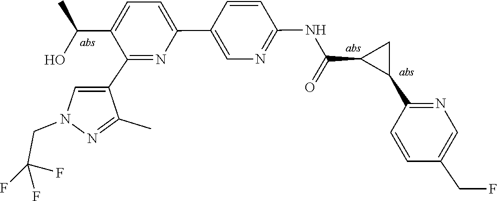 [SMILES: Cc1nn(CC(F)(F)F)cc1-c1nc(-c2ccc(NC(=O)[C@H]3C[C@H]3c3ccc(CF)cn3)nc2)ccc1[C@H](C)O] | LCMS 5 | 555.3 | 0.55 |
| I-5 | 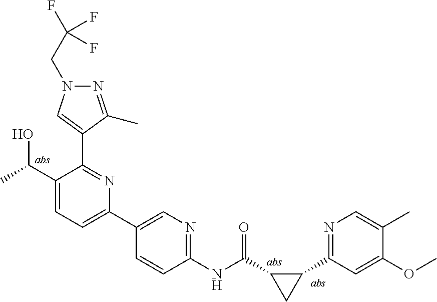 [SMILES: COc1cc([C@@H]2C[C@@H]2C(=O)Nc2ccc(-c3ccc([C@H](C)O)c(-c4cn(CC(F)(F)F)nc4C)n3)cn2)ncc1C] | LCMS 2 | 567.25 | 1.39 |
| I-6 | 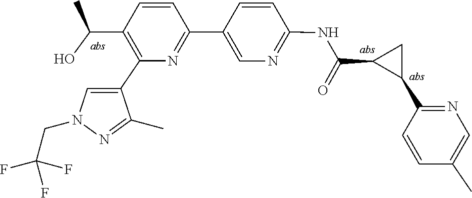 [SMILES: Cc1ccc([C@@H]2C[C@@H]2C(=O)Nc2ccc(-c3ccc([C@H](C)O)c(-c4cn(CC(F)(F)F)nc4C)n3)cn2)nc1] | LCMS 4 | 537.3 | 0.416 |
| I-7 | 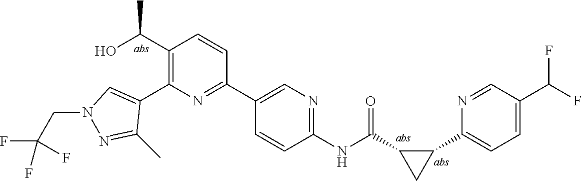 [SMILES: Cc1nn(CC(F)(F)F)cc1-c1nc(-c2ccc(NC(=O)[C@H]3C[C@H]3c3ccc(C(F)F)cn3)nc2)ccc1[C@H](C)O] | LCMS 4 | 573.3 | 0.463 |
| I-8 | 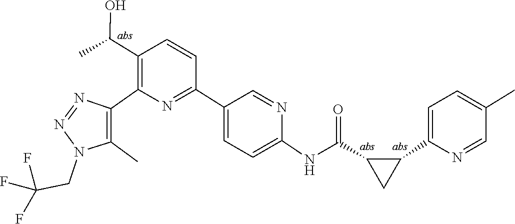 [SMILES: Cc1ccc([C@@H]2C[C@@H]2C(=O)Nc2ccc(-c3ccc([C@H](C)O)c(-c4nnn(CC(F)(F)F)c4C)n3)cn2)nc1] | LCMS 4 | 538.3 | 0.435 |
| I-9 | 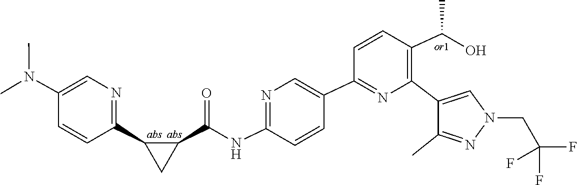 [SMILES: Cc1nn(CC(F)(F)F)cc1-c1nc(-c2ccc(NC(=O)[C@H]3C[C@H]3c3ccc(N(C)C)cn3)nc2)ccc1[C@H](C)O] | LCMS 2 | 566.25 | 1.26 |
| I-10 | 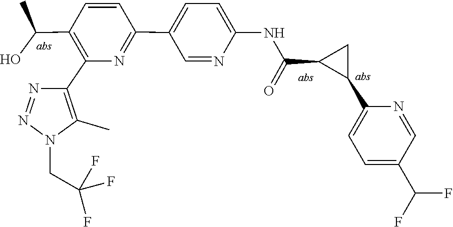 [SMILES: Cc1c(-c2nc(-c3ccc(NC(=O)[C@H]4C[C@H]4c4ccc(C(F)F)cn4)nc3)ccc2[C@H](C)O)nnn1CC(F)(F)F] | LCMS 4 | 574.3 | 0.489 |
| I-11 | 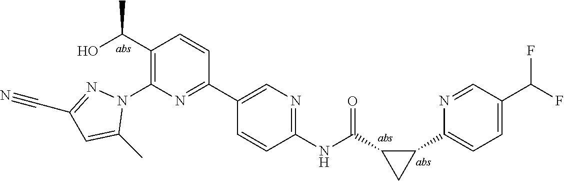 [SMILES: Cc1cc(C#N)nn1-c1nc(-c2ccc(NC(=O)[C@H]3C[C@H]3c3ccc(C(F)F)cn3)nc2)ccc1[C@H](C)O] | LCMS 5 | 516.3 | 0.619 |
| I-12 | 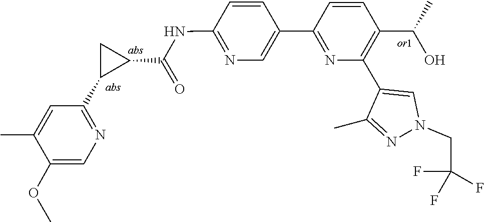 [SMILES: COc1cnc([C@@H]2C[C@@H]2C(=O)Nc2ccc(-c3ccc([C@H](C)O)c(-c4cn(CC(F)(F)F)nc4C)n3)cn2)cc1C] | LCMS 2 | 567.25 | 1.34 |
| I-13 | 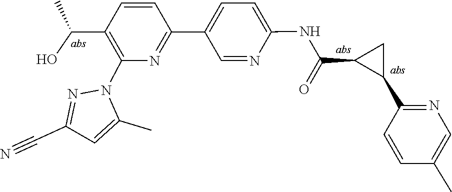 [SMILES: Cc1ccc([C@@H]2C[C@@H]2C(=O)Nc2ccc(-c3ccc([C@@H](C)O)c(-n4nc(C#N)cc4C)n3)cn2)nc1] | LCMS 5 | 480.3 | 0.583 |
| I-14 | 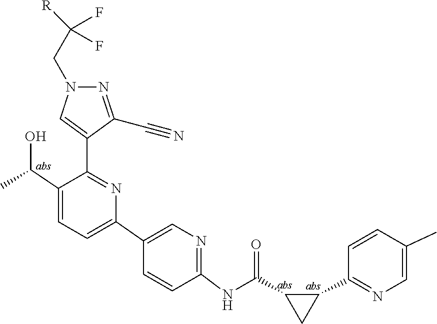 | LCMS 2 | 548.2 | 1.52 |
| I-15 | 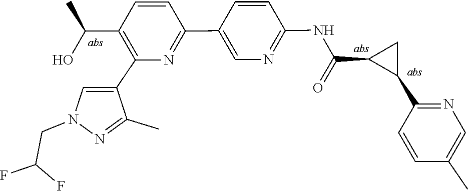 [SMILES: Cc1ccc([C@@H]2C[C@@H]2C(=O)Nc2ccc(-c3ccc([C@H](C)O)c(-c4cn(CC(F)F)nc4C)n3)cn2)nc1] | LCMS 4 | 519.1 | 0.402 |
| I-16 | 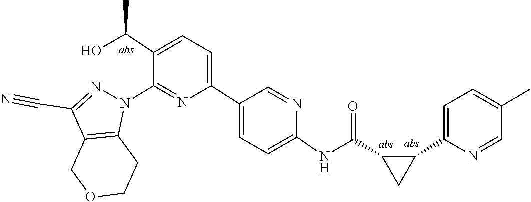 [SMILES: Cc1ccc([C@@H]2C[C@@H]2C(=O)Nc2ccc(-c3ccc([C@H](C)O)c(-n4nc(C#N)c5c4CCOC5)n3)cn2)nc1] | LCMS 4 | 522.3 | 0.43 |
| I-17 | 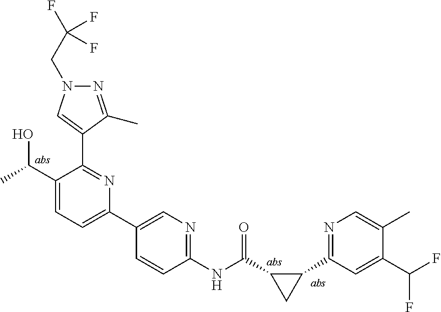 [SMILES: Cc1cnc([C@@H]2C[C@@H]2C(=O)Nc2ccc(-c3ccc([C@H](C)O)c(-c4cn(CC(F)(F)F)nc4C)n3)cn2)cc1C(F)F] | LCMS 2 | 587.2 | 1.48 |
| I-18 | 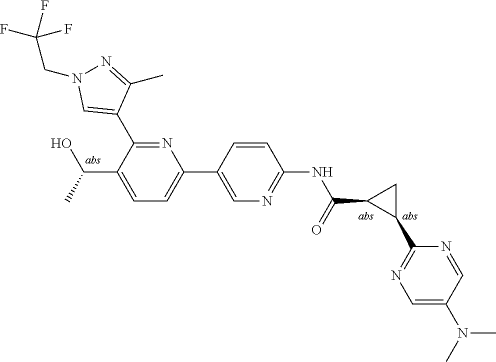 [SMILES: Cc1nn(CC(F)(F)F)cc1-c1nc(-c2ccc(NC(=O)[C@H]3C[C@H]3c3ncc(N(C)C)cn3)nc2)ccc1[C@H](C)O] | LCMS 1 | 567.25 | 1.08 |
| I-19 | 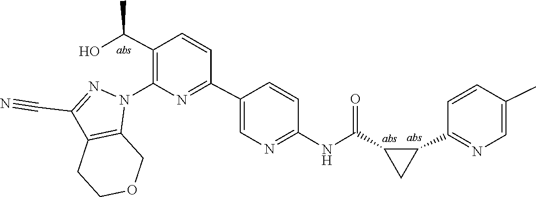 [SMILES: Cc1ccc([C@@H]2C[C@@H]2C(=O)Nc2ccc(-c3ccc([C@H](C)O)c(-n4nc(C#N)c5c4COCC5)n3)cn2)nc1] | LCMS 5 | 522.3 | 0.637 |
| I-20 | 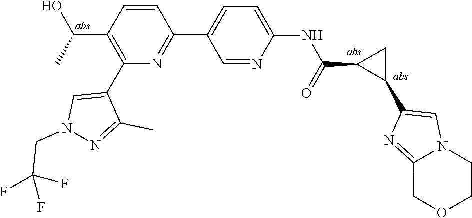 [SMILES: Cc1nn(CC(F)(F)F)cc1-c1nc(-c2ccc(NC(=O)[C@H]3C[C@H]3c3cn4c(n3)COCC4)nc2)ccc1[C@H](C)O] | LCMS 4 | 568.4 | 0.406 |
| I-21 | 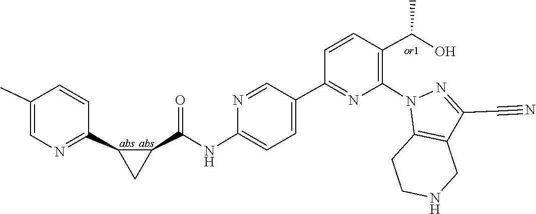 [SMILES: Cc1ccc([C@@H]2C[C@@H]2C(=O)Nc2ccc(-c3ccc([C@H](C)O)c(-n4nc(C#N)c5c4CCNC5)n3)cn2)nc1] | LCMS 2 | 521.2 | 1.15 |
| I-22 | 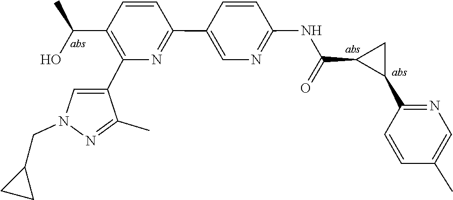 [SMILES: Cc1ccc([C@@H]2C[C@@H]2C(=O)Nc2ccc(-c3ccc([C@H](C)O)c(-c4cn(CC5CC5)nc4C)n3)cn2)nc1] | LCMS 4 | 509.3 | 0.41 |
| I-23 | 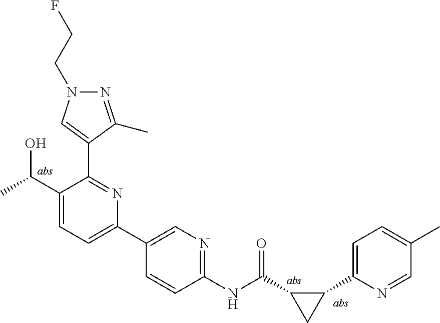 [SMILES: Cc1ccc([C@@H]2C[C@@H]2C(=O)Nc2ccc(-c3ccc([C@H](C)O)c(-c4cn(CCF)nc4C)n3)cn2)nc1] | LCMS 2 | 501.2 | 1.01 |
| I-24 | 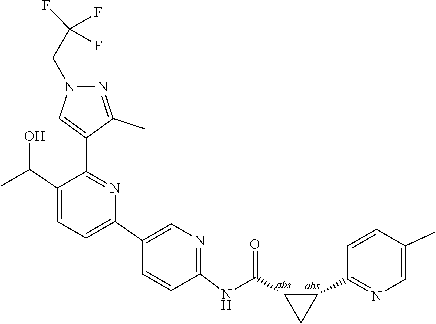 [SMILES: Cc1ccc([C@@H]2C[C@@H]2C(=O)Nc2ccc(-c3ccc(C(C)O)c(-c4cn(CC(F)(F)F)nc4C)n3)cn2)nc1] | LCMS 4 | 537.2 | 0.402 |
| I-25 | 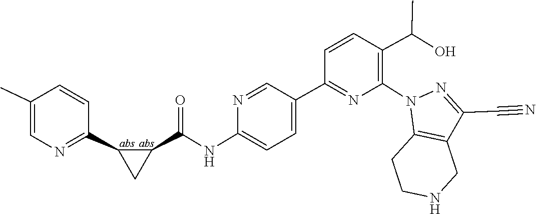 [SMILES: Cc1ccc([C@@H]2C[C@@H]2C(=O)Nc2ccc(-c3ccc(C(C)O)c(-n4nc(C#N)c5c4CCNC5)n3)cn2)nc1] | LCMS 2 | 521.25 | 1.55 |
| I-26 | 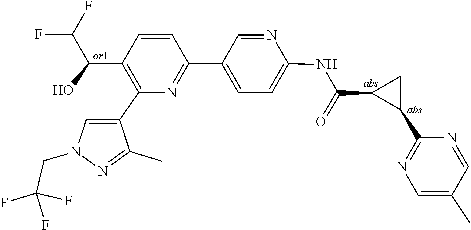 [SMILES: Cc1cnc([C@@H]2C[C@@H]2C(=O)Nc2ccc(-c3ccc([C@@H](O)C(F)F)c(-c4cn(CC(F)(F)F)nc4C)n3)cn2)nc1] | LCMS 5 | 574.3 | 0.554 |
| I-27 | 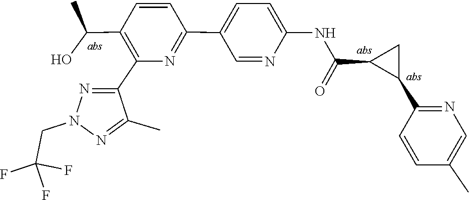 [SMILES: Cc1ccc([C@@H]2C[C@@H]2C(=O)Nc2ccc(-c3ccc([C@H](C)O)c(-c4nn(CC(F)(F)F)nc4C)n3)cn2)nc1] | LCMS 4 | 538.2 | 0.447 |
| I-28 | 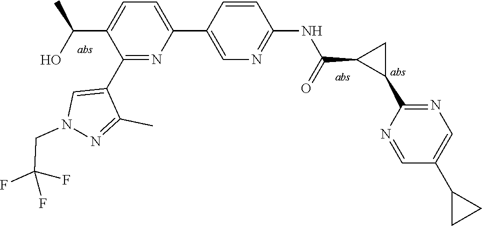 [SMILES: Cc1nn(CC(F)(F)F)cc1-c1nc(-c2ccc(NC(=O)[C@H]3C[C@H]3c3ncc(C4CC4)cn3)nc2)ccc1[C@H](C)O] | LCMS 4 | 564.3 | 0.458 |
| I-29 | 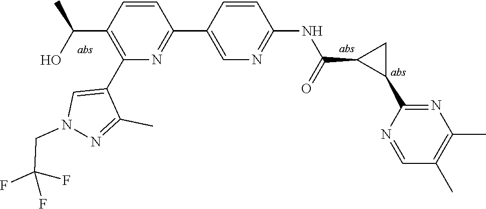 [SMILES: Cc1cnc([C@@H]2C[C@@H]2C(=O)Nc2ccc(-c3ccc([C@H](C)O)c(-c4cn(CC(F)(F)F)nc4C)n3)cn2)nc1C] | LCMS 5 | 552.3 | 0.537 |
| I-30 | 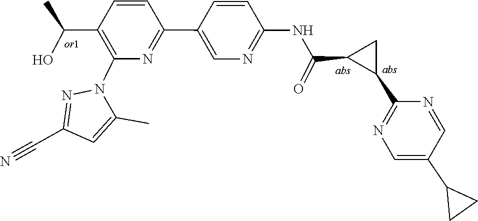 [SMILES: Cc1cc(C#N)nn1-c1nc(-c2ccc(NC(=O)[C@H]3C[C@H]3c3ncc(C4CC4)cn3)nc2)ccc1[C@H](C)O] | LCMS 5 | 507.3 | 0.584 |
| I-31 | 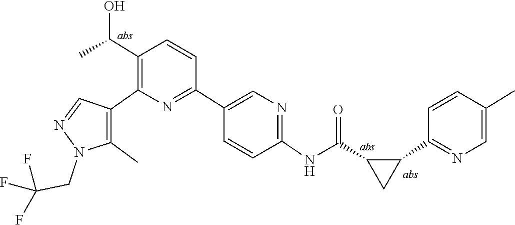 [SMILES: Cc1ccc([C@@H]2C[C@@H]2C(=O)Nc2ccc(-c3ccc([C@H](C)O)c(-c4cnn(CC(F)(F)F)c4C)n3)cn2)nc1] | LCMS 4 | 537.2 | 0.4 |
| I-32 | 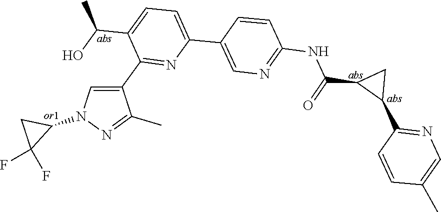 [SMILES: Cc1ccc([C@@H]2C[C@@H]2C(=O)Nc2ccc(-c3ccc([C@H](C)O)c(-c4cn([C@H]5CC5(F)F)nc4C)n3)cn2)nc1] | LCMS 4 | 531.3 | 0.404 |
| I-33 | 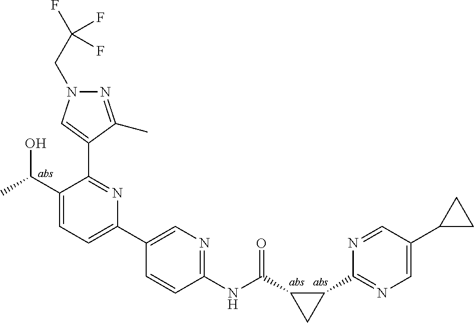 [SMILES: Cc1nn(CC(F)(F)F)cc1-c1nc(-c2ccc(NC(=O)[C@H]3C[C@H]3c3ncc(C4CC4)cn3)nc2)ccc1[C@H](C)O] | LCMS 2 | 564.25 | 1.3 |
| I-34 | 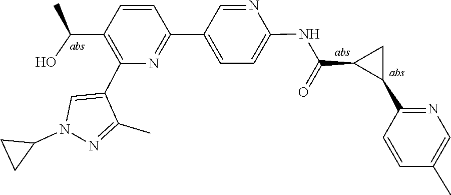 [SMILES: Cc1ccc([C@@H]2C[C@@H]2C(=O)Nc2ccc(-c3ccc([C@H](C)O)c(-c4cn(C5CC5)nc4C)n3)cn2)nc1] | LCMS 5 | 495.3 | 0.537 |
| I-35 | 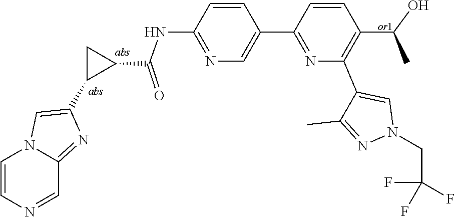 [SMILES: Cc1nn(CC(F)(F)F)cc1-c1nc(-c2ccc(NC(=O)[C@H]3C[C@H]3c3cn4ccncc4n3)nc2)ccc1[C@H](C)O] | LCMS 2 | 563.2 | 1.37 |
| I-36 | 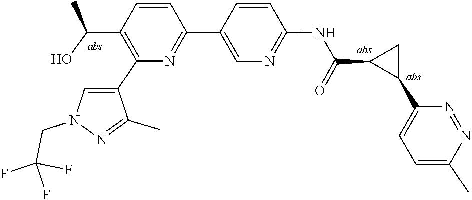 [SMILES: Cc1ccc([C@@H]2C[C@@H]2C(=O)Nc2ccc(-c3ccc([C@H](C)O)c(-c4cn(CC(F)(F)F)nc4C)n3)cn2)nn1] | LCMS 4 | 538.2 | 0.414 |
| I-37 | 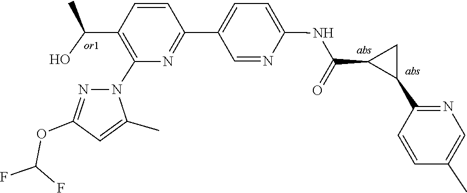 [SMILES: Cc1ccc([C@@H]2C[C@@H]2C(=O)Nc2ccc(-c3ccc([C@H](C)O)c(-n4nc(OC(F)F)cc4C)n3)cn2)nc1] | LCMS 4 | 521.3 | 0.452 |
| I-38 | 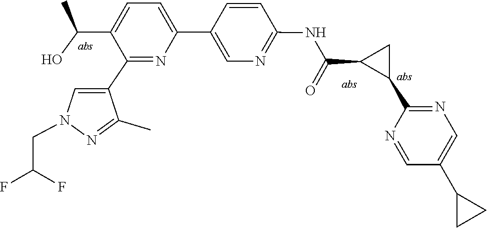 [SMILES: Cc1nn(CC(F)F)cc1-c1nc(-c2ccc(NC(=O)[C@H]3C[C@H]3c3ncc(C4CC4)cn3)nc2)ccc1[C@H](C)O] | LCMS 4 | 546.3 | 0.44 |
| I-39 | 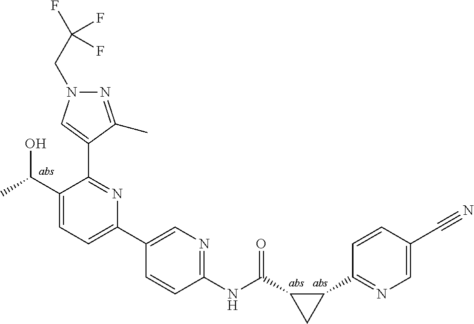 [SMILES: Cc1nn(CC(F)(F)F)cc1-c1nc(-c2ccc(NC(=O)[C@H]3C[C@H]3c3ccc(C#N)cn3)nc2)ccc1[C@H](C)O] | LCMS 2 | 548.25 | 1.17 |
| I-40 | 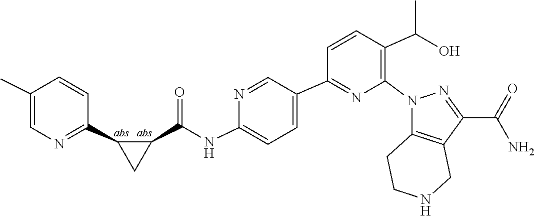 [SMILES: Cc1ccc([C@@H]2C[C@@H]2C(=O)Nc2ccc(-c3ccc(C(C)O)c(-n4nc(C(N)=O)c5c4CCNC5)n3)cn2)nc1] | LCMS 2 | 539.25 | 0.78 |
| I-41 | 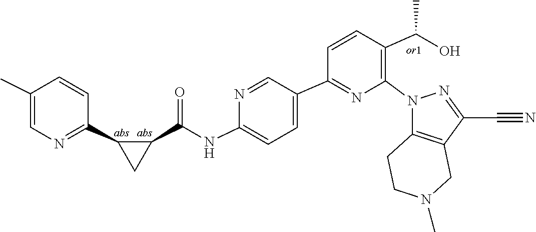 [SMILES: Cc1ccc([C@@H]2C[C@@H]2C(=O)Nc2ccc(-c3ccc([C@H](C)O)c(-n4nc(C#N)c5c4CCN(C)C5)n3)cn2)nc1] | LCMS 2 | 535.2 | 1.26 |
| I-42 | 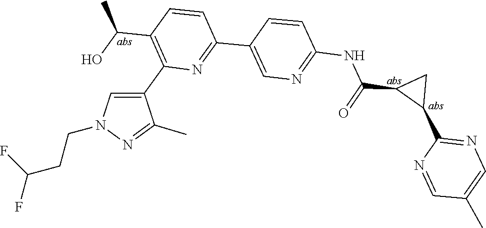 [SMILES: Cc1cnc([C@@H]2C[C@@H]2C(=O)Nc2ccc(-c3ccc([C@H](C)O)c(-c4cn(CCC(F)F)nc4C)n3)cn2)nc1] | LCMS 4 | 534.2 | 0.418 |
| I-43 | 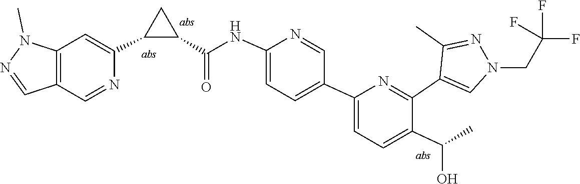 [SMILES: Cc1nn(CC(F)(F)F)cc1-c1nc(-c2ccc(NC(=O)[C@H]3C[C@H]3c3cc4c(cn3)cnn4C)nc2)ccc1[C@H](C)O] | LCMS 2 | 577.2 | 0.9 |
| I-44 | 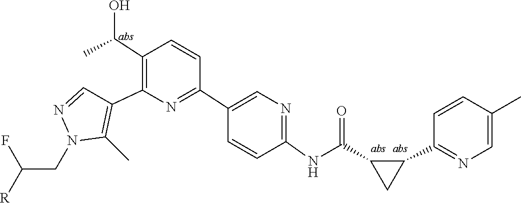 | LCMS 4 | 519.2 | 0.395 |
| I-45 | 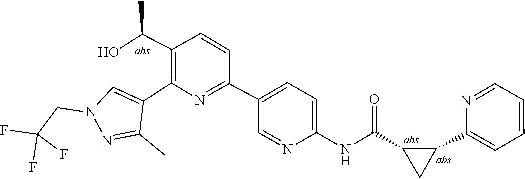 [SMILES: Cc1nn(CC(F)(F)F)cc1-c1nc(-c2ccc(NC(=O)[C@H]3C[C@H]3c3ccccn3)nc2)ccc1[C@H](C)O] | LCMS 5 | 523.3 | 0.543 |
| I-46 | 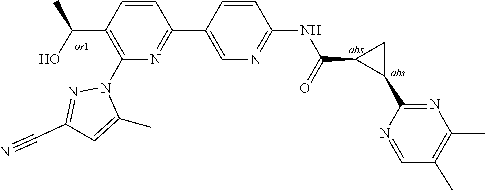 [SMILES: Cc1cnc([C@@H]2C[C@@H]2C(=O)Nc2ccc(-c3ccc([C@H](C)O)c(-n4nc(C#N)cc4C)n3)cn2)nc1C] | LCMS 5 | 495.3 | 0.565 |
| I-47 | 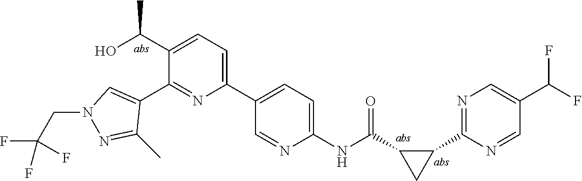 [SMILES: Cc1nn(CC(F)(F)F)cc1-c1nc(-c2ccc(NC(=O)[C@H]3C[C@H]3c3ncc(C(F)F)cn3)nc2)ccc1[C@H](C)O] | LCMS 5 | 574.3 | 0.559 |
| I-48 | 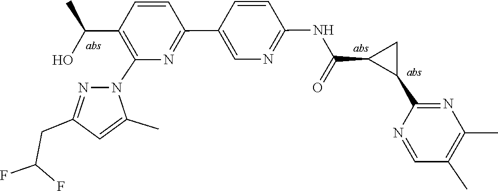 [SMILES: Cc1cnc([C@@H]2C[C@@H]2C(=O)Nc2ccc(-c3ccc([C@H](C)O)c(-n4nc(CC(F)F)cc4C)n3)cn2)nc1C] | LCMS 4 | 534.4 | 0.426 |
| I-49 | 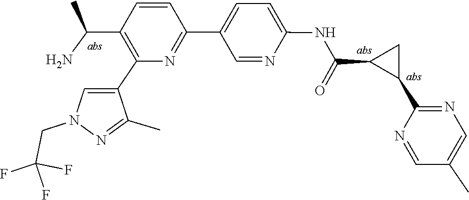 [SMILES: Cc1cnc([C@@H]2C[C@@H]2C(=O)Nc2ccc(-c3ccc([C@H](C)N)c(-c4cn(CC(F)(F)F)nc4C)n3)cn2)nc1] | LCMS 5 | 538.3 | 0.513 |
| I-50 | 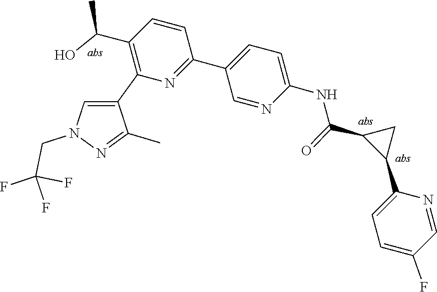 [SMILES: Cc1nn(CC(F)(F)F)cc1-c1nc(-c2ccc(NC(=O)[C@H]3C[C@H]3c3ccc(F)cn3)nc2)ccc1[C@H](C)O] | LCMS 5 | 541.3 | 0.551 |
| I-51 | 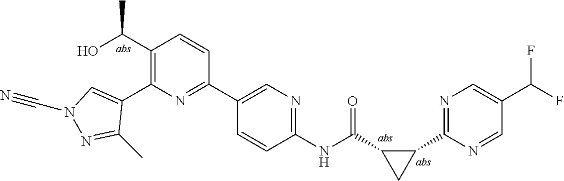 [SMILES: Cc1nn(C#N)cc1-c1nc(-c2ccc(NC(=O)[C@H]3C[C@H]3c3ncc(C(F)F)cn3)nc2)ccc1[C@H](C)O] | LCMS 4 | 517.1 | 0.479 |
| I-52 | 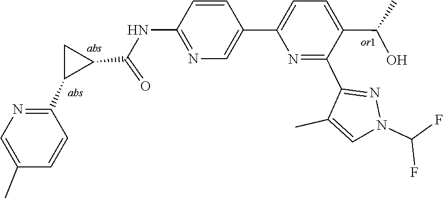 [SMILES: Cc1ccc([C@@H]2C[C@@H]2C(=O)Nc2ccc(-c3ccc([C@H](C)O)c(-c4nn(C(F)F)cc4C)n3)cn2)nc1] | LCMS 2 | 505.15 | 1 |
| I-53 | 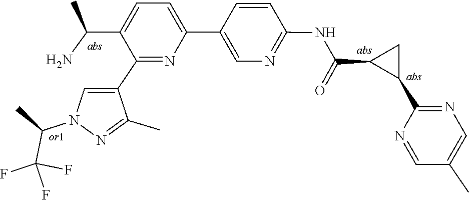 [SMILES: Cc1cnc([C@@H]2C[C@@H]2C(=O)Nc2ccc(-c3ccc([C@H](C)N)c(-c4cn([C@H](C)C(F)(F)F)nc4C)n3)cn2)nc1] | LCMS 5 | 552.3 | 0.557 |
| I-54 | 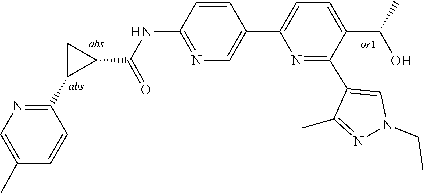 [SMILES: CCn1cc(-c2nc(-c3ccc(NC(=O)[C@H]4C[C@H]4c4ccc(C)cn4)nc3)ccc2[C@H](C)O)c(C)n1] | LCMS 2 | 483.3 | 1.52 |
| I-55 | 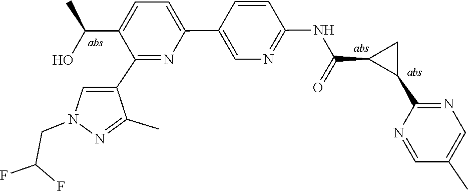 [SMILES: Cc1cnc([C@@H]2C[C@@H]2C(=O)Nc2ccc(-c3ccc([C@H](C)O)c(-c4cn(CC(F)F)nc4C)n3)cn2)nc1] | LCMS 4 | 520.4 | 0.418 |
| I-56 | 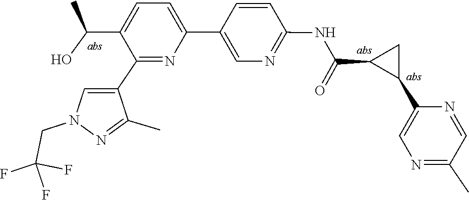 [SMILES: Cc1cnc([C@@H]2C[C@@H]2C(=O)Nc2ccc(-c3ccc([C@H](C)O)c(-c4cn(CC(F)(F)F)nc4C)n3)cn2)cn1] | LCMS 4 | 538.3 | 0.447 |
| I-57 | 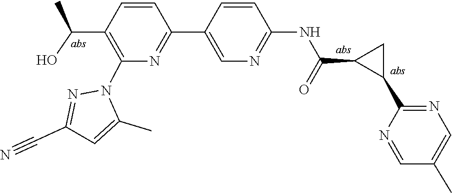 [SMILES: Cc1cnc([C@@H]2C[C@@H]2C(=O)Nc2ccc(-c3ccc([C@H](C)O)c(-n4nc(C#N)cc4C)n3)cn2)nc1] | LCMS 5 | 481.3 | 0.507 |
| I-58 | 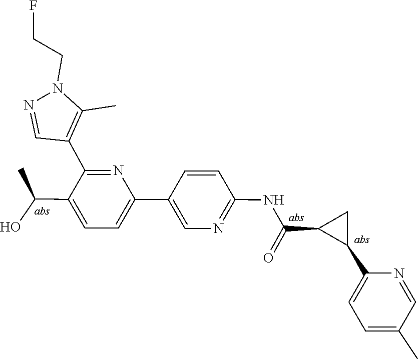 [SMILES: Cc1ccc([C@@H]2C[C@@H]2C(=O)Nc2ccc(-c3ccc([C@H](C)O)c(-c4cnn(CCF)c4C)n3)cn2)nc1] | LCMS 2 | 501.2 | 1.02 |
| I-59 | 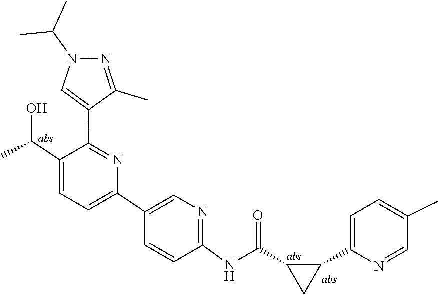 [SMILES: Cc1ccc([C@@H]2C[C@@H]2C(=O)Nc2ccc(-c3ccc([C@H](C)O)c(-c4cn(C(C)C)nc4C)n3)cn2)nc1] | LCMS 2 | 497.2 | 1.21 |
| I-60 | 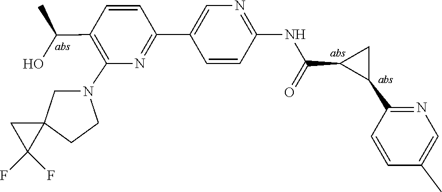 [SMILES: Cc1ccc([C@@H]2C[C@@H]2C(=O)Nc2ccc(-c3ccc([C@H](C)O)c(N4CCC5(C4)CC5(F)F)n3)cn2)nc1] | LCMS 5 | 506.4 | 0.677 |
| I-61 | 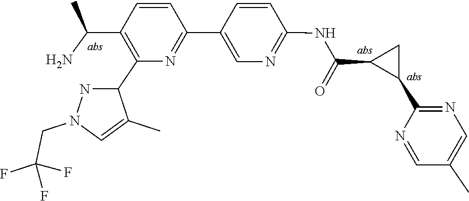 [SMILES: CC1=CN(CC(F)(F)F)NC1c1nc(-c2ccc(NC(=O)[C@H]3C[C@H]3c3ncc(C)cn3)nc2)ccc1[C@H](C)N] | LCMS 4 | 538.2 | 0.471 |
| I-62 | 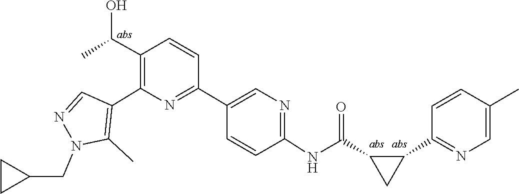 [SMILES: Cc1ccc([C@@H]2C[C@@H]2C(=O)Nc2ccc(-c3ccc([C@H](C)O)c(-c4cnn(CC5CC5)c4C)n3)cn2)nc1] | LCMS 4 | 509.3 | 0.419 |
| I-63 | 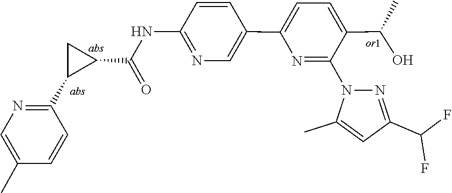 [SMILES: Cc1ccc([C@@H]2C[C@@H]2C(=O)Nc2ccc(-c3ccc([C@H](C)O)c(-n4nc(C(F)F)cc4C)n3)cn2)nc1] | LCMS 2 | 505.2 | 1.19 |
| I-64 | 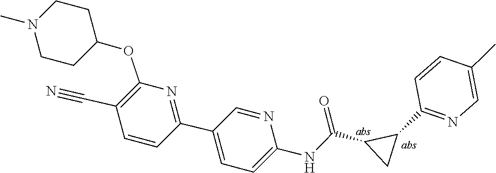 | LCMS 5 | 469.3 | 0.653 |
| I-65 | 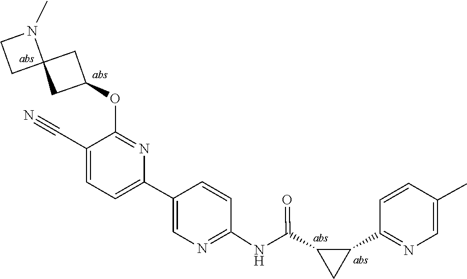 [SMILES: Cc1ccc([C@@H]2C[C@@H]2C(=O)Nc2ccc(-c3ccc(C#N)c(O[C@H]4C[C@]5(CCN5C)C4)n3)cn2)nc1] | LCMS 4 | 481.3 | 0.393 |
| I-66 | 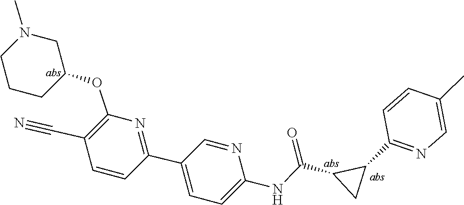 [SMILES: Cc1ccc([C@@H]2C[C@@H]2C(=O)Nc2ccc(-c3ccc(C#N)c(O[C@@H]4CCCN(C)C4)n3)cn2)nc1] | LCMS 2 | 469.15 | 1.57 |
| I-67 | 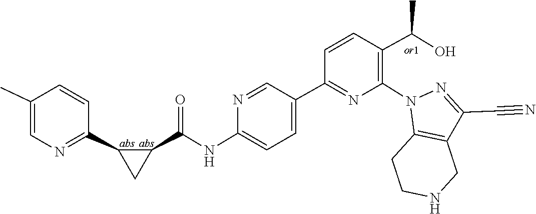 [SMILES: Cc1ccc([C@@H]2C[C@@H]2C(=O)Nc2ccc(-c3ccc([C@@H](C)O)c(-n4nc(C#N)c5c4CCNC5)n3)cn2)nc1] | LCMS 2 | 521.2 | 0.84 |
| I-68 | 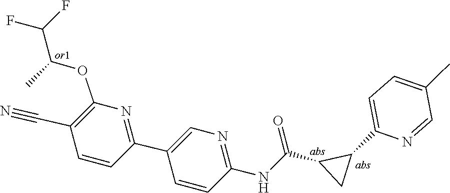 [SMILES: Cc1ccc([C@@H]2C[C@@H]2C(=O)Nc2ccc(-c3ccc(C#N)c(O[C@H](C)C(F)F)n3)cn2)nc1] | LCMS 5 | 450.3 | 0.667 |
| I-69 | 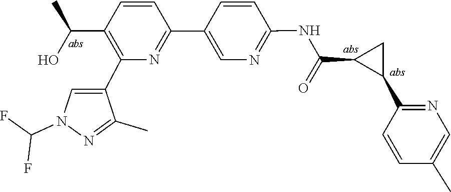 [SMILES: Cc1ccc([C@@H]2C[C@@H]2C(=O)Nc2ccc(-c3ccc([C@H](C)O)c(-c4cn(C(F)F)nc4C)n3)cn2)nc1] | LCMS 4 | 505.2 | 0.405 |
| I-70 | 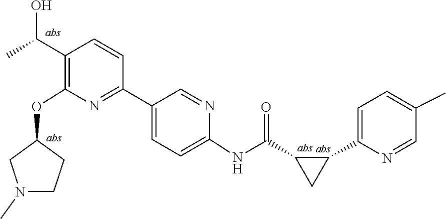 | LCMS 5 | 474.4 | 0.604 |
| I-71 | 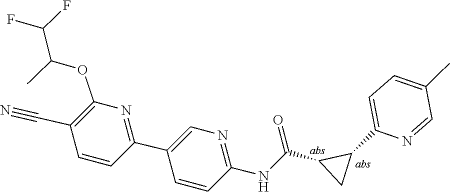 [SMILES: Cc1ccc([C@@H]2C[C@@H]2C(=O)Nc2ccc(-c3ccc(C#N)c(OC(C)C(F)F)n3)cn2)nc1] | LCMS 5 | 450.2 | 0.648 |
| I-72 | 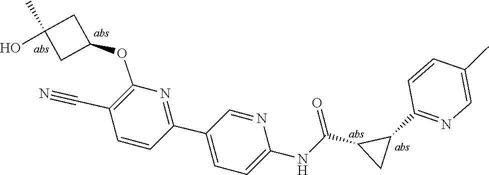 [SMILES: Cc1ccc([C@@H]2C[C@@H]2C(=O)Nc2ccc(-c3ccc(C#N)c(O[C@H]4C[C@@](C)(O)C4)n3)cn2)nc1] | LCMS 5 | 456.3 | 0.578 |
| I-73 | 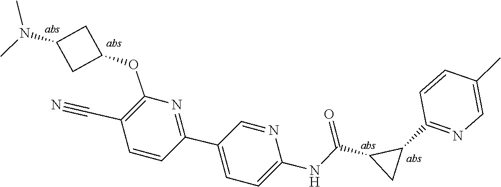 | LCMS 5 | 469.3 | 0.674 |
| I-74 | 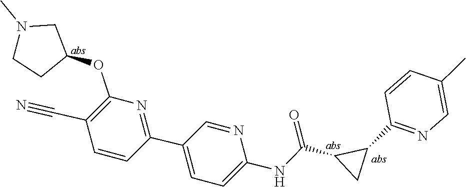 | LCMS 5 | 455.3 | 0.613 |
| I-75 | 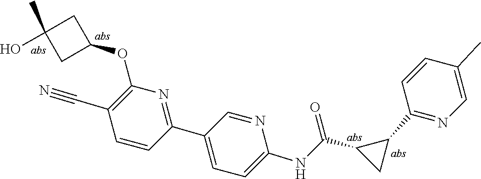 [SMILES: Cc1ccc([C@@H]2C[C@@H]2C(=O)Nc2ccc(-c3ccc(C#N)c(O[C@H]4C[C@](C)(O)C4)n3)cn2)nc1] | LCMS 5 | 456.2 | 0.568 |
| I-76 | 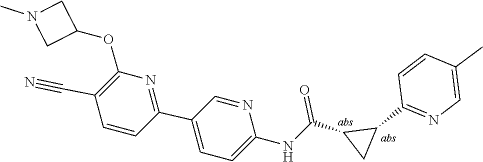 | LCMS 5 | 441.3 | 0.614 |
| I-77 | 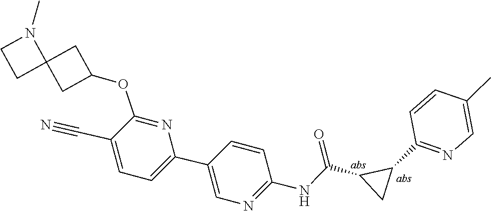 [SMILES: Cc1ccc([C@@H]2C[C@@H]2C(=O)Nc2ccc(-c3ccc(C#N)c(OC4CC5(CCN5C)C4)n3)cn2)nc1] | LCMS 3 | 481.2 | 0.64 |
| I-78 | 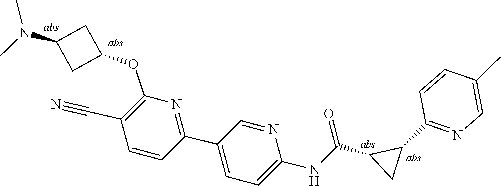 | LCMS 5 | 469.3 | 0.662 |
| I-79 | 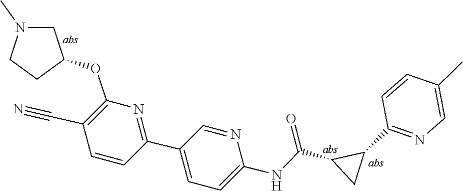 | LCMS 5 | 455.3 | 0.604 |
| I-80 | 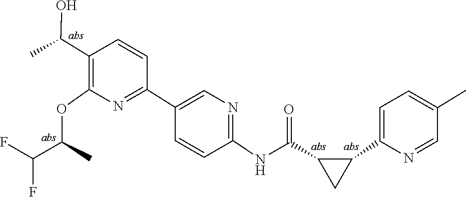 [SMILES: Cc1ccc([C@@H]2C[C@@H]2C(=O)Nc2ccc(-c3ccc([C@H](C)O)c(O[C@@H](C)C(F)F)n3)cn2)nc1] | LCMS 5 | 469.3 | 0.646 |
| I-81 | 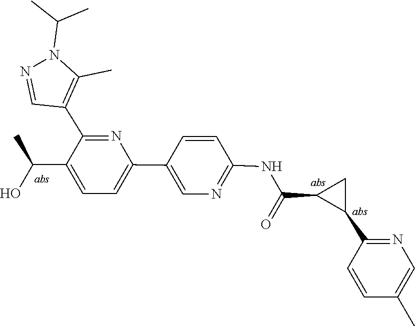 [SMILES: Cc1ccc([C@@H]2C[C@@H]2C(=O)Nc2ccc(-c3ccc([C@H](C)O)c(-c4cnn(C(C)C)c4C)n3)cn2)nc1] | LCMS 2 | 497.25 | 1.21 |
| I-82 | 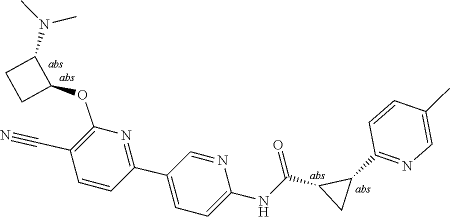 [SMILES: Cc1ccc([C@@H]2C[C@@H]2C(=O)Nc2ccc(-c3ccc(C#N)c(O[C@H]4CC[C@@H]4N(C)C)n3)cn2)nc1] | LCMS 2 | 469.2 | 1.62 |
| I-83 | 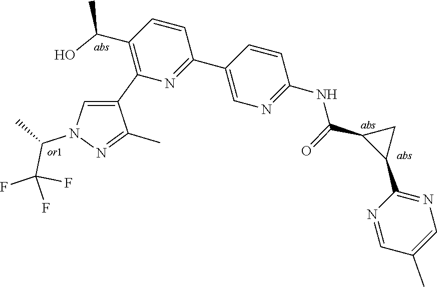 [SMILES: Cc1cnc([C@@H]2C[C@@H]2C(=O)Nc2ccc(-c3ccc([C@H](C)O)c(-c4cn([C@@H](C)C(F)(F)F)nc4C)n3)cn2)nc1] | LCMS 5 | 552.3 | 0.559 |
| I-84 | 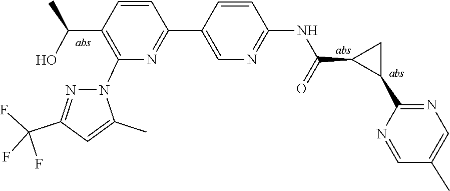 [SMILES: Cc1cnc([C@@H]2C[C@@H]2C(=O)Nc2ccc(-c3ccc([C@H](C)O)c(-n4nc(C(F)(F)F)cc4C)n3)cn2)nc1] | LCMS 5 | 524.3 | 0.576 |
| I-85 | 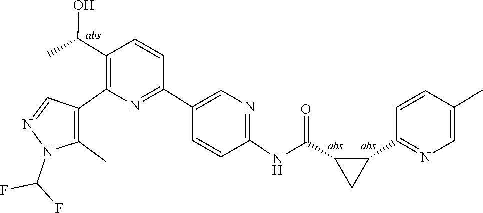 [SMILES: Cc1ccc([C@@H]2C[C@@H]2C(=O)Nc2ccc(-c3ccc([C@H](C)O)c(-c4cnn(C(F)F)c4C)n3)cn2)nc1] | LCMS 4 | 505.1 | 0.414 |
| I-86 | 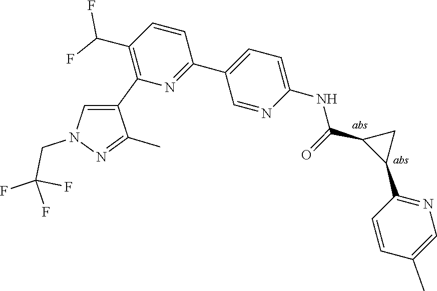 [SMILES: Cc1ccc([C@@H]2C[C@@H]2C(=O)Nc2ccc(-c3ccc(C(F)F)c(-c4cn(CC(F)(F)F)nc4C)n3)cn2)nc1] | LCMS 4 | 543.1 | 0.476 |
| I-87 | 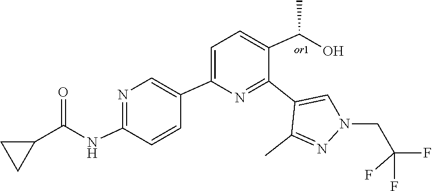 [SMILES: Cc1nn(CC(F)(F)F)cc1-c1nc(-c2ccc(NC(=O)C3CC3)nc2)ccc1[C@H](C)O] | LCMS 2 | 446.15 | 1.11 |
| I-88 | 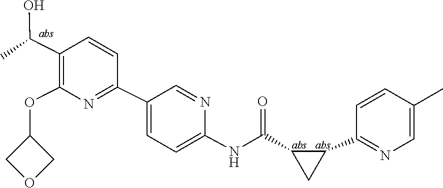 [SMILES: Cc1ccc([C@@H]2C[C@@H]2C(=O)Nc2ccc(-c3ccc([C@H](C)O)c(OC4COC4)n3)cn2)nc1] | LCMS 5 | 447.3 | 0.554 |
| I-89 | 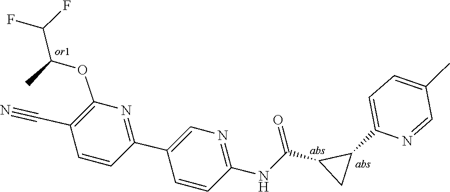 [SMILES: Cc1ccc([C@@H]2C[C@@H]2C(=O)Nc2ccc(-c3ccc(C#N)c(O[C@@H](C)C(F)F)n3)cn2)nc1] | LCMS 5 | 450.2 | 0.662 |
| I-90 | 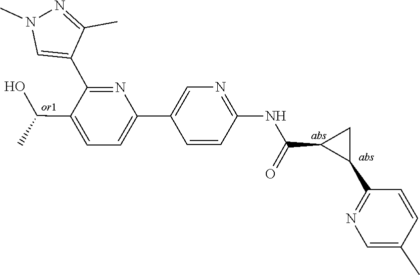 | LCMS 2 | 469.2 | 1.44 |
| I-91 | 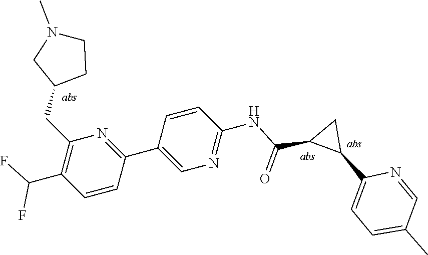 [SMILES: Cc1ccc([C@@H]2C[C@@H]2C(=O)Nc2ccc(-c3ccc(C(F)F)c(C[C@H]4CCN(C)C4)n3)cn2)nc1] | LCMS 3 | 480.2 | 0.84 |
| I-92 | 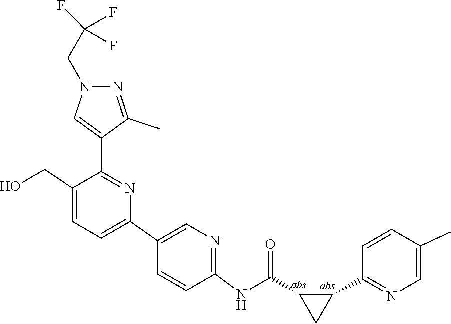 [SMILES: Cc1ccc([C@@H]2C[C@@H]2C(=O)Nc2ccc(-c3ccc(CO)c(-c4cn(CC(F)(F)F)nc4C)n3)cn2)nc1] | LCMS 2 | 523.15 | 1.26 |
| I-93 | 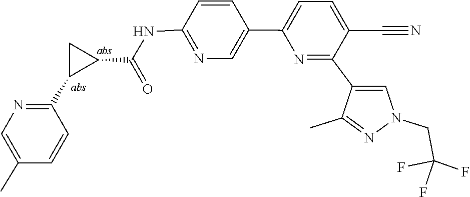 [SMILES: Cc1ccc([C@@H]2C[C@@H]2C(=O)Nc2ccc(-c3ccc(C#N)c(-c4cn(CC(F)(F)F)nc4C)n3)cn2)nc1] | LCMS 1 | 518.1 | 1 |
| I-94 | 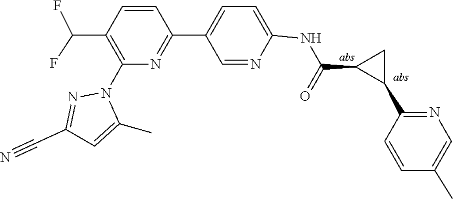 [SMILES: Cc1ccc([C@@H]2C[C@@H]2C(=O)Nc2ccc(-c3ccc(C(F)F)c(-n4nc(C#N)cc4C)n3)cn2)nc1] | LCMS 4 | 486.1 | 0.474 |
| I-95 | 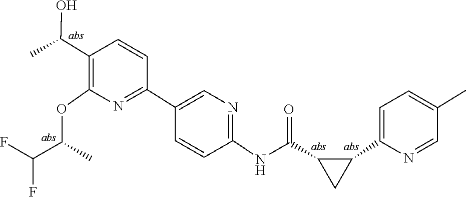 [SMILES: Cc1ccc([C@@H]2C[C@@H]2C(=O)Nc2ccc(-c3ccc([C@H](C)O)c(O[C@H](C)C(F)F)n3)cn2)nc1] | LCMS 4 | 469.2 | 0.456 |
| I-96 | 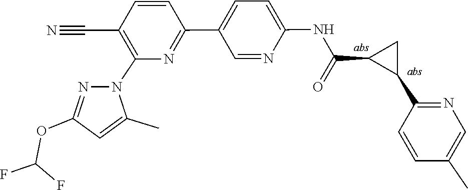 [SMILES: Cc1ccc([C@@H]2C[C@@H]2C(=O)Nc2ccc(-c3ccc(C#N)c(-n4nc(OC(F)F)cc4C)n3)cn2)nc1] | LCMS 4 | 502.3 | 0.478 |
| I-97 | 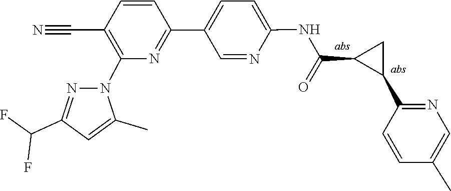 [SMILES: Cc1ccc([C@@H]2C[C@@H]2C(=O)Nc2ccc(-c3ccc(C#N)c(-n4nc(C(F)F)cc4C)n3)cn2)nc1] | LCMS 4 | 486.2 | 0.463 |
| I-98 | 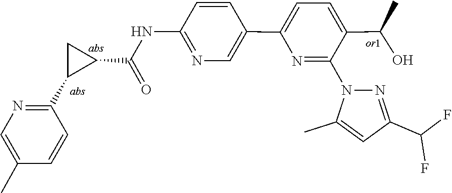 [SMILES: Cc1ccc([C@@H]2C[C@@H]2C(=O)Nc2ccc(-c3ccc([C@@H](C)O)c(-n4nc(C(F)F)cc4C)n3)cn2)nc1] | LCMS 2 | 505.2 | 1.19 |
| I-99 | 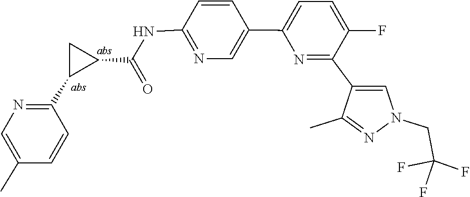 [SMILES: Cc1ccc([C@@H]2C[C@@H]2C(=O)Nc2ccc(-c3ccc(F)c(-c4cn(CC(F)(F)F)nc4C)n3)cn2)nc1] | LCMS 2 | 511.1 | 1.05 |
| I-100 | 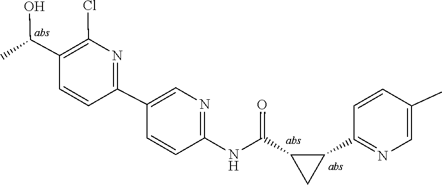 [SMILES: Cc1ccc([C@@H]2C[C@@H]2C(=O)Nc2ccc(-c3ccc([C@H](C)O)c(Cl)n3)cn2)nc1] | LCMS 4 | 409.2 | 0.408 |
| I-101 | 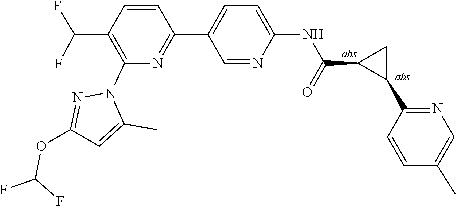 [SMILES: Cc1ccc([C@@H]2C[C@@H]2C(=O)Nc2ccc(-c3ccc(C(F)F)c(-n4nc(OC(F)F)cc4C)n3)cn2)nc1] | LCMS 4 | 527.1 | 0.508 |
| I-102 | 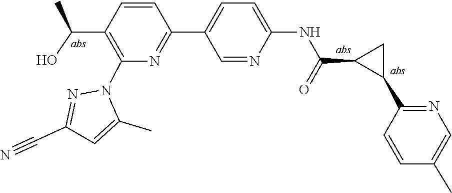 [SMILES: Cc1ccc([C@@H]2C[C@@H]2C(=O)Nc2ccc(-c3ccc([C@H](C)O)c(-n4nc(C#N)cc4C)n3)cn2)nc1] | LCMS 5 | 480.3 | 0.586 |
| I-103 | 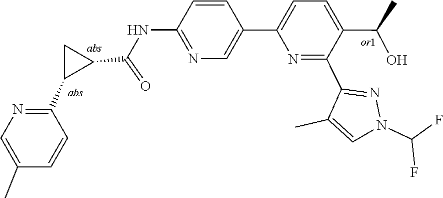 [SMILES: Cc1ccc([C@@H]2C[C@@H]2C(=O)Nc2ccc(-c3ccc([C@@H](C)O)c(-c4nn(C(F)F)cc4C)n3)cn2)nc1] | LCMS 2 | 505.2 | 1.43 |
| I-104 | 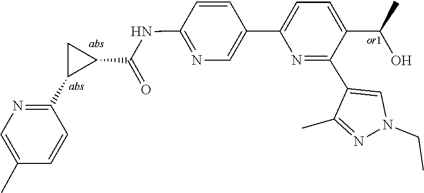 [SMILES: CCn1cc(-c2nc(-c3ccc(NC(=O)[C@H]4C[C@H]4c4ccc(C)cn4)nc3)ccc2[C@@H](C)O)c(C)n1] | LCMS 2 | 483.25 | 1.01 |
| I-105 | 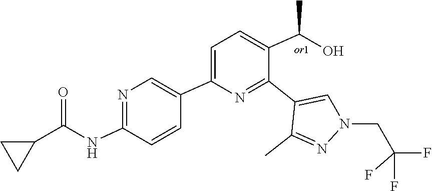 [SMILES: Cc1nn(CC(F)(F)F)cc1-c1nc(-c2ccc(NC(=O)C3CC3)nc2)ccc1[C@@H](C)O] | LCMS 2 | 446.15 | 1.11 |
| I-106 | 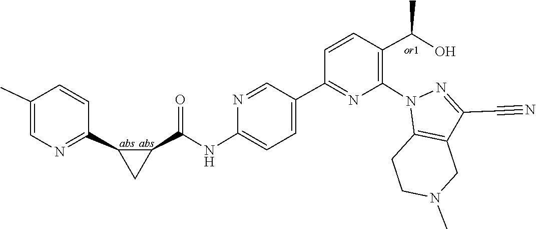 [SMILES: Cc1ccc([C@@H]2C[C@@H]2C(=O)Nc2ccc(-c3ccc([C@@H](C)O)c(-n4nc(C#N)c5c4CCN(C)C5)n3)cn2)nc1] | LCMS 2 | 535.25 | 1.25 |
| I-107 | 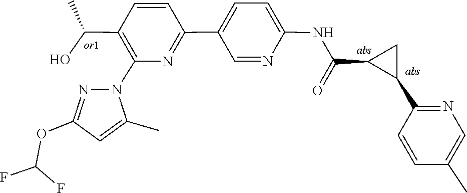 [SMILES: Cc1ccc([C@@H]2C[C@@H]2C(=O)Nc2ccc(-c3ccc([C@@H](C)O)c(-n4nc(OC(F)F)cc4C)n3)cn2)nc1] | LCMS 4 | 521.3 | 0.436 |
| I-108 | 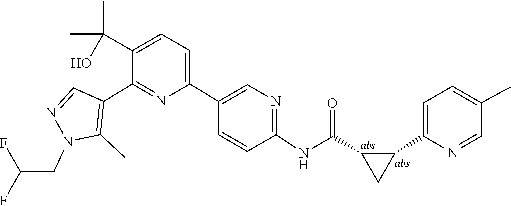 [SMILES: Cc1ccc([C@@H]2C[C@@H]2C(=O)Nc2ccc(-c3ccc(C(C)(C)O)c(-c4cnn(CC(F)F)c4C)n3)cn2)nc1] | LCMS 5 | 533.3 | 0.551 |
| I-109 | 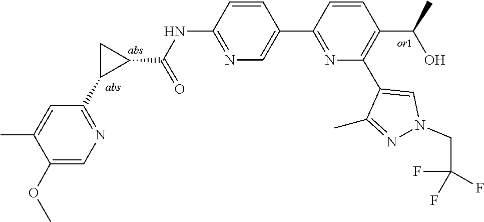 [SMILES: COc1cnc([C@@H]2C[C@@H]2C(=O)Nc2ccc(-c3ccc([C@@H](C)O)c(-c4cn(CC(F)(F)F)nc4C)n3)cn2)cc1C] | LCMS 2 | 567.25 | 1.34 |
| I-110 | 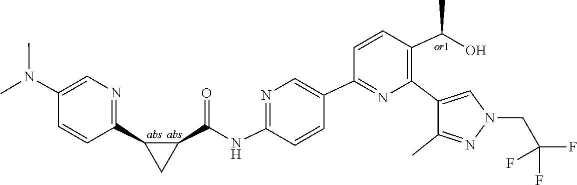 [SMILES: Cc1nn(CC(F)(F)F)cc1-c1nc(-c2ccc(NC(=O)[C@H]3C[C@H]3c3ccc(N(C)C)cn3)nc2)ccc1[C@@H](C)O] | LCMS 2 | 566.25 | 1.27 |
| I-111 | 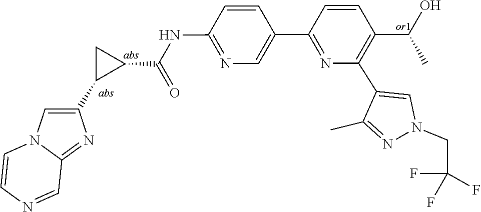 [SMILES: Cc1nn(CC(F)(F)F)cc1-c1nc(-c2ccc(NC(=O)[C@H]3C[C@H]3c3cn4ccncc4n3)nc2)ccc1[C@@H](C)O] | LCMS 2 | 563.25 | 1.38 |
| I-112 | 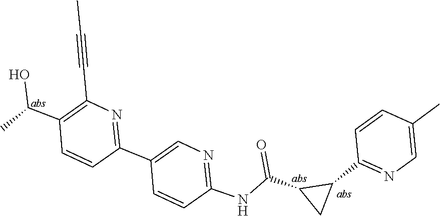 [SMILES: CC#Cc1nc(-c2ccc(NC(=O)[C@H]3C[C@H]3c3ccc(C)cn3)nc2)ccc1[C@H](C)O] | LCMS 5 | 413.3 | 0.544 |
| I-113 | 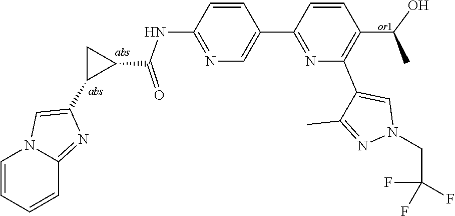 [SMILES: Cc1nn(CC(F)(F)F)cc1-c1nc(-c2ccc(NC(=O)[C@H]3C[C@H]3c3cn4ccccc4n3)nc2)ccc1[C@H](C)O] | LCMS 2 | 562.2 | 1.59 |
| I-114 | 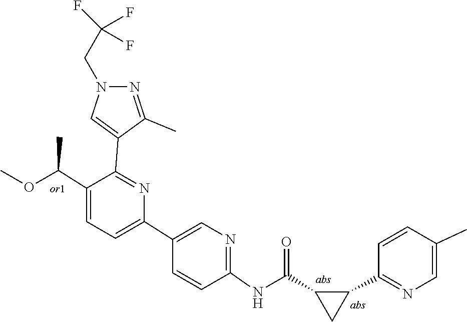 | LCMS 1 | 551.25 | 1.7 |
| I-115 | 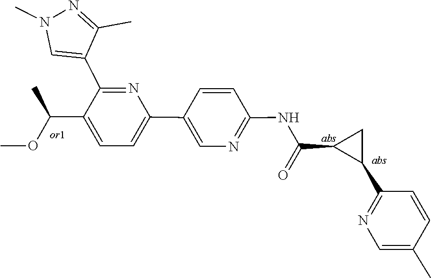 | LCMS 2 | 483.25 | 1.23 |
| I-116 | 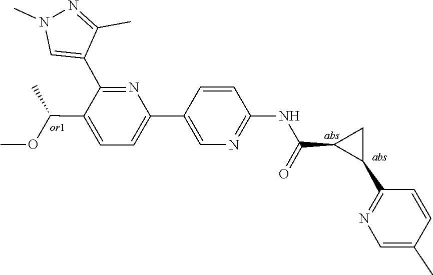 | LCMS 2 | 483.25 | 1.24 |
| I-117 | 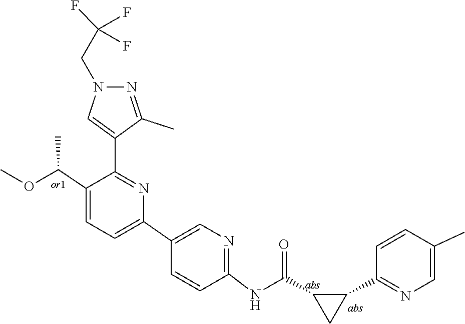 | LCMS 2 | 551.25 | 1.22 |
| I-118 | 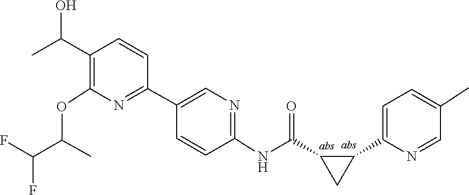 [SMILES: Cc1ccc([C@@H]2C[C@@H]2C(=O)Nc2ccc(-c3ccc(C(C)O)c(OC(C)C(F)F)n3)cn2)nc1] | LCMS 5 | 469.3 | 0.615 |
| I-119 | 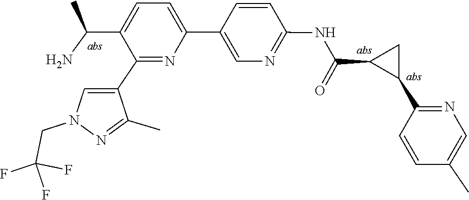 [SMILES: Cc1ccc([C@@H]2C[C@@H]2C(=O)Nc2ccc(-c3ccc([C@H](C)N)c(-c4cn(CC(F)(F)F)nc4C)n3)cn2)nc1] | LCMS 5 | 536.4 | 0.56 |
| I-120 | 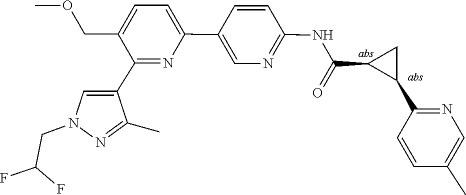 [SMILES: COCc1ccc(-c2ccc(NC(=O)[C@H]3C[C@H]3c3ccc(C)cn3)nc2)nc1-c1cn(CC(F)F)nc1C] | LCMS 5 | 519.4 | 0.634 |
| I-121 | 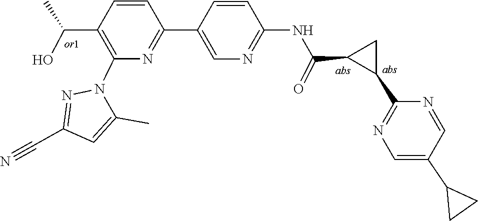 [SMILES: Cc1cc(C#N)nn1-c1nc(-c2ccc(NC(=O)[C@H]3C[C@H]3c3ncc(C4CC4)cn3)nc2)ccc1[C@@H](C)O] | LCMS 5 | 507.3 | 0.552 |
| I-122 | 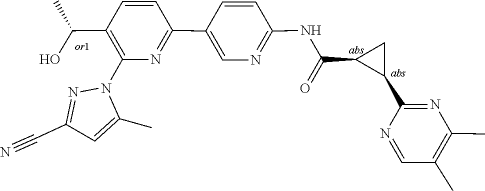 [SMILES: Cc1cnc([C@@H]2C[C@@H]2C(=O)Nc2ccc(-c3ccc([C@@H](C)O)c(-n4nc(C#N)cc4C)n3)cn2)nc1C] | LCMS 5 | 495.4 | 0.566 |
| I-123 | 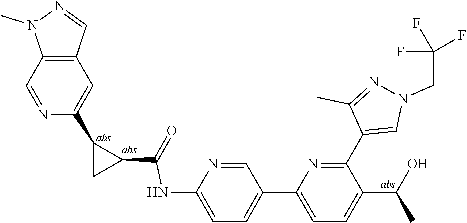 [SMILES: Cc1nn(CC(F)(F)F)cc1-c1nc(-c2ccc(NC(=O)[C@H]3C[C@H]3c3cc4cnn(C)c4cn3)nc2)ccc1[C@H](C)O] | LCMS 2 | 577.15 | 1.33 |
| I-124 | 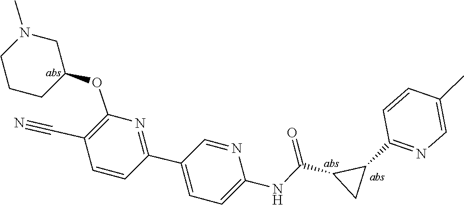 [SMILES: Cc1ccc([C@@H]2C[C@@H]2C(=O)Nc2ccc(-c3ccc(C#N)c(O[C@H]4CCCN(C)C4)n3)cn2)nc1] | LCMS 2 | 469.15 | 1.5 |
| I-125 | 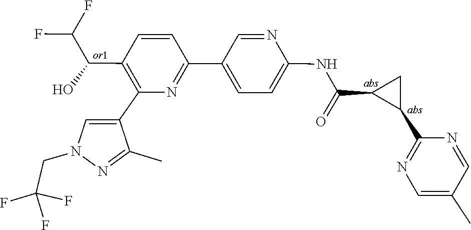 [SMILES: Cc1cnc([C@@H]2C[C@@H]2C(=O)Nc2ccc(-c3ccc([C@H](O)C(F)F)c(-c4cn(CC(F)(F)F)nc4C)n3)cn2)nc1] | LCMS 5 | 574.4 | 0.551 |
| I-126 | 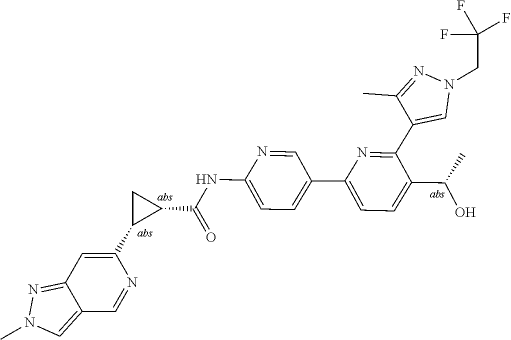 [SMILES: Cc1nn(CC(F)(F)F)cc1-c1nc(-c2ccc(NC(=O)[C@H]3C[C@H]3c3cc4nn(C)cc4cn3)nc2)ccc1[C@H](C)O] | LCMS 2 | 577.2 | 1.24 |
| I-127 | 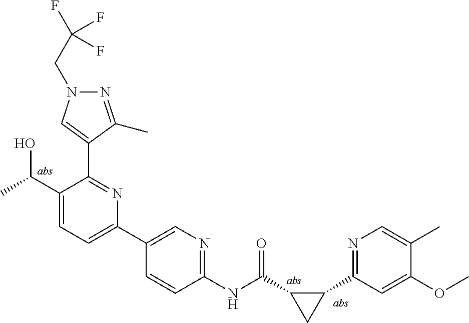 [SMILES: COc1cc([C@@H]2C[C@@H]2C(=O)Nc2ccc(-c3ccc([C@H](C)O)c(-c4cn(CC(F)(F)F)nc4C)n3)cn2)ncc1C] | LCMS 2 | 567.2 | 1.39 |
| I-128 | 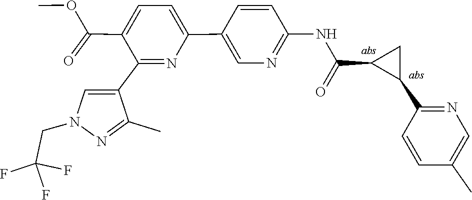 [SMILES: COC(=O)c1ccc(-c2ccc(NC(=O)[C@H]3C[C@H]3c3ccc(C)cn3)nc2)nc1-c1cn(CC(F)(F)F)nc1C] | LCMS 2 | 551.2 | 1.58 |
| I-129 | 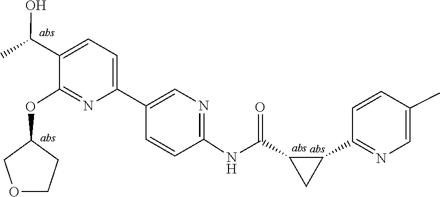 [SMILES: Cc1ccc([C@@H]2C[C@@H]2C(=O)Nc2ccc(-c3ccc([C@H](C)O)c(O[C@H]4CCOC4)n3)cn2)nc1] | LCMS 5 | 461.3 | 0.569 |
| I-130 | 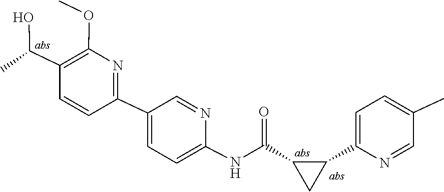 [SMILES: COc1nc(-c2ccc(NC(=O)[C@H]3C[C@H]3c3ccc(C)cn3)nc2)ccc1[C@H](C)O] | LCMS 4 | 405.2 | 0.404 |
| I-131 | 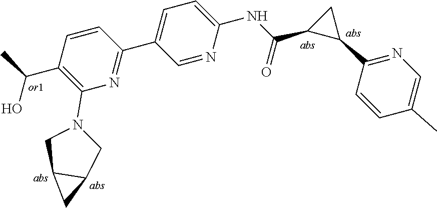 [SMILES: Cc1ccc([C@@H]2C[C@@H]2C(=O)Nc2ccc(-c3ccc([C@H](C)O)c(N4C[C@H]5C[C@H]5C4)n3)cn2)nc1] | LCMS 5 | 456.3 | 0.627 |
| I-132 | 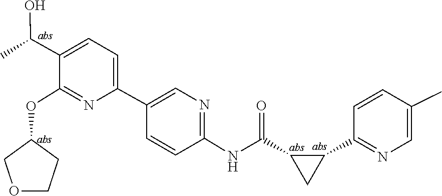 [SMILES: Cc1ccc([C@@H]2C[C@@H]2C(=O)Nc2ccc(-c3ccc([C@H](C)O)c(O[C@@H]4CCOC4)n3)cn2)nc1] | LCMS 5 | 461.3 | 0.575 |
| I-133 | 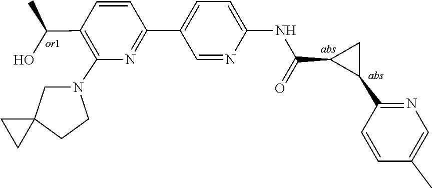 [SMILES: Cc1ccc([C@@H]2C[C@@H]2C(=O)Nc2ccc(-c3ccc([C@H](C)O)c(N4CCC5(CC5)C4)n3)cn2)nc1] | LCMS 5 | 470.3 | 0.651 |
| I-134 | 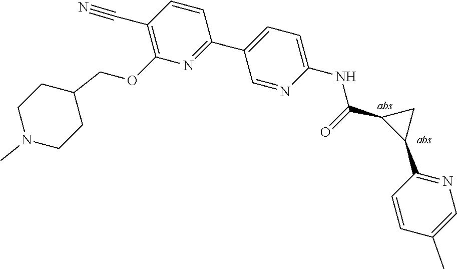 [SMILES: Cc1ccc([C@@H]2C[C@@H]2C(=O)Nc2ccc(-c3ccc(C#N)c(OCC4CCN(C)CC4)n3)cn2)nc1] | LCMS 5 | 483.4 | 0.788 |
| I-135 | 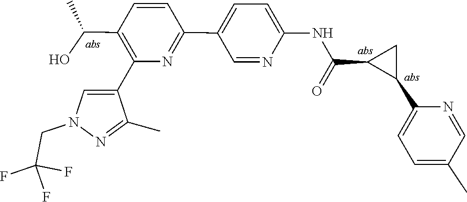 [SMILES: Cc1ccc([C@@H]2C[C@@H]2C(=O)Nc2ccc(-c3ccc([C@@H](C)O)c(-c4cn(CC(F)(F)F)nc4C)n3)cn2)nc1] | LCMS 4 | 537.3 | 0.422 |
| I-136 | 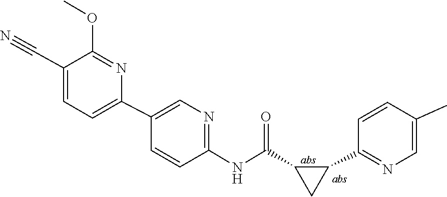 [SMILES: COc1nc(-c2ccc(NC(=O)[C@H]3C[C@H]3c3ccc(C)cn3)nc2)ccc1C#N] | LCMS 2 | 386.1 | 1.49 |
| I-137 | 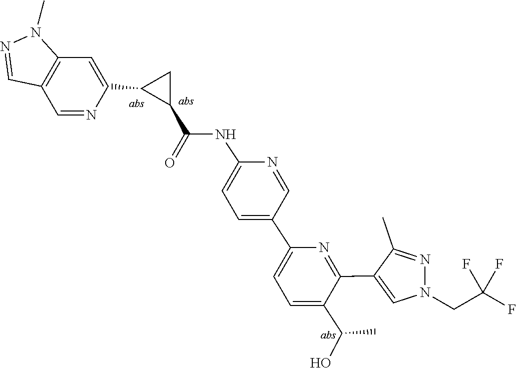 [SMILES: Cc1nn(CC(F)(F)F)cc1-c1nc(-c2ccc(NC(=O)[C@@H]3C[C@H]3c3cc4c(cn3)cnn4C)nc2)ccc1[C@H](C)O] | LCMS 2 | 577.2 | 1.08 |
| I-138 | 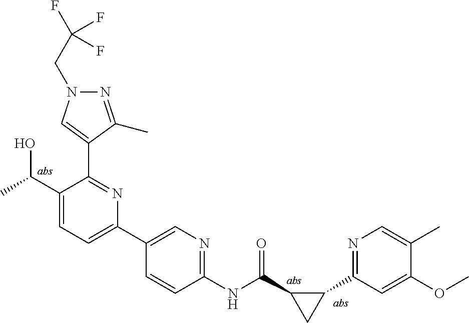 [SMILES: COc1cc([C@@H]2C[C@H]2C(=O)Nc2ccc(-c3ccc([C@H](C)O)c(-c4cn(CC(F)(F)F)nc4C)n3)cn2)ncc1C] | LCMS 2 | 567.2 | 1.54 |
| I-139 | 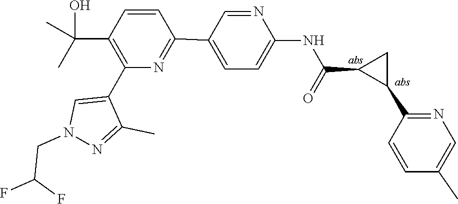 [SMILES: Cc1ccc([C@@H]2C[C@@H]2C(=O)Nc2ccc(-c3ccc(C(C)(C)O)c(-c4cn(CC(F)F)nc4C)n3)cn2)nc1] | LCMS 5 | 533.3 | 0.545 |
| I-140 | 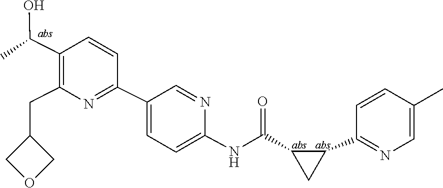 [SMILES: Cc1ccc([C@@H]2C[C@@H]2C(=O)Nc2ccc(-c3ccc([C@H](C)O)c(CC4COC4)n3)cn2)nc1] | LCMS 5 | 445.3 | 0.513 |
| I-141 | 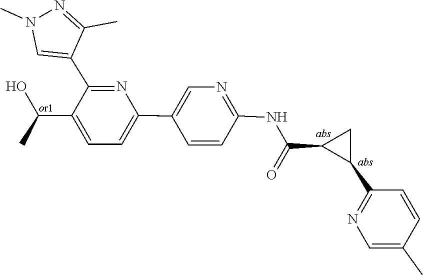 | LCMS 2 | 469.15 | 1.43 |
| I-142 | 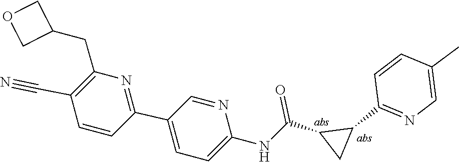 [SMILES: Cc1ccc([C@@H]2C[C@@H]2C(=O)Nc2ccc(-c3ccc(C#N)c(CC4COC4)n3)cn2)nc1] | LCMS 5 | 426.2 | 0.562 |
| I-143 | 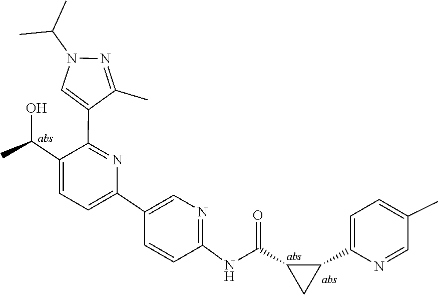 [SMILES: Cc1ccc([C@@H]2C[C@@H]2C(=O)Nc2ccc(-c3ccc([C@@H](C)O)c(-c4cn(C(C)C)nc4C)n3)cn2)nc1] | LCMS 2 | 497.25 | 1.19 |
| I-144 | 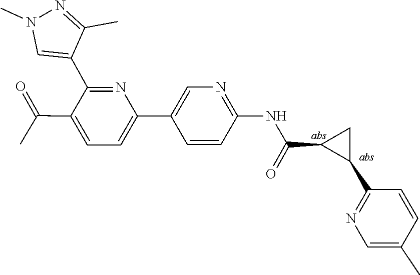 | LCMS 2 | 467.25 | 0.89 |
| I-145 | 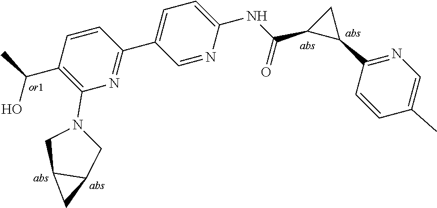 [SMILES: Cc1ccc([C@@H]2C[C@@H]2C(=O)Nc2ccc(-c3ccc([C@H](C)O)c(N4C[C@H]5C[C@H]5C4)n3)cn2)nc1] | LCMS 5 | 456.3 | 0.63 |
| I-146 | 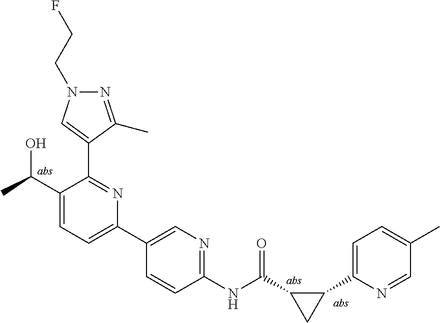 [SMILES: Cc1ccc([C@@H]2C[C@@H]2C(=O)Nc2ccc(-c3ccc([C@@H](C)O)c(-c4cn(CCF)nc4C)n3)cn2)nc1] | LCMS 2 | 501.15 | 1.02 |
| I-147 | 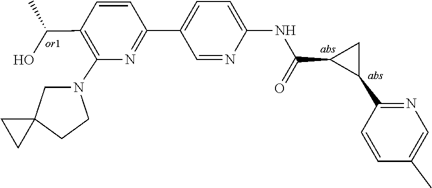 [SMILES: Cc1ccc([C@@H]2C[C@@H]2C(=O)Nc2ccc(-c3ccc([C@@H](C)O)c(N4CCC5(CC5)C4)n3)cn2)nc1] | LCMS 5 | 470.3 | 0.657 |
| I-148 | 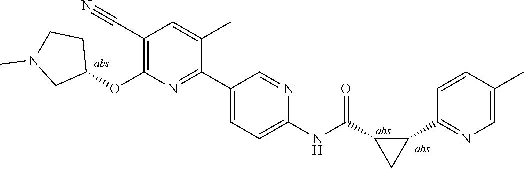 | LCMS 2 | 469.2 | 1.48 |
| I-149 | 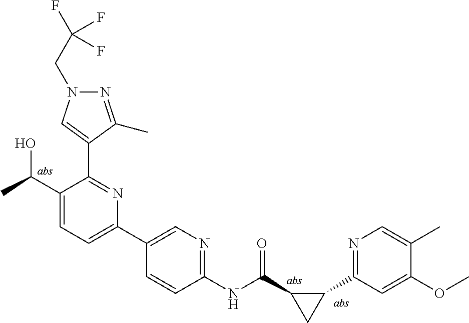 [SMILES: COc1cc([C@@H]2C[C@H]2C(=O)Nc2ccc(-c3ccc([C@@H](C)O)c(-c4cn(CC(F)(F)F)nc4C)n3)cn2)ncc1C] | LCMS 1 | 567.2 | 0.96 |
| I-150 | 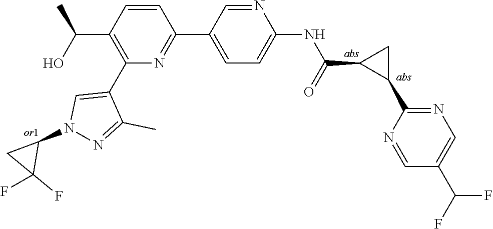 [SMILES: Cc1nn([C@@H]2CC2(F)F)cc1-c1nc(-c2ccc(NC(=O)[C@H]3C[C@H]3c3ncc(C(F)F)cn3)nc2)ccc1[C@H](C)O] | LCMS-5 | 568.4 | 0.526 |
| I-151 | 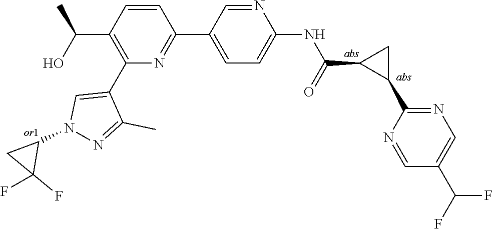 | LCMS-5 | 568.2 | 0.522 |
| I-152 | 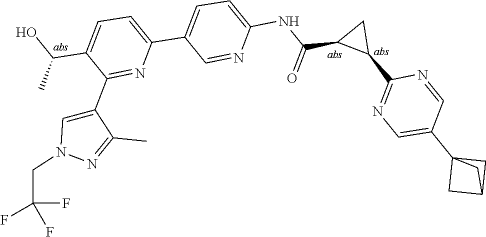 [SMILES: Cc1nn(CC(F)(F)F)cc1-c1nc(-c2ccc(NC(=O)[C@H]3C[C@H]3c3ncc(C45CC(C4)C5)cn3)nc2)ccc1[C@H](C)O] | LCMS 4 | 590.3 | 0.497 |
| I-153 | 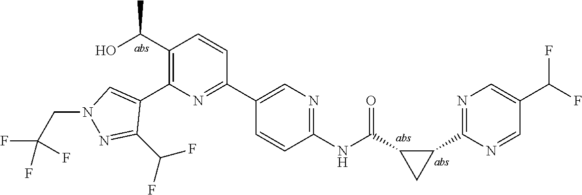 [SMILES: C[C@H](O)c1ccc(-c2ccc(NC(=O)[C@H]3C[C@H]3c3ncc(C(F)F)cn3)nc2)nc1-c1cn(CC(F)(F)F)nc1C(F)F] | LCMS 5 | 610.2 | 0.59 |
| I-154 | 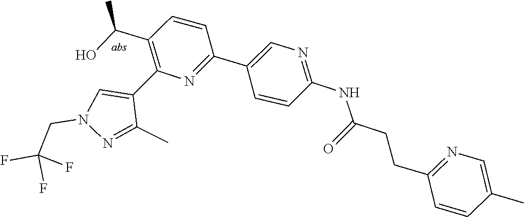 [SMILES: Cc1ccc(CCC(=O)Nc2ccc(-c3ccc([C@H](C)O)c(-c4cn(CC(F)(F)F)nc4C)n3)cn2)nc1] | LCMS 4 | 525.3 | 0.408 |
| I-155 | 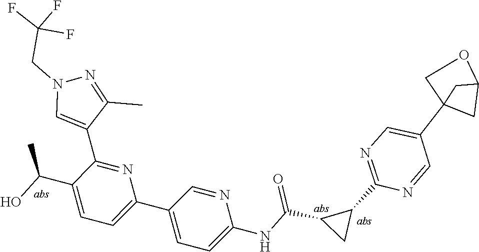 [SMILES: Cc1nn(CC(F)(F)F)cc1-c1nc(-c2ccc(NC(=O)[C@H]3C[C@H]3c3ncc(C45COC(C4)C5)cn3)nc2)ccc1[C@H](C)O] | LCMS 5 | 606.2 | 0.495 |
| I-156 | 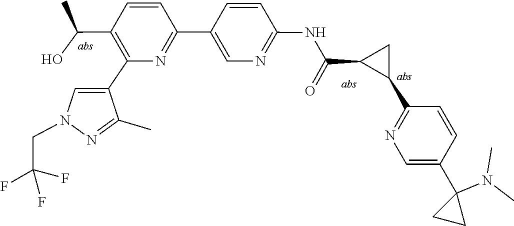 [SMILES: Cc1nn(CC(F)(F)F)cc1-c1nc(-c2ccc(NC(=O)[C@H]3C[C@H]3c3ccc(C4(N(C)C)CC4)cn3)nc2)ccc1[C@H](C)O] | LCMS 5 | 606.5 | 0.55 |
| I-157 | 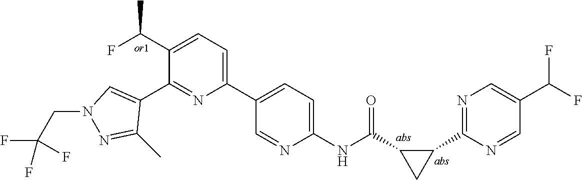 [SMILES: Cc1nn(CC(F)(F)F)cc1-c1nc(-c2ccc(NC(=O)[C@H]3C[C@H]3c3ncc(C(F)F)cn3)nc2)ccc1[C@H](C)F] | LCMS 4 | 576.3 | 0.529 |
| I-158 | 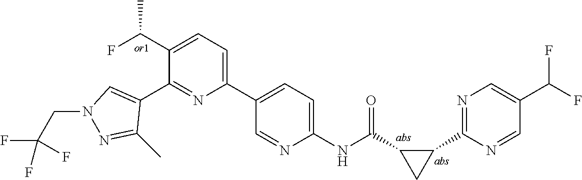 [SMILES: Cc1nn(CC(F)(F)F)cc1-c1nc(-c2ccc(NC(=O)[C@H]3C[C@H]3c3ncc(C(F)F)cn3)nc2)ccc1[C@@H](C)F] | LCMS 4 | 576.3 | 0.53 |
| I-159 | 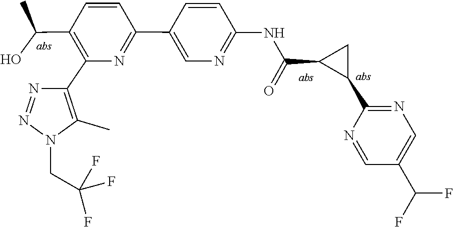 [SMILES: Cc1c(-c2nc(-c3ccc(NC(=O)[C@H]4C[C@H]4c4ncc(C(F)F)cn4)nc3)ccc2[C@H](C)O)nnn1CC(F)(F)F] | LCMS 5 | 575.3 | 0.567 |
| I-160 | 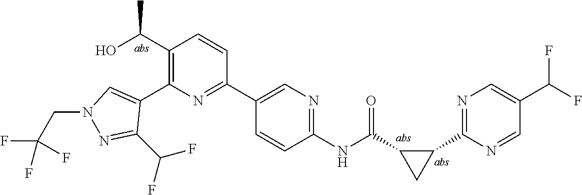 | LCMS 5 | 575.2 | 0.607 |
| I-161 | 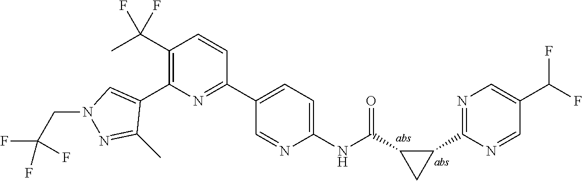 [SMILES: Cc1nn(CC(F)(F)F)cc1-c1nc(-c2ccc(NC(=O)[C@H]3C[C@H]3c3ncc(C(F)F)cn3)nc2)ccc1C(C)(F)F] | LCMS 5 | 594.2 | 0.656 |
| I-162 | 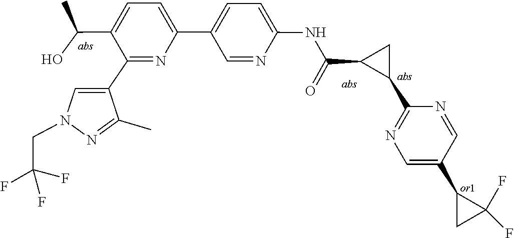 [SMILES: Cc1nn(CC(F)(F)F)cc1-c1nc(-c2ccc(NC(=O)[C@H]3C[C@H]3c3ncc([C@@H]4CC4(F)F)cn3)nc2)ccc1[C@H](C)O] | LCMS 4 | 600.3 | 0.452 |
| I-163 | 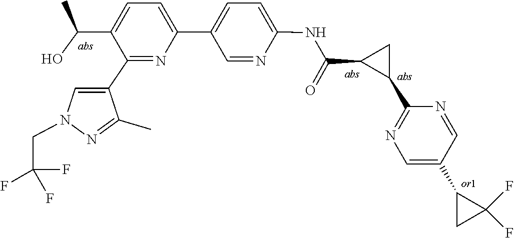 [SMILES: Cc1nn(CC(F)(F)F)cc1-c1nc(-c2ccc(NC(=O)[C@H]3C[C@H]3c3ncc([C@H]4CC4(F)F)cn3)nc2)ccc1[C@H](C)O] | LCMS 4 | 600.3 | 0.455 |
| I-164 | 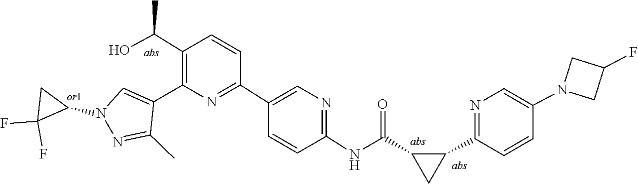 [SMILES: Cc1nn([C@H]2CC2(F)F)cc1-c1nc(-c2ccc(NC(=O)[C@H]3C[C@H]3c3ccc(N4CC(F)C4)cn3)nc2)ccc1[C@H](C)O] | LCMS 4 | 590.3 | 0.434 |
| I-165 | 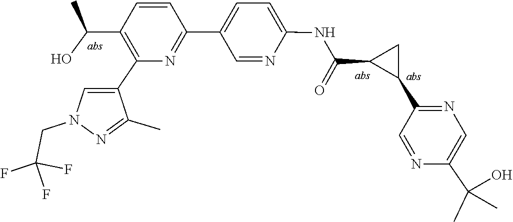 [SMILES: Cc1nn(CC(F)(F)F)cc1-c1nc(-c2ccc(NC(=O)[C@H]3C[C@H]3c3cnc(C(C)(C)O)cn3)nc2)ccc1[C@H](C)O] | LCMS 4 | 582.3 | 0.433 |
| I-166 | 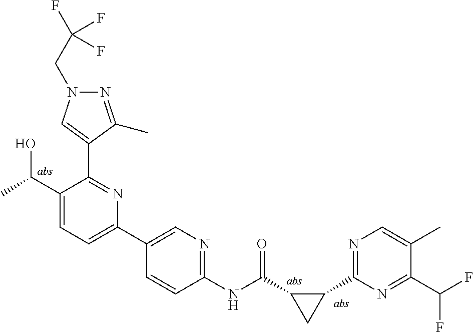 [SMILES: Cc1cnc([C@@H]2C[C@@H]2C(=O)Nc2ccc(-c3ccc([C@H](C)O)c(-c4cn(CC(F)(F)F)nc4C)n3)cn2)nc1C(F)F] | LCMS 2 | 588.25 | 1.26 |
| I-167 | 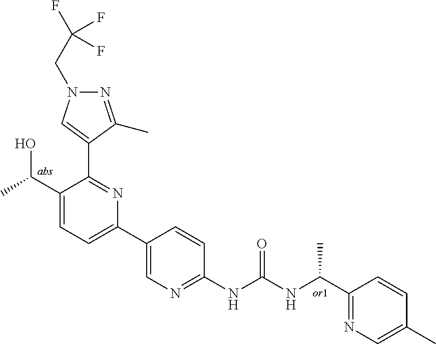 [SMILES: Cc1ccc([C@@H](C)NC(=O)Nc2ccc(-c3ccc([C@H](C)O)c(-c4cn(CC(F)(F)F)nc4C)n3)cn2)nc1] | LCMS 6 | 540.2 | 0.96 |
| I-168 | 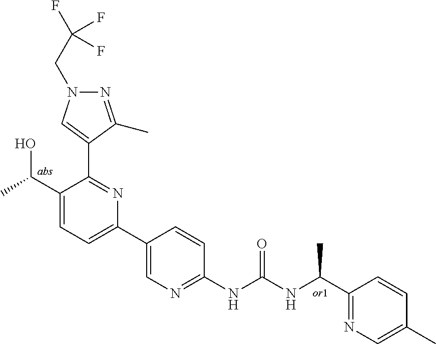 [SMILES: Cc1ccc([C@H](C)NC(=O)Nc2ccc(-c3ccc([C@H](C)O)c(-c4cn(CC(F)(F)F)nc4C)n3)cn2)nc1] | LCMS 2 | 540.25 | 1.29 |
| I-169 | 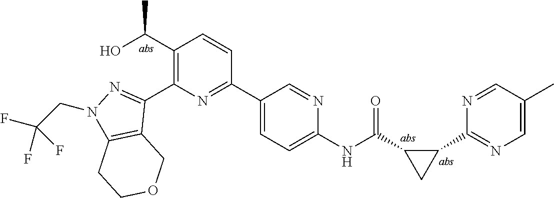 [SMILES: Cc1cnc([C@@H]2C[C@@H]2C(=O)Nc2ccc(-c3ccc([C@H](C)O)c(-c4nn(CC(F)(F)F)c5c4COCC5)n3)cn2)nc1] | LCMS 2 | 580.2 | 1.26 |
| I-170 | 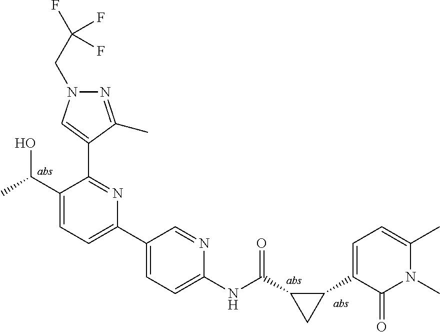 [SMILES: Cc1nn(CC(F)(F)F)cc1-c1nc(-c2ccc(NC(=O)[C@H]3C[C@H]3c3ccc(C)n(C)c3=O)nc2)ccc1[C@H](C)O] | LCMS 1 | 567.15 | 1.26 |
| I-171 | 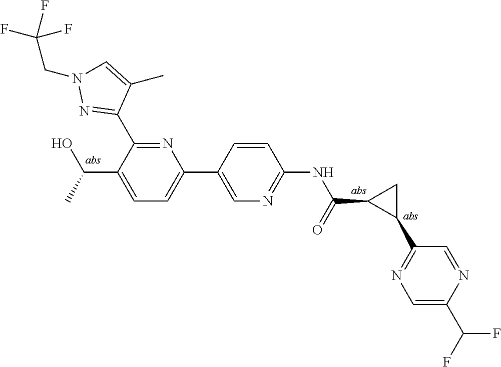 [SMILES: Cc1cn(CC(F)(F)F)nc1-c1nc(-c2ccc(NC(=O)[C@H]3C[C@H]3c3cnc(C(F)F)cn3)nc2)ccc1[C@H](C)O] | LCMS 2 | 574.15 | 1.17 |
| I-172 | 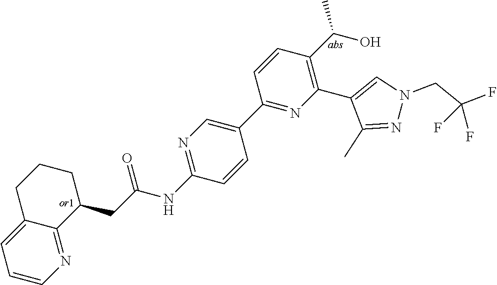 [SMILES: Cc1nn(CC(F)(F)F)cc1-c1nc(-c2ccc(NC(=O)C[C@@H]3CCCc4cccnc43)nc2)ccc1[C@H](C)O] | LCMS 7 | 550.23 | 4.45 |
| I-173 | 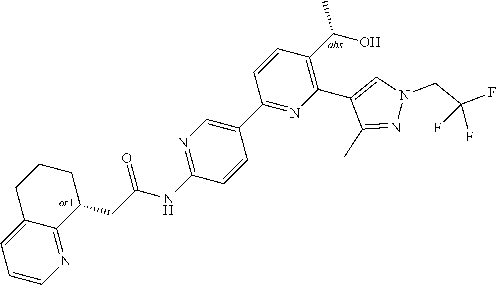 [SMILES: Cc1nn(CC(F)(F)F)cc1-c1nc(-c2ccc(NC(=O)C[C@H]3CCCc4cccnc43)nc2)ccc1[C@H](C)O] | LCMS 7 | 550.23 | 4.45 |
| I-174 | 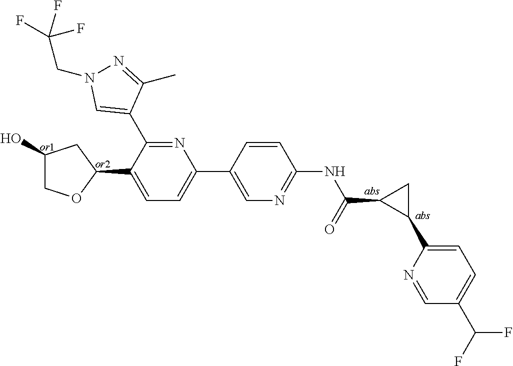 [SMILES: Cc1nn(CC(F)(F)F)cc1-c1nc(-c2ccc(NC(=O)[C@H]3C[C@H]3c3ccc(C(F)F)cn3)nc2)ccc1[C@@H]1C[C@H](O)CO1] | LCMS 2 | 615.25 | 1.21 |
| I-175 | 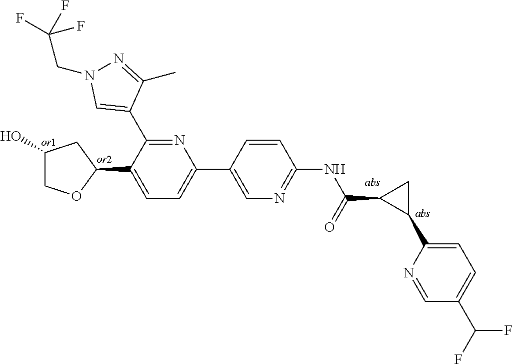 [SMILES: Cc1nn(CC(F)(F)F)cc1-c1nc(-c2ccc(NC(=O)[C@H]3C[C@H]3c3ccc(C(F)F)cn3)nc2)ccc1[C@@H]1C[C@@H](O)CO1] | LCMS 2 | 615.25 | 1.21 |
| I-176 | 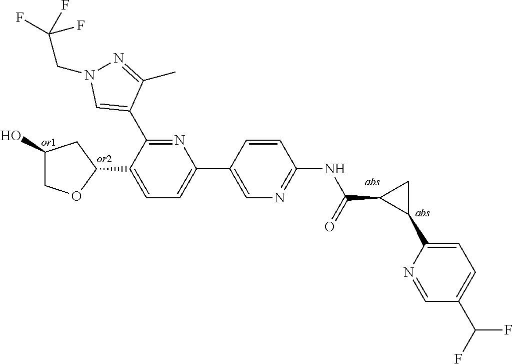 [SMILES: Cc1nn(CC(F)(F)F)cc1-c1nc(-c2ccc(NC(=O)[C@H]3C[C@H]3c3ccc(C(F)F)cn3)nc2)ccc1[C@H]1C[C@H](O)CO1] | LCMS 2 | 615.25 | 1.21 |
| I-177 | 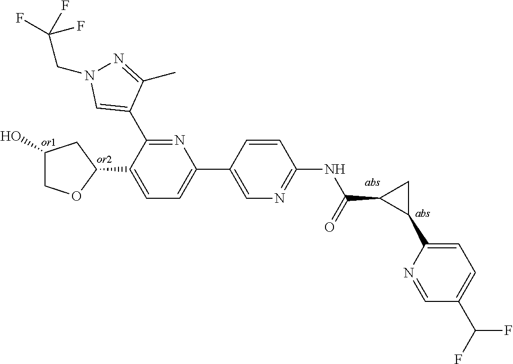 [SMILES: Cc1nn(CC(F)(F)F)cc1-c1nc(-c2ccc(NC(=O)[C@H]3C[C@H]3c3ccc(C(F)F)cn3)nc2)ccc1[C@H]1C[C@@H](O)CO1] | LCMS 2 | 615.25 | 1.25 |
| I-178 | 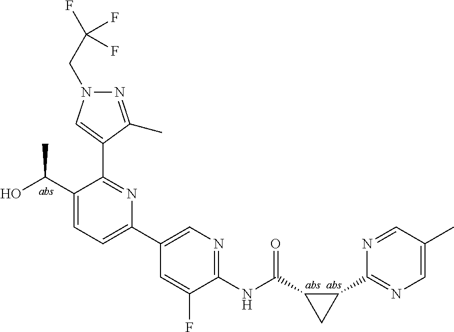 [SMILES: Cc1cnc([C@@H]2C[C@@H]2C(=O)Nc2ncc(-c3ccc([C@H](C)O)c(-c4cn(CC(F)(F)F)nc4C)n3)cc2F)nc1] | LCMS 2 | 556.15 | 0.83 |
| I-179 | 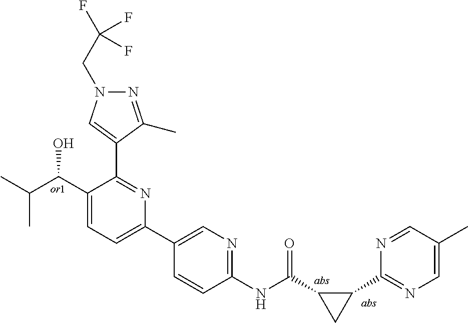 [SMILES: Cc1cnc([C@@H]2C[C@@H]2C(=O)Nc2ccc(-c3ccc([C@@H](O)C(C)C)c(-c4cn(CC(F)(F)F)nc4C)n3)cn2)nc1] | LCMS 2 | 566.25 | 1.32 |
| I-180 | 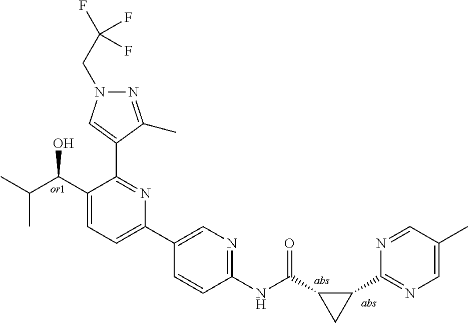 [SMILES: Cc1cnc([C@@H]2C[C@@H]2C(=O)Nc2ccc(-c3ccc([C@H](O)C(C)C)c(-c4cn(CC(F)(F)F)nc4C)n3)cn2)nc1] | LCMS 2 | 566.3 | 1.31 |
| I-181 | 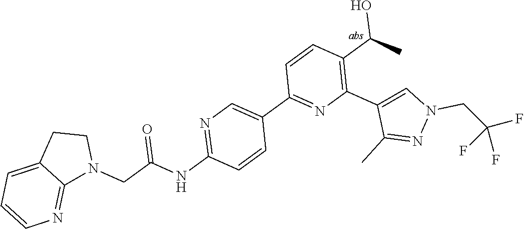 [SMILES: Cc1nn(CC(F)(F)F)cc1-c1nc(-c2ccc(NC(=O)CN3CCc4cccnc43)nc2)ccc1[C@H](C)O] | LCMS-7 | 537.21 | 4.14 |
| I-182 | 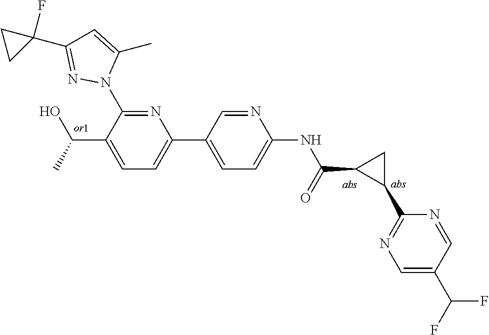 [SMILES: Cc1cc(C2(F)CC2)nn1-c1nc(-c2ccc(NC(=O)[C@H]3C[C@H]3c3ncc(C(F)F)cn3)nc2)ccc1[C@H](C)O] | LCMS-3 | 550.2 | 0.87 |
| I-183 | 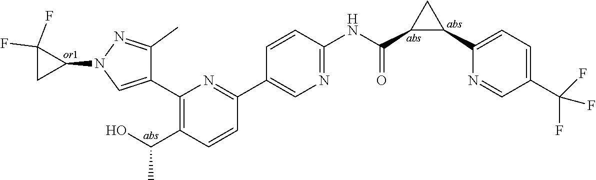 [SMILES: Cc1nn([C@H]2CC2(F)F)cc1-c1nc(-c2ccc(NC(=O)[C@H]3C[C@H]3c3ccc(C(F)(F)F)cn3)nc2)ccc1[C@H](C)O] | LCMS-3 | 585.2 | 0.84 |
| I-184 | 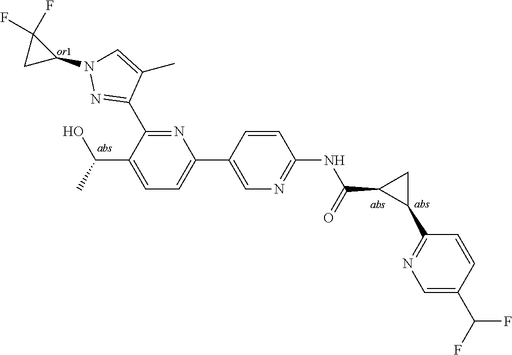 [SMILES: Cc1cn([C@H]2CC2(F)F)nc1-c1nc(-c2ccc(NC(=O)[C@H]3C[C@H]3c3ccc(C(F)F)cn3)nc2)ccc1[C@H](C)O] | LCMS-2 | 567.2 | 1.59 |
| I-185 | 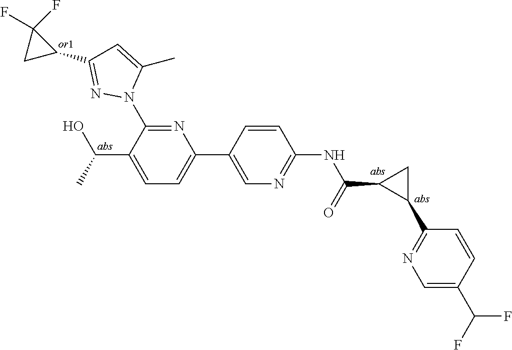 [SMILES: Cc1cc([C@@H]2CC2(F)F)nn1-c1nc(-c2ccc(NC(=O)[C@H]3C[C@H]3c3ccc(C(F)F)cn3)nc2)ccc1[C@H](C)O] | LCMS-2 | 567.25 | 1.57 |
| I-186 | 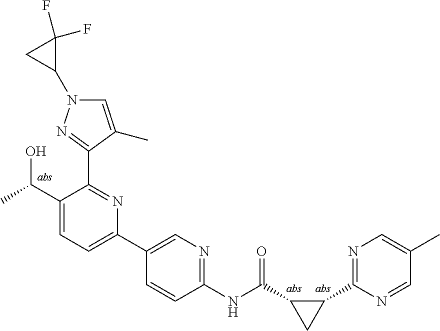 [SMILES: Cc1cnc([C@@H]2C[C@@H]2C(=O)Nc2ccc(-c3ccc([C@H](C)O)c(-c4nn(C5CC5(F)F)cc4C)n3)cn2)nc1] | LCMS-2 | 532.25 | 1.24 |
| I-187 | 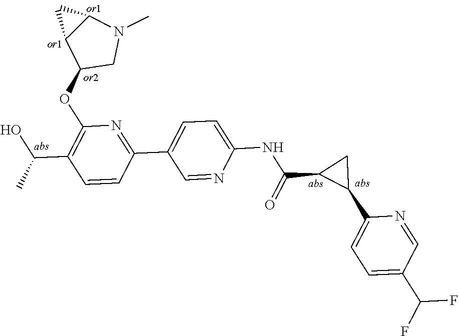 [SMILES: C[C@H](O)c1ccc(-c2ccc(NC(=O)[C@H]3C[C@H]3c3ccc(C(F)F)cn3)nc2)nc1O[C@@H]1CN(C)[C@H]2C[C@@H]12] | LCMS-2 | 522.2 | 1.35 |
| I-188 | 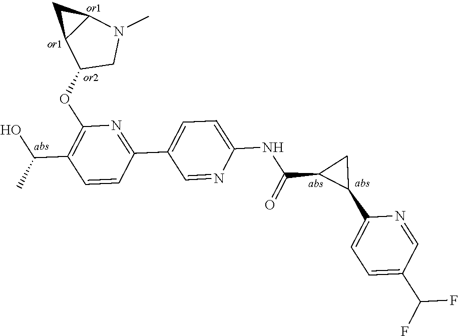 [SMILES: C[C@H](O)c1ccc(-c2ccc(NC(=O)[C@H]3C[C@H]3c3ccc(C(F)F)cn3)nc2)nc1O[C@H]1CN(C)[C@@H]2C[C@H]12] | LCMS-2 | 522.2 | 1.35 |
| I-189 | 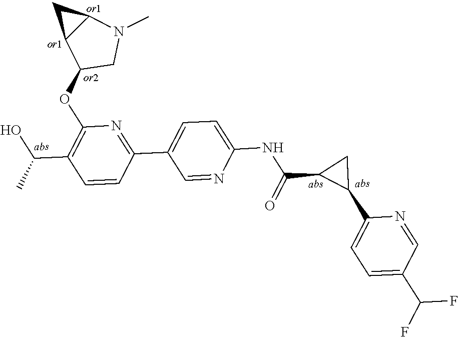 [SMILES: C[C@H](O)c1ccc(-c2ccc(NC(=O)[C@H]3C[C@H]3c3ccc(C(F)F)cn3)nc2)nc1O[C@@H]1CN(C)[C@@H]2C[C@@H]21] | LCMS-2 | 522.2 | 1.35 |
| I-190 | 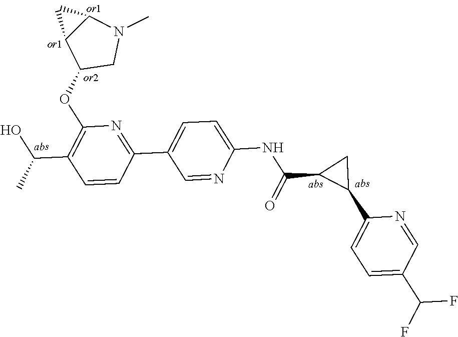 [SMILES: C[C@H](O)c1ccc(-c2ccc(NC(=O)[C@H]3C[C@H]3c3ccc(C(F)F)cn3)nc2)nc1O[C@H]1CN(C)[C@H]2C[C@@H]12] | LCMS-2 | 522.3 | 1.35 |
| I-191 | 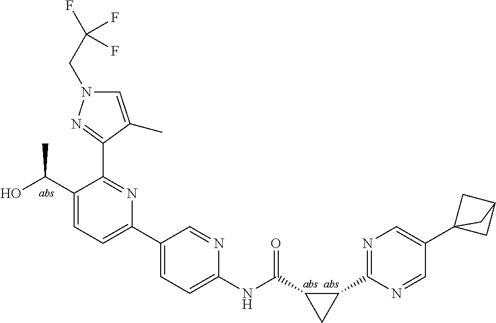 [SMILES: Cc1cn(CC(F)(F)F)nc1-c1nc(-c2ccc(NC(=O)[C@H]3C[C@H]3c3ncc(C45CC(C4)C5)cn3)nc2)ccc1[C@H](C)O] | LCMS-2 | 590.25 | 1.73 |
| I-192 | 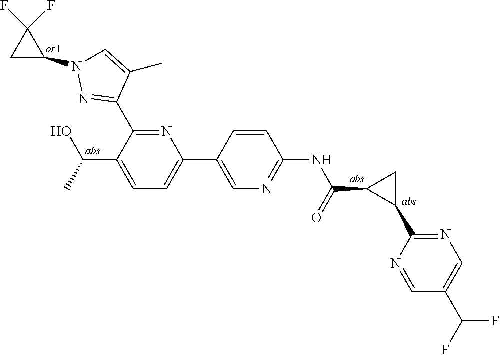 [SMILES: Cc1cn([C@H]2CC2(F)F)nc1-c1nc(-c2ccc(NC(=O)[C@H]3C[C@H]3c3ncc(C(F)F)cn3)nc2)ccc1[C@H](C)O] | LCMS-2 | 568.2 | 1.34 |
| I-193 | 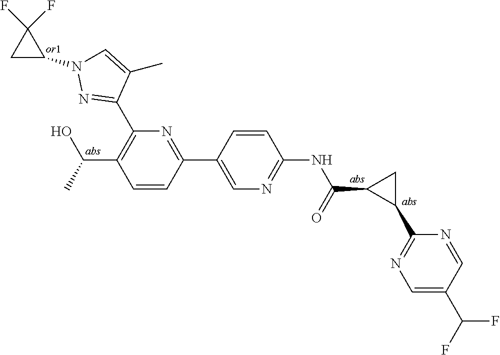 [SMILES: Cc1cn([C@@H]2CC2(F)F)nc1-c1nc(-c2ccc(NC(=O)[C@H]3C[C@H]3c3ncc(C(F)F)cn3)nc2)ccc1[C@H](C)O] | LCMS-2 | 568.25 | 1.33 |
| I-194 | 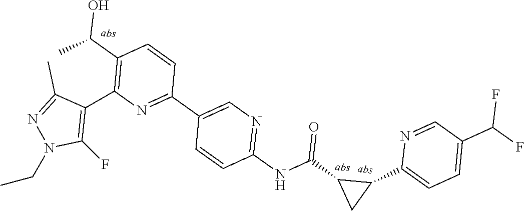 [SMILES: CCn1nc(C)c(-c2nc(-c3ccc(NC(=O)[C@H]4C[C@H]4c4ccc(C(F)F)cn4)nc3)ccc2[C@H](C)O)c1F] | LCMS-2 | 537.25 | 1.2 |
| I-195 | 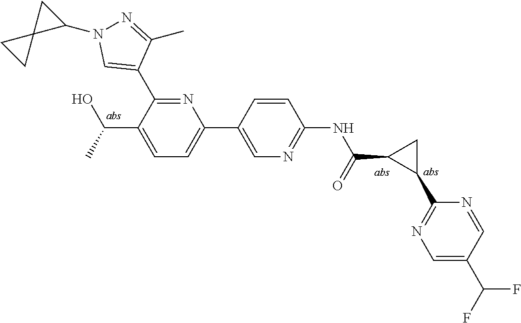 [SMILES: Cc1nn(C2CC23CC3)cc1-c1nc(-c2ccc(NC(=O)[C@H]3C[C@H]3c3ncc(C(F)F)cn3)nc2)ccc1[C@H](C)O] | LCMS-2 | 558.25 | 1.2 |
| I-196 | 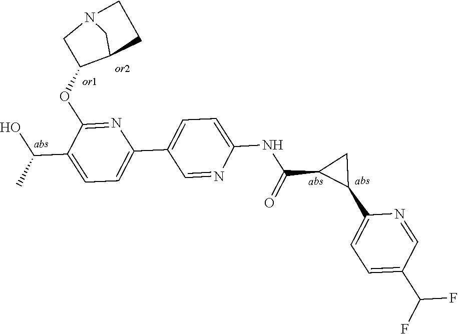 [SMILES: C[C@H](O)c1ccc(-c2ccc(NC(=O)[C@H]3C[C@H]3c3ccc(C(F)F)cn3)nc2)nc1O[C@@H]1CN2CC[C@@H]1C2] | LCMS-2 | 522.2 | 0.79 |
| I-197 | 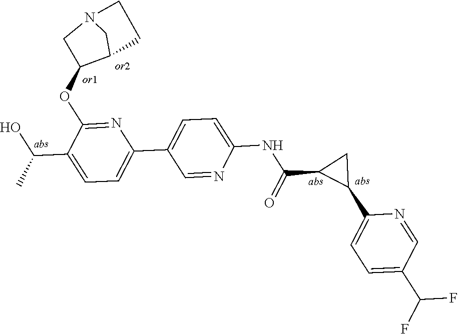 [SMILES: C[C@H](O)c1ccc(-c2ccc(NC(=O)[C@H]3C[C@H]3c3ccc(C(F)F)cn3)nc2)nc1O[C@H]1CN2CC[C@H]1C2] | LCMS-2 | 522.2 | 0.81 |
| I-198 | 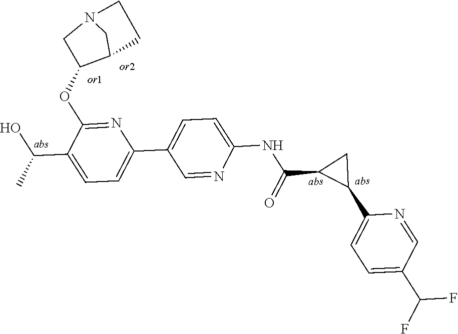 [SMILES: C[C@H](O)c1ccc(-c2ccc(NC(=O)[C@H]3C[C@H]3c3ccc(C(F)F)cn3)nc2)nc1O[C@@H]1CN2CC[C@H]1C2] | LCMS-2 | 522.2 | 0.85 |
| I-199 | 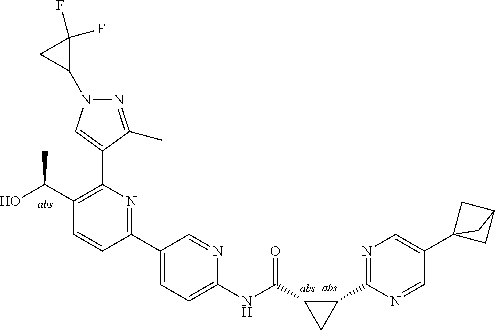 [SMILES: Cc1nn(C2CC2(F)F)cc1-c1nc(-c2ccc(NC(=O)[C@H]3C[C@H]3c3ncc(C45CC(C4)C5)cn3)nc2)ccc1[C@H](C)O] | LCMS-2 | 584.25 | 1.35 |
| I-200 | 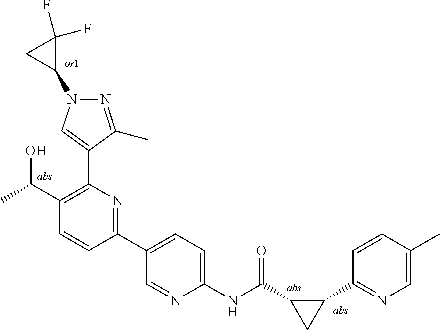 [SMILES: Cc1ccc([C@@H]2C[C@@H]2C(=O)Nc2ccc(-c3ccc([C@H](C)O)c(-c4cn([C@H]5CC5(F)F)nc4C)n3)cn2)nc1] | LCMS-2 | 532.25 | 1.15 |
| I-201 | 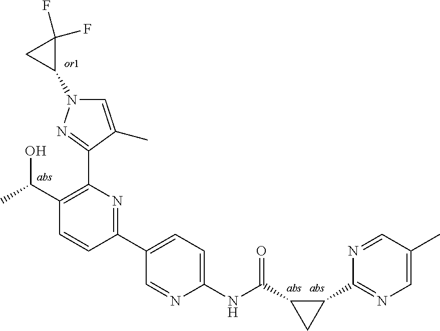 [SMILES: Cc1cnc([C@@H]2C[C@@H]2C(=O)Nc2ccc(-c3ccc([C@H](C)O)c(-c4nn([C@@H]5CC5(F)F)cc4C)n3)cn2)nc1] | LCMS-2 | 532.25 | 1.14 |
| I-202 | 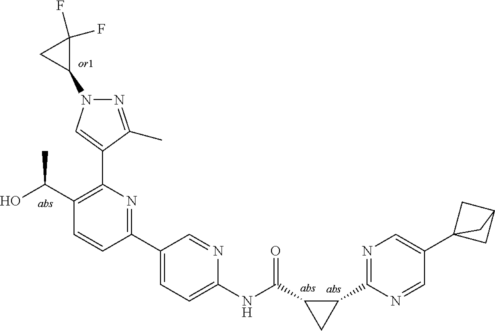 [SMILES: Cc1nn([C@H]2CC2(F)F)cc1-c1nc(-c2ccc(NC(=O)[C@H]3C[C@H]3c3ncc(C45CC(C4)C5)cn3)nc2)ccc1[C@H](C)O] | LCMS-2 | 584.3 | 1.37 |
| I-203 | 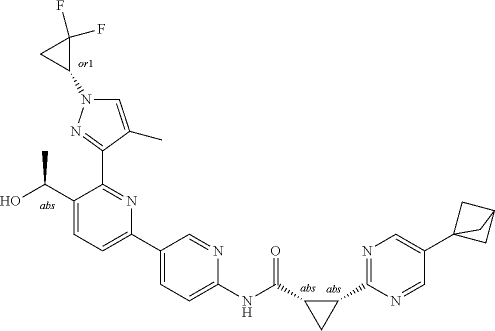 [SMILES: Cc1cn([C@@H]2CC2(F)F)nc1-c1nc(-c2ccc(NC(=O)[C@H]3C[C@H]3c3ncc(C45CC(C4)C5)cn3)nc2)ccc1[C@H](C)O] | LCMS-2 | 584.3 | 1.66 |
| I-204 | 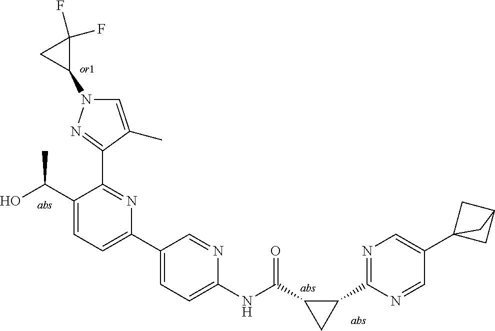 [SMILES: Cc1cn([C@H]2CC2(F)F)nc1-c1nc(-c2ccc(NC(=O)[C@H]3C[C@H]3c3ncc(C45CC(C4)C5)cn3)nc2)ccc1[C@H](C)O] | LCMS-2 | 584.25 | 1.67 |
| I-205 | 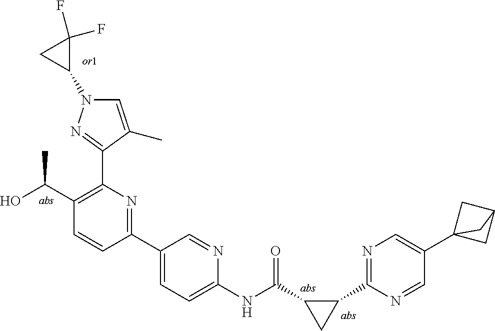 [SMILES: Cc1cn([C@@H]2CC2(F)F)nc1-c1nc(-c2ccc(NC(=O)[C@H]3C[C@H]3c3ncc(C45CC(C4)C5)cn3)nc2)ccc1[C@H](C)O] | LCMS-2 | 584.3 | 1.38 |
| I-206 | 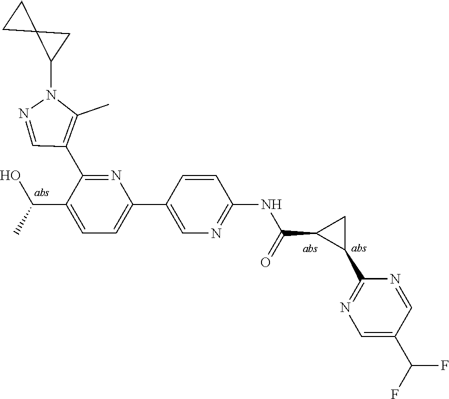 [SMILES: Cc1c(-c2nc(-c3ccc(NC(=O)[C@H]4C[C@H]4c4ncc(C(F)F)cn4)nc3)ccc2[C@H](C)O)cnn1C1CC12CC2] | LCMS-2 | 558.2 | 1.21 |
| I-207 | 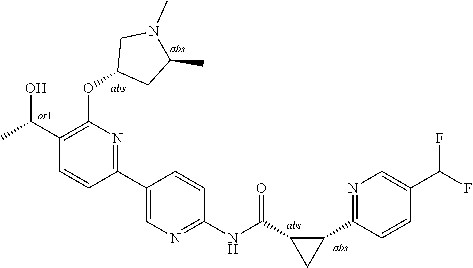 [SMILES: C[C@H](O)c1ccc(-c2ccc(NC(=O)[C@H]3C[C@H]3c3ccc(C(F)F)cn3)nc2)nc1O[C@H]1C[C@H](C)N(C)C1] | LCMS-2 | 524.25 | 1.11 |
| I-208 | 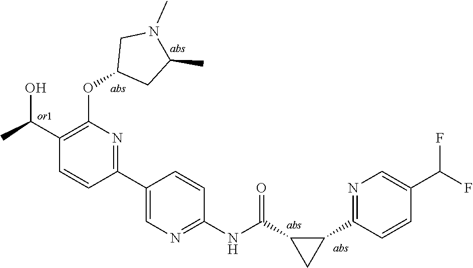 [SMILES: C[C@@H](O)c1ccc(-c2ccc(NC(=O)[C@H]3C[C@H]3c3ccc(C(F)F)cn3)nc2)nc1O[C@H]1C[C@H](C)N(C)C1] | LCMS-2 | 524.2 | 1.1 |
| I-209 | 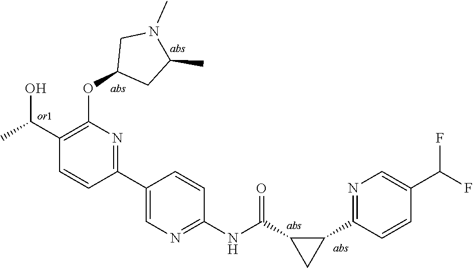 [SMILES: C[C@H](O)c1ccc(-c2ccc(NC(=O)[C@H]3C[C@H]3c3ccc(C(F)F)cn3)nc2)nc1O[C@@H]1C[C@H](C)N(C)C1] | LCMS-2 | 524.3 | 1.39 |
| I-210 | 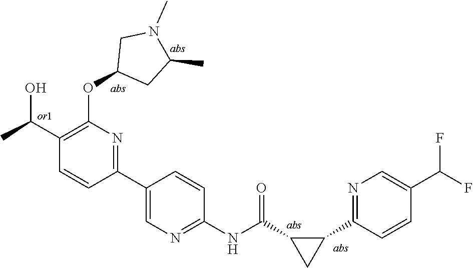 [SMILES: C[C@@H](O)c1ccc(-c2ccc(NC(=O)[C@H]3C[C@H]3c3ccc(C(F)F)cn3)nc2)nc1O[C@@H]1C[C@H](C)N(C)C1] | LCMS-2 | 524.3 | 1.35 |
| I-211 | 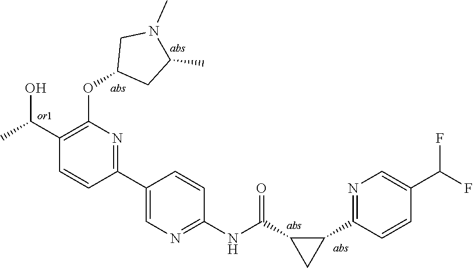 [SMILES: C[C@H](O)c1ccc(-c2ccc(NC(=O)[C@H]3C[C@H]3c3ccc(C(F)F)cn3)nc2)nc1O[C@H]1C[C@@H](C)N(C)C1] | LCMS-2 | 524.3 | 1.33 |
| I-212 | 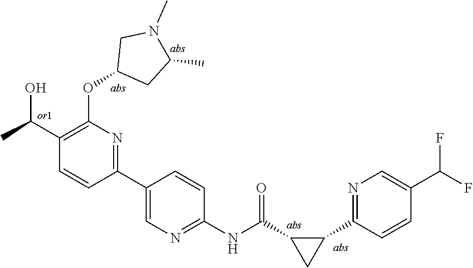 [SMILES: C[C@@H]1C[C@H](Oc2nc(-c3ccc(NC(=O)[C@H]4C[C@H]4c4ccc(C(F)F)cn4)nc3)ccc2[C@@H](C)O)CN1C] | LCMS-2 | 524.25 | 1.4 |
| I-213 | 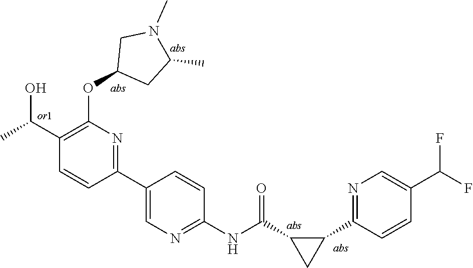 [SMILES: C[C@H](O)c1ccc(-c2ccc(NC(=O)[C@H]3C[C@H]3c3ccc(C(F)F)cn3)nc2)nc1O[C@@H]1C[C@@H](C)N(C)C1] | LCMS-2 | 524.25 | 1.15 |
| I-214 | 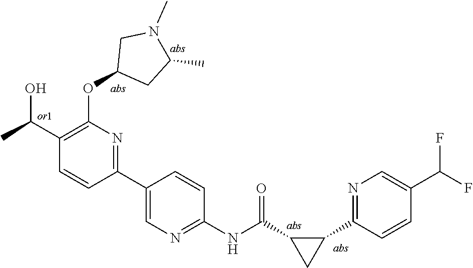 [SMILES: C[C@@H]1C[C@@H](Oc2nc(-c3ccc(NC(=O)[C@H]4C[C@H]4c4ccc(C(F)F)cn4)nc3)ccc2[C@@H](C)O)CN1C] | LCMS-2 | 524.25 | 1.13 |
| I-215 | 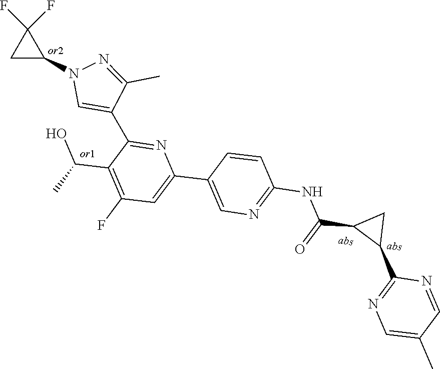 [SMILES: Cc1cnc([C@@H]2C[C@@H]2C(=O)Nc2ccc(-c3cc(F)c([C@H](C)O)c(-c4cn([C@H]5CC5(F)F)nc4C)n3)cn2)nc1] | LCMS-2 | 550.2 | 0.98 |
| I-216 | 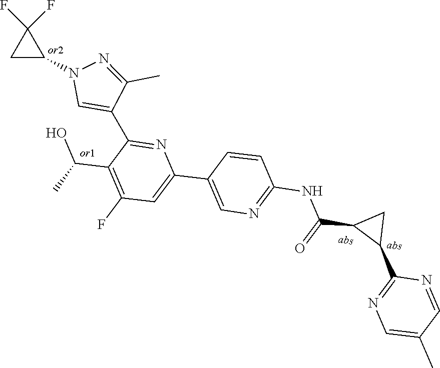 [SMILES: Cc1cnc([C@@H]2C[C@@H]2C(=O)Nc2ccc(-c3cc(F)c([C@H](C)O)c(-c4cn([C@@H]5CC5(F)F)nc4C)n3)cn2)nc1] | LCMS-2 | 550.25 | 0.95 |
| I-217 | 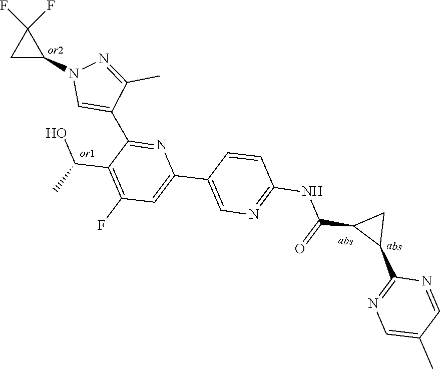 [SMILES: Cc1cnc([C@@H]2C[C@@H]2C(=O)Nc2ccc(-c3cc(F)c([C@H](C)O)c(-c4cn([C@H]5CC5(F)F)nc4C)n3)cn2)nc1] | LCMS-2 | 550.2 | 0.95 |
| I-218 | 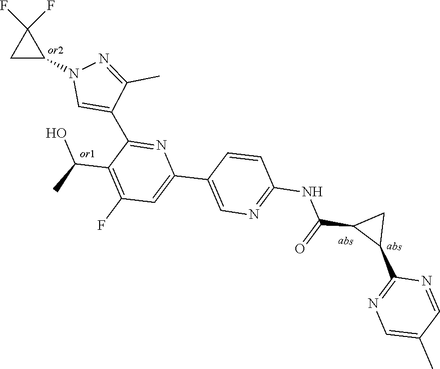 [SMILES: Cc1cnc([C@@H]2C[C@@H]2C(=O)Nc2ccc(-c3cc(F)c([C@@H](C)O)c(-c4cn([C@@H]5CC5(F)F)nc4C)n3)cn2)nc1] | LCMS-2 | 550.25 | 0.95 |
| I-219 | 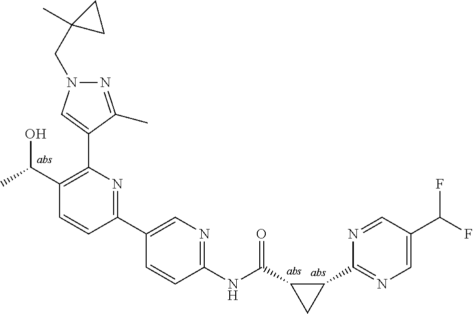 [SMILES: Cc1nn(CC2(C)CC2)cc1-c1nc(-c2ccc(NC(=O)[C@H]3C[C@H]3c3ncc(C(F)F)cn3)nc2)ccc1[C@H](C)O] | LCMS-2 | 560.3 | 1.18 |
| I-220 | 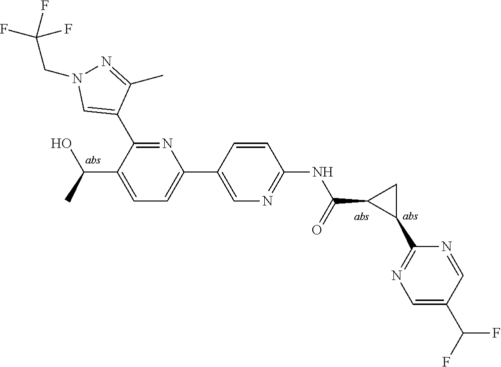 [SMILES: Cc1nn(CC(F)(F)F)cc1-c1nc(-c2ccc(NC(=O)[C@H]3C[C@H]3c3ncc(C(F)F)cn3)nc2)ccc1[C@@H](C)O] | LCMS-2 | 574.3 | 1.09 |
| I-221 | 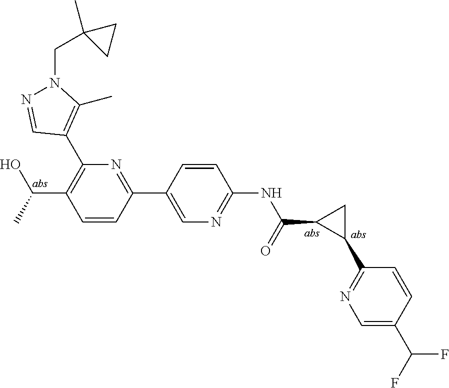 [SMILES: Cc1c(-c2nc(-c3ccc(NC(=O)[C@H]4C[C@H]4c4ccc(C(F)F)cn4)nc3)ccc2[C@H](C)O)cnn1CC1(C)CC1] | LCMS-2 | 560.3 | 1.2 |
| I-222 | 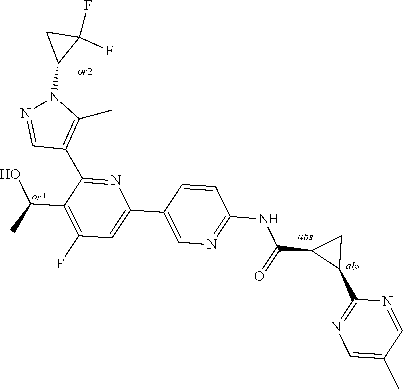 [SMILES: Cc1cnc([C@@H]2C[C@@H]2C(=O)Nc2ccc(-c3cc(F)c([C@@H](C)O)c(-c4cnn([C@@H]5CC5(F)F)c4C)n3)cn2)nc1] | LCMS-2 | 550.25 | 1 |
| I-223 | 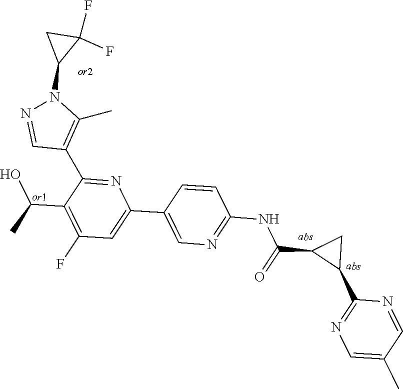 [SMILES: Cc1cnc([C@@H]2C[C@@H]2C(=O)Nc2ccc(-c3cc(F)c([C@@H](C)O)c(-c4cnn([C@H]5CC5(F)F)c4C)n3)cn2)nc1] | LCMS-2 | 550.2 | 1.02 |
| I-224 | 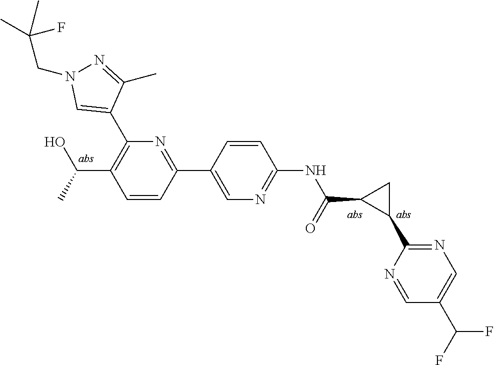 [SMILES: Cc1nn(CC(C)(C)F)cc1-c1nc(-c2ccc(NC(=O)[C@H]3C[C@H]3c3ncc(C(F)F)cn3)nc2)ccc1[C@H](C)O] | LCMS-2 | 566.25 | 1.06 |
| I-225 | 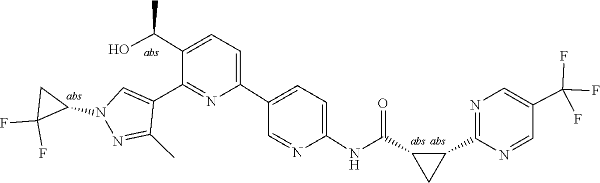 [SMILES: Cc1nn([C@H]2CC2(F)F)cc1-c1nc(-c2ccc(NC(=O)[C@H]3C[C@H]3c3ncc(C(F)(F)F)cn3)nc2)ccc1[C@H](C)O] | LCMS-2 | 586.3 | 1.34 |
| I-226 | 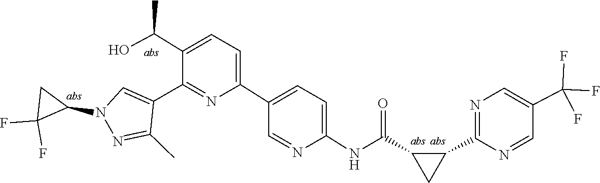 [SMILES: Cc1nn([C@@H]2CC2(F)F)cc1-c1nc(-c2ccc(NC(=O)[C@H]3C[C@H]3c3ncc(C(F)(F)F)cn3)nc2)ccc1[C@H](C)O] | LCMS-2 | 586.2 | 1.35 |
| I-227 | 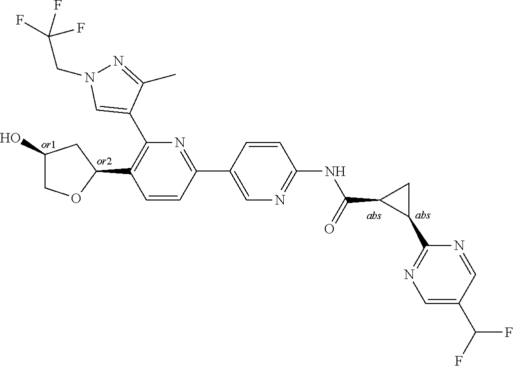 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1C[C@H](O)CO1] | LCMS-2 | 616.2 | 0.97 |
| I-228 | 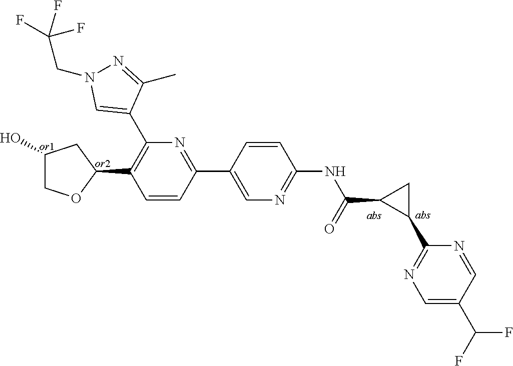 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1C[C@@H](O)CO1] | LCMS-2 | 616.2 | 0.97 |
| I-229 | 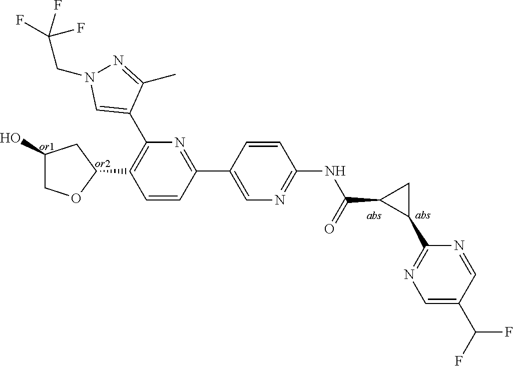 [SMILES: Cc1nn(CC(F)(F)F)cc1-c1nc(-c2ccc(NC(=O)[C@H]3C[C@H]3c3ncc(C(F)F)cn3)nc2)ccc1[C@H]1C[C@H](O)CO1] | LCMS-2 | 616.25 | 0.99 |
| I-230 | 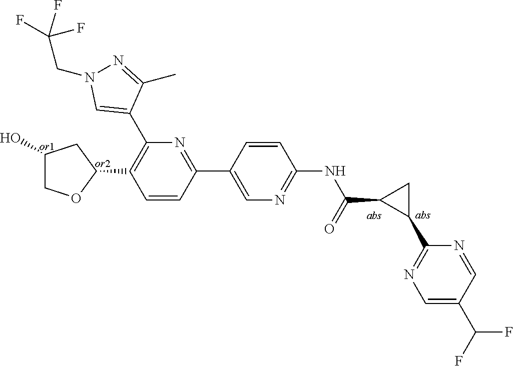 [SMILES: Cc1nn(CC(F)(F)F)cc1-c1nc(-c2ccc(NC(=O)[C@H]3C[C@H]3c3ncc(C(F)F)cn3)nc2)ccc1[C@H]1C[C@@H](O)CO1] | LCMS-2 | 616.25 | 0.99 |
| I-231 | 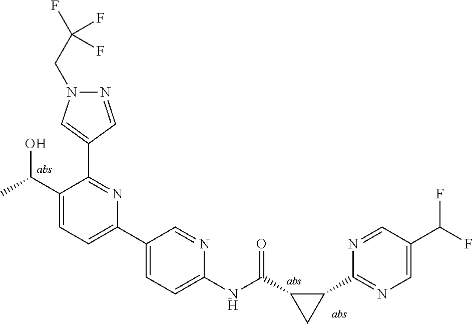 [SMILES: C[C@H](O)c1ccc(-c2ccc(NC(=O)[C@H]3C[C@H]3c3ncc(C(F)F)cn3)nc2)nc1-c1cnn(CC(F)(F)F)c1] | LCMS-2 | 560.15 | 1.11 |
| I-232 | 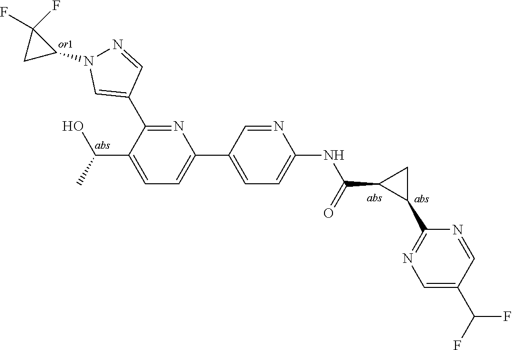 [SMILES: C[C@H](O)c1ccc(-c2ccc(NC(=O)[C@H]3C[C@H]3c3ncc(C(F)F)cn3)nc2)nc1-c1cnn([C@@H]2CC2(F)F)c1] | LCMS-2 | 554.2 | 1.09 |
| I-233 | 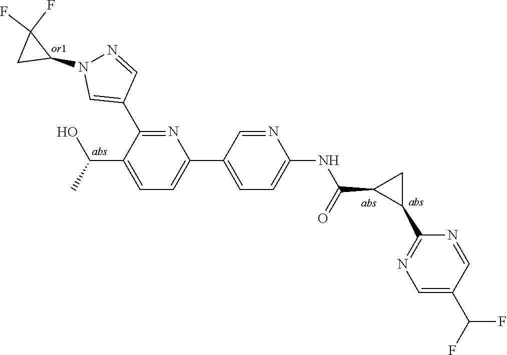 [SMILES: C[C@H](O)c1ccc(-c2ccc(NC(=O)[C@H]3C[C@H]3c3ncc(C(F)F)cn3)nc2)nc1-c1cnn([C@H]2CC2(F)F)c1] | LCMS-2 | 554.2 | 1.09 |
| I-234 | 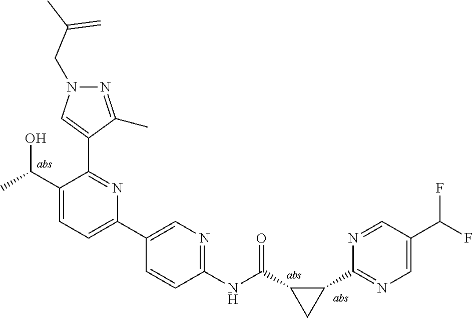 [SMILES: C=C(C)Cn1cc(-c2nc(-c3ccc(NC(=O)[C@H]4C[C@H]4c4ncc(C(F)F)cn4)nc3)ccc2[C@H](C)O)c(C)n1] | LCMS-2 | 546.25 | 1.06 |
| I-235 | 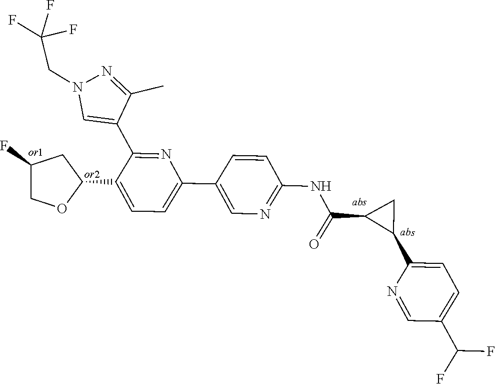 [SMILES: Cc1nn(CC(F)(F)F)cc1-c1nc(-c2ccc(NC(=O)[C@H]3C[C@H]3c3ccc(C(F)F)cn3)nc2)ccc1[C@H]1C[C@H](F)CO1] | LCMS-2 | 617.25 | 1.58 |
| I-236 | 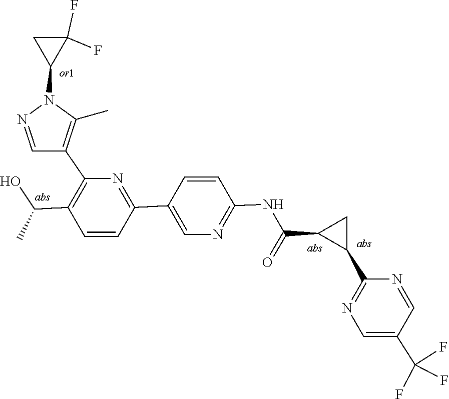 [SMILES: Cc1c(-c2nc(-c3ccc(NC(=O)[C@H]4C[C@H]4c4ncc(C(F)(F)F)cn4)nc3)ccc2[C@H](C)O)cnn1[C@H]1CC1(F)F] | LCMS-2 | 586.25 | 1.28 |
| I-237 | 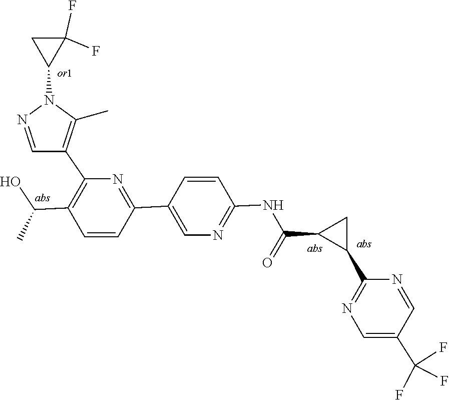 [SMILES: Cc1c(-c2nc(-c3ccc(NC(=O)[C@H]4C[C@H]4c4ncc(C(F)(F)F)cn4)nc3)ccc2[C@H](C)O)cnn1[C@@H]1CC1(F)F] | LCMS-2 | 586.25 | 1.31 |
| I-238 | 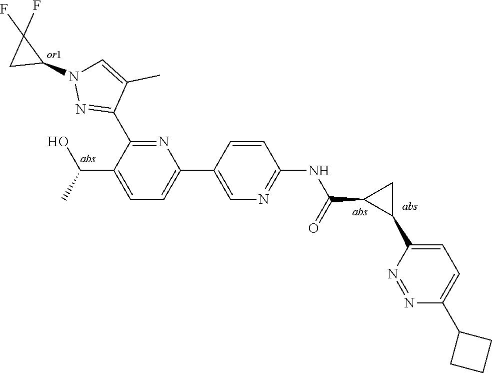 [SMILES: Cc1cn([C@H]2CC2(F)F)nc1-c1nc(-c2ccc(NC(=O)[C@H]3C[C@H]3c3ccc(C4CCC4)nn3)nc2)ccc1[C@H](C)O] | LCMS-2 | 572.25 | 1.56 |
| I-239 | 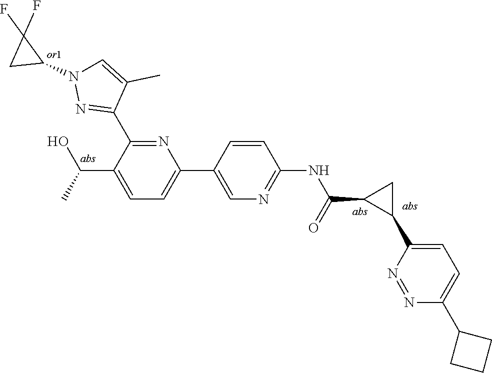 [SMILES: Cc1cn([C@@H]2CC2(F)F)nc1-c1nc(-c2ccc(NC(=O)[C@H]3C[C@H]3c3ccc(C4CCC4)nn3)nc2)ccc1[C@H](C)O] | LCMS-2 | 572.3 | 1.54 |
| I-240 | 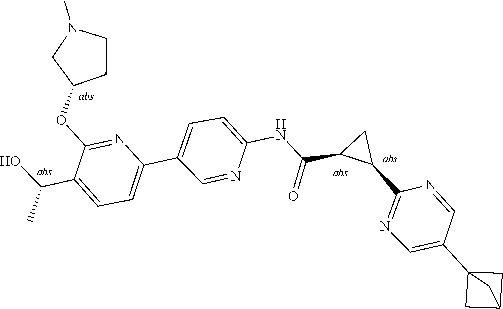 [SMILES: C[C@H](O)c1ccc(-c2ccc(NC(=O)[C@H]3C[C@H]3c3ncc(C45CC(C4)C5)cn3)nc2)nc1O[C@H]1CCN(C)C1] | LCMS-2 | 527.3 | 1.18 |
| I-241 | 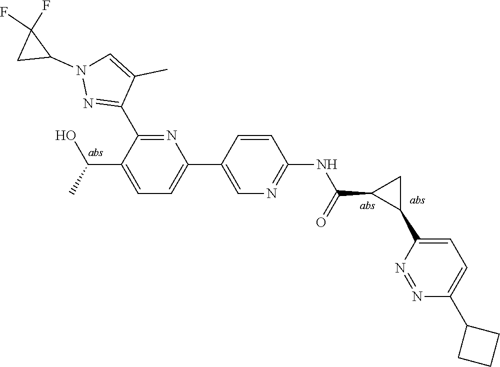 [SMILES: Cc1cn(C2CC2(F)F)nc1-c1nc(-c2ccc(NC(=O)[C@H]3C[C@H]3c3ccc(C4CCC4)nn3)nc2)ccc1[C@H](C)O] | LCMS-2 | 572.3 | 1.49 |
| I-242 | 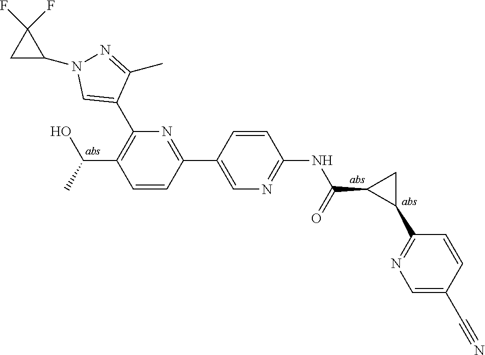 [SMILES: Cc1nn(C2CC2(F)F)cc1-c1nc(-c2ccc(NC(=O)[C@H]3C[C@H]3c3ccc(C#N)cn3)nc2)ccc1[C@H](C)O] | LCMS-2 | 542.25 | 1.4 |
| I-243 | 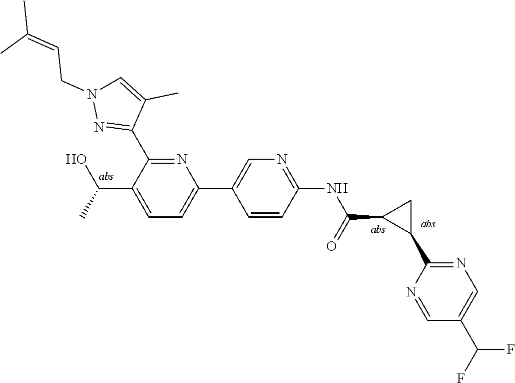 [SMILES: CC(C)=CCn1cc(C)c(-c2nc(-c3ccc(NC(=O)[C@H]4C[C@H]4c4ncc(C(F)F)cn4)nc3)ccc2[C@H](C)O)n1] | LCMS-2 | 560.3 | 1.24 |
| I-244 | 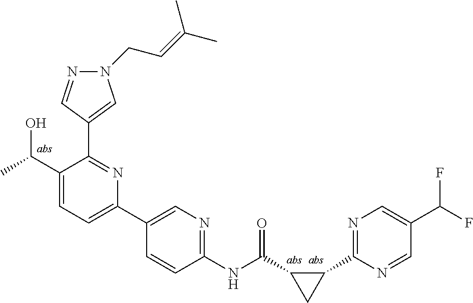 | LCMS-2 | 560.25 | 1.27 |
| I-245 | 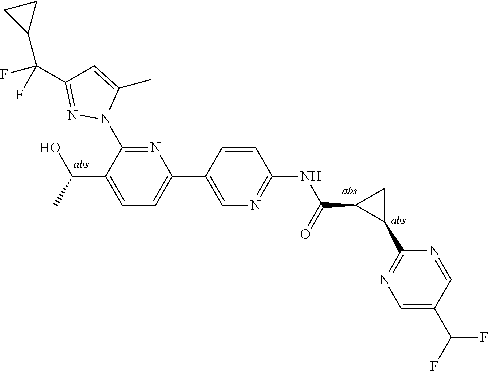 [SMILES: Cc1cc(C(F)(F)C2CC2)nn1-c1nc(-c2ccc(NC(=O)[C@H]3C[C@H]3c3ncc(C(F)F)cn3)nc2)ccc1[C@H](C)O] | LCMS-2 | 582.25 | 1.6 |
| I-246 | 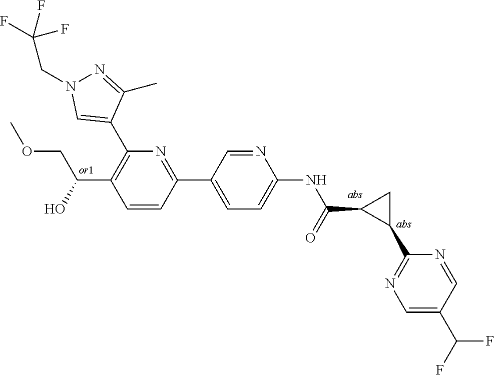 [SMILES: COC[C@@H](O)c1ccc(-c2ccc(NC(=O)[C@H]3C[C@H]3c3ncc(C(F)F)cn3)nc2)nc1-c1cn(CC(F)(F)F)nc1C] | LCMS-2 | 604.25 | 1.03 |
| I-247 | 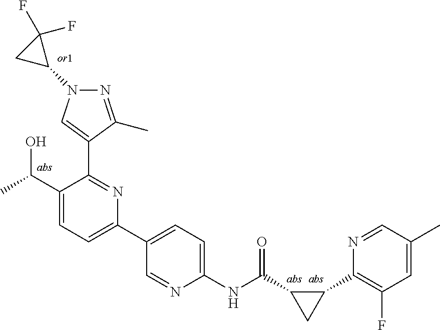 [SMILES: Cc1cnc([C@@H]2C[C@@H]2C(=O)Nc2ccc(-c3ccc([C@H](C)O)c(-c4cn([C@@H]5CC5(F)F)nc4C)n3)cn2)c(F)c1] | LCMS-2 | 549.25 | 1.26 |
| I-248 | 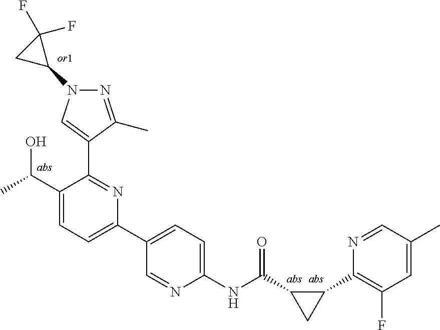 [SMILES: Cc1cnc([C@@H]2C[C@@H]2C(=O)Nc2ccc(-c3ccc([C@H](C)O)c(-c4cn([C@H]5CC5(F)F)nc4C)n3)cn2)c(F)c1] | LCMS-2 | 549.25 | 1.24 |
| I-249 | 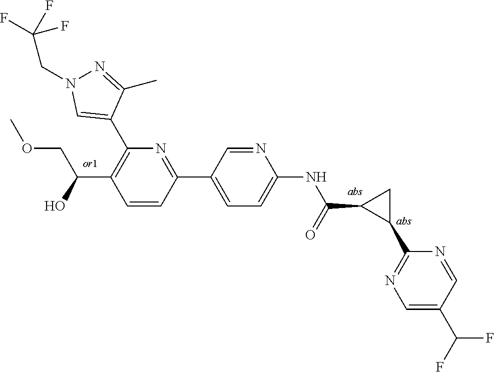 [SMILES: COC[C@H](O)c1ccc(-c2ccc(NC(=O)[C@H]3C[C@H]3c3ncc(C(F)F)cn3)nc2)nc1-c1cn(CC(F)(F)F)nc1C] | LCMS-2 | 604.2 | 1.03 |
| I-250 | 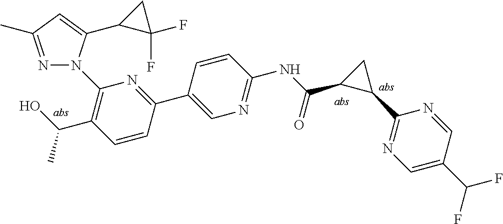 [SMILES: Cc1cc(C2CC2(F)F)n(-c2nc(-c3ccc(NC(=O)[C@H]4C[C@H]4c4ncc(C(F)F)cn4)nc3)ccc2[C@H](C)O)n1] | LCMS-2 | 568.25 | 1.36 |
| I-251 | 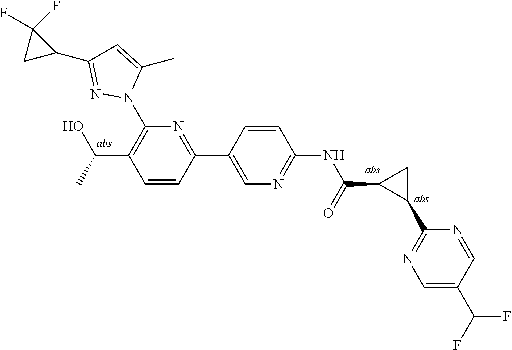 [SMILES: Cc1cc(C2CC2(F)F)nn1-c1nc(-c2ccc(NC(=O)[C@H]3C[C@H]3c3ncc(C(F)F)cn3)nc2)ccc1[C@H](C)O] | LCMS-2 | 568.25 | 1.41 |
| I-252 | 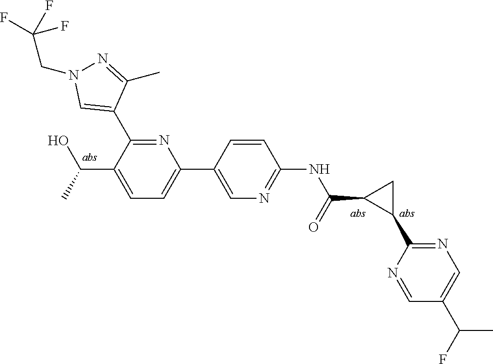 [SMILES: Cc1nn(CC(F)(F)F)cc1-c1nc(-c2ccc(NC(=O)[C@H]3C[C@H]3c3ncc(C(C)F)cn3)nc2)ccc1[C@H](C)O] | LCMS-2 | 570.25 | 1.05 |
| I-253 | 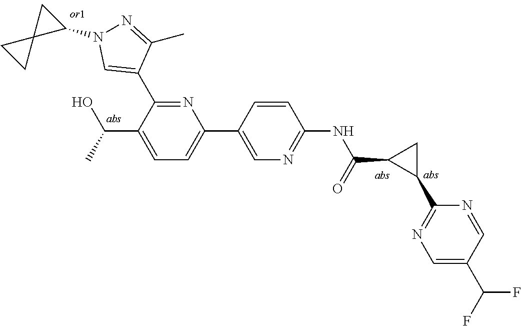 [SMILES: Cc1nn([C@H]2CC23CC3)cc1-c1nc(-c2ccc(NC(=O)[C@H]3C[C@H]3c3ncc(C(F)F)cn3)nc2)ccc1[C@H](C)O] | LCMS-2 | 558.3 | 1.15 |
| I-254 | 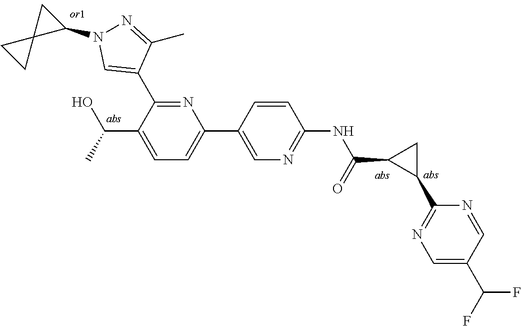 [SMILES: Cc1nn([C@@H]2CC23CC3)cc1-c1nc(-c2ccc(NC(=O)[C@H]3C[C@H]3c3ncc(C(F)F)cn3)nc2)ccc1[C@H](C)O] | LCMS-2 | 558.25 | 1.14 |
| I-255 | 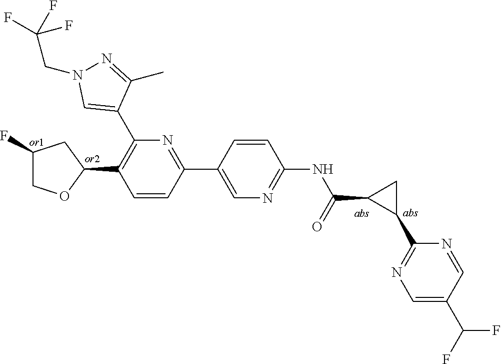 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1C[C@H](F)CO1] | LCMS-2 | 618.15 | 1.43 |
| I-256 | 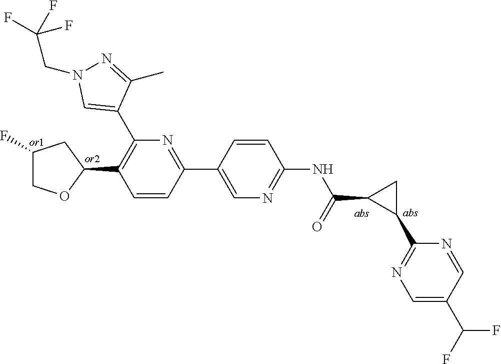 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1C[C@@H](F)CO1] | LCMS-2 | 618.2 | 1.44 |
| I-257 | 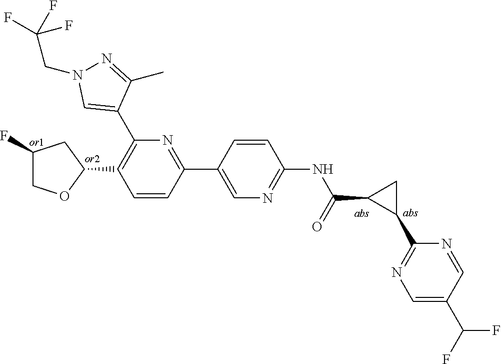 [SMILES: Cc1nn(CC(F)(F)F)cc1-c1nc(-c2ccc(NC(=O)[C@H]3C[C@H]3c3ncc(C(F)F)cn3)nc2)ccc1[C@H]1C[C@H](F)CO1] | LCMS-2 | 618.25 | 1.42 |
| I-258 | 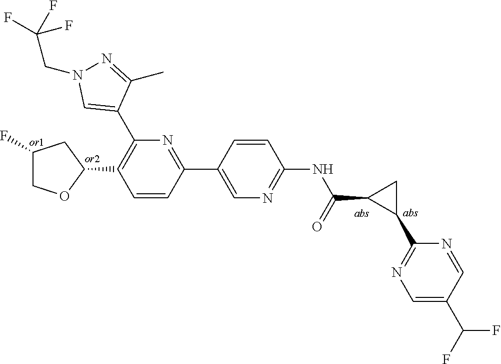 [SMILES: Cc1nn(CC(F)(F)F)cc1-c1nc(-c2ccc(NC(=O)[C@H]3C[C@H]3c3ncc(C(F)F)cn3)nc2)ccc1[C@H]1C[C@@H](F)CO1] | LCMS-2 | 618.25 | 1.41 |
| I-259 | 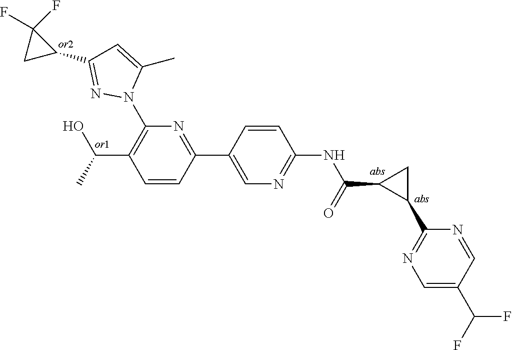 [SMILES: Cc1cc([C@@H]2CC2(F)F)nn1-c1nc(-c2ccc(NC(=O)[C@H]3C[C@H]3c3ncc(C(F)F)cn3)nc2)ccc1[C@H](C)O] | LCMS-2 | 568.25 | 1.41 |
| I-260 | 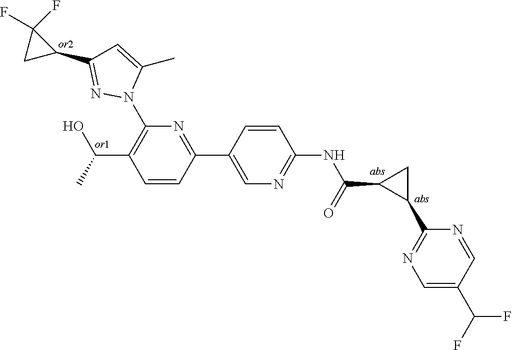 [SMILES: Cc1cc([C@H]2CC2(F)F)nn1-c1nc(-c2ccc(NC(=O)[C@H]3C[C@H]3c3ncc(C(F)F)cn3)nc2)ccc1[C@H](C)O] | LCMS-2 | 568.25 | 1.41 |
| I-261 | 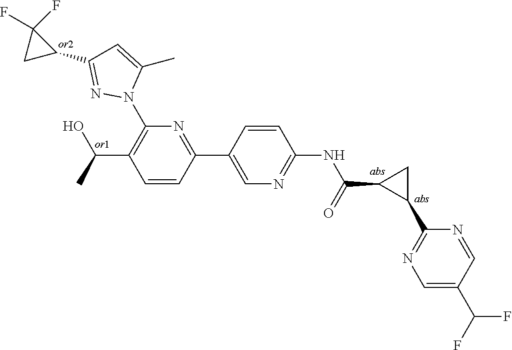 [SMILES: Cc1cc([C@@H]2CC2(F)F)nn1-c1nc(-c2ccc(NC(=O)[C@H]3C[C@H]3c3ncc(C(F)F)cn3)nc2)ccc1[C@@H](C)O] | LCMS-2 | 568.25 | 1.41 |
| I-262 | 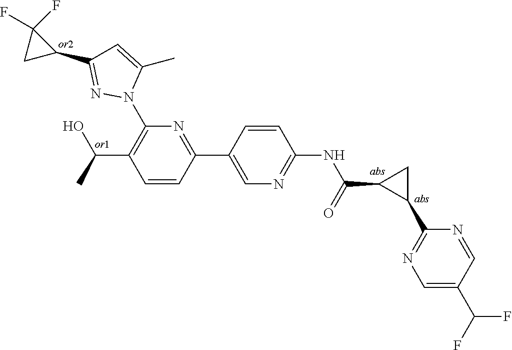 [SMILES: Cc1cc([C@H]2CC2(F)F)nn1-c1nc(-c2ccc(NC(=O)[C@H]3C[C@H]3c3ncc(C(F)F)cn3)nc2)ccc1[C@@H](C)O] | LCMS-2 | 568.2 | 1.41 |
| I-263 | 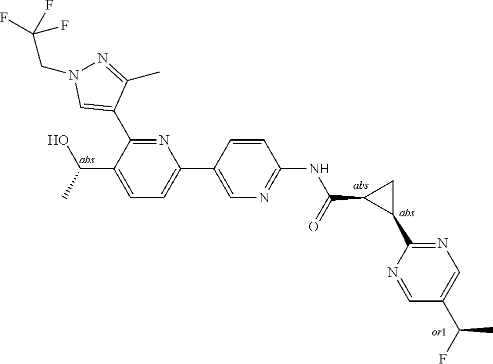 [SMILES: Cc1nn(CC(F)(F)F)cc1-c1nc(-c2ccc(NC(=O)[C@H]3C[C@H]3c3ncc([C@@H](C)F)cn3)nc2)ccc1[C@H](C)O] | LCMS-2 | 570.25 | 1.07 |
| I-264 | 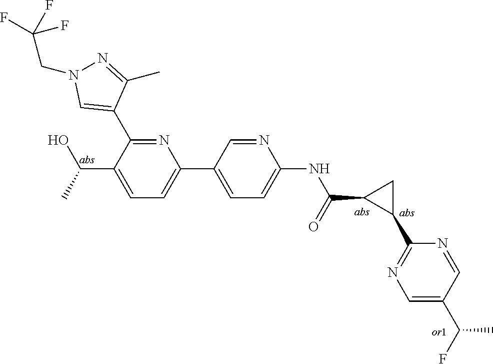 [SMILES: Cc1nn(CC(F)(F)F)cc1-c1nc(-c2ccc(NC(=O)[C@H]3C[C@H]3c3ncc([C@H](C)F)cn3)nc2)ccc1[C@H](C)O] | LCMS-2 | 570.25 | 1.08 |
| I-265 | 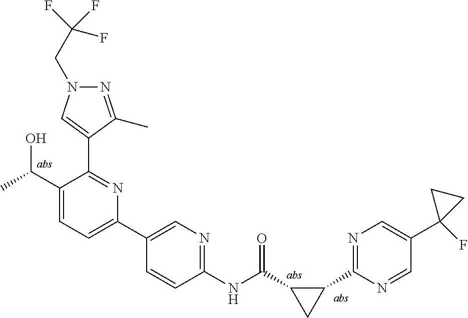 [SMILES: Cc1nn(CC(F)(F)F)cc1-c1nc(-c2ccc(NC(=O)[C@H]3C[C@H]3c3ncc(C4(F)CC4)cn3)nc2)ccc1[C@H](C)O] | LCMS-2 | 582.25 | 1.19 |
| I-266 | 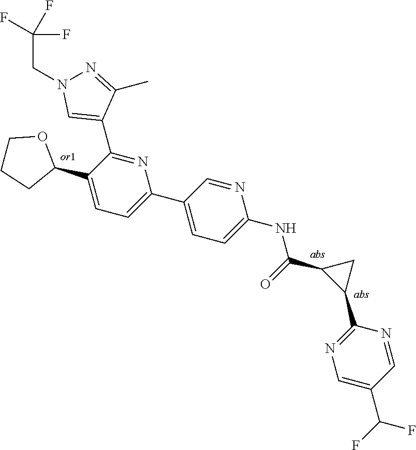 [SMILES: Cc1nn(CC(F)(F)F)cc1-c1nc(-c2ccc(NC(=O)[C@H]3C[C@H]3c3ncc(C(F)F)cn3)nc2)ccc1[C@H]1CCCO1] | LCMS-2 | 600.2 | 1.51 |
| I-267 | 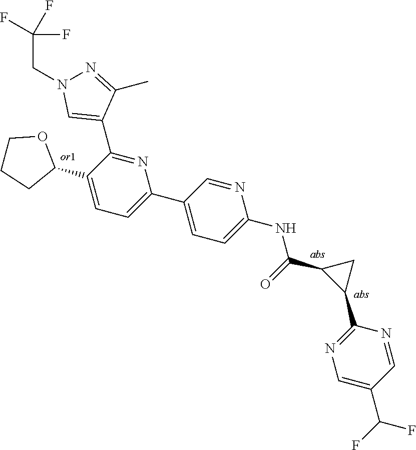 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1CCCO1] | LCMS-2 | 600.2 | 1.51 |
| I-268 | 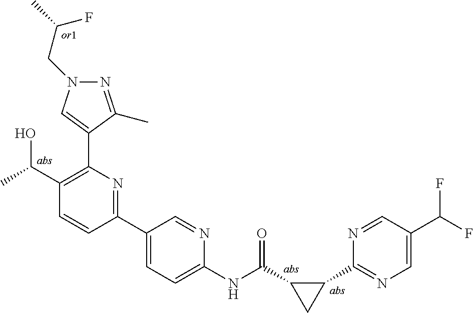 [SMILES: Cc1nn(C[C@H](C)F)cc1-c1nc(-c2ccc(NC(=O)[C@H]3C[C@H]3c3ncc(C(F)F)cn3)nc2)ccc1[C@H](C)O] | LCMS-2 | 552.25 | 0.99 |
| I-269 | 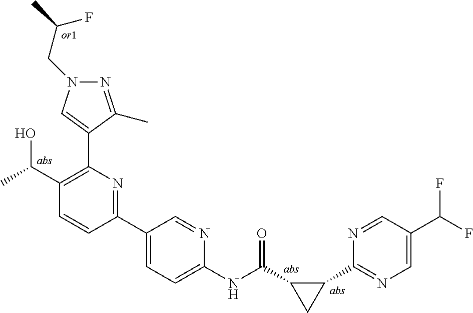 [SMILES: Cc1nn(C[C@@H](C)F)cc1-c1nc(-c2ccc(NC(=O)[C@H]3C[C@H]3c3ncc(C(F)F)cn3)nc2)ccc1[C@H](C)O] | LCMS-2 | 552.25 | 0.99 |
| I-270 | 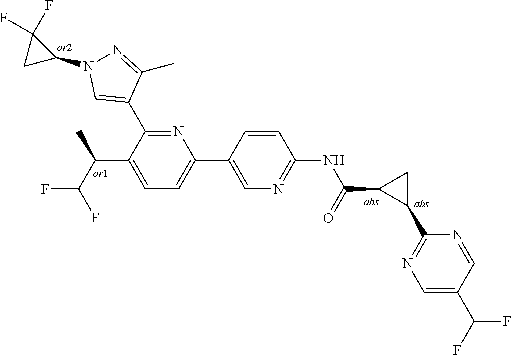 [SMILES: Cc1nn([C@H]2CC2(F)F)cc1-c1nc(-c2ccc(NC(=O)[C@H]3C[C@H]3c3ncc(C(F)F)cn3)nc2)ccc1[C@H](C)C(F)F] | LCMS-2 | 602.25 | 1.59 |
| I-271 | 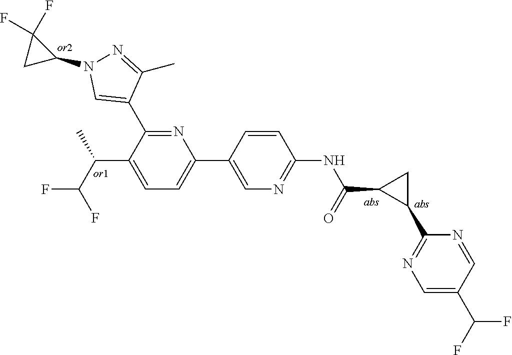 [SMILES: Cc1nn([C@H]2CC2(F)F)cc1-c1nc(-c2ccc(NC(=O)[C@H]3C[C@H]3c3ncc(C(F)F)cn3)nc2)ccc1[C@@H](C)C(F)F] | LCMS-2 | 602.2 | 1.59 |
| I-272 | 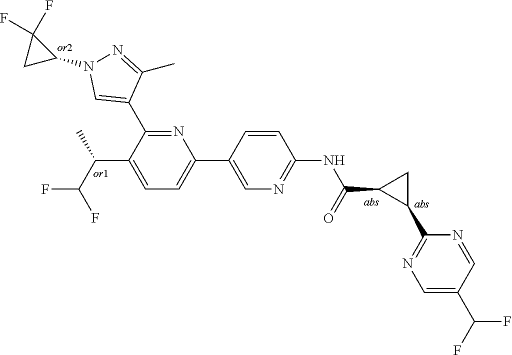 [SMILES: Cc1nn([C@@H]2CC2(F)F)cc1-c1nc(-c2ccc(NC(=O)[C@H]3C[C@H]3c3ncc(C(F)F)cn3)nc2)ccc1[C@@H](C)C(F)F] | LCMS-2 | 602.25 | 1.59 |
| I-273 | 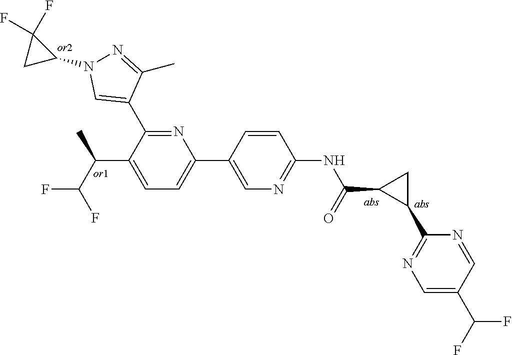 [SMILES: Cc1nn([C@@H]2CC2(F)F)cc1-c1nc(-c2ccc(NC(=O)[C@H]3C[C@H]3c3ncc(C(F)F)cn3)nc2)ccc1[C@H](C)C(F)F] | LCMS-2 | 602.25 | 1.6 |
| I-274 | 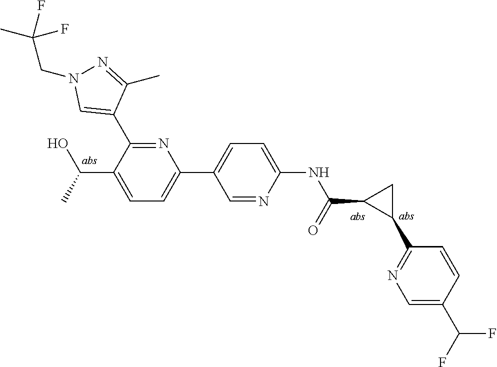 [SMILES: Cc1nn(CC(C)(F)F)cc1-c1nc(-c2ccc(NC(=O)[C@H]3C[C@H]3c3ccc(C(F)F)cn3)nc2)ccc1[C@H](C)O] | LCMS-2 | 569.25 | 1.25 |
| I-275 | 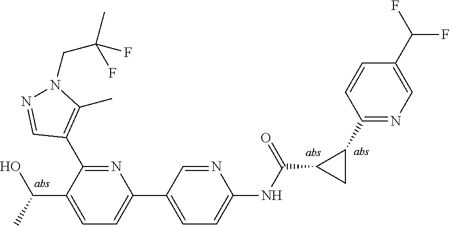 [SMILES: Cc1c(-c2nc(-c3ccc(NC(=O)[C@H]4C[C@H]4c4ccc(C(F)F)cn4)nc3)ccc2[C@H](C)O)cnn1CC(C)(F)F] | LCMS-2 | 569.2 | 1.26 |
| I-276 | 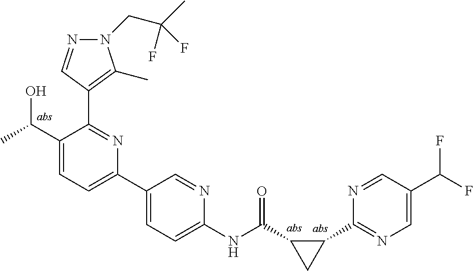 [SMILES: Cc1c(-c2nc(-c3ccc(NC(=O)[C@H]4C[C@H]4c4ncc(C(F)F)cn4)nc3)ccc2[C@H](C)O)cnn1CC(C)(F)F] | LCMS-2 | 570.2 | 1.08 |
| I-277 | 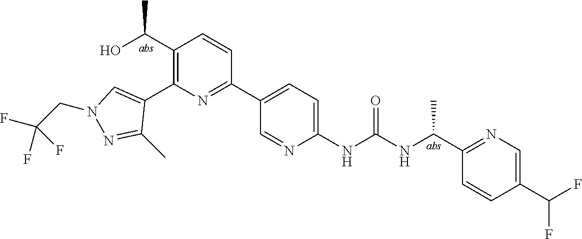 [SMILES: Cc1nn(CC(F)(F)F)cc1-c1nc(-c2ccc(NC(=O)N[C@H](C)c3ccc(C(F)F)cn3)nc2)ccc1[C@H](C)O] | LCMS-2 | 576.2 | 1.35 |
| I-278 | 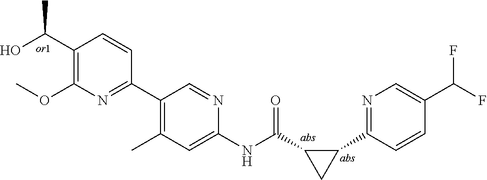 [SMILES: COc1nc(-c2cnc(NC(=O)[C@H]3C[C@H]3c3ccc(C(F)F)cn3)cc2C)ccc1[C@H](C)O] | LCMS-2 | 455.15 | 1.29 |
| I-279 | 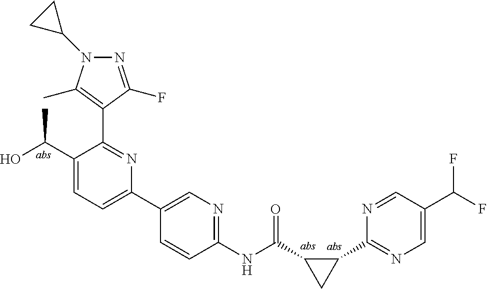 [SMILES: Cc1c(-c2nc(-c3ccc(NC(=O)[C@H]4C[C@H]4c4ncc(C(F)F)cn4)nc3)ccc2[C@H](C)O)c(F)nn1C1CC1] | LCMS-2 | 550.25 | 1.13 |
| I-280 | 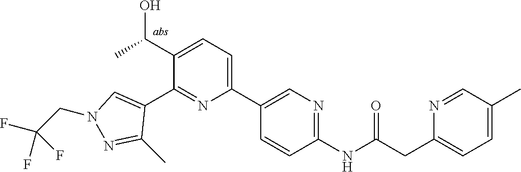 [SMILES: Cc1ccc(CC(=O)Nc2ccc(-c3ccc([C@H](C)O)c(-c4cn(CC(F)(F)F)nc4C)n3)cn2)nc1] | LCMS-2 | 511.15 | 1.23 |
| I-281 | 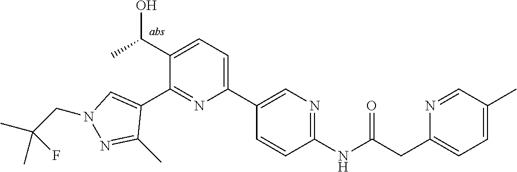 [SMILES: Cc1ccc(CC(=O)Nc2ccc(-c3ccc([C@H](C)O)c(-c4cn(CC(C)(C)F)nc4C)n3)cn2)nc1] | LCMS-2 | 503.25 | 1.2 |
| I-282 | 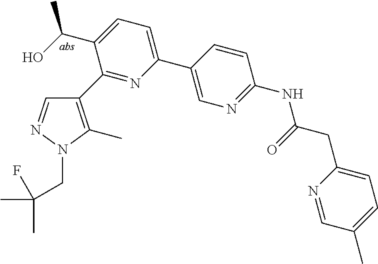 [SMILES: Cc1ccc(CC(=O)Nc2ccc(-c3ccc([C@H](C)O)c(-c4cnn(CC(C)(C)F)c4C)n3)cn2)nc1] | LCMS-2 | 503.25 | 1.22 |
| I-283 | 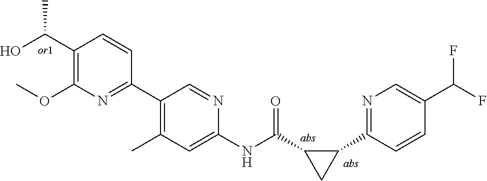 [SMILES: COc1nc(-c2cnc(NC(=O)[C@H]3C[C@H]3c3ccc(C(F)F)cn3)cc2C)ccc1[C@@H](C)O] | LCMS-2 | 455.15 | 1.29 |
| I-284 | 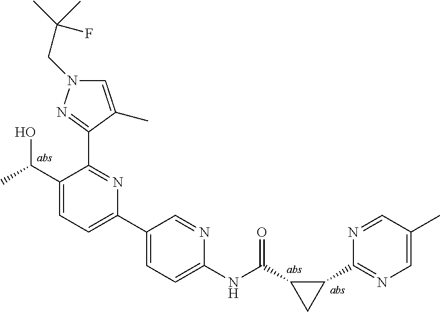 [SMILES: Cc1cnc([C@@H]2C[C@@H]2C(=O)Nc2ccc(-c3ccc([C@H](C)O)c(-c4nn(CC(C)(C)F)cc4C)n3)cn2)nc1] | LCMS-2 | 530.3 | 1.23 |
| I-285 | 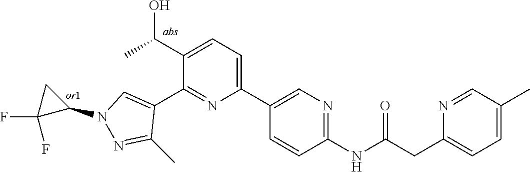 [SMILES: Cc1ccc(CC(=O)Nc2ccc(-c3ccc([C@H](C)O)c(-c4cn([C@@H]5CC5(F)F)nc4C)n3)cn2)nc1] | LCMS-2 | 505.2 | 1.17 |
| I-286 | 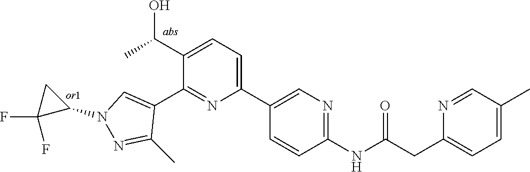 [SMILES: Cc1ccc(CC(=O)Nc2ccc(-c3ccc([C@H](C)O)c(-c4cn([C@H]5CC5(F)F)nc4C)n3)cn2)nc1] | LCMS-2 | 505.2 | 1.2 |
| I-287 | 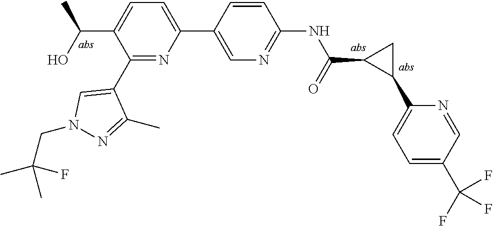 [SMILES: Cc1nn(CC(C)(C)F)cc1-c1nc(-c2ccc(NC(=O)[C@H]3C[C@H]3c3ccc(C(F)(F)F)cn3)nc2)ccc1[C@H](C)O] | LCMS-2 | 583.25 | 0.83 |
| I-288 | 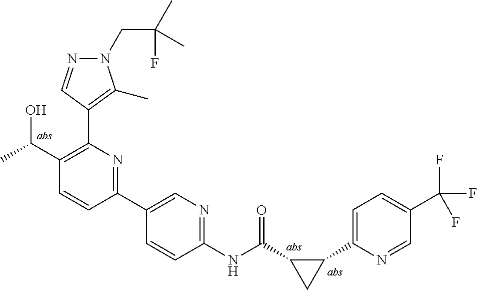 [SMILES: Cc1c(-c2nc(-c3ccc(NC(=O)[C@H]4C[C@H]4c4ccc(C(F)(F)F)cn4)nc3)ccc2[C@H](C)O)cnn1CC(C)(C)F] | LCMS-2 | 583.3 | 1.45 |
| I-289 | 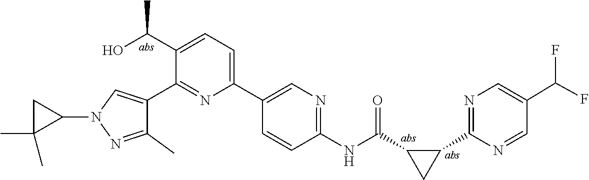 [SMILES: Cc1nn(C2CC2(C)C)cc1-c1nc(-c2ccc(NC(=O)[C@H]3C[C@H]3c3ncc(C(F)F)cn3)nc2)ccc1[C@H](C)O] | LCMS-2 | 560.3 | 1.18 |
| I-290 | 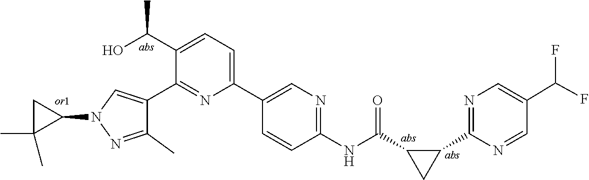 [SMILES: Cc1nn([C@@H]2CC2(C)C)cc1-c1nc(-c2ccc(NC(=O)[C@H]3C[C@H]3c3ncc(C(F)F)cn3)nc2)ccc1[C@H](C)O] | LCMS-2 | 560.25 | 1.21 |
| I-291 | 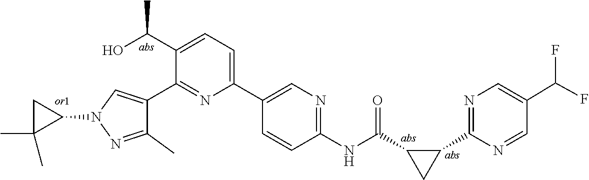 [SMILES: Cc1nn([C@H]2CC2(C)C)cc1-c1nc(-c2ccc(NC(=O)[C@H]3C[C@H]3c3ncc(C(F)F)cn3)nc2)ccc1[C@H](C)O] | LCMS-2 | 560.25 | 1.22 |
| I-292 | 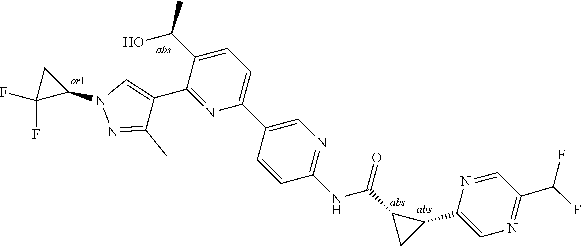 [SMILES: Cc1nn([C@@H]2CC2(F)F)cc1-c1nc(-c2ccc(NC(=O)[C@H]3C[C@H]3c3cnc(C(F)F)cn3)nc2)ccc1[C@H](C)O] | LCMS-2 | 568.2 | 1.35 |
| I-293 | 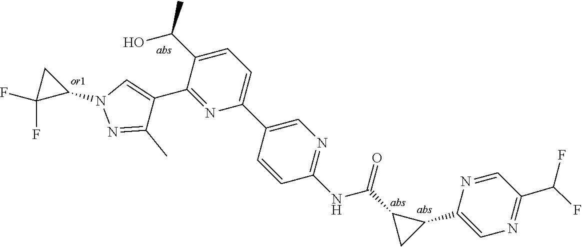 [SMILES: Cc1nn([C@H]2CC2(F)F)cc1-c1nc(-c2ccc(NC(=O)[C@H]3C[C@H]3c3cnc(C(F)F)cn3)nc2)ccc1[C@H](C)O] | LCMS-2 | 568.2 | 1.35 |
| I-294 | 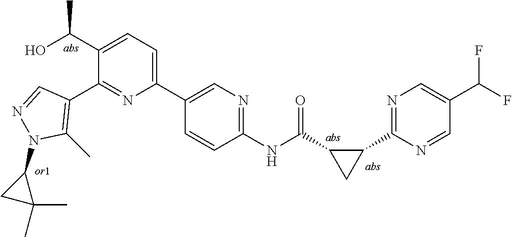 [SMILES: Cc1c(-c2nc(-c3ccc(NC(=O)[C@H]4C[C@H]4c4ncc(C(F)F)cn4)nc3)ccc2[C@H](C)O)cnn1[C@@H]1CC1(C)C] | LCMS-2 | 560.25 | 1.26 |
| I-295 | 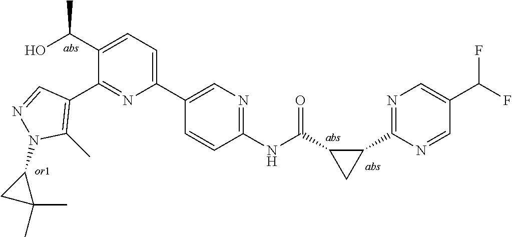 [SMILES: Cc1c(-c2nc(-c3ccc(NC(=O)[C@H]4C[C@H]4c4ncc(C(F)F)cn4)nc3)ccc2[C@H](C)O)cnn1[C@H]1CC1(C)C] | LCMS-2 | 560.25 | 1.23 |
| I-296 | 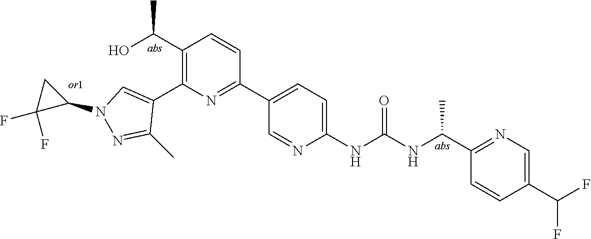 [SMILES: Cc1nn([C@@H]2CC2(F)F)cc1-c1nc(-c2ccc(NC(=O)N[C@H](C)c3ccc(C(F)F)cn3)nc2)ccc1[C@H](C)O] | LCMS-2 | 570.25 | 1.29 |
| I-297 | 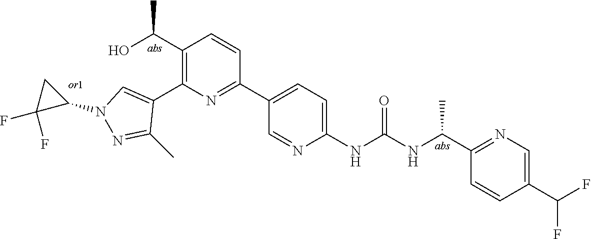 [SMILES: Cc1nn([C@H]2CC2(F)F)cc1-c1nc(-c2ccc(NC(=O)N[C@H](C)c3ccc(C(F)F)cn3)nc2)ccc1[C@H](C)O] | LCMS-2 | 570.2 | 1.31 |
| I-298 | 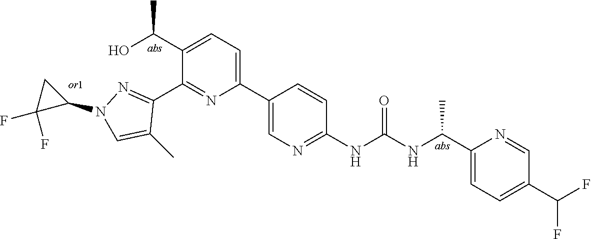 [SMILES: Cc1cn([C@@H]2CC2(F)F)nc1-c1nc(-c2ccc(NC(=O)N[C@H](C)c3ccc(C(F)F)cn3)nc2)ccc1[C@H](C)O] | LCMS-2 | 570.25 | 1.6 |
| I-299 | 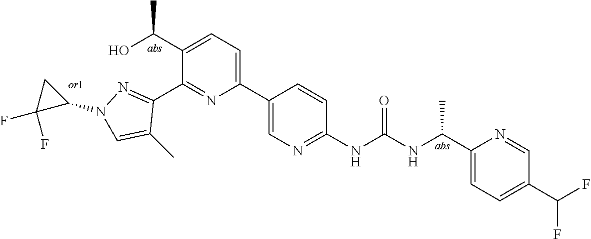 [SMILES: Cc1cn([C@H]2CC2(F)F)nc1-c1nc(-c2ccc(NC(=O)N[C@H](C)c3ccc(C(F)F)cn3)nc2)ccc1[C@H](C)O] | LCMS-2 | 570.2 | 1.59 |
| I-300 | 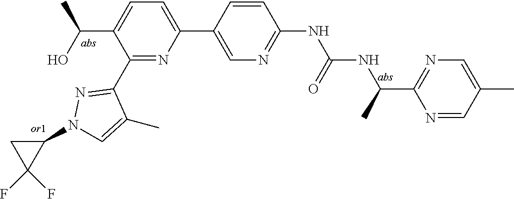 [SMILES: Cc1cnc([C@@H](C)NC(=O)Nc2ccc(-c3ccc([C@H](C)O)c(-c4nn([C@@H]5CC5(F)F)cc4C)n3)cn2)nc1] | LCMS-2 | 535.25 | 1.33 |
| I-301 | 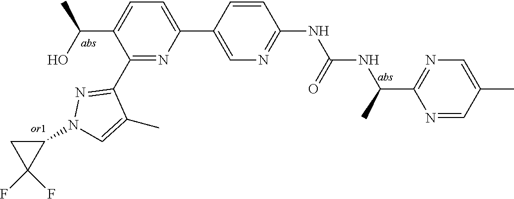 [SMILES: Cc1cnc([C@@H](C)NC(=O)Nc2ccc(-c3ccc([C@H](C)O)c(-c4nn([C@H]5CC5(F)F)cc4C)n3)cn2)nc1] | LCMS-2 | 535.25 | 1.31 |
| I-302 | 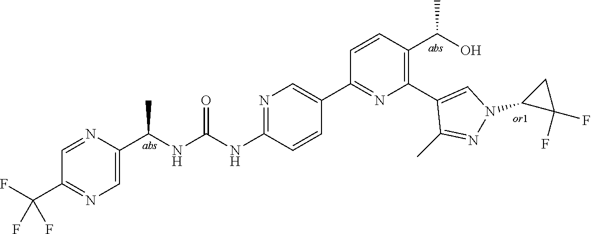 [SMILES: Cc1nn([C@@H]2CC2(F)F)cc1-c1nc(-c2ccc(NC(=O)N[C@H](C)c3cnc(C(F)(F)F)cn3)nc2)ccc1[C@H](C)O] | LCMS-2 | 589.25 | 1.48 |
| I-303 | 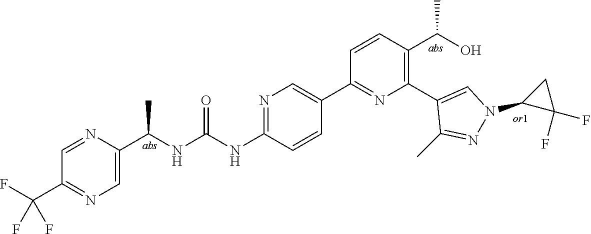 [SMILES: Cc1nn([C@H]2CC2(F)F)cc1-c1nc(-c2ccc(NC(=O)N[C@H](C)c3cnc(C(F)(F)F)cn3)nc2)ccc1[C@H](C)O] | LCMS-2 | 589.2 | 1.49 |
| I-304 | 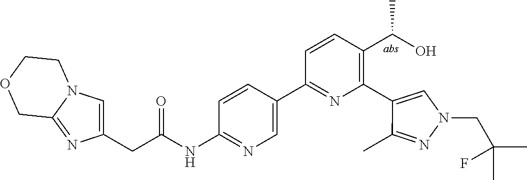 [SMILES: Cc1nn(CC(C)(C)F)cc1-c1nc(-c2ccc(NC(=O)Cc3cn4c(n3)COCC4)nc2)ccc1[C@H](C)O] | LCMS-2 | 534.25 | 0.79 |
| I-305 | 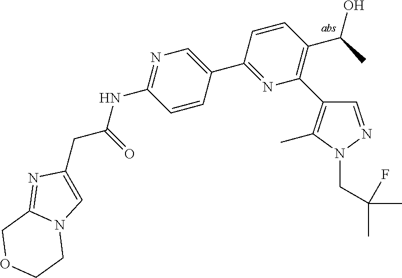 [SMILES: Cc1c(-c2nc(-c3ccc(NC(=O)Cc4cn5c(n4)COCC5)nc3)ccc2[C@H](C)O)cnn1CC(C)(C)F] | LCMS-2 | 534.2 | 0.81 |
| I-306 | 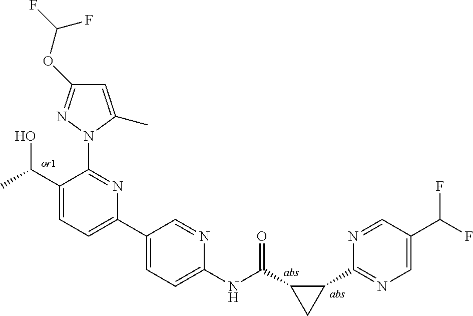 [SMILES: Cc1cc(OC(F)F)nn1-c1nc(-c2ccc(NC(=O)[C@H]3C[C@H]3c3ncc(C(F)F)cn3)nc2)ccc1[C@H](C)O] | LCMS-3 | 558.2 | 0.858 |
| I-307 | 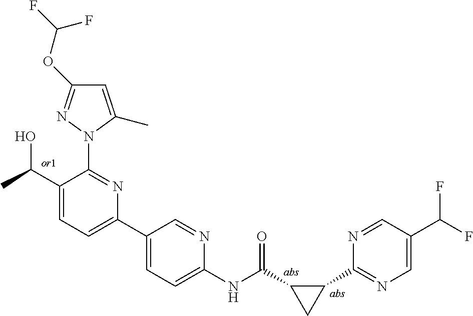 [SMILES: Cc1cc(OC(F)F)nn1-c1nc(-c2ccc(NC(=O)[C@H]3C[C@H]3c3ncc(C(F)F)cn3)nc2)ccc1[C@@H](C)O] | LCMS-3 | 558.2 | 0.86 |
| I-308 | 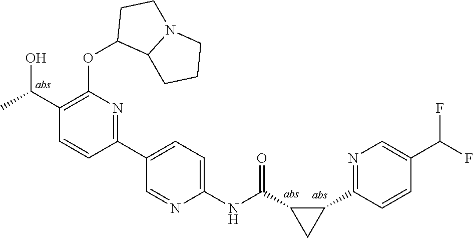 [SMILES: C[C@H](O)c1ccc(-c2ccc(NC(=O)[C@H]3C[C@H]3c3ccc(C(F)F)cn3)nc2)nc1OC1CCN2CCCC12] | LCMS-3 | 536.25 | 0.64 |
| I-309 | 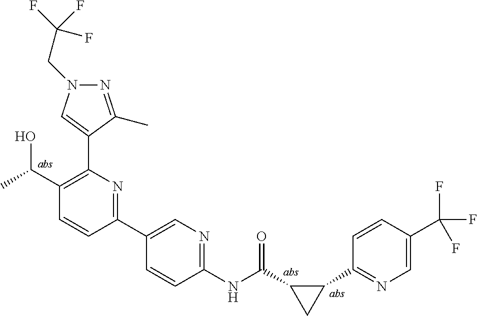 [SMILES: Cc1nn(CC(F)(F)F)cc1-c1nc(-c2ccc(NC(=O)[C@H]3C[C@H]3c3ccc(C(F)(F)F)cn3)nc2)ccc1[C@H](C)O] | LCMS-3 | 591.15 | 0.86 |
| I-310 | 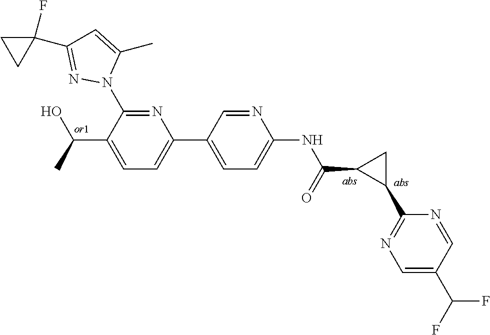 [SMILES: Cc1cc(C2(F)CC2)nn1-c1nc(-c2ccc(NC(=O)[C@H]3C[C@H]3c3ncc(C(F)F)cn3)nc2)ccc1[C@@H](C)O] | LCMS-3 | 550.2 | 0.87 |
| I-311 | 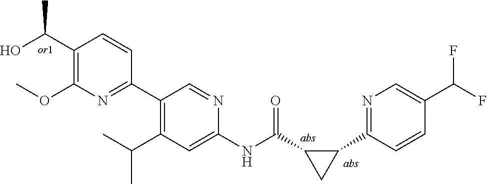 [SMILES: COc1nc(-c2cnc(NC(=O)[C@H]3C[C@H]3c3ccc(C(F)F)cn3)cc2C(C)C)ccc1[C@H](C)O] | LCMS-3 | 483.2 | 0.79 |
| I-312 | 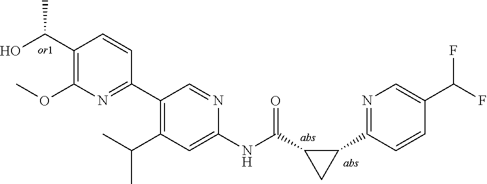 [SMILES: COc1nc(-c2cnc(NC(=O)[C@H]3C[C@H]3c3ccc(C(F)F)cn3)cc2C(C)C)ccc1[C@@H](C)O] | LCMS-3 | 483.2 | 0.79 |
| I-313 | 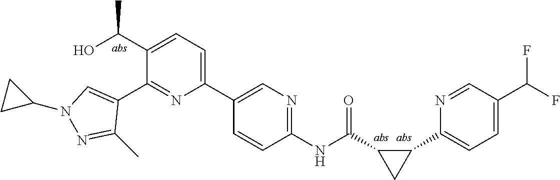 [SMILES: Cc1nn(C2CC2)cc1-c1nc(-c2ccc(NC(=O)[C@H]3C[C@H]3c3ccc(C(F)F)cn3)nc2)ccc1[C@H](C)O] | LCMS-3 | 531.25 | 0.7 |
| I-314 | 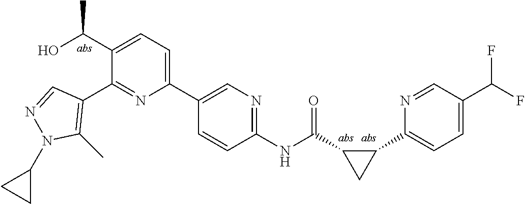 [SMILES: Cc1c(-c2nc(-c3ccc(NC(=O)[C@H]4C[C@H]4c4ccc(C(F)F)cn4)nc3)ccc2[C@H](C)O)cnn1C1CC1] | LCMS-3 | 531.25 | 0.7 |
| I-315 | 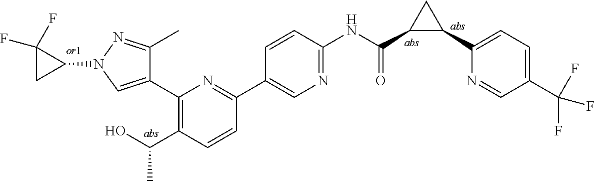 [SMILES: Cc1nn([C@@H]2CC2(F)F)cc1-c1nc(-c2ccc(NC(=O)[C@H]3C[C@H]3c3ccc(C(F)(F)F)cn3)nc2)ccc1[C@H](C)O] | LCMS-3 | 585.2 | 0.84 |
| I-316 | 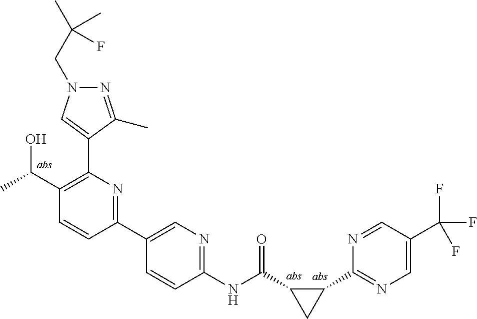 [SMILES: Cc1nn(CC(C)(C)F)cc1-c1nc(-c2ccc(NC(=O)[C@H]3C[C@H]3c3ncc(C(F)(F)F)cn3)nc2)ccc1[C@H](C)O] | LCMS-3 | 584.2 | 0.79 |
| I-317 | 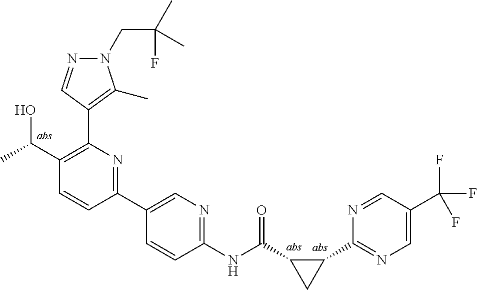 [SMILES: Cc1c(-c2nc(-c3ccc(NC(=O)[C@H]4C[C@H]4c4ncc(C(F)(F)F)cn4)nc3)ccc2[C@H](C)O)cnn1CC(C)(C)F] | LCMS-3 | 584.25 | 0.8 |
| I-318 | 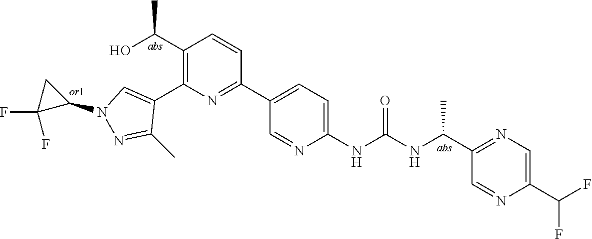 [SMILES: Cc1nn([C@@H]2CC2(F)F)cc1-c1nc(-c2ccc(NC(=O)N[C@H](C)c3cnc(C(F)F)cn3)nc2)ccc1[C@H](C)O] | LCMS-3 | 571.2 | 0.77 |
| I-319 | 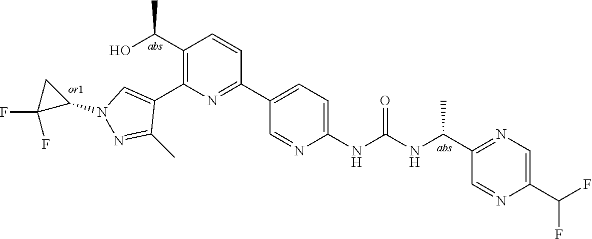 [SMILES: Cc1nn([C@H]2CC2(F)F)cc1-c1nc(-c2ccc(NC(=O)N[C@H](C)c3cnc(C(F)F)cn3)nc2)ccc1[C@H](C)O] | LCMS-3 | 571.2 | 0.78 |
| I-320 | 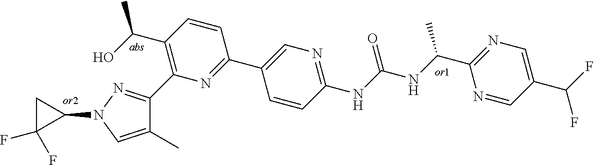 [SMILES: Cc1cn([C@@H]2CC2(F)F)nc1-c1nc(-c2ccc(NC(=O)N[C@H](C)c3ncc(C(F)F)cn3)nc2)ccc1[C@H](C)O] | LCMS-3 | 571.25 | 0.84 |
| I-321 | 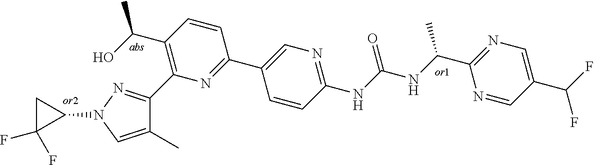 [SMILES: Cc1cn([C@H]2CC2(F)F)nc1-c1nc(-c2ccc(NC(=O)N[C@H](C)c3ncc(C(F)F)cn3)nc2)ccc1[C@H](C)O] | LCMS-3 | 571.25 | 0.84 |
| I-322 | 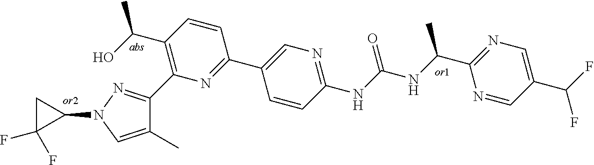 [SMILES: Cc1cn([C@@H]2CC2(F)F)nc1-c1nc(-c2ccc(NC(=O)N[C@@H](C)c3ncc(C(F)F)cn3)nc2)ccc1[C@H](C)O] | LCMS-3 | 571.2 | 0.83 |
| I-323 | 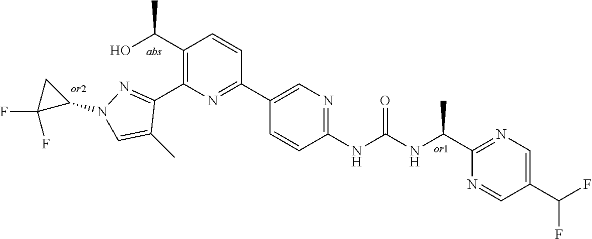 [SMILES: Cc1cn([C@H]2CC2(F)F)nc1-c1nc(-c2ccc(NC(=O)N[C@@H](C)c3ncc(C(F)F)cn3)nc2)ccc1[C@H](C)O] | LCMS-3 | 571.25 | 0.84 |
| I-324 | 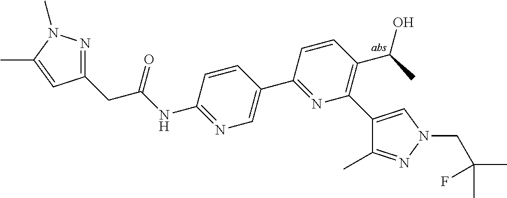 [SMILES: Cc1nn(CC(C)(C)F)cc1-c1nc(-c2ccc(NC(=O)Cc3cc(C)n(C)n3)nc2)ccc1[C@H](C)O] | LCMS-3 | 506.25 | 0.72 |
| I-325 | 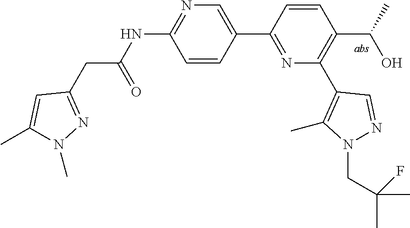 [SMILES: Cc1cc(CC(=O)Nc2ccc(-c3ccc([C@H](C)O)c(-c4cnn(CC(C)(C)F)c4C)n3)cn2)nn1C] | LCMS-3 | 506.25 | 0.72 |
| I-326 | 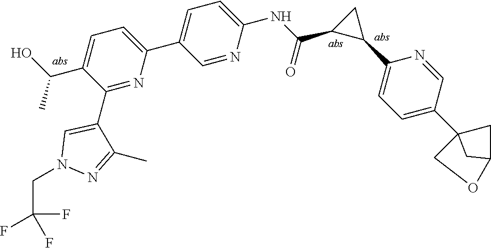 [SMILES: Cc1nn(CC(F)(F)F)cc1-c1nc(-c2ccc(NC(=O)[C@H]3C[C@H]3c3ccc(C45COC(C4)C5)cn3)nc2)ccc1[C@H](C)O] | LCMS-4 | 605.1 | 0.416 |
| I-327 | 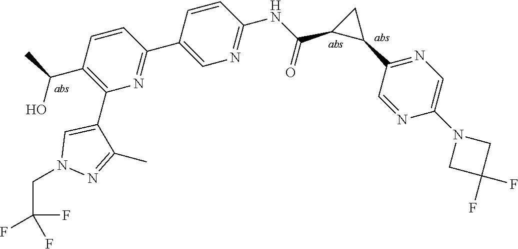 [SMILES: Cc1nn(CC(F)(F)F)cc1-c1nc(-c2ccc(NC(=O)[C@H]3C[C@H]3c3cnc(N4CC(F)(F)C4)cn3)nc2)ccc1[C@H](C)O] | LCMS-4 | 615.3 | 0.477 |
| I-328 | 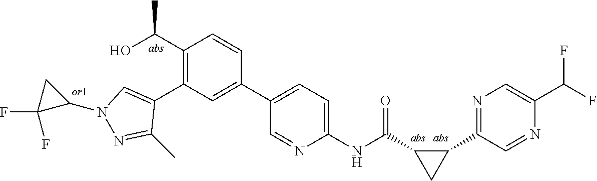 [SMILES: Cc1nn(C2CC2(F)F)cc1-c1cc(-c2ccc(NC(=O)[C@H]3C[C@H]3c3cnc(C(F)F)cn3)nc2)ccc1[C@H](C)O] | LCMS-4 | 568.2 | 0.463 |
| I-329 | 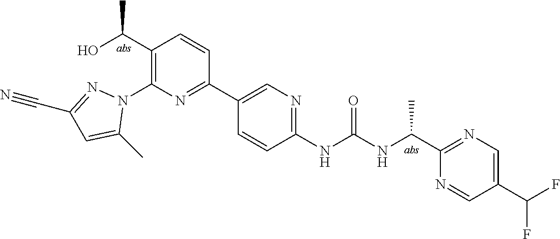 [SMILES: Cc1cc(C#N)nn1-c1nc(-c2ccc(NC(=O)N[C@H](C)c3ncc(C(F)F)cn3)nc2)ccc1[C@H](C)O] | LCMS-4 | 520.3 | 0.487 |
| I-330 | 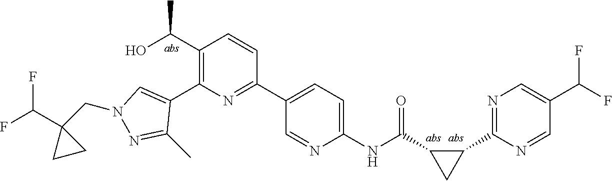 [SMILES: Cc1nn(CC2(C(F)F)CC2)cc1-c1nc(-c2ccc(NC(=O)[C@H]3C[C@H]3c3ncc(C(F)F)cn3)nc2)ccc1[C@H](C)O] | LCMS-4 | 596.1 | 0.469 |
| I-331 | 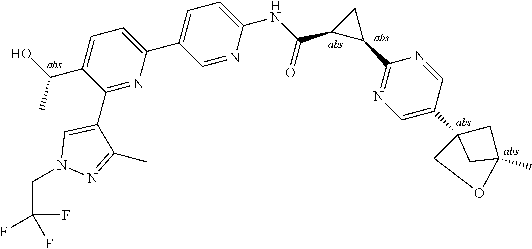 [SMILES: Cc1nn(CC(F)(F)F)cc1-c1nc(-c2ccc(NC(=O)[C@H]3C[C@H]3c3ncc([C@]45CO[C@](C)(C4)C5)cn3)nc2)ccc1[C@H](C)O] | LCMS-4 | 620.3 | 0.447 |
| I-332 | 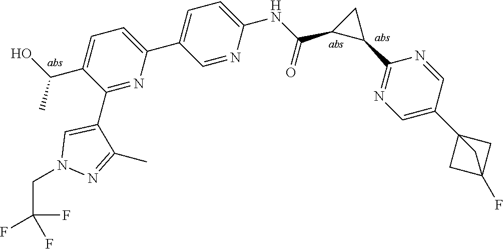 [SMILES: Cc1nn(CC(F)(F)F)cc1-c1nc(-c2ccc(NC(=O)[C@H]3C[C@H]3c3ncc(C45CC(F)(C4)C5)cn3)nc2)ccc1[C@H](C)O] | LCMS-4 | 608.1 | 0.484 |
| I-333 | 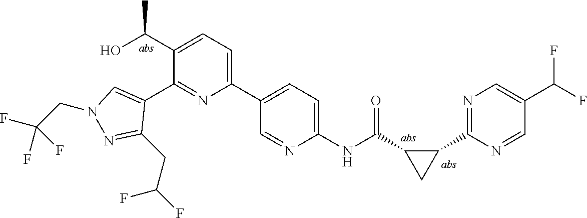 [SMILES: C[C@H](O)c1ccc(-c2ccc(NC(=O)[C@H]3C[C@H]3c3ncc(C(F)F)cn3)nc2)nc1-c1cn(CC(F)(F)F)nc1CC(F)F] | LCMS-4 | 624.2 | 0.485 |
| I-334 | 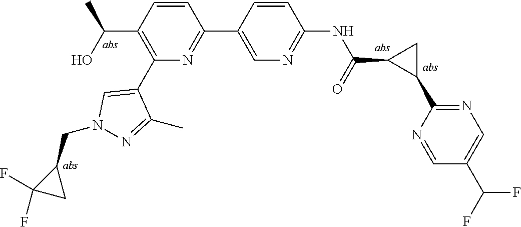 [SMILES: Cc1nn(C[C@H]2CC2(F)F)cc1-c1nc(-c2ccc(NC(=O)[C@H]3C[C@H]3c3ncc(C(F)F)cn3)nc2)ccc1[C@H](C)O] | LCMS-4 | 582.1 | 0.458 |
| I-335 | 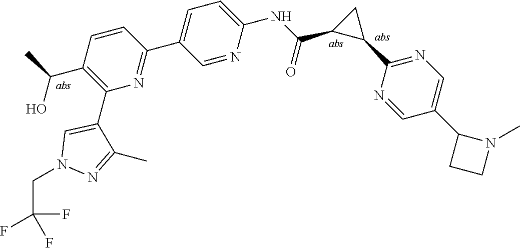 [SMILES: Cc1nn(CC(F)(F)F)cc1-c1nc(-c2ccc(NC(=O)[C@H]3C[C@H]3c3ncc(C4CCN4C)cn3)nc2)ccc1[C@H](C)O] | LCMS-4 | 593.3 | 0.403 |
| I-336 | 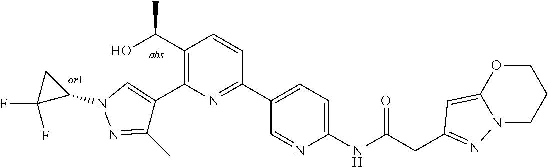 [SMILES: Cc1nn([C@H]2CC2(F)F)cc1-c1nc(-c2ccc(NC(=O)Cc3cc4n(n3)CCCO4)nc2)ccc1[C@H](C)O] | LCMS-4 | 536.3 | 0.423 |
| I-337 | 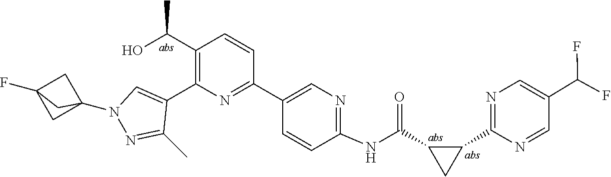 [SMILES: Cc1nn(C23CC(F)(C2)C3)cc1-c1nc(-c2ccc(NC(=O)[C@H]3C[C@H]3c3ncc(C(F)F)cn3)nc2)ccc1[C@H](C)O] | LCMS-4 | 576.4 | 0.472 |
| I-338 | 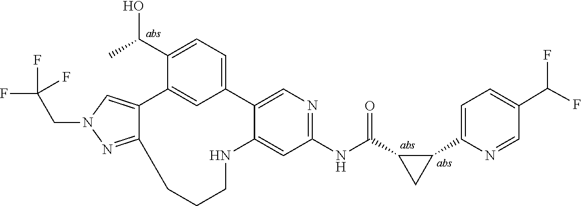 [SMILES: C[C@H](O)c1ccc2cc1-c1cn(CC(F)(F)F)nc1CCCNc1cc(NC(=O)[C@H]3C[C@H]3c3ccc(C(F)F)cn3)ncc1-2] | LCMS-4 | 614.3 | 0.467 |
| I-339 | 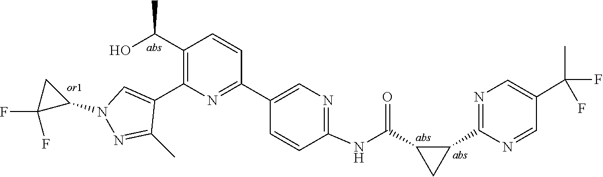 [SMILES: Cc1nn([C@H]2CC2(F)F)cc1-c1nc(-c2ccc(NC(=O)[C@H]3C[C@H]3c3ncc(C(C)(F)F)cn3)nc2)ccc1[C@H](C)O] | LCMS-4 | 582.3 | 0.466 |
| I-340 | 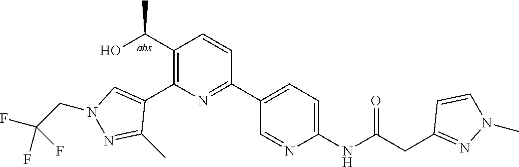 [SMILES: Cc1nn(CC(F)(F)F)cc1-c1nc(-c2ccc(NC(=O)Cc3ccn(C)n3)nc2)ccc1[C@H](C)O] | LCMS-4 | 500 | 0.443 |
| I-341 | 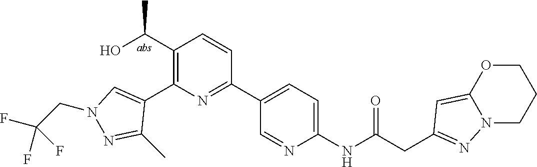 [SMILES: Cc1nn(CC(F)(F)F)cc1-c1nc(-c2ccc(NC(=O)Cc3cc4n(n3)CCCO4)nc2)ccc1[C@H](C)O] | LCMS-4 | 542.3 | 0.449 |
| I-342 | 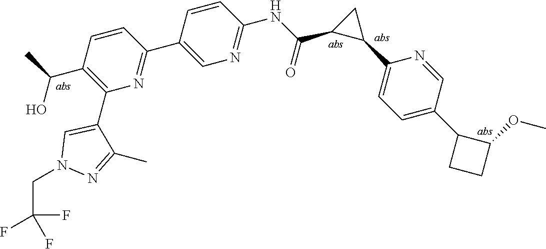 [SMILES: CO[C@@H]1CCC1c1ccc([C@@H]2C[C@@H]2C(=O)Nc2ccc(-c3ccc([C@H](C)O)c(-c4cn(CC(F)(F)F)nc4C)n3)cn2)nc1] | LCMS-4 | 607.4 | 0.455 |
| I-343 | 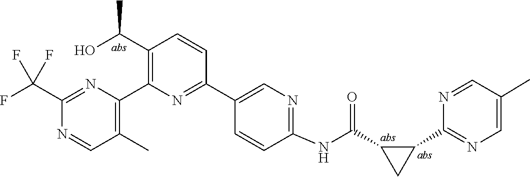 [SMILES: Cc1cnc([C@@H]2C[C@@H]2C(=O)Nc2ccc(-c3ccc([C@H](C)O)c(-c4nc(C(F)(F)F)ncc4C)n3)cn2)nc1] | LCMS-4 | 536.1 | 0.477 |
| I-344 | 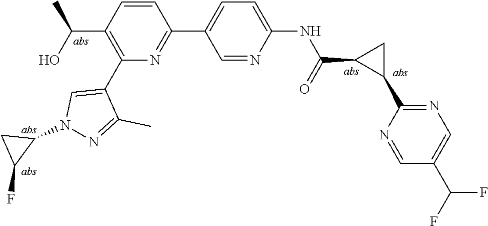 [SMILES: Cc1nn([C@H]2C[C@@H]2F)cc1-c1nc(-c2ccc(NC(=O)[C@H]3C[C@H]3c3ncc(C(F)F)cn3)nc2)ccc1[C@H](C)O] | LCMS-4 | 550.2 | 0.438 |
| I-345 | 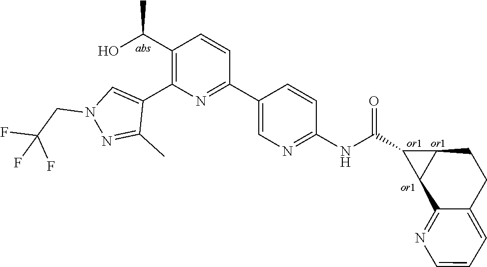 [SMILES: Cc1nn(CC(F)(F)F)cc1-c1nc(-c2ccc(NC(=O)[C@@H]3[C@H]4CCc5cccnc5[C@H]43)nc2)ccc1[C@H](C)O] | LCMS-4 | 549.3 | 0.424 |
| I-346 | 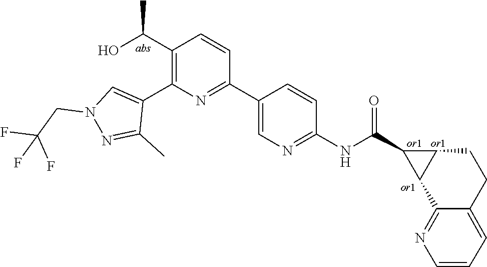 [SMILES: Cc1nn(CC(F)(F)F)cc1-c1nc(-c2ccc(NC(=O)[C@H]3[C@@H]4CCc5cccnc5[C@@H]43)nc2)ccc1[C@H](C)O] | LCMS-4 | 549.2 | 0.425 |
| I-347 | 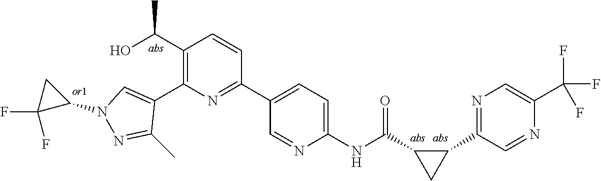 [SMILES: Cc1nn([C@H]2CC2(F)F)cc1-c1nc(-c2ccc(NC(=O)[C@H]3C[C@H]3c3cnc(C(F)(F)F)cn3)nc2)ccc1[C@H](C)O] | LCMS-4 | 586.2 | 0.497 |
| I-348 | 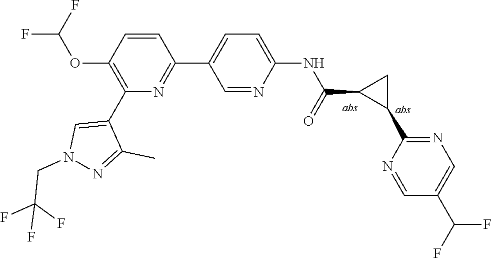 [SMILES: Cc1nn(CC(F)(F)F)cc1-c1nc(-c2ccc(NC(=O)[C@H]3C[C@H]3c3ncc(C(F)F)cn3)nc2)ccc1OC(F)F] | LCMS-4 | 596 | 0.532 |
| I-349 | 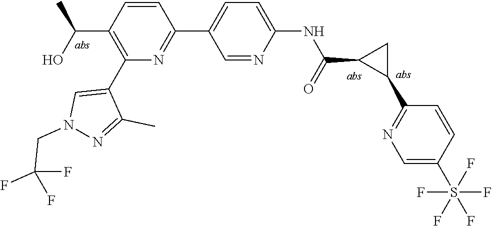 [SMILES: Cc1nn(CC(F)(F)F)cc1-c1nc(-c2ccc(NC(=O)[C@H]3C[C@H]3c3ccc(S(F)(F)(F)(F)F)cn3)nc2)ccc1[C@H](C)O] | LCMS-4 | 649.1 | 0.521 |
| I-350 | 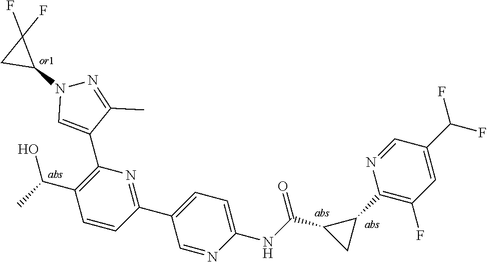 [SMILES: Cc1nn([C@H]2CC2(F)F)cc1-c1nc(-c2ccc(NC(=O)[C@H]3C[C@H]3c3ncc(C(F)F)cc3F)nc2)ccc1[C@H](C)O] | LCMS-4 | 585.3 | 0.477 |
| I-351 | 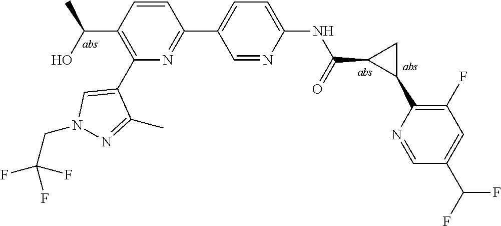 [SMILES: Cc1nn(CC(F)(F)F)cc1-c1nc(-c2ccc(NC(=O)[C@H]3C[C@H]3c3ncc(C(F)F)cc3F)nc2)ccc1[C@H](C)O] | LCMS-4 | 591.2 | 0.488 |
| I-352 | 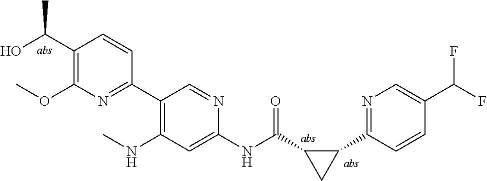 [SMILES: CNc1cc(NC(=O)[C@H]2C[C@H]2c2ccc(C(F)F)cn2)ncc1-c1ccc([C@H](C)O)c(OC)n1] | LCMS-4 | 470.1 | 0.476 |
| I-353 | 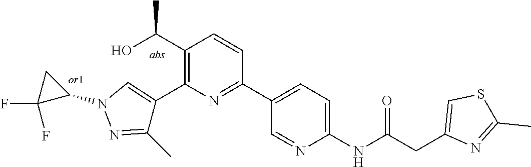 [SMILES: Cc1nc(CC(=O)Nc2ccc(-c3ccc([C@H](C)O)c(-c4cn([C@H]5CC5(F)F)nc4C)n3)cn2)cs1] | LCMS-4 | 511.3 | 0.451 |
| I-354 | 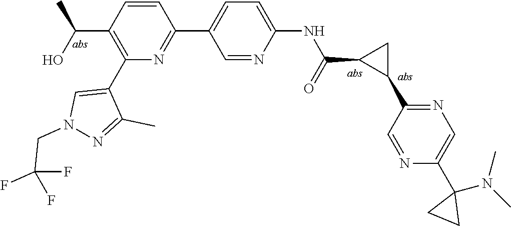 [SMILES: Cc1nn(CC(F)(F)F)cc1-c1nc(-c2ccc(NC(=O)[C@H]3C[C@H]3c3cnc(C4(N(C)C)CC4)cn3)nc2)ccc1[C@H](C)O] | LCMS-4 | 607.3 | 0.404 |
| I-355 | 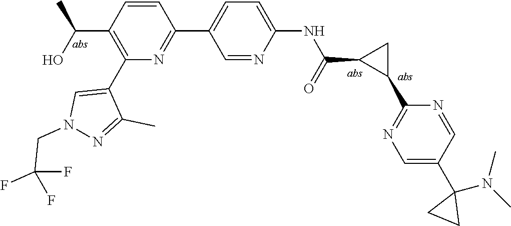 [SMILES: Cc1nn(CC(F)(F)F)cc1-c1nc(-c2ccc(NC(=O)[C@H]3C[C@H]3c3ncc(C4(N(C)C)CC4)cn3)nc2)ccc1[C@H](C)O] | LCMS-4 | 607.3 | 0.402 |
| I-356 | 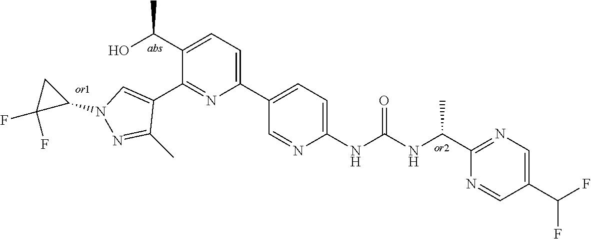 [SMILES: Cc1nn([C@H]2CC2(F)F)cc1-c1nc(-c2ccc(NC(=O)N[C@H](C)c3ncc(C(F)F)cn3)nc2)ccc1[C@H](C)O] | LCMS-4 | 572.2 | 0.48 |
| I-357 | 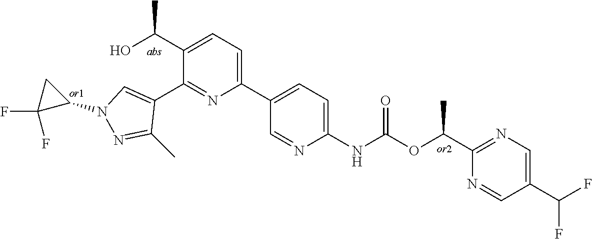 [SMILES: Cc1nn([C@H]2CC2(F)F)cc1-c1nc(-c2ccc(NC(=O)O[C@@H](C)c3ncc(C(F)F)cn3)nc2)ccc1[C@H](C)O] | LCMS-4 | 572.3 | 0.487 |
| I-358 | 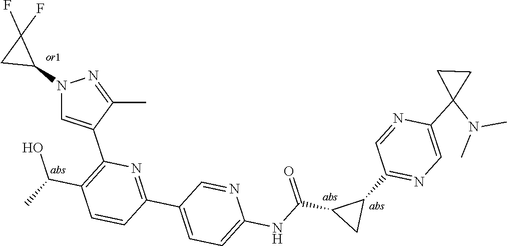 [SMILES: Cc1nn([C@H]2CC2(F)F)cc1-c1nc(-c2ccc(NC(=O)[C@H]3C[C@H]3c3cnc(C4(N(C)C)CC4)cn3)nc2)ccc1[C@H](C)O] | LCMS-4 | 601.4 | 0.405 |
| I-359 | 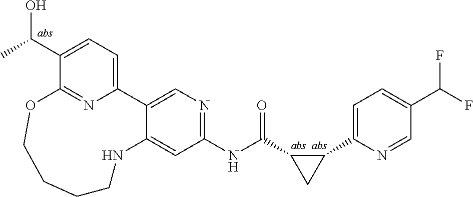 [SMILES: C[C@H](O)c1ccc2nc1OCCCCNc1cc(NC(=O)[C@H]3C[C@H]3c3ccc(C(F)F)cn3)ncc1-2] | LCMS-4 | 496.3 | 0.454 |
| I-360 | 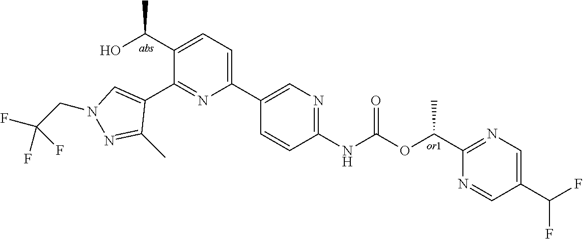 [SMILES: Cc1nn(CC(F)(F)F)cc1-c1nc(-c2ccc(NC(=O)O[C@H](C)c3ncc(C(F)F)cn3)nc2)ccc1[C@H](C)O] | LCMS-4 | 578.3 | 0.488 |
| I-361 | 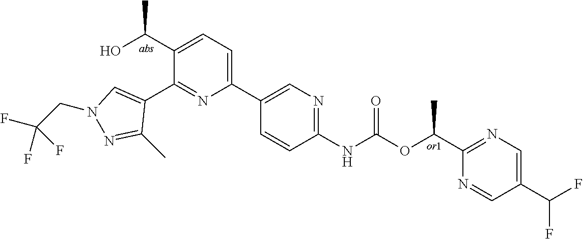 [SMILES: Cc1nn(CC(F)(F)F)cc1-c1nc(-c2ccc(NC(=O)O[C@@H](C)c3ncc(C(F)F)cn3)nc2)ccc1[C@H](C)O] | LCMS-4 | 578.3 | 0.483 |
| I-362 | 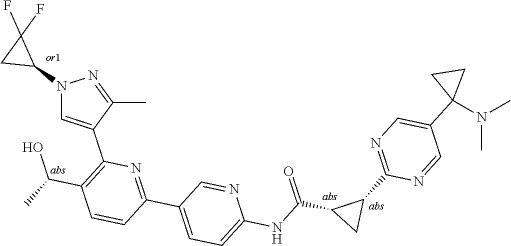 [SMILES: Cc1nn([C@H]2CC2(F)F)cc1-c1nc(-c2ccc(NC(=O)[C@H]3C[C@H]3c3ncc(C4(N(C)C)CC4)cn3)nc2)ccc1[C@H](C)O] | LCMS-4 | 601.5 | 0.391 |
| I-363 | 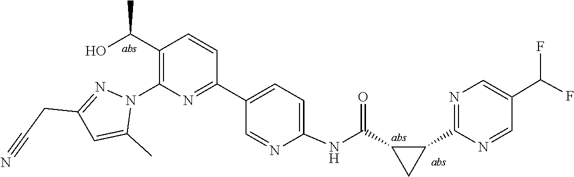 [SMILES: Cc1cc(CC#N)nn1-c1nc(-c2ccc(NC(=O)[C@H]3C[C@H]3c3ncc(C(F)F)cn3)nc2)ccc1[C@H](C)O] | LCMS-4 | 531.2 | 0.456 |
| I-364 | 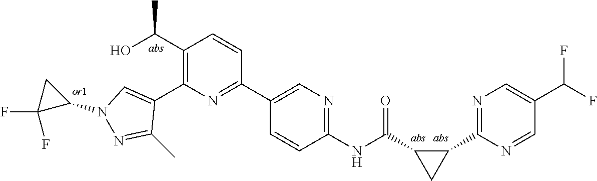 [SMILES: Cc1nn([C@H]2CC2(F)F)cc1-c1nc(-c2ccc(NC(=O)[C@H]3C[C@H]3c3ncc(C(F)F)cn3)nc2)ccc1[C@H](C)O] | LCMS-4 | 569.3 | 0.496 |
| I-365 | 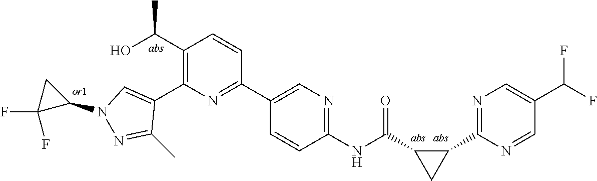 [SMILES: Cc1nn([C@@H]2CC2(F)F)cc1-c1nc(-c2ccc(NC(=O)[C@H]3C[C@H]3c3ncc(C(F)F)cn3)nc2)ccc1[C@H](C)O] | LCMS-4 | 569.4 | 0.492 |
| I-366 | 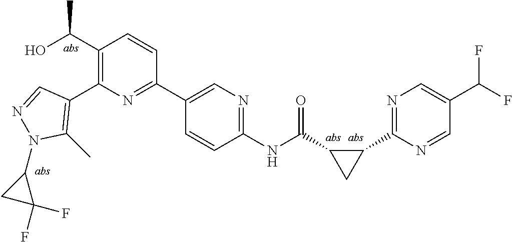 [SMILES: Cc1c(-c2nc(-c3ccc(NC(=O)[C@H]4C[C@H]4c4ncc(C(F)F)cn4)nc3)ccc2[C@H](C)O)cnn1C1CC1(F)F] | LCMS-4 | 569.3 | 0.469 |
| I-367 | 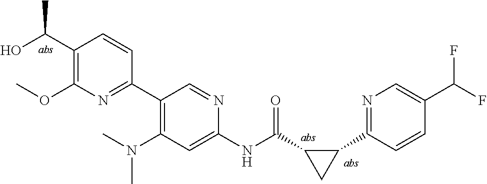 [SMILES: COc1nc(-c2cnc(NC(=O)[C@H]3C[C@H]3c3ccc(C(F)F)cn3)cc2N(C)C)ccc1[C@H](C)O] | LCMS-4 | 484.3 | 0.438 |
| I-368 | 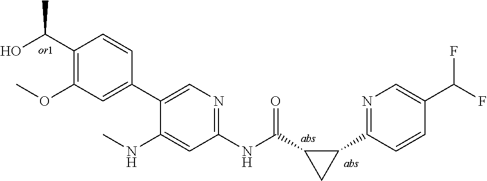 [SMILES: CNc1cc(NC(=O)[C@H]2C[C@H]2c2ccc(C(F)F)cn2)ncc1-c1ccc([C@H](C)O)c(OC)c1] | LCMS-4 | 470.2 | 0.437 |
| I-369 | 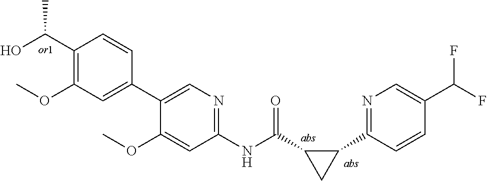 [SMILES: COc1cc(NC(=O)[C@H]2C[C@H]2c2ccc(C(F)F)cn2)ncc1-c1ccc([C@@H](C)O)c(OC)c1] | LCMS-4 | 470.2 | 0.443 |
| I-370 | 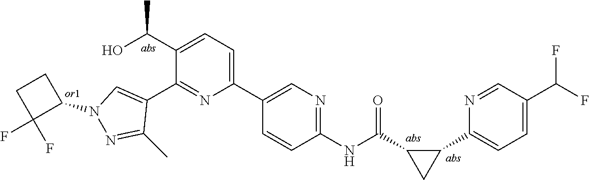 [SMILES: Cc1nn([C@H]2CCC2(F)F)cc1-c1nc(-c2ccc(NC(=O)[C@H]3C[C@H]3c3ccc(C(F)F)cn3)nc2)ccc1[C@H](C)O] | LCMS-4 | 581.3 | 0.465 |
| I-371 | 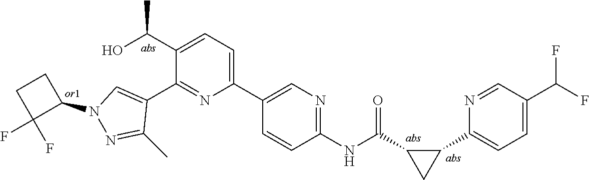 [SMILES: Cc1nn([C@@H]2CCC2(F)F)cc1-c1nc(-c2ccc(NC(=O)[C@H]3C[C@H]3c3ccc(C(F)F)cn3)nc2)ccc1[C@H](C)O] | LCMS-4 | 581.5 | 0.462 |
| I-372 | 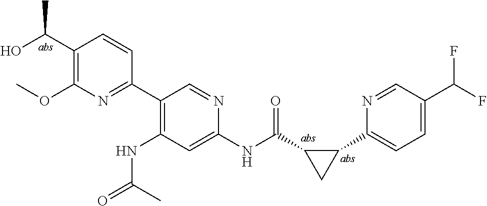 [SMILES: COc1nc(-c2cnc(NC(=O)[C@H]3C[C@H]3c3ccc(C(F)F)cn3)cc2NC(C)=O)ccc1[C@H](C)O] | LCMS-4 | 498.3 | 0.444 |
| I-373 | 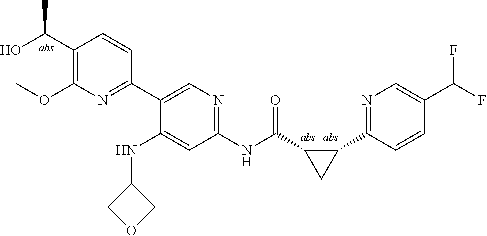 [SMILES: COc1nc(-c2cnc(NC(=O)[C@H]3C[C@H]3c3ccc(C(F)F)cn3)cc2NC2COC2)ccc1[C@H](C)O] | LCMS-4 | 512.2 | 0.43 |
| I-374 | 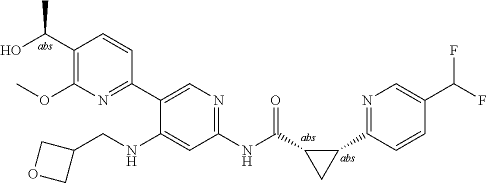 [SMILES: COc1nc(-c2cnc(NC(=O)[C@H]3C[C@H]3c3ccc(C(F)F)cn3)cc2NCC2COC2)ccc1[C@H](C)O] | LCMS-4 | 526.2 | 0.432 |
| I-375 | 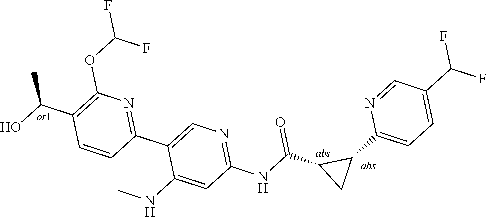 [SMILES: CNc1cc(NC(=O)[C@H]2C[C@H]2c2ccc(C(F)F)cn2)ncc1-c1ccc([C@H](C)O)c(OC(F)F)n1] | LCMS-4 | 506.1 | 0.449 |
| I-376 | 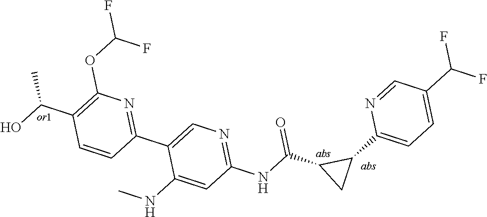 [SMILES: CNc1cc(NC(=O)[C@H]2C[C@H]2c2ccc(C(F)F)cn2)ncc1-c1ccc([C@@H](C)O)c(OC(F)F)n1] | LCMS-4 | 506.2 | 0.439 |
| I-377 | 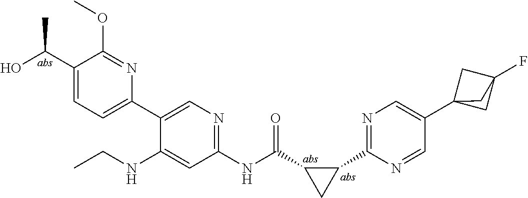 [SMILES: CCNc1cc(NC(=O)[C@H]2C[C@H]2c2ncc(C34CC(F)(C3)C4)cn2)ncc1-c1ccc([C@H](C)O)c(OC)n1] | LCMS-4 | 519.3 | 0.465 |
| I-378 | 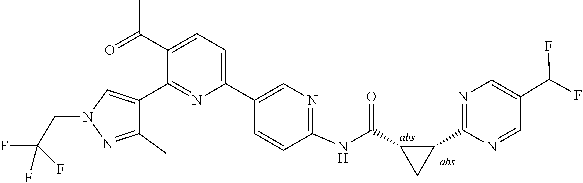 [SMILES: CC(=O)c1ccc(-c2ccc(NC(=O)[C@H]3C[C@H]3c3ncc(C(F)F)cn3)nc2)nc1-c1cn(CC(F)(F)F)nc1C] | LCMS-4 | 572.3 | 0.49 |
| I-379 | 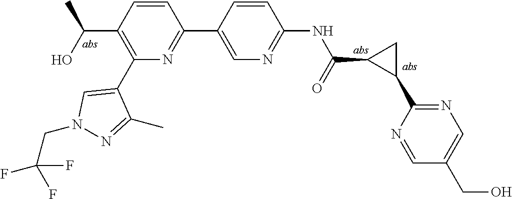 [SMILES: Cc1nn(CC(F)(F)F)cc1-c1nc(-c2ccc(NC(=O)[C@H]3C[C@H]3c3ncc(CO)cn3)nc2)ccc1[C@H](C)O] | LCMS-4 | 554.2 | 0.406 |
| I-380 | 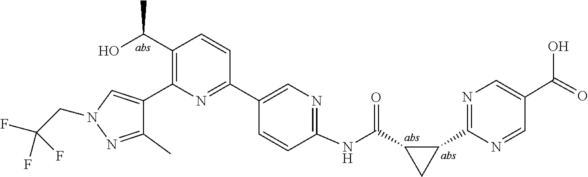 [SMILES: Cc1nn(CC(F)(F)F)cc1-c1nc(-c2ccc(NC(=O)[C@H]3C[C@H]3c3ncc(C(=O)O)cn3)nc2)ccc1[C@H](C)O] | LCMS-4 | 568.4 | 0.412 |
| I-381 | 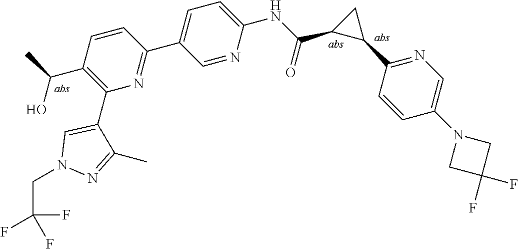 [SMILES: Cc1nn(CC(F)(F)F)cc1-c1nc(-c2ccc(NC(=O)[C@H]3C[C@H]3c3ccc(N4CC(F)(F)C4)cn3)nc2)ccc1[C@H](C)O] | LCMS-5 | 614.3 | 0.607 |
| I-382 | 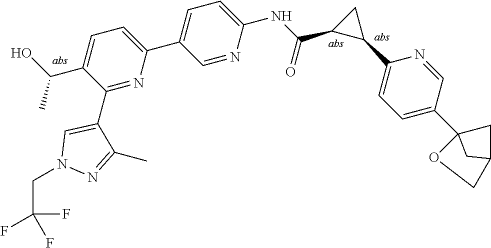 [SMILES: Cc1nn(CC(F)(F)F)cc1-c1nc(-c2ccc(NC(=O)[C@H]3C[C@H]3c3ccc(C45CC(CO4)C5)cn3)nc2)ccc1[C@H](C)O] | LCMS-5 | 605.3 | 0.599 |
| I-383 | 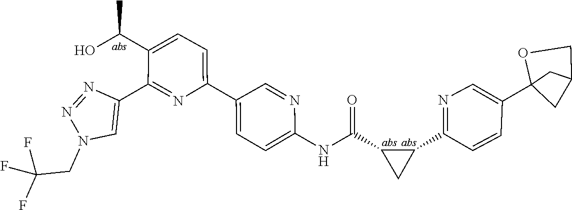 | LCMS-5 | 606.3 | 0.614 |
| I-384 | 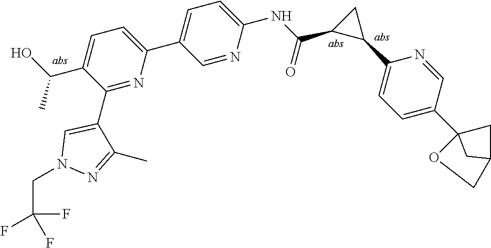 [SMILES: Cc1nn(CC(F)(F)F)cc1-c1nc(-c2ccc(NC(=O)[C@H]3C[C@H]3c3ccc(C45CC(CO4)C5)cn3)nc2)ccc1[C@H](C)O] | LCMS-5 | 606.3 | 0.556 |
| I-385 | 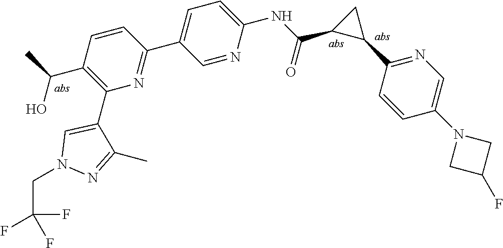 [SMILES: Cc1nn(CC(F)(F)F)cc1-c1nc(-c2ccc(NC(=O)[C@H]3C[C@H]3c3ccc(N4CC(F)C4)cn3)nc2)ccc1[C@H](C)O] | LCMS-5 | 596.3 | 0.596 |
| I-386 | 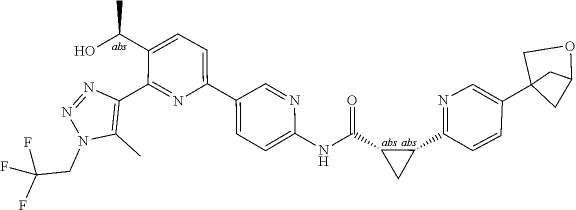 [SMILES: Cc1c(-c2nc(-c3ccc(NC(=O)[C@H]4C[C@H]4c4ccc(C56COC(C5)C6)cn4)nc3)ccc2[C@H](C)O)nnn1CC(F)(F)F] | LCMS-5 | 606.3 | 0.592 |
| I-387 | 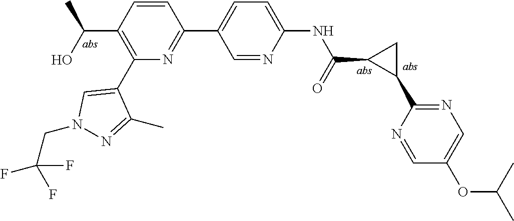 [SMILES: Cc1nn(CC(F)(F)F)cc1-c1nc(-c2ccc(NC(=O)[C@H]3C[C@H]3c3ncc(OC(C)C)cn3)nc2)ccc1[C@H](C)O] | LCMS-5 | 582.3 | 0.591 |
| I-388 | 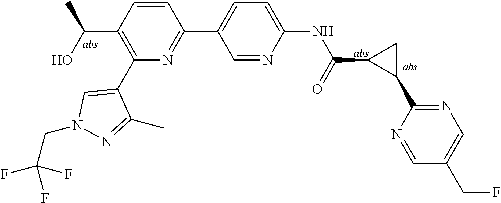 [SMILES: Cc1nn(CC(F)(F)F)cc1-c1nc(-c2ccc(NC(=O)[C@H]3C[C@H]3c3ncc(CF)cn3)nc2)ccc1[C@H](C)O] | LCMS-5 | 556.3 | 0.534 |
| I-389 | 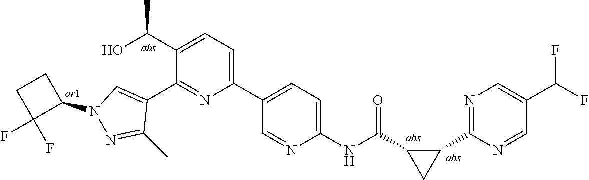 [SMILES: Cc1nn([C@@H]2CCC2(F)F)cc1-c1nc(-c2ccc(NC(=O)[C@H]3C[C@H]3c3ncc(C(F)F)cn3)nc2)ccc1[C@H](C)O] | LCMS-5 | 582.3 | 0.563 |
| I-390 | 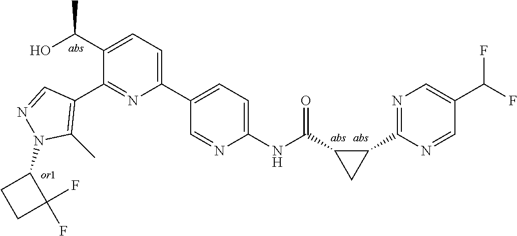 [SMILES: Cc1c(-c2nc(-c3ccc(NC(=O)[C@H]4C[C@H]4c4ncc(C(F)F)cn4)nc3)ccc2[C@H](C)O)cnn1[C@H]1CCC1(F)F] | LCMS-5 | 582.3 | 0.573 |
| I-391 | 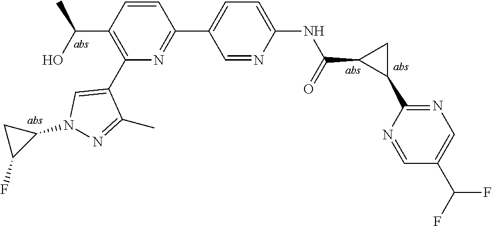 [SMILES: Cc1nn([C@H]2C[C@H]2F)cc1-c1nc(-c2ccc(NC(=O)[C@H]3C[C@H]3c3ncc(C(F)F)cn3)nc2)ccc1[C@H](C)O] | LCMS-5 | 550.3 | 0.515 |
| I-392 | 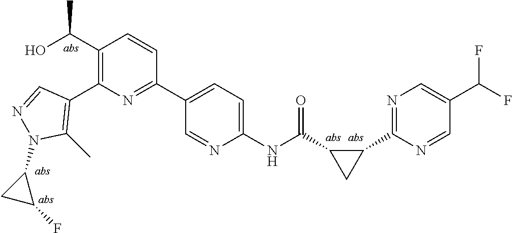 [SMILES: Cc1c(-c2nc(-c3ccc(NC(=O)[C@H]4C[C@H]4c4ncc(C(F)F)cn4)nc3)ccc2[C@H](C)O)cnn1[C@H]1C[C@H]1F] | LCMS-5 | 550.3 | 0.53 |
| I-393 | 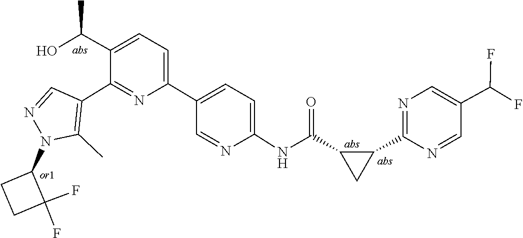 [SMILES: Cc1c(-c2nc(-c3ccc(NC(=O)[C@H]4C[C@H]4c4ncc(C(F)F)cn4)nc3)ccc2[C@H](C)O)cnn1[C@@H]1CCC1(F)F] | LCMS-5 | 582.3 | 0.575 |
| I-394 | 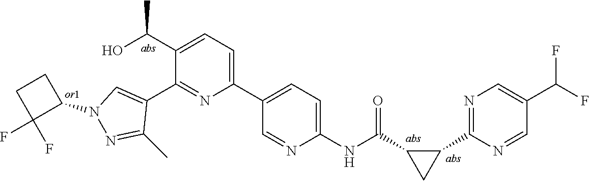 [SMILES: Cc1nn([C@H]2CCC2(F)F)cc1-c1nc(-c2ccc(NC(=O)[C@H]3C[C@H]3c3ncc(C(F)F)cn3)nc2)ccc1[C@H](C)O] | LCMS-5 | 582.2 | 0.559 |
| I-395 | 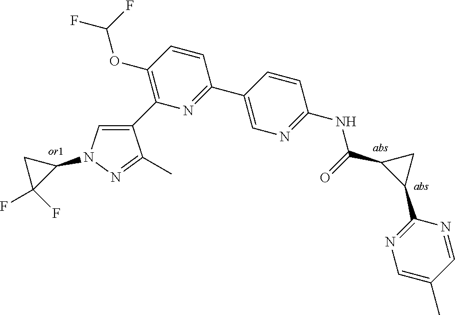 [SMILES: Cc1cnc([C@@H]2C[C@@H]2C(=O)Nc2ccc(-c3ccc(OC(F)F)c(-c4cn([C@@H]5CC5(F)F)nc4C)n3)cn2)nc1] | LCMS-5 | 554.2 | 0.634 |
| I-396 | 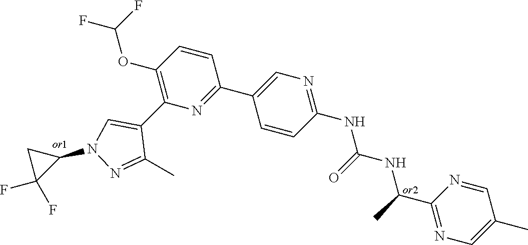 [SMILES: Cc1cnc([C@@H](C)NC(=O)Nc2ccc(-c3ccc(OC(F)F)c(-c4cn([C@@H]5CC5(F)F)nc4C)n3)cn2)nc1] | LCMS-5 | 557.3 | 0.659 |
| I-397 | 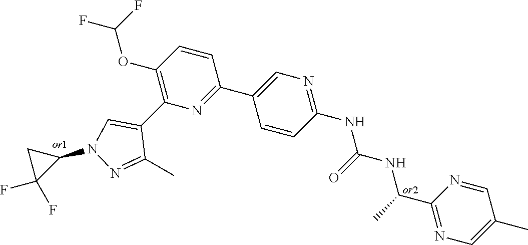 [SMILES: Cc1cnc([C@H](C)NC(=O)Nc2ccc(-c3ccc(OC(F)F)c(-c4cn([C@@H]5CC5(F)F)nc4C)n3)cn2)nc1] | LCMS-5 | 557.3 | 0.665 |
| I-398 | 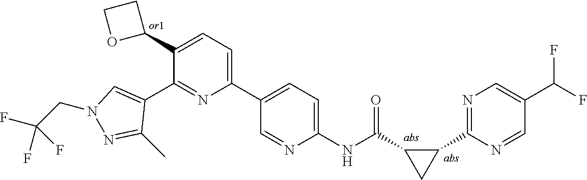 [SMILES: Cc1nn(CC(F)(F)F)cc1-c1nc(-c2ccc(NC(=O)[C@H]3C[C@H]3c3ncc(C(F)F)cn3)nc2)ccc1[C@@H]1CCO1] | LCMS-5 | 586.3 | 0.618 |
| I-399 | 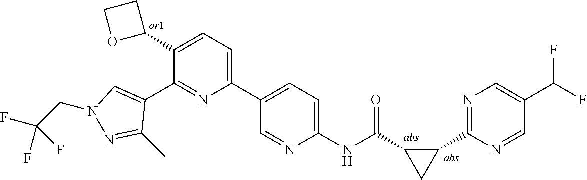 [SMILES: Cc1nn(CC(F)(F)F)cc1-c1nc(-c2ccc(NC(=O)[C@H]3C[C@H]3c3ncc(C(F)F)cn3)nc2)ccc1[C@H]1CCO1] | LCMS-5 | 586.3 | 0.613 |
| I-400 | 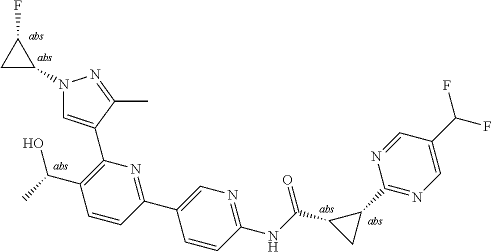 [SMILES: Cc1nn([C@@H]2C[C@@H]2F)cc1-c1nc(-c2ccc(NC(=O)[C@H]3C[C@H]3c3ncc(C(F)F)cn3)nc2)ccc1[C@H](C)O] | LCMS-5 | 550.3 | 0.52 |
| I-401 | 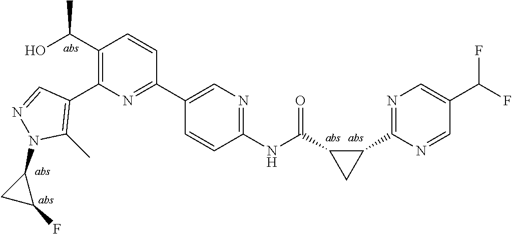 [SMILES: Cc1c(-c2nc(-c3ccc(NC(=O)[C@H]4C[C@H]4c4ncc(C(F)F)cn4)nc3)ccc2[C@H](C)O)cnn1[C@@H]1C[C@@H]1F] | LCMS-5 | 550.3 | 0.522 |
| I-402 | 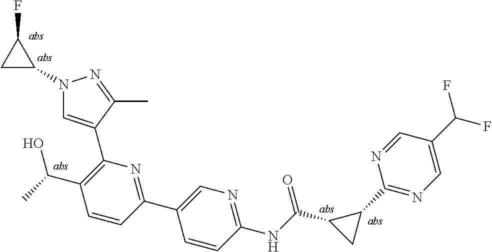 [SMILES: Cc1nn([C@@H]2C[C@H]2F)cc1-c1nc(-c2ccc(NC(=O)[C@H]3C[C@H]3c3ncc(C(F)F)cn3)nc2)ccc1[C@H](C)O] | LCMS-5 | 550.3 | 0.538 |
| I-403 | 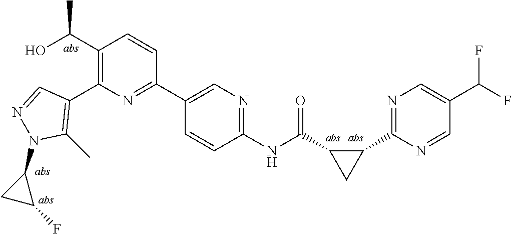 [SMILES: Cc1c(-c2nc(-c3ccc(NC(=O)[C@H]4C[C@H]4c4ncc(C(F)F)cn4)nc3)ccc2[C@H](C)O)cnn1[C@@H]1C[C@H]1F] | LCMS-5 | 550.2 | 0.546 |
| I-404 | 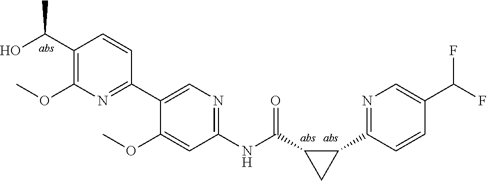 [SMILES: COc1cc(NC(=O)[C@H]2C[C@H]2c2ccc(C(F)F)cn2)ncc1-c1ccc([C@H](C)O)c(OC)n1] | LCMS-5 | 471.2 | 0.601 |
| I-405 | 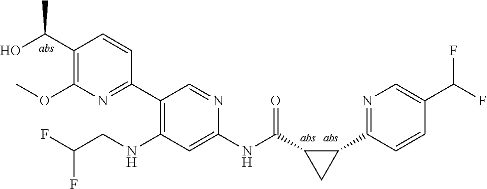 [SMILES: COc1nc(-c2cnc(NC(=O)[C@H]3C[C@H]3c3ccc(C(F)F)cn3)cc2NCC(F)F)ccc1[C@H](C)O] | LCMS-5 | 520.3 | 0.609 |
| I-406 | 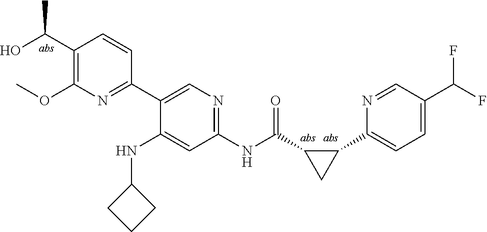 [SMILES: COc1nc(-c2cnc(NC(=O)[C@H]3C[C@H]3c3ccc(C(F)F)cn3)cc2NC2CCC2)ccc1[C@H](C)O] | LCMS-5 | 510.3 | 0.667 |
| I-407 | 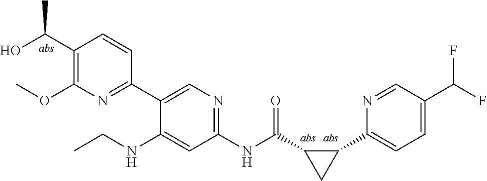 [SMILES: CCNc1cc(NC(=O)[C@H]2C[C@H]2c2ccc(C(F)F)cn2)ncc1-c1ccc([C@H](C)O)c(OC)n1] | LCMS-5 | 484.3 | 0.626 |
| I-409 | 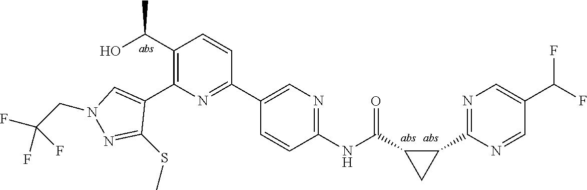 [SMILES: CSc1nn(CC(F)(F)F)cc1-c1nc(-c2ccc(NC(=O)[C@H]3C[C@H]3c3ncc(C(F)F)cn3)nc2)ccc1[C@H](C)O] | LCMS-4 | 606.3 | 0.478 |
| I-410 | 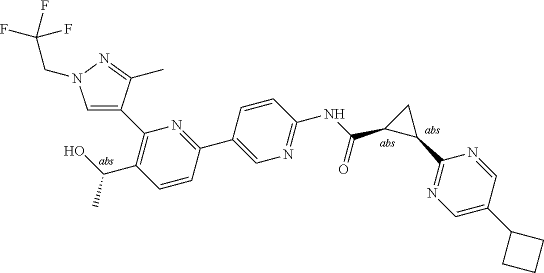 [SMILES: Cc1nn(CC(F)(F)F)cc1-c1nc(-c2ccc(NC(=O)[C@H]3C[C@H]3c3ncc(C4CCC4)cn3)nc2)ccc1[C@H](C)O] | LCMS-1 | 578.15 | 1.3 |
| I-411 | 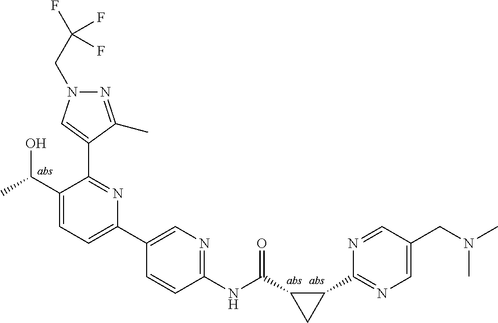 [SMILES: Cc1nn(CC(F)(F)F)cc1-c1nc(-c2ccc(NC(=O)[C@H]3C[C@H]3c3ncc(CN(C)C)cn3)nc2)ccc1[C@H](C)O] | LCMS-1 | 581.2 | 0.8 |
| I-412 | 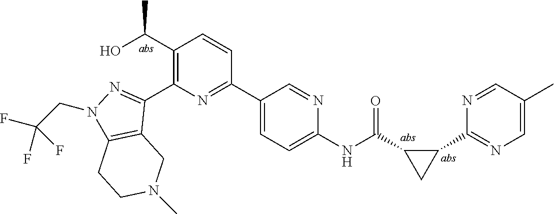 [SMILES: Cc1cnc([C@@H]2C[C@@H]2C(=O)Nc2ccc(-c3ccc([C@H](C)O)c(-c4nn(CC(F)(F)F)c5c4CN(C)CC5)n3)cn2)nc1] | LCMS-1 | 593.2 | 0.91 |
| I-413 | 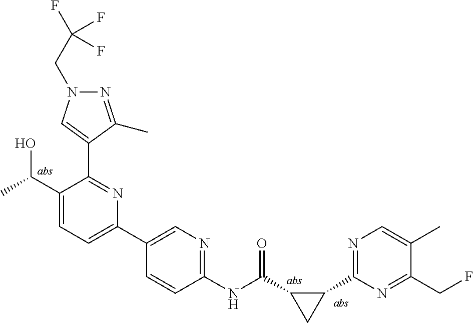 [SMILES: Cc1cnc([C@@H]2C[C@@H]2C(=O)Nc2ccc(-c3ccc([C@H](C)O)c(-c4cn(CC(F)(F)F)nc4C)n3)cn2)nc1CF] | LCMS-2 | 570.2 | 1.07 |
| I-414 | 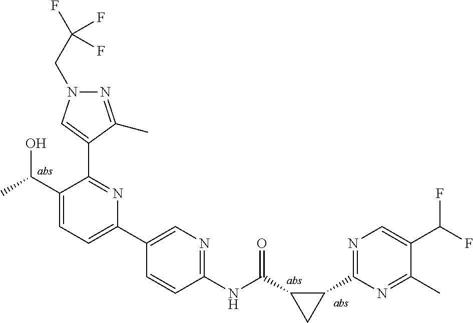 [SMILES: Cc1nn(CC(F)(F)F)cc1-c1nc(-c2ccc(NC(=O)[C@H]3C[C@H]3c3ncc(C(F)F)c(C)n3)nc2)ccc1[C@H](C)O] | LCMS-2 | 588.2 | 1.2 |
| I-415 | 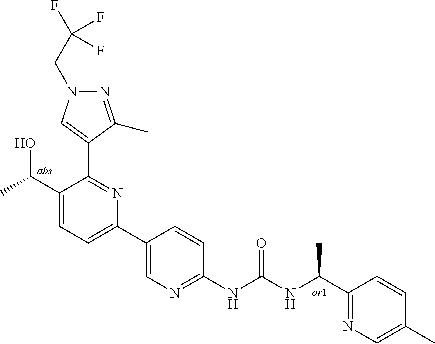 [SMILES: Cc1ccc([C@H](C)NC(=O)Nc2ccc(-c3ccc([C@H](C)O)c(-c4cn(CC(F)(F)F)nc4C)n3)cn2)nc1] | LCMS-2 | 540.25 | 1.29 |
| I-416 | 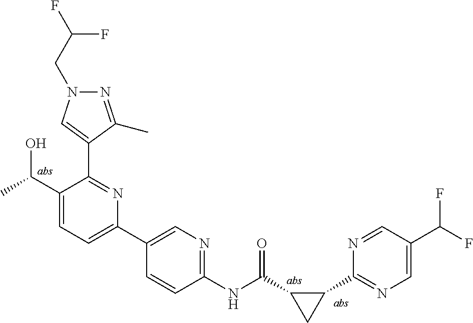 [SMILES: Cc1nn(CC(F)F)cc1-c1nc(-c2ccc(NC(=O)[C@H]3C[C@H]3c3ncc(C(F)F)cn3)nc2)ccc1[C@H](C)O] | LCMS-2 | 556.15 | 0.97 |
| I-417 | 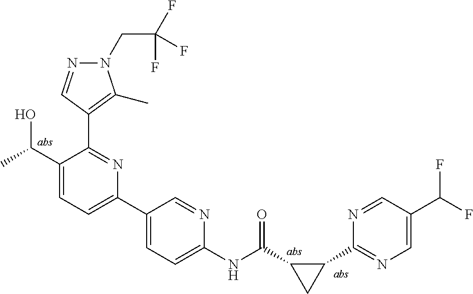 [SMILES: Cc1c(-c2nc(-c3ccc(NC(=O)[C@H]4C[C@H]4c4ncc(C(F)F)cn4)nc3)ccc2[C@H](C)O)cnn1CC(F)(F)F] | LCMS-2 | 574.2 | 1.1 |
| I-418 | 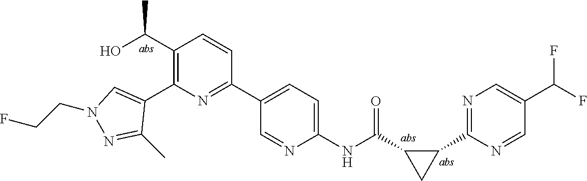 [SMILES: Cc1nn(CCF)cc1-c1nc(-c2ccc(NC(=O)[C@H]3C[C@H]3c3ncc(C(F)F)cn3)nc2)ccc1[C@H](C)O] | LCMS-2 | 538.2 | 0.82 |
| I-419 | 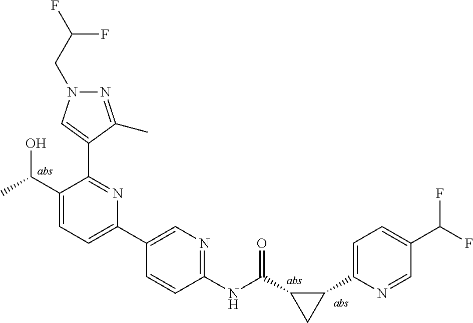 [SMILES: Cc1nn(CC(F)F)cc1-c1nc(-c2ccc(NC(=O)[C@H]3C[C@H]3c3ccc(C(F)F)cn3)nc2)ccc1[C@H](C)O] | LCMS-2 | 555.2 | 1.15 |
| I-420 | 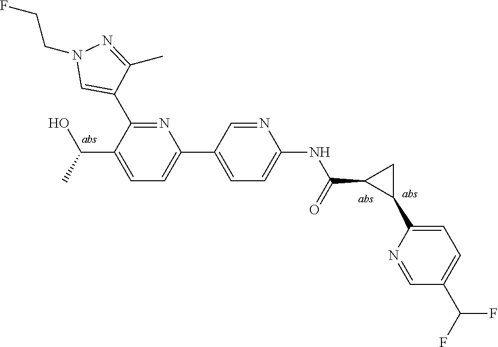 [SMILES: Cc1nn(CCF)cc1-c1nc(-c2ccc(NC(=O)[C@H]3C[C@H]3c3ccc(C(F)F)cn3)nc2)ccc1[C@H](C)O] | LCMS-2 | 537.2 | 0.96 |
| I-421 | 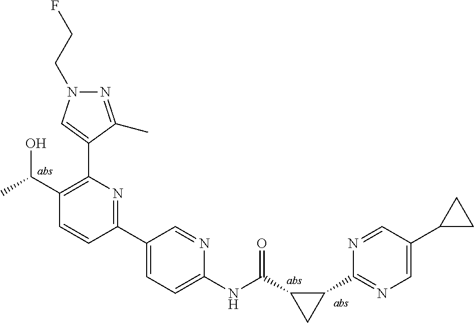 [SMILES: Cc1nn(CCF)cc1-c1nc(-c2ccc(NC(=O)[C@H]3C[C@H]3c3ncc(C4CC4)cn3)nc2)ccc1[C@H](C)O] | LCMS-2 | 528.25 | 0.93 |
| I-422 | 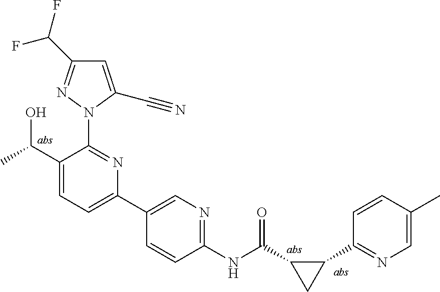 [SMILES: Cc1ccc([C@@H]2C[C@@H]2C(=O)Nc2ccc(-c3ccc([C@H](C)O)c(-n4nc(C(F)F)cc4C#N)n3)cn2)nc1] | LCMS-2 | 516.15 | 1.5 |
| I-423 | 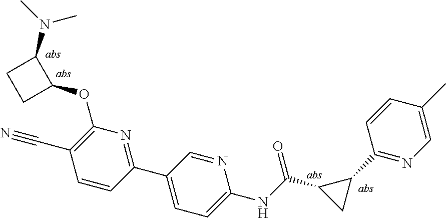 [SMILES: Cc1ccc([C@@H]2C[C@@H]2C(=O)Nc2ccc(-c3ccc(C#N)c(O[C@H]4CC[C@H]4N(C)C)n3)cn2)nc1] | LCMS-2 | 469.2 | 1.47 |
| I-424 | 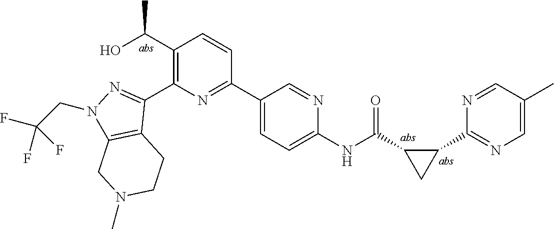 [SMILES: Cc1cnc([C@@H]2C[C@@H]2C(=O)Nc2ccc(-c3ccc([C@H](C)O)c(-c4nn(CC(F)(F)F)c5c4CCN(C)C5)n3)cn2)nc1] | LCMS-2 | 593.3 | 1.16 |
| I-425 | 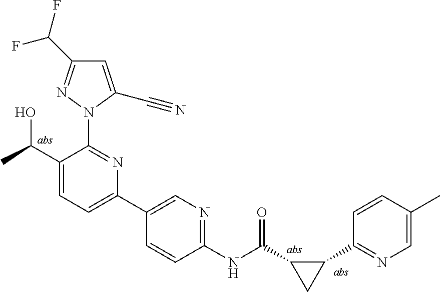 [SMILES: Cc1ccc([C@@H]2C[C@@H]2C(=O)Nc2ccc(-c3ccc([C@@H](C)O)c(-n4nc(C(F)F)cc4C#N)n3)cn2)nc1] | LCMS-2 | 516.15 | 1.5 |
| I-426 | 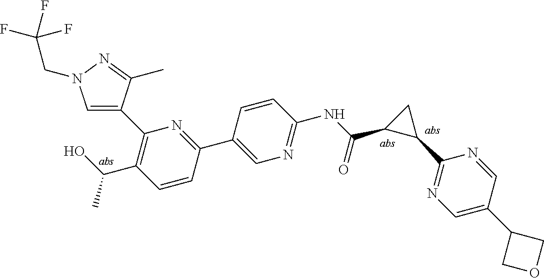 [SMILES: Cc1nn(CC(F)(F)F)cc1-c1nc(-c2ccc(NC(=O)[C@H]3C[C@H]3c3ncc(C4COC4)cn3)nc2)ccc1[C@H](C)O] | LCMS-2 | 580.2 | 0.8 |
| I-427 | 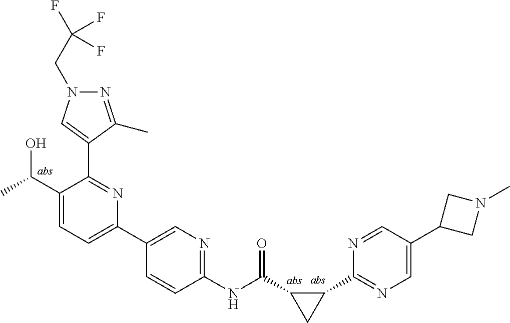 [SMILES: Cc1nn(CC(F)(F)F)cc1-c1nc(-c2ccc(NC(=O)[C@H]3C[C@H]3c3ncc(C4CN(C)C4)cn3)nc2)ccc1[C@H](C)O] | LCMS-2 | 593.25 | 0.92 |
| I-428 | 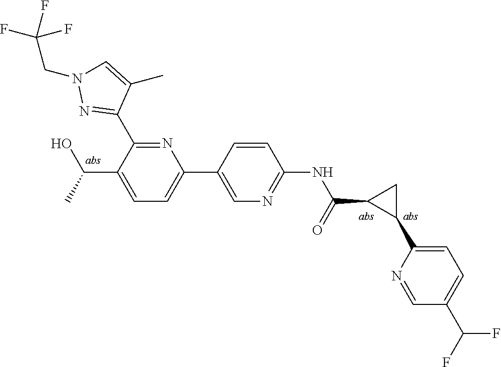 [SMILES: Cc1cn(CC(F)(F)F)nc1-c1nc(-c2ccc(NC(=O)[C@H]3C[C@H]3c3ccc(C(F)F)cn3)nc2)ccc1[C@H](C)O] | LCMS-2 | 573.25 | 1.57 |
| I-429 | 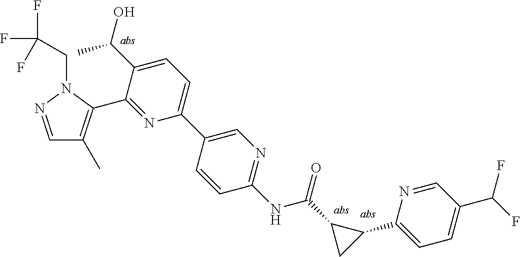 [SMILES: Cc1cnn(CC(F)(F)F)c1-c1nc(-c2ccc(NC(=O)[C@H]3C[C@H]3c3ccc(C(F)F)cn3)nc2)ccc1[C@H](C)O] | LCMS-2 | 573.2 | 1.33 |
| I-430 | 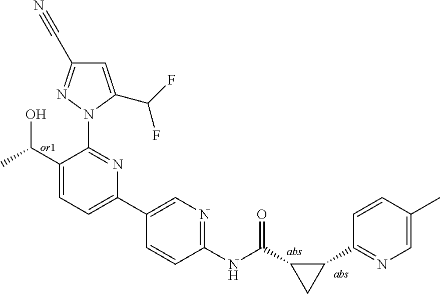 [SMILES: Cc1ccc([C@@H]2C[C@@H]2C(=O)Nc2ccc(-c3ccc([C@H](C)O)c(-n4nc(C#N)cc4C(F)F)n3)cn2)nc1] | LCMS-2 | 516.15 | 1.55 |
| I-431 | 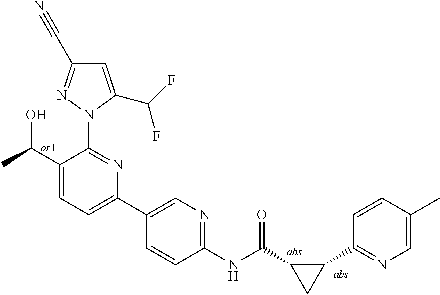 [SMILES: Cc1ccc([C@@H]2C[C@@H]2C(=O)Nc2ccc(-c3ccc([C@@H](C)O)c(-n4nc(C#N)cc4C(F)F)n3)cn2)nc1] | LCMS-2 | 516.2 | 1.54 |
| I-432 | 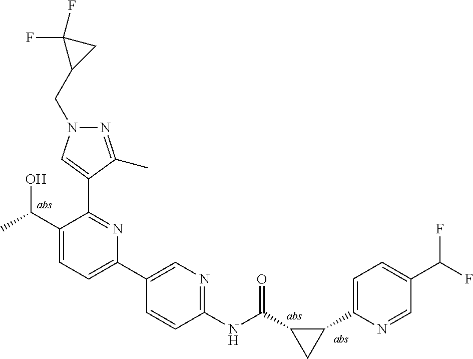 [SMILES: Cc1nn(CC2CC2(F)F)cc1-c1nc(-c2ccc(NC(=O)[C@H]3C[C@H]3c3ccc(C(F)F)cn3)nc2)ccc1[C@H](C)O] | LCMS-2 | 581.25 | 1.33 |
| I-433 | 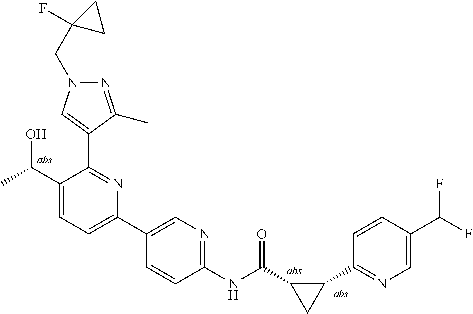 [SMILES: Cc1nn(CC2(F)CC2)cc1-c1nc(-c2ccc(NC(=O)[C@H]3C[C@H]3c3ccc(C(F)F)cn3)nc2)ccc1[C@H](C)O] | LCMS-2 | 563.25 | 1.25 |
| I-434 | 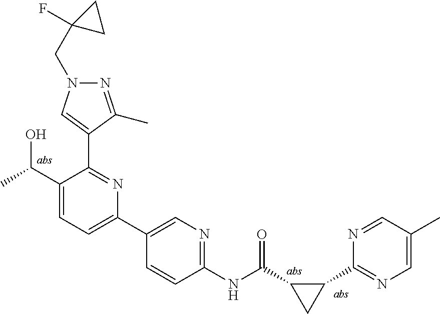 [SMILES: Cc1cnc([C@@H]2C[C@@H]2C(=O)Nc2ccc(-c3ccc([C@H](C)O)c(-c4cn(CC5(F)CC5)nc4C)n3)cn2)nc1] | LCMS-2 | 528.25 | 0.9 |
| I-435 | 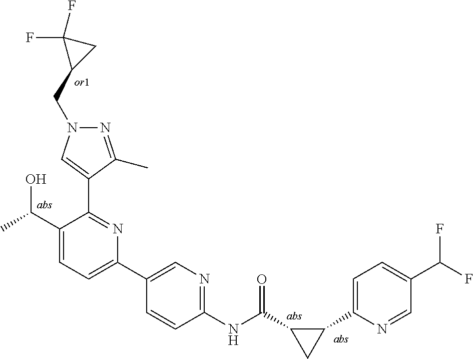 | LCMS-2 | 581.25 | 1.33 |
| I-436 | 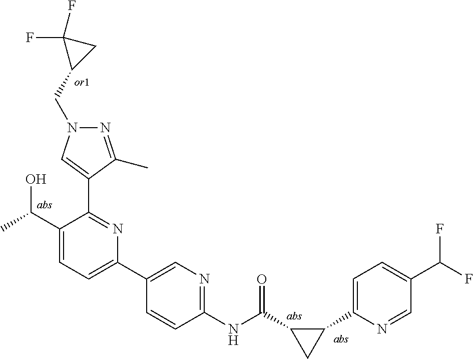 | LCMS-2 | 581.25 | 1.33 |
| I-437 | 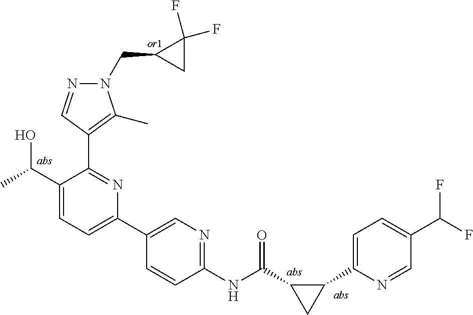 | LCMS-2 | 581.25 | 1.33 |
| I-438 | 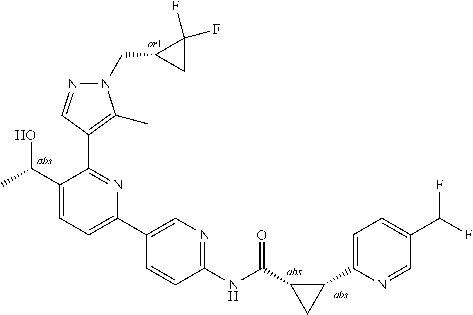 | LCMS-2 | 581.25 | 1.33 |
| I-439 | 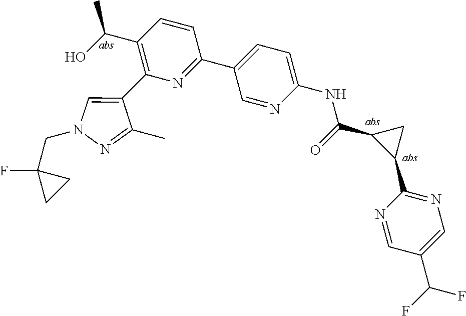 [SMILES: Cc1nn(CC2(F)CC2)cc1-c1nc(-c2ccc(NC(=O)[C@H]3C[C@H]3c3ncc(C(F)F)cn3)nc2)ccc1[C@H](C)O] | LCMS-2 | 564.25 | 1.07 |
| I-440 | 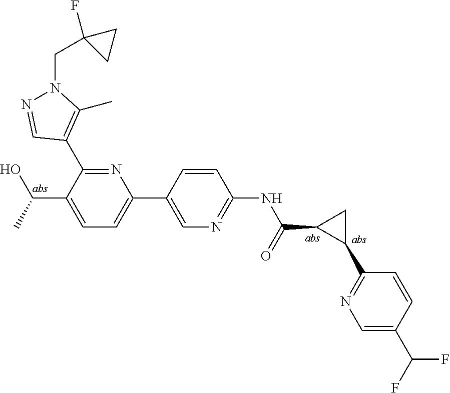 [SMILES: Cc1c(-c2nc(-c3ccc(NC(=O)[C@H]4C[C@H]4c4ccc(C(F)F)cn4)nc3)ccc2[C@H](C)O)cnn1CC1(F)CC1] | LCMS-2 | 563.25 | 1.27 |
| I-441 | 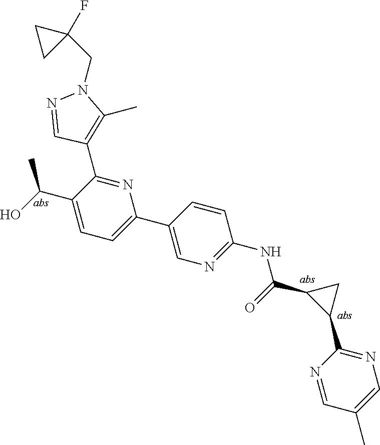 [SMILES: Cc1cnc([C@@H]2C[C@@H]2C(=O)Nc2ccc(-c3ccc([C@H](C)O)c(-c4cnn(CC5(F)CC5)c4C)n3)cn2)nc1] | LCMS-2 | 528.25 | 0.92 |
| I-442 | 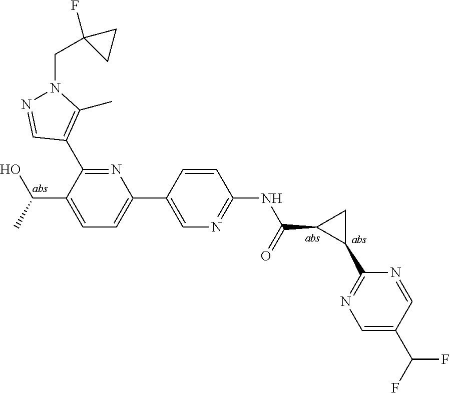 [SMILES: Cc1c(-c2nc(-c3ccc(NC(=O)[C@H]4C[C@H]4c4ncc(C(F)F)cn4)nc3)ccc2[C@H](C)O)cnn1CC1(F)CC1] | LCMS-2 | 564.25 | 1.09 |
| I-443 | 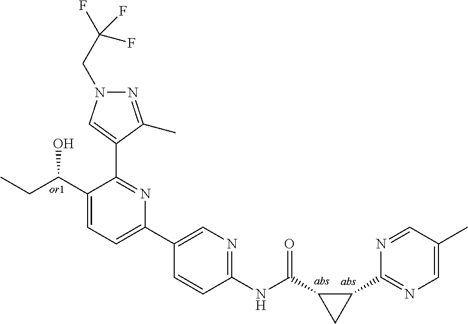 [SMILES: CC[C@H](O)c1ccc(-c2ccc(NC(=O)[C@H]3C[C@H]3c3ncc(C)cn3)nc2)nc1-c1cn(CC(F)(F)F)nc1C] | LCMS-2 | 552.25 | 1.12 |
| I-444 | 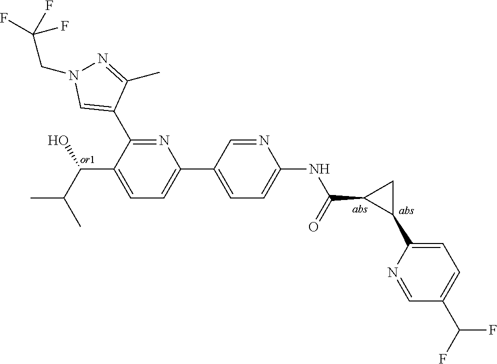 [SMILES: Cc1nn(CC(F)(F)F)cc1-c1nc(-c2ccc(NC(=O)[C@H]3C[C@H]3c3ccc(C(F)F)cn3)nc2)ccc1[C@@H](O)C(C)C] | LCMS-2 | 601.25 | 1.62 |
| I-445 | 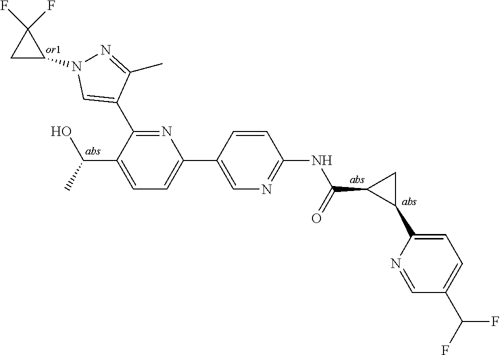 [SMILES: Cc1nn([C@@H]2CC2(F)F)cc1-c1nc(-c2ccc(NC(=O)[C@H]3C[C@H]3c3ccc(C(F)F)cn3)nc2)ccc1[C@H](C)O] | LCMS-2 | 567.2 | 1.32 |
| I-446 | 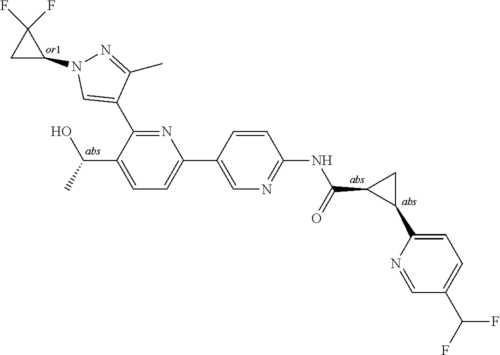 [SMILES: Cc1nn([C@H]2CC2(F)F)cc1-c1nc(-c2ccc(NC(=O)[C@H]3C[C@H]3c3ccc(C(F)F)cn3)nc2)ccc1[C@H](C)O] | LCMS-2 | 567.2 | 1.34 |
| I-447 | 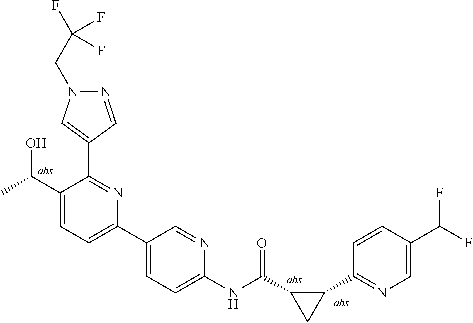 [SMILES: C[C@H](O)c1ccc(-c2ccc(NC(=O)[C@H]3C[C@H]3c3ccc(C(F)F)cn3)nc2)nc1-c1cnn(CC(F)(F)F)c1] | LCMS-2 | 559.2 | 1.39 |
| I-448 | 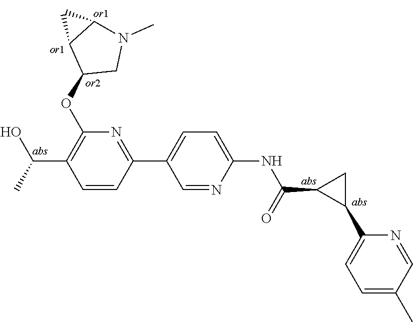 [SMILES: Cc1ccc([C@@H]2C[C@@H]2C(=O)Nc2ccc(-c3ccc([C@H](C)O)c(O[C@@H]4CN(C)[C@H]5C[C@@H]45)n3)cn2)nc1] | LCMS-2 | 484.2 | 1.28 |
| I-449 | 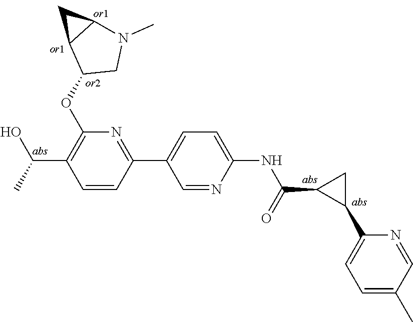 [SMILES: Cc1ccc([C@@H]2C[C@@H]2C(=O)Nc2ccc(-c3ccc([C@H](C)O)c(O[C@H]4CN(C)[C@@H]5C[C@H]45)n3)cn2)nc1] | LCMS-2 | 486.25 | 1.28 |
| I-450 | 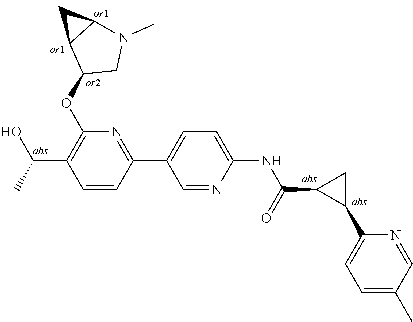 [SMILES: Cc1ccc([C@@H]2C[C@@H]2C(=O)Nc2ccc(-c3ccc([C@H](C)O)c(O[C@@H]4CN(C)[C@@H]5C[C@@H]54)n3)cn2)nc1] | LCMS-2 | 484.2 | 1.29 |
| I-451 | 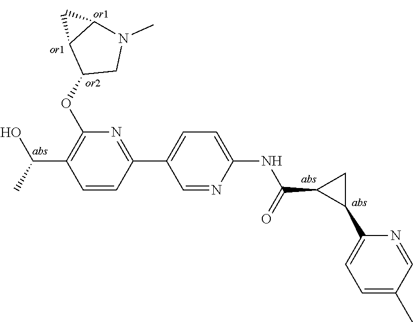 [SMILES: Cc1ccc([C@@H]2C[C@@H]2C(=O)Nc2ccc(-c3ccc([C@H](C)O)c(O[C@H]4CN(C)[C@H]5C[C@@H]45)n3)cn2)nc1] | LCMS-2 | 486.25 | 1.28 |
| I-452 | 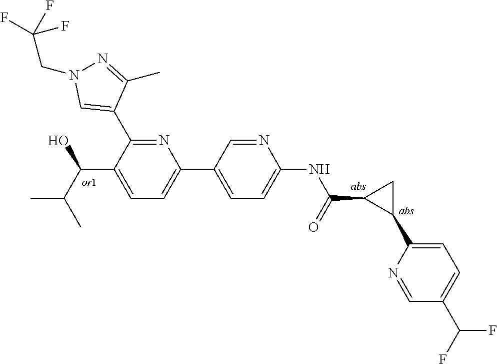 [SMILES: Cc1nn(CC(F)(F)F)cc1-c1nc(-c2ccc(NC(=O)[C@H]3C[C@H]3c3ccc(C(F)F)cn3)nc2)ccc1[C@H](O)C(C)C] | LCMS-2 | 601.25 | 1.63 |
| I-453 | 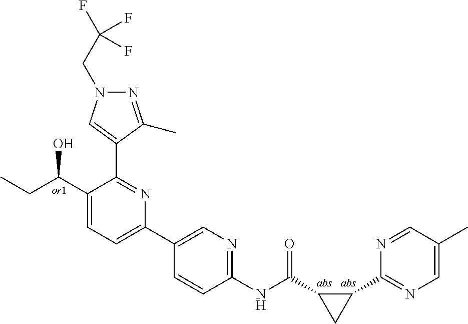 [SMILES: CC[C@@H](O)c1ccc(-c2ccc(NC(=O)[C@H]3C[C@H]3c3ncc(C)cn3)nc2)nc1-c1cn(CC(F)(F)F)nc1C] | LCMS-2 | 552.25 | 1.11 |
| I-454 | 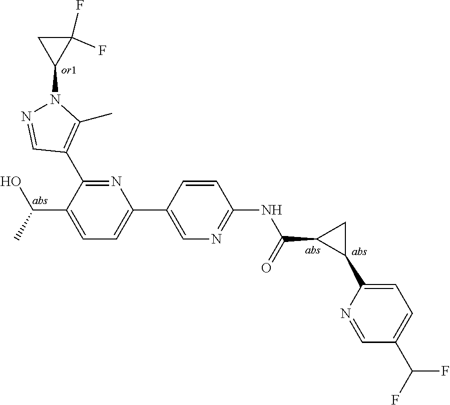 [SMILES: Cc1c(-c2nc(-c3ccc(NC(=O)[C@H]4C[C@H]4c4ccc(C(F)F)cn4)nc3)ccc2[C@H](C)O)cnn1[C@H]1CC1(F)F] | LCMS-2 | 567.25 | 1.29 |
| I-455 | 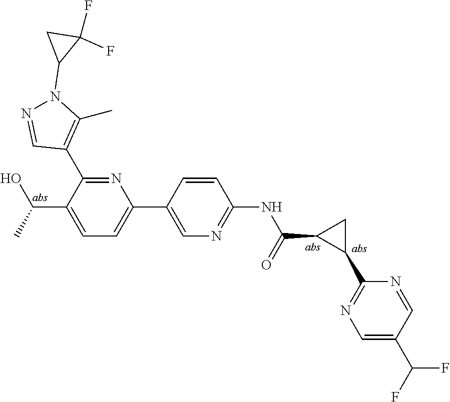 [SMILES: Cc1c(-c2nc(-c3ccc(NC(=O)[C@H]4C[C@H]4c4ncc(C(F)F)cn4)nc3)ccc2[C@H](C)O)cnn1C1CC1(F)F] | LCMS-2 | 567.25 | 1.3 |
| I-456 | 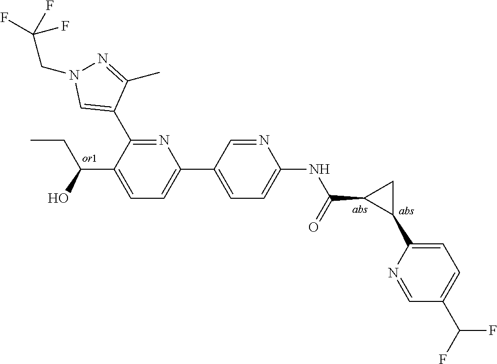 [SMILES: CC[C@H](O)c1ccc(-c2ccc(NC(=O)[C@H]3C[C@H]3c3ccc(C(F)F)cn3)nc2)nc1-c1cn(CC(F)(F)F)nc1C] | LCMS-2 | 587.25 | 1.46 |
| I-457 | 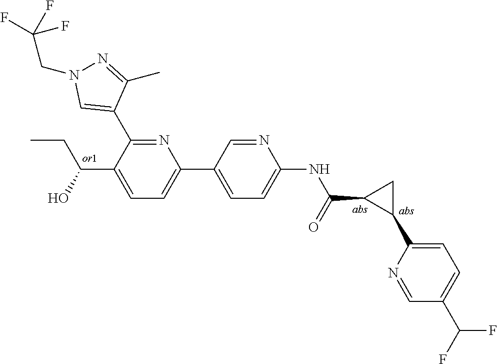 [SMILES: CC[C@@H](O)c1ccc(-c2ccc(NC(=O)[C@H]3C[C@H]3c3ccc(C(F)F)cn3)nc2)nc1-c1cn(CC(F)(F)F)nc1C] | LCMS-2 | 587.25 | 1.45 |
| I-458 | 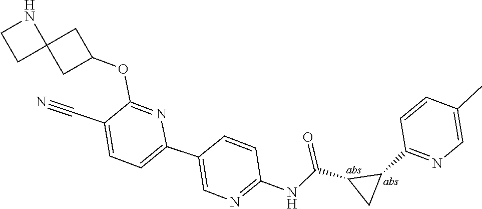 [SMILES: Cc1ccc([C@@H]2C[C@@H]2C(=O)Nc2ccc(-c3ccc(C#N)c(OC4CC5(CCN5)C4)n3)cn2)nc1] | LCMS-3 | 467.15 | 0.73 |
| I-459 | 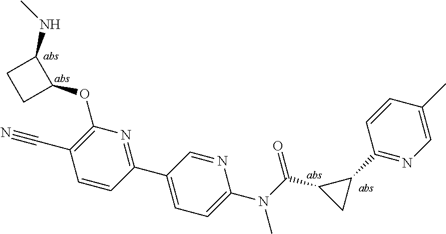 [SMILES: CN[C@@H]1CC[C@@H]1Oc1nc(-c2ccc(N(C)C(=O)[C@H]3C[C@H]3c3ccc(C)cn3)nc2)ccc1C#N] | LCMS-3 | 469.2 | 0.57 |
| I-460 | 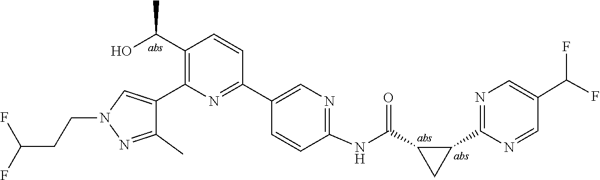 [SMILES: Cc1nn(CCC(F)F)cc1-c1nc(-c2ccc(NC(=O)[C@H]3C[C@H]3c3ncc(C(F)F)cn3)nc2)ccc1[C@H](C)O] | LCMS-4 | 570.3 | 0.448 |
| I-461 | 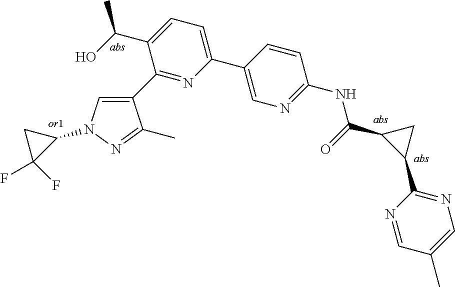 [SMILES: Cc1cnc([C@@H]2C[C@@H]2C(=O)Nc2ccc(-c3ccc([C@H](C)O)c(-c4cn([C@H]5CC5(F)F)nc4C)n3)cn2)nc1] | LCMS-4 | 532.3 | 0.436 |
| I-462 | 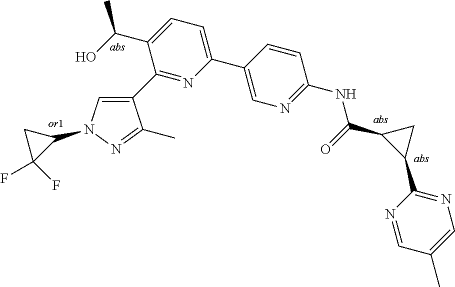 | LCMS-4 | 532.3 | 0.431 |
| I-463 | 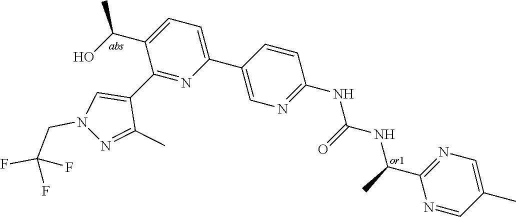 [SMILES: Cc1cnc([C@@H](C)NC(=O)Nc2ccc(-c3ccc([C@H](C)O)c(-c4cn(CC(F)(F)F)nc4C)n3)cn2)nc1] | LCMS-4 | 541.2 | 0.442 |
| I-464 | 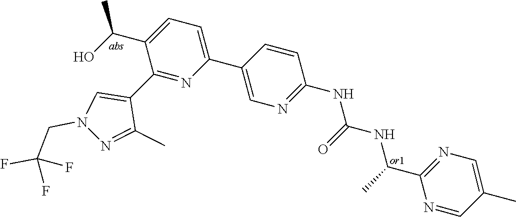 [SMILES: Cc1cnc([C@H](C)NC(=O)Nc2ccc(-c3ccc([C@H](C)O)c(-c4cn(CC(F)(F)F)nc4C)n3)cn2)nc1] | LCMS-4 | 541.3 | 0.445 |
| I-465 | 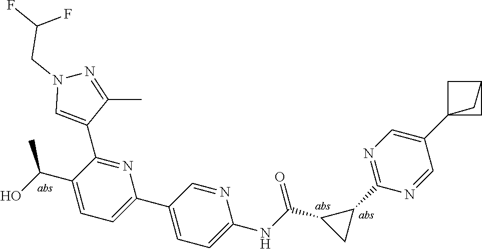 [SMILES: Cc1nn(CC(F)F)cc1-c1nc(-c2ccc(NC(=O)[C@H]3C[C@H]3c3ncc(C45CC(C4)C5)cn3)nc2)ccc1[C@H](C)O] | LCMS-4 | 572.3 | 0.461 |
| I-466 | 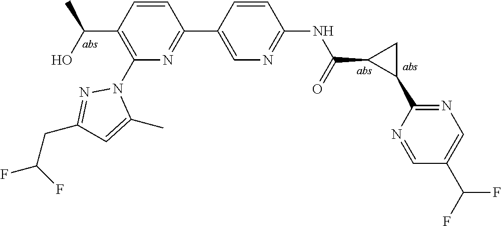 [SMILES: Cc1cc(CC(F)F)nn1-c1nc(-c2ccc(NC(=O)[C@H]3C[C@H]3c3ncc(C(F)F)cn3)nc2)ccc1[C@H](C)O] | LCMS-4 | 556.3 | 0.497 |
| I-467 | 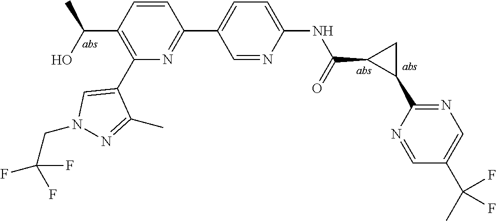 [SMILES: Cc1nn(CC(F)(F)F)cc1-c1nc(-c2ccc(NC(=O)[C@H]3C[C@H]3c3ncc(C(C)(F)F)cn3)nc2)ccc1[C@H](C)O] | LCMS-4 | 588.1 | 0.48 |
| I-468 | 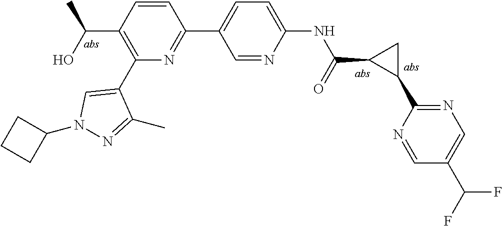 [SMILES: Cc1nn(C2CCC2)cc1-c1nc(-c2ccc(NC(=O)[C@H]3C[C@H]3c3ncc(C(F)F)cn3)nc2)ccc1[C@H](C)O] | LCMS-4 | 548.3 | 0.426 |
| I-469 | 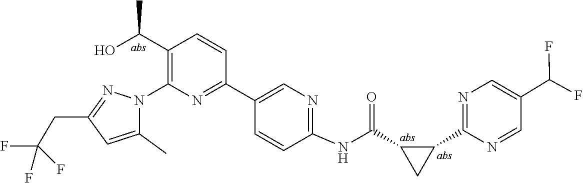 [SMILES: Cc1cc(CC(F)(F)F)nn1-c1nc(-c2ccc(NC(=O)[C@H]3C[C@H]3c3ncc(C(F)F)cn3)nc2)ccc1[C@H](C)O] | LCMS-4 | 574.2 | 0.515 |
| I-470 | 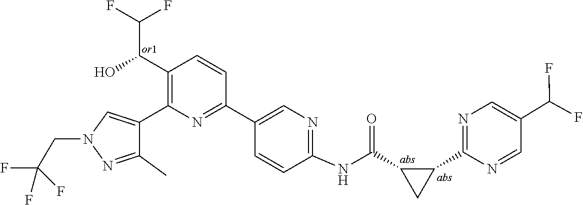 [SMILES: Cc1nn(CC(F)(F)F)cc1-c1nc(-c2ccc(NC(=O)[C@H]3C[C@H]3c3ncc(C(F)F)cn3)nc2)ccc1[C@H](O)C(F)F] | LCMS-4 | 610.3 | 0.488 |
| I-471 | 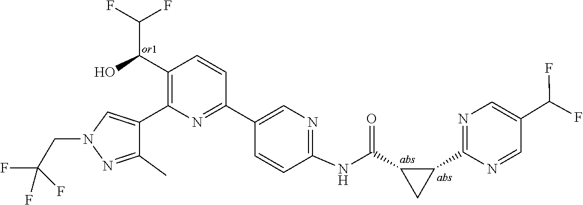 [SMILES: Cc1nn(CC(F)(F)F)cc1-c1nc(-c2ccc(NC(=O)[C@H]3C[C@H]3c3ncc(C(F)F)cn3)nc2)ccc1[C@@H](O)C(F)F] | LCMS-4 | 610.3 | 0.487 |
| I-472 | 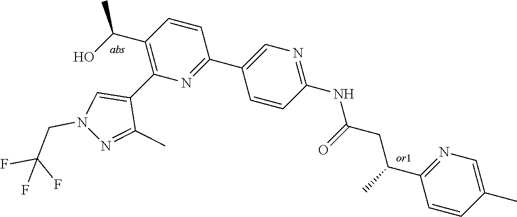 [SMILES: Cc1ccc([C@H](C)CC(=O)Nc2ccc(-c3ccc([C@H](C)O)c(-c4cn(CC(F)(F)F)nc4C)n3)cn2)nc1] | LCMS-4 | 539.2 | 0.406 |
| I-473 | 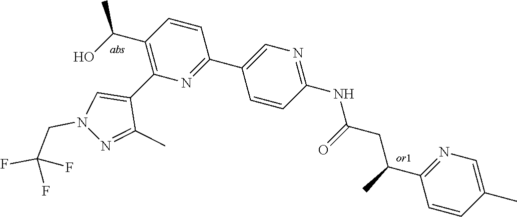 [SMILES: Cc1ccc([C@@H](C)CC(=O)Nc2ccc(-c3ccc([C@H](C)O)c(-c4cn(CC(F)(F)F)nc4C)n3)cn2)nc1] | LCMS-4 | 539.2 | 0.407 |
| I-474 | 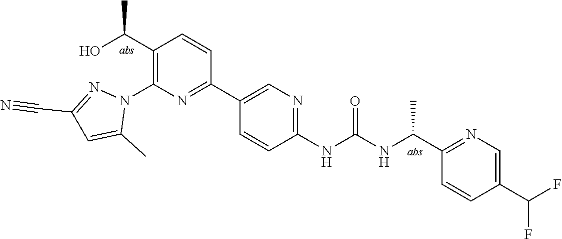 [SMILES: Cc1cc(C#N)nn1-c1nc(-c2ccc(NC(=O)N[C@H](C)c3ccc(C(F)F)cn3)nc2)ccc1[C@H](C)O] | LCMS-4 | 519.3 | 0.501 |
| I-475 | 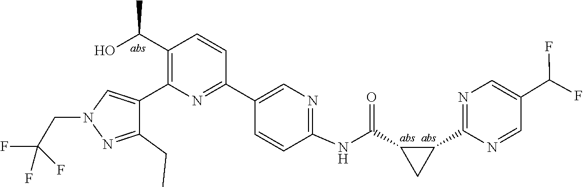 [SMILES: CCc1nn(CC(F)(F)F)cc1-c1nc(-c2ccc(NC(=O)[C@H]3C[C@H]3c3ncc(C(F)F)cn3)nc2)ccc1[C@H](C)O] | LCMS-4 | 588.2 | 0.474 |
| I-476 | 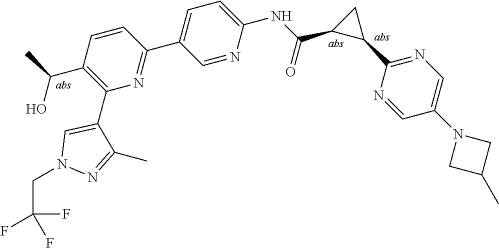 | LCMS-4 | 597.3 | 0.443 |
| I-477 | 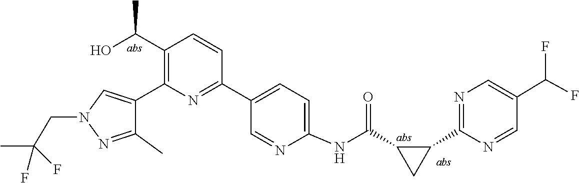 [SMILES: Cc1nn(CC(C)(F)F)cc1-c1nc(-c2ccc(NC(=O)[C@H]3C[C@H]3c3ncc(C(F)F)cn3)nc2)ccc1[C@H](C)O] | LCMS-4 | 570.1 | 0.453 |
| I-478 | 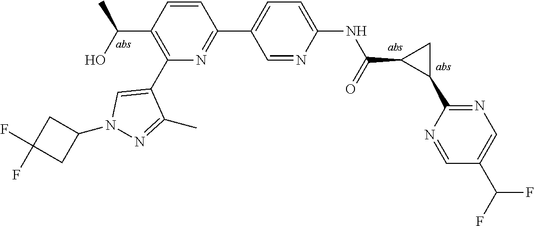 [SMILES: Cc1nn(C2CC(F)(F)C2)cc1-c1nc(-c2ccc(NC(=O)[C@H]3C[C@H]3c3ncc(C(F)F)cn3)nc2)ccc1[C@H](C)O] | LCMS-4 | 582.3 | 0.463 |
| I-479 | 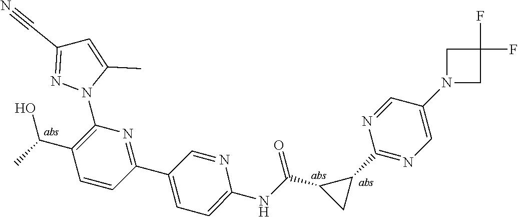 | LCMS-4 | 558.2 | 0.471 |
| I-480 | 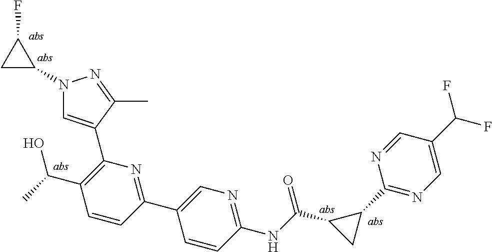 | LCMS-4 | 599.2 | 0.422 |
| I-481 | 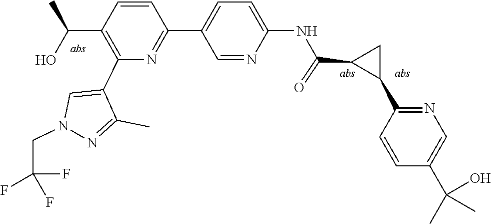 [SMILES: Cc1nn(CC(F)(F)F)cc1-c1nc(-c2ccc(NC(=O)[C@H]3C[C@H]3c3ccc(C(C)(C)O)cn3)nc2)ccc1[C@H](C)O] | LCMS-4 | 581.4 | 0.407 |
| I-482 | 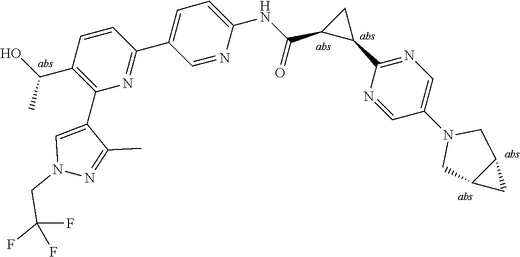 [SMILES: Cc1nn(CC(F)(F)F)cc1-c1nc(-c2ccc(NC(=O)[C@H]3C[C@H]3c3ncc(N4C[C@H]5C[C@H]5C4)cn3)nc2)ccc1[C@H](C)O] | LCMS-4 | 605.4 | 0.459 |
| I-483 | 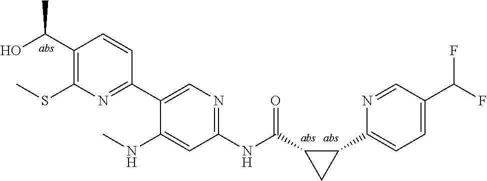 [SMILES: CNc1cc(NC(=O)[C@H]2C[C@H]2c2ccc(C(F)F)cn2)ncc1-c1ccc([C@H](C)O)c(SC)n1] | LCMS-4 | 486.2 | 0.463 |
| I-484 | 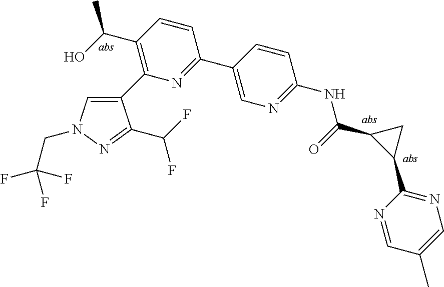 [SMILES: Cc1cnc([C@@H]2C[C@@H]2C(=O)Nc2ccc(-c3ccc([C@H](C)O)c(-c4cn(CC(F)(F)F)nc4C(F)F)n3)cn2)nc1] | LCMS-5 | 574.2 | 0.565 |
| I-485 | 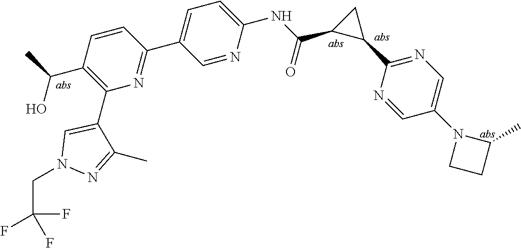 [SMILES: Cc1nn(CC(F)(F)F)cc1-c1nc(-c2ccc(NC(=O)[C@H]3C[C@H]3c3ncc(N4CC[C@H]4C)cn3)nc2)ccc1[C@H](C)O] | LCMS-5 | 593.3 | 0.534 |
| I-486 | 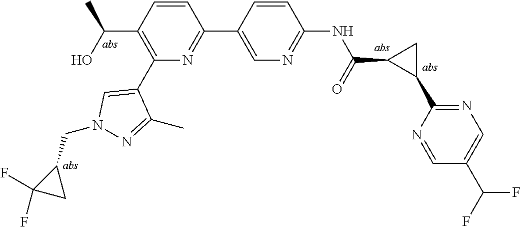 [SMILES: Cc1nn(C[C@@H]2CC2(F)F)cc1-c1nc(-c2ccc(NC(=O)[C@H]3C[C@H]3c3ncc(C(F)F)cn3)nc2)ccc1[C@H](C)O] | LCMS-5 | 582.3 | 0.565 |
| I-487 | 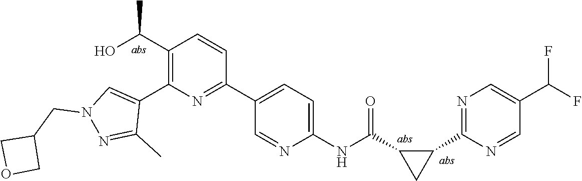 [SMILES: Cc1nn(CC2COC2)cc1-c1nc(-c2ccc(NC(=O)[C@H]3C[C@H]3c3ncc(C(F)F)cn3)nc2)ccc1[C@H](C)O] | LCMS-5 | 562.2 | 0.493 |
| I-488 | 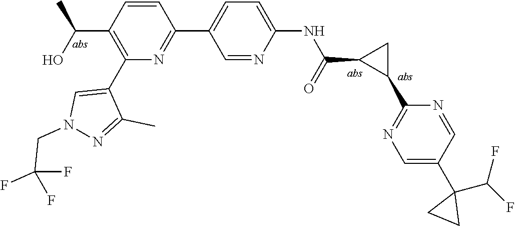 [SMILES: Cc1nn(CC(F)(F)F)cc1-c1nc(-c2ccc(NC(=O)[C@H]3C[C@H]3c3ncc(C4(C(F)F)CC4)cn3)nc2)ccc1[C@H](C)O] | LCMS-5 | 614.3 | 0.576 |
| I-489 | 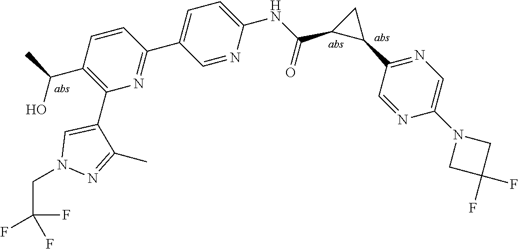 [SMILES: Cc1nn(CC(F)(F)F)cc1-c1nc(-c2ccc(NC(=O)[C@H]3C[C@H]3c3cnc(N4CC(F)(F)C4)cn3)nc2)ccc1[C@H](C)O] | LCMS-5 | 615.2 | 0.555 |
| I-490 | 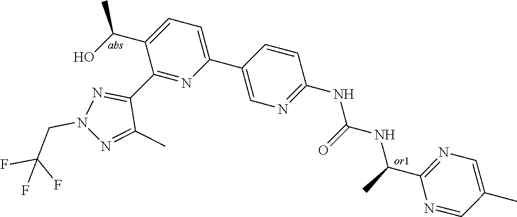 [SMILES: Cc1cnc([C@@H](C)NC(=O)Nc2ccc(-c3ccc([C@H](C)O)c(-c4nn(CC(F)(F)F)nc4C)n3)cn2)nc1] | LCMS-5 | 542.3 | 0.602 |
| I-491 | 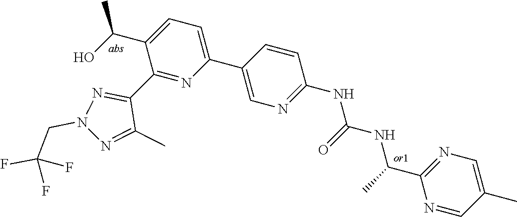 | LCMS-5 | 542.5 | 0.599 |
| I-492 | 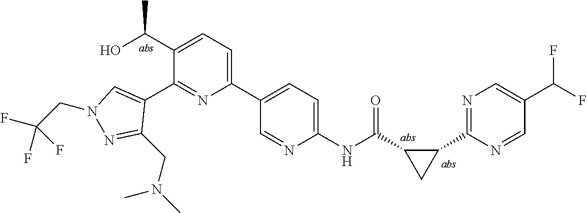 [SMILES: C[C@H](O)c1ccc(-c2ccc(NC(=O)[C@H]3C[C@H]3c3ncc(C(F)F)cn3)nc2)nc1-c1cn(CC(F)(F)F)nc1CN(C)C] | LCMS-5 | 616.3 | 0.595 |
| I-493 | 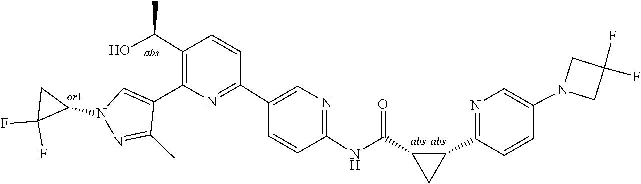 [SMILES: Cc1nn([C@H]2CC2(F)F)cc1-c1nc(-c2ccc(NC(=O)[C@H]3C[C@H]3c3ccc(N4CC(F)(F)C4)cn3)nc2)ccc1[C@H](C)O] | LCMS-5 | 608.3 | 0.601 |
| I-494 | 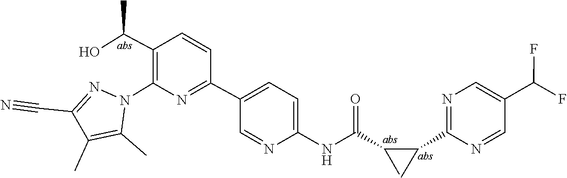 | LCMS-5 | 531.3 | 0.59 |

### Example 43: Biochemical Assays

### SIK2-FLAG (1 mM ATP) ADP-Glo Assay Protocol (Assay A)

Reagents were procured and 1× assay buffer was prepared as above except for the AMARAtide substrate and the human recombinant SIK2 (SIK2-FLAG). AMARAtide was purchased from Biopeptide (San Diego, CA) and SIK2-FLAG (UniProt ID Q9HOK1) (G13-L350) was codon optimized for expression in SF21 cells and lysed in a buffer containing 50 mM Tris-HCl (pH 7.5), 500 mM NaCl, 5% glycerol, 0.5% Tween-20, 1 mM TCEP and purified over Glutathione Sepharose FF cartridge and Size Exclusion Chromatography (HiLoad 16/600 Superdex 200pg) into a final storage buffer of 50 mM Tris-HCl (pH 7.5), 300 mM NaCl, 10% glycerol, 1 mM TCEP. The final protein, stored at −80° C., contains a single Glycine cloning artifact at the N-terminus and retains a c-terminal FLAG tag and consists of an amino acid sequence of SEQ ID NO: 1. Compounds (60 nL) in 100% DMSO were added to the assay plate (AlphaPlate-384, Cat #6005359 Perkin Elmer) using a LabCyte Echo 550. Similarly, DMSO (60 nL) was added to maximum signal control (MAX) wells and a reference compound producing 100% inhibition was added to minimum signal control (MIN) wells. SIK2 was diluted to 2× final concentration in 1× assay buffer and 3 μL was added to the compounds/DMSO in the assay plate. After incubation for 20 minutes at RT, 3 μL substrate mix containing 2×ATP and 2× AMARAtide in 1× assay buffer was added to the assay plate. After incubation for 60 minutes, the reaction was stopped by the addition of the ADP-Glo Kit components as directed and the relative luminescence units (RLU) were read on an Envision 2104. The final concentrations of the assay components were 3 nM SIK2-FLAG, 50 μM AMARAtide, 1 mM ATP, and 1% DMSO.

### Inhibition Calculation:

$$\% INH = ( RLU MAX - RLU sample ) / ( RLU MAX - RLU MIN ) \times 100$$

Where RLU=relative luminescence units, sample=signal in sample well, and MIN and MAX are the minimum and maximum signal controls, respectively.

### Four-Parameter IC₅₀ Fit:

$$Y = Bottom + ( Top - Bottom ) / ( 1 + ( {IC}_{50} / X ) \hat{} Hill Slope )$$

Where top and bottom are the normally allowed to float, but may be fixed at 100 or 0, respectively, in a 3-parameter fit. Y is the % inhibition and X is the compound concentration.

Table 3 shows the IC₅₀ of selected compounds of this disclosure in the above assays. Compound activity designations have the following values: A (<10 nM); B (10 nM to <100 nM); C (100 nM to <500 nM); D (>500 nM); and ** (not tested).

TABLE 3
Biochemical Assays Results

| I-# | Assay A |
|---|---|
| I-1 | A |
| I-2 | A |
| I-3 | A |
| I-4 | A |
| I-5 | A |
| I-6 | A |
| I-7 | A |
| I-8 | A |
| I-9 | A |
| I-10 | A |
| I-11 | A |
| I-12 | A |
| I-13 | A |
| I-14 | A |
| I-15 | A |
| I-16 | A |
| I-17 | A |
| I-18 | A |
| I-19 | A |
| I-20 | A |
| I-21 | A |
| I-22 | A |
| I-23 | A |
| I-24 | A |
| I-25 | A |
| I-26 | A |
| I-27 | A |
| I-28 | A |
| I-29 | A |
| I-30 | A |
| I-31 | A |
| I-32 | A |
| I-33 | A |
| I-34 | A |
| I-35 | A |
| I-36 | A |
| I-37 | A |
| I-38 | A |
| I-39 | A |
| I-40 | B |
| I-41 | B |
| I-42 | B |
| I-43 | B |
| I-44 | B |
| I-45 | B |
| I-46 | B |
| I-47 | B |
| I-48 | B |
| I-49 | B |
| I-50 | B |
| I-51 | B |
| I-52 | B |
| I-53 | B |
| I-54 | B |
| I-55 | B |
| I-56 | B |
| I-57 | B |
| I-58 | B |
| I-59 | B |
| I-60 | B |
| I-61 | B |
| I-62 | B |
| I-63 | B |
| I-64 | B |
| I-65 | B |
| I-66 | B |
| I-67 | B |
| I-68 | B |
| I-69 | B |
| I-70 | B |
| I-71 | B |
| I-72 | B |
| I-73 | B |
| I-74 | B |
| I-75 | B |
| I-76 | B |
| I-77 | B |
| I-78 | B |
| I-79 | B |
| I-80 | B |
| I-81 | B |
| I-82 | C |
| I-83 | C |
| I-84 | C |
| I-85 | C |
| I-86 | C |
| I-87 | C |
| I-88 | C |
| I-89 | C |
| I-90 | C |
| I-91 | C |
| I-92 | C |
| I-93 | C |
| I-94 | C |
| I-95 | C |
| I-96 | C |
| I-97 | C |
| I-98 | C |
| I-99 | C |
| I-100 | C |
| I-101 | C |
| I-102 | C |
| I-103 | C |
| I-104 | C |
| I-105 | C |
| I-106 | C |
| I-107 | C |
| I-108 | C |
| I-109 | C |
| I-110 | C |
| I-111 | C |
| I-112 | C |
| I-113 | C |
| I-114 | C |
| I-115 | C |
| I-116 | C |
| I-117 | C |
| I-118 | C |
| I-119 | C |
| I-120 | C |
| I-121 | C |
| I-122 | C |
| I-123 | C |
| I-124 | C |
| I-125 | C |
| I-126 | C |
| I-127 | C |
| I-128 | C |
| I-129 | C |
| I-130 | C |
| I-131 | C |
| I-132 | C |
| I-133 | C |
| I-134 | C |
| I-135 | C |
| I-136 | C |
| I-137 | D |
| I-138 | D |
| I-139 | D |
| I-140 | D |
| I-141 | D |
| I-142 | D |
| I-143 | D |
| I-144 | D |
| I-145 | D |
| I-146 | D |
| I-147 | D |
| I-148 | D |
| I-149 | D |
| I-150 | A |
| I-151 | B |
| I-152 | A |
| I-153 | A |
| I-154 | C |
| I-155 | A |
| I-156 | A |
| I-157 | B |
| I-158 | B |
| I-159 | B |
| I-160 | B |
| I-161 | D |
| I-162 | A |
| I-163 | A |
| I-164 | A |
| I-165 | A |
| I-166 | B |
| I-167 | A |
| I-168 | B |
| I-169 | B |
| I-170 | A |
| I-171 | A |
| I-172 | C |
| I-173 | D |
| I-174 | A |
| I-175 | D |
| I-176 | D |
| I-177 | D |
| I-178 | D |
| I-179 | B |
| I-180 | D |
| I-181 | C |
| I-182 | C |
| I-183 | A |
| I-184 | B |
| I-185 | A |
| I-186 | B |
| I-187 | C |
| I-188 | B |
| I-189 | C |
| I-190 | C |
| I-191 | A |
| I-192 | C |
| I-193 | B |
| I-194 | A |
| I-195 | A |
| I-196 | C |
| I-197 | C |
| I-198 | C |
| I-199 | A |
| I-200 | C |
| I-201 | A |
| I-202 | A |
| I-203 | A |
| I-204 | A |
| I-205 | A |
| I-206 | C |
| I-207 | C |
| I-208 | C |
| I-209 | B |
| I-210 | C |
| I-211 | C |
| I-212 | C |
| I-213 | C |
| I-214 | C |
| I-215 | C |
| I-216 | C |
| I-217 | C |
| I-218 | C |
| I-219 | A |
| I-220 | C |
| I-221 | B |
| I-222 | C |
| I-223 | C |
| I-224 | A |
| I-225 | C |
| I-226 | A |
| I-227 | B |
| I-228 | C |
| I-229 | C |
| I-230 | C |
| I-231 | B |
| I-232 | C |
| I-233 | B |
| I-234 | A |
| I-235 | D |
| I-236 | C |
| I-237 | C |
| I-238 | C |
| I-239 | C |
| I-240 | B |
| I-241 | C |
| I-242 | B |
| I-243 | B |
| I-244 | C |
| I-245 | C |
| I-246 | C |
| I-247 | A |
| I-248 | C |
| I-249 | C |
| I-250 | C |
| I-251 | C |
| I-252 | B |
| I-253 | A |
| I-254 | B |
| I-255 | C |
| I-256 | C |
| I-257 | C |
| I-258 | C |
| I-259 | C |
| I-260 | B |
| I-261 | C |
| I-262 | C |
| I-263 | A |
| I-264 | A |
| I-265 | A |
| I-266 | C |
| I-267 | C |
| I-268 | A |
| I-269 | B |
| I-270 | C |
| I-271 | C |
| I-272 | C |
| I-273 | C |
| I-274 | A |
| I-275 | B |
| I-276 | C |
| I-277 | A |
| I-278 | C |
| I-279 | C |
| I-280 | C |
| I-281 | C |
| I-282 | C |
| I-283 | C |
| I-284 | C |
| I-285 | D |
| I-286 | C |
| I-287 | A |
| I-288 | B |
| I-289 | A |
| I-290 | B |
| I-291 | A |
| I-292 | C |
| I-293 | C |
| I-294 | C |
| I-295 | C |
| I-296 | B |
| I-297 | A |
| I-298 | C |
| I-299 | A |
| I-300 | C |
| I-301 | C |
| I-302 | C |
| I-303 | A |
| I-304 | B |
| I-305 | C |
| I-306 | B |
| I-307 | C |
| I-308 | C |
| I-309 | A |
| I-310 | C |
| I-311 | C |
| I-312 | C |
| I-313 | A |
| I-314 | B |
| I-315 | B |
| I-316 | A |
| I-317 | C |
| I-318 | C |
| I-319 | A |
| I-320 | C |
| I-321 | C |
| I-322 | B |
| I-323 | C |
| I-324 | B |
| I-325 | C |
| I-326 | A |
| I-327 | A |
| I-328 | A |
| I-329 | B |
| I-330 | A |
| I-331 | A |
| I-332 | B |
| I-333 | A |
| I-334 | A |
| I-335 | B |
| I-336 | C |
| I-337 | C |
| I-338 | A |
| I-339 | A |
| I-340 | C |
| I-341 | C |
| I-342 | C |
| I-343 | C |
| I-344 | C |
| I-345 | C |
| I-346 | C |
| I-347 | A |
| I-348 | B |
| I-349 | A |
| I-350 | A |
| I-351 | B |
| I-352 | B |
| I-353 | D |
| I-354 | B |
| I-355 | A |
| I-356 | C |
| I-357 | B |
| I-358 | A |
| I-359 | A |
| I-360 | C |
| I-361 | C |
| I-362 | A |
| I-363 | C |
| I-364 | C |
| I-365 | A |
| I-366 | B |
| I-367 | D |
| I-368 | C |
| I-369 | C |
| I-370 | A |
| I-371 | A |
| I-372 | C |
| I-373 | B |
| I-374 | B |
| I-375 | C |
| I-376 | C |
| I-377 | A |
| I-378 | C |
| I-379 | B |
| I-380 | B |
| I-381 | A |
| I-382 | A |
| I-383 | A |
| I-384 | A |
| I-385 | A |
| I-386 | A |
| I-387 | A |
| I-388 | B |
| I-389 | A |
| I-390 | C |
| I-391 | B |
| I-392 | B |
| I-393 | C |
| I-394 | B |
| I-395 | B |
| I-396 | C |
| I-397 | B |
| I-398 | C |
| I-399 | C |
| I-400 | B |
| I-401 | C |
| I-402 | B |
| I-403 | C |
| I-404 | D |
| I-405 | B |
| I-406 | A |
| I-407 | A |
| I-409 | A |
| I-410 | A |
| I-411 | C |
| I-412 | A |
| I-413 | B |
| I-414 | A |
| I-415 | B |
| I-416 | B |
| I-417 | B |
| I-418 | B |
| I-419 | A |
| I-420 | A |
| I-421 | A |
| I-422 | C |
| I-423 | C |
| I-424 | A |
| I-425 | A |
| I-426 | B |
| I-427 | C |
| I-428 | A |
| I-429 | C |
| I-430 | C |
| I-431 | A |
| I-432 | A |
| I-433 | A |
| I-434 | A |
| I-435 | A |
| I-436 | A |
| I-437 | B |
| I-438 | B |
| I-439 | A |
| I-440 | B |
| I-441 | C |
| I-442 | C |
| I-443 | B |
| I-444 | B |
| I-445 | B |
| I-446 | A |
| I-447 | A |
| I-448 | B |
| I-449 | C |
| I-450 | C |
| I-451 | C |
| I-452 | C |
| I-453 | C |
| I-454 | B |
| I-455 | B |
| I-456 | A |
| I-457 | C |
| I-458 | B |
| I-459 | C |
| I-460 | B |
| I-461 | B |
| I-462 | A |
| I-463 | C |
| I-464 | B |
| I-465 | A |
| I-466 | C |
| I-467 | B |
| I-468 | C |
| I-469 | C |
| I-470 | A |
| I-471 | C |
| I-472 | B |
| I-473 | C |
| I-474 | B |
| I-475 | A |
| I-476 | B |
| I-477 | A |
| I-478 | B |
| I-479 | B |
| I-480 | A |
| I-481 | A |
| I-482 | A |
| I-483 | A |
| I-484 | A |
| I-485 | A |
| I-486 | B |
| I-487 | C |
| I-488 | B |
| I-489 | B |
| I-490 | C |
| I-491 | B |
| I-492 | A |
| I-493 | A |
| I-494 | B |

While we have described a number of embodiments of this disclosure, it is apparent that our examples may be altered to provide other embodiments that utilize the compounds and methods of this disclosure. Therefore, it will be appreciated that the scope of this disclosure is to be defined by the appended claims rather than by the specific embodiments that have been represented by way of example.

1. A compound of formula I-a-1 or I-b′: 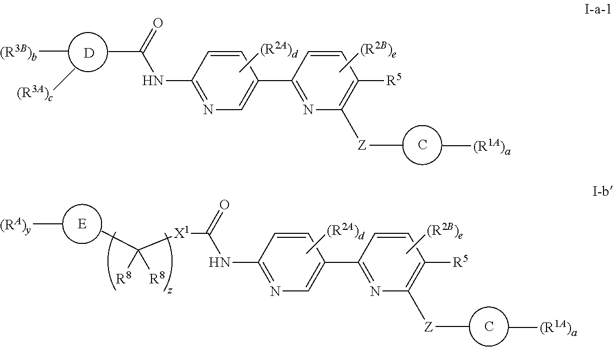
or a pharmaceutically acceptable salt thereof, wherein:
X¹ is O or NH;
d and e are each independently 0 or 1;
R^{2A} and R^{2B} are each independently selected from OH, C₁₋₃ alkyl, C₁₋₃ alkoxy, C₁₋₃ haloalkyl, C₁₋₃ hydroxyalkyl, —N(R⁴)₂, and halogen;
each R⁴ is independently selected from hydrogen and C₁₋₄ alkyl;
z is 0, 1, or 2;
each R⁸ is independently selected from hydrogen, halogen, hydroxyl, —CN, C₁₋₄ alkoxy, a 3-8 membered saturated or partially unsaturated monocyclic carbocyclic, and C₁₋₄ alkyl; or two R⁸ groups and the carbon atom to which they are attached form oxo, a spiro 3-8 membered saturated or partially unsaturated monocyclic carbocyclic ring or a spiro 3-8 membered saturated or partially unsaturated monocyclic heterocyclic ring having 1-4 heteroatoms independently selected from nitrogen, oxygen, and sulfur, wherein said spiro 3-8 membered saturated or partially unsaturated monocyclic carbocyclic ring or spiro 3-8 membered saturated or partially unsaturated monocyclic heterocyclic ring is substituted with 0, 1 or 2 substituents independently selected from halogen, C₁₋₃ alkyl, C₁₋₃ alkoxy, and C₁₋₃ haloalkyl;
R⁵ is selected from hydrogen, —CN, OH, C₁₋₄ alkyl, C₁₋₄ alkoxy, C₁₋₄ haloalkyl, C₁₋₄ hydroxyalkyl, halogen, —OR, —SR, —NR₂, —SO₂R, —SO₂NR₂, —S(O)R, —S(O)NR₂, —C(O)R, —CO₂R, —C(O)NR₂, —OC(O)R, and —OC(O)NR₂, wherein C₁₋₄ alkyl, C₁₋₄ alkoxy or C₁₋₄ hydroxyalkyl is optionally substituted with —CN —OH, or 1, 2, or 3 halogens, or
R⁵ is a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur, and is substituted with 0, 1, 2, or 3 R^{S} substituents;
each R^{S} is independently OH, C₁₋₃alkyl, C₁₋₃alkoxy, C₁₋₃haloalkyl, C₁₋₃hydroxyalkyl, —N(R⁴)₂, and halogen;
a is 0, 1, 2, or 3;
each R^{1A} is independently halogen, —CN, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or
R^{1A} is C₀₋₃alkylene-Cyc and Cyc is phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 4-12 membered saturated or partially unsaturated fused, bridged, or spiro carbocyclic ring system, and a fused, bridged or spiro 4-12 membered saturated or partially unsaturated heterocyclic ring system having 1-4 heteroatoms independently selected from nitrogen, oxygen, and sulfur, and Cyc is substituted with 0, 1, 2, or 3 substituents independently selected from C₁₋₃ alkyl, C₁₋₃ alkoxy, C₁₋₃ haloalkyl, C₁₋₃ hydroxyalkyl, halogen, —CN, —N(R⁴)₂; and a 5-6 membered heteroaryl ring having 1-4 heteroatoms independently selected from nitrogen, oxygen, and sulfur; or
R^{1A} and R^{2A} combine to form a linker having a structure of —OC₂₋₆alkylene-NH— or —C₂₋₆alkylene-NH—, wherein the N atom forms a direct bond with the pyridine ring;
Ring D is a carbon linked cyclic group selected from:
a 3-8 membered saturated or partially unsaturated monocyclic carbocyclic ring;
a 5-10 membered saturated or partially unsaturated spirocyclic ring having 0-3 heteroatoms independently selected from nitrogen, oxygen, and sulfur;
a 3-8 membered saturated or partially unsaturated monocyclic heterocyclic ring having 1-2 heteroatoms independently selected from nitrogen, oxygen, and sulfur; and
a 5-12 membered saturated or partially unsaturated bicyclic heterocyclic ring that is optionally bridged or spirocyclic having 1-4 heteroatoms independently selected from nitrogen, oxygen, and sulfur;
Ring E is a cyclic group selected from:
a 5-6 membered monocyclic heteroaromatic ring having 1-4 heteroatoms independently selected from nitrogen, oxygen, and sulfur;
phenyl;
an 8-10 membered bicyclic heteroaromatic ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur;
y is 0, 1, 2, 3, or 4;
Z is a bond, #C(R⁶)₂—[C(R⁷)₂]₀₋₂—, #C(R⁶)₂—[C(R⁷)₂]₀₋₂—O—, #O—[C(R⁷)₂]₀₋₂—, or #NR⁶—[C(R⁹)₂]₀₋₂—, wherein # is a direct bond to the pyridine ring;
Ring C is a cyclic group selected from:
phenyl;
a 3-8 membered saturated or partially unsaturated carbocyclic ring;
a 3-8 membered saturated or partially unsaturated heterocyclic ring having 1-2 heteroatoms independently selected from nitrogen, oxygen, and sulfur;
a 5-6 membered heteroaryl ring having 1-4 heteroatoms independently selected from nitrogen, oxygen, and sulfur;
a 8-10 membered bicyclic heteroaryl ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-8 membered saturated or partially unsaturated bridged bicyclic ring having 0-3 heteroatoms independently selected from nitrogen, oxygen, and sulfur;
a 5-10 membered saturated or partially unsaturated spirocyclic ring having 0-3 heteroatoms independently selected from nitrogen, oxygen, and sulfur; and
a 5-11 membered saturated or partially unsaturated bicyclic ring having 0-2 heteroatoms independently selected from nitrogen, oxygen, and sulfur; or
Ring C is absent and
(i) a is 0 and R^{2A} and Z together form —OC₂₋₆alkylen-NH—, wherein O is the Z point of attachment to the pyridine ring; or
(ii) Z is a bond to R^{1A}, and a is 1;
b is 0 or 1;
R^{3B} is halogen, oxo, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, or
R^{3B} is a 3-7 membered saturated or partially unsaturated monocyclic or bridged carbocyclic ring;
a 3-7 membered saturated or partially unsaturated monocyclic heterocyclic ring having 1-2 heteroatoms independently selected from nitrogen, oxygen, and sulfur; or a 5-6 membered heteroaryl ring having 1-4 heteroatoms independently selected from nitrogen, oxygen, and sulfur, and is substituted with 0, 1, or 2 R^{S} substituents;
c is 0 or 1;
R^{3A} is a C₀₋₆alkylene-cyclic group; wherein the cyclic group is selected from:
a 5-6 membered heteroaromatic ring having 1-4 heteroatoms independently selected from nitrogen, oxygen, and sulfur;
phenyl;
an 8-10 membered bicyclic heteroaromatic ring having 1-5 heteroatoms independently selected from nitrogen, oxygen, and sulfur;
a 7-12 membered saturated or partially unsaturated bicyclic or tricyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur;
wherein R^{3A} is substituted with 0,1, 2, 3, or 4 R^{A};
each R^{A} is independently selected from halogen; C₁₋₃ alkyl; C₂₋₆ alkenyl; C₂₋₄ alkynyl; C₁₋₃ alkoxy; C₁₋₃ haloalkyl; C₁₋₃ hydroxyalkyl; —CN; —OR; —SR; —SF₆; —C₀₋₃alkylene-NR₂; —SO₂R; —SO₂NR₂; —S(O)R; —S(O)NR₂; —C(O)R; —CO₂R; —C(O)NR₂; —C(O)N(R)OR; —OC(O)R; —OC(O)NR₂; —N(R)CO₂R; a 3-7 membered saturated or partially unsaturated monocyclic carbocyclic ring, substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur; a 5-6 membered heteroaryl ring having 1-4 heteroatoms independently selected from nitrogen, oxygen, and sulfur; a fused, bridged, or spiro 4-12 membered saturated or partially unsaturated carbocyclic ring, and a fused, bridged, or spiro 4-12 membered saturated or partially unsaturated heterocyclic ring system having 1-4 heteroatoms independently selected from nitrogen, oxygen, and sulfur, wherein said carbocyclic ring, heterocyclic ring, or heteroaryl ring is substituted with 0, 1, 2 or 3 substituents independently selected from C₁₋₃ alkyl, C₁₋₃ alkoxy, C₁₋₃ haloalkyl, C₁₋₃ hydroxyalkyl, halogen, —CN, —OR, and —NR₂; or
two R^{A} on the same carbon atom together form oxo or a spiro cyclic group selected from:
3-8 membered saturated or partially unsaturated monocyclic carbocyclic ring; and
3-8 membered saturated or partially unsaturated monocyclic heterocyclic ring having 1-2 heteroatoms independently selected from nitrogen, oxygen, and sulfur;
wherein the cyclic group formed by two R^{A} on the same carbon atom is substituted with 0,1, 2, or 3 groups independently selected from a C₁₋₆alkyl, C₁₋₆haloalkyl, C₁₋₆alkylene-CN, C₁₋₆alkylene-NR₂, C₁₋₆alkylene-OR; halogen; —CN; —NO₂; —OR;
SR; —NR₂; —S(O)₂R; —S(O)₂NR₂; —S(O)R; —S(O)NR₂; —C(O)R; —C(O)OR;
—C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
two R^{A} on adjacent atoms form a fused cyclic group selected from:
3-8 membered saturated or partially unsaturated monocyclic carbocyclic ring;
a 3-8 membered saturated or partially unsaturated monocyclic heterocyclic ring having 1-2 heteroatoms independently selected from nitrogen, oxygen, and sulfur;
phenyl; and
a 5-6 membered heteroaryl ring having 1-4 heteroatoms independently selected from nitrogen, oxygen, and sulfur;
wherein the fused cyclic group formed by two R^{A} on adjacent atoms is substituted with 0,1, 2, or 3 groups independently selected from a C₁₋₆ alkyl, C₁₋₆haloalkyl, C₁₋₆alkylene-CN, C₁₋₆ alkylene-NR₂, C₁₋₆alkylene-OR; halogen; —CN; —NO₂; —OR;
—SR; —NR₂; —S(O)₂R; —S(O)₂NR₂; —S(O)R; —S(O)NR₂; —C(O)R; —C(O)OR;
—C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R⁶ is independently selected from hydrogen, halogen, C₁₋₄ alkyl, and C₁₋₄ haloalkyl, or two R⁶ combine to form oxo;
each R⁷ is independently selected from hydrogen, halogen, and C₁₋₄ alkyl, or two R⁷ combine to form oxo;
each R⁹ is independently selected from hydrogen and C₁₋₄ alkyl;
each R is independently hydrogen, C₁₋₃ alkyl, C₂₋₆ alkenyl, C₂₋₄ alkynyl, C₁₋₃ alkoxy, C₁₋₃ haloalkyl, C₁₋₃ hydroxyalkyl, —C(O)N(CH₃)₂, —C(O)₂CH₃, —C(O)₂C(CH₃)₃, —C(O)₂CH(CH₃)₂, —S(O)₂CH₃, and C₀₋₆ alkylene-cyclic group, wherein the cyclic group is selected from:
phenyl;
a 3-7 membered saturated or partially unsaturated carbocyclic ring;
a 3-7 membered saturated or partially unsaturated heterocyclic ring having 1-2 heteroatoms independently selected from nitrogen, oxygen, and sulfur;
a 5-6 membered heteroaryl ring having 1-4 heteroatoms independently selected from nitrogen, oxygen, and sulfur;
an 8-10 membered bicyclic heteroaryl ring having 1-4 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-8 membered saturated or partially unsaturated bridged bicyclic ring having 0-3 heteroatoms independently selected from nitrogen, oxygen, and sulfur;
a 6-10 membered saturated or partially unsaturated spirocyclic ring having 0-3 heteroatoms independently selected from nitrogen, oxygen, and sulfur;
and 6-11 membered saturated or partially unsaturated bicyclic ring having 0-2 heteroatoms independently selected from nitrogen, oxygen, and sulfur;
wherein the cyclic group is substituted with 0, 1, 2, or 3 R^{S};
or
two R on the same atom together with the atom on which they are attached form a cyclic group selected from:
a 4-7 membered saturated or partially unsaturated carbocycyl;
a 4-7 membered saturated or partially unsaturated heterocyclyl having 1-3 heteroatoms independently selected from nitrogen, oxygen, and sulfur; and
a 5-6 membered heteroaryl ring having 1-3 heteroatoms independently selected from nitrogen, oxygen, and sulfur,
and the cyclic group is substituted with 0, 1, 2, or 3 R^{S}.

2. The compound of claim 1, having a structure of Formula 1-b-1, 1-c-1 or 1-d-1:

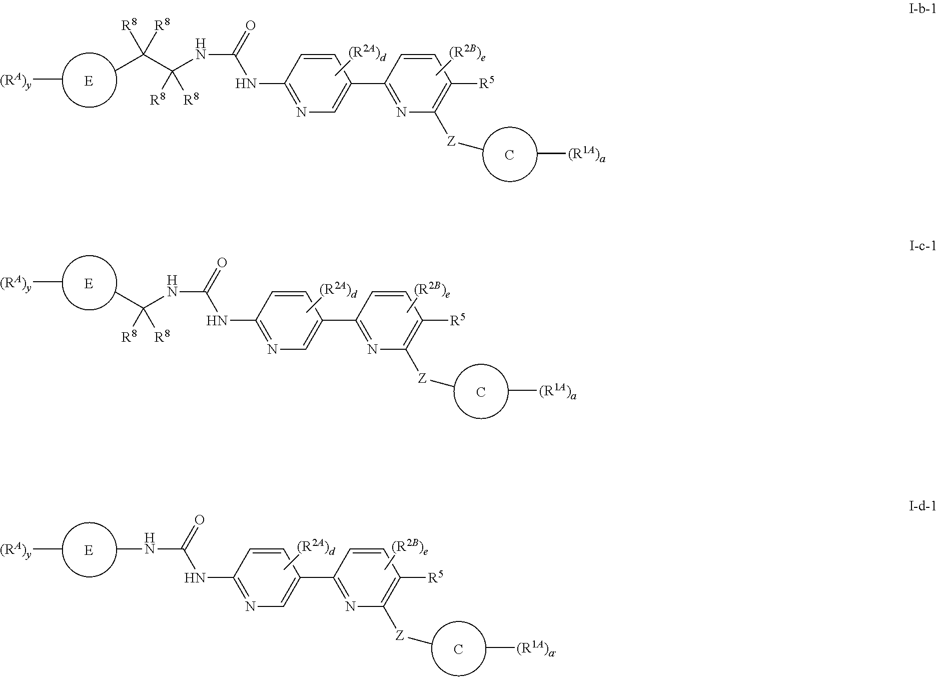

3. The compound of claim 1, having a structure of formula I-a-1: 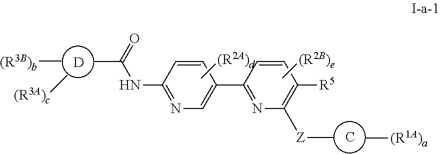
or a pharmaceutically acceptable salt thereof, wherein:
Ring C is a 5-membered heteroaryl having 1-3 heteroatoms independently selected from nitrogen, oxygen, and sulfur.

4. The compound of claim 1, wherein Ring D is

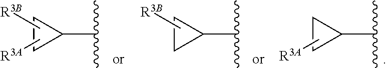

5. The compound of claim 1, having a structure of formula I-a-2: 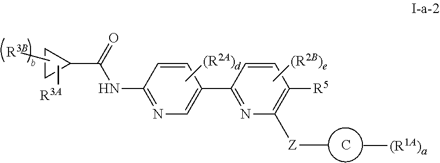
or a pharmaceutically acceptable salt thereof, wherein:
Ring C is a 5-membered heteroaryl having 1-3 heteroatoms independently selected from nitrogen, oxygen, and sulfur;
R^{3A} is a 5-6 membered heteroaromatic ring having 1-4 heteroatoms independently selected from nitrogen, oxygen, and sulfur; and
R^{2A} and R^{2B} are independently selected from F, CH₃ and Cl.

6. The compound of claim 1, wherein d is 0 or e is 0, or each of d and e is 0.

7-8. (canceled)

9. The compound of claim 1, wherein b is 0.

10. The compound of claim 1, wherein R^{3A} is

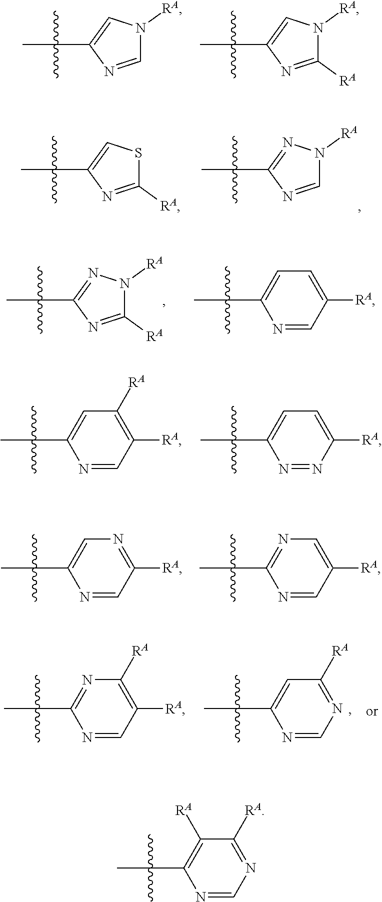

11. The compound of claim 10, wherein R^{3A} is

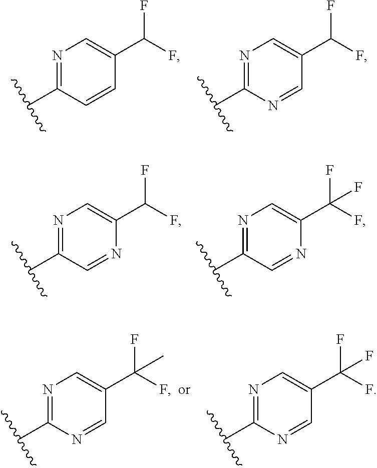

12. (canceled)

13. The compound of claim 1, wherein y is 1.

14. The compound of claim 1, wherein Ring C is a 5-membered heteroaryl having 1-3 heteroatoms independently selected from nitrogen, oxygen, and sulfur.

15. The compound of claim 1, having a structure of formula I-a-2: 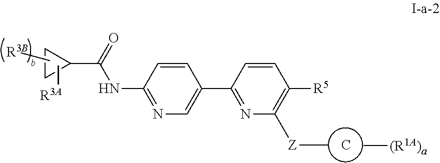
or a pharmaceutically acceptable salt thereof, wherein:
Ring C is a 5-membered heteroaryl having 1-3 heteroatoms independently selected from nitrogen, oxygen, and sulfur;
R^{3A} is a 5-6 membered heteroaromatic ring having 1-4 heteroatoms independently selected from nitrogen, oxygen, and sulfur; and
R^{S} is —CN or C₁₋₄ hydroxyalkyl.

16. The compound of claim 1, wherein Ring C is

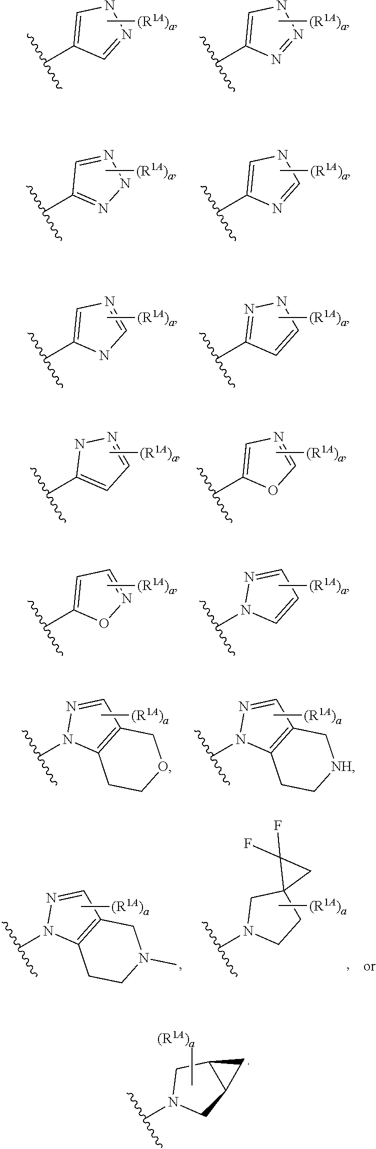

17-18. (canceled)

19. The compound of claim 1, wherein at least one R^{1A} is methyl,

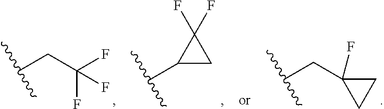

20. The compound of claim 1, wherein Z is a bond, —CH₂—CH₂—, —CH₂—, —O—, #C(O)—CH₂—, #O—CH₂—, #O—CH₂—CH₂—, #CH₂—O—, #CH₂—CH₂—O—, #NH—CH₂—, #NH—CH₂—CH₂—, #N(CH₃)—CH₂—, or #N(CH₃)—CH₂—CH₂—, wherein # is a direct bond to the pyridine ring.

21. The compound of claim 1, wherein R⁵ is C₁₋₄ hydroxyalkyl.

22. (canceled)

23. The compound of claim 1, having a structure as depicted in Table 2, or a pharmaceutically acceptable salt thereof.

24. (canceled)

25. A pharmaceutical composition comprising a compound of claim 1, or a pharmaceutically acceptable salt thereof, and a pharmaceutically acceptable carrier, adjuvant, or vehicle.

26-27. (canceled)

28. A method of treating an SIK2-mediated disease in a patient, comprising administering to said patient a therapeutically effective amount of the compound of claim 1.

29-32. (canceled)
